(19) United States
(12) Patent Application Publication
(10) Pub. No.: US 2006/0116356 A1
(43) Pub. Date: Jun. 1, 2006

(54) Phosphonate analogs of HIV integrase inhibitor compounds

(76) Inventors: Zhenhong R. Cai, Foster City, CA (US); Xiaowu Chen, San Mateo, CA (US); Maria Fardis, San Carlos, CA (US); Salman Y. Jabri, San Francisco, CA (US); Haolun Jin, Foster City, CA (US); Choung U. Kim, San Carlos, CA (US); Samuel E. Metobo, Newark, CA (US); Michael R. Mish, Foster City, CA (US); Richard M. Pastor, San Francisco, CA (US)

Correspondence Address: Schwegman, Lundberg, Woessner & Kluth, P.A., P.O. Box 2938, Minneapolis, MN, 55402, US

(21) Appl. No.: 11/106,363

(22) Filed: Apr. 14, 2005

Related U.S. Application Data
(60) Provisional application No. 60/562,678, filed on Apr. 14, 2004.

(51) Int. Cl. A61K 31/675; C07F 9/6544; C07F 9/6533

(52) U.S. Cl. 514/79; 514/90; 544/57; 544/157; 546/22

(57) ABSTRACT

Novel HIV integrase inhibitor compounds having at least one phosphonate group, protected intermediates thereof, and methods for inhibition of HIV-integrase are disclosed.

### FIELD OF THE INVENTION

The invention relates generally to phosphonate compounds with antiviral activity and more specifically with anti-HIV integrase properties.

### BACKGROUND OF THE INVENTION

AIDS is a major public health problem worldwide. Despite the unprecedented successes in the therapy of HIV infection, AIDS remains a major world health problem being the first cause of death in Africa and the fourth leading cause of death worldwide. Rapid emergence of drug-resistant HIV variants and severe side effects limit the efficacy of existing therapies. Although drugs targeting HIV viruses are in wide use and have shown effectiveness, toxicity and development of resistant strains have limited their usefulness. Assay methods capable of determining the presence, absence or amounts of HIV viruses are of practical utility in the search for inhibitors as well as for diagnosing the presence of HIV.

Human immunodeficiency virus (HIV) infection and related disease is a major public health problem worldwide. The retrovirus human immunodeficiency virus type 1 (HIV-1), a member of the primate lentivirus family (De Clercq E (1994) Annals of the New York Academy of Sciences, 724:438-456; Barre-Sinoussi F (1996) Lancet, 348:31-35), is generally accepted to be the causative agent of acquired immunodeficiency syndrome (AIDS) Tarrago et al FASEB Journal 1994, 8:497-503). AIDS is the result of repeated replication of HIV-1 and a decrease in immune capacity, most prominently a fall in the number of CD4+ lymphocytes. The mature virus has a single stranded RNA genome that encodes 15 proteins (Frankel et al (1998) Annual Review of Biochemistry, 67:1-25; Katz et al (1994) Annual Review of Biochemistry, 63:133-173), including three key enzymes: (i) protease (Prt) (von der Helm K (1996) Biological Chemistry, 377:765-774); (ii) reverse transcriptase (RT) (Hottiger et al (1996) Biological Chemistry Hoppe-Seyler, 377:97-120), an enzyme unique to retroviruses; and (iii) integrase (Asante et al (1999) Advances in Virus Research 52:351-369; Wlodawer A (1999) Advances in Virus Research 52:335-350; Esposito et al (1999) Advances in Virus Research 52:319-333). Protease is responsible for processing the viral precursor polyproteins, RT is the key enzyme in the replication of the viral genome, and integrase, a viral encoded protein, is responsible for the integration of the double stranded DNA form of the viral genome into host DNA.

Until 1995, the only drugs approved in the United States were nucleoside inhibitors of RT (Smith et al (1994) Clinical Investigator, 17:226-243). Since then, two new classes of agents, protease inhibitors (PI) and non-nucleoside RT inhibitors (NNRTI), and more than a dozen new drugs have been approved (Johnson et al (2000) Advances in Internal Medicine, 45 (1-40; Porche D J (1999) Nursing Clinics of North America, 34:95-112). There are now three classes of drugs available: (1) the original nucleoside RT inhibitors, (2) protease inhibitors (PI), and (3) the non-nucleoside RT inhibitors (NNRTI).

An essential step in HIV infection is the integration of the viral genome into the host cell chromosomes within the nucleus. Unlike other retroviruses, HIV can transport its genetic material, in the form of the large nucleoprotein pre-integration complex (PIC), into the nucleus through the intact nuclear envelope and infect non-dividing cells such as macrophages and microglial cells. Although several different components of the PIC have been implicated in its nuclear import, the mechanism of nuclear entry remains unclear (Piller et al (2003) Current Drug Targets 4:409-429; Debyser et al (2002) Antiviral Chemistry & Chemotherapy 13:1-15). Specifically inhibiting PIC nuclear import would likely block HIV infection in non-dividing cells, this important step of HIV replication is of great interest as a drug target. The identification of compounds unambiguously affecting HIV replication by targeting integrase supports the potential of this crucial viral enzyme as a drug target. Certain HIV integrase inhibitors have been disclosed which block integration in extracellular assays and exhibit antiviral effects against HIV-infected cells (Anthony, et al WO 02/30426; Anthony, et al WO 02/30930; Anthony, et al WO 02/30931; WO 02/055079 A2 A3; Zhuang, et al WO 02/36734; U.S. Pat. No. 6,395,743; U.S. Pat. No. 6,245,806; U.S. Pat. No. 6,271,402; Fujishita, et al WO 00/039086; Uenaka et al WO 00/075122; Selnick, et al WO 99/62513; Young, et al WO 99/62520; Payne, et al WO 01/00578; Parrill, A. L. (2003) Current Medicinal Chemistry 10:1811-1824; Gupta et al (2003) Current Medicinal Chemistry 10:1779-1794; Maurin et al (2003) Current Medicinal Chemistry 10:1795-1810; Jing, et al Biochemistry (2002) 41:5397-5403; Pais, et al Jour. Med. Chem. (2002) 45:3184-94; Goldgur, et al Proc. Natl. Acad. Sci. U.S.A. (1999) 96:13040-13043; Espeseth, et al Proc. Natl. Acad. Sci. U.S.A. (2000) 97:11244-11249). For reviews, see: Neamati (2002) Expert Opin. Ther. Patents 12(5):709-724; Pommier et al (1999) Advances in Virus Research 52:427-458; Young (2001) Current Opinion in Drug Discovery & Development 4(4):402-410; Neamati et al (2001) “Human Immunodeficiency Virus type 1 Integrase Targeted Inhibitor Design”, Antiretroviral Therapy, E. De Clercq, Ed., ASM Press, Washington, D.C.; Pani et al (2000) Current Pharm. Design 6:569-584; Pommier et al (2000) Antiviral Res. 47(3):139-148; De Clercq E. (2002) Medicinal Research Reviews 22(6):531-565.

HIV integrase inhibitory compounds with improved antiviral and pharmacokinetic properties are desirable, including enhanced activity against development of HIV resistance, improved oral bioavailability, greater potency and extended effective half-life in vivo (Nair, V. “HIV integrase as a target for antiviral chemotherapy” Reviews in Medical Virology (2002) 12(3):179-193). Three-dimensional quantitative structure-activity relationship studies and docking simulations (Buolainwini, et al Jour. Med. Chem. (2002) 45:841-852) of conformationally-restrained cinnamoyl-type integrase inhibitors (Artico, et al Jour. Med. Chem. (1998) 41:3948-3960) have correlated hydrogen-bonding interactions to the inhibitory activity differences among the compounds.

Phase II clinical studies candidate, S-1360 (Shionogi-GlaxoSmithKline Pharmaceuticals LLC) is the furthest advanced HIV integrase inhbitor to date. Animal toxicity studies have been reported for other candidates, L-731,988 and L-708,906, by Merck.

There is a need for anti-HIV therapeutic agents, i.e. drugs having improved antiviral and pharmacokinetic properties with enhanced activity against development of HIV resistance, improved oral bioavailability, greater potency and extended effective half-life in vivo. New HIV inhibitors should be active against mutant HIV strains, have distinct resistance profiles, fewer side effects, less complicated dosing schedules, and orally active. In particular, there is a need for a less onerous dosage regimen, such as one pill, once per day. Although drugs targeting HIV protease are in wide use and have shown effectiveness, particularly when employed in combination, toxicity and development of resistant strains have limited their usefulness (Palella, et al N. Engl. J. Med. (1998) 338:853-860; Richman, D. D. Nature (2001) 410:995-1001).

Combination therapy with HIV inhibitors has proven to be highly effective in suppressing viral replication to unquantifiable levels for a sustained period of time. Also, combination therapy with RT and protease inhibitors have shown synergistic effects in suppressing HIV replication. Unfortunately, many patients currently fail combination therapy due to the development of drug resistance, non-compliance with complicated dosing regimens, pharmacokinetic interactions, toxicity, and lack of potency. Therefore, there is a need for HIV integrase inhibitors that are synergistic in combination with other HIV inhibitors, or show chemical stability in combination formulations.

Improving the delivery of drugs and other agents to target cells and tissues has been the focus of considerable research for many years. Though many attempts have been made to develop effective methods for importing biologically active molecules into cells, both in vivo and in vitro, none has proved to be entirely satisfactory. Optimizing the association of the inhibitory drug with its intracellular target, while minimizing intercellular redistribution of the drug, e.g. to neighboring cells, is often difficult or inefficient.

Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., cytotoxic agents and other anti-cancer or anti-viral drugs) that can be administered. By comparison, although oral administration of drugs is generally recognized as a convenient and economical method of administration, oral administration can result in either (a) uptake of the drug through the cellular and tissue barriers, e.g. blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment includes avoiding the general physiological effects of inappropriate delivery of such agents to other cells and tissues, such as uninfected cells. Intracellular targeting may be achieved by methods and compositions, including prodrugs (Krise et al (1996) Advanced Drug Delivery Reviews 19:287-310), which allow accumulation or retention of biologically active agents inside cells.

### SUMMARY OF THE INVENTION

The present invention provides novel compounds with HIV integrase activity, i.e. novel human retroviral integrase inhibitors. Therefore, the compounds of the invention may inhibit retroviral integrases and thus inhibit the replication of the virus. They are useful for treating human patients infected with a human retrovirus, such as human immunodeficiency virus (strains of HIV-1 or HIV-2) or human T-cell leukemia viruses (HTLV-I or HTLV-II) which results in acquired immunodeficiency syndrome (AIDS) and/or related diseases. The present invention includes novel phosphonate HIV integrase inhibitor compounds and phosphonate analogs of known experimental integrase inhibitors. The compounds of the invention optionally provide cellular accumulation as set forth below.

The present invention relates generally to the accumulation or retention of therapeutic compounds inside cells. The invention is more particularly related to attaining high concentrations of phosphonate-containing molecules in HIV infected cells. Intracellular targeting may be achieved by methods and compositions which allow accumulation or retention of biologically active agents inside cells. Such effective targeting may be applicable to a variety of therapeutic formulations and procedures.

Compositions of the invention include new HIV integrase inhibitor compounds having at least one phosphonate group. The compositions of the invention thus include all known approved, experimental, and proposed HIV integrase inhibitors, that do not already comprise a phosphonate group, with at least one phosphonate group covalently attached. Experimental HIV integrase inhibitors include those reviewed in: Dayam et al (2003) Current Pharmaceutical Design 9:1789-1802; De Clercq E. (2002) Biochimica et Biophysica Acta 1587(2-3):258-275; Nair, V. (2002) Reviews in Medical Virology 12(3):179-193; Neamati, N. (2002) Expert Opinion on Therapeutic Patents 12(5):709-724; Asante-Appiah et al (1997) Antiviral Res. 36:139-156; Dubrovsky et al (1995) Mol. Med. 1:217-230; Popov et al (1998) EMBO J. 17:909-917; Farnet et al (1996) AIDS 10(Suppl. A):S3-S11; Hansen et al (1998) Genet. Eng. 20:41-61; Bukrinsky, M. (1997) Drugs Future 22:875-883; Neamati et al (2000) Adv. Pharmacol. 49:147-163; Pommier et al (1999) Adv. Virus Res. 52:427-458; Neamati et al (1997) Drug Discovery Today 2:487-498; Nicklaus et al (1997) J. Med. Chem. 40:920-929; Pommier et al (1997) Antivir. Chem. Chemother. 8:483-503; Robinson J. (1998) Infect. Med. 15:129-137; Thomas et al (1997) Trends Biotechnol. 15:167-172.

The invention includes novel phosphonate analogs of the following experimental HIV integrase inhibitors in Groups I to XXXIX.
I. In one aspect, the invention includes tricyclic phosphonate compounds represented by the following structure, Formula I: 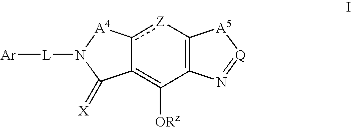
II. In one aspect, the invention includes phosphonate analogs of aza-quinolinol compounds represented by the Formula II: 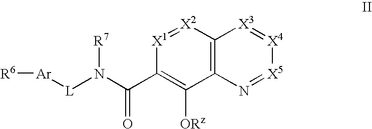
III. In one aspect, the invention includes phosphonate analogs of quinoline compounds represented by the Formula III: 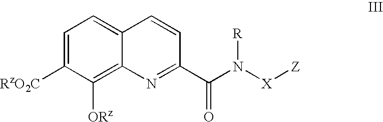
IV. In one aspect, the invention includes phosphonate analogs of 4,5-dihydroxypyrimidine, 6-carboxamide compounds having Formula IV: 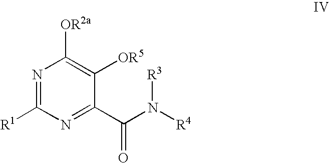
V. In one aspect, the invention includes phosphonate analogs of 3-N-substituted, 5-hydroxypyrimidinone, 6-carboxamide compounds having Formula V: 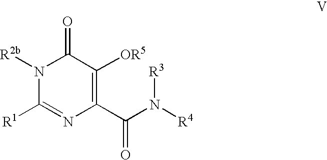
VI. In one aspect, the invention includes phosphonate analogs of 1,3 diketo compounds having Formula VI: 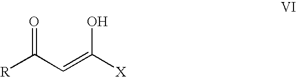
VII. In one aspect, the invention includes phosphonate analogs of 2,5 diarylsubstituted, furan compounds having Formula VII: 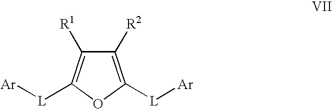
VIII. In one aspect, the invention includes phosphonate analogs of 2,5 substituted, diketo-furan compounds having Formula VIII: 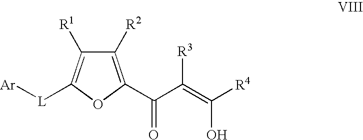
IX. In one aspect, the invention includes phosphonate analogs of catechol compounds including caffeic acid phenylethyl ester (CAPE) compounds having Formula IX: 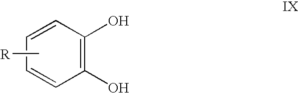
X. Catechol compounds IX include phosphonate analogs of styryl catechol compounds and analogs of chicoric acid. Phosphonate analogs of styryl catechol compounds generally have Formula X: 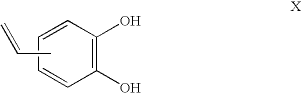
XI. In one aspect, the invention includes phosphonate analogs of benzimidazole compounds and bis-benzimidazole compounds having Formula XI: 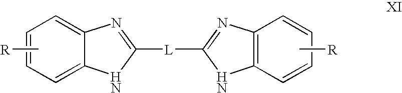
XII. In one aspect, the invention includes phosphonate analogs of indoloquinoxaline compounds having Formula XII: 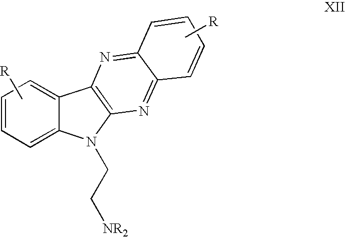
XIII. In one aspect, the invention includes phosphonate analogs of acridine compounds including phosphonate analogs of bis-acridine compounds having Formula XIII: 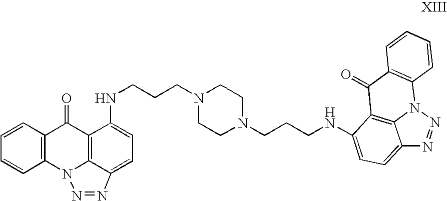
XIV. In one aspect, the invention includes phosphonate analogs of polyamide, DNA binding compounds, such as polypyrrole amide phosphonate oligomers having Formula XIV: 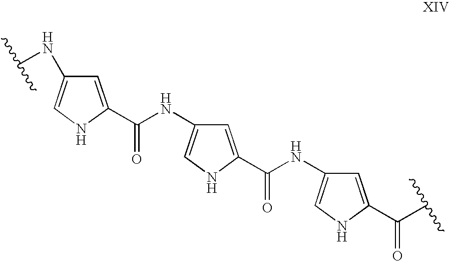
   where the wavy lines 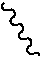
   indicate the depicted structure is a substructure of a repeating polymer molecule.
XV. In one aspect, the invention includes phosphonate analogs of [6,6] bicyclic compounds, including integramycins and fungal metabolites having Formula XV: 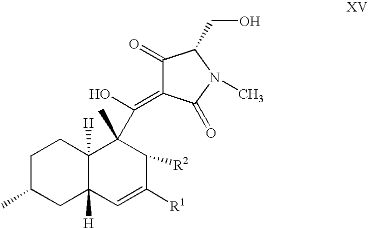
XVI. In one aspect, the invention includes phosphonate analogs of [6,6] bicyclic terpenoid compounds having Formula XVI: 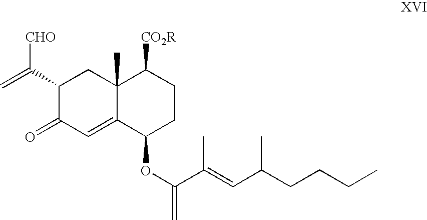
XVII. In one aspect, the invention includes phosphonate analogs of aurintricarboxylic acid compounds having Formula XVII: 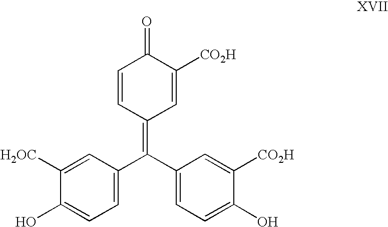
XVIII. In one aspect, the invention includes phosphonate analogs of integrastatin compounds having Formula XVIII: 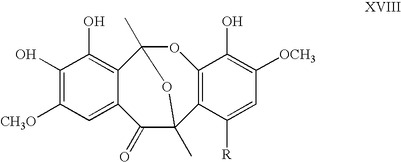
XIX. In one aspect, the invention includes phosphonate analogs of 6-(arylazo)pyridoxal-5-phosphate compounds having Formula XIX: 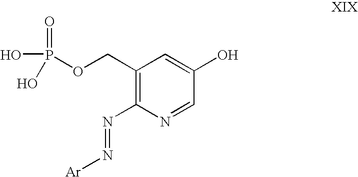
XX. In one aspect, the invention includes phosphonate analogs of 1,3-oxazine-, 1,3-thiazine-, pyran-, 1,4-oxazepine-, and 1,4-thiazepine-fused naphthalene compounds having Formula XX: 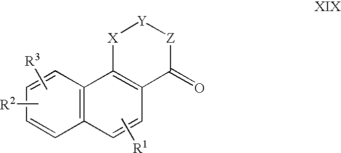
XXI. In one aspect, the invention includes phosphonate analogs of chaetochromin compounds derived from chaetochromin fermentation products and their chemically modified derivatives including naphtho-γ-pyrones having Formula XXI: 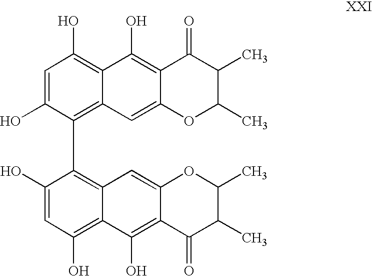
XXII. In one aspect, the invention includes phosphonate analogs of hydroxyphenylundecane compounds derived from fermentation products and their chemically modified derivatives including integracins having Formula XXII: 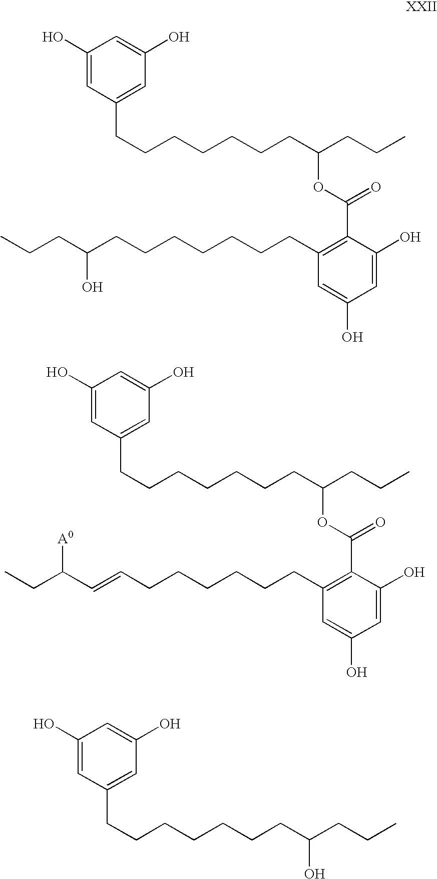
XXIII. In one aspect, the invention includes phosphonate analogs of: (i) tetracyclic steroidal compounds derived from fermentation products and their chemically modified derivatives; and (ii) tetracyclic triterpenoid compounds having Formula XXIII: 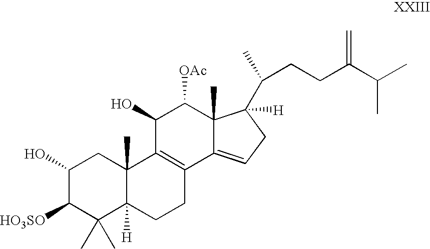
XXIV. In one aspect, the invention includes phosphonate analogs of plant natural products including: (i) glycerrhenitic and betulonic acids; (ii) compounds from Coleus parvifolius Benth.; (iii) eudesmane-type sesquiterpenes and aporphine alkaloid lindechunines from Lindera chunii roots including hemandonine, laurolistine, 7-oxohemangerine and lindechunine A; and (iv) lithospermic acid.
XXV. In one aspect, the invention includes phosphonate analogs of tetracyclic aromatic ketone compounds and derived from fungal cultures and fungus, and their chemically modified derivatives.
XXVI. In one aspect, the invention includes phosphonate analogs of aromatic compounds derived from lichen extracts, and their chemically modified derivatives.
XXVII. In one aspect, the invention includes phosphonate analogs of salicylhydrazide and mercaptosalicylhydrazide compounds.
XXVIII. In one aspect, the invention includes phosphonate analogs of thiazolothiazepine compounds.
XXIX. In one aspect, the invention includes phosphonate analogs of benzodiazepine hydrazide compounds.
XXX. In one aspect, the invention includes phosphonate analogs of coumarin compounds, including Lamellarin-type marine natural products.
XXXI. In one aspect, the invention includes phosphonate analogs of brominated polyacetylene marine natural products from sponges such as Diplastrella sp.
XXXII. In one aspect, the invention includes phosphonate analogs of cobalamin compounds.
XXXIII. In one aspect, the invention includes phosphonate analogs of hydroxylated aromatic compounds, including: tetracycline compounds; anthraquinones and naphthoquinones; and flavones, flavanones, flavanols, and flavanoids including thalassiolins and benzopyrano-oxopyrimidotetrahydrothiazines.
XXXIV. In one aspect, the invention includes phosphonate analogs of various sulfur-containing compounds including phosphonate analogs of: polyanionic sulfonate suramin and dextran sulfate; diaryl sulfones; sulfonamides; aromatic disulfides; and 2-mercaptobenzenesulfonamides.
XXXV. In one aspect, the invention includes phosphonate analogs of symmetrical pentamidine compounds derived from serine protease inhibitors.
XXXVI. In one aspect, the invention includes phosphonate analogs of nucleic acid compounds. Nucleic acid phosphonate compounds include: (a) nucleosides and nucleotides; dinucleotides, including 5H-pyrano[2,3-d:-6,5-d′]dipyrimidines; (b) oligonucleotides; and (c) analogs thereof, with one or more phosphonate groups. Nucleic acid analogs include nucleobase, sugar, and internucleotide phosphate analogs.
XXXVII. In one aspect, the invention includes phosphonate analogs of amino acids and peptides.
XXXVIII. In one aspect, the invention includes phosphonate analogs of polyketide natural products including Xanthoviridicatins isolated from a fermentation broth of an endophytic strain of Penicillium chrysogenum.
XXXIX. In one aspect, the invention includes phosphonate analogs of polyketide natural products including cytosporic acid, australifungin and australifunginol isolated from a fermentation broth of the filamentous fungus.

The compounds of the invention, including Formulas I-XXXIX, are substituted with one or more covalently attached phosphonate groups. Formulas I-XXXIX are “scaffolds”, i.e. substructures which are common to the specific compounds encompassed therein.

It is to be understood that the scope of the invention includes compounds in which hydrogen atoms at any of the various positions in Formulas I-XXXIX are independently substituted with non-hydrogen substituents. In particular, the variable positions on the scaffolds of Formulas I-XXXIX and experimental HIV integrase inhibitors of Groups I-XXXIX are independently substituted with the non-hydrogen substituents described herein.

The invention includes pharmaceutically acceptable salts of Formulas I-XXXIX, and all enol and tautomeric resonance isomers thereof. Except where the stereochemistry is explicit, the compounds of the invention include all stereoisomers; i.e. each enantiomer, diastereomer, and atropisomer in purified form, or racemic and isomerically enriched mixtures.

The invention provides a pharmaceutical composition comprising an effective amount of a compound selected from Formulas I-XXXIX, or a pharmaceutically acceptable salt thereof, in a formulation, i.e. in combination with a pharmaceutically acceptable excipient, diluent or carrier.

The invention includes combination formulations including the compounds of the invention, with other active ingredients that treat or prevent HIV infections. Such combination formulations may be a fixed dose of two or more active ingredients, including at least one compound of the invention.

This invention also pertains to a method of increasing cellular accumulation and retention of drug compounds, thus improving their therapeutic and diagnostic value.

The use of the compounds of the invention in an HIV infected patient, or in a sample suspected of containing HIV, anticipates all metabolites of the compounds so administered which occur by solvolysis, hydrolysis, photolysis, or by enzymatic action which converts or degrades the administered compound into, e.g. an activated form, an incorporated form, a cleaved form, or a metabolite for excretion.

The invention also provides a method of inhibiting HIV, comprising administering to a mammal infected with HIV (HIV positive) an amount of a compound of Formulas I-XXXIX, effective to inhibit the growth of said HIV infected cells.

The invention also provides a compound selected from Formulas I-XXXIX for use in medical therapy, as well as the use of a compound of Formulas I-XXXIX for the manufacture of a medicament useful for: (1) the treatment of AIDS or ARC (AIDs related complex); or (2) the prophylaxis of infection by HIV.

The invention also provides processes and novel intermediates disclosed herein which are useful for preparing compounds of the invention. Other aspects of the invention are novel methods for synthesis, i.e. preparation, of the compounds of the invention.

Some of the compounds of Formulas I-XXXIX are useful to prepare other compounds of Formulas I-XXXIX.

One aspect of the invention is the inhibition of the activity of HIV integrase by a method comprising the step of treating a sample suspected of containing HIV virus with a compound or composition of the invention.

Other aspects of the invention are formulation compositions of the compounds of the invention, as well as methods of formulating the compositions.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims.

Definitions

Unless stated otherwise, the following terms and phrases as used herein are intended to have the following meanings:

When tradenames are used herein, applicants intend to independently include the tradename product and the active pharmaceutical ingredient(s) of the tradename product.

The terms “phosphonate” and “phosphonate group” mean a functional group or moiety within a molecule that comprises at least one phosphorus-carbon bond, and at least one phosphorus-oxygen double bond. The phosphorus atom is further substituted with oxygen, sulfur, or nitrogen substituents. These substituents may be part of a prodrug moiety. As defined herein, “phosphonate” and “phosphonate group” include moieties with phosphonic acid, phosphonic monoester, phosphonic diester, phosphonamidate, and phosphonthioate functional groups.

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound. For a review of phosphorus prodrugs, see: Krise et al (1996) Advanced Drug Delivery Reviews 19:287-310.

“Pharmaceutically acceptable prodrug” refers to a compound that is metabolized in the host, for example hydrolyzed or oxidized, by either enzymatic action or by general acid or base solvolysis, to form an active ingredient. Typical examples of prodrugs of the compounds of the invention have biologically labile protecting groups on a functional moiety of the compound. Prodrugs include compounds that can be oxidized, reduced, aminated, deaminated, esterified, deesterified, alkylated, dealkylated, acylated, deacylated, phosphorylated, dephosphorylated, photolyzed, hydrolyzed, or other functional group change or conversion involving forming or breaking chemical bonds on the prodrug.

“Prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy.

Exemplary prodrug moieties include the hydrolytically sensitive or labile acyloxymethyl esters —CH₂C(═O)R⁹ and acyloxymethyl carbonates —CH₂C(═O)OR⁹ where R⁹ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al (1983) J. Pharm. Sci. 72: 324; also U.S. Pat. Nos. 4,816,570, 4,968,788, 5,663,159 and 5,792,756. In certain compounds of the invention, a prodrug moiety is part of a phosphonate group. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the combinations of the invention. An exemplary acyloxymethyl ester is pivaloyloxymethoxy, (POM) —CH₂C(═O)C(CH₃)₃. Exemplary acyloxymethyl carbonate prodrug moieties are pivaloyloxymethylcarbonate (POC) —CH₂C(═O)OC(CH₃)₃ and (pivoxil) —CH₂C(═O)OCH(CH₃)₂.

The phosphonate group may be a phosphonate prodrug moiety. The prodrug moiety may be sensitive to hydrolysis, such as, but not limited to a pivaloyloxymethyl carbonate (POC) or POM group. Alternatively, the prodrug moiety may be sensitive to enzymatic potentiated cleavage, such as a lactate ester or a phosphonamidate-ester group.

Aryl esters of phosphorus groups, especially phenyl esters, are reported to enhance oral bioavailability (DeLambert et al (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphonic acid. In some cases, substituents at the ortho- or para-position may accelerate the hydrolysis. Benzyl analogs with an acylated phenol or an alkylated phenol may generate the phenolic compound through the action of enzymes, e.g. esterases, oxidases, etc., which in turn undergoes cleavage at the benzylic C—O bond to generate the phosphoric acid and the quinone methide intermediate. Examples of this class of prodrugs are described by Mitchell et al (1992) J. Chem. Soc. Perkin Trans. 12345; Brook et al WO 91/19721. Still other benzylic prodrugs have been described containing a carboxylic ester-containing group attached to the benzylic methylene (Glazier et al WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al (1993) Antiviral Res., 22:155-174; Benzaria et al (1996) J. Med. Chem. 39:4958). Cyclic phosphonate esters have also been described as prodrugs of phosphorus-containing compounds (Erion et al, U.S. Pat. No. 6,312,662).

“Protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous.

Any reference to any of the compounds of the invention also includes a reference to a physiologically acceptable salt thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of an hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂)

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH),

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently —H, alkyl, aryl, heterocycle, protecting group or prodrug moiety. Alkylene, alkenylene, and alkynylene groups may also be similarly substituted.

“Heteroaryl” and “Heterocycle” refer to a ring system in which one or more ring atoms is a heteroatom, e.g. nitrogen, oxygen, and sulfur. The heterocycle radical comprises 5 to 14 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

One embodiment of the bis-tetrahydrofuranyl group is:

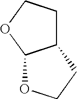

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

“Nucleobase” means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases: adenine, guanine, cytosine, uracil, thymine, and analogs of the naturally occurring nucleobases, e.g. 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines (U.S. Pat. Nos. 6,143,877 and 6,127,121; WO 01/38584), and ethenoadenine (Fasman (1989) in Practical Handbook of Biochemistry and Molecular Biology, pp. 385-394, CRC Press, Boca Raton, Fla.). Nucleobases include the five-membered heterocyclic nucleobase analogs disclosed in WO 03/073989 A2 such as substituted triazoles:

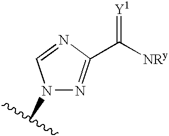

Nucleobases also include any of the above nitrogen-containing heterocyclic moieties which have one or more protecting groups (PG) covalently attached to reactive functionality, such as the N-2 or N-6 exocyclic amino of purines, the N-3 or N-4 nitrogen of pyrimidines, or the 0-6 oxygen of guanine type nucleobases. Suitable nucleobase protecting groups include amide-forming groups such as benzoyl or isobutyramide, acetamidine-forming groups, and formamidine-forming groups such as dimethylformamidyl (dmf). Reactive functionality of nucleobases can also be protected with transient groups such as 6-chloro of purines.

Nucleobases are typically attached in the configurations of naturally-occurring nucleic acids to the sugar moiety through a covalent bond between the 1′ carbon of the sugar moiety and the N-9 of purines, e.g. adenin-9-yl and guanin-9-yl, or N−1 of pyrimidines, e.g. thymin-1-yl and cytosin-1-yl (Blackburn, G. and Gait, M. Eds. “DNA and RNA structure” in Nucleic Acids in Chemistry and Biology, 2^{nd} Edition, (1996) Oxford University Press, pp. 15-81).

“Linker” or “link” means a chemical moiety comprising a covalent bond or a chain of atoms that covalently attaches a phosphonate group to a drug. In various embodiments, a linker is specified as L. Linkers include a divalent radical such as an alkyldiyl, an aryldiyl, or a heteroaryldiyl; or portions of substituent A¹ enumerated in Formulas I-XXXIX, which include moieties such as: —(CR₂)ₙO(CR₂)ₙ—, repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

HIV Integrase Inhibitor Phosphonate Compounds

The compounds of the invention include those with HIV integrase inhibitory activity. In particular, the compounds include HIV integrase inhibitors. The compounds of the inventions bear at least one phosphonate group, selected from: phosphonic acid, phosphonate monoester, phosphonate diester, phosphonamidate, phosphonthioate, phosphondithioate, phosphonamidate-ester prodrug, or a phosphonbisamidate-ester (Jiang et al, U.S. 2002/0173490 A1), any of which may be a prodrug moiety.

The compositions of the invention include all known approved, experimental, and proposed HIV integrase inhibitors, that do not already comprise a phosphonate group, with at least one phosphonate group covalently attached. The invention includes novel phosphonate analogs of the following experimental HIV integrase inhibitors in Groups I to XXXIX that do not already comprise a phosphonate group. Embodiments of the invention include phosphonate analogs of compounds that fall within the generic scope of the documents cited in Groups I to XXXIX.

It is to be understood that the scope of the invention includes compounds in which hydrogen atoms at any of the various positions in Formulas I-XXXIX are independently substituted with non-hydrogen substituents, including those designated with A⁰, A¹, A², and A³.

The invention includes pharmaceutically acceptable salts of Formulas I-XXXIX, and all enol and tautomeric resonance isomers thereof.

The compounds of the invention, including Formulas I-XXXIX, are substituted with one or more covalently attached groups, including at least one phosphonate group, i.e. A¹ or A³. Formulas I-XXXIX are “scaffolds”, i.e. substructures which are common to the specific compounds encompassed therein.

Formulas I-XXXIX are substituted with one or more covalently attached A⁰ groups, including simultaneous substitutions at any or all A⁰.

A⁰ is A¹, A² or W³.

Compounds of Formulas I-XXXIX include at least one A¹ and thus include at least one A³.

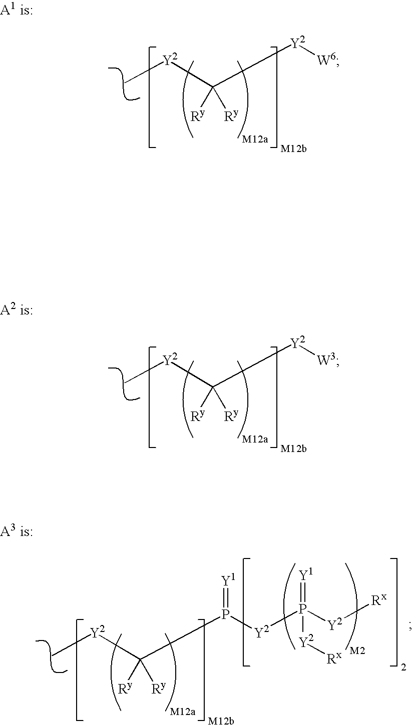

where:

Y¹ is independently O, S, NR^{x}, N(O)(R^{x}), N(OR^{x}), N(O)(OR^{x}), or N(N(R^{x})₂);

Y² is independently a bond, O, NR^{x}, N(O)(R^{x}), N(OR^{x}), N(O)(OR^{x}), N(N(R^{x})₂), —S(O)— (sulfoxide), —S(O)₂— (sulfone), —S— (sulfide), or —S—S— (disulfide);

M2 is 0, 1 or 2;

M12a is 1, 2, 3, 4, 5, 6, 7, 8, 9, 10, 11, or 12; and

M12b is 0, 1, 2, 3, 4, 5, 6, 7, 8, 9, 10, 11, or 12.

Further for the purposes of A¹, A² and A³:

R^{y} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or where taken together at a carbon atom, two vicinal R^{y} groups form a carbocycle or a heterocycle. Alternatively, taken together at a carbon atom, two vicinal R^{y} groups form a ring, i.e. a spiro carbon. The ring may be all carbon atoms, for example, cyclopropyl, cyclobutyl, cyclopentyl, or cyclohexyl, or alternatively, the ring may contain one or more heteroatoms, for example, piperazinyl, piperidinyl, pyranyl, or tetrahydrofuryl;

R^{x} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or the formula:

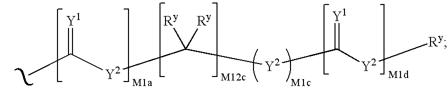

where M1a, M1c, and M1d are independently 0 or 1, and M12c is 0, 1, 2, 3, 4, 5, 6, 7, 8, 9, 10, 11 or 12;

W³ is W⁴ or W⁵;

W⁴ is R⁵, —C(Y¹)R⁵, —C(Y¹)W⁵, —SO₂R⁵, or —SO₂W⁵;

W⁵ is carbocycle or heterocycle wherein W⁵ is independently substituted with 0 to 3 R² groups;

W^{3a} is W^{4a} or W^{5a};

W^{4a} is R^{5a}, —C(Y¹)R^{5a}, —C(Y¹)W^{5a}, —SO₂R^{5a}, or —SO₂W^{5a};

W^{5a} is a multivalent substituted carbocycle or heterocycle wherein W^{5a} is independently substituted with 0 to 3 R² groups;

W⁶ is W^{3a} independently substituted with 1, 2, or 3 A3 groups;

R¹ is independently H or alkyl of 1 to 18 carbon atoms;

R² is independently H, R³ or R⁴ wherein each R⁴ is independently substituted with 0 to 3 R³ groups. Alternatively, taken together at a carbon atom, two R² groups form a ring, i.e. a spiro carbon. The ring may be, for example, cyclopropyl, cyclobutyl, cyclopentyl, or cyclohexyl. The ring may be substituted with 0 to 3 R³ groups;

R³ is R^{3a}, R^{3b}, R^{3c} or R^{3d}, provided that when R³ is bound to a heteroatom, then R³ is R^{3c} or R^{3d};

R^{3a} is F, Cl, Br, I, —CN, N₃ or —NO₂;

R^{3b} is Y;

R^{3c} is R^{x}, —N(R^{x})₂, —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)N(R^{x})₂, —SC(Y¹)R^{x}, —SC(Y¹)OR^{x}, —SC(Y¹)N(R^{x})₂, —N(R^{x})C(Y¹)R^{x}, —N(R^{x})C(Y¹)OR^{x}, or —N(R^{x})C(Y¹)N(R^{x})₂;

R^{3d} is —C(Y¹)R^{x}, —C(Y¹)OR^{x} or —C(Y¹)N(R^{x})₂;

R⁴ is an alkyl of 1 to 18 carbon atoms, alkenyl of 2 to 18 carbon atoms, or alkynyl of 2 to 18 carbon atoms;

R⁵ is R⁴ wherein each R⁴ is substituted with 0 to 3 R³ groups; and

R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups.

R is independently selected from H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, C₂-C₂₀ substituted heterocycle, phosphonate, phosphate, polyethyleneoxy, a protecting group, L-A³, and a prodrug moiety.

Substituted alkyl, substituted alkenyl, substituted alkynyl, substituted aryl, and substituted heterocycle are independently substituted with one or more substituents selected from F, Cl, Br, I, OH, amino (—NH₂), ammonium (—NH₃⁺), alkylamino (—NHR), dialkylamino (—NR₂), trialkylammonium (—NR₃⁺), C₁-C₈ alkyl, C₁-C₈ alkylhalide, carboxylate, thiol (—SH), sulfate (—OSO₃R), sulfamate, sulfonate (—SO₃R), 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—COOR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, and a prodrug moiety.

L is a bond or any linker which covalently attaches a phosphonate group to a drug scaffold. For example, L may be a bond, O, S, S—S (disulfide), S(═O) (sulfoxide), S(═O)₂ (sulfone), S(═O)₂NR (sulfonamide), NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, —(CR₂)ₙO(CR₂)ₙ—, C(═O)NH, OC(═O)NH, NHC(═O)NH, C(═O), C(═O)NH(CH₂)ₙ, or (CH₂CH₂O)ₙ, where n may be 1, 2, 3, 4, 5, or 6.

Further for the purposes of A¹, A² and A³ and other substituents described in the compounds of the invention:

Carbocycles and heterocycles may be independently substituted with 0 to 3 R² groups. Carbocycles and heterocycles may be a saturated, unsaturated or aromatic ring comprising a mono- or bicyclic carbocycle or heterocycle. Carbocycles and heterocycles may have 3 to 10 ring atoms, e.g., 3 to 7 ring atoms. The W⁵ rings are saturated when containing 3 ring atoms, saturated or mono-unsaturated when containing 4 ring atoms, saturated, or mono- or di-unsaturated when containing 5 ring atoms, and saturated, mono- or di-unsaturated, or aromatic when containing 6 ring atoms.

Carbocycles and heterocycles include, but are not limited to, examples such as:

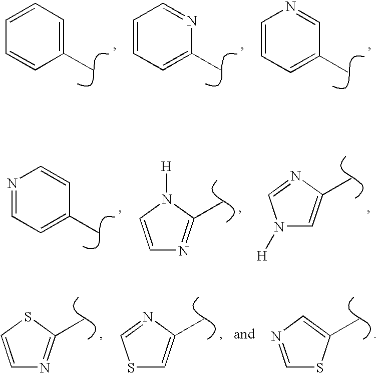

Carbocycles and heterocycles may be independently substituted with 0 to 3 groups, as defined above. For example, substituted carbocycles (Ar) include: 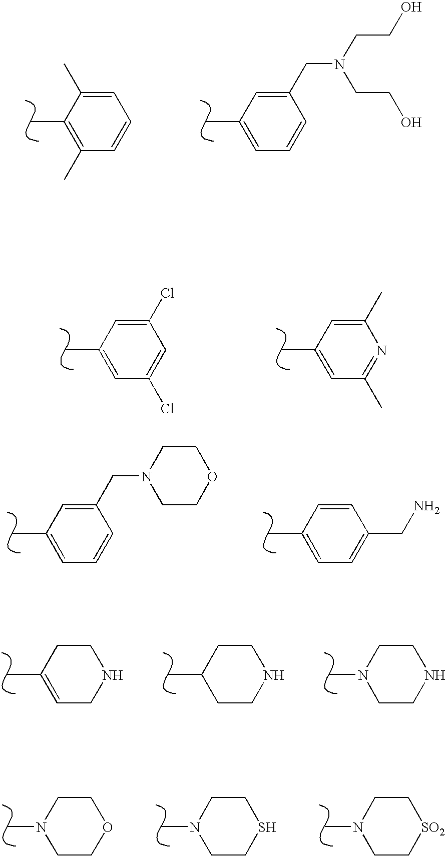
where a wavy line 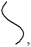
in any orientation, indicates the covalent attachment site of the other structural moieties of the compound.

Exemplary embodiments of C₆-C₂₀ substituted aryl groups include halo-substituted phenyl such as 4-fluorophenyl, 4-chlorophenyl, 3,5-dichlorophenyl, and 3,5-difluorophenyl. Examples of substituted phenyl carbocycles include:

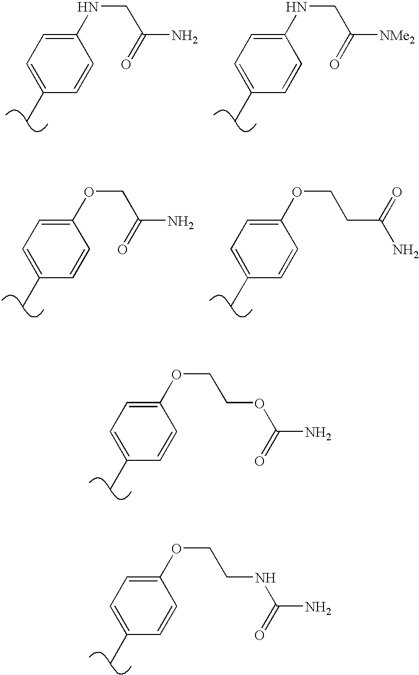

Embodiments of A¹ include: 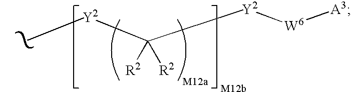
and where one or more Y² are a bond, such as:

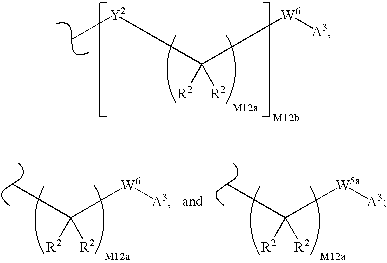

where W^{5a} is a carbocycle or a heterocycle and W^{5a} is independently substituted with 0 or 1 R² groups.

Embodiments of A¹ also include:

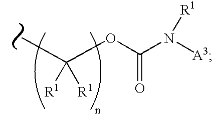

where n is an integer from 1 to 18.

Embodiments of A² include where W³ is W⁵, such as: 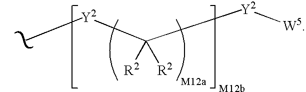
Alternatively, A² is phenyl, substituted phenyl, benzyl, substituted benzyl, pyridyl or substituted pyridyl.

Embodiments of A³ include where M2 is 0, such as: 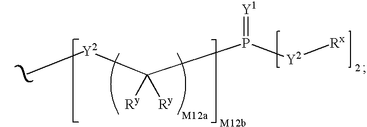
and where M12b is 1, Y¹ is oxygen, and Y^{2b} is independently oxygen (O) or nitrogen (N(R^{x})) such as:

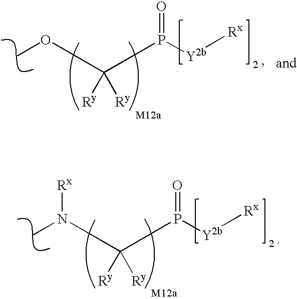

An embodiment of A³ includes: 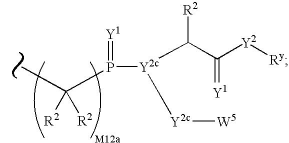
where W⁵ is a carbocycle such as phenyl or substituted phenyl, and Y^{2c} is independently O, N(R^{y}) or S. For example, R² may be H and M12a may be 1.

An embodiment of A³ includes: 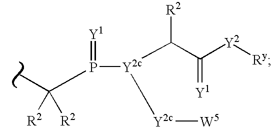
where Y^{2c} is O, N(R^{y}) or S. For example, R¹ may be H and n may be 1.

Another embodiment of A³ includes: 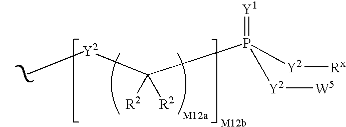
where W⁵ is a carbocycle such as phenyl or substituted phenyl.

Embodiments of R^{x} include esters, carbamates, carbonates, thioesters, amides, thioamides, and urea groups:

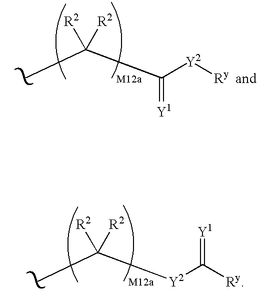

Such embodiments of A³ include: 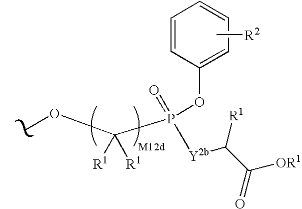
where Y^{2b} is O or N(R^{x}); M12d is 1, 2, 3, 4, 5, 6, 7 or 8; and the phenyl carbocycle is substituted with 0 to 3 R² groups. Such embodiments of A³ include phenyl phosphonamidate amino acid, e.g. alanate esters and phenyl phosphonate-lactate esters:

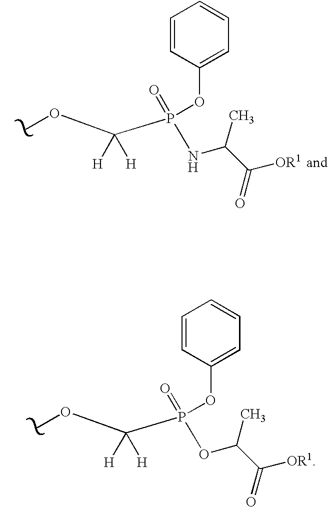

The chiral carbon of the amino acid and lactate moieties may be either the R or S configuration, such as:

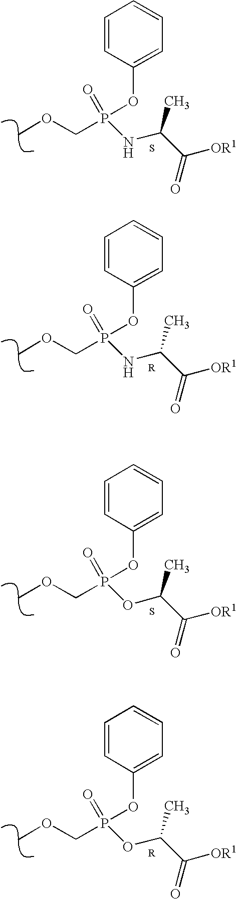

The compounds including amino acid and lactate moieties may alternatively exist as enantiomerically-enriched mixtures or as racemic mixtures.

Formula I-XXXIX compounds include all pharmaceutically acceptable salts thereof. Formula I-XXXIX compounds also include all enol, tautomeric, and resonance isomers, enantiomers, diastereomers, and racemic mixtures thereof.

Phosphonate groups of the compounds of the invention may comprise the substituent structure A³.

The compounds of the invention include one or more phosphonate groups located as a covalently-attached substituent at any location of Formulas I-XXXIX. Prodrug moieties of phosphorus functionality may serve to mask anionic charges and decrease polarity. The phosphonate prodrug moiety may be an ester (Oliyai et al Pharmaceutical Res. (1999) 16:1687-1693; Krise, J. and Stella, V. Adv. Drug Del. Reviews (1996) 19:287-310; Bischofberger et al, U.S. Pat. No. 5,798,340; Oliyai, et al Intl. Jour. Pharmaceutics (1999) 179:257-265), e.g. POC and POM (pivaloyloxymethyl, Yuan, et al Pharmaceutical Res. (2000) 17:1098-1103), or amidate which separates from the integrase inhibitor compound in vivo or by exposure in vitro to biological conditions, e.g. cells, tissue isolates. The separation may be mediated by general hydrolytic conditions, oxidation, enzymatic action or a combination of steps.

Compounds of the invention bearing one or more phosphonate groups may increase or optimize the bioavailability of the compounds as therapeutic agents. For example, bioavailability after oral administration may be preferred and depend on resistance to metabolic degradation in the gastrointestinal tract or circulatory system, and eventual uptake inside cells. Prodrug moieties are considered to confer said resistance by slowing certain hydrolytic or enzymatic metabolic processes. Lipophilic prodrug moieties may also increase active or passive transport of the compounds of the invention across cellular membranes (Darby, G. Antiviral Chem. & Chemotherapy (1995) Supp. 1, 6:54-63).

In one aspect, the compounds of the invention include an active form for inhibition of nuclear integration of reverse-transcribed HIV DNA.

Exemplary embodiments of the invention includes phosphonamidate and phosphoramidate (collectively “amidate”) prodrug compounds. General formulas for phosphonamidate and phosphoramidate prodrug moieties include:

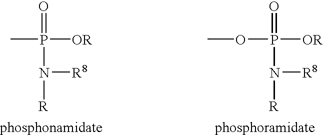

The phosphorus atom of the phosphonamidate group is bonded to a carbon atom. The nitrogen substituent R⁸ may include an ester, an amide, or a carbamate functional group. For example, R⁸ may be —CR₂C(═O)OR′ where R′ is H, C₁-C₆ alkyl, C₁-C₆ substituted alkyl, C₆-C₂₀ aryl, C₆-C₂₀ substituted aryl, C₂-C₂₀ heterocycle, or C₂-C₂₀ substituted heterocycle. The nitrogen atom may comprise an amino acid residue within the prodrug moiety, such as a glycine, alanine, or valine ester (e.g. valacyclovir, see: Beauchamp, et al Antiviral Chem. Chemotherapy (1992) 3:157-164), such as the general structure: 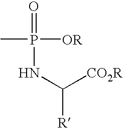
where R′ is the amino acid side-chain, e.g. H, CH₃, CH(CH₃)₂, etc.

An exemplary embodiment of a phosphonamidate prodrug moiety is:

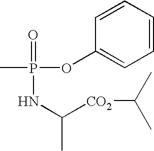

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

Recursive Substituents

Selected substituents within the compounds of the invention are present to a recursive degree. In this context, “recursive substituent” means that a substituent may recite another instance of itself. Because of the recursive nature of such substituents, theoretically, a large number may be present in any given embodiment. For example, R^{x} contains a R^{y} substituent. R^{y} can be R², which in turn can be R³. If R³ is selected to be R^{3c}, then a second instance of R^{x} can be selected. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

By way of example and not limitation, W³, R^{y} and R³ are all recursive substituents in certain embodiments. Typically, each of these may independently occur 20, 19, 18, 17, 16, 15, 14, 13, 12, 11, 10, 9, 8, 7, 6, 5, 4, 3, 2, 1, or 0, times in a given embodiment. More typically, each of these may independently occur 12 or fewer times in a given embodiment. More typically yet, W³ will occur 0 to 8 times, R^{y} will occur 0 to 6 times and R³ will occur 0 to 10 times in a given embodiment. Even more typically, W³ will occur 0 to 6 times, R^{y} will occur 0 to 4 times and R³ will occur 0 to 8 times in a given embodiment.

Recursive substituents are an intended aspect of the invention. One of ordinary skill in the art of medicinal chemistry understands the versatility of such substituents. To the degree that recursive substituents are present in an embodiment of the invention, the total number will be determined as set forth above.

Group I

In one aspect, the invention includes tricyclic phosphonate Group I compounds represented by the following structure, Formula I: 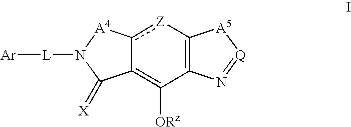
wherein:

A⁴ and A⁵ are each and independently any moiety forming a five, six, or seven membered ring. A⁴ and A⁵ may be independently selected from O, S, NR, C(R²)₂, CR²OR, CR²OC(═O)R, C(═O), C(═S), CR²SR, C(═NR), C(R²)₂—C(R³)₂, C(R²)═C(R³), C(R²)₂—O, NR—C(R³)₂, N═C(R³), N═N, SO₂—NR, C(═O)C(R³)₂, C(═O)NR, C(R²)₂—C(R³)₂—C(R³)₂, C(R²)═C(R³)—C(R³)₂, C(R²)C(═O)NR, C(R²)C(═S)NR, C(R²)═N—C(R³)₂, C(R²)═N—NR, and N═C(R³)—NR. When taken together on a single carbon, two R² or two R³ may form a spiro ring.

Q is N, ⁺NR, or CR⁴.

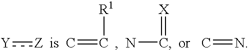

X may be O, S, NH, NR, N—OR, N—NR₂, N—CR₂OR or N—CR₂NR₂.

R^{z} is H; a protecting group selected from benzyhydryl (CHPh₂), trialkylsilyl (R₃Si), 2-trimethylsiloxyethyl, alkoxymethyl (CH₂OR), and ester (C(═O)R); or a prodrug moiety;

R¹, R², R³ and R⁴ are each independently selected from H, F, Cl, Br, I, OH, —NH₂, —NH₃⁺, —NHR, —NR₂, —NR₃⁺, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, —SO₂R, —SO₂Ar, —SOAr, —SAr, —SO₂NR₂, —SOR, —CO₂R, —C(═O)NR₂, 5-7 membered ring lactam, 5-7 membered ring lactone, —CN, —N₃, —NO₂, C₁-C₈ alkoxy, C₁-C₈ trifluoroalkyl, C₁-C₈ alkyl, C₁-C₈ substituted alkyl, C₃-C₁₂ carbocycle, C₃-C₁₂ substituted carbocycle, C₆-C₂₀ aryl, C₆-C₂₀ substituted aryl, C₂-C₂₀ heteroaryl, and C₂-C₂₀ substituted heteroaryl, polyethyleneoxy, phosphonate, phosphate, and a prodrug moiety;

when taken together on a single carbon, two R² or two R³ may form a spiro ring; and

R¹, R², R³, and R⁴ also include: —OC(═O)OR, —OC(═O)NR₂, —OC(═S)NR₂, —OC(═O)NRNR₂, —OC(═O)R, —C(═O)OR, —C(═O)NR₂, —C(═O)NRNR₂, —C(═O)R, —OSO₂NR₂ (sulfamate), —NR₂, —NRSO₂R, —NRC(═S)NR₂, —SR, —S(O)R, —SO₂R, —SO₂NR₂ (sulfonamide), —OSO₂R (sulfonate), —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof.

R may be independently selected from H, C₁-C₈ alkyl, C₁-C₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heteroaryl, C₂-C₂₀ substituted heteroaryl, polyethyleneoxy, phosphonate, phosphate, and a prodrug moiety. Two R groups may form a ring, such as when the two R groups are bonded to a nitrogen atom and form a ring such as aziridinyl, azetidinyl, pyrrolidinyl, pyrazinyl, imidazolyl, piperidyl, piperazinyl, pyridinium, or morpholino.

Exemplary embodiments of R¹, R², R³, and R⁴ include the structures: 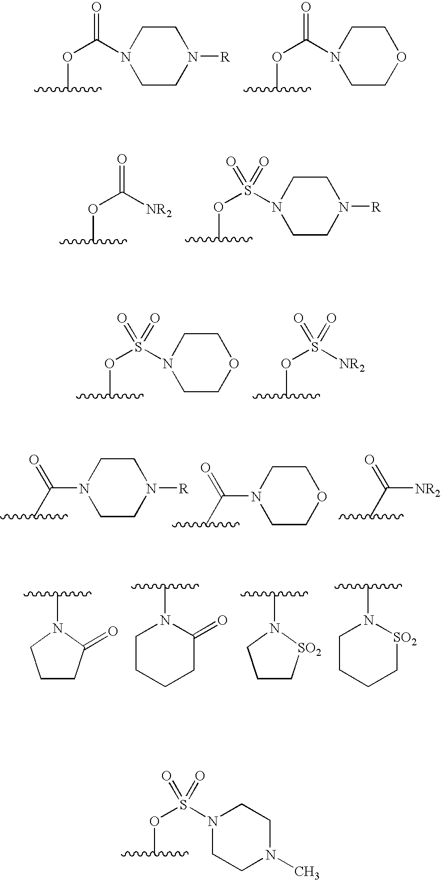
where the wavy line indicates the point of covalent attachment on the tricyclic structure.

Alternatively, R, R¹, R², R³, or R⁴ may independently comprise A¹, A³ or L-A³.

L is a bond or any linker which covalently attaches the Ar group to the tricyclic scaffold. For example, L may be a bond, O, S, S—S (disulfide), S(═O) (sulfoxide), S(═O)₂ (sulfone), S(═O)₂NR (sulfonamide), NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, —(CR₂)ₙO(CR₂)ₙ—, C(═O)NH, OC(═O)NH, NHC(═O)NH, C(═O), C(═O)NH(CH₂)ₙ, or (CH₂CH₂O)ₙ, where n may be 1, 2, 3, 4, 5, or 6.

Substituted alkylene, substituted alkyenylene, substituted alkynylene, substituted aryl, and substituted heteroaryl are independently substituted with one or more substituents selected from F, Cl, Br, I, OH, amino (—NH₂), ammonium (—NH₃⁺), alkylamino, dialkylamino, trialkylammonium, C₁-C₈ alkyl, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—CO₂R), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heteroaryl, and C₂-C₂₀ substituted heteroaryl, phosphonate, phosphate, polyethyleneoxy, and a prodrug moiety.

Ar groups may be any saturated, unsaturated or aromatic ring or ring system comprising a mono- or bicyclic carbocycle or heterocycle, e.g. 3 to 12 ring atoms. The rings are saturated when containing 3 ring atoms, saturated or mono-unsaturated when containing 4 ring atoms, saturated, or mono- or di-unsaturated when containing 5 ring atoms, and saturated, mono- or di-unsaturated, or aromatic when containing 6 ring atoms.

For example, Ar may be C₃-C₁₂ carbocycle, C₃-C₁₂ substituted carbocycle, C₆-C₂₀ aryl, C₆-C₂₀ substituted aryl, C₂-C₂₀ heteroaryl, or C₂-C₂₀ substituted heteroaryl.

Exemplary embodiments of C₆-C₂₀ substituted aryl groups include halo-substituted phenyl such as 4-fluorophenyl, 4-chlorophenyl, 4-trifluoromethyl, 2-amide phenyl, 3,5-dichlorophenyl, and 3,5-difluorophenyl.

Ar groups include substituted phenyl groups such as, but not limited to:

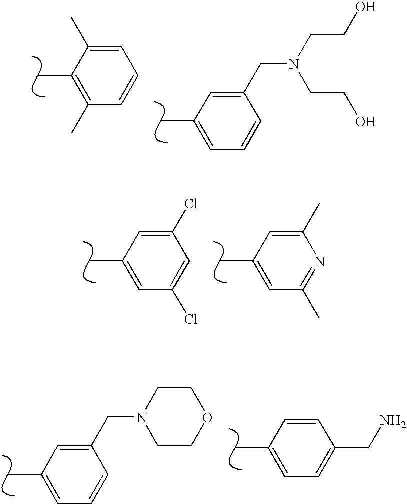

Other examples of substituted phenyl groups include: 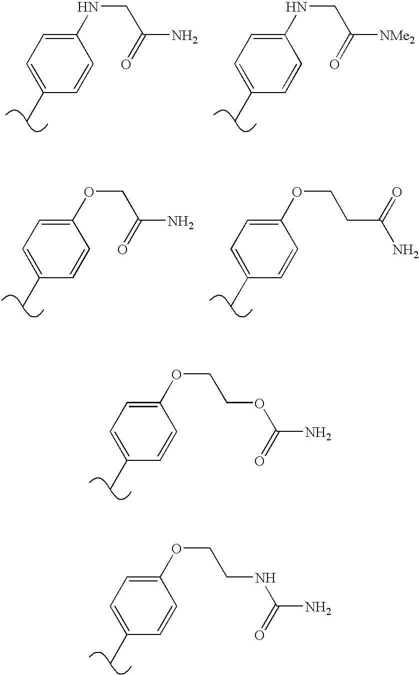
where a wavy line 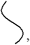
in any orientation, indicates the covalent attachment site to L.

Ar groups also include disubstituted phenyl groups such as, but not limited to: 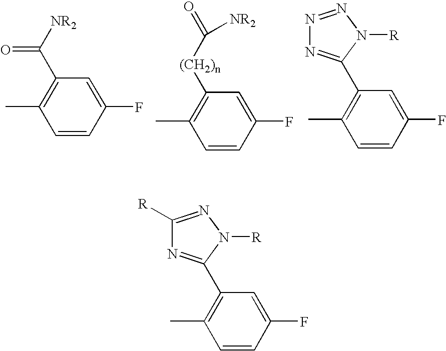
where n is 1 to 6.

Ar groups also include carbocycles such as, but not limited to:

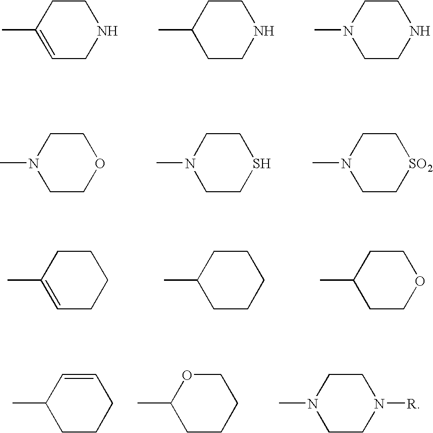

Ar groups also include phenyl and substituted phenyl fused to a carbocycle to form groups including:

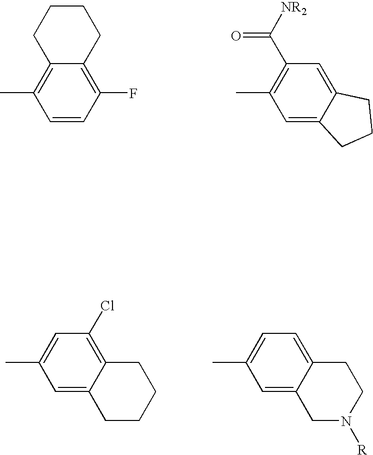

Substituents of Ar, may independently be H, F, Cl, Br, I, OH, amino (—NH₂), ammonium (—NH₃⁺), alkylamino, dialkylamino, trialkylammonium, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—CO₂R), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ trifluoroalkyl, C₁-C₈ alkyl, C₁-C₈ substituted alkyl, C₃-C₁₂ carbocycle, C₃-C₁₂ substituted carbocycle, C₆-C₂₀ aryl, C₆-C₂₀ substituted aryl, C₂-C₂₀ heteroaryl, and C₂-C₂₀ substituted heteroaryl, phosphonate, phosphate, polyethyleneoxy, and a prodrug moiety.

The following embodiments of A⁴ and A⁵ in Formula I compounds include but are not limited to the following structures. Various embodiments of A⁴ form 5-membered rings in the exemplary structures:

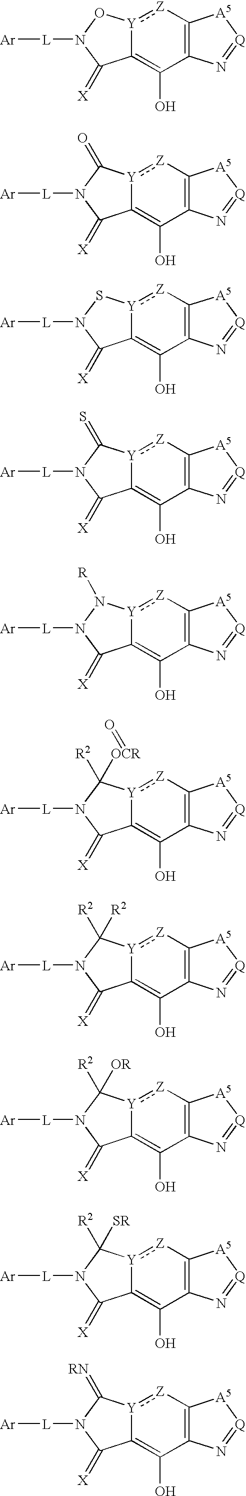

Various embodiments of A⁴ form 6-membered rings in the exemplary structures:

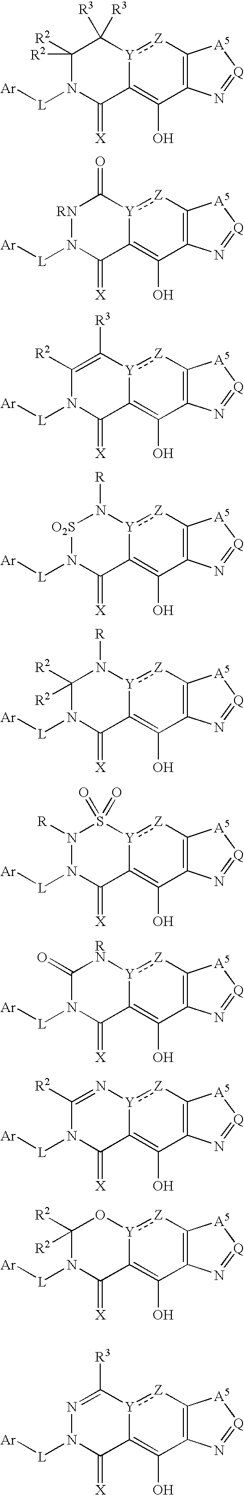

Various embodiments of A⁴ form 7-membered rings in the exemplary structures:

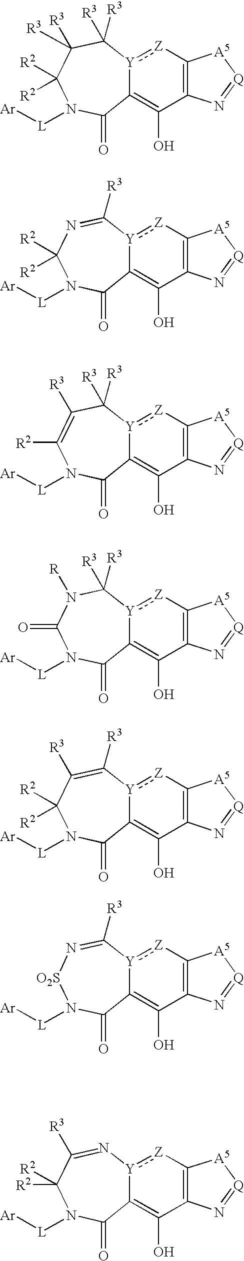

Various embodiments of A⁵ form 5-membered rings in the exemplary structures:

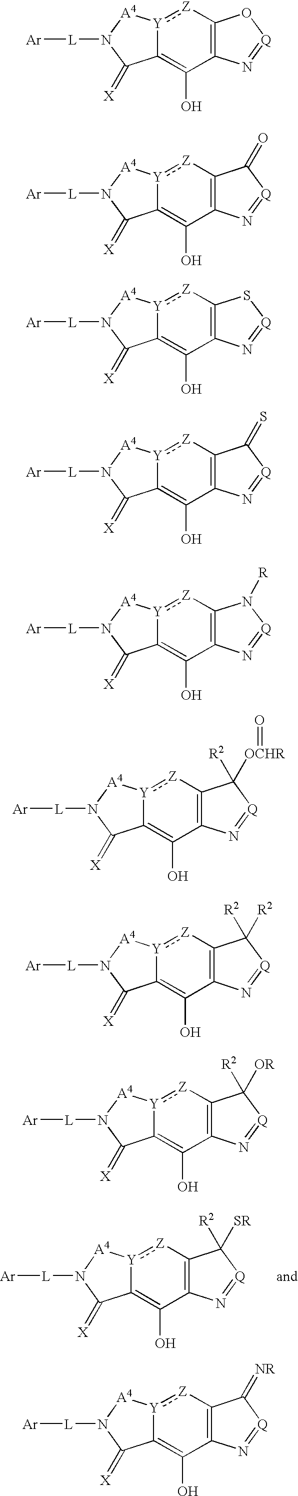

Other various embodiments of A⁵ form 6-membered rings in the exemplary structures:

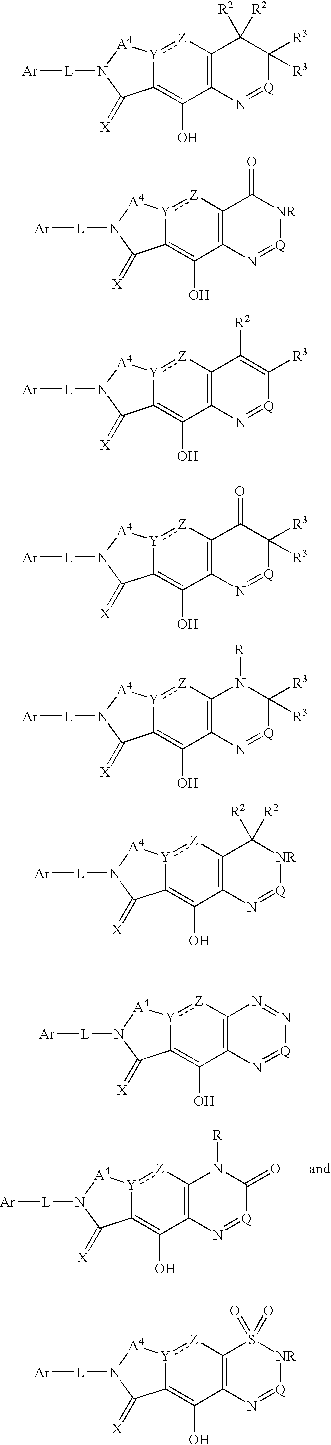

Other various embodiments of A⁵ form 7-membered rings in the exemplary structures:

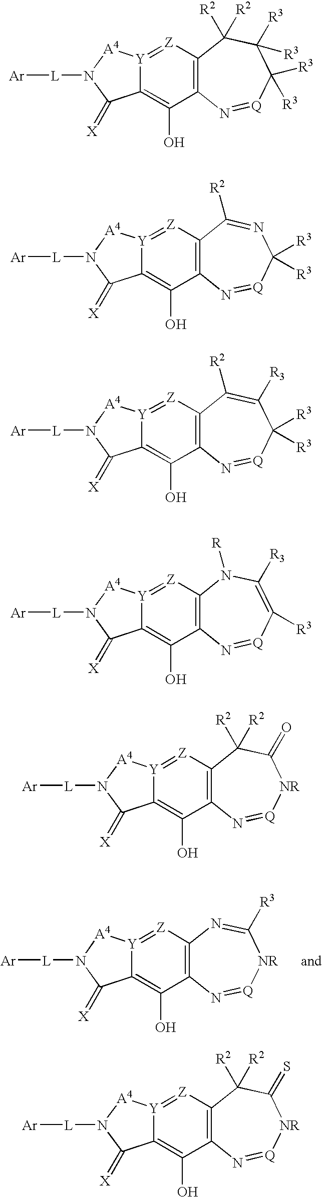

Formula I compounds of the invention include the following structures:

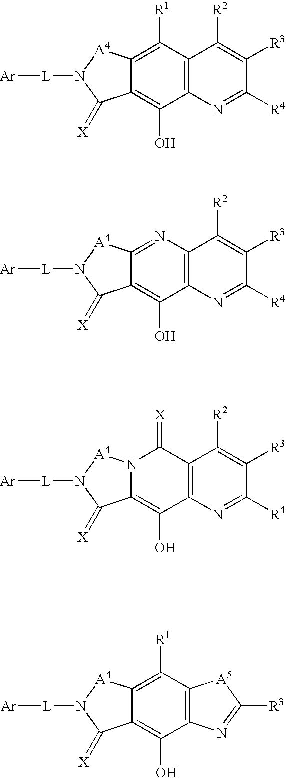

Formula I compounds thus include the following succinimide structure:

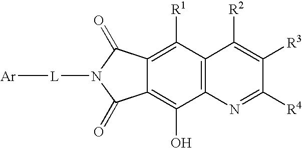

Embodiments of Formula I also include Ia-c where A⁴ is CH₂, CH₂CH₂, and CH₂CH₂CH₂, respectively:

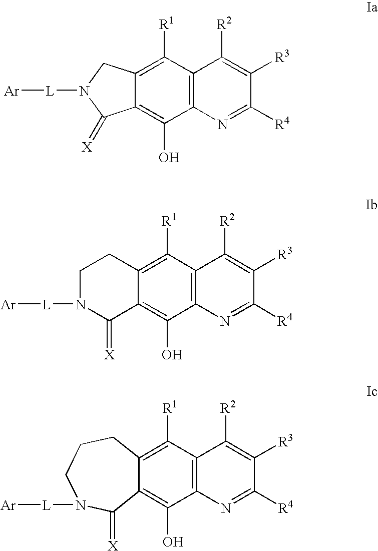

Where A⁴ forms a seven-membered ring, the 7 membered ring may be comprised of a second amide group, as shown by exemplary Formula Id:

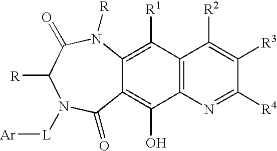

One aspect of the invention includes compounds with a cyclic imide group, e.g. 5,9-dihydroxy-pyrrolo[3,4-g]quinoline-6,8-dione (Myers, et al U.S. Pat. No. 5,252,560; Robinson, U.S. Pat. No. 5,854,275), where A is C(═O) and X is O, as in formula Ie:

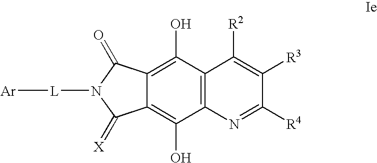

Along with other compounds of the invention, the cyclic imide group of Formula Ie provides functionality which may be in a pre-organized state for optimized HIV integrase inhibition relative to compounds without the cyclic imide group (Anthony, et al WO 02/30931; Zhuang, et al “Design and synthesis of 8-hydroxy-1,6-naphthyridines as novel HV-1 integrase inhibitors” Interscience Conference on Antimicrobial Agents and Chemotherapy, San Diego, Calif., Sep. 27-30, 2002).

Formula Ia compounds include the following amide structure: 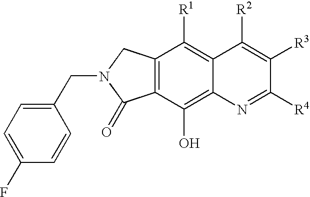
Group II

In one aspect, the invention includes phosphonate analogs of aza-quinolinol compounds (Zhuang et al (2003) J. Med. Chem. 46(4):453-456; Zouhiri et al (2000) J. Med. Chem. 43(8):1533-1540; Ouali et al (2000) J. Med. Chem. 43(10)1949-1957; d'Angelo et al (2001) Pathol. Biol. 49:237-246; Mekouar et al (1998) J. Med. Chem. 41:2846-2857; WO 03/62204; WO 03/016315 A1; WO 03/016309 A1; WO 03/016294 A1; WO02/070486 A1; WO 02/055079; WO 02/030930; WO 02/030931; WO 02/30426) represented by the Formula II: 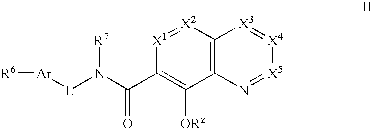
wherein:

X¹ is CR¹, NR, or N;

X² is CR², NR, or N;

X³ is CR³, NR, or N;

X⁴ is CR⁴, NR, or N;

X⁵ is CR⁵, NR, or N;

at least one of X¹, X², X³, X⁴, and X⁵ is NR or N;

R¹, R², R³, R⁴, R⁶, R⁶, and R⁷ are independently selected from H, F, Cl, Br, I, OH, amino (—NH₂), ammonium (—NH₃⁺), alkylamino, dialkylamino, trialkylammonium, C₁-C₈ alkyl, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—C(═O)OR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heteroaryl, and C₂-C₂₀ substituted heteroaryl, phosphonate, phosphate, polyethyleneoxy, and a prodrug moiety.

R is independently selected from H, C₁-C₈ alkyl, C₁-C₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heteroaryl, and C₂-C₂₀ substituted heteroaryl.

Alternatively, R, R¹, R², R³, R⁴, R⁵, R⁶, or R⁷ may independently comprise A¹, A³ or L-A³.

At least one of R, R¹, R², R³, R⁴, R⁵, R⁶, and R⁷ comprises a phosphonate group. The phosphonate group may be a prodrug moiety. The phosphonate group may be directly attached to a ring carbon (CR¹, CR², CR³, CR⁴ or CR⁵) of Formula II.

R^{z} is H; a protecting group selected from benzyhydryl (CHPh₂), trialkylsilyl (R₃Si), 2-trimethylsiloxyethyl, alkoxymethyl (CH₂OR), and ester (C(═O)R); or a prodrug moiety;

L is a bond or any linker which covalently attaches the Ar group to the tricyclic scaffold. For example, L may be a bond, O, S, S(═O) (sulfoxide), S(═O)₂ (sulfone), S(═O)₂NR (sulfonamide), N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C(═O)NH, C(═O), C(═O)NH(CH₂)ₙ, or (CH₂CH₂O)ₙ, where n may be 1, 2, 3, 4, 5, or 6.

Ar groups may be any saturated, unsaturated or aromatic ring or ring system comprising a mono- or bicyclic carbocycle or heterocycle, e.g. 3 to 10 ring atoms. The rings are saturated when containing 3 ring atoms, saturated or mono-unsaturated when containing 4 ring atoms, saturated, or mono- or di-unsaturated when containing 5 ring atoms, and saturated, mono- or di-unsaturated, or aromatic when containing 6 ring atoms.

Ar is covalently attached to L and to one or more R⁶.

Exemplary structures within Formula II include the following:

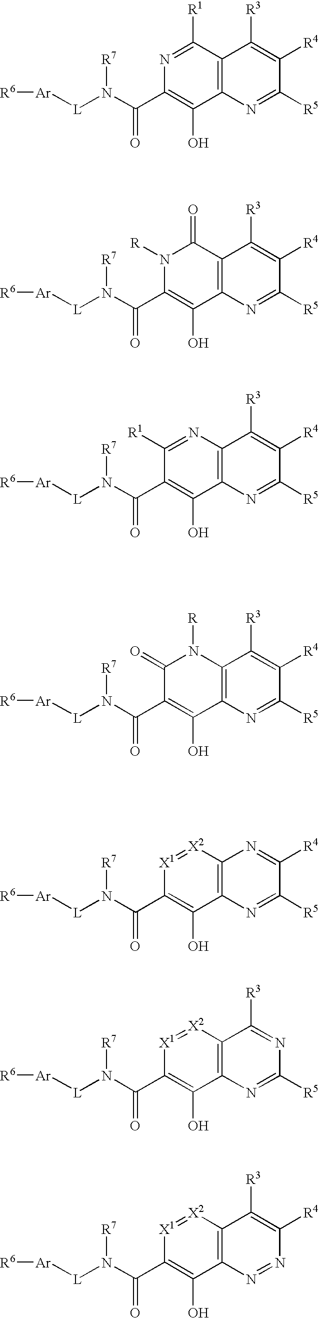

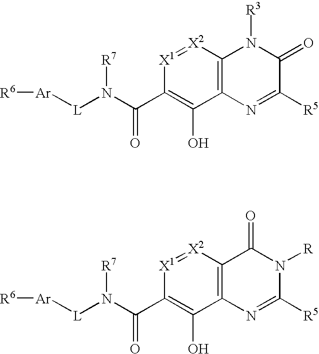

When X¹ is CR¹ and when X² is CR², then CR¹ and CR² together may form a ring. When X³ is CR³ and when X⁴ is CR⁴, then CR³ and CR⁴ together may form a ring. When X⁴ is CR⁴ and X⁵ is CR⁵, then CR⁴ and CR⁵ together may form a ring. The ring may be 5, 6, or 7-membered. The ring may be all carbon atoms or it may have one or more heteroatoms selected from nitrogen, oxygen, and sulfur.

Exemplary structures when CR⁴ and CR⁵ form a ring include the following:

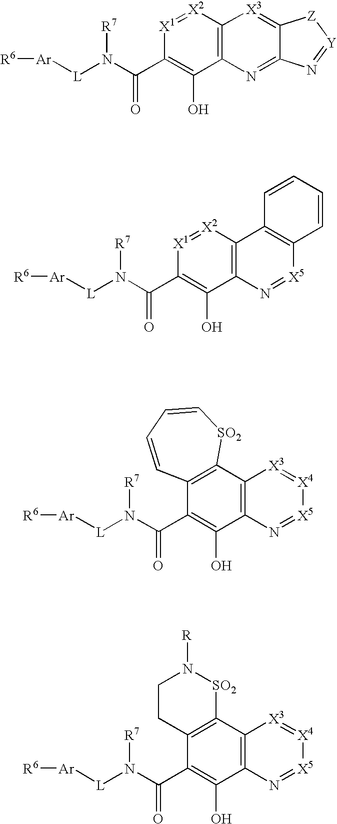

Y is CR⁵, NR or N. Z is a moiety forming a five, six, or seven membered ring. For example, Z may be O, S, NR, CR₂, CROR, CROC(═O)R, C(═O), C(═S), CRSR, C(═NR₂), C═CR₂, CR₂—CR₂, CR═CR, NR—CR₂, N═CR, N═N, SO₂—NR, C(═O)CR₂, S(═O)CR₂, SO₂CR₂, C(═O)NR, CR₂—CR₂—CR₂, CR═CR—CR₂, CRC(═O)NR, CR₂SO₂CR₂, CR₂SO₂NR, CRC(═S)NR, CR═N—CR₂, CR═N—NR, or N═CR—NR.

R² may be —H, —OH, —OC(═O)OR, —OC(═O)NR₂, —OC(═S)NR₂, —OC(═O)NRNR₂, —OC(═O)R, —C(═O)OR, —C(═O)NR₂, —C(═O)NRNR₂, —C(═O)R, —OSO₂NR₂ (sulfamate), —NR₂, —NRSO₂R, —NRC(═S)NR₂, —SR, —S(O)R, —SO₂R, —SO₂NR₂ (sulfonamide), —OSO₂R (sulfonate), —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof.

R² may include a ring, e.g. 4-7 membered ring lactam or sultam, or piperazinyl sulfamate:

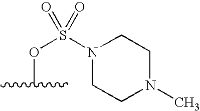

Exemplary embodiments of Formula II compounds include: 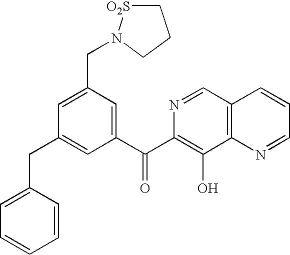
where at least one aryl or sultam ring carbon atom is substituted with an A¹ group, and any aryl or sultam ring carbon atom may be substituted with an A2 group, including the exemplary structures: 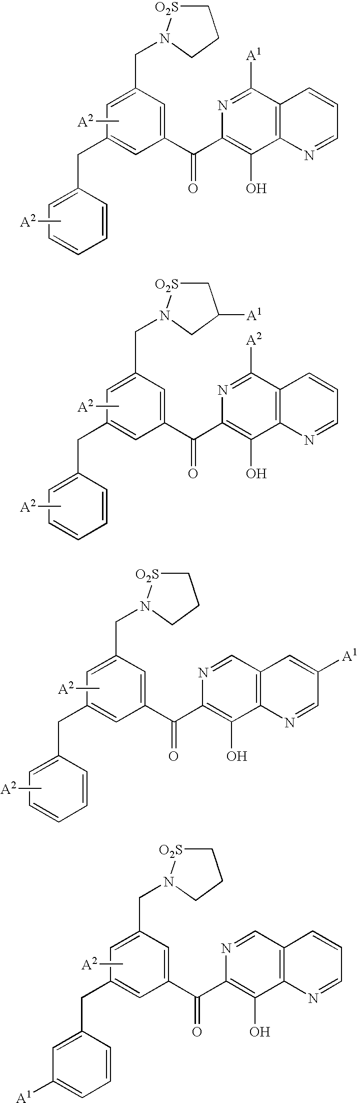
Group III

In one aspect, the invention includes phosphonate analogs of quinoline compounds (WO 03/031413 A1) represented by the Formula III:

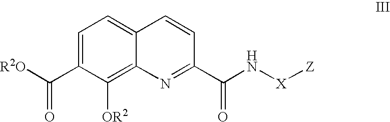

wherein X is L and Z is R⁶—Ar as defined in Formula II. R^{z} is H; a protecting group selected from benzyhydryl (CHPh₂), trialkylsilyl (R₃ Si), 2-trimethylsiloxyethyl, alkoxymethyl (CR₂OR), and ester (C(═O)R); or a prodrug moiety. The aryl carbons and amide nitrogen may be further substituted as defined in the following embodiments of Formula III.

Embodiments of Formula III include the structures:

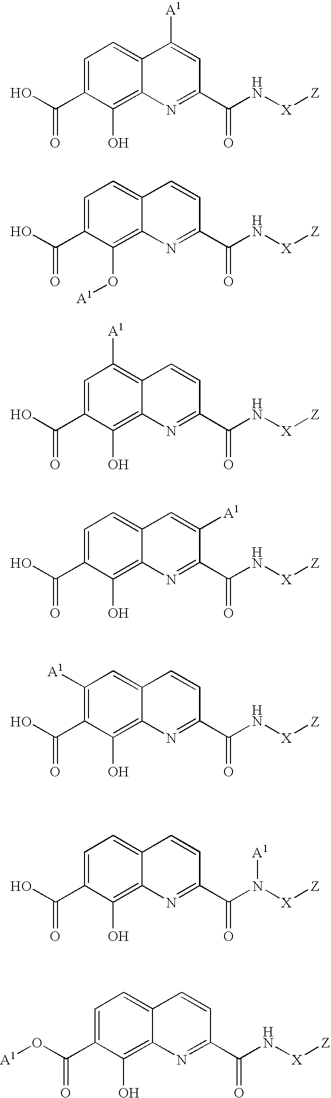

Further embodiments of Formula III compounds include the following: 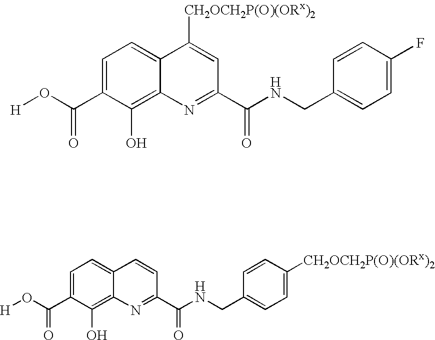
Group IV

In one aspect, the invention includes phosphonate analogs of 4,5-dihydroxypyrimidine, 6-carboxamide compounds (WO 03/035076 A1) having Formula IV: 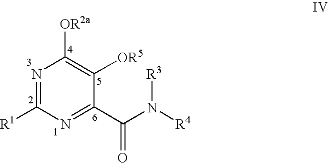
wherein:

R¹ is selected from H, F, Cl, Br, I, OH, OR, amino (—NH₂), ammonium (—NH₃⁺), alkylamino (—NHR), dialkylamino (—NR₂), trialkylammonium (—NR₃⁺), carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, and a prodrug moiety;

R^{2a} and R⁵ are each independently selected from H, sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, and a prodrug moiety;

R^{2b}, R³, and R⁴ are each independently selected from H, OH, OR, amino (—NH₂), ammonium (—NH₃+), alkylamino (—NHR), dialkylamino (—NR₂), trialkylammonium (—NR₃⁺), carboxyl (—CO₂H), sulfate, sulfamate, sulfonate, 5-7 membered ring sultam, 4-dialkylaminopyridinium, alkylsulfone (—SO₂R), arylsulfone (—SO₂Ar), arylsulfoxide (—SOAr), arylthio (—SAr), sulfonamide (—SO₂NR₂), alkylsulfoxide (—SOR), formyl (—CHO), ester (—CO₂R), amido (—C(═O)NR₂), 5-7 membered ring lactam, 5-7 membered ring lactone, nitrile (—CN), azido (—N₃), nitro (—NO₂), C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, a protecting group, and a prodrug moiety;

R is independently selected from H, C₁-C₈ alkyl, C₁-C₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heteroaryl, and C₂-C₂₀ substituted heteroaryl.

Alternatively, R, R¹, R^{2a}, R³, R⁴, or R⁵ may independently comprise A¹, A³ or L-A³.

At least one of R, R¹, R^{2a}, R³, R⁴, and R⁵ comprises a phosphonate group. The phosphonate group may be a prodrug moiety.

Embodiments of R¹, R^{2a}, R^{2b}, R³, R⁴, and R⁵ include —C(═S)NR₂, —C(═O)OR, —C(═O)NR₂, —C(═O)NRNR₂, —C(═O)R, —SO₂NR₂, —NRSO₂R, —NRC(═S)NR₂, —SR, —S(O)R, —SO₂R, —SO₂R, —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof.

Embodiments of R¹, R^{2a}, R^{2b}, R³, R⁴, and R⁵ may also individually or in combination form a ring, e.g. 4-7 membered ring lactam, carbonate, or sultam, or piperazinyl sulfamate:

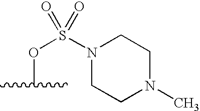

Embodiments of R¹ also include —OC(═S)NR₂, —OC(═O)OR, —OC(═O)NR₂, —OC(═O)NRNR₂, —OC(═O)R, —OP(═O)(OR)₂, —OP(═O)(OR)(NR₂), —OP(═O)(NR₂)₂, —OP(═S)(OR)₂, —OP(═S)(OR)(NR₂), —OP(═S)(NR₂)₂, and including prodrug substituted forms thereof.

A linker may be interposed between positions R¹, R^{2a}, R³, R⁴, or R⁵ and substituent A³, as exemplified in some structures herein as “L-A³”. The linker L may be O, S, NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C(═O)NH, C(═O), S(═O)₂, C(═O)NH(CH₂)ₙ, and (CH₂CH₂O)ₙ, where n may be 1, 2, 3, 4, 5, or 6. Linkers may also be repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide. For example, the linker may comprise propargyl, urea, or alkoxy groups.

Exemplary structures within Formula IV include IVa, IVb, IVc, IVd: 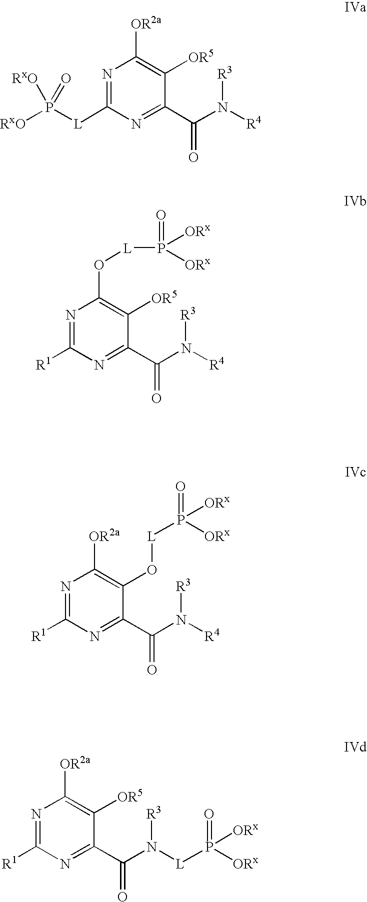
Group V

In one aspect, the invention includes phosphonate analogs of 3-N-substituted, 5-hydroxypyrimidinone, 6-carboxamide compounds (WO 03/035077 A1) having Formula V:

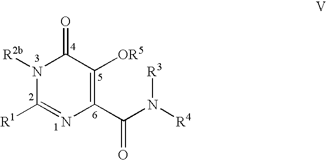

wherein R¹, R^{2b}, R³, R⁴, and R⁵ are as defined for Formula IV. Alternatively, R, R¹, R^{2b}, R³, R⁴, or R⁵ may independently comprise A¹, A³ or L-A³.

At least one of R, R¹, R^{2b}, R³, R⁴, and R⁵ comprises a phosphonate group. The phosphonate group may be a prodrug moiety.

Embodiments of R¹, R^{2b}, R^{2b}, R³, R⁴, and R⁵ include —C(═S)NR₂, —C(═O)OR, —C(═O)NR₂, —C(═O)NRNR₂, —C(═O)R, —SO₂NR₂, —NRSO₂R, —NRC(═S)NR₂, —SR, —S(O)R, —SO₂R, —SO₂R, —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof.

Embodiments of R¹, R^{2a}, R^{2b}, R³, R⁴, and R⁵ may also individually or in combination form a ring, e.g. 4-7 membered ring lactam, carbonate, or sultam, or piperazinyl sulfamate:

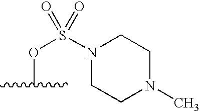

Embodiments of R¹ also include —OC(═S)NR₂, —OC(═O)OR, —OC(═O)NR₂, —OC(═O)NRNR₂, —OC(═O)R, —OP(═O)(OR)₂, —OP(═O)(OR)(NR₂), —OP(═O)(NR₂)₂, —OP(═S)(OR)₂, —OP(═S)(OR)(NR₂), —OP(═S)(NR₂)₂, and including prodrug substituted forms thereof.

A linker may be interposed between positions R¹, R^{2b}, R³, R⁴, or R⁵ and substituent A³, as exemplified in some structures herein as “L-A³”. The linker L may be O, S, NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, C(═O)NH, C(═O), S(═O)₂, C(═O)NH(CH₂)ₙ, and (CH₂CH₂O)ₙ, where n may be 1, 2, 3, 4, 5, or 6. Linkers may also be repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide. For example, the linker may comprise propargyl, urea, or alkoxy groups.

Exemplary structures within Formula V include Va, Vb, Vc, Vd: 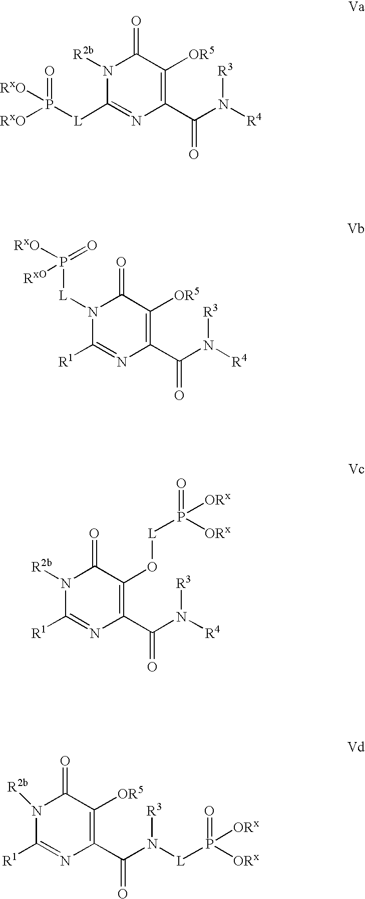
Group VI

In one aspect, the invention includes phosphonate analogs of 1,3 diketo compounds having Formula VI: 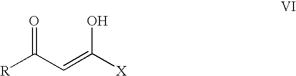
wherein

R is C₁-C₈ alkyl, C₁-C₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heteroaryl, or C₂-C₂₀ substituted heteroaryl (Pais et al (2002) Drugs of the Future 27(11):1101-1111). Alternatively, R may be C₁-C₈ alkylamino, C₁-C₈ substituted alkylamino, C₂-C₁₈ alkenylamino, C₂-C₁₈ substituted alkenylamino, C₂-C₁₈ alkynylamino, C₂-C₁₈ substituted alkynylamino, C₆-C₂₀ arylamino, C₆-C₂₀ substituted arylamino, C₆-C₂₀ arylalkylamino, C₆-C₂₀ substituted arylalkylamino, C₂-C₂₀ heteroarylamino, or C₂-C₂₀ substituted heteroarylamino, whereby the amide is formed (WO 04/004657; WO 01/96283; WO 01/98248). Exemplary Formula VI compounds include where R is benzylamino, thiophenyl, thioimidazolyl, benzothiophenyl, napthothiophenyl, pyrrolidinyl, pyrazolyl, indanyl, indolyl, sesamyl, and benzoxazolyl.

X is: (VIa) a carboxylic acid or ester group (Zhang et al (2003) Bioorganic & Medicinal Chemistry Letters 13(6):1215-1219; Pais et al (2002) Jour. Med. Chem. 45(15):3184-3194; Reinke et al (2002) Antimicrob. Agents and Chemo. 46(10):3301-3303; Marchand et al (2002) Jour. Biological Chem. 277(15):12596-112603; Hazuda et al (2000) Science 287(5453):646-650; Espeseth et al (2000) Proc. Natl. Acad. Sci. USA 97(21):11244-11249; Wai et al (2000) J. Med. Chem. 43(26):2923-2926; U.S. Pat. No. 6,548,546; WO 03/016266 A1; WO 03/049695 A2; WO 03/049690 A2; WO 00/06529, WO 99/62513, WO 01/96283; WO 01/98248); (VIb) a moiety with an acidic proton such as tetrazole or triazole (Pluymers et al (2002) Antimicrob. Agents and Chemo. 46(10):3292-3297; WO 99/50245, WO 00/39086); (VIc) a substituted amide (WO 01/17968, WO 03/16266); (Vd) aryl (WO 99/62520); and (VIe) heteroaryl (WO 01/096329; WO 99/62513, WO 99/62520, WO 99/62897, WO 01/00578).

Embodiments of Formula VI compounds include:

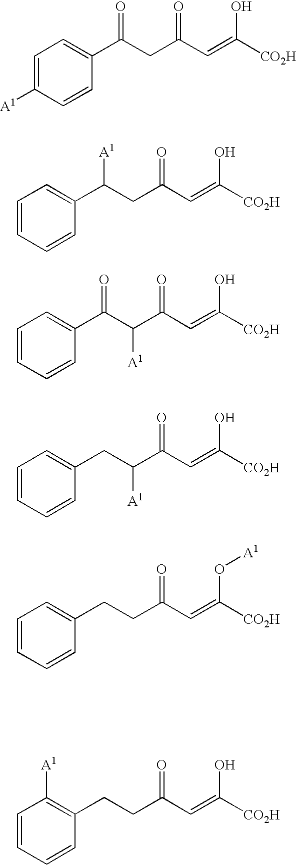

Embodiments of Formula VI compounds also include:

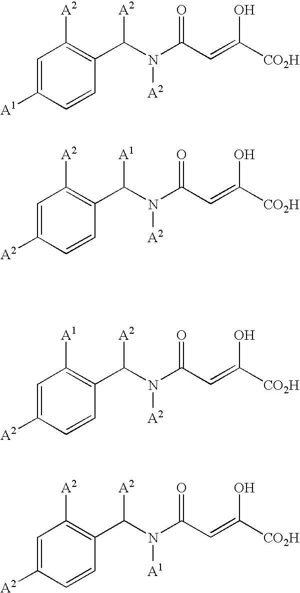

Embodiments of Formula VI compounds also include:

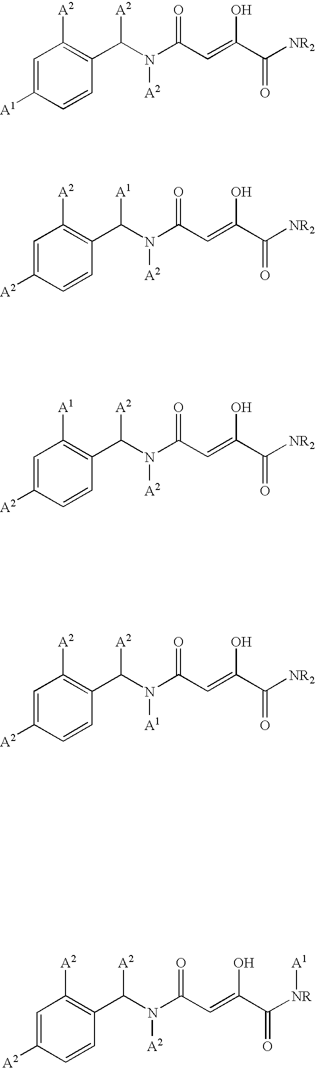

Embodiments of Formula VI compounds also include:

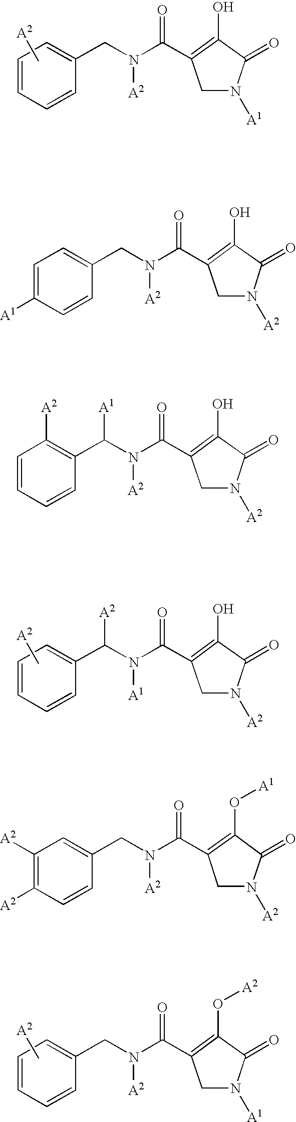

Embodiments of Formula VI compounds also include: 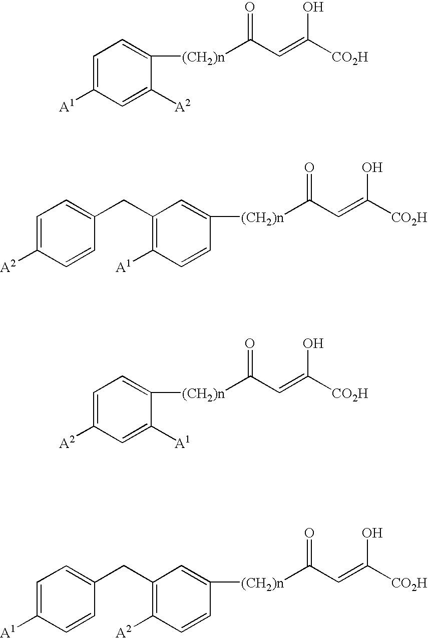
where n may be 1, 2, 3, 4, 5, or 6.

Embodiments of Formula VI compounds also include:

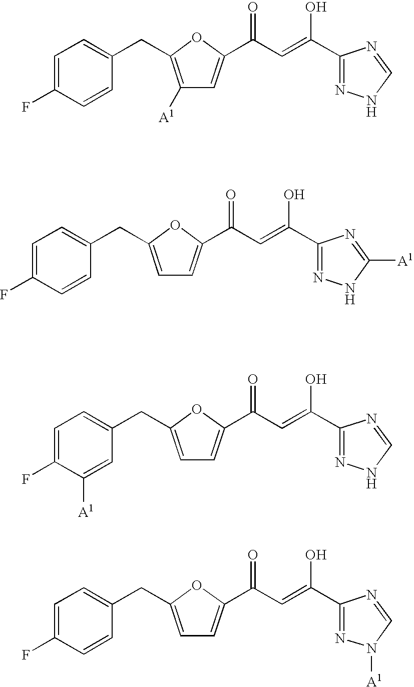

Embodiments of Formula VI compounds also include:

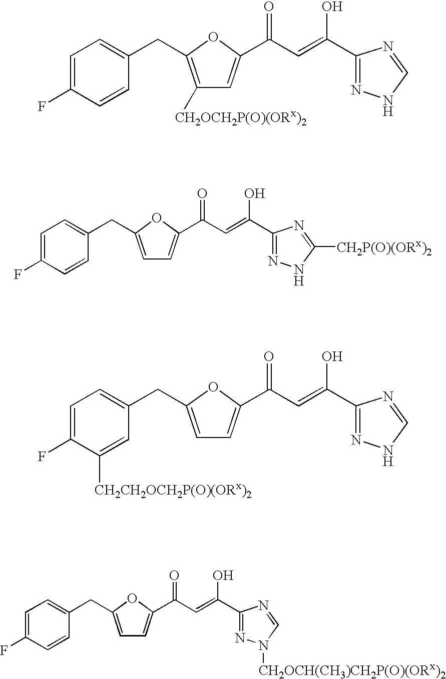

Embodiments of Formula VI compounds also include:

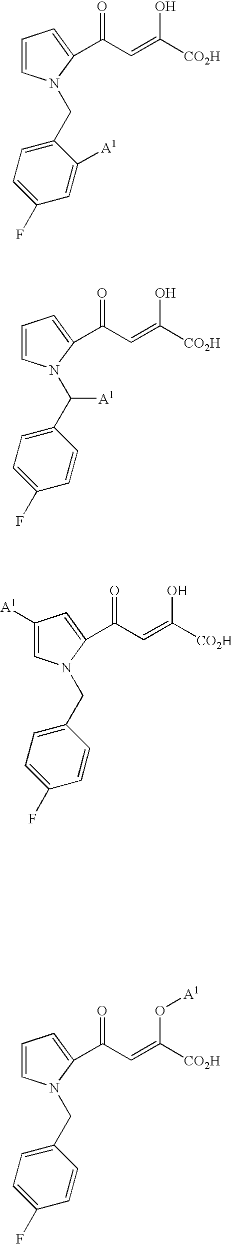

Embodiments of Formula VI compounds also include:

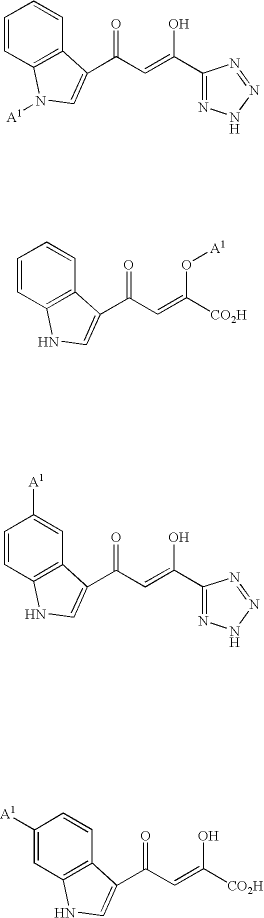

Embodiments of Formula VI compounds also include:

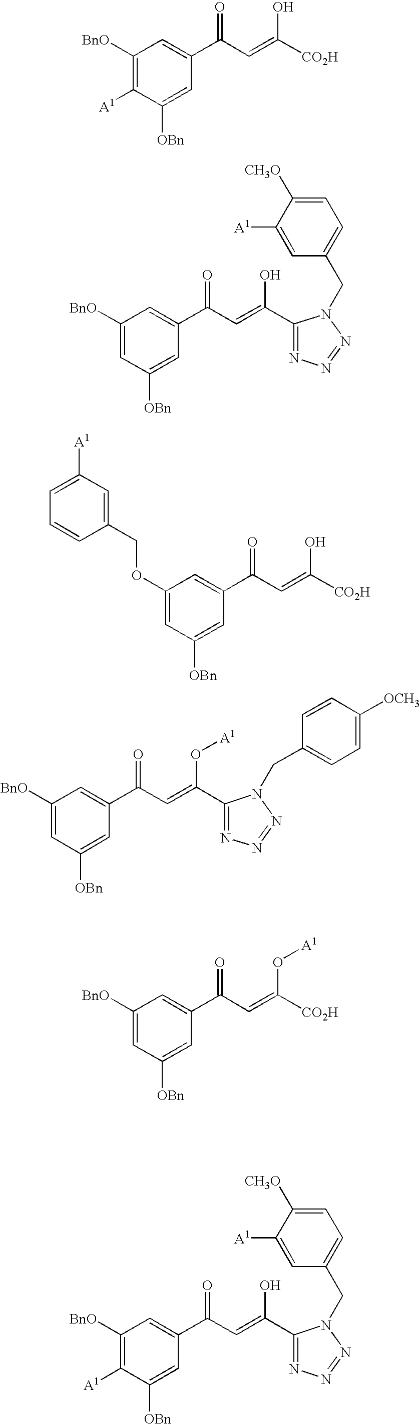

Embodiments of Formula VI compounds also include: 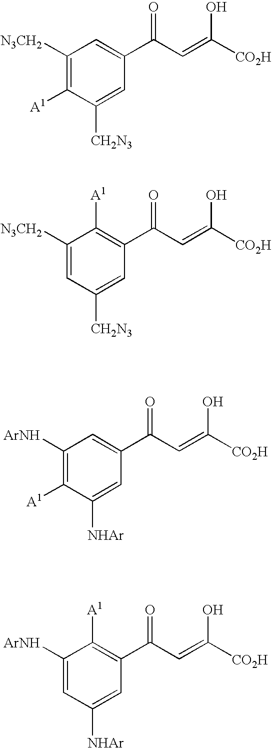
Group VII

In one aspect, the invention includes phosphonate analogs of 2,5 diarylsubstituted, furan compounds having Formula VII:

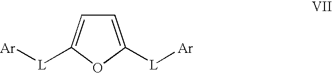

Embodiments of Formula VII compounds include:

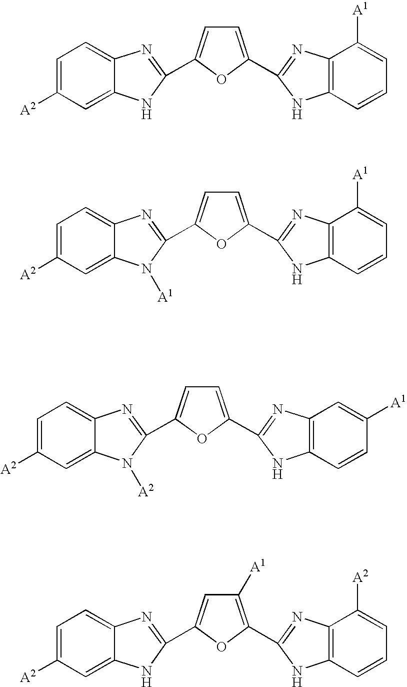

Embodiments of Formula VII compounds also include:

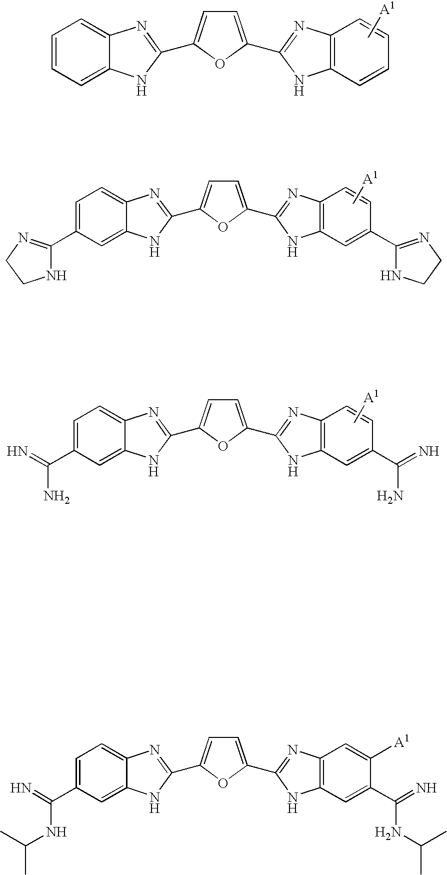

Further embodiments of Formula VII compounds include: 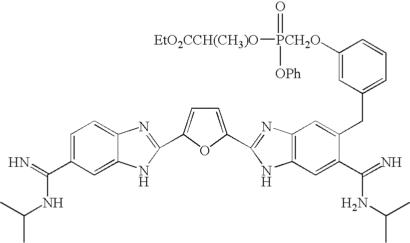
Group VIII

In one aspect, the invention includes phosphonate analogs of 2,5 substituted, diketo-furan compounds (WO 03/016275 A1) having Formula VIII:

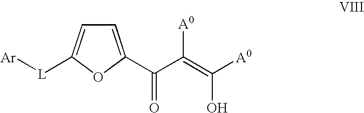

Embodiments of Formula VIII include the structures:

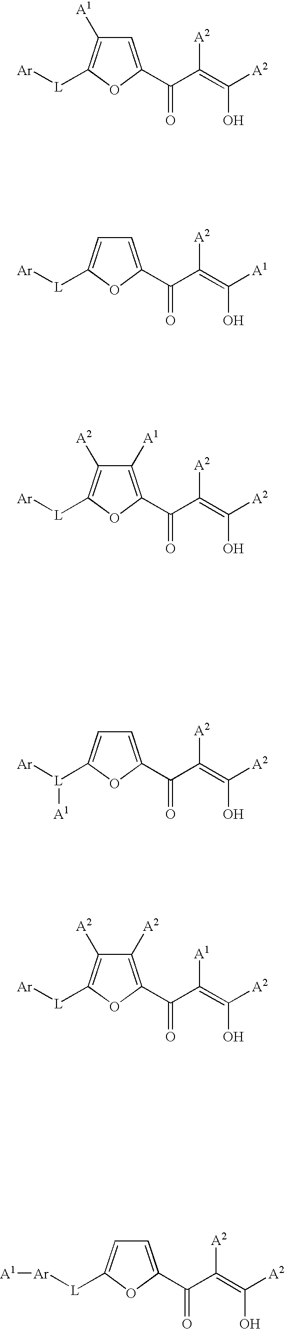

Embodiments of Formula VIII also include the structures:

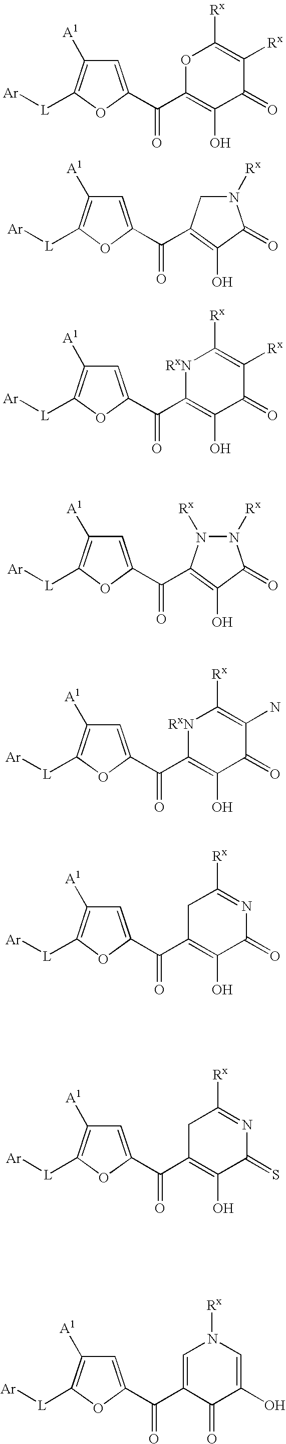

Embodiments of Formula VIII also include the structures: 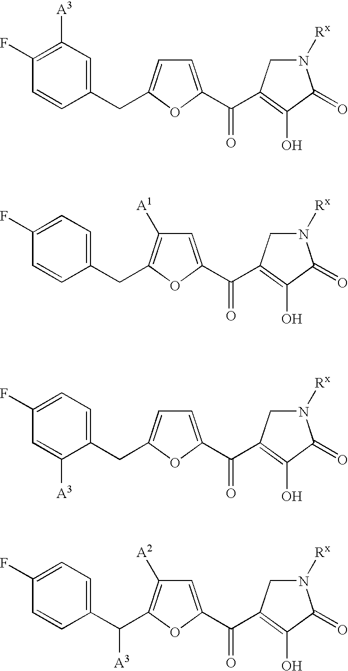
Group IX

In one aspect, the invention includes phosphonate analogs of catechol compounds (Dupont et al (2001) Bioorganic & Medicinal Chemistry Letters 11(24):3175-3178; Neamati et al (1997) Drug Discovery Today 2:487-498; Neamati et al (2000) Adv. Pharmacol. 49:147-165; Fesen et al (1993) Proc. Natl. Acad. Sci USA 90:2399-2403; Lafemina et al (1995) Antimicrob. Agents Chemother. 39:320-324; Eich et al (1996) J. Med. Chem. 39(1):86-95; Pommier et al (1997) Antiviral Chem. Chemother. 8:463-483; Pommier et al (1999) Adv. Virus Res. 52:427-458; Fesen et al (1994) Biochem. Pharmacol. 48:595-608; McDougall et al (1998) Antimicrob. Agents Chemother. 42:140-146; Mazumder et al (1995) Biochem. Pharmacol. 49:1165-1170; Pfeifer et al (1992) J. Pharm. Med. 2:75-97; Mazumder et al (1997) J. Med. Chem. 40:3057-3063; Zhao et al (1997) J. Med. Chem. 40:1186-1194; Neamati et al (1997) Antimicrob. Agents Chemother. 41:385-393; Neamati et al (1997) Mol. Pharmacol. 52:1041-1055; Molteni et al (2000) J. Med. Chem. 43(10):2031-2039; U.S. Pat. No. 6,362,165; GB 2271566), including caffeic acid phenylethyl ester (CAPE) compounds (Patil et al (2003) Abstracts of Papers, 226th ACS National Meeting, New York, N.Y., United States, Sep. 7-11, 2003; having Formula IX:

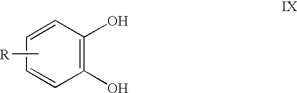

where R is a variety of scaffolds that is covalently attached to the catechol moiety through a single bond or a fused ring system.

Embodiments of Formula IX include the structures:

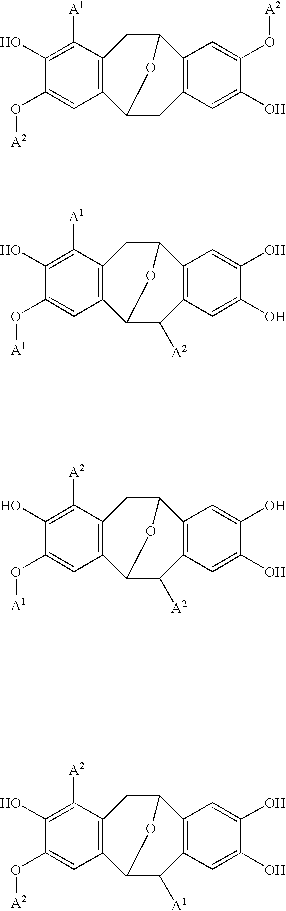

Embodiments of Formula IX also include the structures:

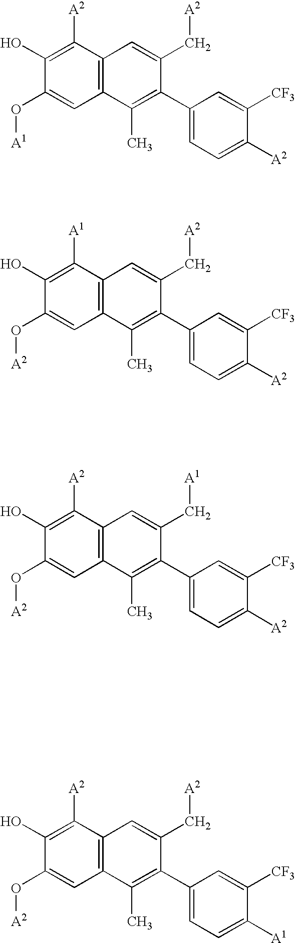

Embodiments of Formula IX also include the structures:

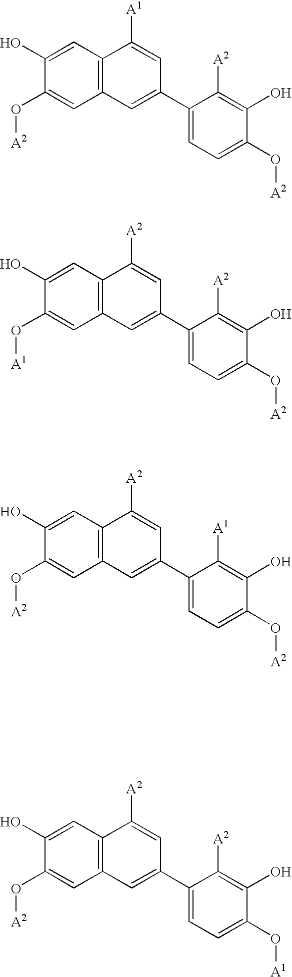

Embodiments of Formula IX also include the dopamine phosponate structures: 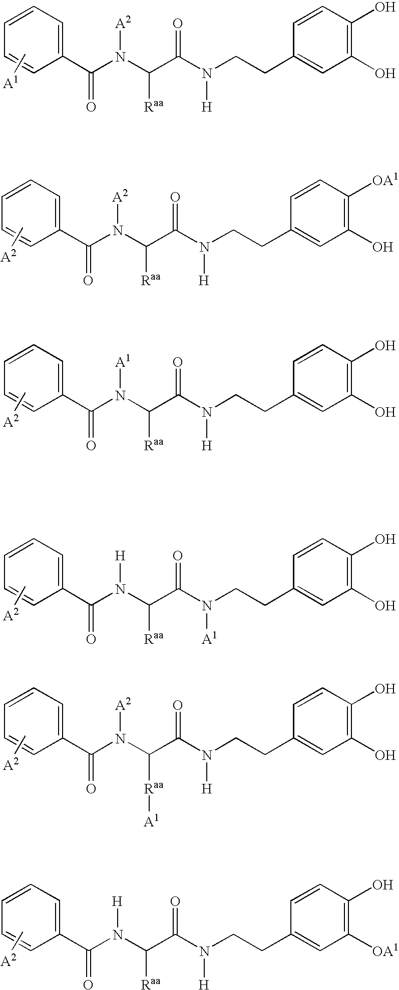
where R^{aa} is an amino acid side chain, including proline.

Embodiments of Formula IX also include the bis catechol, β-conidendrol phosphonate structures: 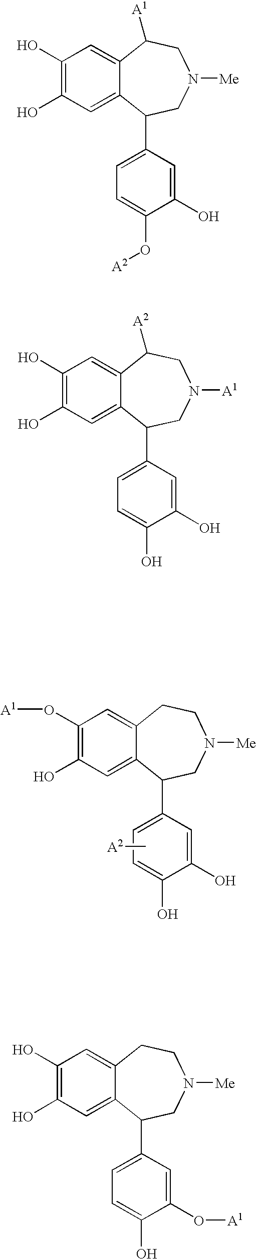
Group X

Catechol compounds IX include phosphonate analogs of styryl catechol compounds (Di Santo et al (2003) Pure and Applied Chemistry 75(2-3):195-206; Xu et al (2003) Bioorganic & Medicinal Chemistry 11(17):3589-3593); Lamidey et al (2002) Helv. Chim. Acta 85(8):2328-2334; Zouhiri et al (2000) J. Med. Chem. 43(8):1533-1540; Zouhiri et al (2001) Tetrahedron Letters 42(46):8189-8192; Ouali et al (2000) J. Med. Chem. 43(10)1949-1957; Mazumder et al (1997) J. Med. Chem. 40:3057-3063; Mekouar et al (1998) J. Med. Chem. 41:2846-2857; Pommier et al (2000) Antiviral Res. 47(3):139-148; Mazumder et al (1995) Biochemistry 34:15111-15122; Yoo et al (2003) Farmaco 58(12):1243-1250; Lee et al (2002) Archiv der Pharmazie (Weinheim, Germany), 335(6):277-282; WO 98/45269; WO 99/48371; WO 00/63152; WO 99/66942; WO 01/00199; WO 00/59867; WO 00/77013) including analogs of chicoric acid (Reinke et al (2002) Jour. Med. Chem. 45(17):3669-3683; Robinson et al (1996) Proc. Natl. Acad. Sci. USA 93:6326-6331; Robinson et al (1996) Mol. Pharmacol. 50:846-855) and lithospermic acid (Abd-Elazem et al (2002) Antiviral Research 55(1):91-106; WO 02/026726).

Phosphonate analogs of styryl catechol compounds generally have Formula X:

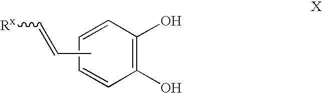

where R^{x} is a variety of scaffolds that is covalently attached to the catechol moiety through a single bond or a fused ring system.

Embodiments of Formula X compounds include: 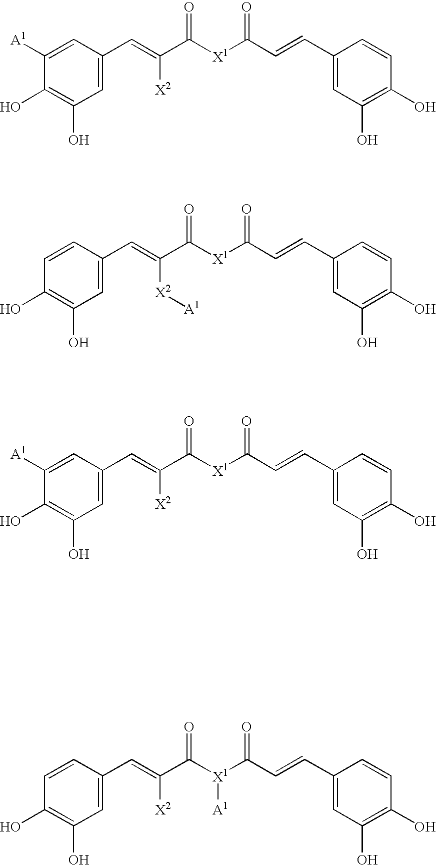
where X¹ is —NH(CH₂)nNH— where n is 1-6, alkylarylene, or arylene, and X² is CN, Br, or OH, and any carbon or hydroxyloxygen atom may be independently substituted with A².

Embodiments of Formula X compounds also include:

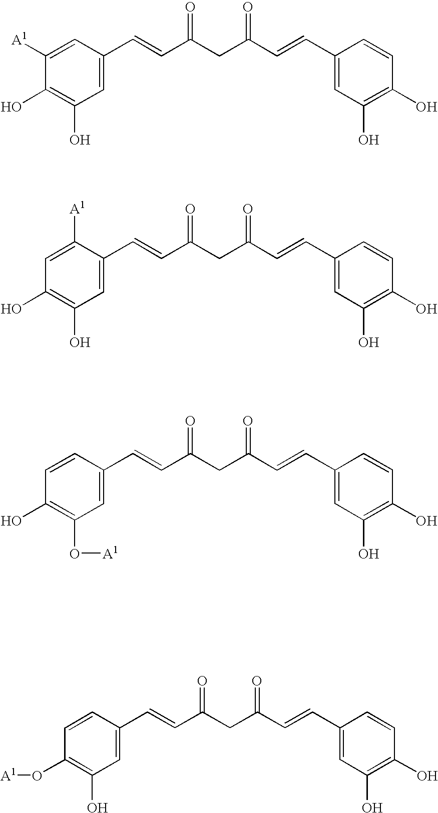

Embodiments of Formula X compounds also include:

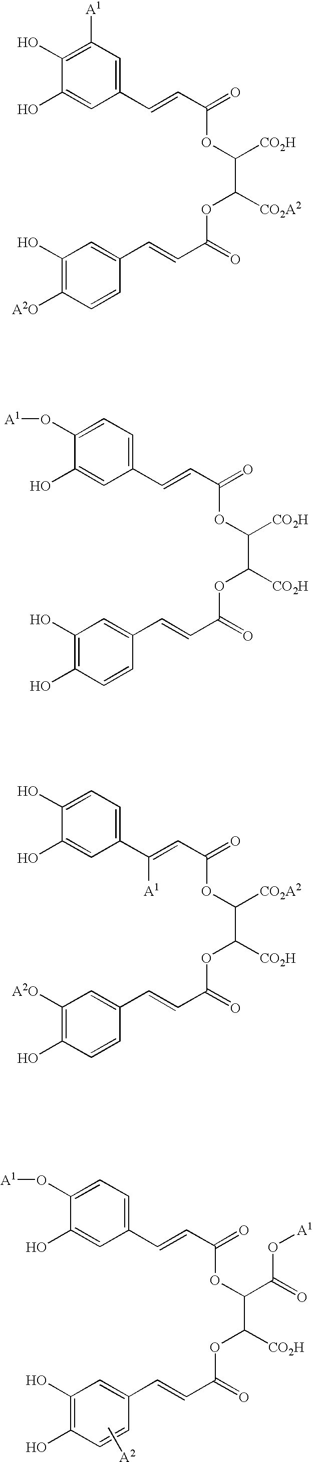

Embodiments of Formula X compounds also include:

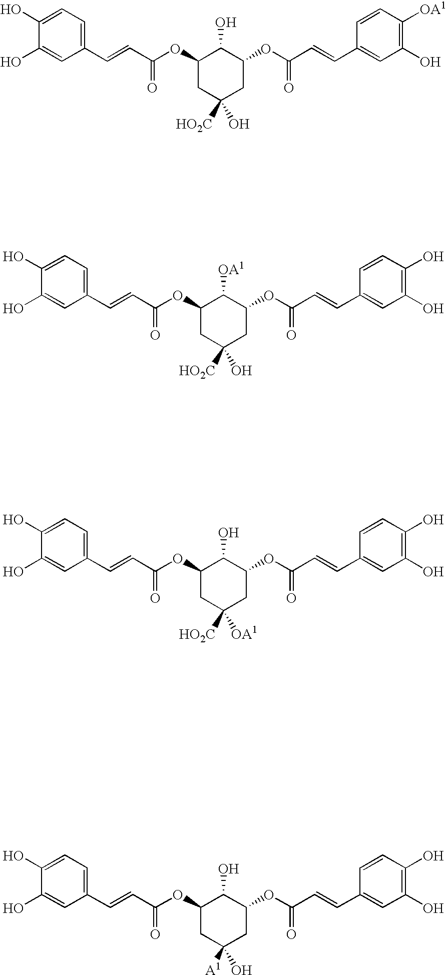

Embodiments of Formula X compounds also include: 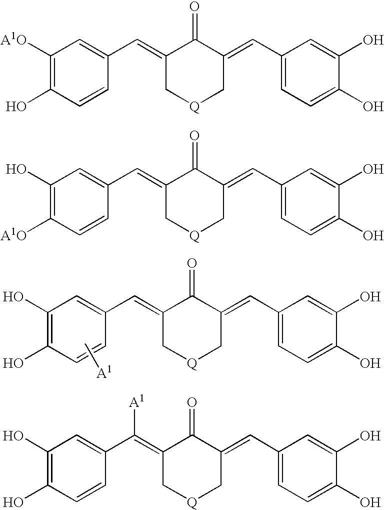
where Q is CH₂, O, S, NH, or NR.

Embodiments of Formula X compounds also include:

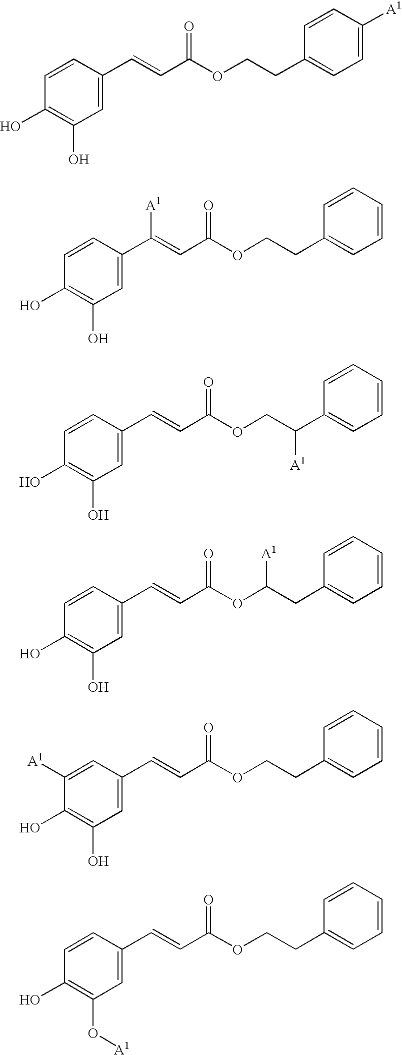

Embodiments of Formula X compounds also include:

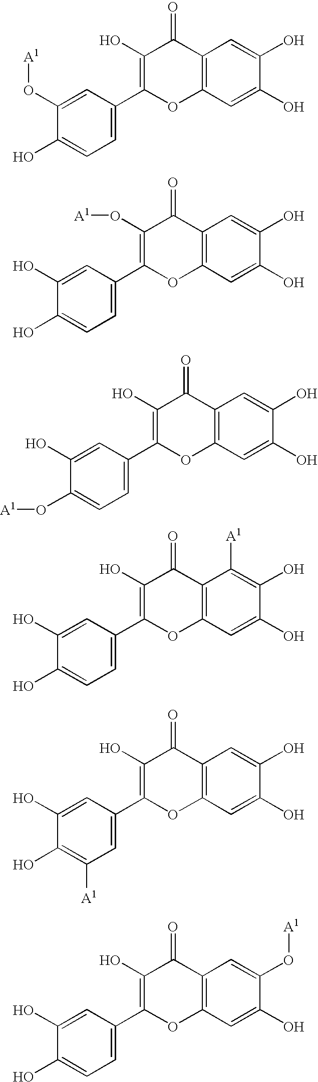

Embodiments of Formula X compounds also include:

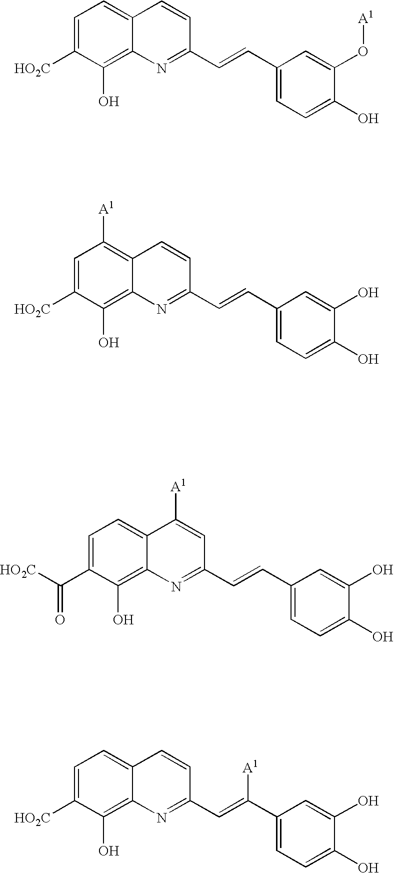

Embodiments of Formula X compounds also include:

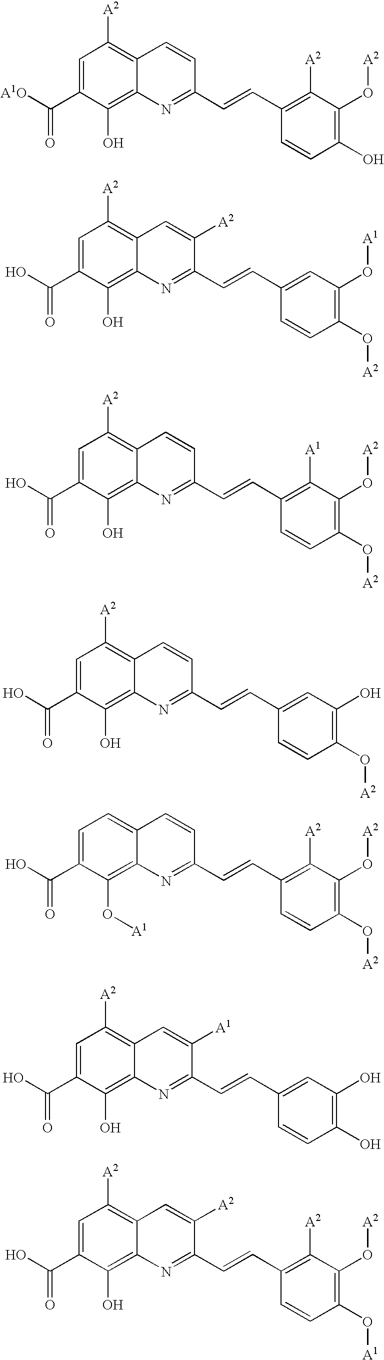

Embodiments of Formula X compounds also include:

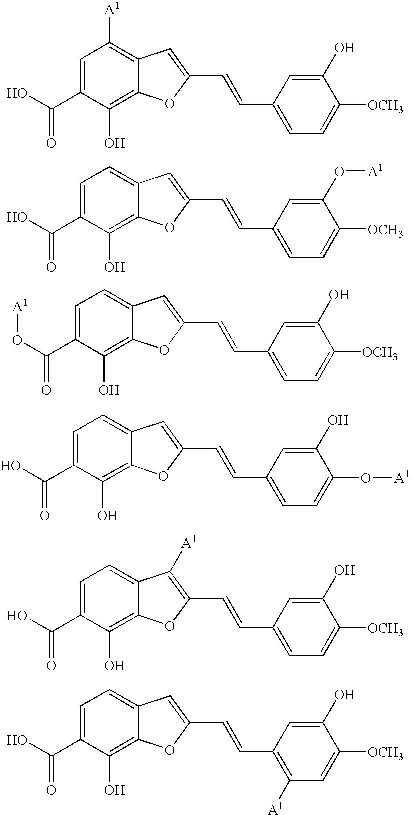

Embodiments of Formula X compounds also include: 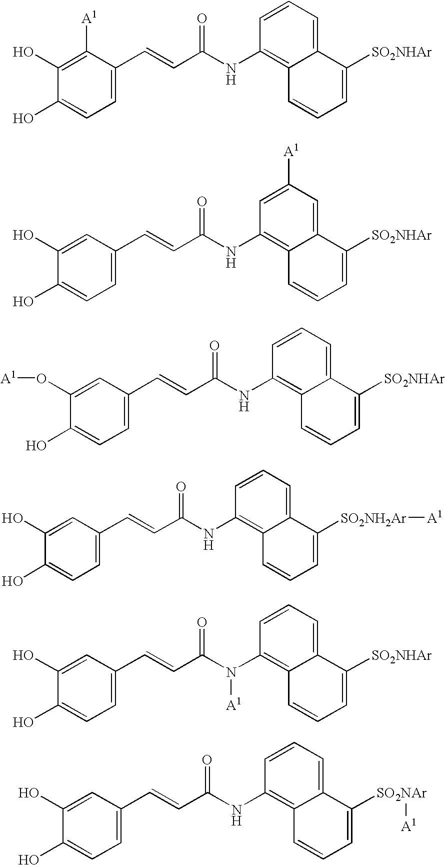
Group XI

In one aspect, the invention includes phosphonate analogs of benzimidazole compounds (WO 02/070491 A1) and bis-benzimidazole compounds (WO 95/08540; WO 95/19772; WO 98/38170; Pluymers et al (2000) Mol. Pharmacol. 58:641-648) having Formula XI:

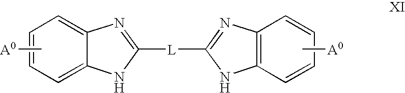

where Formula XI compounds may be further subsituted with fused ring systems, and L is a linker.

Embodiments of Formula XI compounds include:

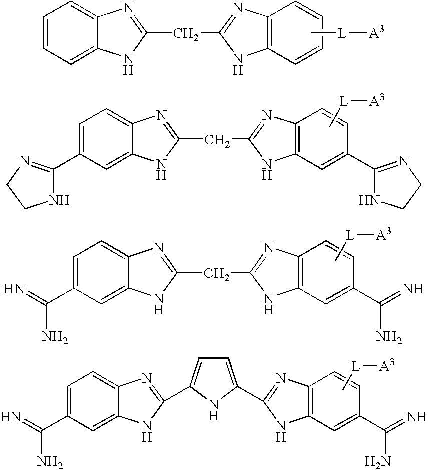

Further embodiments of Formula XI compounds include: 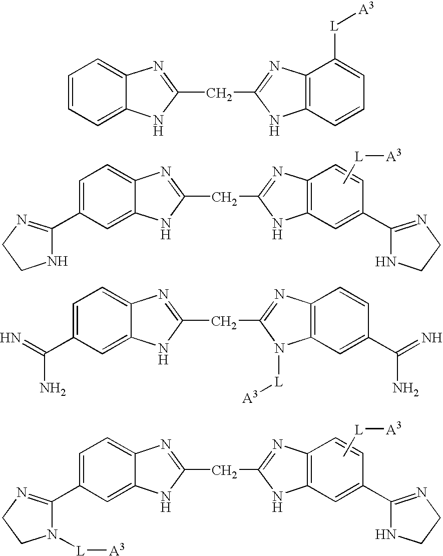
Group XII

In one aspect, the invention includes phosphonate analogs of indoloquinoxaline compounds (WO 96/00067) having Formula XII:

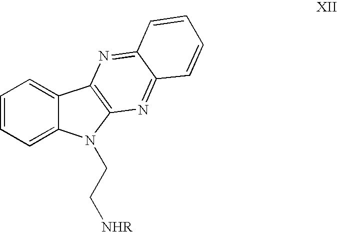

Embodiments of Formula XII compounds include: 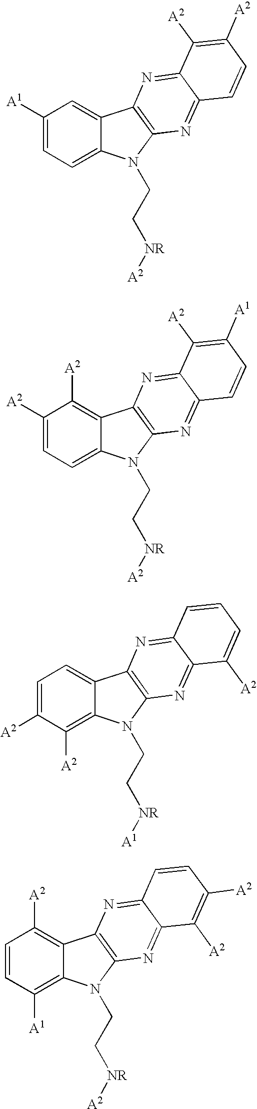
Group XIII

In one aspect, the invention includes phosphonate analogs of acridine compounds (Thale et al (2002) J. Org. Chem. 67:9384-9391) including phosphonate analogs of bis-acridine compounds (Turpin et al (1998) Antimicrob. Agents Chemother. 42:487-494; WO 97/38999) having Formula XIII:

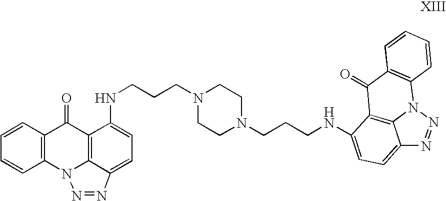

Embodiments of Formula XIII compounds include: 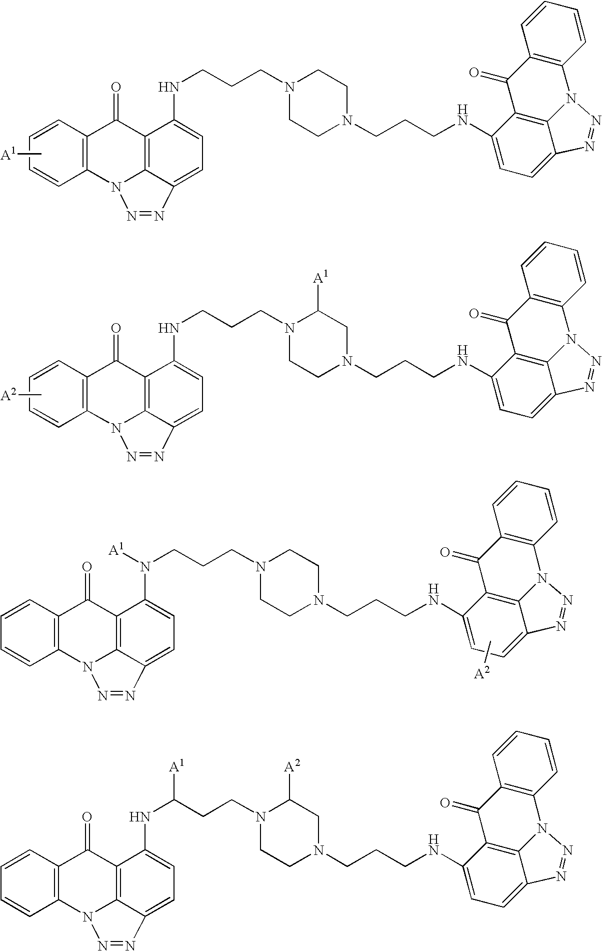
Group XIV

In one aspect, the invention includes phosphonate analogs of polyamide, DNA binding compounds (Fesen et al (1993) Proc. Natl. Acad. Sci. USA 90:2399-2403; Carteau et al (1993) Biochem. Biophys. Res. Commun. 192:1409-1414; Carteau et al (1994) Biochem. Pharmacol. 47:1821-1826; Mazumder et al (1995) AIDS Res. Hum. Retroviruses 11:115-125; Bouziane et al (1996) J. Biol. Chem. 271:10359-10364; Billich et al (1992) Antiviral Chem. Chemother. 3:113-119; Ryabinin et al (2000) Eur. J. Med. Chem. 35(11):989-1000), such as polypyrrole amide phosphonate oligomers (Neamati et al (1998) Mol. Pharmacol. 54:280-290; Wang et al (1992) J. Med. Chem. 35:2890-2897) having Formula XIV: 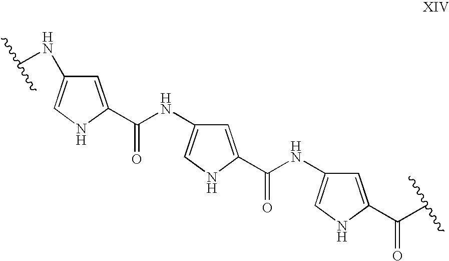
where the wavy lines 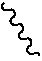
indicate the depicted structure is a substructure of a repeating polymer molecule.

Embodiments of Formula XIV compounds include: 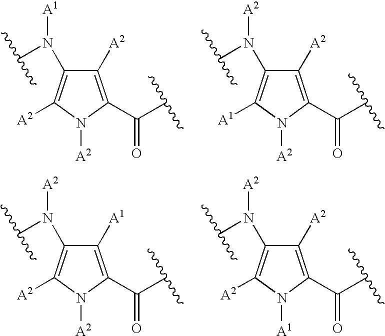
where one or more of the pyrrole amide monomer units in the polypyrrole amide molecule are substituted at one or more locations with a phosphonate group.
Group XV

In one aspect, the invention includes phosphonate analogs of [6,6] bicyclic compounds (Hazuda et al (1999) Antiviral. Chem. Chemother. 10:63; U.S. Pat. No. 6,541,515; Singh et al (1998) Tetrahedron Lett. 39:2243-2246; GB 2306476; U.S. Pat. No. 5,759,842), including integramycins (Singh et al (2002) Organic Letters 4(7):1123-1126) and fungal metabolites having Formula XV:

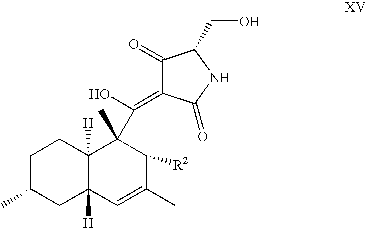

Embodiments of Formula XV compounds include:

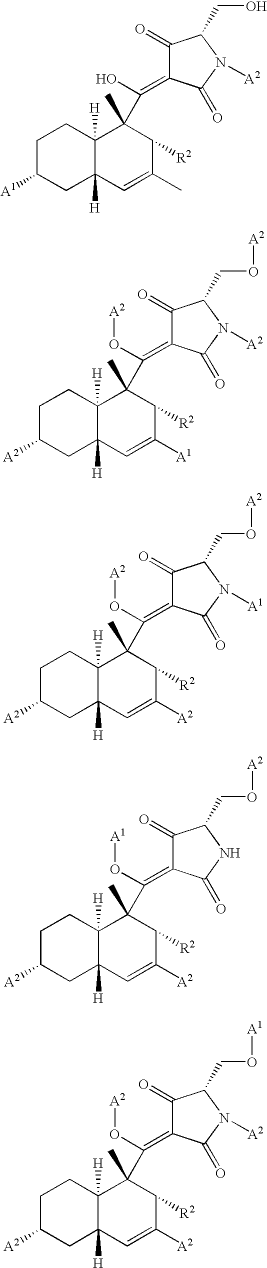

Embodiments of integramycin phosphonate Formula XV compounds also include:

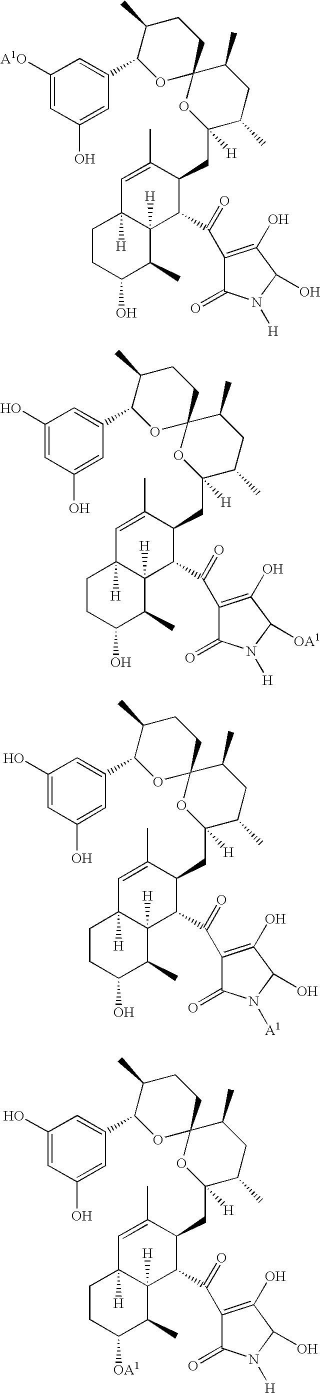

Embodiments of Formula XV compounds include phosphonate equicetin compounds having the structures: 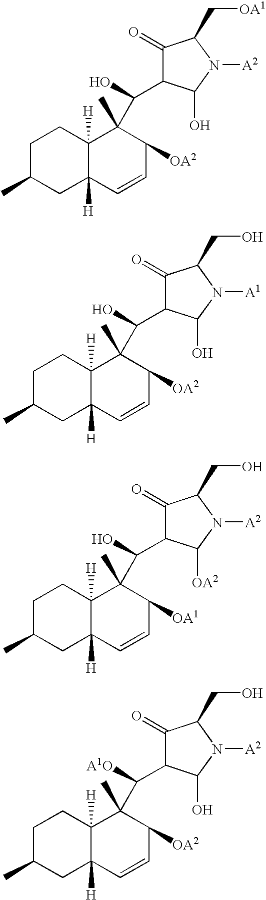
Group XVI

In one aspect, the invention includes phosphonate analogs of [6,6] bicyclic terpenoid compounds (GB 2319026) having Formula XVI:

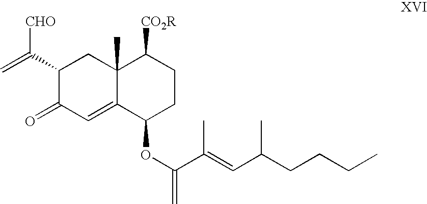

Embodiments of Formula XVI compounds include phosphonate [6,6] bicyclic terpenoid compounds having the structures: 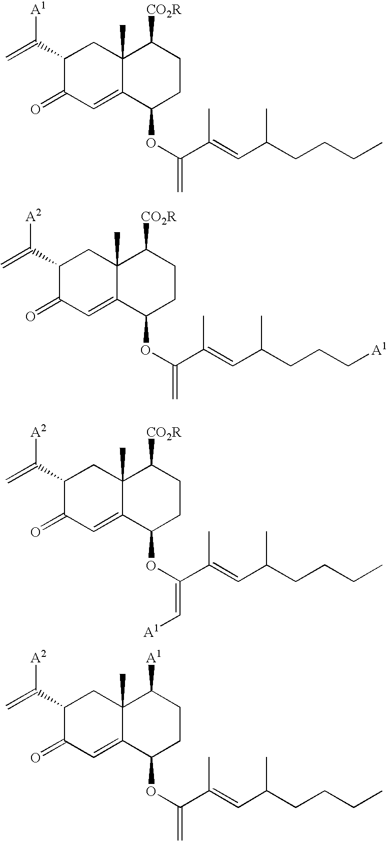
Group XVII

In one aspect, the invention includes phosphonate analogs of aurintricarboxylic acid compounds (Cushman et al (1992) Biochem. Biophys. Res. Commun. 185:85-90; Cushman et al (1995) J. Med. Chem. 38:443-452; Cushman et al (1991) J. Med. Chem. 34(1):337-342) having Formula XVII:

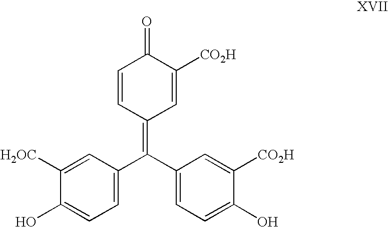

Embodiments of Formula XVII compounds include phosphonate aurintricarboxylic acid compounds having the structures: 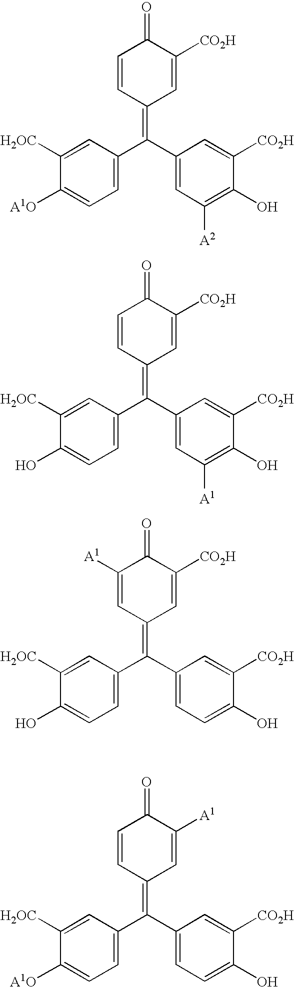
Group XVIII

In one aspect, the invention includes phosphonate analogs of integrastatin compounds (Foot et al (2003) Organic Letters 5(23):4441-444; Singh et al (2002) Tetrahedron Lett. 43:2351-2354; WO 01/09114) having Formula XVIII:

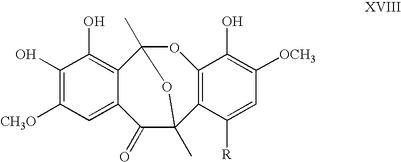

Embodiments of Formula XVIII compounds include phosphonate integrastatin compounds having the structures: 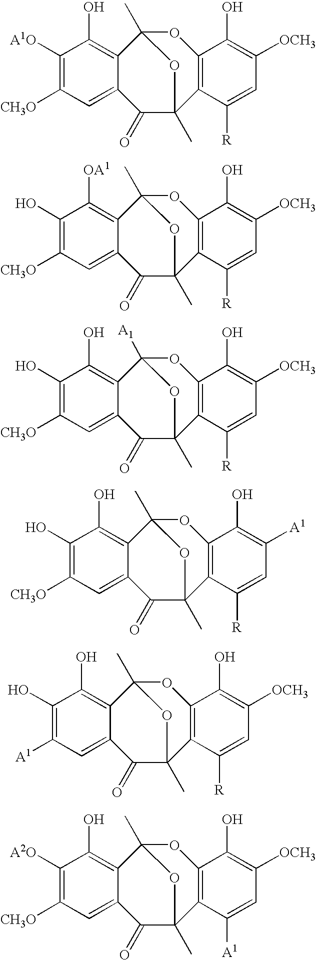
Group XIX

In one aspect, the invention includes phosphonate analogs of 6-(arylazo)pyridoxal-5-phosphate compounds (WO 03/082881 A2) having Formula XIX:

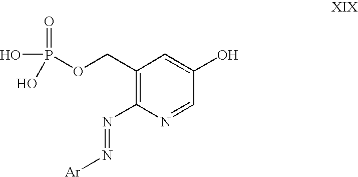

Embodiments of Formula XIX compounds include phosphonate 6-(arylazo)pyridoxal-5-phosphate compounds having the structures: 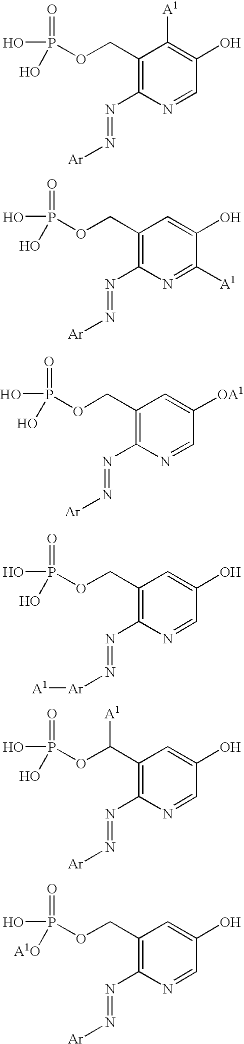
Group XX

In one aspect, the invention includes phosphonate analogs of 1,3-oxazine-, 1,3-thiazine-, pyran-, 1,4-oxazepine-, and 1,4-thiazepine-fused naphthalene compounds (WO 03/024941 A1) having Formula XX structures.

R¹ is H, (un)substituted C1-6 alkyl, halo, NO₂, NH₂, CO₂H, (un)substituted aryl, optionally benzene-fused 5- or 6-membered aromatic or saturated. heterocyclyl containing 1-3 heteroatoms selected from N, S, and O, (un)substituted aryl-carbonylamino;

R² and R³ are independently H, C₁₋₆ alkyl or alkoxy, halo, NH₂, C₁₋₆ alkylamino, di(C1-6 alkyl)amino, NO₂, CN, CONH₂, CO₂H, C₂₋₇ alkylcarbonylamino, C₃₋₁₃ alkoxycarbonylaminoalkoxy, C₁₋₆ aminoalkoxy, C₃₋₁₃ alkylcarbonylaminoalkoxy;

X=O, S;

Y=CR⁴R⁵, CR⁴R⁵CH₂, CH₂CR⁴R⁵ wherein R⁴, R⁵=H, C1-6 alkyl, CO₂H, C₂₋₆ alkoxycarbonyl, optionally substituted aryl, C₂₋₇ alkoxycarbonylalkyl, hydroxyalkyl, C₃₋₇ cycloalkyl-alkyl, or arylalkyl; and

Z=CH₂, (un)substituted NH.

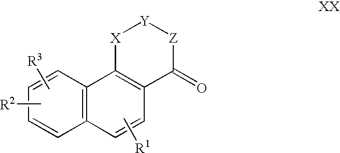

Embodiments of Formula XX compounds include phosphonate 1,3-oxazine-, 1,3-thiazine-, pyran-, 1,4-oxazepine-, and 1,4-thiazepine-fused naphthalene compounds having the structures: 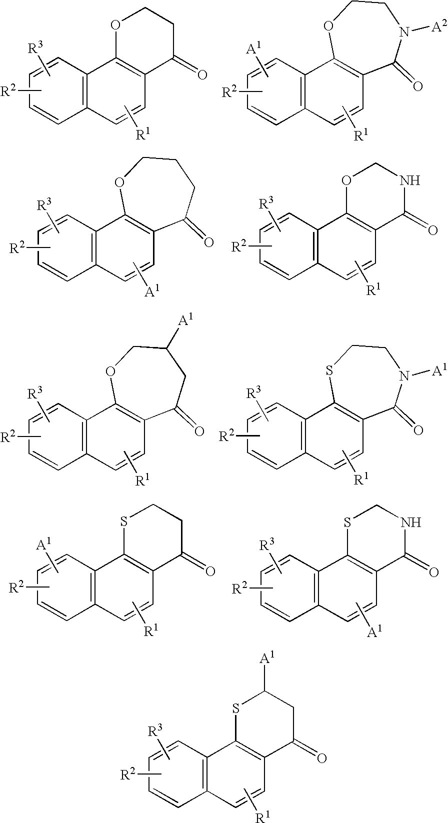
Group XXI

In one aspect, the invention includes phosphonate analogs of chaetochromin compounds derived from chaetochromin fermentation products and their chemically modified derivatives (WO 98/34932) including naphtho-γ-pyrones (Singh et al (2003) Bioorganic & Med. Chemistry Letters 13(4):713-717 having Formula XXI.

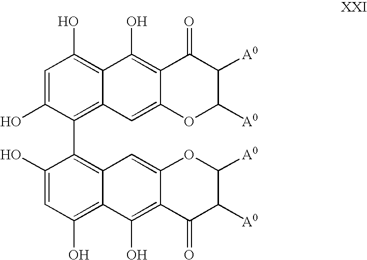

Formula XXI compounds further include phosphonate unsaturated (isochaetochromin D₁) and further oxidized lactone (oxychaeotochromin B) analogs of isochaetochromin B₁ and B₂ according to following structures:

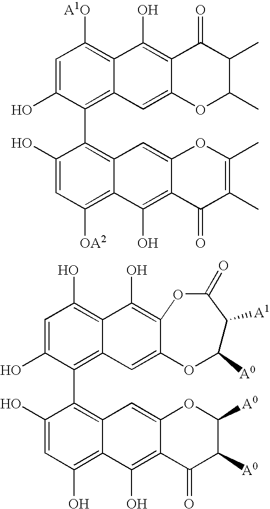

Embodiments of phosphonate analogs of chaetochromin compounds also include the structures:

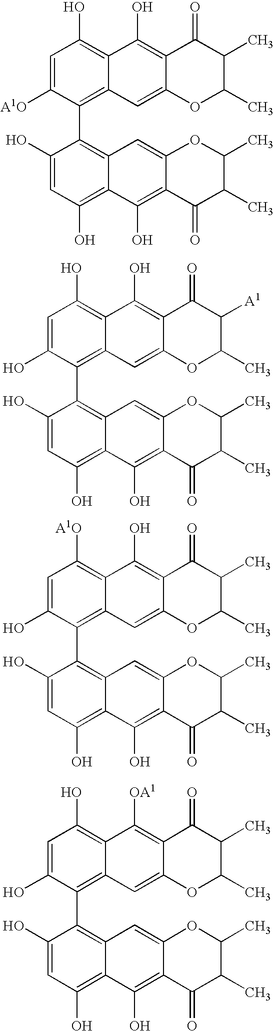

The invention includes all rotational isomers, i.e. atropisomers, which may exist as stable enantiomers due to slow rotation around the single bond connecting the aryl rings of Formula XXI compounds.

Group XXII

In one aspect, the invention includes phosphonate analogs of hydroxyphenylundecane compounds derived from fermentation products and their chemically modified derivatives (GB 2327674) including integracins (Singh et al (2002) Tetrahedron Lett. 43(9):1617-1620) having Formula XXII structures:

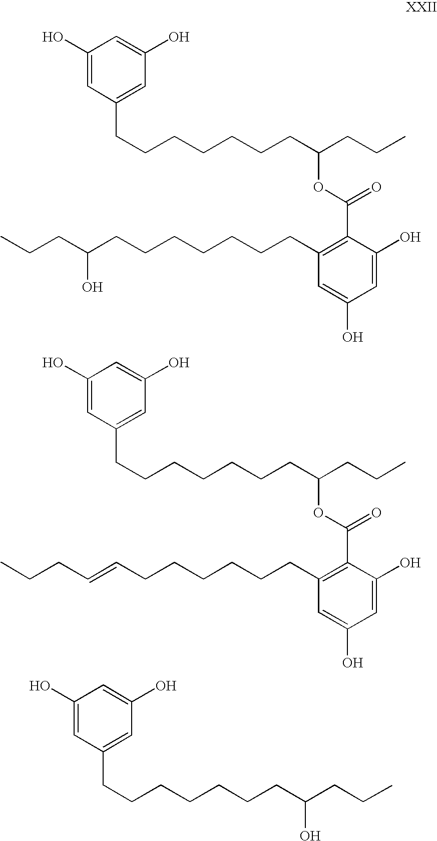

Embodiments of hydroxyphenylundecane phosphonate compounds Formula XXII include the structures:

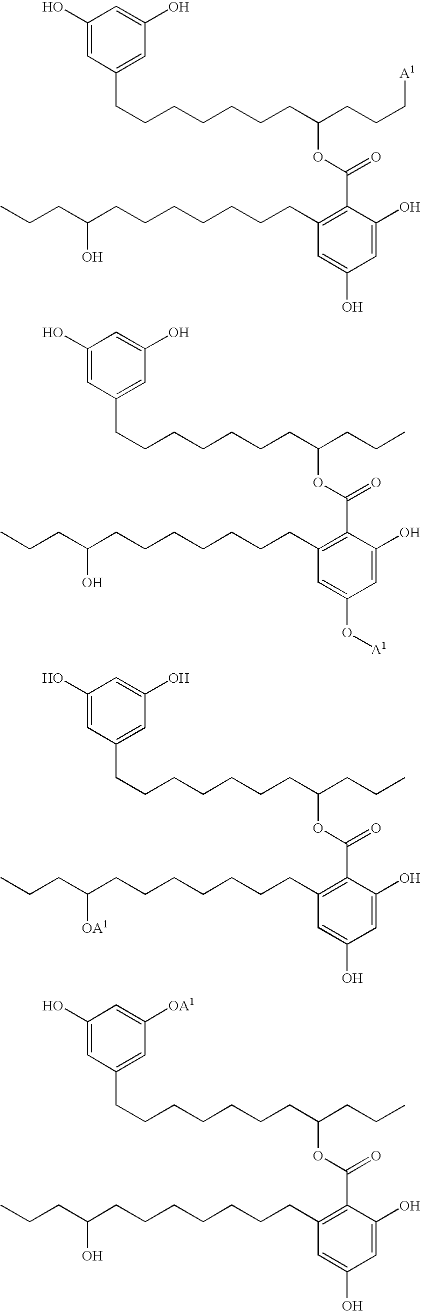

Embodiments of hydroxyphenylundecane phosphonate compounds Formula XXII also include the structures:

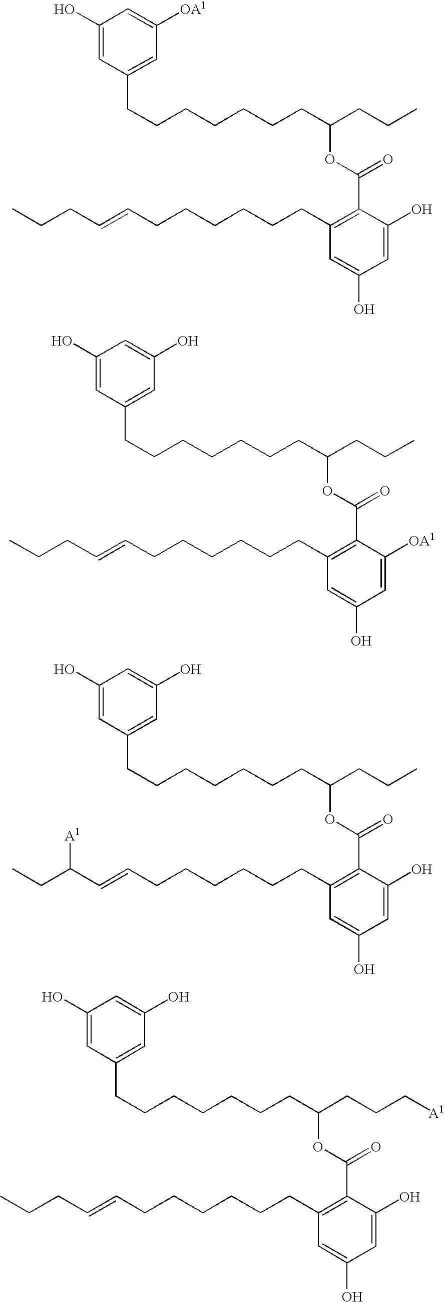

Embodiments of hydroxyphenylundecane phosphonate compounds Formula XXII also include the structures: 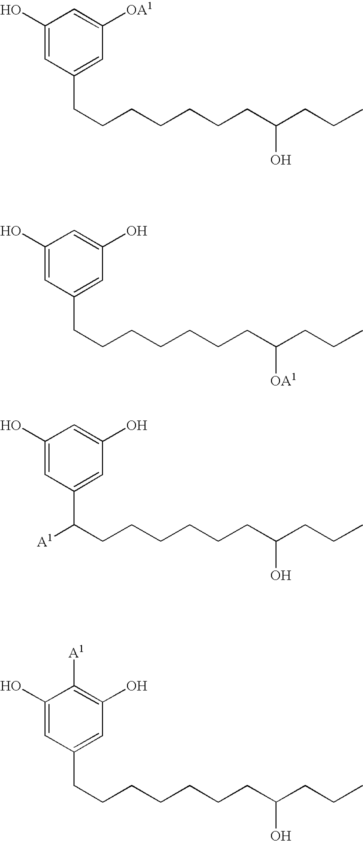
Group XXIII

In one aspect, the invention includes phosphonate analogs of: (i) tetracyclic steroidal compounds derived from fermentation products and their chemically modified derivatives (Singh et al (2003) Jour. of Natural Products 66(10):1338-1344; WO 00/36132); and (ii) tetracyclic triterpenoid compounds, such as integracides (Singh et al (2003) Bioorganic & Med. Chemistry 11(7):1577-1582).

Embodiments of phosphonate integracide Formula XXIII compounds include the structure: 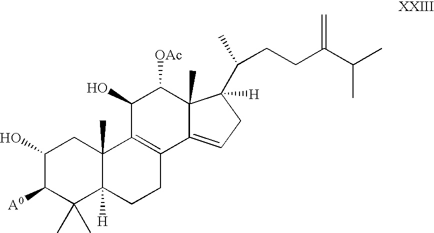
where at least one carbon or oxygen atom is substituted with an A¹ group, and any aryl or sultam ring carbon atom may be substituted with an A² group, including the exemplary structures:

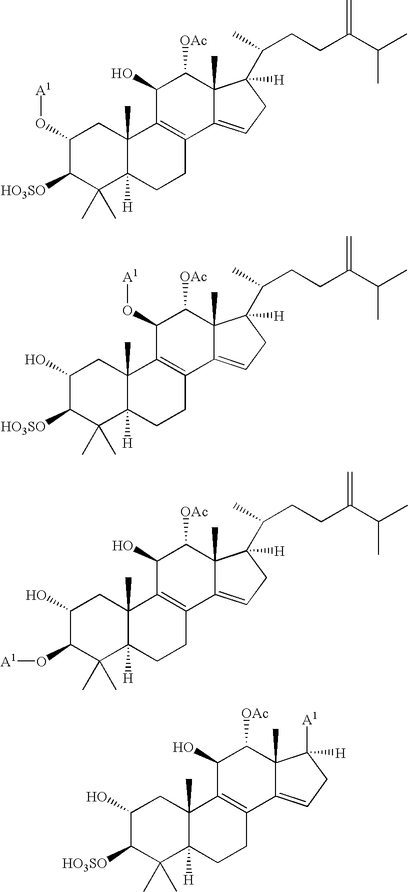

Embodiments of phosphonate integracide B Formula XXIII compounds also include the structures: 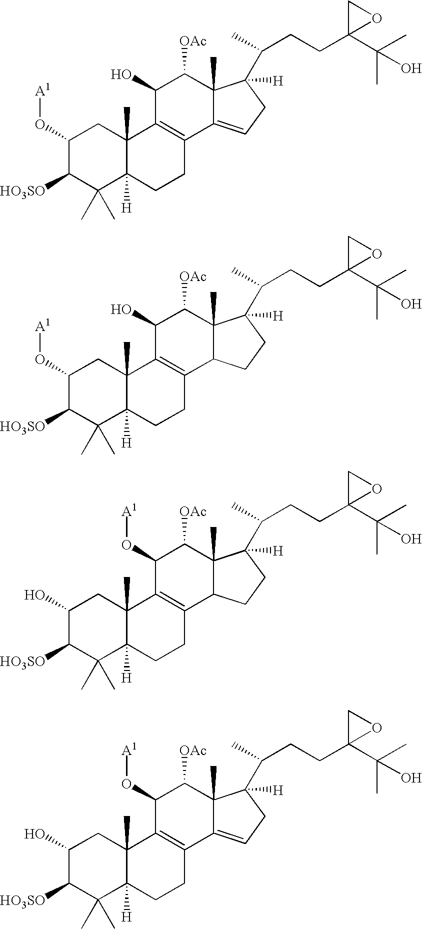
Group XXIV

In one aspect, the invention includes phosphonate analogs of plant natural products including: (i) glycerrhenitic and betulonic acids (Semenova et al (2003) Doklady Biochemistry and Biophysics 391:218-220); (ii) compounds from Coleus parvifolius Benth. (Tewtrakul et al (2003) Phytotherapy Research 17(3):232-239); (iii) eudesmane-type sesquiterpenes and aporphine alkaloid lindechunines from Lindera chunii roots including hemandonine, laurolistine, 7-oxohemangerine and lindechunine A (Zhang et al (2002) Chemical & Pharmaceutical Bulletin 50(9):1195-1200); and (iv) lithospermic acid (Abd-Elazem et al (2002) Antiviral Research 55(1):91-106; WO 02/026726).

Embodiments of Formula XXIV glycerrhenitic and betulonic acid phosphonate compounds include the structures:

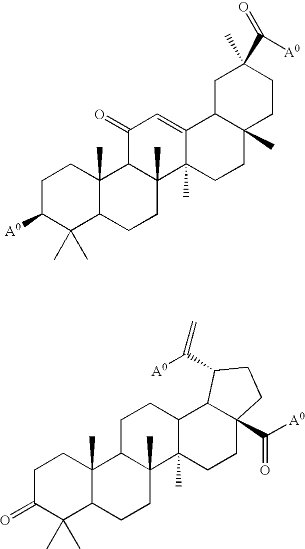

Embodiments of Formula XXIV compounds also include the structures:

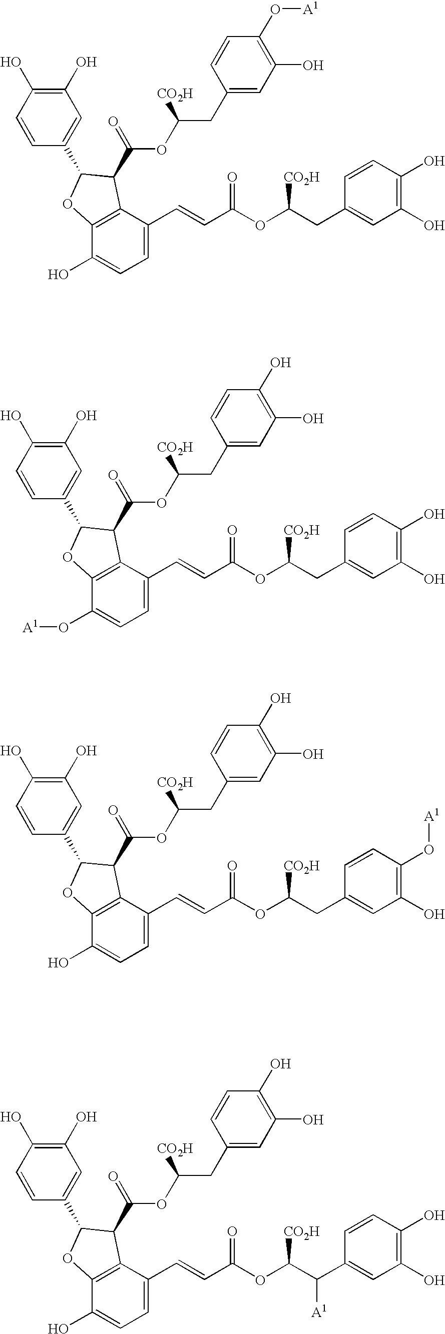

Embodiments of laurolistine phosphonate Formula XXIV compounds include the structures: 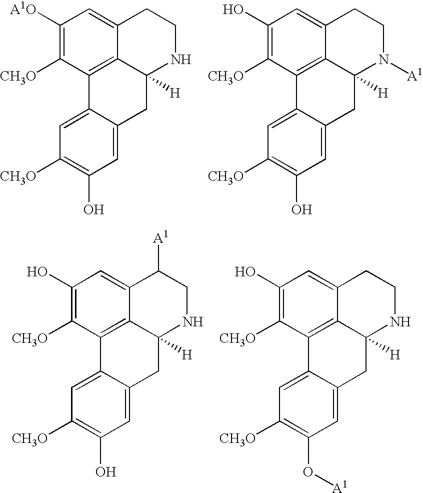
Group XXV

In one aspect, the invention includes phosphonate analogs of spiro ketal compounds derived from fungal cultures and fungus, and their chemically modified derivatives (Neamati, N. (2002) Expert Opinion Therapeutic Patents 12(5):709-724, compound 47, Table 2, p. 714) with the Formula XXV structure:

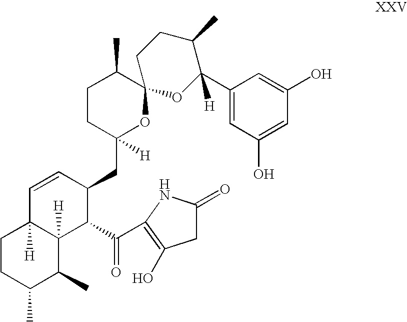

Embodiments of spiro ketal phosphonate Formula XXV compounds include the structures: 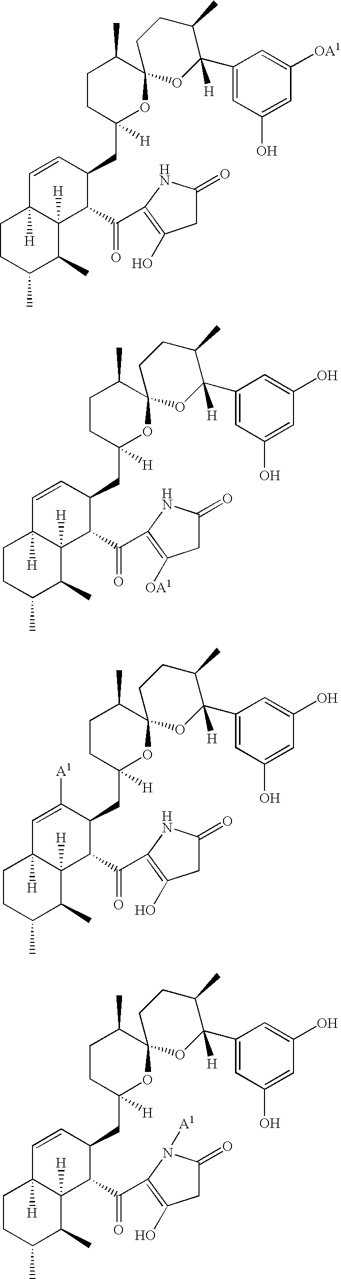
Group XXVI

In one aspect, the invention includes phosphonate analogs of aromatic lactone compounds derived from lichen extracts, and their chemically modified derivatives (Neamati et al (1997) J. Med. Chem. 40:942-951; Neamati et al (1997) Antimicrob. Agents Chemother. 41:385-393). Phosphonate aromatic lactone Formula XXVI compounds include the structures:

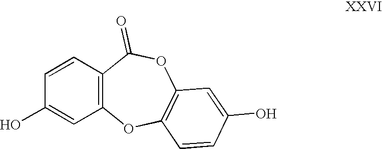

Embodiments of phosphonate aromatic lactone Formula XXVI compounds include the structures: 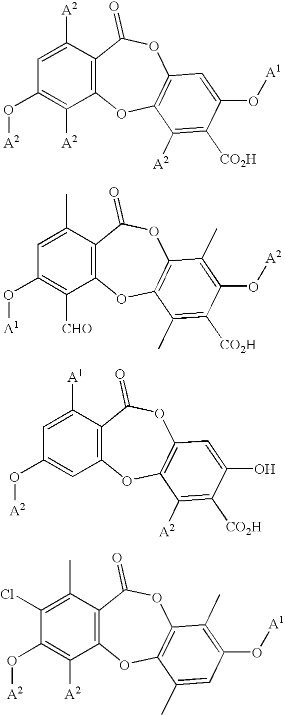
Group XXVII

In one aspect, the invention includes phosphonate analogs of salicylhydrazide and mercaptosalicylhydrazide compounds (Neamati et al (2002) J. Med. Chem. 45(26): 5661-5670; Neamati et al (1998) J. Med. Chem. 41:3202-3209; Zhao et al (1997) J. Med. Chem. 40:937-941; WO 00/53577) which have Formula XXVII structures:

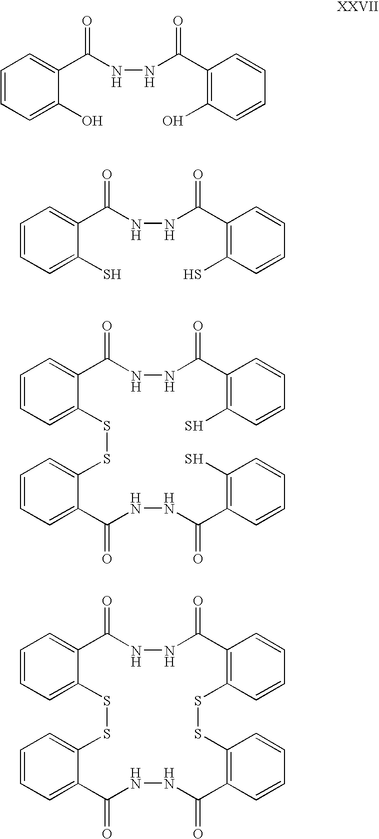

where each of the phenyl rings, N, S, or hydroxyloxygen atoms in the structures above may be independently substituted with A⁰ groups.

Embodiments of Formula XXVII compounds include the structures:

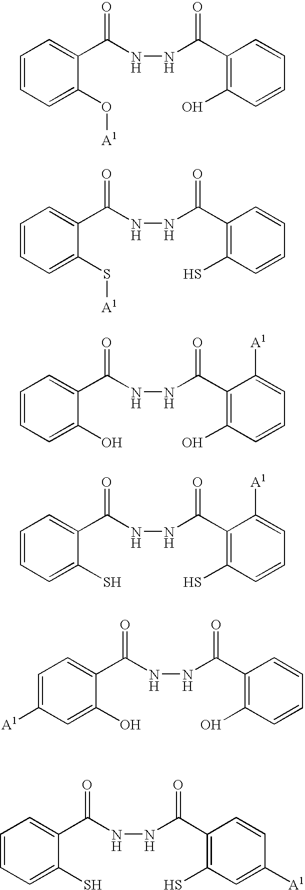

Embodiments of Formula XXVII compounds also include the structures: 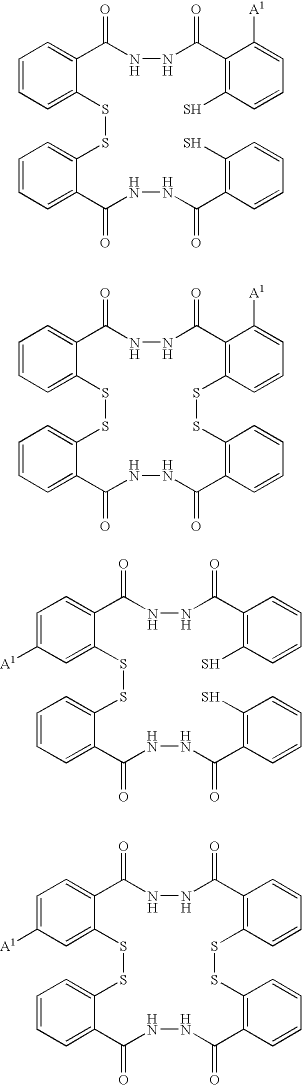
Group XXVIII

In one aspect, the invention includes phosphonate analogs of thiazolothiazepine compounds (Neamati et al (1999) J. Med. Chem. 42:3334-3341; WO 00/68235).

Embodiments of Formula XXVIII thiazolothiazepine phosphonate compounds include the structures:

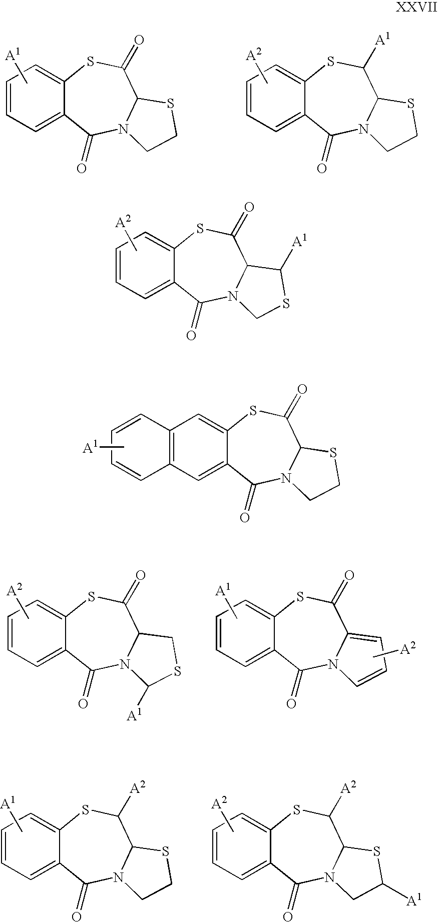

Embodiments of Formula XXVIII thiazolothiazepine phosphonate compounds also include the structures: 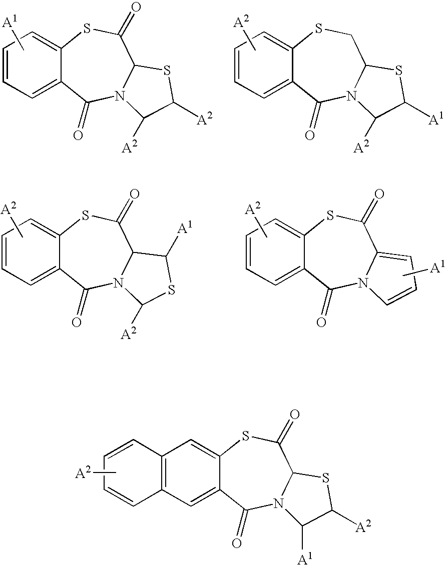
Group XXIX

In one aspect, the invention includes phosphonate analogs of benzodiazepine hydrazide compounds (WO 98/18473). Embodiments of Formula XXIX benzodiazepine hydrazide phosphonate compounds include the structures: 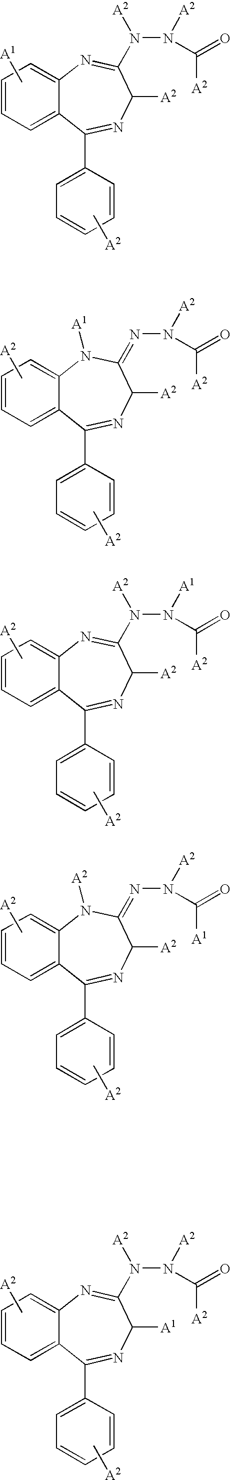 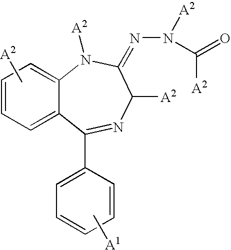
Group XXX

In one aspect, the invention includes phosphonate analogs of coumarin compounds (Mao et al (2002) Chemical & Pharmaceutical Bulletin 50(12):1634-1637; Chavda et al (2002) Indian Journal of Chemistry, Section B: Organic Chemistry Including Medicinal Chemistry 41B(10):2197-2199; Zhao et al (1997) J. Med. Chem. 40:242-249; Mazumder et al (1996) J. Med. Chem. 39:2472-2481; Hong et al (1997) J. Med. Chem. 40-930-936; JP 12178267). Coumarin phosphonate compounds include Lamellarin-type marine natural products (Reddy et al (1999) J. Med. Chem. 42(11):1901-1907; Zhang et al (2003) Abstracts of Papers, 226th ACS National Meeting, New York, N.Y., United States, Sep. 7-11, 2003; Ridley et al (2002) Bioorganic & Medicinal Chemistry 10(10)3285-3290; Handy et al Abstracts of Papers, 224th ACS National Meeting, Boston, Mass., Aug. 18-22, 2002).

Exemplary phosphonate coumarin Formula XXX compounds include the structures: 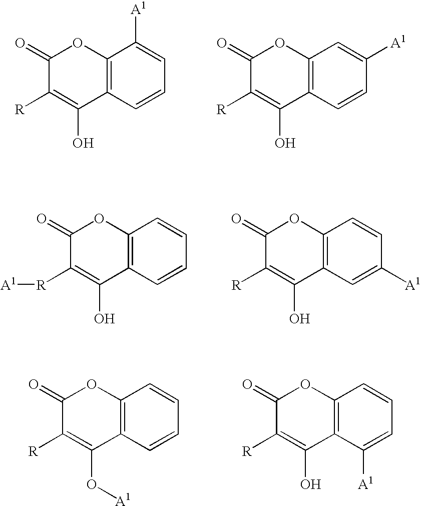
where R is H, C₁-C₈ alkyl, C₁-C₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heteroaryl, or C₂-C₂₀ substituted heteroaryl.

Exemplary phosphonate coumarin dimer Formula XXX compounds include the structures: 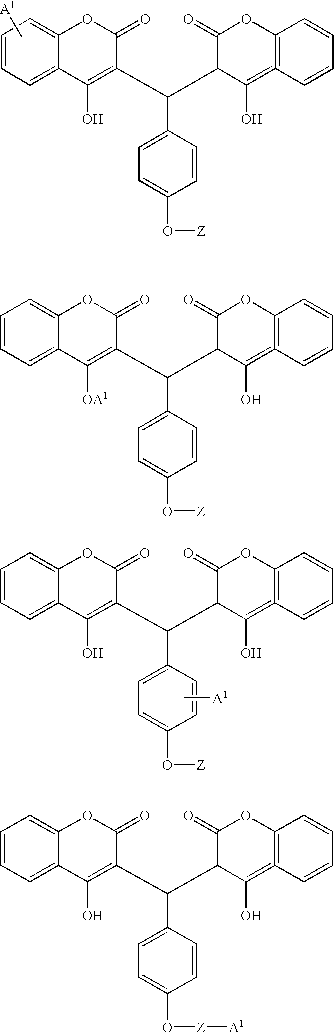
where Z is —C(O)Ar or —SO₂R.

Exemplary phosphonate Lamellarin Formula XXX compounds include the structures: 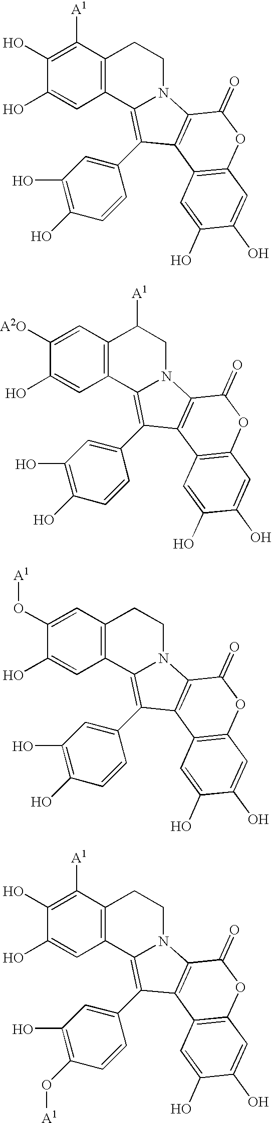 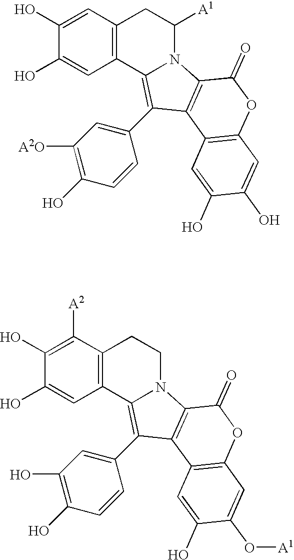
Group XXXI

In one aspect, the invention includes phosphonate analogs of brominated polyacetylene marine natural products from sponges such as Diplastrella sp. (Lerch et al (2003) Journal of Natural Products 66(5):667-670). Brominated polyacetylene phosphonate Formula XXXI compounds, including sulfated and sufonated analogs, have the structure:

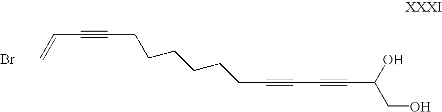

Exemplary phosphonate brominated polyacetylene Formula XXXI compounds include the structures: 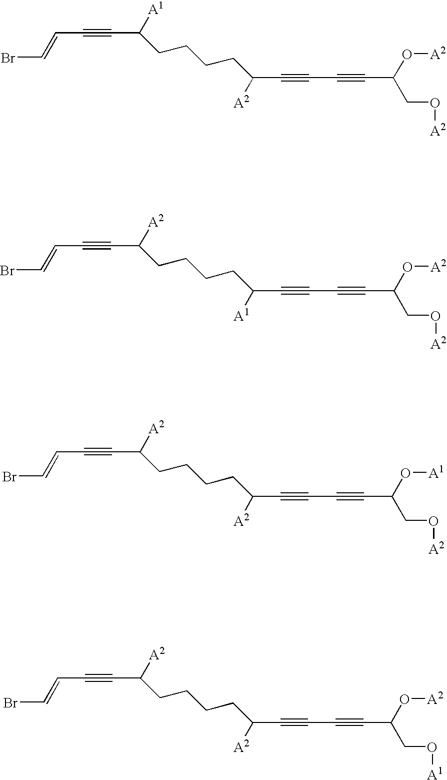
Group XXXII

In one aspect, the invention includes phosphonate analogs of cobalamin (Vitamin B12) compounds (Weinberg et al (1998) Biochem. Biophys. Res. Commun. 246:393-397) including structure XXXII.

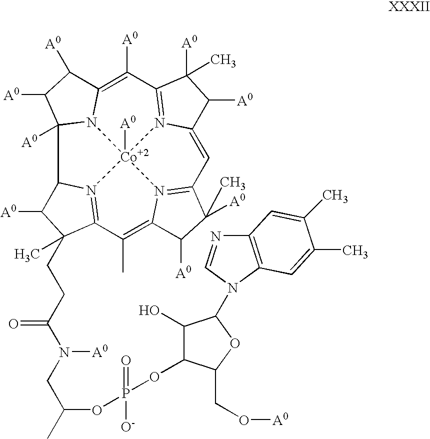

Exemplary phosphonate cobalamin Formula XXXI compounds include the structures: 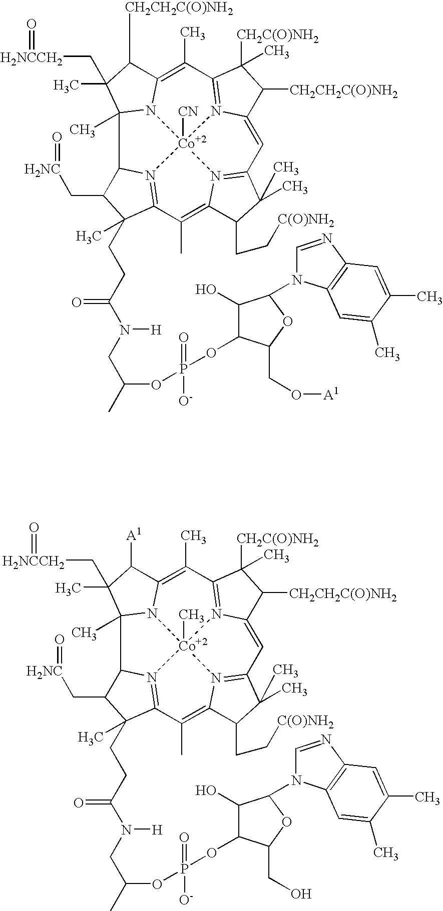
and all phosphonate analogs of cobalt complexes of corrin, cobyrinic acid and corrole ring systems (Merck Index, Eleventh Edition (1989), entry 9921).
Group XXXIII

In one aspect, the invention includes phosphonate analogs of hydroxylated aromatic compounds (Burke et al (1995) J. Med. Chem. 38:4171-4178), including: tetracycline compounds (Neamati et al (1997) Mol. Pharmacol. 52:1041-1055); anthraquinones and naphthoquinones (Fesen et al (1993) Proc. Natl. Acad. Sci. USA 90:2399-2403; Farnet et al (1996) Proc. Nat. Acad. Sci. USA 93:9742-9747); and flavones, flavanones, flavanols, and flavanoids (Lee et al (2003) Hakhak Hoechi (Korea) 47(1):46-51; Mateeva et al (2002) Jour. of Heterocyclic Chemistry 39(6):1251-1258; Li et al (2002) Tetrahedron Lett. 43(51):9467-9470; Tewtrakul et al (2992) Chemical & Pharmaceutical Bulletin 50(5):630-635; Fesen et al (1994) Biochem. Pharmacol. 48:595-608; Ahn et al (2002) Planta Medica 68(5):457-459; Lee et al (2003) Planta Medica 69(9):859-861) including thalassiolins (Rowley et al (2002) Bioorganic & Medicinal Chemistry 10(11):3619-3625); baicalein and baicalin (Ahn ety al (2001) Molecules and Cells 12(1):127-130); and benzopyrano-oxopyrimidotetrahydrothiazines (WO 02/051419 A2).

Exemplary embodiments of Formula XXXIII tetracycline phosphonate compounds include:

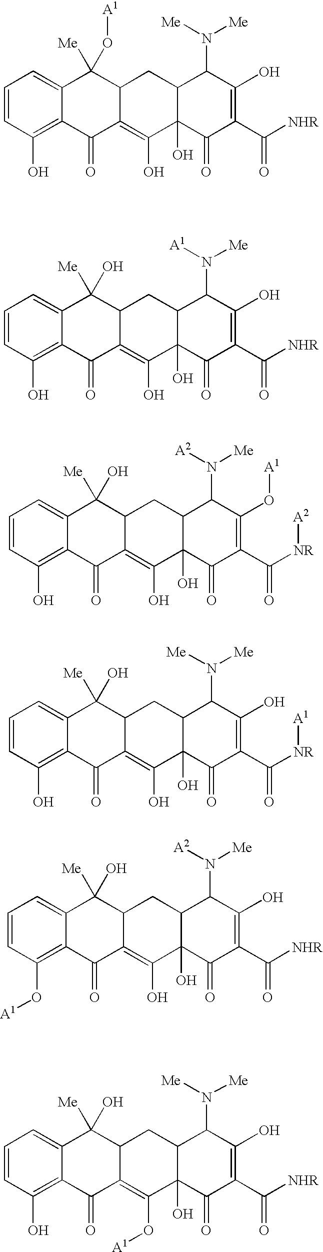

Exemplary embodiments of Formula XXXIII tetracycline phosphonate compounds further include:

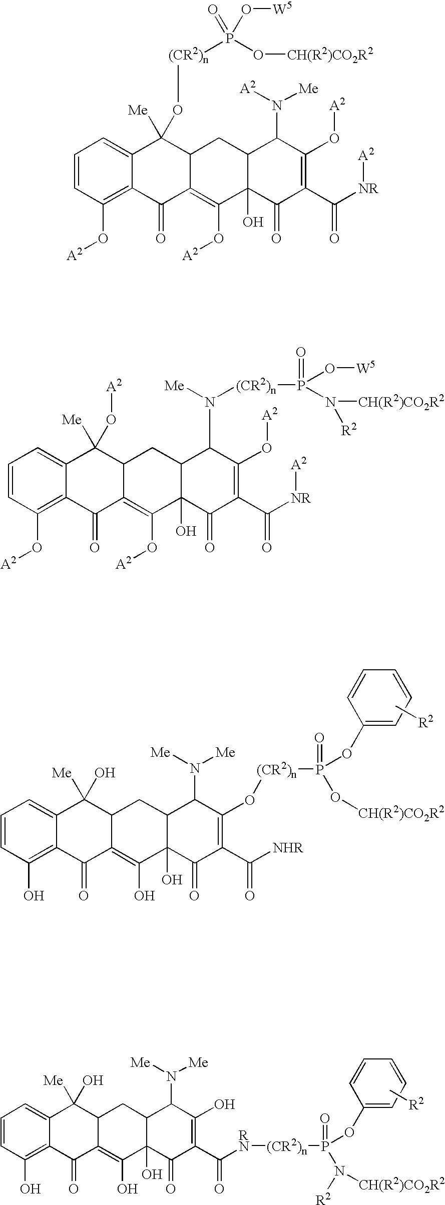

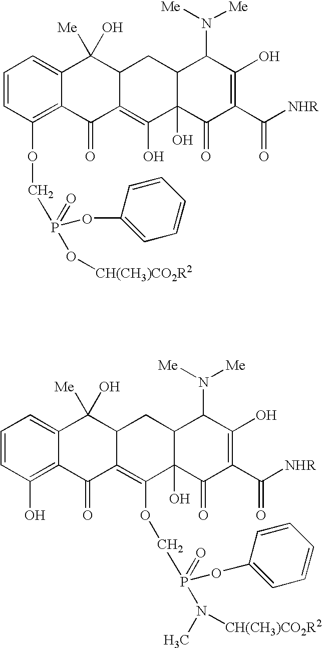

Exemplary embodiments of Formula XXXIII flavanol phosphonate compounds include phosphonate analogs of quercetin 3-O-(2″-galloyl)-α-L-arabinopyranoside such as the structures: 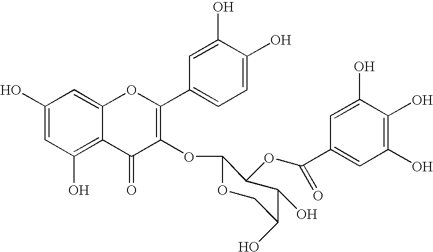
where at least one carbon or hydroxyloxygen atom is substituted with an A¹ group, and any carbon or hydroxyloxygen atom may be substituted with an A² group, including the exemplary structures:

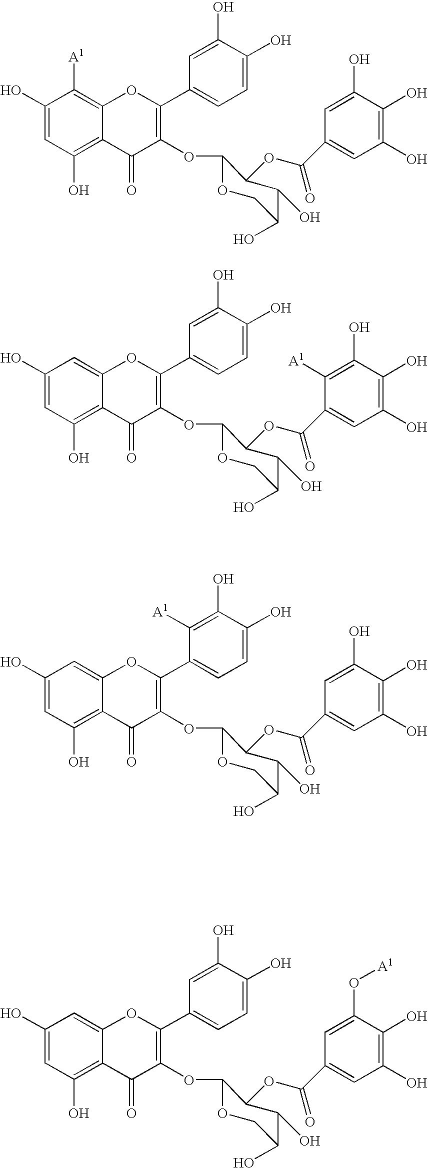

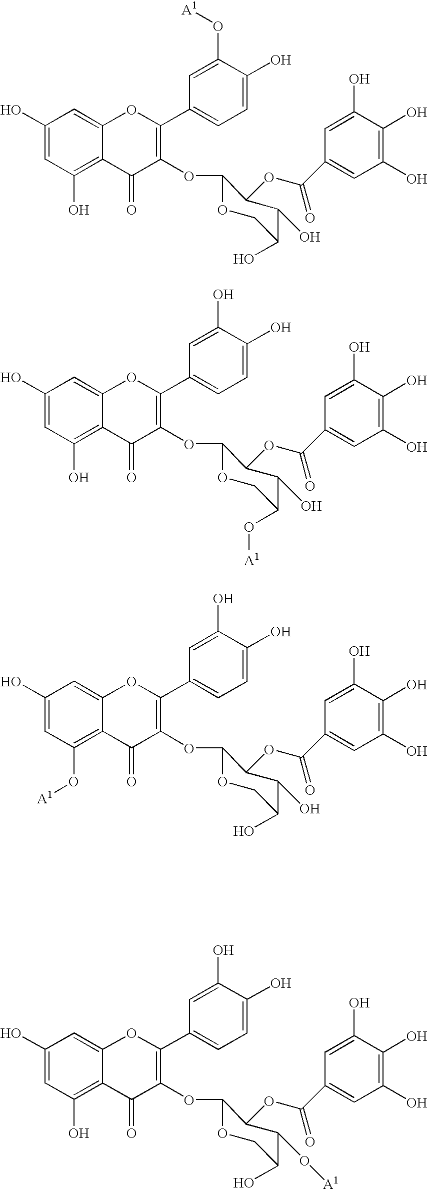

Exemplary Disaccharide catechol phosphonate Formula XXXIII compounds include the structures:

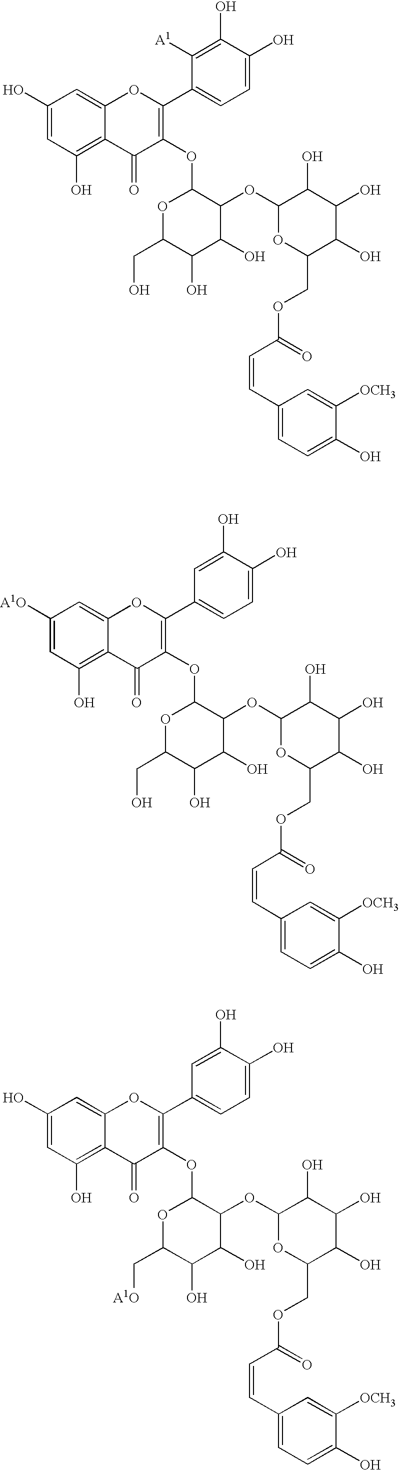

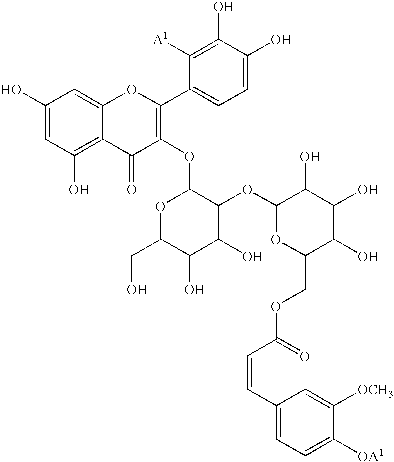

Exemplary flavonoid glucuronide phosphonate Formula XXXIII compounds include the structures: 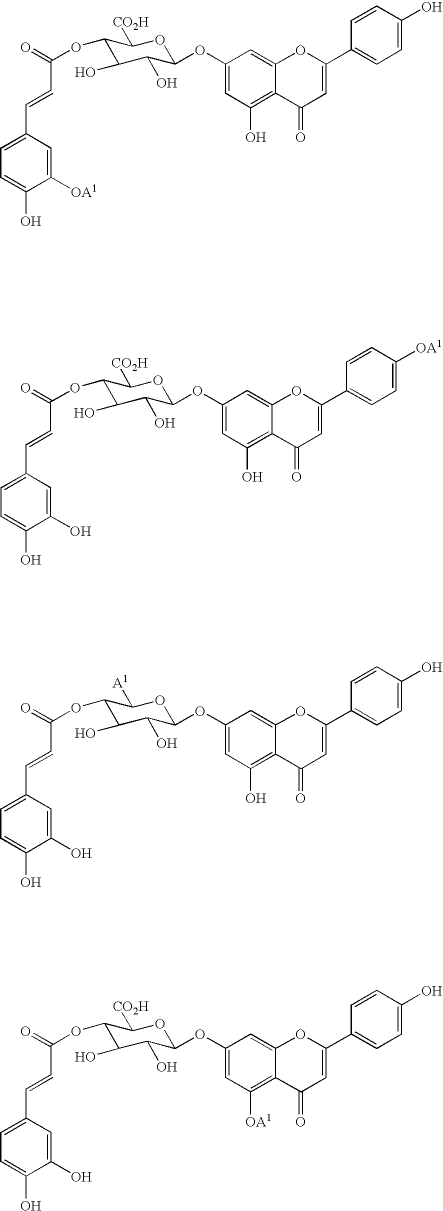
Group XXXIV

In one aspect, the invention includes phosphonate analogs of various sulfur-containing compounds including phosphonate analogs of: polyanionic sulfonate suramin and dextran sulfate (Billich et al (1992) Antivir. Chem. Chemother. 3:113-119; Carteau et al (1993) Arch. Biochem. Biophys. 305:606-610); diaryl sulfones (Gervay-Hague et al (2003) Abstracts of Papers, 225th ACS National Meeting, New Orleans, La., United States, Mar. 23-27, 2003; Abstract No. 2003:184008; Neamati et al (1997) Antimicrob. Agents Chemother. 41:385-393); sulfonamides (Nicklaus et al (1997) J. Med. Chem. 40:920-929); aromatic disulfides; and 2-mercaptobenzenesulfonamides (Neamati et al (1997) Antimicrob. Agents Chemother. 8:485-495).

Exemplary phosphonate sulfonamide Formula XXXIV compounds include:

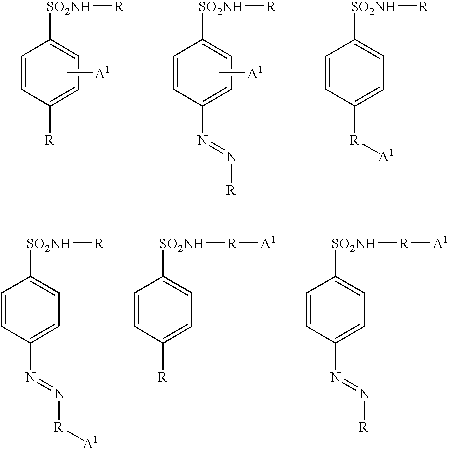

Exemplary diaryl sulfone phosphonate Formula XXXIV compounds include:

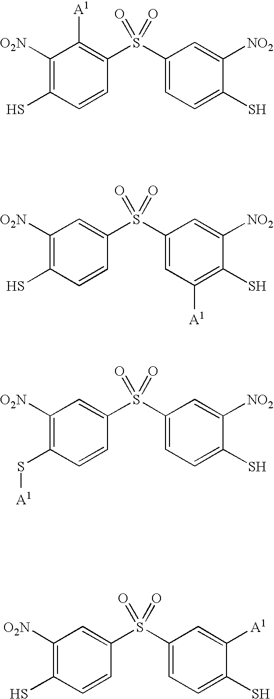

Exemplary distyryl disulfone phosphonate Formula XXXIV compounds include:

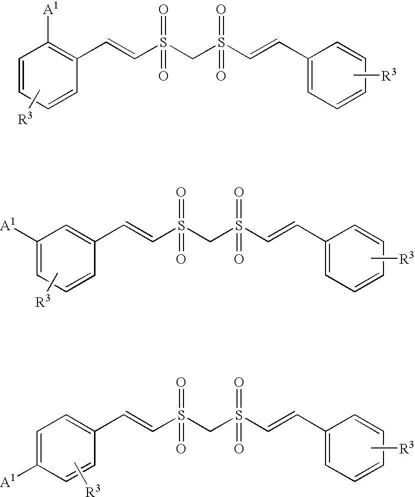

Exemplary 2-mercaptobenzenesulfonamide phosphonate Formula XXXIV compounds include the structures: 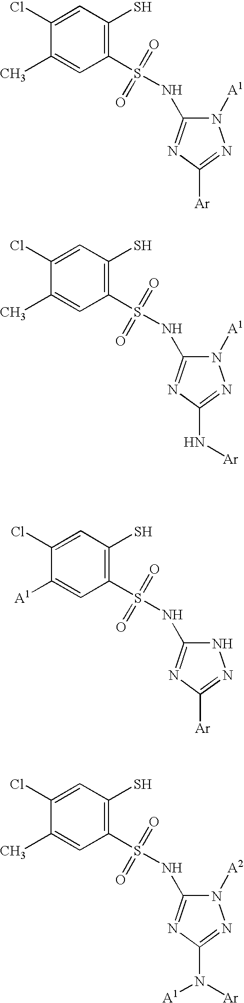
where Ar is carbocycle or heterocycle.
Group XXXV

In one aspect, the invention includes phosphonate analogs of symmetrical pentamidine compounds derived from serine protease inhibitors (WO 02/02516). Exemplary embodiments of pentamidine phosphonate Formula XXXV compounds include the structures:

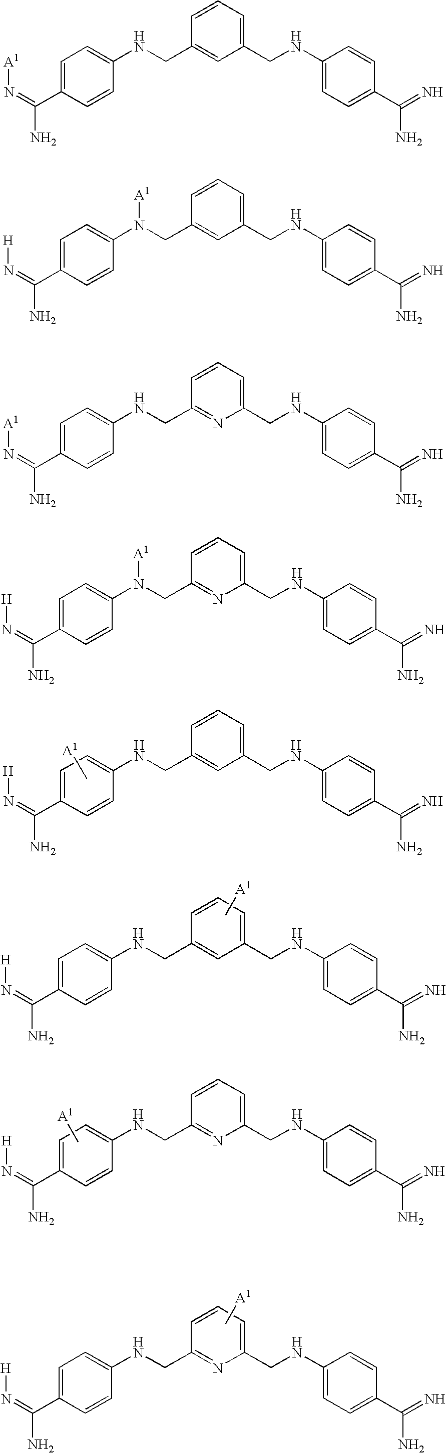

Exemplary embodiments of pentamidine phosphonate Formula XXXV compounds also include the structures:

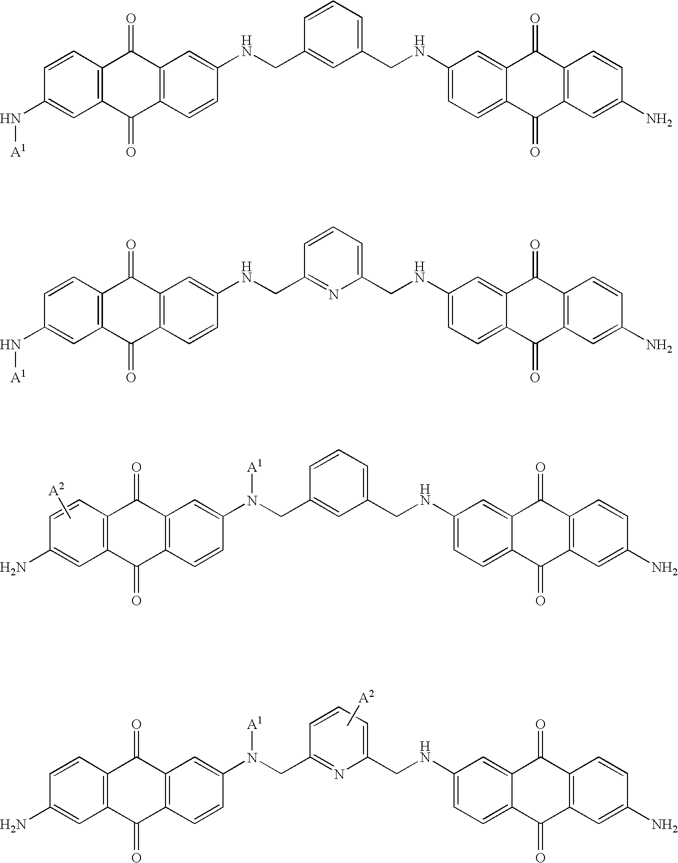

Exemplary embodiments of pentamidine phosphonate Formula XXXV compounds also include the structures:

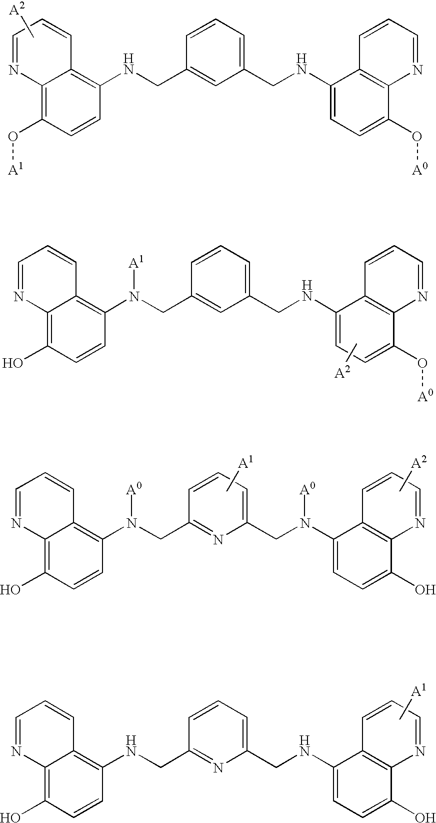

Exemplary embodiments of pentamidine phosphonate Formula XXXV compounds also include the structures: 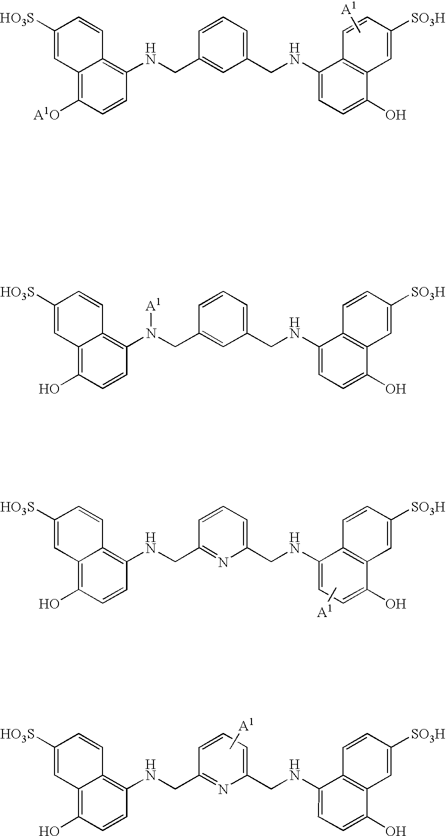
Group XXXVI

In one aspect, the invention includes phosphonate analogs of nucleic acid compounds. Nucleic acid phosphonate compounds include: (a) nucleosides and nucleotides (Zhao et al (1997) Heterocycles 45:2277-2282; Drake et al (1998) Proc. Natl. Acad. Sci. USA 95:4170-4175; Mazumder et al (1994) Proc. Natl. Acad. Sci. USA 91:5771-5775), dinucleotides (Taktakishvili et al (2000) J. Am. Chem. Soc. 122(24):5671-5677; Mazumder et al (1997) Mol. Pharmacol. 51:567-575) including 5H-pyrano[2,3-d:-6,5-d′]dipyrimidines (Pannecouque et al (2002) Current Biology 12(14):1169-1177); (b) oligonucleotides (Bischerour et al (2003) Nucleic Acids Research 31(10):2694-2702; Risitano et al (2003) Biochemistry 42(21):6507-6513; Guenther et al (2002) Bioorganic & Medicinal Chemistry Letters 12(16):2233-2236; de Soultrait et al (2002) Jour. of Mol. Biology 324(2):195-203; Jing et al (2002) Biochemistry 41(17):5397-5403; Jing et al (2000) J. Biol. Chem. 275(5):3421-3430; Jing et al (2000) J. Biol. Chem. 275(28):21460-21467; Jing et al (2001) DNA and Cell Biology 20(8):499-508; Snasel et al (2001) Eur. J. Biochem. 268(4):980-986; Mouscadet et al (1994) J. Biol. Chem. 269:21635-21638; Mazumder et al (1996) Biochemistry 35:13762-13771; Ojwang et al (1995) Antimicrob. Agents Chemother. 39:2426-2435; Thomas et al (1997) Trends Biotechnol. 15:167-172; Este et al (1998) Mol. Pharmacol. 53:340-345; Allen et al (1995) Virology 209:327-336; U.S. Pat. No. 6,323,185; U.S. Pat. No. 5,567,604); and (c) analogs thereof, with one or more phosphonate groups. Nucleic acid analogs include L and D stereoisomers (Mazumder et al (1996) Mol Pharmacol. 49:621-62); nucleobase analogs (Brodin et al (2002) Biochemistry 41(5):1529-1538; Brodin et al (2001) Nucleosides, Nucleotides & Nucleic Acids 20(4-7):481-486); sugar analogs and; internucleotide phosphate analogs (Tramontano et al (1998) Biochemistry 37:7237-7243; Zhang et al (1998) Bioorg. Med. Chem. Lett. 8:1887-1890; 8).

Embodiments of phosphonate analogs of nucleic acid HIV integrase inhibitor Formula XXXVI compounds include the structure: 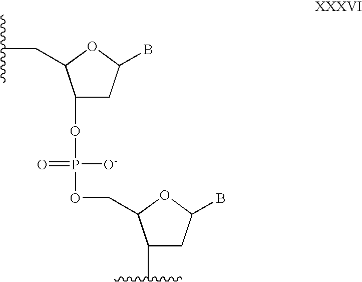
where the wavy lines indicate additional nucleotide units in the molecule and B is a nucleobase. Formula XXXVI compounds may be substituted at any location on the 5′ terminus, 3′ terminus, internucleotide phosphate linkage, sugar, or nucleobase moieties with a phosphonate group, as described for A¹. Formula XXXVI compounds also include any oligonucleotide analog with a modified internucleotide linkage, a modified sugar, or a modified nucleobase.
Group XXXVII

In one aspect, the invention includes phosphonate analogs of amino acids (WO 02/026697; U.S. Pat. No. 6,362,165) and peptides and proteins (Maroun et al (2001) Biochemistry 40(46): 13840-13848; Zhao et al (2003) Bioorganic & Medicinal Chemistry Letters 13(6): 1175-1177; Marchand et al (2003) Mol. Pharm. 64(3):600-609; Krajewski et al (2003) Bioorganic & Med. Chem. Letters 13(19):3203-3205; de Soultrait et al (2003) Current Medicinal Chem. 10(18):1765-1778; de Soultrait et al (2002) Jour. Mol. Biology 318(1):45-58; Sonika et al (2002) Journ. of Biomolecular Structure & Dynamics 20(1):31-38; Ng et al (2001) Life Sciences 69(19):2217-2223; Lutzke et al (1995) Proc. Natl. Acad. Sci USA 92:11456-11460; Krebs et al (1998) Eur. J. Biochem. 253:236-244; Sourgen et al (1996) Eur. J. Biochem. 240:765-773; Gulizia et al (1994) J. Virol. 68:2021-2025; WO 02/004488), including cyclic peptide (Singh et al (2001) Jour. Natural Products 64:874-882; Singh et al (2002) Organic Letters 4:1431-1434) and antibody (Strayer et al (2002) Molecular Therapy 5(1):33-41; Barsov et al (1996) J. Virology 70:4484-4494; U.S. 2003206909 A1; U.S. 2003152913 A1; U.S. 2003206909 A1) phosphonate compounds.

Embodiments of phosphonate analogs of peptide or protein HIV integrase inhibitor Formula XXXVII compounds include the structure: 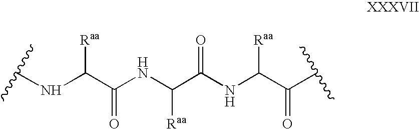
where the wavy lines indicate additional amino acid units in the molecule and R^{aa} is an amino acid side chain. Formula XXXVII compounds may be substituted at any location on the amino terminus, carboxyl terminus, side chain, or amide backbone with a phosphonate group, as described for A¹.

Exemplary phosphonate peptide and protein Formula XXXVII compounds include the substructures: 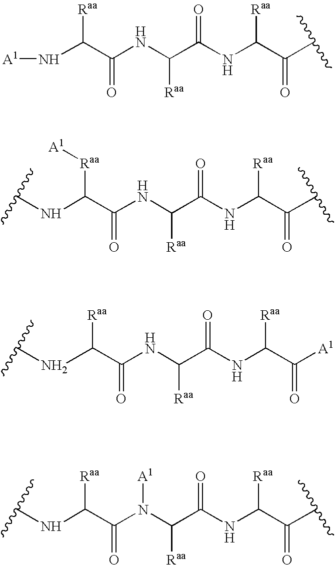
Group XXXVIII

In one aspect, the invention includes phosphonate analogs of polyketide natural products including Xanthoviridicatins isolated from a fermentation broth of an endophytic strain of Penicillium chrysogenum (Singh, et al (2003) Helvetica Chimica Acta, 86(10):3380-3385) having the Formula XXXVIII structure:

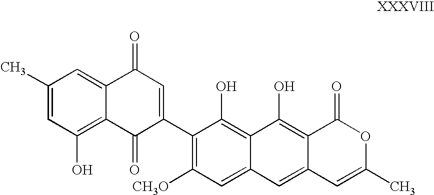

Exemplary phosphonate polyketide Formula XXXVIII compounds include: 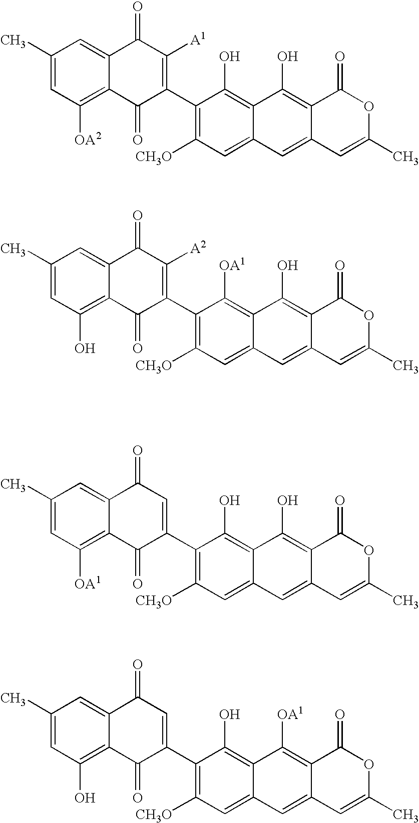
Group XXXIX

In one aspect, the invention includes phosphonate analogs of polyketide natural products including cytosporic acid, australifungin and australifunginol isolated from a fermentation broth of the filamentous fungus Cytospora sp. (Jayasuriya et al (2003) Journal of Natural Products 66(4):551-553).

Exemplary phosphonate cytosporic australifungin and australifunginol analog Formula XXXIX compounds include: 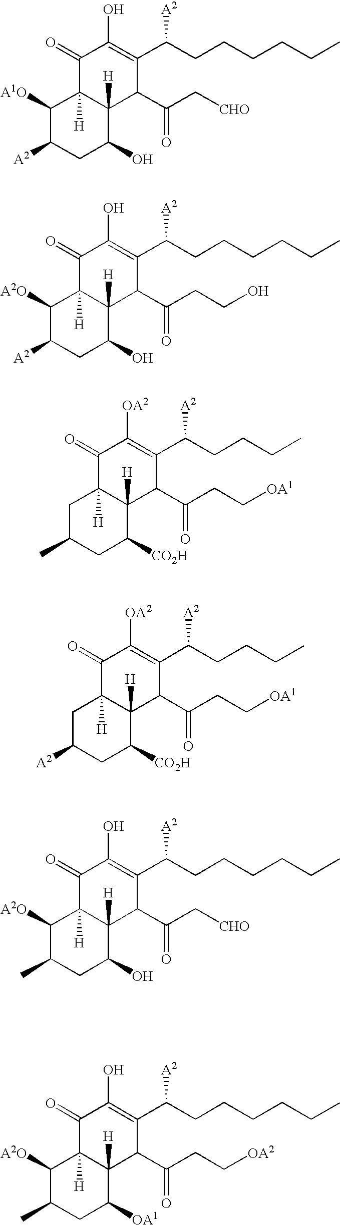
Protecting Groups

In the context of the present invention, embodiments of protecting groups include prodrug moieties and chemical protecting groups.

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protection. For example, protecting groups for —OH groups (whether hydroxyl, carboxylic acid, phosphonic acid, or other functions) are embodiments of “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in “Protective Groups in Organic Chemistry”, Theodora W. Greene (John Wiley & Sons, Inc., New York, 1991, ISBN 0-471-62301-6) (“Greene”). See also Kocienski, Philip J.; “Protecting Groups” (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below. Such groups include by way of example and not limitation, esters, amides, hydrazides, and the like.

Ether- and Ester-Forming Protecting Groups

Ester-forming groups include: (1) phosphonate ester-forming groups, such as phosphonamidate esters, phosphorothioate esters, phosphonate esters, and phosphon-bis-amidates; (2) carboxyl ester-forming groups, and (3) sulphur ester-forming groups, such as sulphonate, sulfate, and sulfinate.

The phosphonate moieties of the compounds of the invention may or may not be prodrug moieties, i.e. they may or may be susceptible to hydrolytic or enzymatic cleavage or modification. Certain phosphonate moieties are stable under most or nearly all metabolic conditions. For example, a dialkylphosphonate, where the alkyl groups are two or more carbons, may have appreciable stability in vivo due to a slow rate of hydrolysis. Within the context of phosphonate prodrug moieties, a large number of structurally-diverse prodrugs have been described for phosphonic acids (Freeman and Ross in Progress in Medicinal Chemistry 34: 112-147 (1997) and are included within the scope of the present invention.

In its ester-forming role, a protecting group typically is bound to any acidic group such as, by way of example and not limitation, a —CO₂H or —C(S)OH group, thereby resulting in —CO₂R^{x} where R^{x} is defined herein. Also, R^{x} for example includes the enumerated ester groups of WO 95/07920.

Examples of protecting groups include:

C₃-C₁₂ heterocycle (described above) or aryl. These aromatic groups optionally are polycyclic or monocyclic. Examples include phenyl, spiryl, 2- and 3-pyrrolyl, 2- and 3-thienyl, 2- and 4-imidazolyl, 2-, 4- and 5-oxazolyl, 3- and 4-isoxazolyl, 2-, 4- and 5-thiazolyl, 3-, 4- and 5-isothiazolyl, 3- and 4-pyrazolyl, 1-, 2-, 3- and 4-pyridinyl, and 1-, 2-, 4- and 5-pyrimidinyl,

C₃-C₁₂ heterocycle or aryl substituted with halo, R¹, R¹—O—C₁-C₁₂ alkylene, C₁-C₁₂ alkoxy, CN, NO₂, OH, carboxy, carboxyester, thiol, thioester, C₁-C₁₂ haloalkyl (1-6 halogen atoms), C₂-C₁₂ alkenyl or C₂-C₁₂ alkynyl. Such groups include 2-, 3- and 4-alkoxyphenyl (C₁-C₁₂ alkyl), 2-, 3- and 4-methoxyphenyl, 2-, 3- and 4-ethoxyphenyl, 2,3-, 2,4-, 2,5-, 2,6-, 3,4- and 3,5-diethoxyphenyl, 2- and 3-carboethoxy-4-hydroxyphenyl, 2- and 3-ethoxy-4-hydroxyphenyl, 2- and 3-ethoxy-5-hydroxyphenyl, 2- and 3-ethoxy-6-hydroxyphenyl, 2-, 3- and 4-O-acetylphenyl, 2-, 3- and 4-dimethylaminophenyl, 2-, 3- and 4-methylmercaptophenyl, 2-, 3- and 4-halophenyl (including 2-, 3- and 4-fluorophenyl and 2-, 3- and 4-chlorophenyl), 2,3-, 2,4-, 2,5-, 2,6-, 3,4- and 3,5-dimethylphenyl, 2,3-, 2,4-, 2,5-, 2,6-, 3,4- and 3,5-biscarboxyethylphenyl, 2,3-, 2,4-, 2,5-, 2,6-, 3,4- and 3,5-dimethoxyphenyl, 2,3-, 2,4-, 2,5-, 2,6-, 3,4- and 3,5-dihalophenyl (including 2,4-difluorophenyl and 3,5-difluorophenyl), 2-, 3- and 4-haloalkylphenyl (1 to 5 halogen atoms, C₁-C₁₂ alkyl including 4-trifluoromethylphenyl), 2-, 3- and 4-cyanophenyl, 2-, 3- and 4-nitrophenyl, 2-, 3- and 4-haloalkylbenzyl (1 to 5 halogen atoms, C₁-C₁₂ alkyl including 4-trifluoromethylbenzyl and 2-, 3- and 4-trichloromethylphenyl and 2-, 3- and 4-trichloromethylphenyl), 4-N-methylpiperidinyl, 3-N-methylpiperidinyl, 1-ethylpiperazinyl, benzyl, alkylsalicylphenyl (C₁-C₄ alkyl, including 2-, 3- and 4-ethylsalicylphenyl), 2-, 3- and 4-acetylphenyl, 1,8-dihydroxynaphthyl (—C₁₀H₆—OH) and aryloxy ethyl [C₆-C₉ aryl (including phenoxy ethyl)], 2,2′-dihydroxybiphenyl, 2-, 3- and 4-N,N-dialkylaminophenol, —C₆H₄CH₂—N(CH₃)₂, trimethoxybenzyl, triethoxybenzyl, 2-alkyl pyridinyl (C₁₋₄ alkyl); 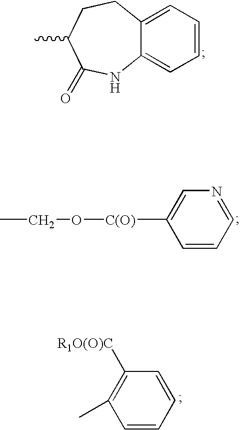
C₄-C₈ esters of 2-carboxyphenyl; and C₁-C₄ alkylene-C₃-C₆ aryl (including benzyl, —CH₂-pyrrolyl, —CH₂-thienyl, —CH₂-imidazolyl, —CH₂-oxazolyl, —CH₂-isoxazolyl, —CH₂-thiazolyl, —CH₂-isothiazolyl, —CH₂-pyrazolyl, —CH₂-pyridinyl and —CH₂-pyrimidinyl) substituted in the aryl moiety by 3 to 5 halogen atoms or 1 to 2 atoms or groups selected from halogen, C₁-C₁₂ alkoxy (including methoxy and ethoxy), cyano, nitro, OH, C₁-C₁₂ haloalkyl (1 to 6 halogen atoms; including —CH₂CCl₃), C₁-C₁₂ alkyl (including methyl and ethyl), C₂-C₁₂ alkenyl or C₂-C₁₂ alkynyl; alkoxy ethyl [C₁-C₆ alkyl including —CH₂—CH₂—O—CH₃ (methoxy ethyl)]; alkyl substituted by any of the groups set forth above for aryl, in particular OH or by 1 to 3 halo atoms (including —CH₃, —CH(CH₃)₂, —C(CH₃)₃, —CH₂CH₃, —(CH₂)₂CH₃, —(CH₂)₃CH₃, —(CH₂)₄CH₃, —(CH₂)₅CH₃, —CH₂CH₂F, —CH₂CH₂Cl, —CH₂CF₃, and —CH₂CCl₃); 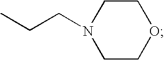
—N-2-propylmorpholino, 2,3-dihydro-6-hydroxyindene, sesamol, catechol monoester, —CH₂—C(O)—N(R¹)₂, —CH₂—S(O)(R¹), —CH₂—S(O)₂(R¹), —CH₂—CH(OC(O)CH₂R¹)—CH₂(OC(O)CH₂R¹), cholesteryl, enolpyruvate (HOOC—C(═CH₂)—), glycerol;

a 5 or 6 carbon monosaccharide, disaccharide or oligosaccharide (3 to 9 monosaccharide residues);

triglycerides such as α-D-β-diglycerides (wherein the fatty acids composing glyceride lipids generally are naturally occurring saturated or unsaturated C₆₋₂₆, C₆₋₁₈ or C₆₋₁₀ fatty acids such as linoleic, lauric, myristic, palmitic, stearic, oleic, palmitoleic, linolenic and the like fatty acids) linked to acyl of the parental compounds herein through a glyceryl oxygen of the triglyceride;

phospholipids linked to the carboxyl group through the phosphate of the phospholipid;

phthalidyl (shown in FIG. 1 of Clayton et al., Antimicrob. Agents Chemo. (1974) 5(6):670-671;

cyclic carbonates such as (5-R_{d}-2-oxo-1,3-dioxolen-4-yl) methyl esters (Sakamoto et al., Chem. Pharm. Bull. (1984) 32(6)2241-2248) where R_{d} is R₁, R₄ or aryl; and

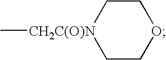

The hydroxyl groups of the compounds of this invention optionally are substituted with one of groups III, IV or V disclosed in WO 94/21604, or with isopropyl.

As further embodiments, Table A lists examples of protecting group ester moieties that for example can be bonded via oxygen to —C(O)O— and —P(O)(O—)₂ groups. Several amidates also are shown, which are bound directly to —C(O)— or —P(O)₂. Esters of structures 1-5, 8-10 and 16, 17, 19-22 are synthesized by reacting the compound herein having a free hydroxyl with the corresponding halide (chloride or acyl chloride and the like) and N,N-dicyclohexyl-N-morpholine carboxamidine (or another base such as DBU, triethylamine, CsCO₃, N,N-dimethylaniline and the like) in DMF (or other solvent such as acetonitrile or N-methylpyrrolidone). When the compound to be protected is a phosphonate, the esters of structures 5-7, 11, 12, 21, and 23-26 are synthesized by reaction of the alcohol or alkoxide salt (or the corresponding amines in the case of compounds such as 13, 14 and 15) with the monochlorophosphonate or dichlorophosphonate (or another activated phosphonate).

TABLE A
1. —CH₂—C(O)—N(R₁)₂*
2. —CH₂—S(O)(R₁)
3. —CH₂—S(O)₂(R₁)
4. —CH₂—O—C(O)—CH₂—C₆H₅
5. 3-cholesteryl
6. 3-pyridyl
7. N-ethylmorpholino
8. —CH₂—O—C(O)—C₆H₅
9. —CH₂—O—C(O)—CH₂CH₃CH₂(OC(O)CH₂R₁)*
10. —CH₂—O—C(O)—C(CH₃)₃
11. —CH₂—CCl₃
12. —C₆H₅
13. —NH—CH₂—C(O)O—CH₂CH₃
14. —N(CH₃)—CH₂—C(O)O—CH₂CH₃
15. —NHR₁
16. —CH₂—O—C(O)—C₁₀H₁₅
17. —CH₂—O—C(O)—CH(CH₃)₂
18. —CH₂—C#H(OC(O)CH₂R₁)—

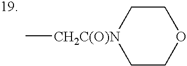

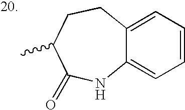

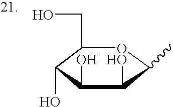

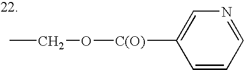

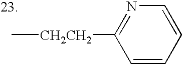

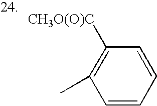

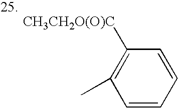

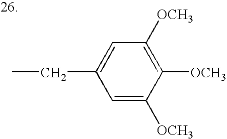

#—chiral center is (R), (S) or racemate.

Other esters that are suitable for use herein are described in EP 632048.

Protecting groups also includes “double ester” forming profunctionalities such as —CH₂OC(O)OCH₃, 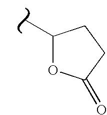
—CH₂SCOCH₃, —CH₂OCON(CH₃)₂, or alkyl- or aryl-acyloxyalkyl groups of the structure —CH(R¹ or W⁵)O((CO)R³⁷) or —CH(R¹ or W⁵)((CO)OR³⁸) (linked to oxygen of the acidic group) wherein R³⁷ and R³⁸ are alkyl, aryl, or alkylaryl groups (see U.S. Pat. No. 4,968,788). Frequently R³⁷ and R³⁸ are bulky groups such as branched alkyl, ortho-substituted aryl, meta-substituted aryl, or combinations thereof, including normal, secondary, iso- and tertiary alkyls of 1-6 carbon atoms. An example is the pivaloyloxymethyl group. These are of particular use with prodrugs for oral administration. Examples of such useful protecting groups are alkylacyloxymethyl esters and their derivatives, including —CH(CH₂CH₂OCH₃)OC(O)C(CH₃)₃, 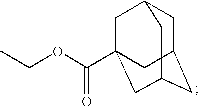
—CH₂OC(O)C₁₀H₁₅, —CH₂OC(O)C(CH₃)₃, —CH(CH₂OCH₃)OC(O)C(CH₃)₃, —CH(CH(CH₃)₂)OC(O)C(CH₃)₃, —CH₂OC(O)CH₂CH(CH₃)₂, —CH₂OC(O)C₆H₁₁, —CH₂OC(O)C₆H₅, —CH₂OC(O)C₁₀H₁₅, —CH₂OC(O)CH₂CH₃, —CH₂OC(O)CH(CH₃)₂, —CH₂OC(O)C(CH₃)₃ and —CH₂OC(O)CH₂C₆H₅.

For prodrug purposes, the ester typically chosen is one heretofore used for antibiotic drugs, in particular the cyclic carbonates, double esters, or the phthalidyl, aryl or alkyl esters.

In some embodiments the protected acidic group is an ester of the acidic group and is the residue of a hydroxyl-containing functionality. In other embodiments, an amino compound is used to protect the acid functionality. The residues of suitable hydroxyl or amino-containing functionalities are set forth above or are found in WO 95/07920. Of particular interest are the residues of amino acids, amino acid esters, polypeptides, or aryl alcohols. Typical amino acid, polypeptide and carboxyl-esterified amino acid residues are described on pages 11-18 and related text of WO 95/07920 as groups L1 or L2. WO 95/07920 expressly teaches the amidates of phosphonic acids, but it will be understood that such amidates are formed with any of the acid groups set forth herein and the amino acid residues set forth in WO 95/07920.

Typical esters for protecting acidic functionalities are also described in WO 95/07920, again understanding that the same esters can be formed with the acidic groups herein as with the phosphonate of the '920 publication. Typical ester groups are defined at least on WO 95/07920 pages 89-93 (under R³¹ or R³⁵), the table on page 105, and pages 21-23 (as R). Of particular interest are esters of unsubstituted aryl such as phenyl or arylalkyl such benzyl, or hydroxy-, halo-, alkoxy-, carboxy- and/or alkylestercarboxy-substituted aryl or alkylaryl, especially phenyl, ortho-ethoxyphenyl, or C₁-C₄ alkylestercarboxyphenyl (salicylate C₁-C₁₂ alkylesters).

The protected acidic groups, particularly when using the esters or amides of WO 95/07920, are useful as prodrugs for oral administration. However, it is not essential that the acidic group be protected in order for the compounds of this invention to be effectively administered by the oral route. When the compounds of the invention having protected groups, in particular amino acid amidates or substituted and unsubstituted aryl esters are administered systemically or orally they are capable of hydrolytic cleavage in vivo to yield the free acid.

One or more of the acidic hydroxyls are protected. If more than one acidic hydroxyl is protected then the same or a different protecting group is employed, e.g., the esters may be different or the same, or a mixed amidate and ester may be used.

Typical hydroxy protecting groups described in Greene (pages 14-118) include substituted methyl and alkyl ethers, substituted benzyl ethers, silyl ethers, esters including sulfonic acid esters, and carbonates.

Exemplary hydroxy protecting groups include:

Ethers (methyl, t-butyl, allyl);
Substituted Methyl Ethers (Methoxymethyl, Methylthiomethyl, t-Butylthiomethyl, (Phenyldimethylsilyl)methoxymethyl, Benzyloxymethyl, p-Methoxybenzyloxymethyl, (4-Methoxyphenoxy)methyl, Guaiacolmethyl, t-Butoxymethyl, 4-Pentenyloxymethyl, Siloxymethyl, 2-Methoxyethoxymethyl, 2,2,2-Trichloroethoxymethyl, Bis(2-chloroethoxy)methyl, 2-(Trimethylsilyl)ethoxymethyl, Tetrahydropyranyl, 3-Bromotetrahydropyranyl, Tetrahydropthiopyranyl, 1-Methoxycyclohexyl, 4-Methoxytetrahydropyranyl, 4-Methoxytetrahydrothiopyranyl, 4-Methoxytetrahydropthiopyranyl S,S-Dioxido, 1-[(2-Chloro-4-methyl)phenyl]-4-methoxypiperidin-4-yl, 1,4-Dioxan-2-yl, Tetrahydrofuranyl, Tetrahydrothiofuranyl, 2,3,3a,4,5,6,7,7a-Octahydro-7,8,8-trimethyl-4,7-methanobenzofuran-2-yl));
Substituted Ethyl Ethers (1-Ethoxyethyl, 1-(2-Chloroethoxy)ethyl, 1-Methyl-1-methoxyethyl, 1-Methyl-1-benzyloxyethyl, 1-Methyl-1-benzyloxy-2-fluoroethyl, 2,2,2-Trichloroethyl, 2-Trimethylsilylethyl, 2-(Phenylselenyl)ethyl,
p-Chlorophenyl, p-Methoxyphenyl, 2,4-Dinitrophenyl, Benzyl);
Substituted Benzyl Ethers (p-Methoxybenzyl, 3,4-Dimethoxybenzyl, o-Nitrobenzyl, p-Nitrobenzyl, p-Halobenzyl, 2,6-Dichlorobenzyl, p-Cyanobenzyl, p-Phenylbenzyl, 2- and 4-Picolyl, 3-Methyl-2-picolyl N-Oxido, Diphenylmethyl, p,p′-Dinitrobenzhydryl, 5-Dibenzosuberyl, Triphenylmethyl, α-Naphthyldiphenylmethyl, p-methoxyphenyldiphenylmethyl, Di(p-methoxyphenyl)phenylmethyl, Tri(p-methoxyphenyl)methyl, 4-(4′-Bromophenacyloxy)phenyldiphenylmethyl, 4,4′,4″-Tris(4,5-dichlorophthalimidophenyl)methyl, 4,4′,4″-Tris(levulinoyloxyphenyl)methyl, 4,4′,4″-Tris(benzoyloxyphenyl)methyl, 3-(Imidazol-1-ylmethyl)bis(4′,4″-dimethoxyphenyl)methyl, 1,1-Bis(4-methoxyphenyl)-1′-pyrenylmethyl, 9-Anthryl, 9-(9-Phenyl)xanthenyl, 9-(9-Phenyl-10-oxo)anthryl, 1,3-Benzodithiolan-2-yl, Benzisothiazolyl S,S-Dioxido);
Silyl Ethers (Trimethylsilyl, Triethylsilyl, Triisopropylsilyl, Dimethylisopropylsilyl, Diethylisopropylsilyl, Dimethylthexylsilyl, t-Butyldimethylsilyl, t-Butyldiphenylsilyl, Tribenzylsilyl, Tri-p-xylylsilyl, Triphenylsilyl, Diphenylmethylsilyl, t-Butylmethoxyphenylsilyl);
Esters (Formate, Benzoylformate, Acetate, Choroacetate, Dichloroacetate, Trichloroacetate, Trifluoroacetate, Methoxyacetate, Triphenylmethoxyacetate, Phenoxyacetate, p-Chlorophenoxyacetate, p-poly-Phenylacetate, 3-Phenylpropionate, 4-Oxopentanoate (Levulinate), 4,4-(Ethylenedithio)pentanoate, Pivaloate, Adamantoate, Crotonate, 4-Methoxycrotonate, Benzoate, p-Phenylbenzoate, 2,4,6-Trimethylbenzoate (Mesitoate));
Carbonates (Methyl, 9-Fluorenylmethyl, Ethyl, 2,2,2-Trichloroethyl, 2-(Trimethylsilyl)ethyl, 2-(Phenylsulfonyl)ethyl, 2-(Triphenylphosphonio)ethyl, Isobutyl, Vinyl, Allyl, p-Nitrophenyl, Benzyl, p-Methoxybenzyl, 3,4-Dimethoxybenzyl, o-Nitrobenzyl, p-Nitrobenzyl, S-Benzyl Thiocarbonate, 4-Ethoxy-1-naphthyl, Methyl Dithiocarbonate);
Groups With Assisted Cleavage (2-Iodobenzoate, 4-Azidobutyrate, 4-Nitro-4-methylpentanoate, o-(Dibromomethyl)benzoate, 2-Formylbenzenesulfonate, 2-(Methylthiomethoxy)ethyl Carbonate, 4-(Methylthiomethoxy)butyrate, 2-(Methylthiomethoxymethyl)benzoate); Miscellaneous Esters (2,6-Dichloro-4-methylphenoxyacetate, 2,6-Dichloro-4-(1,1,3,3 tetramethylbutyl)phenoxyacetate, 2,4-Bis(1,1-dimethylpropyl)phenoxyacetate, Chlorodiphenylacetate, Isobutyrate, Monosuccinate, (E)-2-Methyl-2-butenoate (Tigloate), o-(Methoxycarbonyl)benzoate, p-poly-Benzoate, α-Naphthoate, Nitrate, Alkyl N,N,N′,N′-Tetramethylphosphorodiamidate, N-Phenylcarbamate, Borate, Dimethylphosphinothioyl, 2,4-Dinitrophenylsulfenate); and
Sulfonates (Sulfate, Methanesulfonate (Mesylate), Benzylsulfonate, Tosylate).

Typical 1,2-diol protecting groups (thus, generally where two OH groups are taken together with the protecting functionality) are described in Greene at pages 118-142 and include Cyclic Acetals and Ketals (Methylene, Ethylidene, 1-t-Butylethylidene, 1-Phenylethylidene, (4-Methoxyphenyl)ethylidene, 2,2,2-Trichloroethylidene, Acetonide (Isopropylidene), Cyclopentylidene, Cyclohexylidene, Cycloheptylidene, Benzylidene, p-Methoxybenzylidene, 2,4-Dimethoxybenzylidene, 3,4-Dimethoxybenzylidene, 2-Nitrobenzylidene); Cyclic Ortho Esters (Methoxymethylene, Ethoxymethylene, Dimethoxymethylene, 1-Methoxyethylidene, 1-Ethoxyethylidine, 1,2-Dimethoxyethylidene, α-Methoxybenzylidene, 1-(N,N-Dimethylamino)ethylidene Derivative, α-(N,N-Dimethylamino)benzylidene Derivative, 2-Oxacyclopentylidene); Silyl Derivatives (Di-t-butylsilylene Group, 1,3-(1,1,3,3-Tetraisopropyldisiloxanylidene), and Tetra-t-butoxydisiloxane-1,3-diylidene), Cyclic Carbonates, Cyclic Boronates, Ethyl Boronate and Phenyl Boronate.

More typically, 1,2-diol protecting groups include those shown in Table B, still more typically, epoxides, acetonides, cyclic ketals and aryl acetals.

TABLE B

| | | | | |
|---|---|---|---|---|
| 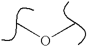 | 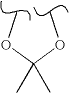 | 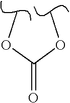 | 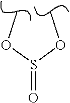 | 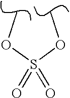 |

| | | | |
|---|---|---|---|
| 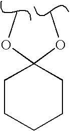 | 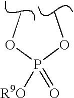 | 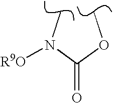 | 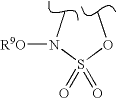 |

| | |
|---|---|
| 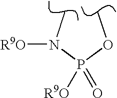 | 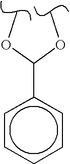 |

wherein R⁹ is C₁-C₆ alkyl.
Amino Protecting Groups

Another set of protecting groups include any of the typical amino protecting groups described by Greene at pages 315-385.

Exemplary amino protecting groups include:

Carbamates: (methyl and ethyl, 9-fluorenylmethyl, 9(2-sulfo)fluorenylmethyl, 9-(2,7-dibromo)fluorenylmethyl, 2,7-di-t-butyl-[9-(10,10-dioxo-10,10,10,10-tetrahydrothioxanthyl)]methyl, 4-methoxyphenacyl);
Substituted Ethyl: (2,2,2-trichoroethyl, 2-trimethylsilylethyl, 2-phenylethyl, 1-(1-adamantyl)-1-methylethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-t-butylphenyl)-1-methylethyl, 2-(2′- and 4′-pyridyl)ethyl, 2-(N,N-dicyclohexylcarboxamido)ethyl, t-butyl, 1-adamantyl, vinyl, allyl, 1-isopropylallyl, cinnamyl, 4-nitrocinnamyl, 8-quinolyl, N-hydroxypiperidinyl, alkyldithio, benzyl, p-methoxybenzyl, p-nitrobenzyl, p-bromobenzyl, p-chlorobenzyl, 2,4-dichlorobenzyl, 4-methylsulfinylbenzyl, 9-anthrylmethyl, diphenylmethyl);
Groups With Assisted Cleavage: (2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 2-triphenylphosphonioisopropyl, 1,1-dimethyl-2-cyanoethyl, m-choro-p-acyloxybenzyl, p-(dihydroxyboryl)benzyl, 5-benzisoxazolylmethyl, 2-(trifluoromethyl)-6-chromonylmethyl);
Groups Capable of Photolytic Cleavage: (m-nitrophenyl, 3,5-dimethoxybenzyl, o-nitrobenzyl, 3,4-dimethoxy-6-nitrobenzyl, phenyl(o-nitrophenyl)methyl); Urea-Type Derivatives (phenothiazinyl-(10)-carbonyl, N′-p-toluenesulfonylaminocarbonyl, N′-phenylaminothiocarbonyl);
Miscellaneous Carbamates: (t-amyl, S-benzyl thiocarbamate, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N-dimethylcarboxamido)benzyl, 1,1-dimethyl-3-(N,N-dimethylcarboxamido)propyl, 1,1-dimethylpropynyl, di(2-pyridyl)methyl, 2-furanylmethyl, 2-Iodoethyl, Isobornyl, Isobutyl, Isonicotinyl, p-(p′-Methoxyphenylazo)benzyl, 1-methylcyclobutyl, 1-methylcyclohexyl, 1-methyl-1-cyclopropylmethyl, 1-methyl-1-(3,5-dimethoxyphenyl)ethyl, 1-methyl-1-(p-phenylazophenyl)ethyl, 1-methyl-1-phenylethyl, 1-methyl-1-(4-pyridyl)ethyl, phenyl, p-(phenylazo)benzyl, 2,4,6-tri-t-butylphenyl, 4-(trimethylammonium)benzyl, 2,4,6-trimethylbenzyl);
Amides: (N-formyl, N-acetyl, N-choroacetyl, N-trichoroacetyl, N-trifluoroacetyl, N-phenylacetyl, N-3-phenylpropionyl, N-picolinoyl, N-3-pyridylcarboxamide, N-benzoylphenylalanyl, N-benzoyl, N-p-phenylbenzoyl);
Amides With Assisted Cleavage: (N-o-nitrophenylacetyl, N-o-nitrophenoxyacetyl, N-acetoacetyl, (N′-dithiobenzyloxycarbonylamino)acetyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-3-methyl-3-nitrobutyryl, N-o-nitrocinnamoyl, N-acetylmethionine, N-o-nitrobenzoyl, N-o-(benzoyloxymethyl)benzoyl, 4,5-diphenyl-3-oxazolin-2-one);
Cyclic Imide Derivatives: (N-phthalimide, N-dithiasuccinoyl, N-2,3-diphenylmaleoyl, N-2,5-dimethylpyrrolyl, N-1,1,4,4-tetramethyldisilylazacyclopentane adduct, 5-substituted 1,3-dimethyl-1,3,5-triazacyclohexan-2-one, 5-substituted 1,3-dibenzyl-1,3-5-triazacyclohexan-2-one, 1-substituted 3,5-dinitro-4-pyridonyl);
N-Alkyl and N-Aryl Amines: (N-methyl, N-allyl, N-[2-(trimethylsilyl)ethoxy]methyl, N-3-acetoxypropyl, N-(1-isopropyl-4-nitro-2-oxo-3-pyrrolin-3-yl), Quaternary Ammonium Salts, N-benzyl, N-di(4-methoxyphenyl)methyl, N-5-dibenzosuberyl, N-triphenylmethyl, N-(4-methoxyphenyl)diphenylmethyl, N-9-phenylfluorenyl, N-2,7-dichloro-9-fluorenylmethylene, N-ferrocenylmethyl, N-2-picolylamine N′-oxide);
Imine Derivatives: (N-1,1-dimethylthiomethylene, N-benzylidene, N-p-methoxybenylidene, N-diphenylmethylene, N-[(2-pyridyl)mesityl]methylene, N,(N′,N′-dimethylaminomethylene, N,N-isopropylidene, N-p-nitrobenzylidene, N-salicylidene, N-5-chlorosalicylidene, N-(5-chloro-2-hydroxyphenyl)phenylmethylene, N-cyclohexylidene);
Enamine Derivatives: (N-(5,5-dimethyl-3-oxo-1-cyclohexenyl));
N-Metal Derivatives (N-borane derivatives, N-diphenylborinic acid derivatives, N-[phenyl(pentacarbonylchromium- or -tungsten)]carbenyl, N-copper or N-zinc chelate);
N-N Derivatives: (N-nitro, N-nitroso, N-oxide);
N-P Derivatives: (N-diphenylphosphinyl, N-dimethylthiophosphinyl, N-diphenylthiophosphinyl, N-dialkyl phosphoryl, N-dibenzyl phosphoryl, N-diphenyl phosphoryl);
N-Si Derivatives, N-S Derivatives, and N-Sulfenyl Derivatives: (N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, N-triphenylmethylsulfenyl, N-3-nitropyridinesulfenyl); and N-sulfonyl Derivatives (N-p-toluenesulfonyl, N-benzenesulfonyl, N-2,3,6-trimethyl-4-methoxybenzenesulfonyl, N-2,4,6-trimethoxybenzenesulfonyl, N-2,6-dimethyl-4-methoxybenzenesulfonyl, N-pentamethylbenzenesulfonyl, N-2,3,5,6,-tetramethyl-4-methoxybenzenesulfonyl, N-4-methoxybenzenesulfonyl, N-2,4,6-trimethylbenzenesulfonyl, N-2,6-dimethoxy-4-methylbenzenesulfonyl, N-2,2,5,7,8-pentamethylchroman-6-sulfonyl, N-methanesulfonyl, N-β-trimethylsilyethanesulfonyl, N-9-anthracenesulfonyl, N-4-(4′,8′-dimethoxynaphthylmethyl)benzenesulfonyl, N-benzylsulfonyl, N-trifluoromethylsulfonyl, N-phenacylsulfonyl).

More typically, protected amino groups include carbamates and amides, still more typically, —NHC(O)R¹ or —N═CR¹N(R¹)₂. Another protecting group, also useful as a prodrug for amino or —NH(R⁵), is: 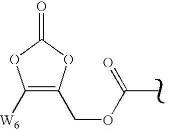
See for example Alexander, J. et al (1996) J. Med. Chem. 39:480-486.
Amino Acid and Polypeptide Protecting Group and Conjugates

An amino acid or polypeptide protecting group of a compound of the invention has the structure R¹⁵ NHCH(R¹⁶)C(O)—, where R¹⁵ is H, an amino acid or polypeptide residue, or R⁵, and R¹⁶ is defined below.

R¹⁶ is lower alkyl or lower alkyl (C₁-C₆) substituted with amino, carboxyl, amide, carboxyl ester, hydroxyl, C₆-C₇ aryl, guanidinyl, imidazolyl, indolyl, sulfhydryl, sulfoxide, and/or alkylphosphate. R¹⁰ also is taken together with the amino acid α N to form a proline residue (R¹⁰=—CH₂)₃—). However, R¹⁰ is generally the side group of a naturally-occurring amino acid such as H, —CH₃, —CH(CH₃)₂, —CH₂—CH(CH₃)₂, —CHCH₃—CH₂—CH₃, —CH₂—C₆H₅, —CH₂CH₂—S—CH₃, —CH₂OH, —CH(OH)—CH₃, —CH₂—SH, —CH₂—C₆H₄OH, —CH₂—CO—NH₂, —CH₂—CH₂—CO—NH₂, —CH₂—COOH, —CH₂—CH₂—COOH, —(CH₂)₄—NH₂ and —(CH₂)₃—NH—C(NH₂)—NH₂. R¹⁰ also includes 1-guanidinoprop-3-yl, benzyl, 4-hydroxybenzyl, imidazol-4-yl, indol-3-yl, methoxyphenyl and ethoxyphenyl.

Another set of protecting groups include the residue of an amino-containing compound, in particular an amino acid, a polypeptide, a protecting group, —NHSO₂R, NHC(O)R, —N(R)₂, NH₂ or —NH(R)(H), whereby for example a carboxylic acid is reacted, i.e. coupled, with the amine to form an amide, as in C(O)NR₂. A phosphonic acid may be reacted with the amine to form a phosphonamidate, as in —P(O)(OR)(NR₂).

In general, amino acids have the structure R¹⁷C(O)CH(R¹⁶)NH—, where R¹⁷ is —OH, —OR, an amino acid or a polypeptide residue. Amino acids are low molecular weight compounds, on the order of less than about 1000 MW and which contain at least one amino or imino group and at least one carboxyl group. Generally the amino acids will be found in nature, i.e., can be detected in biological material such as bacteria or other microbes, plants, animals or man. Suitable amino acids typically are alpha amino acids, i.e. compounds characterized by one amino or imino nitrogen atom separated from the carbon atom of one carboxyl group by a single substituted or unsubstituted alpha carbon atom. Of particular interest are hydrophobic residues such as mono- or di-alkyl or aryl amino acids, cycloalkylamino acids and the like. These residues contribute to cell permeability by increasing the partition coefficient of the parental drug. Typically, the residue does not contain a sulfhydryl or guanidino substituent.

Naturally-occurring amino acid residues are those residues found naturally in plants, animals or microbes, especially proteins thereof. Polypeptides most typically will be substantially composed of such naturally-occurring amino acid residues. These amino acids are glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine and hydroxyproline. Additionally, unnatural amino acids, for example, valanine, phenylglycine and homoarginine are also included. Commonly encountered amino acids that are not gene-encoded may also be used in the present invention. All of the amino acids used in the present invention may be either the D- or L-optical isomer. In addition, other peptidomimetics are also useful in the present invention. For a general review, see Spatola, A. F., in Chemistry and Biochemistry of Amino Acids, Peptides and Proteins, B. Weinstein, eds., Marcel Dekker, New York, p. 267 (1983).

When protecting groups are single amino acid residues or polypeptides, these conjugates may be produced by forming an amide bond between a carboxyl group of the amino acid (or C-terminal amino acid of a polypeptide for example). Generally, only one of any site in the parental molecule is amidated with an amino acid as described herein, although it is within the scope of this invention to introduce amino acids at more than one permitted site. In general, the α-amino or α-carboxyl group of the amino acid or the terminal amino or carboxyl group of a polypeptide are bonded to the parental functionalities, i.e., carboxyl or amino groups in the amino acid side chains generally are not used to form the amide bonds with the parental compound (although these groups may need to be protected during synthesis of the conjugates as described further below).

With respect to the carboxyl-containing side chains of amino acids or polypeptides it will be understood that the carboxyl group optionally will be blocked, e.g. by R¹, esterified with R⁵ or amidated. Similarly, the amino side chains R¹⁶ optionally will be blocked with R¹ or substituted with R⁵.

Such ester or amide bonds with side chain amino or carboxyl groups, like the esters or amides with the parental molecule, optionally are hydrolyzable in vivo or in vitro under acidic (pH <3) or basic (pH >10) conditions. Alternatively, they are substantially stable in the gastrointestinal tract of humans but are hydrolyzed enzymatically in blood or in intracellular environments. The esters or amino acid or polypeptide amidates also are useful as intermediates for the preparation of the parental molecule containing free amino or carboxyl groups. The free acid or base of the parental compound, for example, is readily formed from the esters or amino acid or polypeptide conjugates of this invention by conventional hydrolysis procedures.

When an amino acid residue contains one or more chiral centers, any of the D, L, meso, threo or erythro (as appropriate) racemates, scalemates or mixtures thereof may be used. In general, if the intermediates are to be hydrolyzed non-enzymatically (as would be the case where the amides are used as chemical intermediates for the free acids or free amines), D isomers are useful. On the other hand, L isomers are more versatile since they can be susceptible to both non-enzymatic and enzymatic hydrolysis, and are more efficiently transported by amino acid or dipeptidyl transport systems in the gastrointestinal tract.

Examples of suitable amino acids whose residues are represented by R^{x} or R^{y} include the following:

Glycine;

Aminopolycarboxylic acids, e.g., aspartic acid, β-hydroxyaspartic acid, glutamic acid, β-hydroxyglutamic acid, β-methylaspartic acid, β-methylglutamic acid, β,β-dimethylaspartic acid, γ-hydroxyglutamic acid, 1, γ-dihydroxyglutamic acid, 1-phenylglutamic acid, γ-methyleneglutamic acid, 3-aminoadipic acid, 2-aminopimelic acid, 2-aminosuberic acid and 2-aminosebacic acid;

Amino acid amides such as glutamine and asparagine;

Polyamino- or polybasic-monocarboxylic acids such as arginine, lysine, 1-aminoalanine, γ-aminobutyrine, ornithine, citruline, homoarginine, homocitrulline, hydroxylysine, allohydroxylsine and diaminobutyric acid;

Other basic amino acid residues such as histidine;

Diaminodicarboxylic acids such as α,α′-diaminosuccinic acid, α,α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid;

Imino acids such as proline, hydroxyproline, allohydroxyproline, γ-methylproline, pipecolic acid, 5-hydroxypipecolic acid, and azetidine-2-carboxylic acid;

A mono- or di-alkyl (typically C₁-C₈ branched or normal) amino acid such as alanine, valine, leucine, allylglycine, butyrine, norvaline, norleucine, heptyline, α-methylserine, α-amino-α-methyl-γ-hydroxyvaleric acid, α-amino-α-methyl-δ-hydroxyvaleric acid, α-amino-α-methyl-F-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, α-aminodiisoamyacetic acid, α-methylaspartic acid, α-methylglutamic acid, 1-aminocyclopropane-1-carboxylic acid, isoleucine, alloisoleucine, tert-leucine, β-methyltryptophan and α-amino-β-ethyl-β-phenylpropionic acid;

β-phenylserinyl;

Aliphatic α-amino-β-hydroxy acids such as serine, β-hydroxyleucine, β-hydroxynorleucine, β-hydroxynorvaline, and α-amino-β-hydroxystearic acid;

α-Amino, α-, γ-, δ- or ε-hydroxy acids such as homoserine, δ-hydroxynorvaline, γ-hydroxynorvaline and ε-hydroxynorleucine residues; canavine and canaline; γ-hydroxyomithine;

2-hexosaminic acids such as D-glucosaminic acid or D-galactosaminic acid;

α-Amino-β-thiols such as penicillamine, β-thiolnorvaline or β-thiolbutyrine;

Other sulfur containing amino acid residues including cysteine; homocystine, β-phenylmethionine, methionine, S-allyl-L-cysteine sulfoxide, 2-thiolhistidine, cystathionine, and thiol ethers of cysteine or homocysteine;

Phenylalanine, tryptophan and ring-substituted α-amino acids such as the phenyl- or cyclohexylamino acids α-aminophenylacetic acid, α-aminocyclohexylacetic acid and α-amino-β-cyclohexylpropionic acid; phenylalanine analogues and derivatives comprising aryl, lower alkyl, hydroxy, guanidino, oxyalkylether, nitro, sulfur or halo-substituted phenyl (e.g., tyrosine, methyltyrosine and o-chloro-, p-chloro-, 3,4-dichloro, o-, m- or p-methyl-, 2,4,6-trimethyl-, 2-ethoxy-5-nitro-, 2-hydroxy-5-nitro- and p-nitro-phenylalanine); furyl-, thienyl-, pyridyl-, pyrimidinyl-, purinyl- or naphthyl-alanines; and tryptophan analogues and derivatives including kynurenine, 3-hydroxykynurenine, 2-hydroxytryptophan and 4-carboxytryptophan;

α-Amino substituted amino acids including sarcosine (N-methylglycine), N-benzylglycine, N-methylalanine, N-benzylalanine, N-methylphenylalanine, N-benzylphenylalanine, N-methylvaline and N-benzylvaline; and

α-Hydroxy and substituted α-hydroxy amino acids including serine, threonine, allothreonine, phosphoserine and phosphothreonine.

Polypeptides are polymers of amino acids in which a carboxyl group of one amino acid monomer is bonded to an amino or imino group of the next amino acid monomer by an amide bond. Polypeptides include dipeptides, low molecular weight polypeptides (about 1500-5000 MW) and proteins. Proteins optionally contain 3, 5, 10, 50, 75, 100 or more residues, and suitably are substantially sequence-homologous with human, animal, plant or microbial proteins. They include enzymes (e.g., hydrogen peroxidase) as well as immunogens such as KLH, or antibodies or proteins of any type against which one wishes to raise an immune response. The nature and identity of the polypeptide may vary widely.

The polypeptide amidates are useful as immunogens in raising antibodies against either the polypeptide (if it is not immunogenic in the animal to which it is administered) or against the epitopes on the remainder of the compound of this invention.

Antibodies capable of binding to the parental non-peptidyl compound are used to separate the parental compound from mixtures, for example in diagnosis or manufacturing of the parental compound. The conjugates of parental compound and polypeptide generally are more immunogenic than the polypeptides in closely homologous animals, and therefore make the polypeptide more immunogenic for facilitating raising antibodies against it. Accordingly, the polypeptide or protein may not need to be immunogenic in an animal typically used to raise antibodies, e.g., rabbit, mouse, horse, or rat, but the final product conjugate should be immunogenic in at least one of such animals. The polypeptide optionally contains a peptidolytic enzyme cleavage site at the peptide bond between the first and second residues adjacent to the acidic heteroatom. Such cleavage sites are flanked by enzymatic recognition structures, e.g. a particular sequence of residues recognized by a peptidolytic enzyme.

Peptidolytic enzymes for cleaving the polypeptide conjugates of this invention are well known, and in particular include carboxypeptidases. Carboxypeptidases digest polypeptides by removing C-terminal residues, and are specific in many instances for particular C-terminal sequences. Such enzymes and their substrate requirements in general are well known. For example, a dipeptide (having a given pair of residues and a free carboxyl terminus) is covalently bonded through its α-amino group to the phosphorus or carbon atoms of the compounds herein.

Intracellular Targetting

The known experimental or approved HIV integrase inhibitor drugs which can be derivatized in accord with the present invention must contain at least one functional group capable of bonding to the phosphorus atom in the phosphonate moiety. The phosphonate derivatives of Formulas I-XXXIX may cleave in vivo in stages after they have reached the desired site of action, i.e. inside a cell. One mechanism of action inside a cell may entail a first cleavage, e.g. by esterase, to provide a negatively-charged “locked-in” intermediate. Cleavage of a terminal ester grouping in Formulas I-XXXIX thus affords an unstable intermediate which releases a negatively charged “locked in” intermediate.

After passage inside a cell, intracellular enzymatic cleavage or modification of the phosphonate prodrug compound may result in an intracellular accumulation of the cleaved or modified compound by a “trapping” mechanism. The cleaved or modified compound may then be “locked-in” the cell by a significant change in charge, polarity, or other physical property change which decreases the rate at which the cleaved or modified compound can exit the cell, relative to the rate at which it entered as the phosphonate prodrug. Other mechanisms by which a therapeutic effect are achieved may be operative as well. Enzymes which are capable of an enzymatic activation mechanism with the phosphonate prodrug compounds of the invention include, but are not limited to, amidases, esterases, microbial enzymes, phospholipases, cholinesterases, and phosphatases.

In selected instances in which the drug is of the nucleoside type, such as is the case of zidovudine and numerous other antiretroviral agents, it is known that the drug is activated in vivo by phosphorylation. Such activation may occur in the present system by enzymatic conversion of the “locked-in” intermediate with phosphokinase to the active phosphonate diphosphate and/or by phosphorylation of the drug itself after its release from the “locked-in” intermediate as described above. In either case, the original nucleoside-type drug will be convened, via the derivatives of this invention, to the active phosphorylated species.

From the foregoing, it will be apparent that many structurally different known approved and experimental HIV integrase inhibitor drugs can be derivatized in accord with the present invention. Numerous such drugs are specifically mentioned herein. However, it should be understood that the discussion of drug families and their specific members for derivatization according to this invention is not intended to be exhaustive, but merely illustrative.

As another example, when the selected drug contains multiple reactive hydroxyl functions, a mixture of intermediates and final products may again be obtained. In the unusual case in which all hydroxy groups are approximately equally reactive, there is not expected to be a single, predominant product, as each mono-substituted product will be obtained in approximate by equal amounts, while a lesser amount of multiply-substituted product will also result. Generally speaking, however, one of the hydroxyl groups will be more susceptible to substitution than the other(s), e.g. a primary hydroxyl will be more reactive than a secondary hydroxyl, an unhindered hydroxyl will be more reactive than a hindered one. Consequently, the major product will be a mono-substituted one in which the most reactive hydroxyl has been derivatized while other mono-substituted and multiply-substituted products may be obtained as minor products.

Cellular Accumulation Embodiment

Another embodiment of the invention is directed toward compounds capable of accumulating in human PBMC (peripheral blood mononuclear cells). PBMC refer to blood cells having round lymphocytes and monocytes. Physiologically, PBMC are critical components of the mechanism against infection. PBMC may be isolated from heparinized whole blood of normal healthy donors or buffy coats, by standard density gradient centrifugation and harvested from the interface, washed (e.g. phosphate-buffered saline) and stored in freezing medium. PBMC may be cultured in multi-well plates. At various times of culture, supernatant may be either removed for assessment, or cells may be harvested and analyzed (Smith R. et al (2003) Blood 102(7):2532-2540). The compounds of this embodiment may further comprise a phosphonate or phosphonate prodrug. More typically, the phosphonate or phosphonate prodrug has the structure A³ as described herein.

Optionally, the compounds of this embodiment demonstrate improved intracellular half-life of the compounds or intracellular metabolites of the compounds in human PBMC when compared to analogs of the compounds not having the phosphonate or phosphonate prodrug. Typically, the half-life is improved by at least about 50%, more typically at least in the range 50-100%, still more typically at least about 100%, more typically yet greater than about 100%.

In another embodiment, the intracellular half-life of a metabolite of the compound in human PBMCs is improved when compared to an analog of the compound not having the phosphonate or phosphonate prodrug. In such embodiments, the metabolite may be generated intracellularly, e.g. generated within human PBMC. The metabolite may be a product of the cleavage of a phosphonate prodrug within human PBMCs. The phosphonate prodrug may be cleaved to form a metabolite having at least one negative charge at physiological pH. The phosphonate prodrug may be enzymatically cleaved within human PBMC to form a phosphonate having at least one active hydrogen atom of the form P—OH.

Stereoisomers

The compounds of the invention, exemplified by Formula I-XXXIX, may have chiral centers, e.g. chiral carbon, sulfur, or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through well-known techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

The compounds of the invention can also exist as tautomeric isomers in certain cases. All though only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

Salts and Hydrates

The compositions of this invention optionally comprise salts of the compounds herein, especially pharmaceutically acceptable non-toxic salts containing, for example, Na⁺, Li⁺, K⁺, Ca⁺² and Mg⁺². Such salts may include those derived by combination of appropriate cations such as alkali and alkaline earth metal ions or ammonium and quaternary amino ions with an acid anion moiety, typically a carboxylic acid. Monovalent salts are preferred if a water soluble salt is desired.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their un-ionized, as well as zwitterionic form, and combinations with stoichiometric amounts of water as in hydrates.

Also included within the scope of this invention are the salts of the parental compounds with one or more amino acids. Any of the amino acids described above are suitable, especially the naturally-occurring amino acids found as protein components, although the amino acid typically is one bearing a side chain with a basic or acidic group, e.g., lysine, arginine or glutamic acid, or a neutral group such as glycine, serine, threonine, alanine, isoleucine, or leucine.

Methods of Inhibition of HIV Integrase

Another aspect of the invention relates to methods of inhibiting the activity of HIV integrase comprising the step of treating a sample suspected of containing HIV with a composition of the invention.

Compositions of the invention may act as inhibitors of HIV integrase, as intermediates for such inhibitors or have other utilities as described below. The inhibitors will bind to locations on the surface or in a cavity of HIV integrase having a geometry unique to HIV integrase. Compositions binding HIV integrase may bind with varying degrees of reversibility. Those compounds binding substantially irreversibly are ideal candidates for use in this method of the invention. Once labeled, the substantially irreversibly binding compositions are useful as probes for the detection of HIV integrase. Accordingly, the invention relates to methods of detecting HIV integrase in a sample suspected of containing HIV integrase comprising the steps of: treating a sample suspected of containing HIV integrase with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl or amino.

Within the context of the invention, samples suspected of containing HIV integrase include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing an organism which produces HIV integrase, frequently a pathogenic organism such as HIV. Samples can be contained in any medium including water and organic solvent\water mixtures. Samples include living organisms such as humans, and man made materials such as cell cultures.

The treating step of the invention comprises adding the composition of the invention to the sample or it comprises adding a precursor of the composition to the sample. The addition step comprises any method of administration as described above.

If desired, the activity of HIV integrase after application of the composition can be observed by any method including direct and indirect methods of detecting HIV integrase activity. Quantitative, qualitative, and semiquantitative methods of determining HIV integrase activity are all contemplated. Typically one of the screening methods described above are applied, however, any other method such as observation of the physiological properties of a living organism are also applicable.

Organisms that contain HIV integrase include the HIV virus. The compounds of this invention are useful in the treatment or prophylaxis of HIV infections in animals or in man.

However, in screening compounds capable of inhibiting human immunodeficiency viruses, it should be kept in mind that the results of enzyme assays may not correlate with cell culture assays. Thus, a cell based assay should be the primary screening tool.

Screens for HIV Integrase Inhibitors.

Compositions of the invention are screened for inhibitory activity against HIV integrase by any of the conventional techniques for evaluating enzyme activity. Within the context of the invention, typically compositions are first screened for inhibition of HIV integrase in vitro and compositions showing inhibitory activity are then screened for activity in vivo. Compositions having in vitro Ki (inhibitory constants) of less then about 5×10⁻⁶ M, typically less than about 1×10⁻⁷ M and preferably less than about 5×10⁻⁸ M are preferred for in vivo use.

Useful in vitro screens have been described in detail and will not be elaborated here. However, the examples describe suitable in vitro assays.

Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a combination according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of HIV infections as described below.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient.

These veterinary compositions may be administered orally, parenterally or by any other desired route.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

Active ingredients of the invention are also used in combination with other active ingredients. Such combinations are selected based on the condition to be treated, cross-reactivities of ingredients and pharmaco-properties of the combination. For example, when treating viral infections the compositions of the invention are combined with other antivirals such as other protease inhibitors, nucleoside reverse transcriptase inhibitors, non-nucleoside reverse transcriptase inhibitors or HIV integrase inhibitors.

Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

Combination Therapy

It is also possible to combine any compound of the invention with one or more other active ingredients in a unitary dosage form for simultaneous or sequential administration to an HIV infected patient. The combination therapy may be administered as a simultaneous or sequential regimen. When administered sequentially, the combination may be administered in two or more administrations. Second and third active ingredients in the combination may have anti-HIV activity. Exemplary active ingredients to be administered in combination with compounds of the invention are protease inhibitors, nucleoside reverse transcriptase inhibitors, non-nucleoside reverse transcriptase inhibitors, and HIV integrase inhibitors.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

In another embodiment the invention provides an HIV integrase inhibitor compound provided that the compound is not 4-(3-benzyl-phenyl)-2-hydroxy-4-oxo-but-2-enoic acid, 1-[5-(4-fluoro-benzyl)-furan-2-yl]-3-hydroxy-3-(1H-[1,2,4]triazol-3-yl)-propenone, or 5-(1,1-dioxo-116-[1,2]thiazinan-2-yl)-8-hydroxy-quinoline-7-carboxylic acid 4-fluoro-benzylamide.

In another embodiment the invention provides an HIV integrase inhibitor compound provided that the compound is not: 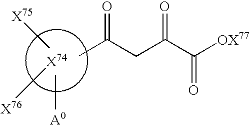
wherein X⁷⁴ (—X⁷⁵, —X⁷⁶) is not phenyl substituted with benzyl and X⁷⁷ is not hydrogen; or the compound is not: 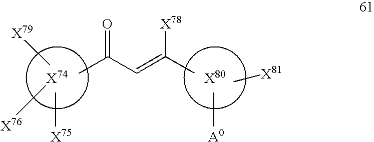
wherein X⁷⁴ (—X⁷⁵, X⁷⁶, —X⁷⁹) is not furan substituted with p-fluorobenzyl, when X⁷⁸ is hydroxy, and X⁸⁰ (—X⁸¹) is 1H-[1,2,4]triazole.

It is also possible to combine a compound of the invention with a second or third active ingredient in a unitary dosage form for simultaneous or sequential administration. When administered sequentially, the combination may be administered in two or three administrations. The second or third active ingredient may have anti-HIV activity and include protease inhibitors (PI), nucleoside reverse transcriptase inhibitors (NRTI), non-nucleoside reverse transcriptase inhibitors (NNRTI), and integrase inhibitors. Exemplary second or third active ingredients to be administered in combination with a compound of the invention are shown in Table C.

Table C

5,6 dihydro-5-azacytidine
5-aza 2′deoxycytidine
5-azacytidine
5-yl-carbocyclic 2′-deoxyguanosine (BMS200,475)
9 (arabinofuranosyl)guanine; 9-(2′ deoxyribofuranosyl)guanine
9-(2′-deoxy 2′fluororibofuranosyl)-2,6-diaminopurine
9-(2′-deoxy 2′fluororibofuranosyl)guanine
9-(2′-deoxyribofuranosyl)-2,6 diaminopurine
9-(arabinofuranosyl)-2,6 diaminopurine
Abacavir, Ziagen®
Acyclovir, ACV; 9-(2-hydroxyethoxylmethyl)guanine
Adefovir dipivoxil, Hepsera®
amdoxivir, DAPD
Amprenavir, Agenerase®
araA; 9-b-D-arabinofuranosyladenine (Vidarabine)
atazanivir sulfate (Reyataz®)
AZT; 3′-azido-2′,3′-dideoxythymdine, Zidovudine, (Retrovir®)
BHCG; (.+−.)-(1a,2b,3a)-9-[2,3-bis(hydroxymethyl)cyclobutyl]guanine
BMS200,475; 5-yl-carbocyclic 2′-deoxyguanosine
Buciclovir; (R) 9-(3,4-dihydroxybutyl)guanine
BvaraU; 1-b-D-arabinofuranosyl-E-5-(2-bromovinyl)uracil (Sorivudine)
Calanolide A
Capravirine
CDG; carbocyclic 2′-deoxyguanosine
Cidofovir, HPMPC; (S)-9-(3-hydroxy-2-phosphonylmethoxypropyl)cytosine
Clevudine, L-FMAU; 2′-Fluoro-5-methyl-β-L-arabino-furanosyluracil
Combivir® (lamivudine/zidovudine)
Cytallene; [1-(4′-hydroxy-1′,2′-butadienyl)cytosine]
d4C; 3′-deoxy-2′,3′-didehydrocytidine
DAPD; (−)-β-D-2,6-diaminopurine dioxolane
ddA; 2′,3′-dideoxyadenosine
ddAPR; 2,6-diaminopurine-2′,3′-dideoxyriboside
ddC; 2′,3′-dideoxycytidine (Zalcitabine)
ddI; 2′,3′-dideoxyinosine, didanosine, (Videx®, Videx® EC)
Delavirdine, Rescriptor®
Didanosine, ddI, Videx®; 2′,3′-dideoxyinosine
DXG; dioxolane guanosine
E-5-(2-bromovinyl)-2′-deoxyuridine
Efavirenz, Sustiva®
Emtricitabine; (−)-cis FTC (Emtriva™)
Enfuvirtide (Fuzeon®)
F-ara-A; fluoroarabinosyladenosine (Fludarabine)
FDOC; (−)-β-D-5-fluoro-1-[2-(hydroxymethyl)-1,3-dioxolane]cytosine
FEAU; 2′-deoxy-2′-fluoro-1-β-D-arabinofuranosyl-5-ethyluracil
FIAC; 1-(2-deoxy-2-fluoro-β-D-arabinofuranosyl)-5-iodocytosine
FIAU; 1-(2-deoxy-2-fluoro-β-D-arabinofuranosyl)-5-iodouridine
FLG; 2′,3′-dideoxy-3′-fluoroguanosine
FLT; 3′-deoxy-3′-fluorothymidine
Fludarabine; F-ara-A; fluoroarabinosyladenosine
FMAU; 2′-Fluoro-5-methyl-b-L-arabino-furanosyluracil
FMdC
Foscarnet; phosphonoformic acid, PFA
FPMPA; 9-(3-fluoro-2-phosphonylmethoxypropyl)adenine
Gancyclovir, GCV; 9-(1,3-dihydroxy-2-propoxymethyl)guanine
GS-7340; 9-[R-2-[[(S)-[[(S)-1-(isopropoxycarbonyl)ethyl]amino]-phenoxyphosphinyl]methoxy]propyl] adenine
HPMPA; (S)-9-(3-hydroxy-2-phosphonylmethoxypropyl)adenine
HPMPC; (S)-9-(3-hydroxy-2-phosphonylmethoxypropyl)cytosine (Cidofovir)
Hydroxyurea, Droxia®
Indinavir, Crixivan®
Kaletra® (lopinavir/ritonavir)
Lamivudine, 3TC, Epivir™; (2R,5S,cis)-4-amino-1-(2-hydroxymethyl-1,3-oxathiolan-5-yl)-(1H)-pyrimidin-2-one
L-d4C; L-3′-deoxy-2′,3′-didehydrocytidine
L-ddC; L-2′,3′-dideoxycytidine
L-Fd4C; L-3′-deoxy-2′,3′-didehydro-5-fluorocytidine
L-FddC; L-2′,3′-dideoxy-5-fluorocytidine
Lopinavir
Nelfinavir, Viracept®
Nevirapine, Viramune®
Oxetanocin A; 9-(2-deoxy-2-hydroxymethyl-beta-D-erythro-oxetanosyl)adenine
Oxetanocin G; 9-(2-deoxy-2-hydroxymethyl-β-D-erythro-oxetanosyl)guanine
Penciclovir
PMEDAP; 9-(2-phosphonylmethoxyethyl)-2,6-diaminopurine
PMPA, tenofovir; (R)-9-(2-phosphonylmethoxypropyl)adenine
PPA; phosphonoacetic acid
Ribavirin; 1-β-D-ribofuranosyl-1,2,4-triazole-3-carboxamide
Ritonavir, Norvir®
Saquinavir, Invirase®, Fortovase®
Sorivudine, BvaraU; 1-β-D-arabinofuranosyl-E-5-(2-bromovinyl)uracil
Stavudine, d4T, Zerit®; 2′,3′-didehydro-3′-deoxythymidine
Tenofovir disoproxil fumarate (TDF, Viread®)
Trifluorothymidine, TFT; Trifluorothymidine
Trizivir® (abacavir sulfate/lamivudine/zidovudine)
Vidarabine, araA; 9-β-D-arabinofuranosyladenine
Zalcitabine, Hivid®, ddC; 2′,3′-dideoxycytidine
Zidovudine, AZT, Retrovir®; 3′-azido-2′,3′-dideoxythymdine
Zonavir; 5-propynyl-1-arabinosyluracil
   Metabolites of the Compounds of the Invention

Also falling within the scope of this invention are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, the invention includes novel and unobvious compounds produced by a process comprising contacting a compound of this invention with a mammal for a period of time sufficient to yield a metabolic product thereof. Such products typically are identified by preparing a radiolabelled (e.g. C¹⁴ or H³) compound of the invention, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no HIV integrase inhibitory activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The phosphonate prodrugs of the invention typically will be largely stable in the digestive system but substantially hydrolyzed to the parental drug in the digestive lumen, liver or other metabolic organ, or within cells in general.

Exemplary Methods of Making the Compounds of the Invention.

General aspects of exemplary methods are described below and in the Examples for the making, i.e. preparation, synthesis, of Formula I-XXXIX compounds of the invention. Each of the products of the following processes is optionally separated, isolated, and/or purified prior to its use in subsequent processes.

The compounds of the invention may be prepared by a variety of synthetic routes and methods known to those skilled in the art. The invention also relates to methods of making the compounds of the invention. The compounds are prepared by any of the applicable techniques of organic synthesis. Many such techniques are well known in the art. However, many of the known techniques are elaborated in: Compendium of Organic Synthetic Methods, John Wiley & Sons, New York, Vol. 1, Ian T. Harrison and Shuyen Harrison, 1971; Vol. 2, Ian T. Harrison and Shuyen Harrison, (1974); Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., Advanced Organic Chemistry, Third Edition, John Wiley & Sons, New York, 1985; Comprehensive Organic Synthesis, Selectivity Strategy & Efficiency in Modern Organic Chemistry (9 Volume set) Barry M. Trost, Editor-in-Chief, Pergamon Press, New York, 1993.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g. inert gas environments) are common in the art and will be applied when applicable.

The terms “treated”, “treating”, “treatment”, and the like, mean contacting, mixing, reacting, allowing to react, bringing into contact, and other terms common in the art for indicating that one or more chemical entities is treated in such a manner as to convert it to one or more other chemical entities. This means that “treating compound one with compound two” is synonymous with “allowing compound one to react with compound two”, “contacting compound one with compound two”, “reacting compound one with compound two”, and other expressions common in the art of organic synthesis for reasonably indicating that compound one was “treated”, “reacted”, “allowed to react”, etc., with compound two.

“Treating” indicates the reasonable and usual manner in which organic chemicals are allowed to react. Normal concentrations (0.01M to 10M, typically 0.1M to 1M), temperatures (−100° C. to 250° C., typically −78° C. to 150° C., more typically −78° C. to 100° C., still more typically 0° C. to 100° C.), reaction vessels (typically glass, plastic, metal), solvents, pressures, atmospheres (typically air for oxygen and water insensitive reactions or nitrogen or argon for oxygen or water sensitive), etc., are intended unless otherwise indicated. The knowledge of similar reactions known in the art of organic synthesis are used in selecting the conditions and apparatus for “treating” in a given process. In particular, one of ordinary skill in the art of organic synthesis selects conditions and apparatus reasonably expected to successfully carry out the chemical reactions of the described processes based on the knowledge in the art.

Modifications of each of the exemplary schemes above and in the examples (hereafter “exemplary schemes”) leads to various analogs of the specific exemplary materials produce. The above cited citations describing suitable methods of organic synthesis are applicable to such modifications.

In each of the exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

A single stereoisomer, e.g. an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller, C. H., (1975) J. Chromatogr., 113:(3) 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Drug Stereochemistry, Analytical Methods and Pharmacology, Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g. (−) menthyl chloroformate in the presence of base, or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (Chiral Liquid Chromatography (1989) W. J. Lough, Ed. Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

All literature and patent citations above are hereby expressly incorporated by reference at the locations of their citation. Specifically cited sections or pages of the above cited works are incorporated by reference with specificity. The invention has been described in detail sufficient to allow one of ordinary skill in the art to make and use the subject matter of the following Embodiments. It is apparent that certain modifications of the methods and compositions of the following Embodiments can be made within the scope and spirit of the invention.

A number of exemplary methods for the preparation of the compounds, Formulas I-XXXIX, of the invention are provided herein. These methods are intended to illustrate the nature of such preparations and are not intended to limit the scope of applicable methods.

Deliberate use may be made of protecting groups to mask reactive functionality and direct reactions regioselectively (Greene, et al (1991) “Protective Groups in Organic Synthesis”, 2nd Ed., John Wiley & Sons). For example, useful protecting groups for the 8-hydroxyl group and other hydroxyl substituents include methyl, MOM (methoxymethyl), trialkylsilyl, benzyl, benzoyl, trityl, and tetrahydropyranyl. Certain aryl positions may be blocked from substitution, such as the 2-position as fluorine.

Formula I Compounds

Exemplary methods of synthesis of Formula I compounds are described below in Schemes 1-10 and 15-17. One method of synthesis of Formula I compounds of the invention is cyclization of a succinimide compound with a pyridine dicarboxylate compound to give tricyclic compounds (Murray and Semple, Synthesis (1996) 11:80-82; Jones and Jones, Jour. Chem. Soc., Perkin Transactions I (1973) 26-32), according to Scheme 1.

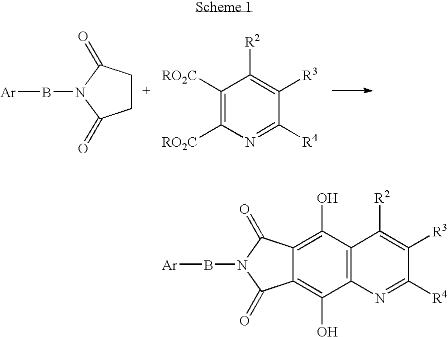

Alternatively, a succinimide with a labile protecting group (P) on the nitrogen may be reacted with a pyridine dicarboxylate compound. P may be an acid-labile protecting group, such as trialkylsilyl. Trialkylsilyl groups may also be removed with fluoride reagents. After P is removed, a variety of Ar-L groups may be covalently attached, according to Scheme 2.

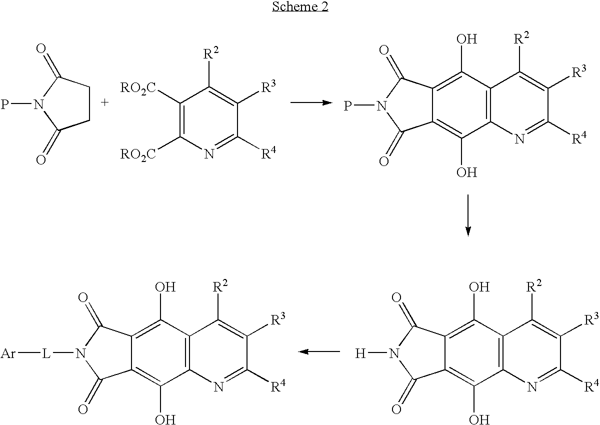

Imide compounds can be reduced with dissolving metal reducing agents, e.g. Zn, or hydride reagents, e.g. NaBH₄, to form a lactam. Exemplary regioselective conversions shown in Scheme 3 include:

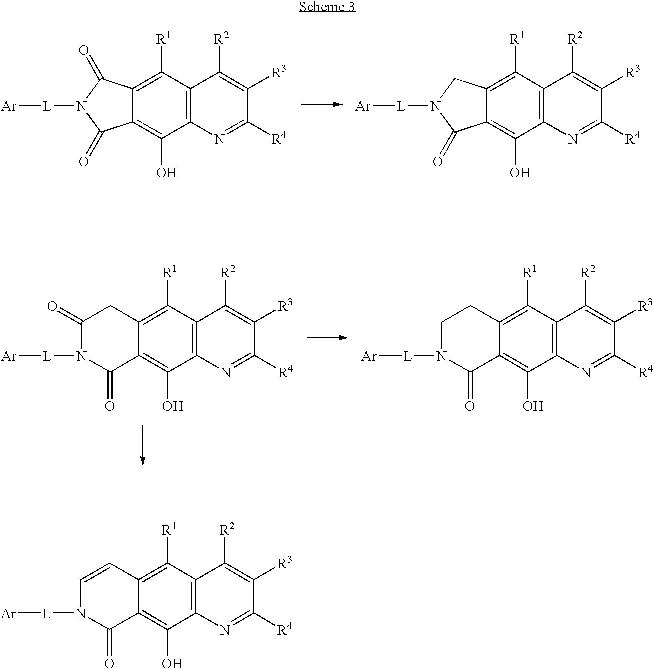

Imide compounds may also be reduced to the hydroxylactam under mild conditions. Reductions with sodium borohydride and cerium or samarium salts have been shown to proceed with regioselectivity on asymmetric imides (Mase, et al J. Chem. Soc. Perkin Communication 1 (2002) 707-709), as in Scheme 4, upper. Grignard reagents and acetylenic anions (Chihab-Eddine, et al Tetrahedron Lett. (2001) 42:573-576) may also add with regioselectivity to an imide carbonyl to form alkyl-hydroxylactam compounds, as in Scheme 4, lower). The phenolic oxygen groups may be protected and deprotected as necessary to furnish yield reactions.

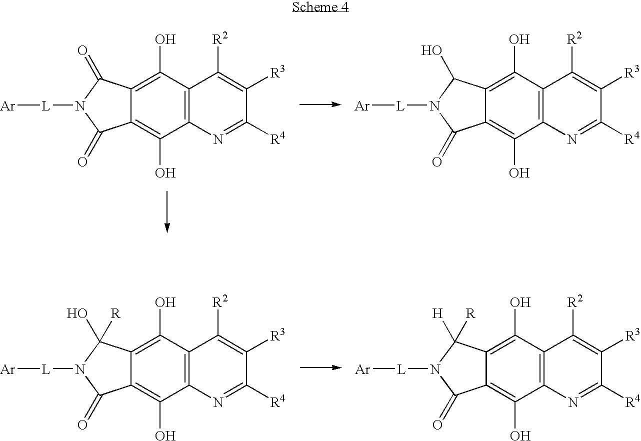

Another synthetic route to the compounds of the invention proceeds through substituted quinoline intermediates (Clemence, et al U.S. Pat. No. 5,324,839; Billhardt-Troughton, et al U.S. Pat. No. 5,602,146; Matsumura, J. Amer. Chem. Soc. (1935) 57:124-128) having the general formula: 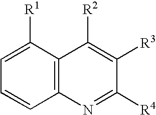
5,8-Dihydroxy quinoline compounds may be elaborated according to Scheme 5:

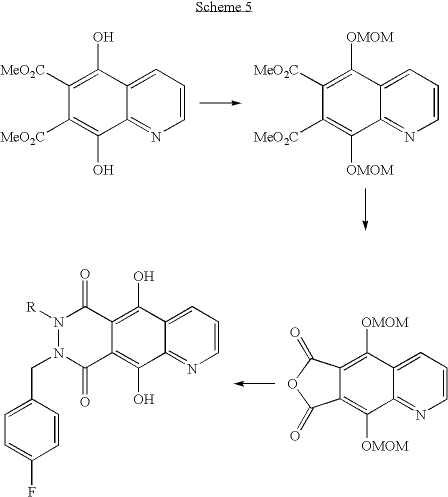

The cyclic anhydride below may be regioselectively esterified to give the compounds of the invention, for example via the route in Scheme 6

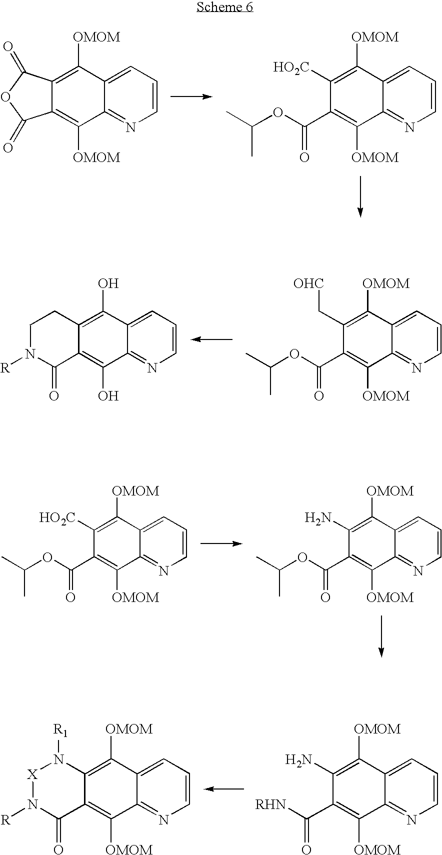

where MOM is methoxymethyl and X is, for example, C(═O), CRC(═O), C(═O)C(═O), and 502. See Ornstein, et al Jour. Med. Chem. (1989) 32:827-833. The same chemistry can be applied to the 5-membered lactam synthesis to control the regiochemistry as in Scheme 7:

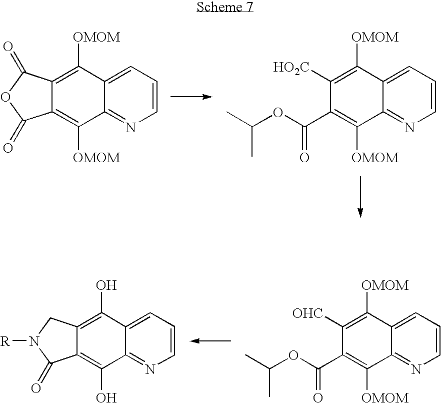

A cyclic imide may be conveniently alkylated, acylated, or otherwise reacted to form a broad array of compounds with Ar-L groups:

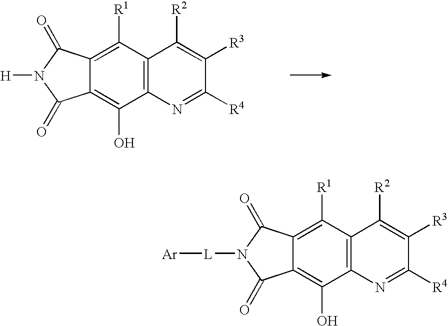

The Ar-L group may be attached as one reactant group, for example as an alkylating reagent like benzyl bromide (Ar=phenyl, L=CH₂) or a sulfonating reagent, like 4-methoxyphenyl sulfonyl chloride (Ar=4-methoxyphenyl, L=S(═O)₂. Alternatively, the Ar-L group may be attached by a multi step process. For example, the imide nitrogen may react with a sulfurizing reagent such as 2,2-dipyridyl disulfide to form an N-sulfide intermediate (Ar=2-pyridyl, L=S). Such an intermediate may be further elaborated to a variety of Ar-L groups where L is S, S(═O) or S(═O)₂.

Another synthetic route to the compounds of the invention proceeds through 7-substituted, 8-quinolinol intermediates (Zhuang, et al WO 02/36734; Vaillancourt, et al U.S. Pat. No. 6,310,211; Hodel, U.S. Pat. No. 3,113,135) having the general formulas, including aryl substituted compounds:

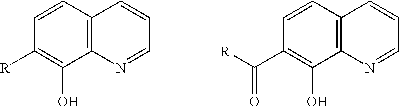

Annulation of the third, 5-7 membered ring can be conducted by appropriate selection of aryl substituents on the quinoline ring system, utilizing known synthetic transformations to give compounds of Formula I. For example, methods for coupling carboxylic acids and other activated acyl groups with amines to form carboxamides are well known in the art (March, J. Advanced Organic Chemistry, 3rd Edition, John Wiley & Sons, 1985, pp. 370-376). An exemplary cyclization includes the following:

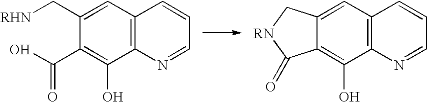

Scheme 8 below shows another synthetic route to compounds of the invention, i.e. Formula I. This route proceeds by cyclization of a 2-O-protected, 3 halo-aniline compound with an (α,β-unsaturated carbonyl compound to give a functionalized quinoline. The α,β-unsaturated carbonyl compound may be, for example, an aldehyde (X=H), ketone (X=R), ester (X=OR), amide (X=NR₂), acyl halide (X=Cl), or anhydride. Carbonylation via palladium catalysis can give an ester which may be elaborated to the amide functionality and cyclization to form a 5, 6, or 7 membered ring. The R group of phenolic oxygen may be a labile protecting group, e.g. trialkylsilyl or tetrahydropyranyl, which may be removed at a step in the synthetic route, or it may be a substituent which is retained in the putative integrase inhibitor compound.

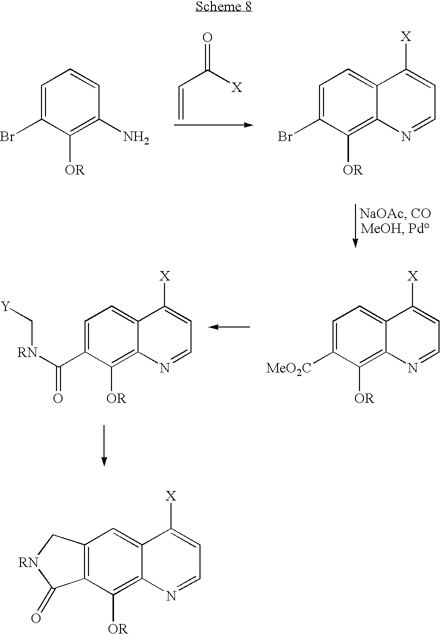

Halo quinoline intermediates may undergo a flexible array of nucleophilic aromatic substitutions and Suzuki-type reactions, as shown in Scheme 9 below. Suzuki coupling of aryl halide compounds with acetylenic and vinylic palladium complexes are carbon-carbon bond forming reactions under relatively mild conditions. In some instances it may be necessary to block the 2 position to direct reaction at the desired aryl position.

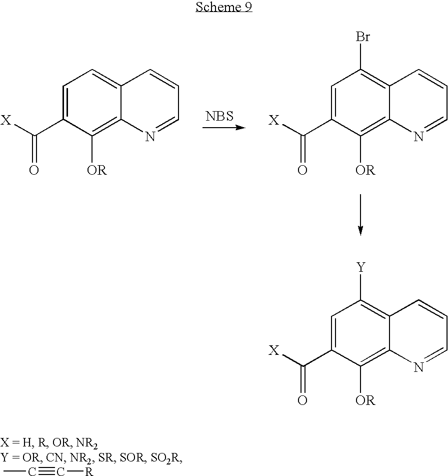

Formula I compounds with a 5,9-dihydroxy-pyrrolo[3,4-g]quinoline-6,8-dione were prepared by selective protection of the C9 phenol in 5,9-dihydroxy-pyrrolo[3,4-g]quinoline-6,8-dione. The C9 phenol was protected with a TIPS group and the C5 phenol could then be alkylated or acylated (Scheme 10). 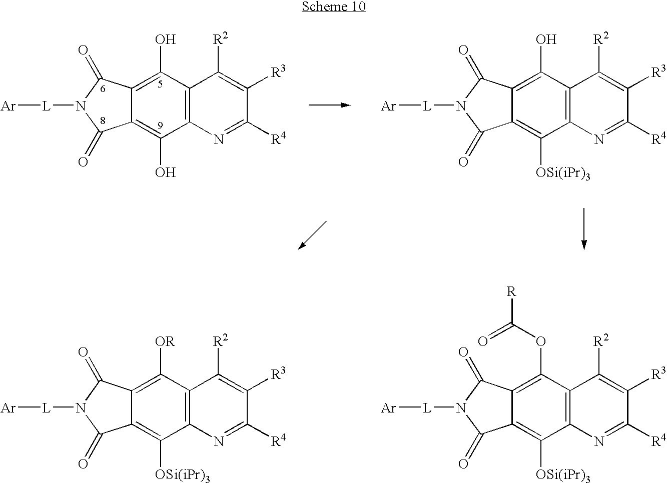
Formula III Compounds

Formula III compounds may be prepared by the following methods in Schemes 11-14:

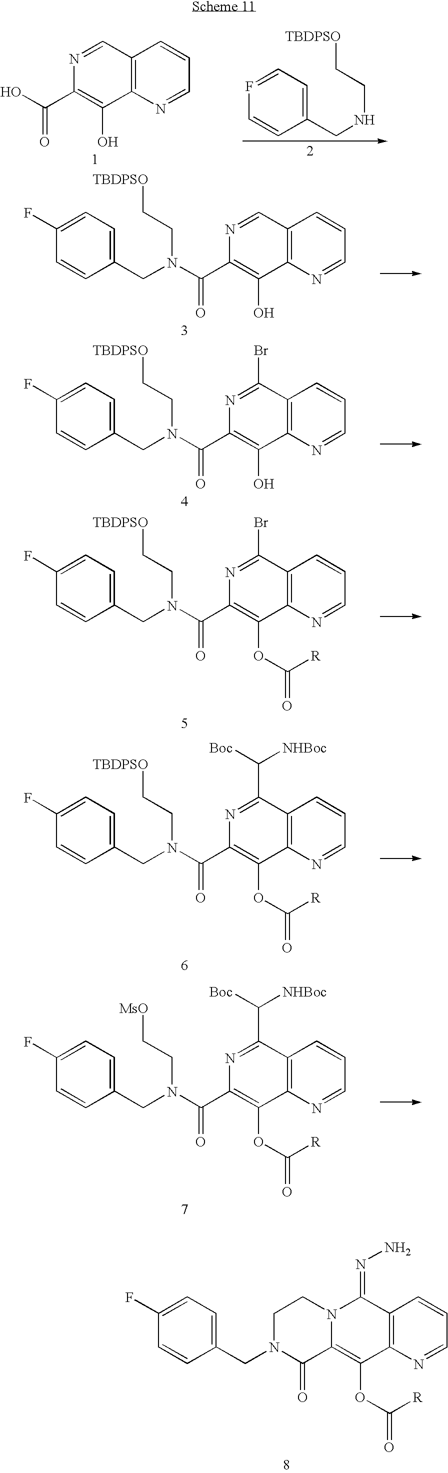

The acid 1 (WO02/30930, p. 173) may be reacted with amine 2 (prepared according to the methods described by T. Morie, et al, Chem. Pharm. Bull., 42, 1994, 877-882; D. Wenninger, et al, Nucleosides Nucleotides, 16, 1997, 977-982) by the method of peptide coupling such as described in WO02/30930, p. 173 to form amide 3. Bromination with NBS generates compound 4. The phenol is protected with a bulky acyl group such as pivaloyl. Displacement of bromine at C5 of naphthyridine by Bis-boc protected hydrazine is achieved using the method reported by J. B. Arteburn, et al, Org. Lett., 3, 2001, 1351-1354. The silyl protecting group is removed by TBAF (T. Green and P. Wuts, “Protective Groups in Organic Synthesis”, p. 142, Wiley Science, 1999) and mesylate 7 is formed by reacting the alcohol formed with methanesulfonyl chloride. Treatment of compound 7 with TFA followed by heating hydrazino mesylate in the basic condition affords hydrazono triaza anthracene 8.

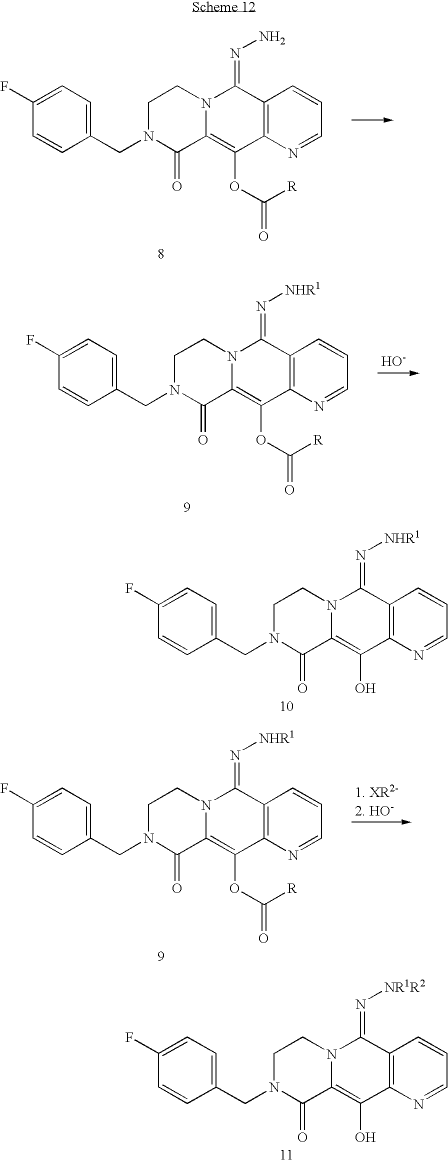

Compound 8 is converted to many different derivatives, e.g. carbazones 9 (R¹=COR³) are generated by reaction with acid chlorides or activated carboxylic acids. Carbamates 9 (R¹=COOR³) are obtained upon reaction of 8 with chloro formates ClCOOR³. Semicarbazones 9 (R¹=CONR²R³) are formed using isocyanates or N,N-dialkyl chloroformaides. Thiosemicarbazones 9 (R¹=CSNR³R⁴) are generated with thioisocyanates. Sulfonyl ureas 9 (R¹=SO₂NR³R⁴) are obtained by reaction of 8 with sulfamoyl chlorides using procedures reported by M. L. Matier, et al, J. Med. Chem., 15, 1972, 538-541. The simple sulfonamides are produced when 8 reacts with sulfonyl chlorides. The ester group in compounds 9 is removed upon saponification to give compound 10.

Alternatively, many of hydrazone derivatives 9 are subjected to alkylation followed by saponification to afford compounds 11.

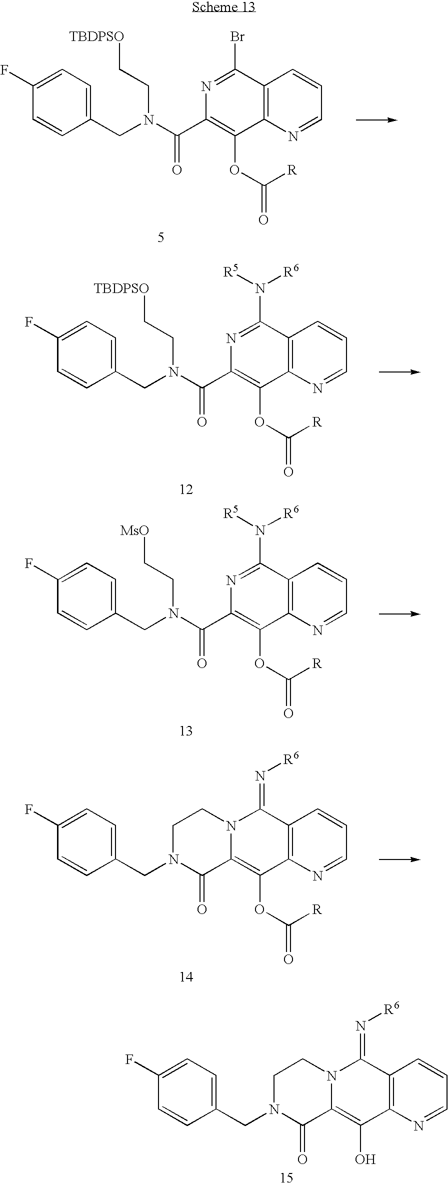

Compound 5 from Scheme 11 is reacted with a substituted hydroxyamine or amine (R⁵=Boc; R⁶=OR^{a} or alkyl) in a manner similar to that described by L. A., Carpino et al, Org. Lett., 3, 2001, 2793-2795 to give derivative 12. After transforming the silyl protected hydroxyl in 12 to a leaving group such as the mesylate in 13, cyclization is accomplished in the heating condition and the presence of a base to afford compound 14. Final deprotection by hydrolysis of 14 gives compound 15.

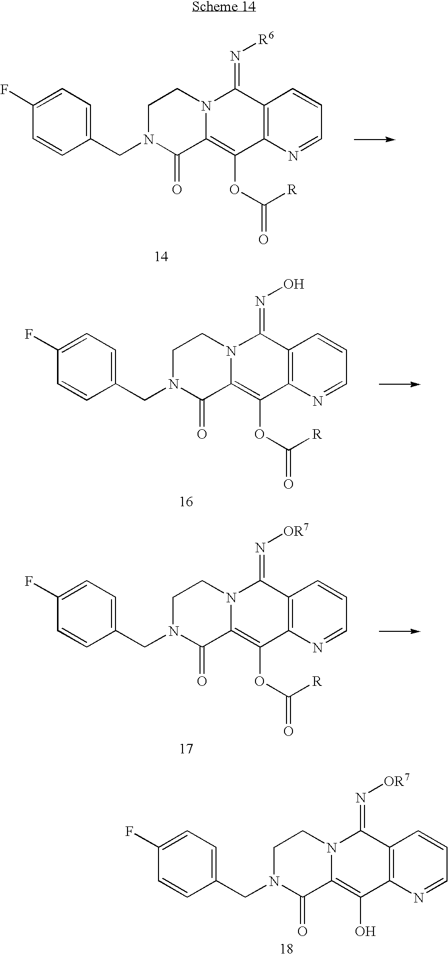

When R⁶ in 14 is OR^{a}, or where R^{a} can be removed, oxime 16 is obtained and can be functionalized with many reagents to yield compound 17. Hydrolysis of ester group affords 18. For example, when 16 is treated with an alkyl halide (R⁷—X) or an alcohol under Mitsunobu condition, an ether 18 is formed. When an isocyanate or thioisocyanate is applied, a carbamate or thiocarbamate 18 (R⁷: C(═O)NHR⁸ or C(═S)NHR⁸) is generated. An N,N-disubstitued carbamate 18 (R⁷: C(═O)NR²R³) is obtained when a chloroformate ClC(═O)NR²R³ is reacted with 16. Similarly, treating 16 with a sulfamoyl chlorides affords a sulfamate 18 (R⁷: SO₂NR¹R²).

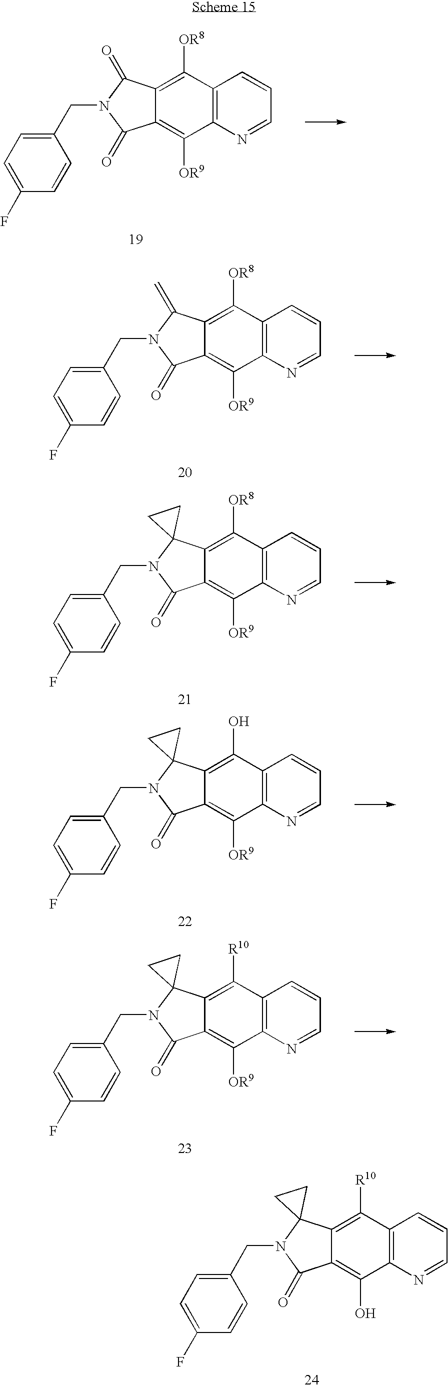

Scheme 15 depicts one of the methods to prepare a spiro-cyclopropane-containing lactam fused to quinoline, an embodiment of Formula I. A differentially protected phenol 19 is used where R⁸ can be a removable ether group such as trimethylsilyethyl ether and R⁹ can be a bulky group such as diphenylmethyl or t-butyl ether. The carbonyl of C6 is converted to an olefin regioselectively by treating 19 with methylmagnesium bromide followed by dehydration of aminal to give 20. Carbene insertion by Simmons-Smith reaction (for example, Y. Biggs et al, JOC, 57, 1992, 5568-5573) produces cyclopropane 21. Selective removal of R⁸ by TBAF followed by fictionalization using the methods described in many examples leads to compound 24.

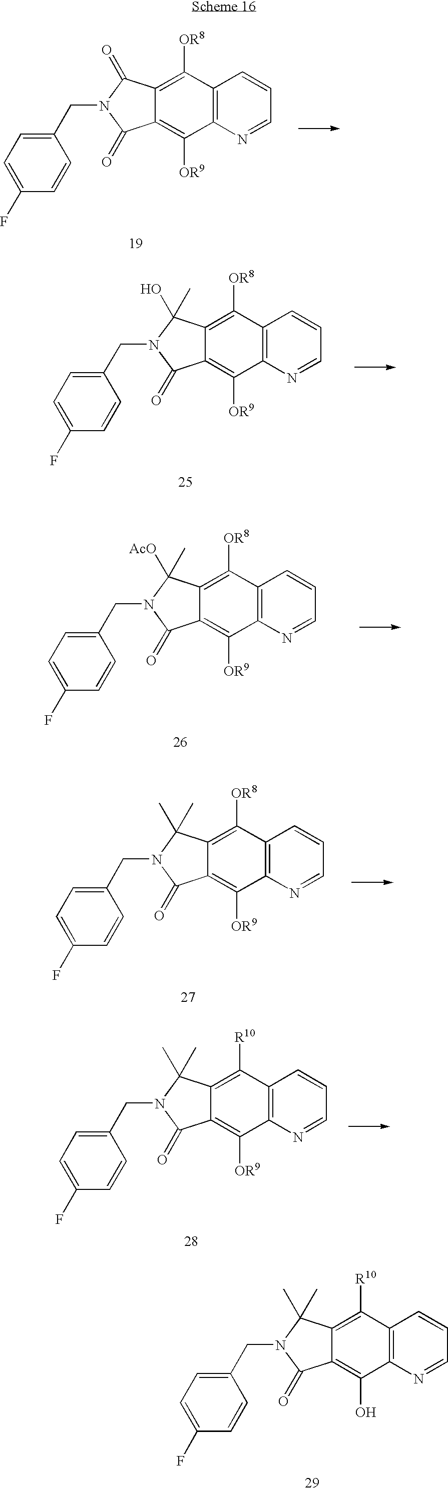

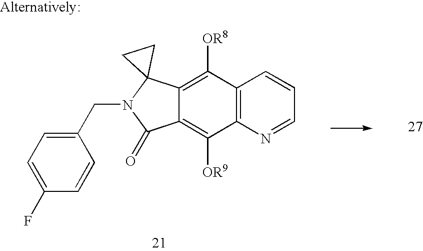

A dimethyl substituted lactam can be prepared by reacting 19 with a Grignard reagent followed by converting aminal 25 to acetate 26 and treating 26 with Me₃Al/TMSOTf, a method reported by C. U. Kim, et al, Tetrahedron Letters, 35, 1994, 3017-3020, to afford 27. An alternative method can be used by reducing cyclopropane 21 with PtO₂/H₂ as reported by C. K. Cheung et al, JOC, 54, 1989, 570-573, to give 27.

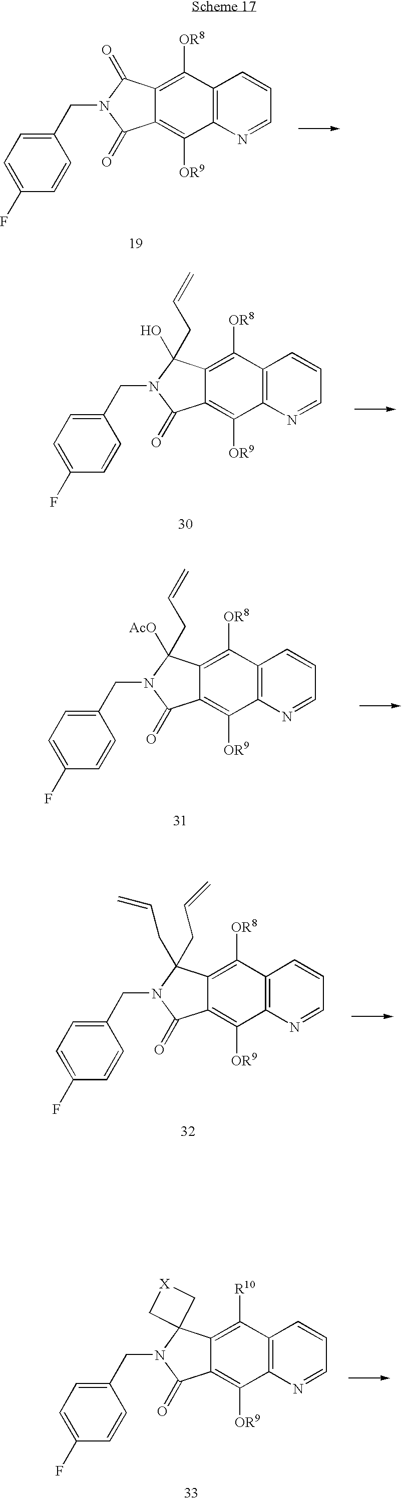

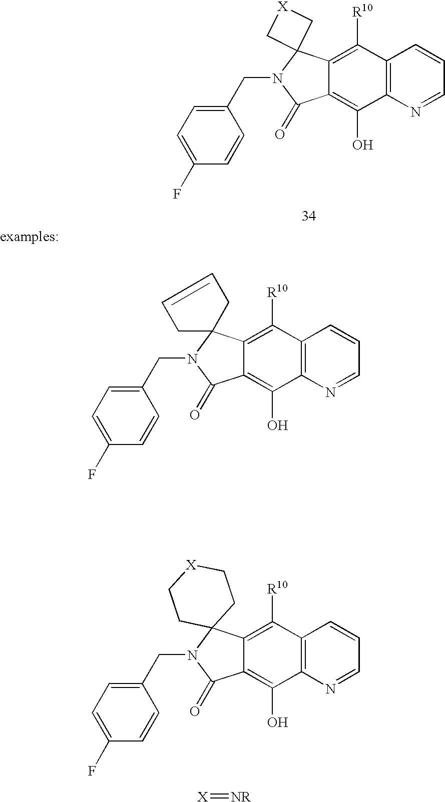

Another version of modified lactam can be obtained according to Scheme 17. Treating 19 with an allyl Grignard reagent gives 30. Activating aminal 30 by forming acetate 31 followed by treating 31 with allyl trimethylsilane mediated by a Lewis acid such as TMSOTf affords 32. Cyclization can be achieved by using Grubb's RCM (ring closure metathesis) method (P. Schwab et al, Angew. Chem. Intl. 34, 1995, 2039). Alternatively, the terminal olefins in 32 can be converted to aldehydes and reductive amination leads to a spiro-piperidine. 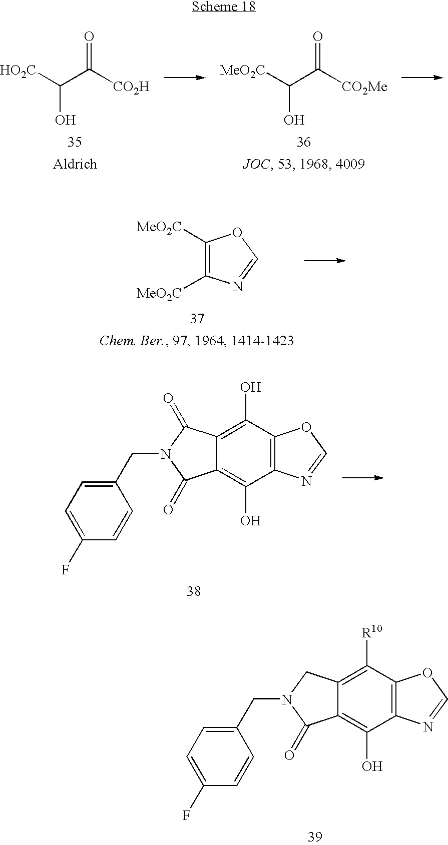 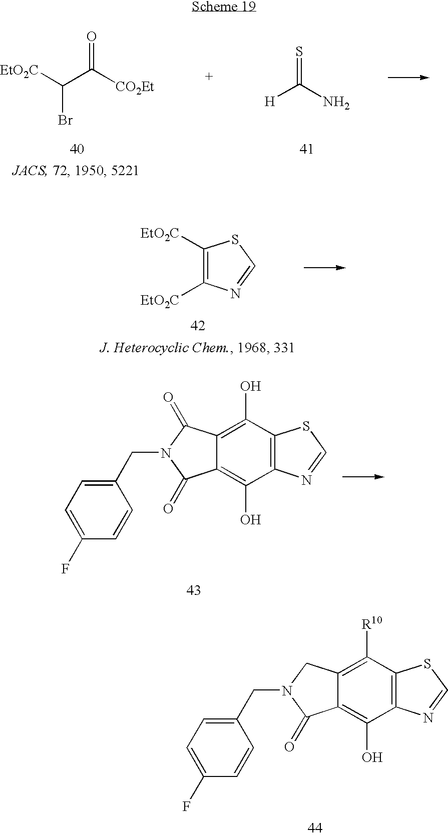 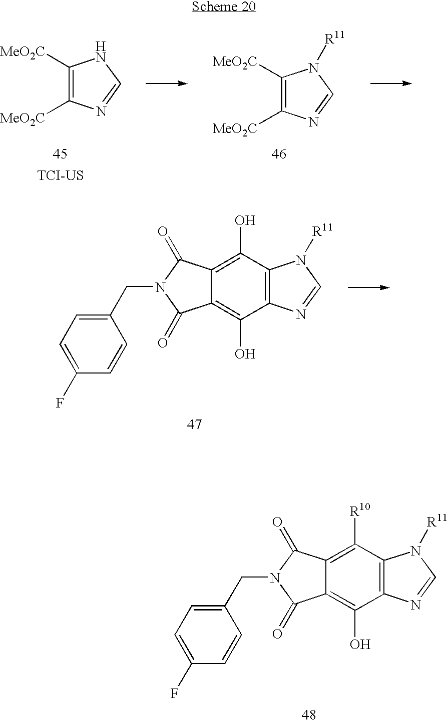 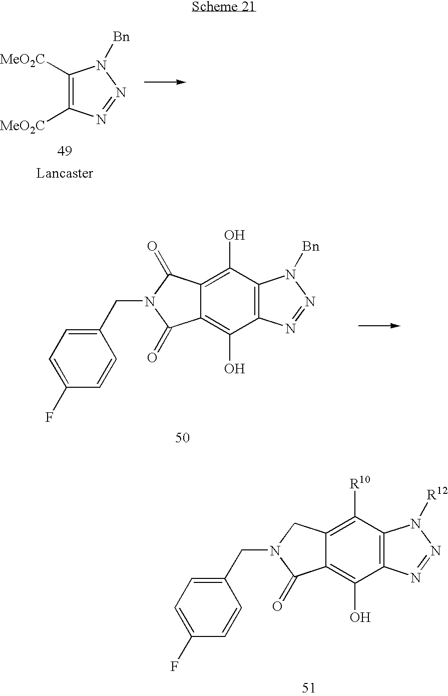 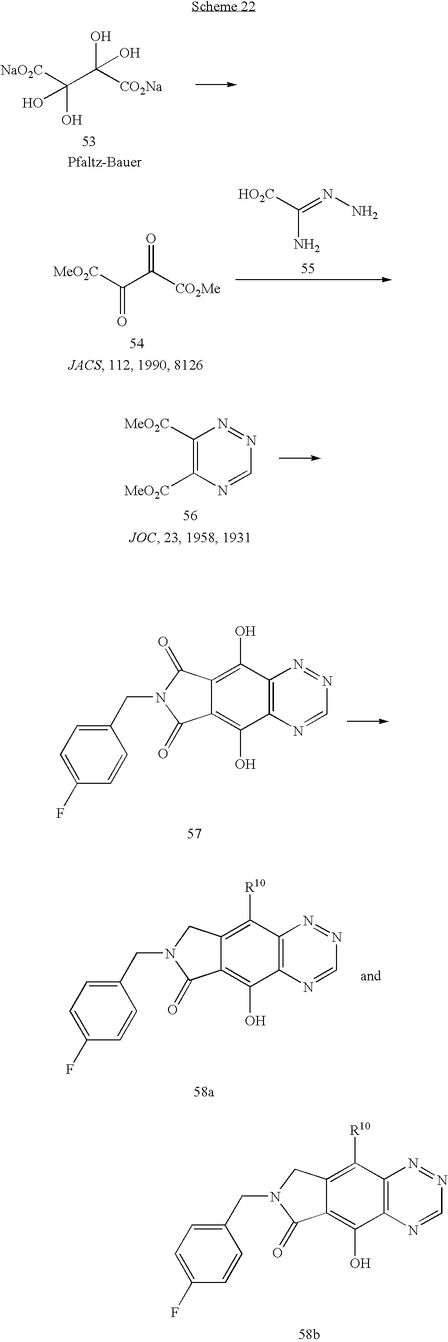 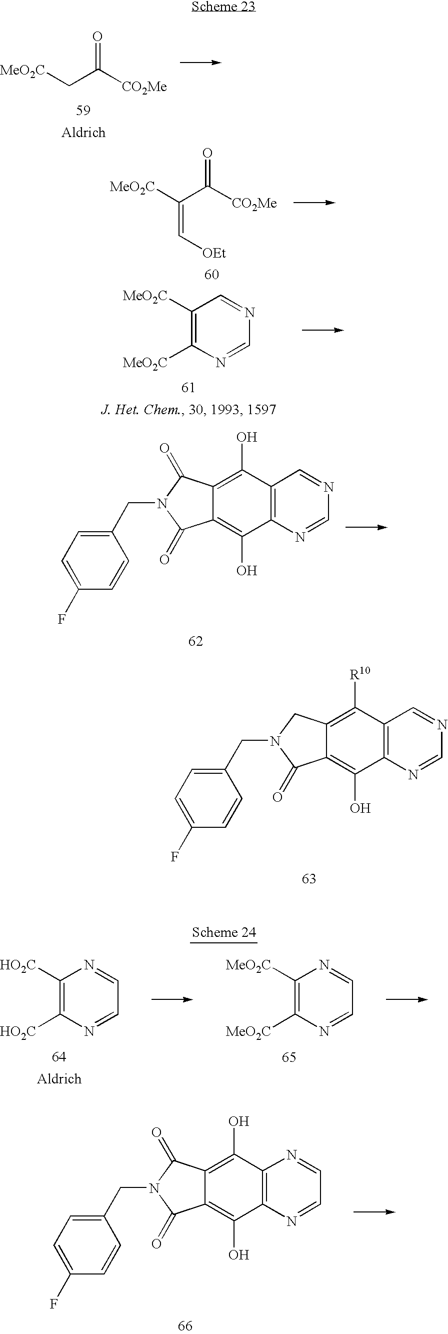 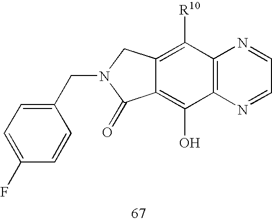
Many tricyclic compounds can bear a heterocycle different from 9-hydroxy-6,7-dihydro-pyrrolo[3,4-g]quinolin-8-one, i.e. Formula IV. Some examples and methods to prepare Formula IV compounds are depicted in Schemes 18-24 above.
Preparation of the Intermediate Phosphonate Esters Iaa-IVcc.

The structures of the intermediate phosphonate esters Iaa to IVcc are shown in Chart 1, in which the substituents R¹, R², R³, R⁴, A¹ and A² are as previously defined. The groups A^{1a} and A^{2a} are the same as the groups A¹ and A², except that a substituent link —P(O)(OR⁵)₂ is appended. The substituent R⁵ is hydrogen, alkyl, alkenyl, aralkyl, or aryl. Subsequent chemical modifications to the compounds Iaa to Vcc, as described herein, permit the synthesis of the final compounds of this invention.

The intermediate compounds Iaa to IVcc incorporate a phosphonate moiety (R⁵O)₂P(O) connected to the nucleus by means of a variable linking group, designated as “link” in the attached structures. Chart 2 illustrates examples of the linking groups present in the structures Iaa-IVcc.

Schemes A1-A33 illustrate the syntheses of the intermediate phosphonate compounds of this invention, Iaa-IVcc, and of the intermediate compounds necessary for their synthesis.

The methods described for the introduction of phosphonate substituents are, with modifications made by one skilled in the art, transferable within the substrates I-V. For example, reaction sequences which produce the phosphonates Iaa are, with appropriate modifications, applicable to the preparation of the phosphonates IIaa, IIIaa, or IVaa. Methods described below for the attachment of phosphonate groups to reactive substituents such as OH, NH₂, CH₂Br, COOH, CHO etc are applicable to each of the scaffolds I-V.

Scheme A34 illustrates methods for the interconversion of phosphonate diesters, monoesters and acids. 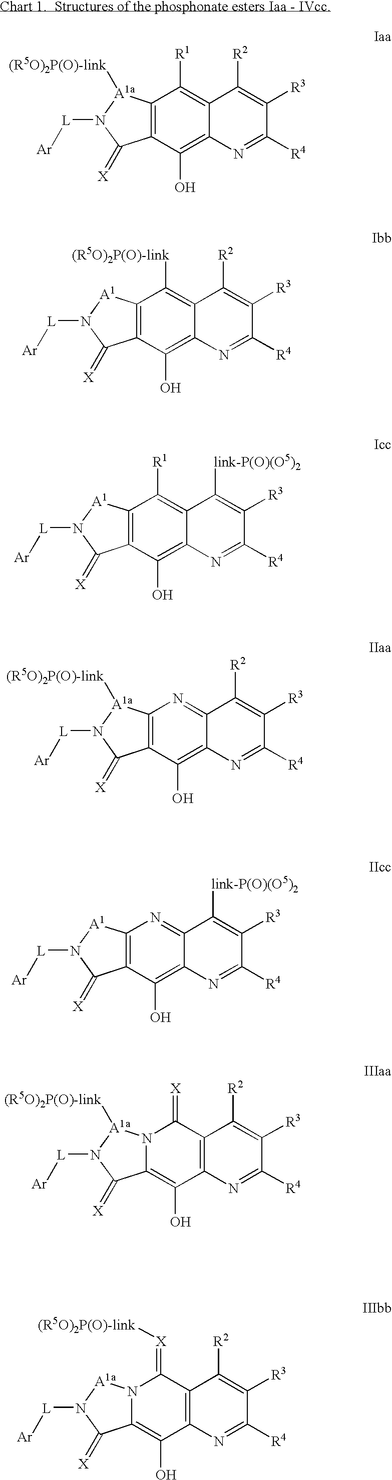 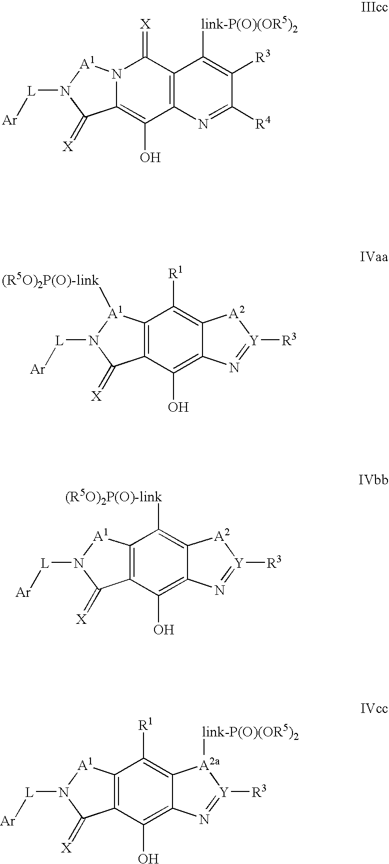 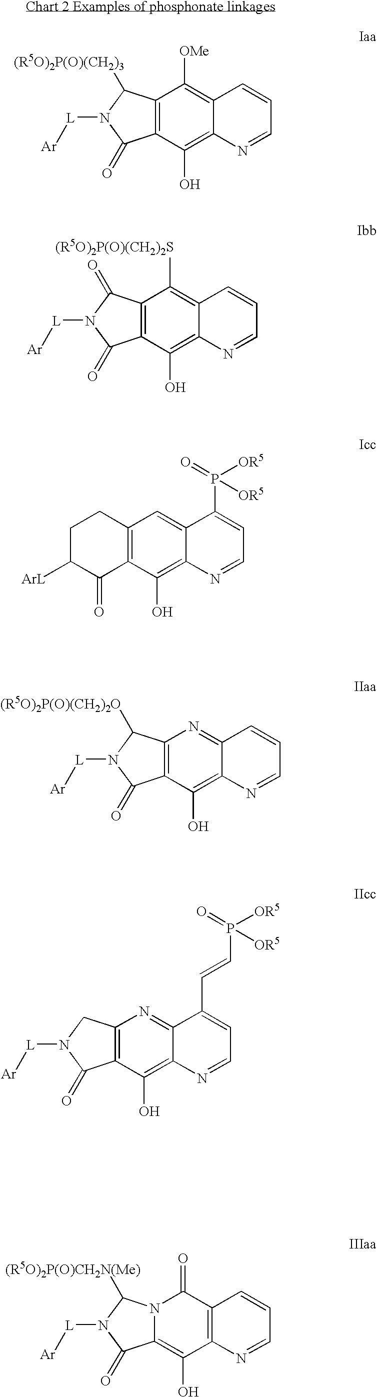 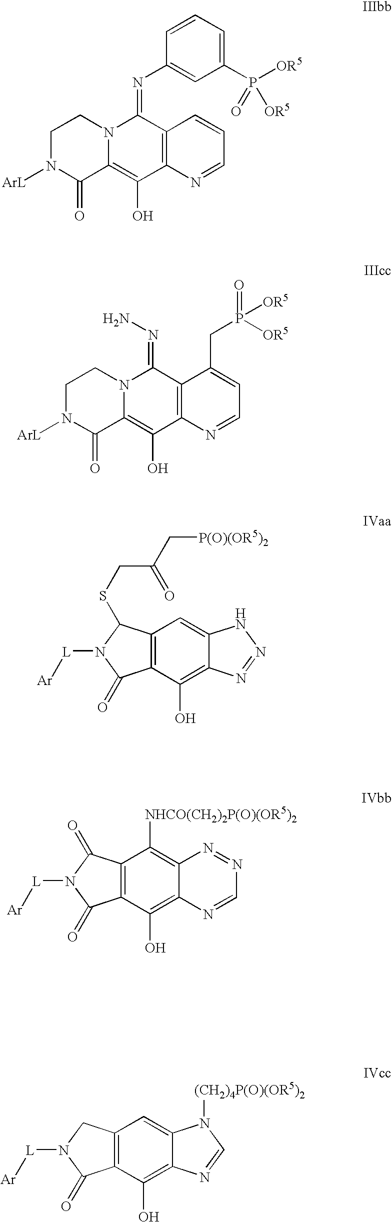
Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M Wuts, Wiley, Second Edition 1990. Reactive substituents which may be protected are shown in the accompanying schemes as, for example, [OH], [SH], etc.

Preparation of the Intermediate Phosphonate Esters 1aa.

Schemes A1-A5 illustrate methods for the preparation of the intermediate phosphonate esters Iaa.

As shown in Scheme A1, the phenolic hydroxyl substituent present in the tricyclic compound A1.1 is protected to afford the derivative A1.2. The protection of hydroxyl groups is described in Protective Groups in Organic Synthesis, by T. W. Greene and P. G. M Wuts, Wiley, Second Edition 1990, p. 10. For example, hydroxyl substituents are protected as trialkylsilyloxy, methoxymethyl, benzyl or tert-butyl ethers. Trialkylsilyl groups are introduced by the reaction of the phenol with a chlorotrialkylsilane and a base such as imidazole, for example as described in Protective Groups in Organic Synthesis, by T. W. Greene and P. G. M Wuts, Wiley, Second Edition 1990, p. 10ff. The protected product A1.2 is then reacted, in the presence of a strong base, with a bromoalkyl phosphonate A1.3, to give the alkylation product A1.4. The reaction is effected in a polar organic solvent such as dimethylformamide, dimethylacetamide, diglyme, tetrahydrofuran and the like, in the presence of a base such as sodium hydride, an alkali metal alkoxide, lithium hexamethyldisilazide, and the like, at from ambient temperature to about 100°, to yield the alkylated product A1.4. The phenolic hydroxyl group is then deprotected to afford the phenol A1.5. Methods for the deprotection of hydroxyl groups are described in Protective Groups in Organic Synthesis, by T. W. Greene and P. G. M Wuts, Wiley, Second Edition 1990, p. 10ff.

For example, 7-(4-fluoro-benzyl)-9-hydroxy-5H-1,7-diaza-anthracene-6,8-dione A1.6 is reacted with one molar equivalent of chlorotriisopropylsilane and imidazole in dimethylformamide at ambient temperature, as described in Tet. Lett., 2865, 1974, to produce 7-(4-fluoro-benzyl)-9-triisopropylsilanyloxy-5H-1,7-diaza-anthracene-6,8-dione A1.7. The product is then reacted in dimethylformamide solution at about 60° with one molar equivalent of a dialkyl 2-bromoethyl phosphonate A1.8 (Aldrich) and lithium hexamethyldisilazide, to yield the alkylated product A1.9. The silyl protecting group is then removed by reaction with tetrabutylammonium fluoride in tetrahydrofuran, as described in J. Org. Chem., 51, 4941, 1986, to give the phenolic product A1.10.

Using the above procedures, but employing, in place of the 4-fluorobenzyl-substituted phenol A1.6, different phenols A1.1 and/or different phosphonates A1.3, the corresponding products A1.5 are obtained.

Scheme A2 illustrates the preparation of phosphonate esters of structure Iaa in which the phosphonate group is attached by means of an aryl of heteroaryl ring.

In this procedure, a hydroxy-substituted phthalimide derivative A2.1 (Formula I) is protected, as described above, to afford the product A2.2. This compound is then reacted with a bromoaryl magnesium bromide Grignard reagent A2.3, in which the group Ar¹ is an aromatic or heteroaromatic group such as, for example, benzene or thiophene, to afford the alcohol A2.4. The regioselective addition of organometallic derivatives to phthalimides is described in Scheme 4. The reaction is performed between approximately equimolar amounts of the reactants in an ethereal solvent such as diethyl ether, tetrahydrofuran and the like, at from −40° C. to ambient temperature, to give the alcohol product A2.4. This material is then reacted with a dialkyl phosphite A2.5 and a palladium catalyst, to give the phosphonate A2.6. The preparation of arylphosphonates by means of a coupling reaction between aryl bromides and dialkyl phosphites is described in J. Med. Chem., 35, 1371, 1992. The reaction is conducted in a hydrocarbon solvent such as benzene, toluene or xylene, at about 100°, in the presence of a palladium (0) catalyst such as tetrakis(triphenylphosphine)palladium(0), and a tertiary base such as triethylamine or diisopropylethylamine. The hydroxyl group is then deprotected to yield the phenolic product A2.7. Optionally, the benzylic hydroxyl substituent in the product A2.7 is removed by means of a reductive procedure, as shown on Scheme 4. Benzylic hydroxyl groups are removed by catalytic hydrogenation, for example by the use of 10% palladium on carbon in the presence of hydrogen or a hydrogen donor, or by means of chemical reduction, for example employing triethylsilane and boron trifluoride etherate.

For example, 7-(3,5-dichloro-benzyl)-5,9-bis-triisopropylsilanyloxy-pyrrolo[3,4-g]quinoline-6,8-dione A2.9, prepared by silylation of the corresponding diol, which is reacted with one molar equivalent of 4-bromophenyl magnesium bromide A2.10 in ether at 0° to produce the alcohol A2.11. The latter compound is then reacted, in toluene solution at reflux, with a dialkyl phosphite A2.5, triethylamine and tetrakis(triphenylphosphine)palladium(0), as described in J. Med. Chem., 35, 1371, 1992, to afford the phosphonate product A2.12. Desilylation, for example by reaction with tetrabutyl ammonium fluoride, gives the diol product A2.13. Optionally, the product A2.12 is reduced, for example by reaction in dichloromethane solution at ambient temperature with ca. four molar equivalents of triethylsilane and boron trifluoride etherate, as described in Example 18 to yield after deprotection the reduced product A2.14.

Using the above procedures, but employing, in place of the 3,5-dichlorobenzyl-substituted phenol derivative A2.9, different phenol derivatives A2.1 and/or different bromoaryl Grignard reagents A2.3, the corresponding products A2.7 and A2.8 are obtained.

Scheme A3 illustrates the preparation of phosphonate esters of structure Iaa in which the phosphonate group is attached by means of an alkylene chain.

In this sequence, a 6-aminoquinoline ester A3.1, prepared, for example, from the corresponding carboxylic acid by means of a Curtius rearrangement, (Advanced Organic Chemistry, Part B, by F. A. Carey and R. J. Sundberg, Plenum, 2001, p. 646) is reacted, under reductive amination conditions, with a dialkyl formylalkyl phosphonate A3.2. The preparation of amines by means of reductive amination procedures is described, for example, in Comprehensive Organic Transformations, by R. C. Larock, VCH, p 421, and in Advanced Organic Chemistry, Part B, by F. A. Carey and R. J. Sundberg, Plenum, 2001, p 269. In this procedure, the amine component and the aldehyde or ketone component are reacted together in the presence of a reducing agent such as, for example, borane, sodium cyanoborohydride, sodium triacetoxyborohydride or diisobutylaluminum hydride, optionally in the presence of a Lewis acid, such as titanium tetraisopropoxide, as described in J. Org. Chem., 55, 2552, 1990. The product A3.3 is then converted, by reaction with the amine ArBNH₂ A3.4, or a derivative thereof, into the amide A3.5. The conversion of esters into amides is described in Comprehensive Organic Transformations, by R. C. Larock, VCH, 1989, p. 987. The reactants are combined in the presence of a base such as sodium methoxide under azeotropic conditions, or of a dialkyl aluminum or trialkyl tin derivative of the amine. The use of trimethylaluminum in the conversion of esters to amides is described in J. Med. Chem. Chim. Ther., 34, 1999, 1995, and Syn. Comm., 25, 1401, 1995. The reaction is conducted in an inert solvent such as dichloromethane or toluene. The amide product A3.5 is then cyclized by reaction with a reagent such as phosgene or a functional equivalent thereof, such as triphosgene or a dialkyl carbonate, or a reagent such as diiodomethane, to give the cyclized product A3.6 in which D is CO or CH₂. The reaction is conducted in an aprotic solvent such as tetrahydrofuran, in the presence of an inorganic or organic base such as potassium carbonate or diisopropylethylamine.

For example, the amine A3.7, prepared by means of a Curtius rearrangement of the corresponding MOM-protected carboxylic acid, is reacted in isopropanol solution with a dialkyl formylmethyl phosphonate A3.8, prepared as described in Zh. Obschei. Khim., 1987, 57, 2793, sodium cyanoborohydride and acetic acid, to give the reductive amination product A3.9. The product is then reacted with an excess of 3,4-dichlorobenzylamine and sodium methoxide in toluene at reflux, to yield the amide A3.10. The latter compound is then reacted with one molar equivalent of triphosgene and N,N-dimethylaminopyridine in dichloromethane, to afford the cyclized product A3.11. The MOM protecting groups are then removed, for example by reaction with a catalytic amount of methanolic hydrogen chloride, as described in J. Chem. Soc., Chem. Comm., 298, 1974, to give the dihydroxy product A3.12.

Using the above procedures, but employing, in place of the amine A3.7, different amines A3.1, and/or different aldehydes A3.2, and/or different amines A3.4, the corresponding products A3.6 are obtained.

Scheme A4 illustrates the preparation of phosphonate esters of structure Iaa in which the phosphonate group is attached by means of an alkylene chain or an aryl, heteroaryl or aralkyl group and a heteroatom O, S or N. In this sequence, a tricyclic aminal A4.1 is reacted in the presence of an acid catalyst with a hydroxy, mercapto or amino-substituted dialkyl phosphonate A4.2 in which X is O, S, NH or N-alkyl, and R is alkyl, alkenyl, aryl, heteroaryl or aralkyl. The reaction is effected at ambient temperature in an inert solvent such as dichloromethane, in the presence of an acid such as p-toluenesulfonic acid or trifluoroacetic acid and an excess of the reagent A4.2. The hydroxyl group is then deprotected to yield the phenolic product A4.4.

For example, 7-(4-fluoro-benzyl)-6-hydroxy-5-methoxy-9-triisopropylsilanyloxy-6,7-dihydro-pyrrolo[3,4-g]quinolin-8-one A4.5 (Example 20, Scheme 15) is reacted at ambient temperature in dichloromethane solution with a dialkyl 2-mercaptoethyl phosphonate A4.6 (Zh. Obschei. Khim., 1973, 43, 2364) and trifluoroacetic acid to give the thioether product A4.7, which upon deprotection with tetrabutylammonium fluoride yields the phenol A4.8.

As a further example, 6-hydroxy-5-methoxy-7-(4-trifluoromethyl-benzyl)-9-triisopropylsilanyloxy-6,7-dihydro-pyrrolo[3,4-g]quinolin-8-one A4.9, prepared analogously to the 4-fluoro analog A4.5, is reacted, under the same conditions, with a dialkyl 3-mercaptophenyl phosphonate A4.10 to give the thioether A4.11 which upon deprotection affords the phenol A4.12. The phosphonate reagent A4.10 is obtained by palladium (0) catalyzed coupling reaction, as described in Scheme A2, between a dialkyl phosphite and an S-protected derivative of 3-bromothiophenol, for example the S-trityl derivative, followed by removal of the sulfur protecting group. Protection and deprotection of thiols is described in Protective Groups in Organic Synthesis, by T. W. Greene and P. G. M Wuts, Wiley, Second Edition 1990, p. 277.

Using the above procedures, but employing, in place of the alcohols A4.5 and A4.9, different alcohols A4.1, and/or different alcohols, thiols or amines A4.2, the corresponding products A4.4 are obtained.

Scheme A5 illustrates the preparation of phosphonate esters of structure Iaa in which the phosphonate group is attached to a 7-membered ring by means of an alkylene or arylmethylene chain. In this sequence, a suitable protected quinoline acid ester A5.1 is subjected to a Curtius rearrangement, as described in Scheme A3 to yield the amine A5.2. The product is then reductively aminated, as described in Scheme A3, with a phosphonate aldehyde A5.3, in which the group R is an alkyl group or an aryl group, to give the amine product A5.4. This material is then coupled with the glycine derivative A5.5 to yield the amide A5.6. The preparation of amides from carboxylic acids and derivatives is described, for example, in Organic Functional Group Preparations, by S. R. Sandler and W. Karo, Academic Press, 1968, p. 274, and Comprehensive Organic Transformations, by R. C. Larock, VCH, 1989, p. 972ff. The carboxylic acid is reacted with the amine in the presence of an activating agent, such as, for example, dicyclohexylcarbodiimide or diisopropylcarbodiimide, optionally in the presence of, for example, hydroxybenztriazole, N-hydroxysuccinimide or N-hydroxypyridone, in a non-protic solvent such as, for example, pyridine, DMF or dichloromethane, to afford the amide. Alternatively, the carboxylic acid may first be converted into an activated derivative such as the acid chloride, anhydride, mixed anhydride, imidazolide and the like, and then reacted with the amine, in the presence of an organic base such as, for example, pyridine, to afford the amide. The conversion of a carboxylic acid into the corresponding acid chloride can be effected by treatment of the carboxylic acid with a reagent such as, for example, thionyl chloride or oxalyl chloride in an inert organic solvent such as dichloromethane, optionally in the presence of a catalytic amount of dimethylformamide. The product A5.6 is then cyclized, for example by heating at reflux temperature in toluene in the presence of a basic catalyst such as sodium methoxide, or by reaction with trimethylaluminum, as described in Syn. Comm., 25, 1401, 1995, to afford after deprotection of the hydroxyl groups, the diazepindione derivative A5.7.

For example, the MOM-protected amine A3.7 is reductively aminated by reaction with a dialkyl phosphonoacetaldehyde A5.8 (Aurora) and sodium triacetoxyborohydride, to produce the amine A5.9. The product is then coupled in dimethylformamide solution, in the presence of dicyclohexyl carbodiimide, with (4-fluoro-benzylamino)-acetic acid A5.10, to give the amide A5.11. This material is converted, by reaction with trimethylaluminum in dichloromethane, as described above, into the diazepin derivative A5.12. Removal of the MOM protecting groups, as previously described, then affords the phenolic product A5.13.

Using the above procedures, but employing, in place of the amine A3.7, different amines A5.2, and/or different aldehydes A5.3, and/or different carboxylic acids A5.5, the corresponding products A5.7 are obtained. 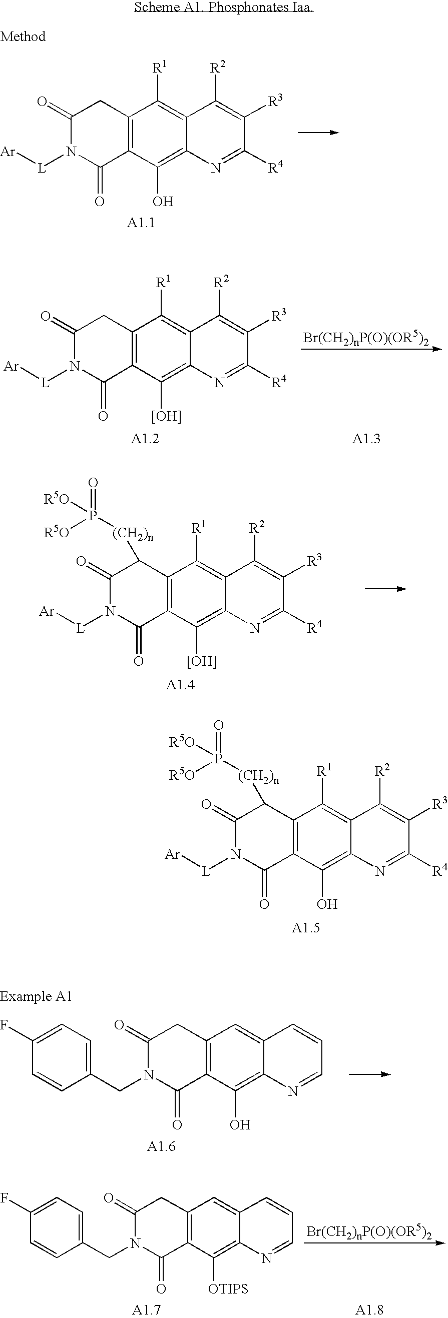 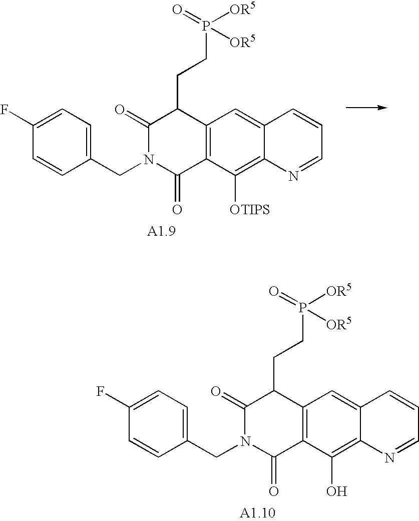 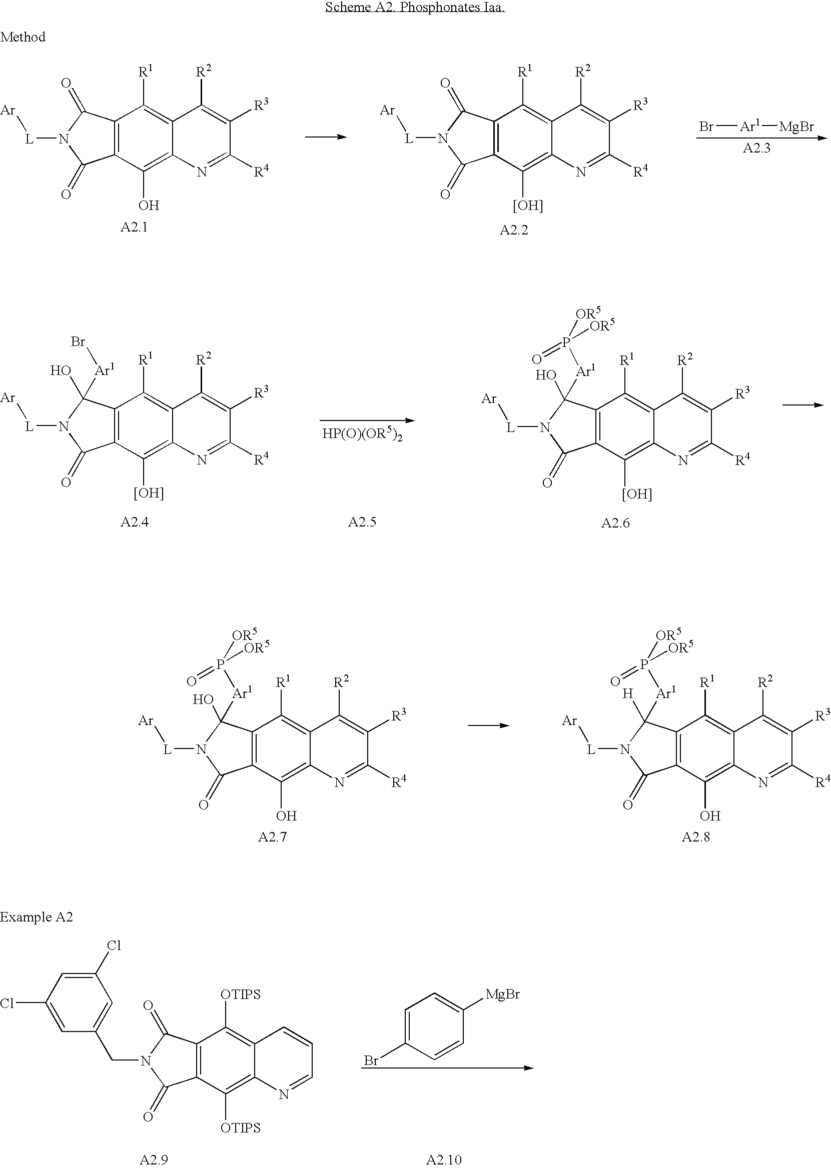 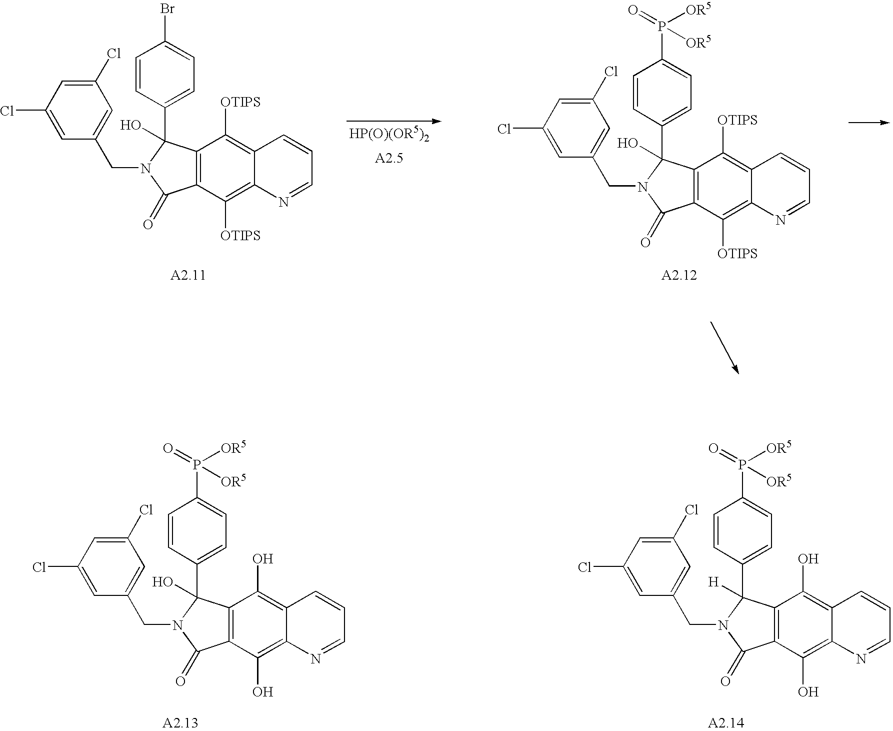 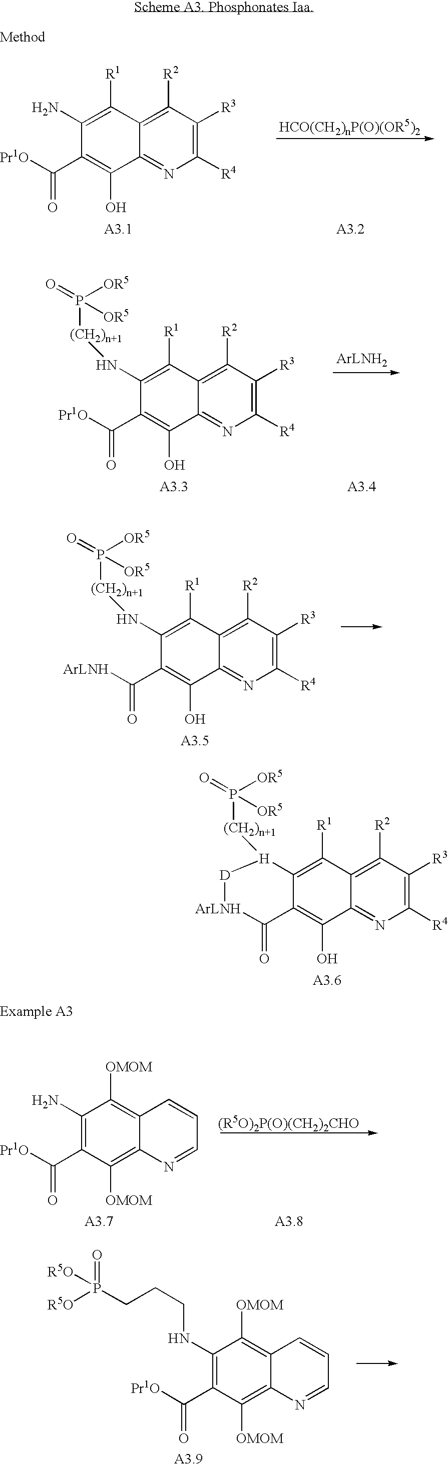 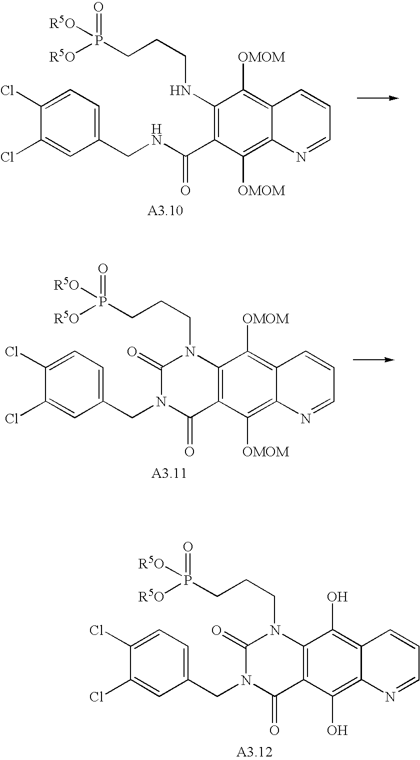 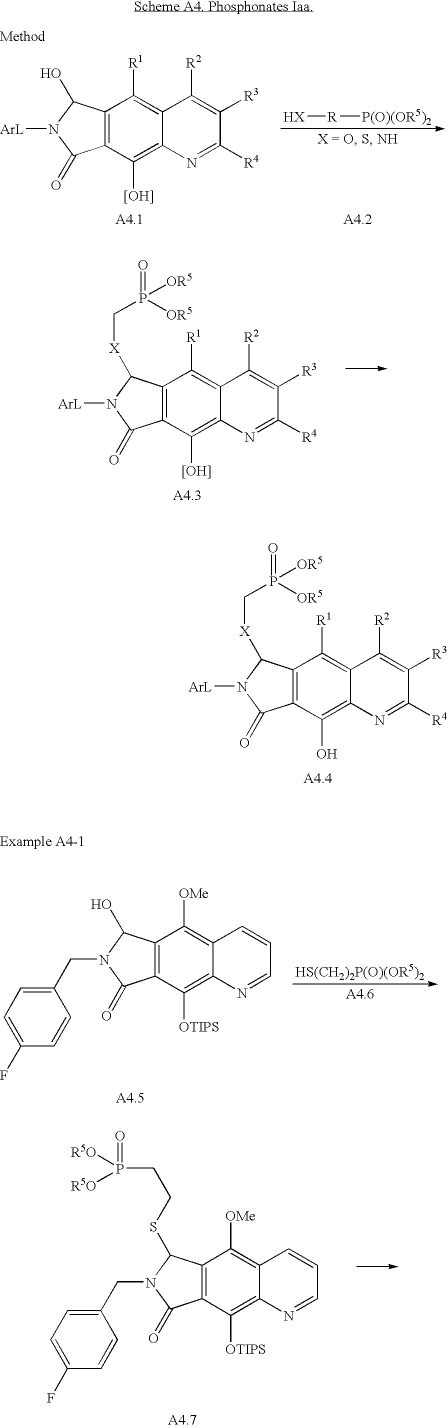 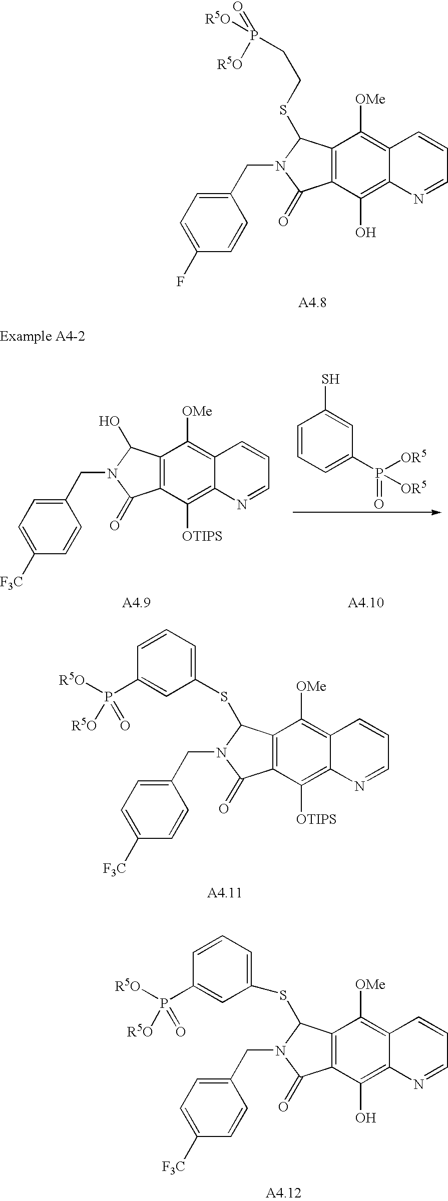 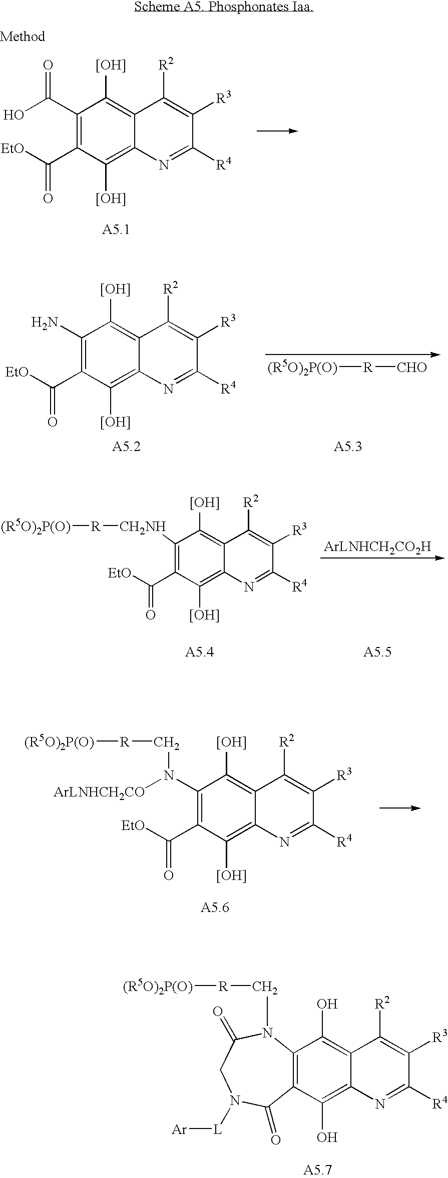 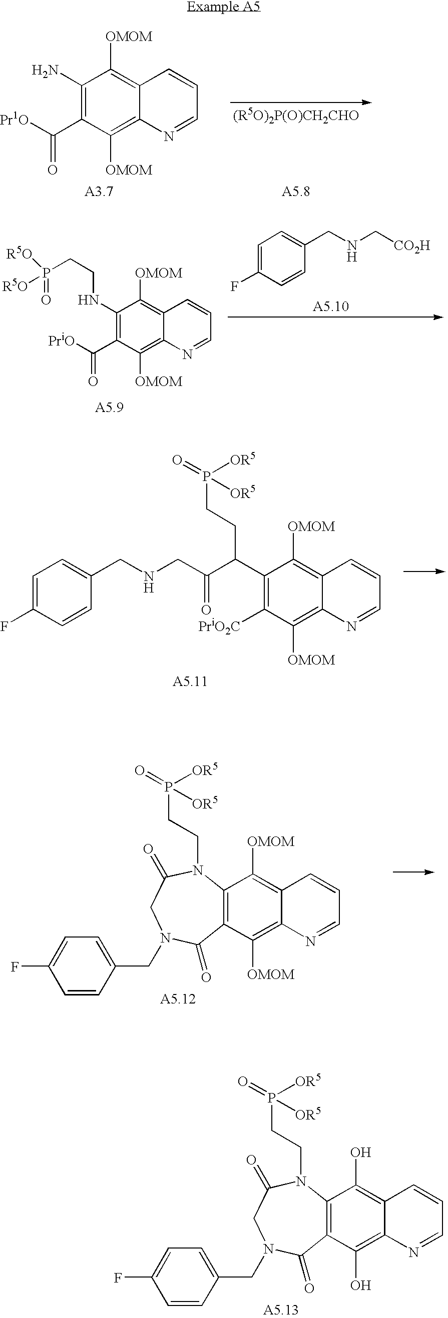
Preparation of the Intermediate Phosphonate Esters Ibb.

Schemes A6-A16 illustrate methods for the preparation of the phosphonate esters of general structure Ibb.

Scheme A6 depicts two methods for the preparation of phosphonate esters in which the phosphonate group is linked by means of a saturated or unsaturated alkylene chain, or alkylene chains incorporating carbocyclic, aryl or heteroaryl rings. In this procedure, a mono-protected phenol A6.1, for example, is reacted either with a bromo-substituted alkyl phosphonate A6.2, in which the group R is alkylene, cycloalkyl, alkenyl, aralkyl, heterarylalkyl and the like, or with an analogous hydroxyl-substituted dialkyl phosphonate A6.3. The reaction between the phenol and the bromo compound A6.2 is conducted in a polar organic solvent such as dimethylformamide, in the presence of a base such as potassium carbonate, and optionally in the presence of a catalytic amount of potassium iodide, to afford the ether product A6.4. Alternatively, the ether compounds A6.4 are obtained by means of a Mitsonobu reaction between the phenol A6.1 and the hydroxy compound A6.3. The preparation of aromatic ethers by means of the Mitsonobu reaction is described, for example, in Comprehensive Organic Transformations, by R. C. Larock, VCH, 1989, p. 448, and in Advanced Organic Chemistry, Part B, by F. A. Carey and R. J. Sundberg, Plenum, 2001, p. 153-4 and in Org. React., 1992, 42, 335. The phenol and the alcohol component are reacted together in an aprotic solvent such as, for example, tetrahydrofuran, in the presence of a dialkyl azodicarboxylate and a triarylphosphine, to afford the ether or thioether products. The procedure is also described in Org. React., 1992, 42, 335-656. Deprotection of the phenolic hydroxyl group then affords the phenol A6.5.

For example, 7-(4-fluoro-benzyl)-5-hydroxy-9-triethylsilanyloxy-pyrrolo[3,4-g]quinoline-6,8-dione A6.6, (Example 12, Scheme 11) is reacted at ambient temperature in dimethoxyethane solution with one molar equivalent of a dialkyl 4-bromo-2-butenylphosphonate A6.7 (J. Med. Chem., 1992, 35, 1371) and potassium carbonate, to yield the ether product A6.8, which upon deprotection with tetrabutylammonium fluoride gives the phenol A6.9.

As a further example, 7-[2-(4-fluoro-phenyl)-ethyl]-5-hydroxy-9-triethylsilanyloxy-pyrrolo[3,4-g]quinoline-6,8-dione A6.10 prepared by analogous procedures to those shown is reacted in tetrahydrofuran solution with a dialkyl 3-hydroxypropyl phosphonate A6.11 (Acros), diethyl azodicarboxylate and triphenylphosphine, to afford the ether product A6.12 which upon deprotection gives the phenol A6.13.

Using the above procedures, but employing, in place of the phenols A6.6 and A6.10, the phenols A6.1, and/or different bromides A6.2, or alcohols A6.3, the corresponding products A6.5 are obtained.

Scheme A7 illustrates the preparation of phosphonate esters of structure Ibb in which the phosphonate is linked by means of an aryl or a heteroaryl group.

In this procedure, a mono-protected phenol A7.1 (Formula I) is converted into the triflate A7.2 by reaction, in an inert solvent such as dichloromethane, with trifluoromethanesulfonyl chloride or anhydride, or with trimethylsilyl triflate and triethylsilane, in each case in the presence of a tertiary base such as triethylamine. The triflate is then coupled with a bromo-substituted arylboronate A7.3, in which the group Ar¹ is an aromatic or heteroaromatic moiety, to afford the coupled product A7.4. The Suzuki coupling of aryl triflates and aryl boronic acids is described in Palladium Reagents and Catalysts by J. Tsuji, Wiley 1995, p 218. The reactants are combined in an inert solvent such as toluene or dioxan, in the presence of a palladium (0) catalyst such as tetrakis(triphenylphosphine)palladium and a base such as sodium bicarbonate. The coupled product A7.4 is then reacted, as described previously (Scheme A2) with a dialkyl phosphite A7.5, to give the phosphonate ester A7.6, which upon deprotection yields the phenol A7.7.

For example, trifluoro-methanesulfonic acid 9-benzhydryloxy-7-(4-fluoro-benzyl)-8-oxo-7,8-dihydro-6H-pyrrolo[3,4-g]quinolin-5-yl ester A7.8 (Example 46) is reacted in dioxan solution at 70° with one molar equivalent of 3-bromophenyl boronic acid A7.9 (Maybridge), sodium bicarbonate and a catalytic amount of tri-(o-tolyl)phosphine, to produce the coupled compound A7.10. This material is then reacted, as described in Scheme A2, with a dialkyl phosphite and a palladium (0) catalyst, to give the phosphonate product A7.10. Removal of the benzhydryl protecting group, for example by treatment with trifluoroacetic acid and anisole in dichloromethane, as described in Tet. Lett., 25, 3909, 1984, then affords the phenol A7.11.

Using the above procedures, but employing, in place of the phenol A7.8, the phenol A7.1, and/or different boronic acids A7.3, the corresponding products A7.7 are obtained.

Scheme A8 illustrates the preparation of phosphonate esters of structure Ibb in which the phosphonate group is linked by means of an oxygen, sulfur or nitrogen and an aliphatic or aromatic moiety.

In this method, a monoprotected phenol A8.1 (Formula I) is converted into the corresponding triflate A8.2, as described above (Scheme A7). The product is then subjected to a nucleophilic displacement reaction with various alcohols, thiols or amines A8.3, in which the group R is an acyclic or cyclic saturated or unsaturated alkylene, or aryl, aralkyl or heteroaryl moiety, to afford after deprotection the ether, thioether or amine products A8.4. The displacement reaction is performed in an inert solvent such as dichloroethane or dioxan, at from ambient temperature to about 80°, in the presence of a tertiary organic base such as N-methyl morpholine and the like.

For example, trifluoro-methanesulfonic acid 9-benzhydryloxy-7-(4-fluoro-benzyl)-6,8-dioxo-7,8-dihydro-6H-pyrrolo[3,4-g]quinolin-5-yl ester A8.5 (Example 56) is reacted in dioxan at 50° with one molar equivalent of a dialkyl methylaminomethyl phosphonate A8.6 and diisopropylethylamine, to give the amine product A8.7. Deprotection then affords the phenol A8.8.

Using the above procedures, but employing, in place of the triflate A8.5, different triflates A8.2, and/or different alcohols, thiols or amines A8.3, the corresponding products A8.4 are obtained.

Scheme A9 depicts the preparation of phosphonate esters of structure Ibb in which the phosphonate group is attached by means of an methylamino group and a carbon link R, in which the group R is an acyclic or cyclic saturated or unsaturated alkylene, or aryl, aralkyl or heteroaryl moiety. The compounds are obtained by means of a reductive alkylation reaction, as described above (Scheme A3) between the aldehyde A9.1, prepared by the method shown in Example 49, and a dialkyl aminoalkyl or aryl phosphonate A9.2. The amination product A9.3 is then deprotected to give the phenol A9.3.

For example, 9-benzhydryloxy-7-(4-fluoro-benzyl)-8-oxo-7,8-dihydro-6H-pyrrolo[3,4-g]quinoline-5-carbaldehyde A9.5 (Example 49) is reacted with a dialkyl aminopropyl phosphonate A9.6 (Acros), sodium cyanoborohydride and acetic acid in isopropanol to yield the amination product A9.7, which is deprotected to produce the phenol A9.8.

Using the above procedures, but employing, in place of the aldehyde A9.5, different aldehydes A9.1, and/or different amines A9.2, the corresponding products A9.4 are obtained.

Scheme A10 depicts the preparation of phosphonate esters of structure Ibb in which the phosphonate group is attached by means of an amide linkage and a carbon link R, in which the group R is an acyclic or cyclic saturated or unsaturated alkylene, or aryl, aralkyl or heteroaryl moiety. In this sequence, the aldehyde A10.1, prepared, for example, as shown in Example 49 is oxidized to the corresponding carboxylic acid A10.2. The conversion of an aldehyde to the corresponding carboxylic acid is described in Comprehensive Organic Transformations, by R. C. Larock, VCH, 1989, p. 838. The reaction is effected by the use of various oxidizing agents such as, for example, potassium permanganate, ruthenium tetroxide, silver oxide or sodium chlorite. The carboxylic acid is then coupled, as described in Scheme A5, with an amine A10.3 to afford the amide, which upon deprotection gives the phenolic amide A10.4.

For example, 9-benzhydryloxy-7-(4-chloro-benzyl)-6,8-dioxo-7,8-dihydro-6H-pyrrolo[3,4-g]quinoline-5-carbaldehyde A10.5, prepared using the methods described in Example 49, is treated with silver oxide in acetonitrile, as described in Tet. Lett., 5685, 1968, to produce the corresponding carboxylic acid 9-benzhydryloxy-7-(4-chloro-benzyl)-6,8-dioxo-7,8-dihydro-6H-pyrrolo[3,4-g]quinoline-5-carboxylic acid A10.6. This material is then coupled, in dimethylformamide solution, with one molar equivalent of a dialkyl aminoethyl phosphonate A10.7 (Aurora) and dicyclohexyl carbodiimide, to afford the amide, which upon deprotection gives the phenolic product A10.8.

Using the above procedures, but employing, in place of the aldehyde A10.5, different aldehydes A10.1, and/or different amines A10.3, the corresponding products A10.4 are obtained.

Scheme A11 depicts the preparation of phosphonate esters of structure Ibb in which the phosphonate group is attached by means of a methylene group. In this procedure, a hydroxymethyl-substituted O-protected phenol A11.1, prepared by the method shown in Example 50, is converted into the corresponding bromomethyl derivative A11.2. The conversion of alcohols into the corresponding bromides is described, for example, in Comprehensive Organic Transformations, by R. C. Larock, VCH, 1989, p. 356ff. For example, benzyl alcohols can be transformed into the bromo compounds by reaction with bromine and triphenyl phosphite, or by reaction with trimethylsilyl chloride and lithium bromide, or with carbon tetrabromide and triphenylphosphine, as described in J. Am. Chem. Soc., 92, 2139, 1970. The resultant bromomethyl compound A11.2 is treated with a trialkyl phosphite A11.3 in an Arbuzov reaction. The preparation of phosphonates by means of the Arbuzov reaction is described in Handb. Organophosphorus Chem., 1992, 115-72. The bromo compound is heated with an excess of the phosphite at from about 80°-130° to produce the phosphonate product, which upon deprotection affords the phenolic phosphonate A11.4.

For example, 9-benzhydryloxy-5-hydroxymethyl-7-(4-methoxy-benzyl)-6,7-dihydro-pyrrolo[3,4-g]quinolin-8-one A11.5 prepared by the method shown in Example 50, is reacted in dichloromethane with one molar equivalent of carbon tetrabromide and triphenylphosphine to produce 9-benzhydryloxy-5-bromomethyl-7-(4-methoxy-benzyl)-6,7-dihydro-pyrrolo[3,4-g]quinolin-8-one A11.6. The product is then heated at 120° with an excess of a trialkyl phosphite A11.3. The resulting phosphonate is then deprotected to afford the phenolic product A11.7.

Using the above procedures, but employing, in place of the alcohol A11.5, different alcohols A11.1, and/or different phosphites A11.3, the corresponding products A11.4 are obtained.

Scheme A12 depicts the preparation of phosphonate esters of structure Ibb in which the phosphonate group is attached by means of a methyleneoxy and a variable alkyl moiety. In this procedure, a protected hydroxymethyl-substituted tricyclic phenol A12.1 prepared according to the procedure of Example 50, is alkylated with a dialkyl bromo-substituted phosphonate A12.2, in which the group R is an acyclic or cyclic saturated or unsaturated alkylene, or aryl, aralkyl or heteroaryl moiety. The alcohol is reacted with one molar equivalent of the bromo compound in a polar aprotic organic solvent such as dimethylacetamide, dioxan and the like, in the presence of a strong base such as sodium hydride, lithium hexamethyldisilazide, or potassium tert.-butoxide. The thus-obtained ether A12.3 is then deprotected to give the phenol A12.4.

For example, 9-benzhydryloxy-7-(4-fluoro-benzyl)-5-hydroxymethyl-6,7-dihydro-pyrrolo[3,4-g]quinolin-8-one A12.5 (Example 50) is treated in dimethylformamide solution at ambient temperature with one molar equivalent of lithium hexamethyldisilazide, followed by one molar equivalent of a dialkyl 4-(bromomethyl)benzyl phosphonate A12.6 (Tet., 1998, 54, 9341) to yield the alkylated product A12.7. Deprotection then gives the phenol A12.8.

Using the above procedures, but employing, in place of the alcohol A12.5, different alcohols A12.1, and/or different bromo compounds A12.2, the corresponding products A12.4 are obtained.

Scheme A13 depicts the preparation of phosphonate esters of structure Ibb in which the phosphonate group is attached by means of an aryl or heteroaryl ethenyl or ethyl linkage. In this procedure, a vinyl-substituted OH-protected phenol A13.1, prepared by the method shown in Example 59, is coupled in a palladium-catalyzed Heck reaction with a dibromo-substituted aromatic or heteroaromatic reagent A13.2, in which the group Ar¹ is an aromatic or heteroaromatic ring. The coupling of aryl halides with olefins by means of the Heck reaction is described, for example, in Advanced Organic Chemistry, by F. A. Carey and R. J. Sundberg, Plenum, 2001, p. 503ff and in Acc. Chem. Res., 12, 146, 1979. The aryl bromide and the olefin are coupled in a polar solvent such as dimethylformamide or dioxan, in the presence of a palladium(0) catalyst such as tetrakis(triphenylphosphine)palladium(0) or a palladium(II) catalyst such as palladium(II) acetate, and optionally in the presence of a base such as triethylamine or potassium carbonate. The coupled product A13.3 is then reacted, as described in Scheme A7, with a dialkyl phosphite A13.4 and a palladium catalyst, to afford, after deprotection of the phenolic hydroxyl, the ethenyl phosphonate ester A13.5. Catalytic or chemical reduction of the product then yields the saturated analog A13.6. The reduction reaction is effected chemically, for example by the use of diimide or diborane, as described in Comprehensive Organic Transformations, by R. C. Larock, VCH, 1989, p. 5, or catalytically, for example by the use of a palladium on carbon catalyst in the presence of hydrogen or a hydrogen donor.

For example, 9-benzhydryloxy-7-(4-fluoro-benzyl)-5-vinyl-6,7-dihydro-pyrrolo[3,4-g]quinolin-8-one A13.7 (Example 59) is reacted in dimethylformamide with 2,5-dibromothiophene A13.8 and a catalytic amount of palladium (II) acetate and triethylamine, to give the coupled product A13.9. This material is then coupled with a dialkyl phosphite, as described above, to afford after deprotection of the phenol, the ethenylthienyl phosphonate A13.10. The latter compound is reacted with diimide, prepared by basic hydrolysis of diethyl azodicarboxylate, as described in Angew. Chem. Int. Ed., 4, 271, 1965, to yield the saturated product A13.11.

Using the above procedures, but employing, in place of the vinyl-substituted compound A13.7, different analogs A13.1, and/or different dibromo compounds A13.2, the corresponding products A13.5 are obtained.

Scheme A14 depicts the preparation of phosphonate esters of structure Ibb in which the phosphonate group is attached by means of an alkoxy chain incorporating an amide linkage. In this procedure, a mono-protected phenol A14.1 (Example 6) is alkylated with a methyl bromoalkyl carboxylate A14.2. The alkylation reaction is conducted under similar conditions to those described in Scheme A6, to afford the ester ether A14.3. Hydrolysis of the ester group then gives the carboxylic acid A14.4. Hydrolysis methods for converting esters into carboxylic acids are described, for example, in Comprehensive Organic Transformations, by R. C. Larock, VCH, 1989, p 981. The methods include the use of enzymes such as pig liver esterase, and chemical methods such as the use of alkali metal hydroxides in aqueous organic solvent mixtures, for example lithium hydroxide in an aqueous organic solvent.

The resultant carboxylic acid is then coupled, as described in Scheme A10, with a dialkyl amino-substituted phosphonate A14.5, in which the group R is an acyclic or cyclic saturated or unsaturated alkylene, or aryl, aralkyl or heteroaryl moiety, to produce the amide A14.6. Deprotection then yields the phenol A14.7.

For example, 5-hydroxy-9-methoxymethoxy-7-(4-methyl-benzyl)-pyrrolo[3,4-g]quinoline-6,8-dione A14.8, prepared, for example, by the method shown in Example 6 is reacted in dimethylformamide solution with methyl bromoacetate A14.9 and cesium carbonate, to give the ether A14.10. The ester group is then hydrolyzed by reaction with one molar equivalent of lithium hydroxide in aqueous glyme, to produce the carboxylic acid A14.11. The carboxylic acid is then coupled in dimethylformamide solution in the presence of diisopropyl carbodiimide with a dialkyl 2-aminoethyl phosphonate A14.12, (J. Org. Chem., 2000, 65, 676) to form the amide A14.13. Deprotection, for example by the use of 50% aqueous acetic acid containing a catalytic amount of sulfuric acid, as described in J. Am. Chem. Soc., 55, 3040, 1933, then affords the phenol A14.14.

Using the above procedures, but employing, in place of the phenol A14.8, different phenols A14.1, and/or different bromoesters A14.2, and/or different amines A14.5, the corresponding products A14.7 are obtained.

Scheme A15 depicts the preparation of phosphonate esters of structure Ibb in which the phosphonate group is attached by means of an alkylene chain incorporating an amide linkage. In this procedure, the malonic ester derivative of a protected phenol A15.1, prepared, for example, by the methods shown in Example 86, is hydrolyzed and decarboxylated to give the corresponding acetic acid derivative A15.2. Hydrolysis and decarboxylation of malonic esters is described, for example, in Advanced Organic Chemistry, Part B, by F. A. Carey and R. J. Sundberg, Plenum, 2001, p. 15. The ester hydrolysis is effected under conventional basic conditions, and decarboxylation occurs after acidification either spontaneously or under mild heating. The resultant acetic acid derivative is then coupled, as described previously, with a dialkyl amino-substituted phosphonate A15.3, to give the amide product which upon deprotection affords the phenol A15.4.

For example, 2-[9-benzhydryloxy-7-(4-fluoro-benzyl)-8-oxo-7,8-dihydro-6H-pyrrolo[3,4-g]quinolin-5-yl]-malonic acid dimethyl ester A15.5 (Example 86) is reacted at ambient temperature with two molar equivalents of lithium hydroxide in aqueous dimethoxyethane, and the reaction mixture is then acidified to pH 4.0 and heated at reflux to effect decarboxylation and production of the acetic acid derivative A15.6. The carboxylic acid is then coupled in acetonitrile solution in the presence of a water-soluble carbodiimide with a dialkyl 4-aminophenyl phosphonate A15.7 (Epsilon) to yield after deprotection the phenolic amide A15.8.

Using the above procedures, but employing, in place of the malonic ester A15.5, different malonic esters A15.1, and/or different amines A15.3, the corresponding products A15.4 are obtained.

Scheme A16 depicts the preparation of phosphonate esters of structure Ibb in which the phosphonate group is attached by means of an alkoxy chain and the nucleus incorporates a benzazepin moiety. In this procedure, a quinoline monoester A16.1 is decarboxylated to afford the ester A16.2. Decarboxylation of carboxylic acids is described in Advanced Organic Chemistry, Part B, by F. A. Carey and R. J. Sundberg, Plenum, 2001, p. 676 and in Advanced Organic Chemistry, By J. Marsh, McGraw Hill, 1968, p. 435. The carboxylic acid is decarboxylated thermally in the presence of copper powder and quinoline, or by conversion to an ester with N-hydroxyphthalimide or N-hydroxythiopyridine, followed by photolysis in the presence of a hydrogen donor. The decarboxylated product A16.2 is then converted into the allyl ether A16.3 by reaction with allyl bromide in a polar solvent such as dimethylformamide in the presence of a base such h as triethylaamine or potassium carbonate. The allyl ester is then subjected to a thermal Claisen rearrangement to afford the allyl-substituted phenol A16.4. The Claisen rearrangement of allyl aryl ethers is described in Advanced Organic Chemistry, By J. Marsh, McGraw Hill, 1968, p. 830 and in Advanced Organic Chemistry, Part B, by F. A. Carey and R. J. Sundberg, Plenum, 2001, p. 394. The reaction is conducted in a high-boiling solvent or without solvent at ca. 200°. The free phenolic hydroxyl group is then protected to yield the doubly protected product A16.5. The latter compound is then subjected to a hydroboration procedure to afford the alcohol A16.6. Hydroboration of alkenes is described, for example, in Advanced Organic Chemistry, Part B, by F. A. Carey and R. J. Sundberg, Plenum, 2001, p. 226. The olefin is reacted with diborane or a substituted borane such as 9-BBN or catechyl borane, and the resulting borane is oxidized, for example with hydrogen peroxide, oxygen, sodium peroxycarbonate or a tertiary amine oxide. The resultant alcohol A16.6 is then converted into the substituted amine A16.7. The conversion is effected in two stages. In the first step, the alcohol is converted into a leaving group such as mesylate, tosylate or bromide by reaction with, for example, methanesulfonyl chloride, p-toluenesulfonyl chloride or triphenylphosphine/carbon tetrabromide. In the second step, the activated intermediate is reacted in a polar solvent such as N-methylpyrrolidinone or acetonitrile with the amine ArBNH₂ to give the product A16.7. The aminoester is then cyclized to yield the azepin derivative A16.8. The cyclization reaction is performed under similar conditions to those described above (Scheme A5). For example, the aminoester is heated in xylene at reflux temperature in the presence of a catalytic amount of sodium isopropoxide. The doubly protected azepin derivative A16.8 is then selectively deprotected to give the phenol A16.9. The procedure for the selective deprotection is dependent on the nature of the protecting groups. For example, if the phenol A16.1 is protected as the benzhydryl derivative, the phenol A16.4 is protected as, for example, the TIPS derivative. Deprotection of the azepin A16.8 is then effected by treatment with tetrabutylammonium fluoride in tetrahydrofuran. The phenol A16.9 is then reacted with a dialkyl hydroxy-substituted phosphonate A16.10, in which the group R is an alkylene or alkenyl chain, optionally incorporating an aryl or heteroaryl group. The reaction is performed under the conditions of the Mitsonobu reaction, as described in Scheme A6. The resultant ether is then deprotected to afford the phenol A16.11.

For example, 8-benzhydryloxy-7-methyl-quinolin-5-ol A16.12 prepared as described above from the corresponding carboxyester is converted, via allylation, rearrangement and hydroboration/oxidation, as described above, into 3-(8-benzhydryloxy-7-methyl-5-triisopropylsilanyloxy-quinolin-6-yl)-propan-1-ol A16.13. The latter compound is then converted into an activated derivative which is reacted, as described above, with 3-chloro-4-fluorobenzylamine A16.14 to yield [3-(8-benzhydryloxy-7-methyl-5-triisopropylsilanyloxy-quinolin-6-yl)-propyl]-(3-chloro-4-fluoro-benzyl)-amine A16.15. Cyclization of the product, for example by reaction with trimethylaluminum, employing the conditions described above, affords 11-benzhydryloxy-9-(3-chloro-4-fluoro-benzyl)-5-triisopropylsilanyloxy-6,7,8,9-tetrahydro-1,9-diaza-cyclohepta[b]naphthalen-10-one A16.16. The compound is deprotected by reaction with tetrabutylammonium fluoride, to produce 11-benzhydryloxy-9-(3-chloro-4-fluoro-benzyl)-5-hydroxy-6,7,8,9-tetrahydro-1,9-diaza-cyclohepta[b]naphthalen-10-one A16.17. The product is then reacted with a dialkyl hydroxyethyl phosphonate A16.18, diethyl azodicarboxylate and triphenylphosphine in tetrahydrofuran to give after deprotection the phenolic ether A16.19.

Using the above procedures, but employing, in place of the phenol A16.12, different phenols A16.2, and/or different hydroxyesters A16.10, and/or different amines ArBNH₂, the corresponding products A16.11 are obtained. 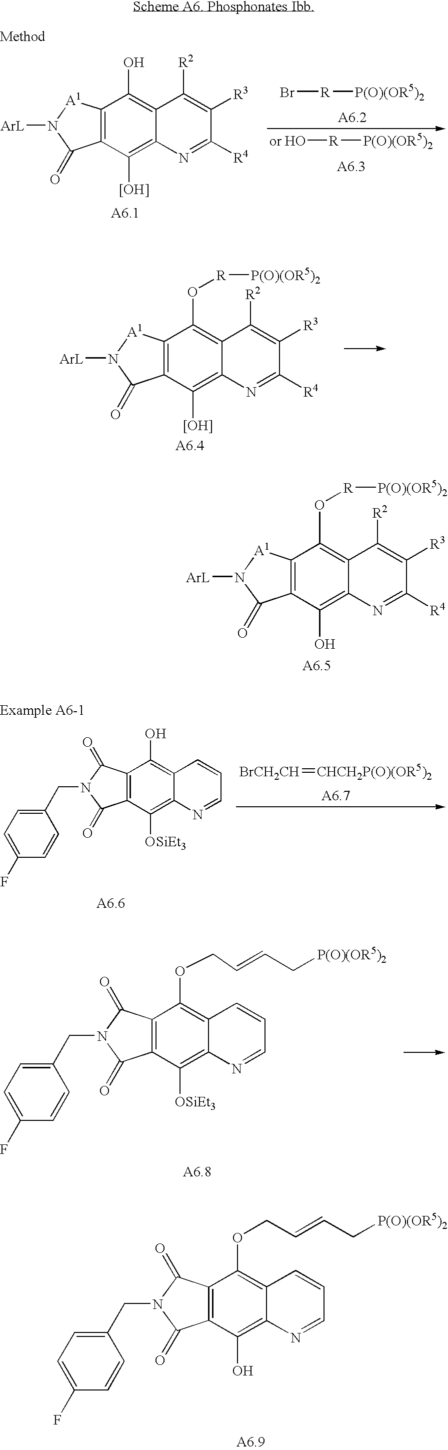 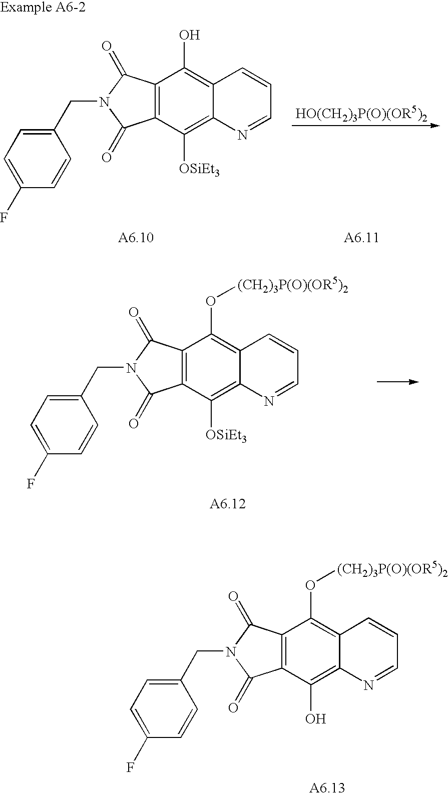 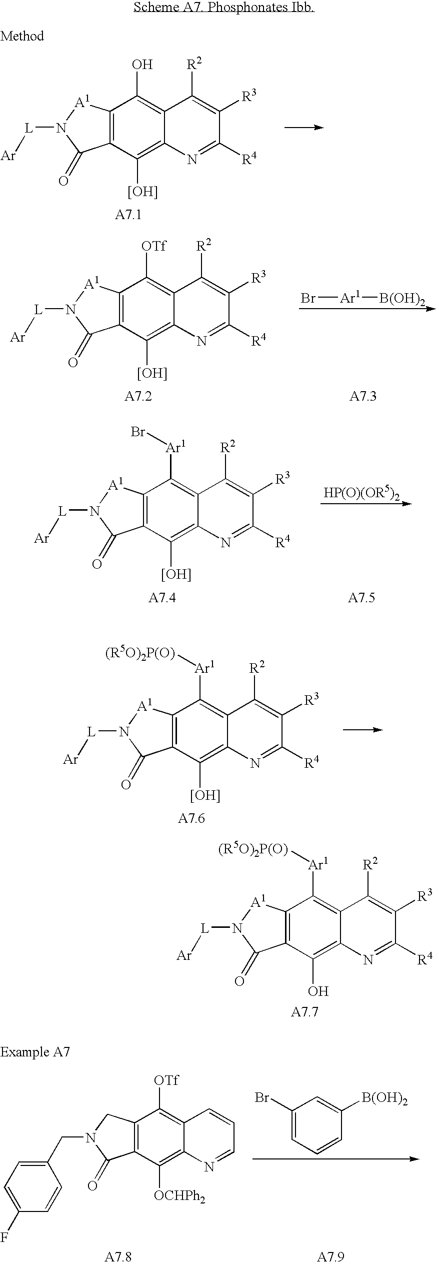 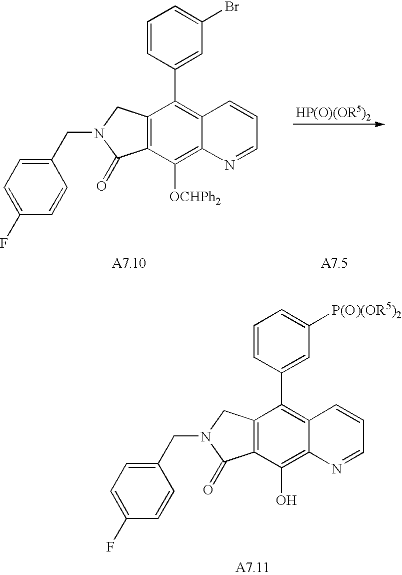 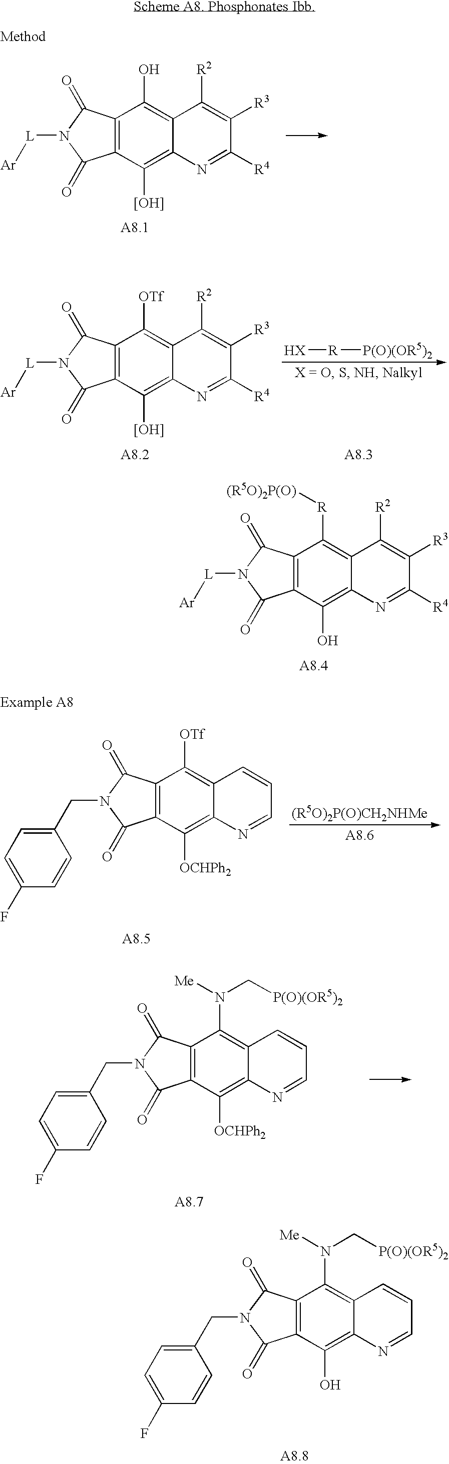 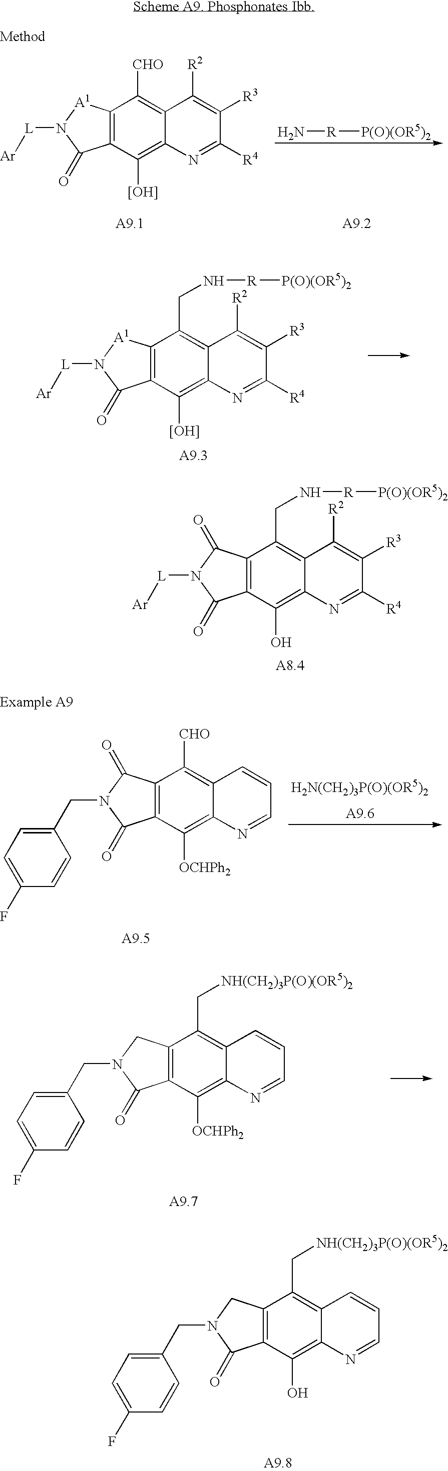 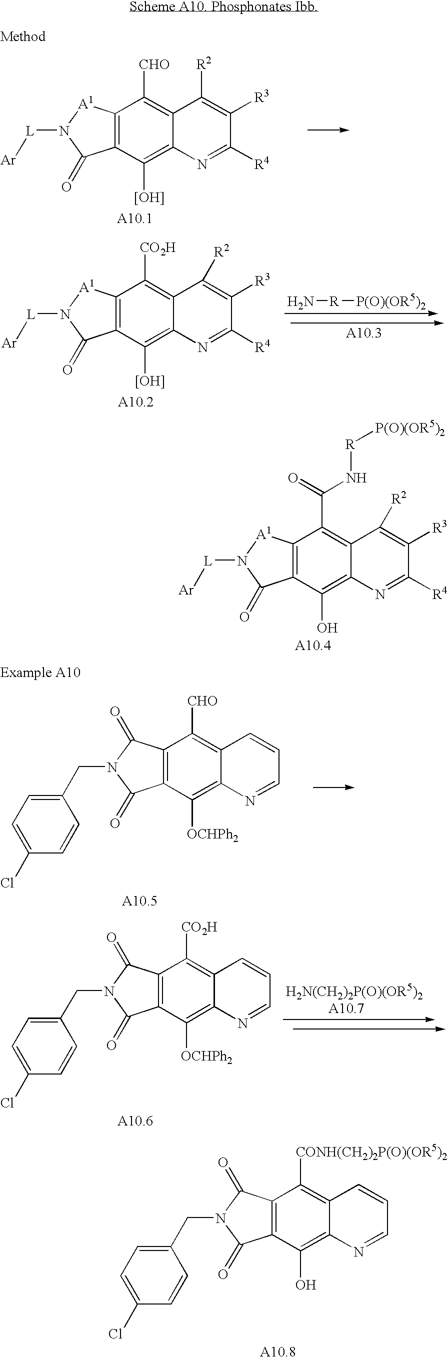 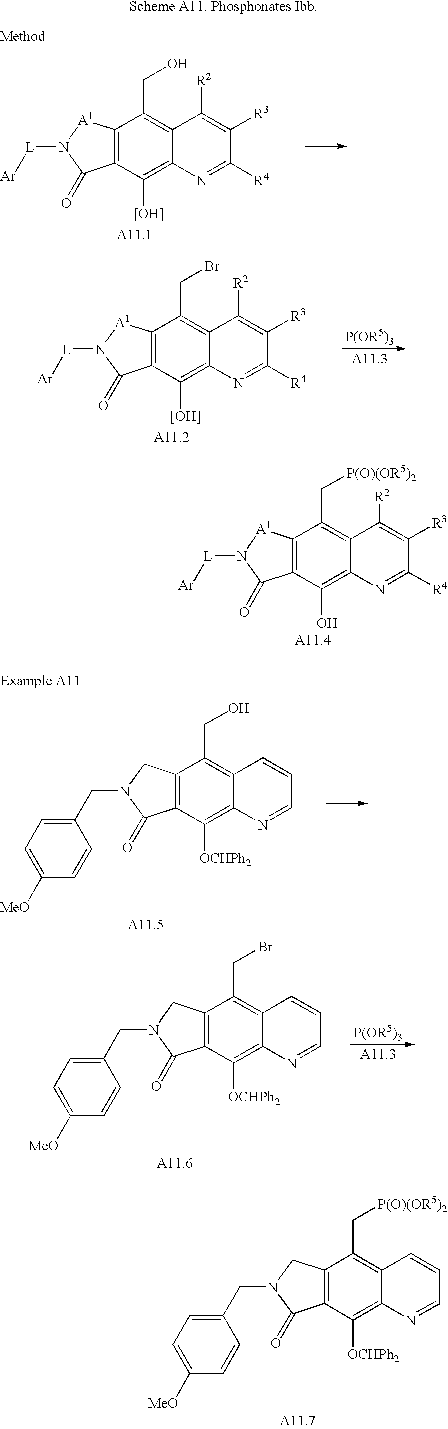 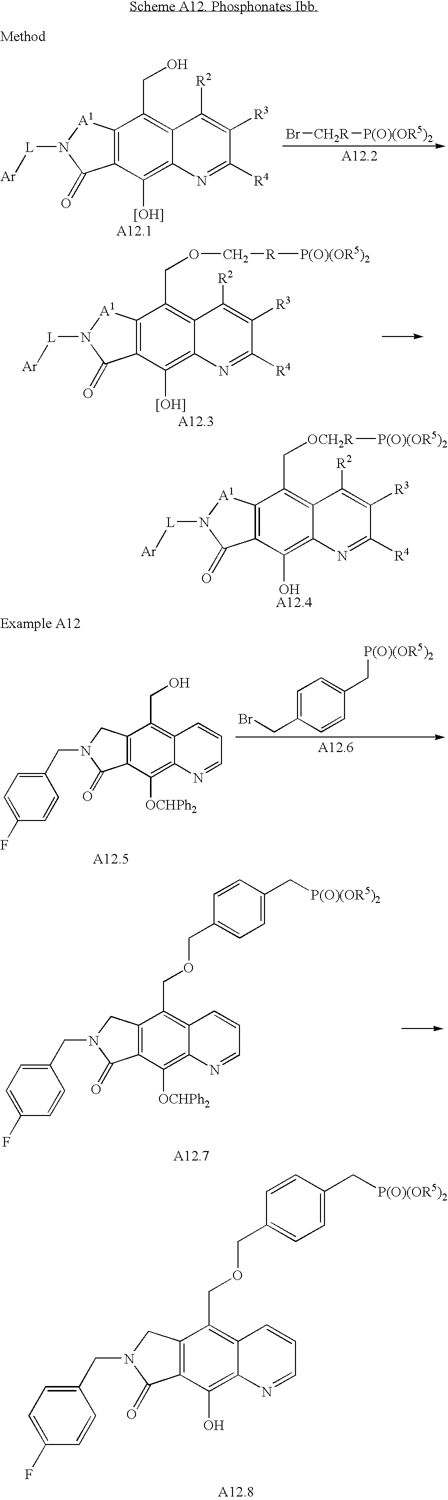 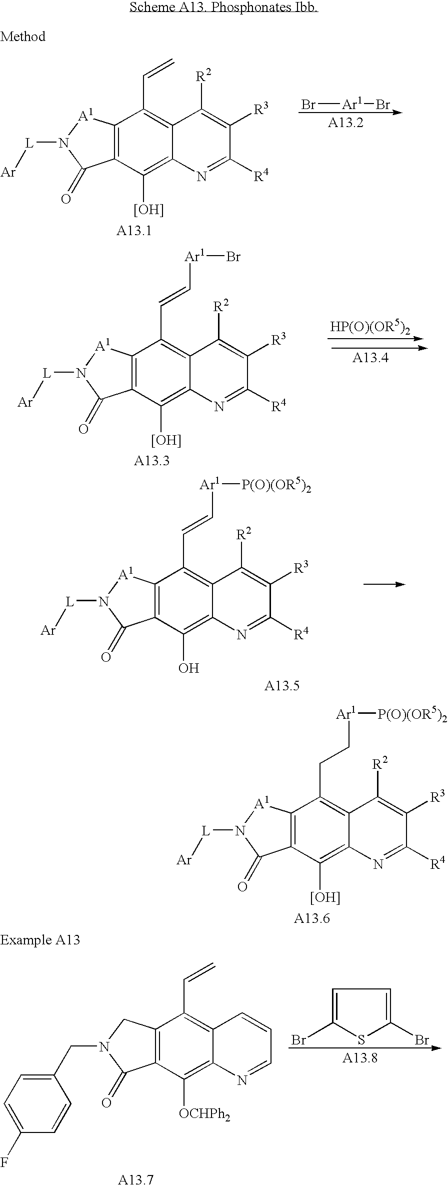 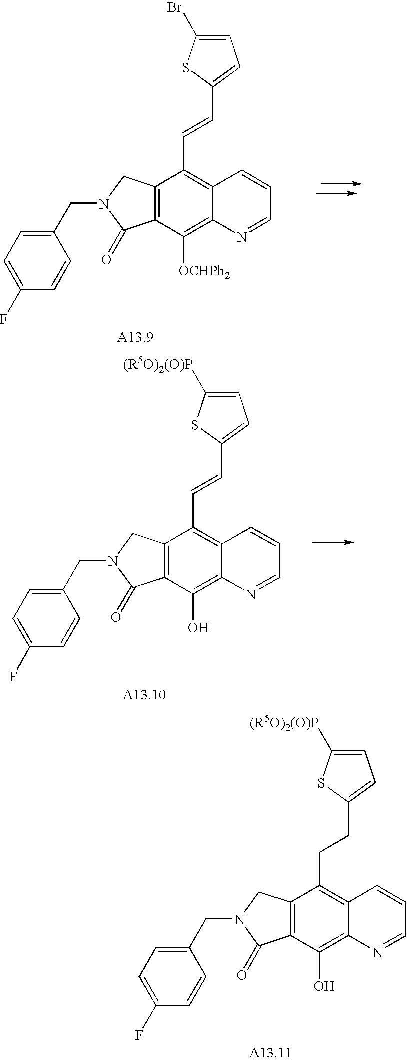 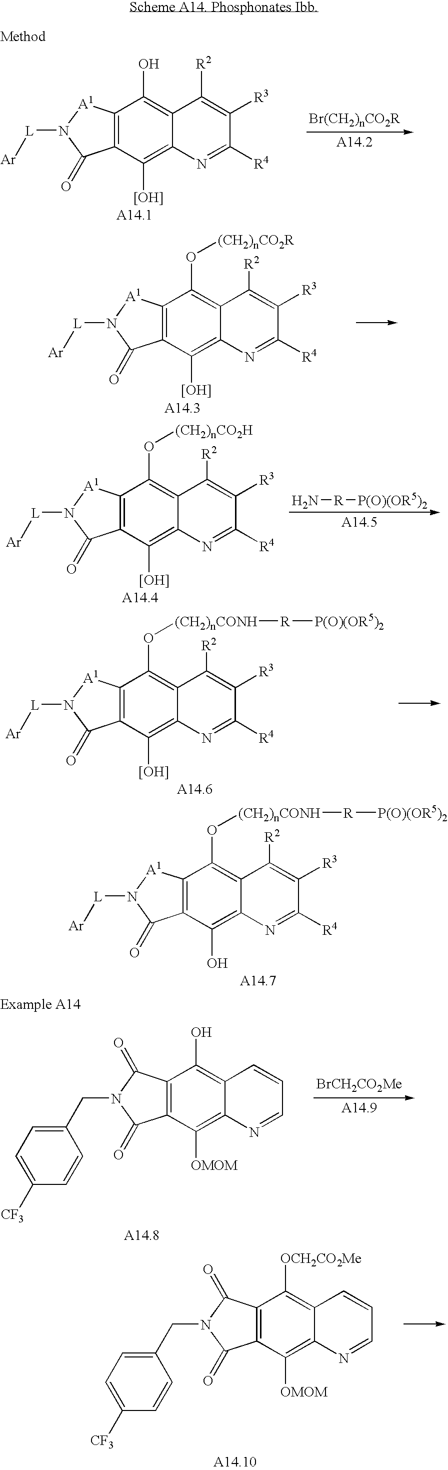 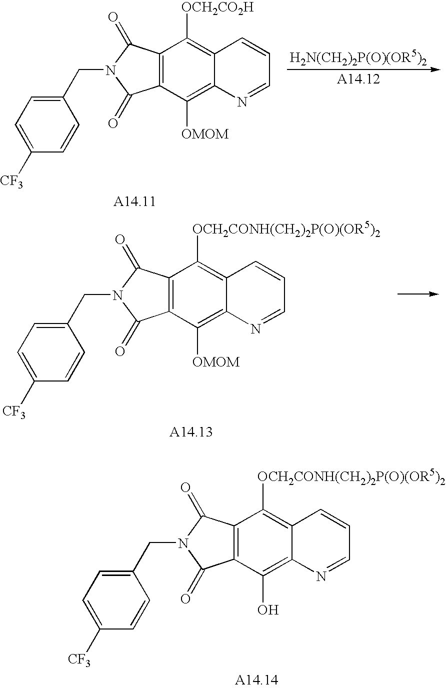 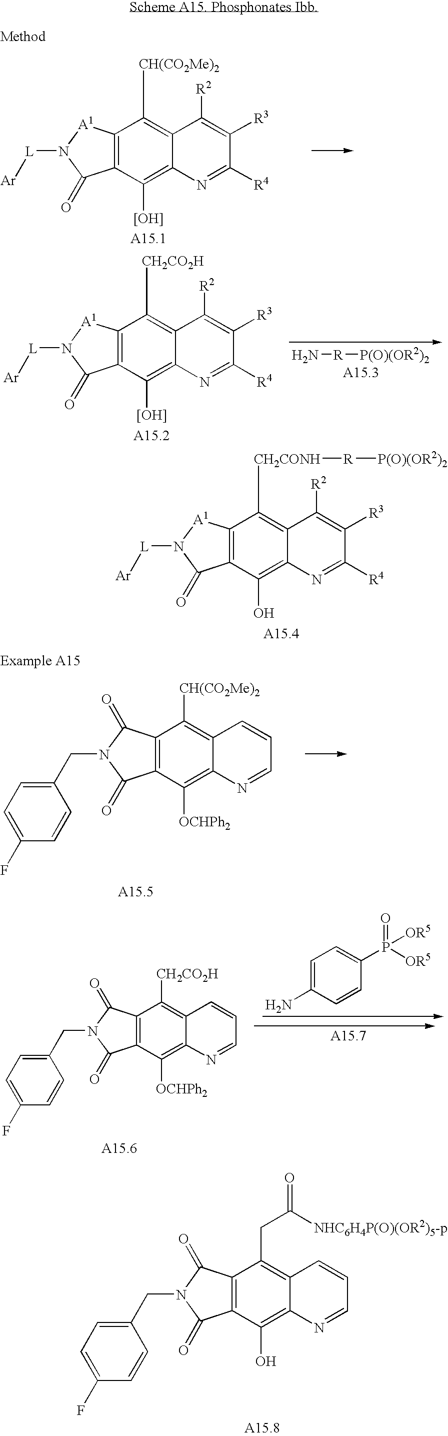 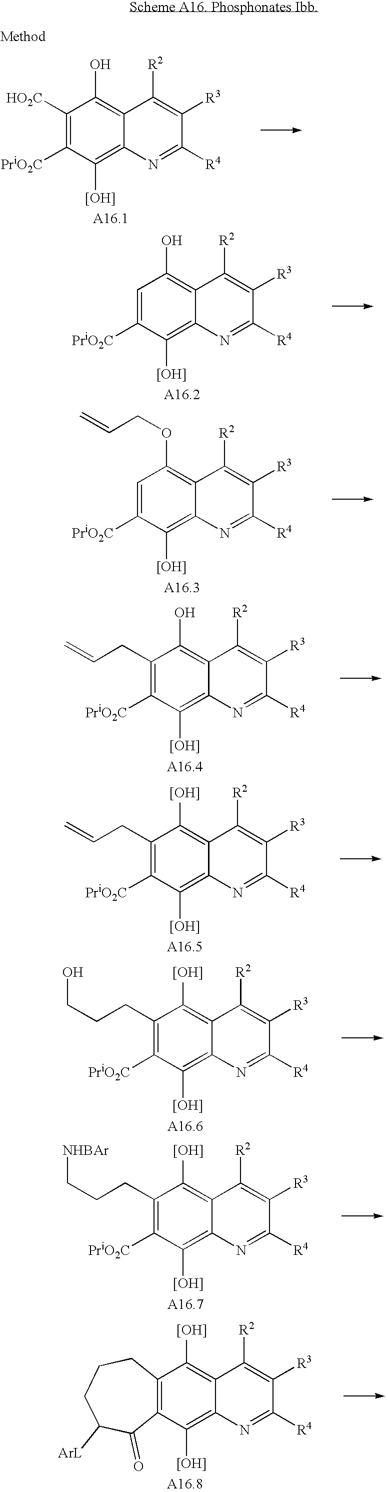 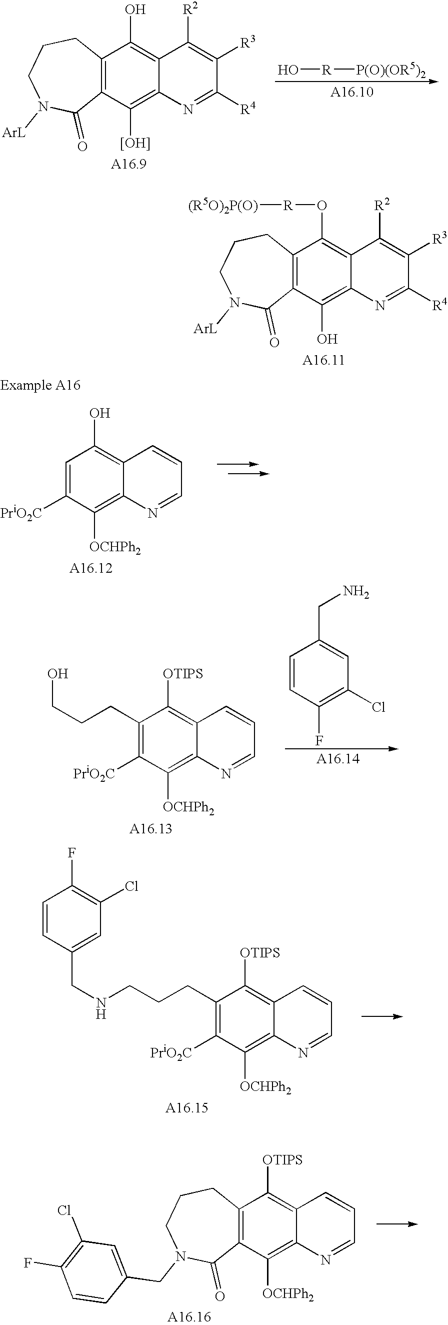 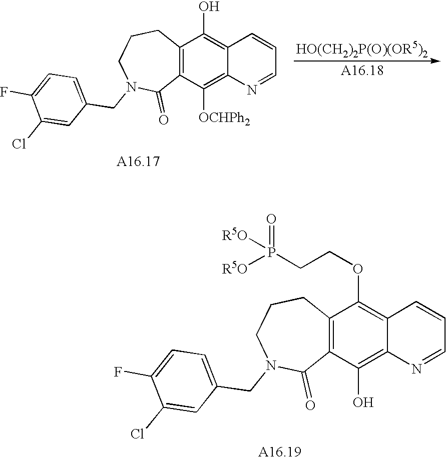
Preparation of the Intermediate Phosphonate Esters Icc.

Scheme A17 illustrates methods for the preparation of phosphonate esters of structure Icc in which the phosphonate group is attached by means of a one-carbon link, or by saturated or unsaturated multicarbon chains optionally incorporating a heteroatom. In this procedure, a 4-methyl-substituted quinoline A17.3 is prepared by means of a Doebner-von Miller condensation between an enone A17.2 and a substituted aniline A17.1. The preparation of quinolines by means of the Doebner-von Miller reaction is described in Heterocyclic Chemistry, by T. L. Gilchrist, Longman, 1992, p. 158. The reaction is performed by heating equimolar amounts of the reactants in an inert solvent such as dimethylacetamide. The bromohydroxyquinoline A17.3 is then transformed, by means of reaction sequence such as that illustrated in Scheme 8 into the protected tricyclic compound A17.4. Benzylic bromination of the latter compound, for example by reaction with N-bromosuccinimide or N-bromoacetamide in an inert solvent such as ethyl acetate at ca. 60°, then yields the bromomethyl derivative A17.5. This compound is then reacted in an Arbuzov reaction, as described above (Scheme A11), with a trialkyl phosphite to produce after deprotection the phosphonate ester A17.8.

Alternatively, the bromomethyl derivative A17.5 is reacted, using the conditions described in Scheme A12, with a dialkyl hydroxy, mercapto or amino-substituted phosphonate A17.6, in which the group R is an acyclic or cyclic saturated or unsaturated alkylene, or aryl, aralkyl or heteroaryl moiety, to give after deprotection the ether, thioether or amino product A17.7.

Alternatively, the methyl-substituted tricyclic compound A17.4 is condensed, under basic conditions, with a dialkyl formyl-substituted phosphonate A17.9. The reaction is conducted between equimolar amounts of the reactants in a polar solvent such as dioxan or dimethylformamide, in the presence of a strong base such as sodium hydride or lithium tetramethyl piperidide. The procedure affords after deprotection the unsaturated phenol A17.10. Reduction of the double bond, as described above (Scheme A13) then produces the saturated analog A17.11.

For example, benzoic acid 7-cyclopent-3-enylmethyl-4-methyl-8-oxo-7,8-dihydro-6H-pyrrolo[3,4-g]quinolin-9-yl ester A17.12 is reacted with N-bromosuccinimide in refluxing ethyl acetate to afford benzoic acid 4-bromomethyl-7-cyclopent-3-enylmethyl-8-oxo-7,8-dihydro-6H-pyrrolo[3,4-g]quinolin-9-yl ester A17.13. This compound is heated to 120° with an excess of a trialkyl phosphite to give after deprotection the phenolic phosphonate ester A17.14.

As a further example, 4-bromomethyl-7-(4-fluoro-benzyl)-9-triisopropylsilanyloxy-6,7-dihydro-pyrrolo[3,4-g]quinolin-8-one A17.15, prepared by bromination of the corresponding methyl compound is reacted with a dialkyl 2-mercaptoethyl phosphonate A17.16 (Zh. Obschei. Khim., 1973, 43, 2793) and cesium carbonate in acetonitrile, to give the thioether product A17.17. Deprotection yields the corresponding phenol A17.18.

As a further example, 7-(3-chloro-4-fluoro-benzyl)-9-methoxymethoxy-4-methyl-6,7-dihydro-pyrrolo[3,4-g]quinolin-8-one A17.19 is condensed in dioxan solution with a dialkyl formylmethyl phosphonate A17.20 (Aurora) in the presence of lithium tetramethylpiperidide to form the unsaturated product A17.21. Deprotection then yields the phenol A17.22; reduction of the double bond then gives the saturated analog A17.23.

Using the above procedures, but employing, in place of the starting materials A17.12, A17.15 and A17.19, different starting materials A17.4 or A17.5, and/or different alcohols, thiols or amines A17.6 or aldehydes A17.9, the corresponding products A17.7, A17.8, A17.10 and A17.11 are obtained. 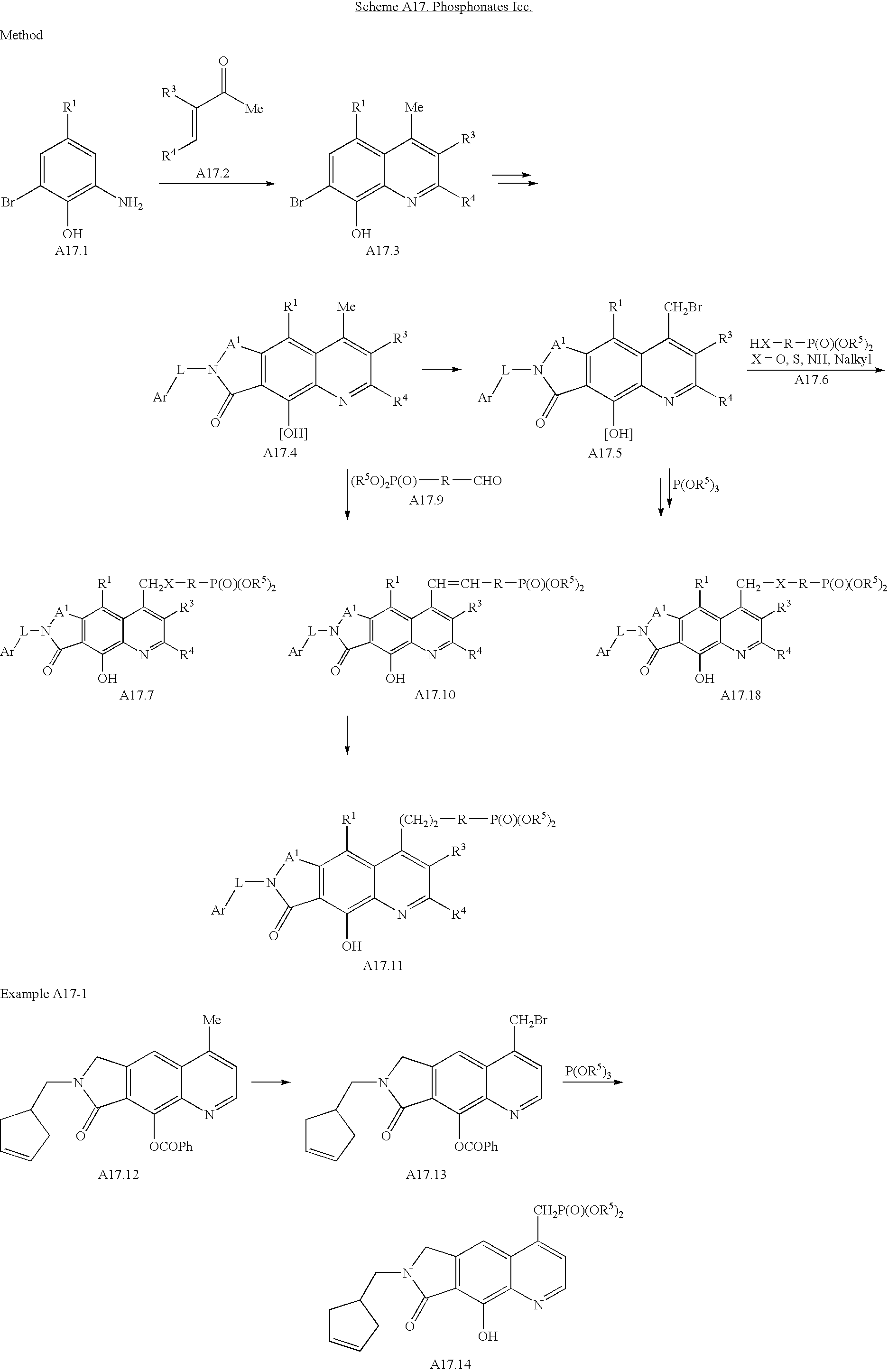 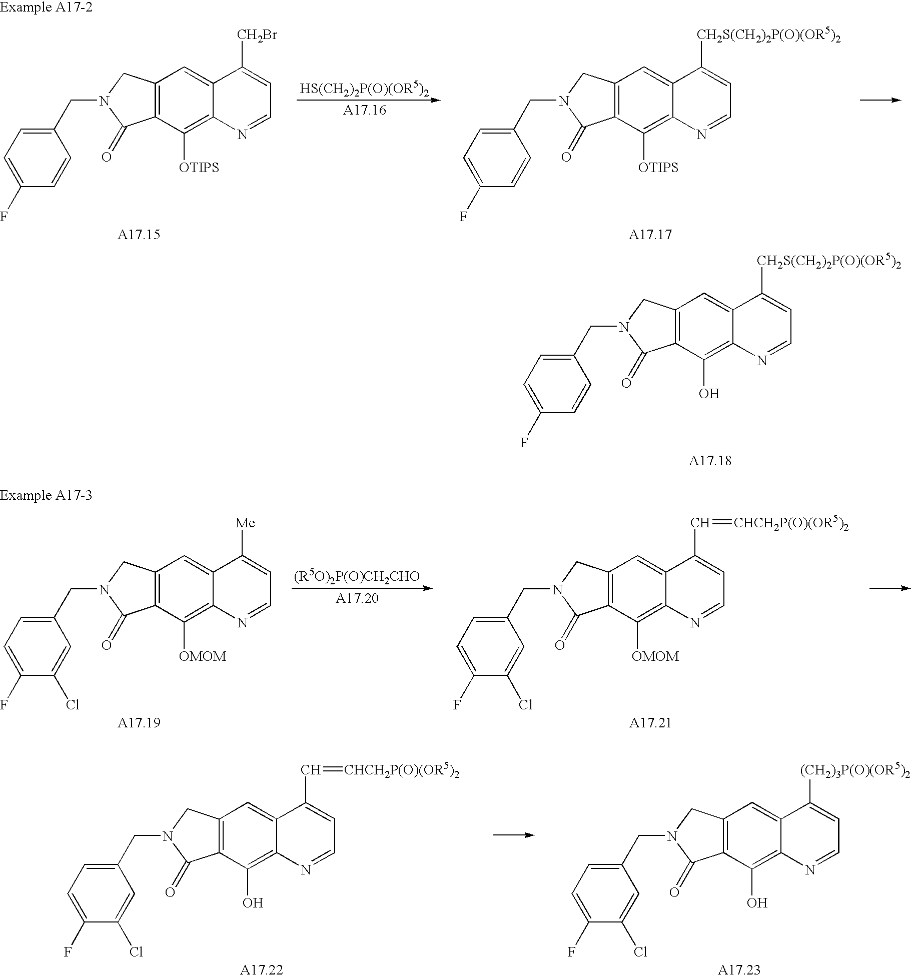
Preparation of the Intermediate Phosphonate Esters IIaa.

Schemes A18 and A19 illustrate the preparation of phosphonate esters of structure IIaa. Scheme A18 depicts the preparation of phosphonate esters of structure IIaa in which the phosphonate group is attached by means of an alkoxy, alkylthio or alkylamino group. In this procedure, an alkoxyethene triester A18.1 (JP 61289089) and a 3-aminopyridine A18.2 are reacted together, as described in JP 61289089 and GB 1509695, to produce the pyridylamino triester A18.3. The reaction is performed using equimolar amounts of the reactants at a temperature of about 150°. The product is then cyclized to afford the 1,5-naphthyridine derivative A18.4. The reaction is performed in a high-boiling solvent such as diphenyl ether at a temperature of about 250°. The diester is then converted to the anhydride, and the latter compound is transformed by reaction with the amine ArBNH₂, and protection of the phenolic hydroxyl group, into the cyclic imide A18.5. This material is then reduced, as described in Example 20, for example by the use of sodium borohydride, to afford the hydroxylactam A18.6. The latter compound is then reacted, in the presence of an acid catalyst, as described in Scheme A4, with a dialkyl hydroxy, mercapto or amino-substituted phosphonate A18.7, in which the group R is an acyclic or cyclic saturated or unsaturated alkylene, or aryl, aralkyl or heteroaryl moiety, to yield after deprotection of the phenolic hydroxy group, the ether, thioether or amino product A18.8.

For example, the triester A18.1 is reacted with 3-aminopyridine A18.9 to afford the pyridylamino triester A18.10. The product is heated in diphenyl ether at 250° to form the 1,5-naphthyridine A18.11. The latter compound is then transformed, as described above, into 7-(4-fluoro-benzyl)-6-hydroxy-9-triisopropylsilanyloxy-6,7-dihydro-pyrrolo[3,4-b][1,5]naphthyridin-8-one A18.12. The hydroxylactam is then reacted in dichloromethane solution with a dialkyl 4-hydroxybutyl phosphonate A18.13 (J. Med. Chem., 1996, 39, 949) and trifluoroacetic acid, by a similar reaction as Example 23, to generate the phosphonate product A18.14.

Using the above procedures, but employing, in place of the pyridine A18.9, different pyridines A18.2, and/or different phosphonates A18.7, the corresponding products A18.8 are obtained.

Scheme A19 depicts the preparation of phosphonate esters of structure IIaa in which the phosphonate group is attached by means of variable carbon linkage, and the nucleus is a 1,3,5,9-tetraazaanthracene. In this procedure, the 1,5-naphthyridine A18.4 is converted into the phenol-protected analog A19.1. The product is then subjected to a selective partial hydrolysis, for example by reaction with one molar equivalent of a base such as lithium hydroxide in an aqueous organic solvent mixture, to produce the carboxy ester A19.2. The product is then subjected to a Curtius rearrangement, as described in Scheme A3, to afford the amine A19.3. The product is then reductively aminated, as described in Scheme A3, by reaction with a dialkyl formyl-substituted phosphonate A19.4, in which the group R is an acyclic or cyclic saturated or unsaturated alkylene, or aryl, aralkyl or heteroaryl moiety, to give the amine A19.5. The ester group is then transformed, as described previously (Scheme A3), into the amide A19.6, by reaction with the amine ArBNH₂. The product is then cyclized to afford, after deprotection of the phenolic hydroxyl, the tricyclic product, A19.7, in which A is, for example, CO or CH₂, by reaction respectively with phosgene or an equivalent thereof, or with diiodomethane or a similar reagent.

For example, 2-amino-4-hydroxy-[1,5]naphthyridine-3-carboxylic acid methyl ester A19.8, prepared as described in Scheme A18 by the reaction between 3-aminopyridine and 1,2,2-tris-(carbomethoxy)-1-ethoxyethene, is converted, as described above, into 2-amino-4-benzyloxy-[1,5]naphthyridine-3-carboxylic acid methyl ester A19.9. The amine is then reacted in isopropanol solution with a dialkyl 3-formylphenyl phosphonate A19.10 (J. Med. Chem., 1984, 27, 654) and sodium triacetoxyborohydride, to yield the amine A19.11. The ester group of the latter compound is then transformed into the amide by reaction with 3,5-dichlorophenethylamine-trimethyl aluminum, as described previously, to afford the amide A19.12. The product is then reacted with triphosgene in pyridine solution at 80° to give the cyclized product A19.13. Deprotection then yields the phenol A19.14.

Using the above procedures, but employing, in place of the amine A19.9, different amines A19.3, and/or different formyl phosphonates A19.4, the corresponding products A19.7 are obtained. 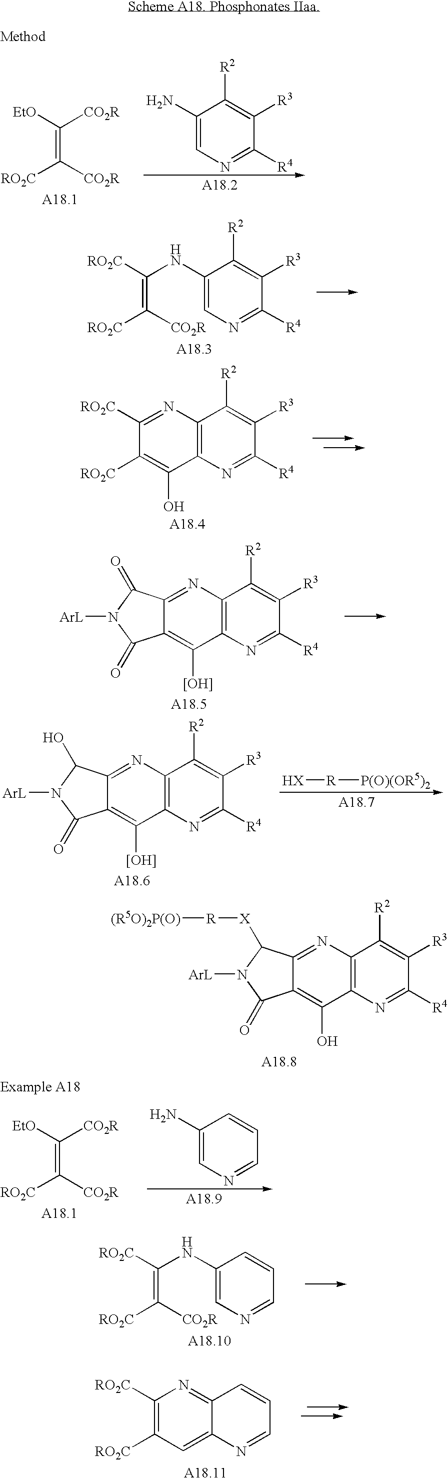 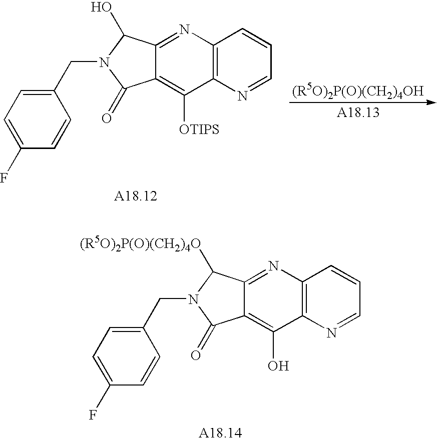 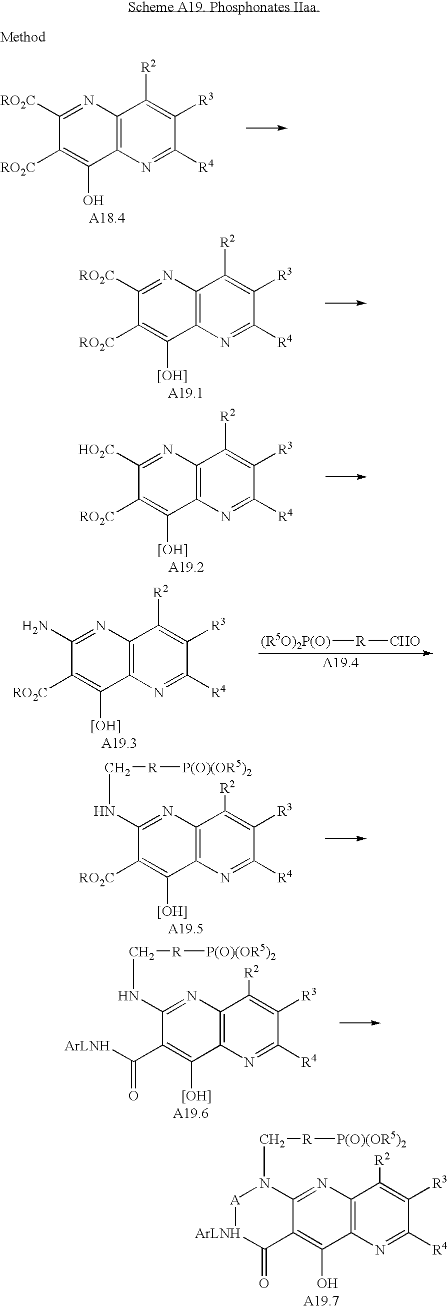 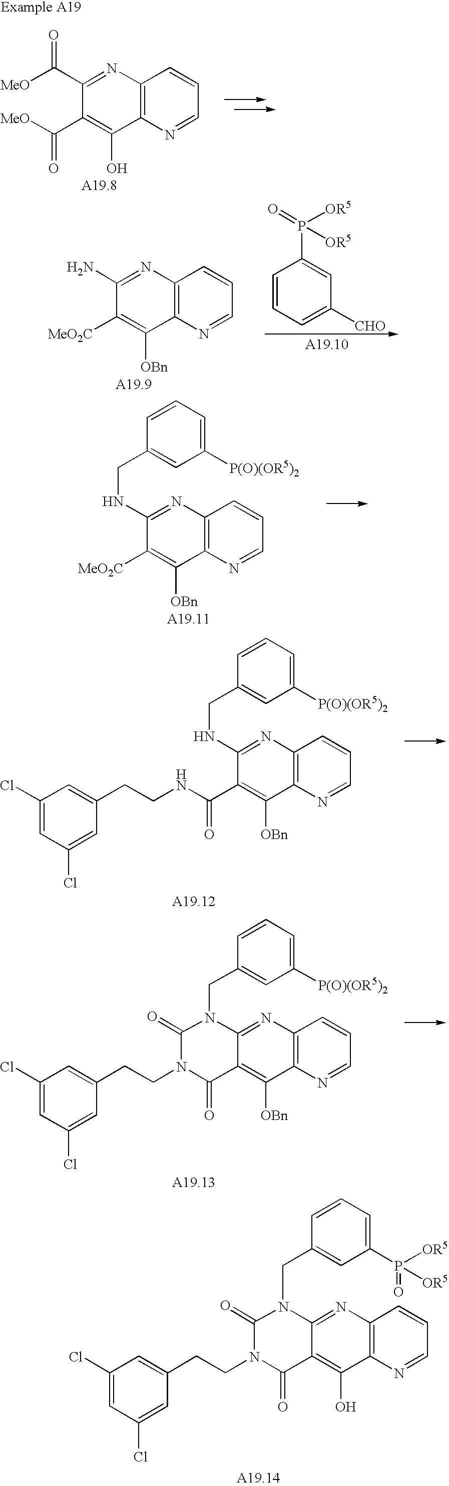
Preparation of the Intermediate Phosphonate Esters IIcc.

Scheme A20 illustrates the preparation of phosphonate esters of structure IIcc, in which the phosphonate group is attached by means of a one-carbon or multicarbon link, or by means of a heteroatom and a variable carbon linkage. In this procedure, the triester A18.1 is reacted, as described in Scheme A18, with a 3-amino-4-methylpyridine A20.1 to give the substituted pyridine product A20.2. The latter compound is then transformed, as described previously, into the methyl-substituted tricyclic compound A20.3. This compound is then subjected to benzylic bromination, for example by reaction with N-bromosuccinimide, to form the bromomethyl product A20.4. This compound is subjected to an Arbuzov reaction with a trialkyl phosphite, as described in Scheme A11, to afford after deprotection the phosphonate A20.5.

Alternatively, the bromomethyl compound A20.4 is reacted with a dialkyl phosphonate A20.6 in which X is O, S, NH or N-alkyl, and R is an acyclic or cyclic saturated or unsaturated alkylene, or aryl, aralkyl or heteroaryl moiety, using the procedures described in Scheme A17, to give, after deprotection of the phenolic hydroxyl, the ether, thioether or amine products A20.7.

Alternatively, the methyl compound A20.3 is subjected, as described in Scheme A17, to a base-catalyzed condensation reaction with a dialkyl formyl-substituted phosphonate A20.8, in which R is an acyclic or cyclic saturated or unsaturated alkylene, or aryl, aralkyl or heteroaryl moiety, to generate after deprotection of the phenolic hydroxyl, the unsaturated product A20.9. The double bond is then reduced, as described in Scheme A17, to afford the saturated analog A20.10.

For example, condensation between the triester A18.1 and 3-amino-4-methylpyridine A20.11 gives the pyridine product A20.12. The compound is then transformed, as described above, into 7-[1-(4-fluoro-phenyl)-1-methyl-ethyl]-4-methyl-9-triisopropylsilanyloxy-pyrrolo[3,4-b][1,5]naphthyridine-6,8-dione A20.13. The latter compound is then reacted with a dialkyl formylethyl phosphonate A20.14 (Zh. Obschei. Khim., 1987, 57, 2793) and lithium tetramethylpiperidide in tetrahydrofuran to afford after deprotection the unsaturated product A20.15. The product is then reduced with diimide, as described above, (Scheme A13) to yield the saturated analog A20.16.

As a further example, 7-[1-(4-fluoro-phenyl)-cyclopropyl]-4-methyl-9-triisopropylsilanyloxy-pyrrolo[3,4-b][1,5]naphthyridine-6,8-dione A20.17, prepared according to the procedures described above, is reacted with N-bromosuccinimide in refluxing ethyl acetate to give 4-bromomethyl-7-[1-(4-fluoro-phenyl)-cyclopropyl]-9-triisopropylsilanyloxy-pyrrolo[3,4-b][1,5]naphthyridine-6,8-dione A20.18. The product is then heated at 120° with excess of a trialkyl phosphite to give after deprotection the phosphonate A20.19.

As a further example, 4-bromomethyl-7-(3-chloro-4-fluoro-benzyl)-9-triisopropylsilanyloxy-pyrrolo[3,4-b][1,5]naphthyridine-6,8-dione A20.20, prepared according to the procedures described above, is reacted in dimethylformamide solution with a dialkyl methylaminomethyl phosphonate A20.21 (AsInEx) and potassium carbonate, to afford after deprotection the displacement product A20.22.

Using the above procedures, but employing, in place of the starting materials A20.13, A20.17 and A20.20, different starting materials A20.3 or A20.4, and/or different alcohols, thiols or amines A20.6 or aldehydes A20.8, the corresponding products A20.5, A20.7, A20.9 and A20.10 are obtained. 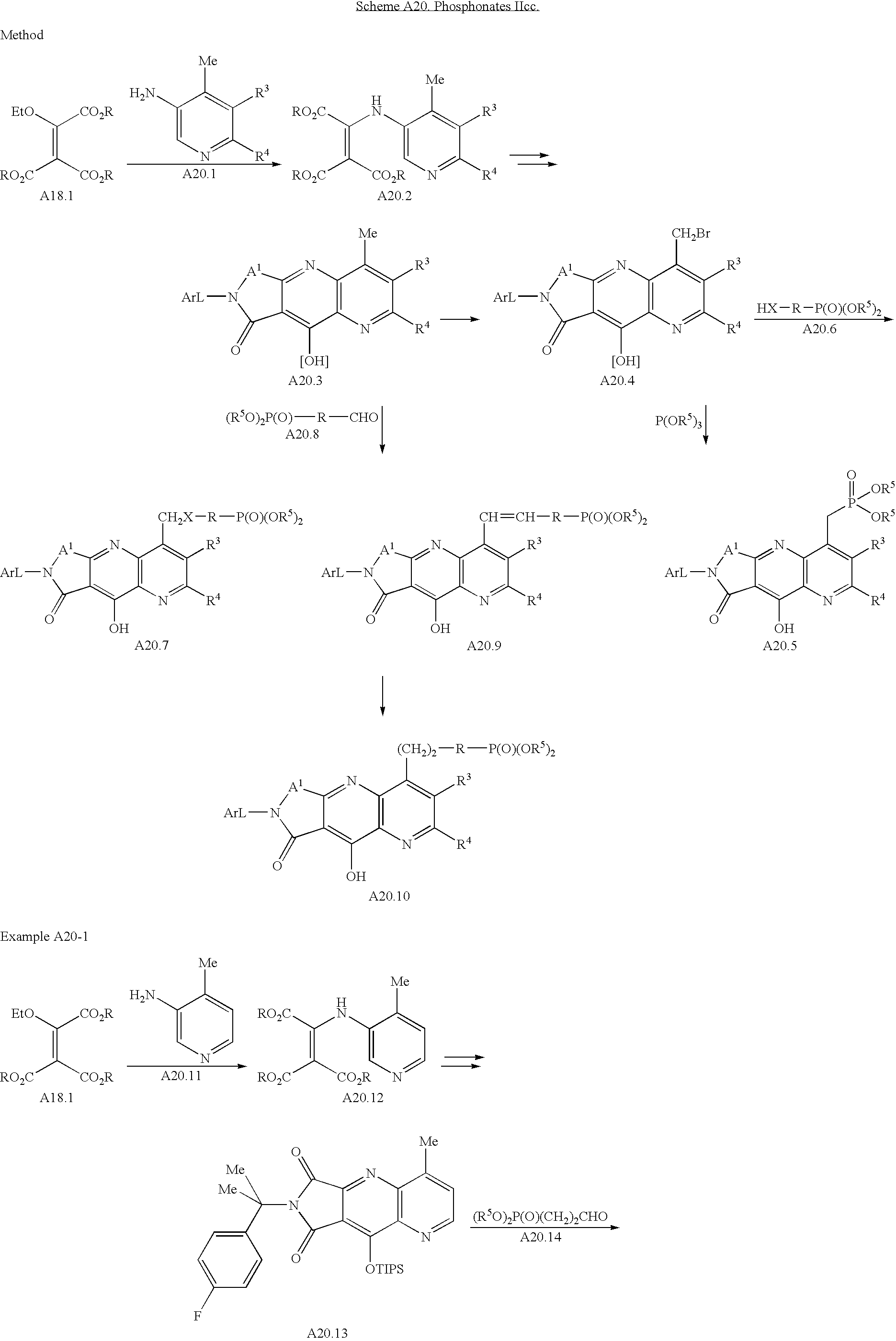 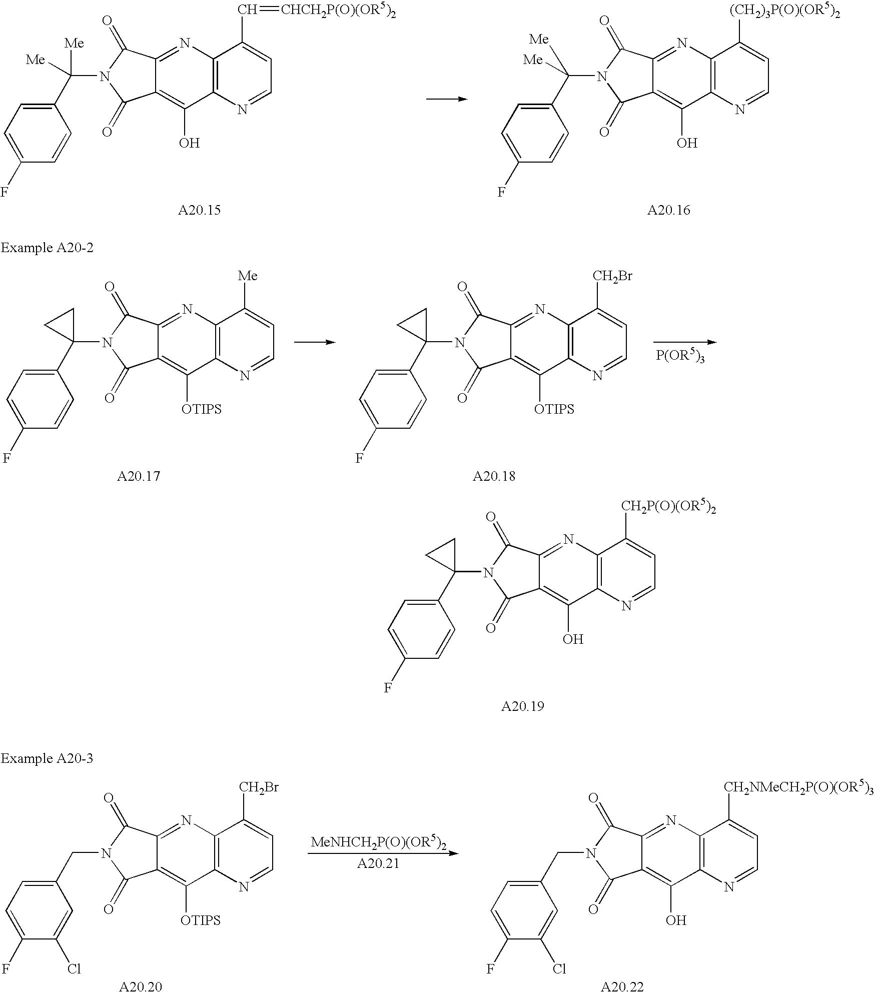
Preparation of the Intermediate Phosphonate Esters IIIaa.

Scheme A21 illustrates methods for the preparation of phosphonates of structure IIIaa in which the phosphonate group is attached by means of a heteroatom and a variable carbon link. In this sequence, a carbomethoxymethyl derivative of the amine ArBNH₂, A21.1 is coupled with the 1,6-naphthyridine carboxylic acid A21.2, prepared as described in WO 0230930, using the methods described previously, to prepare the amide A21.3. Bromination, for example using N-bromosuccinimide, yields the 5-bromo derivative A21.4. Protection of the phenolic hydroxyl group, followed by displacement of the bromine with a hydrazine or hydroxylamine nucleophile, as described for example in Example 69, affords the 5-imino derivative A21.5 in which X is NH₂ or OH. Lactam formation, for example by the use of potassium tert. butoxide in refluxing xylene, or by the use of trimethylaluminum, then gives the tricyclic product A21.6, which upon protection of the X substituent gives the product A21.7. Reduction of this material, for example by treatment with sodium borohydride, for example as in Example 20, then gives the aminol A21.8. The latter compound is reacted with a dialkyl hydroxy, mercapto, or amino-substituted phosphonate A21.9, in which the group R is an acyclic or cyclic saturated or unsaturated alkylene, or aryl, aralkyl or heteroaryl moiety, in the presence of an acid such as trifluoroacetic acid, as described in Scheme A4, to yield the ether, thioether or amine product A21.10. Deprotection then gives the phenol A21.11.

For example, (4-fluoro-benzylamino)-acetic acid methyl ester A21.12 is coupled in tetrahydrofuran solution with one molar equivalent of 8-hydroxy-[1,6]naphthyridine-7-carboxylic acid A21.13, (WO 0230930) in the presence of diisopropyl carbodiimide, to form [(4-fluoro-benzyl)-(8-hydroxy-[1,6]naphthyridine-7-carbonyl)-amino]-acetic acid methyl ester A21.14. The latter compound is then transformed, by bromination, displacement and cyclization, as described above into the tricyclic product, 9-benzyloxy-7-(4-fluoro-benzyl)-10-hydrazono-6,7-dihydro-10H-1,7,10a-triaza-anthracene-5,8-dione A21.15. The hydrazono compound is then converted into the N,N-dibenzyl derivative A21.16. The conversion of amines into dibenzylamines, for example by treatment with benzyl bromide in a polar solvent such as acetonitrile or aqueous ethanol, in the presence of a base such as triethylamine or sodium carbonate, is described in Protective Groups in Organic Synthesis, by T. W. Greene and P. G. M Wuts, Wiley, Second Edition 1990, p. 364. The tribenzylated compound is then reduced with a limited amount of sodium borohydride in isopropanol to afford the aminal A21.17. This compound is reacted with a dialkyl 2-mercaptoethyl phosphonate A21.18 (Zh. Obschei. Khim., 1973, 43, 2364), and trifluoroacetic acid in dichloromethane, to give the thioether A21.19. Debenzylation, for example by the use of 5% palladium on carbon in the presence of ammonium formate, as described in Tet. Lett., 28, 515, 1987, then affords the hydrazono phenol A21.20.

Using the above procedures, but employing, in place of the amide A21.14, different amides A21.3, and/or different phosphonates A21.9, the corresponding products A21.11 are obtained. 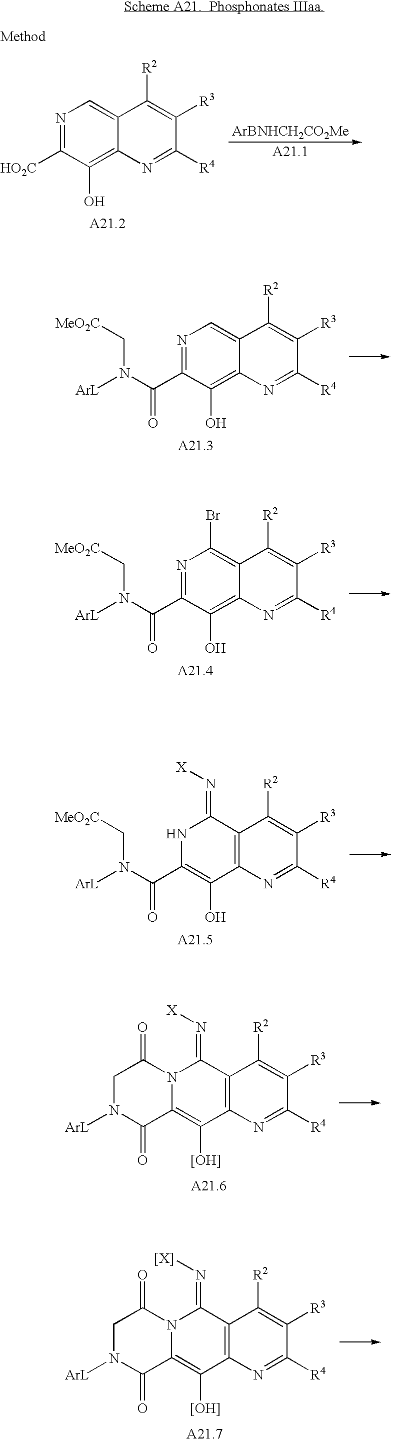 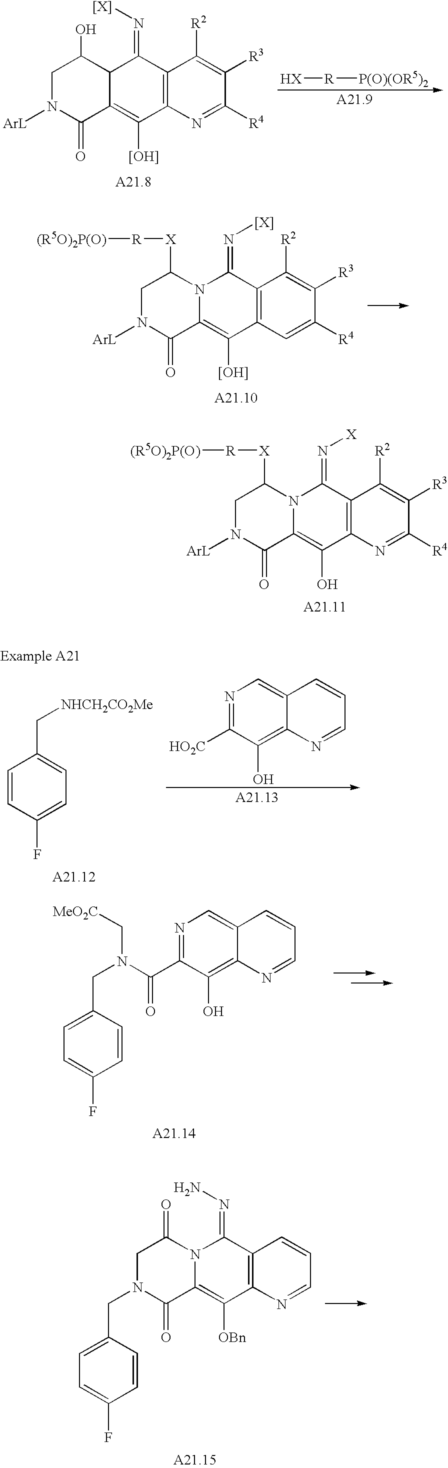 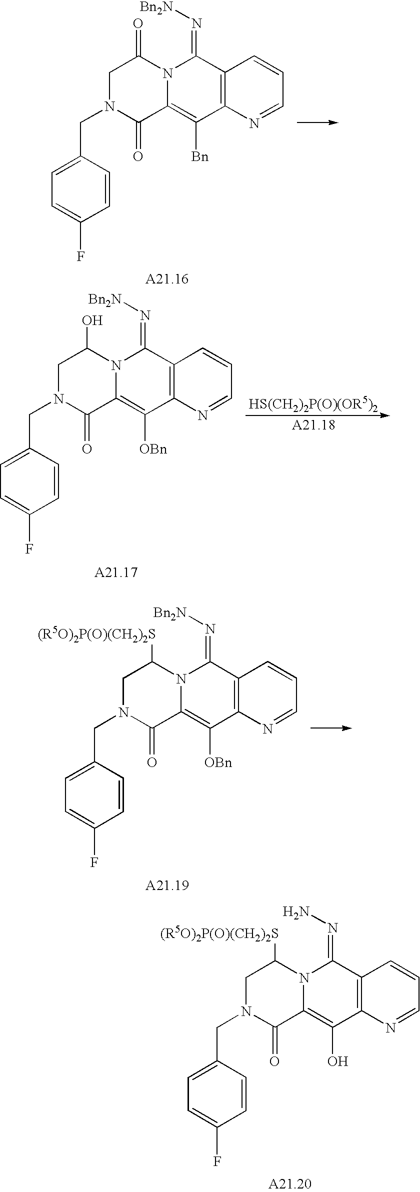
Preparation of the Intermediate Phosphonate Esters IIIbb.

Schemes A22-A24 illustrate methods for the preparation of phosphonate esters of structure IIIbb.

Scheme A22 illustrates methods for the preparation of phosphonates of structure IIIbb in which the phosphonate group is attached by means of a variable carbon linkage. In this sequence, the naphthyridine carboxylic acid A21.2 is coupled, as described previously, with the amine derivative A22.1, following a procedure similar to Example 28, to form the amide A22.2. Bromination, as described above, yields the 5-bromo derivative A22.3, which upon protection of the phenolic hydroxyl yields the compound A22.4. Displacement of the bromine, by reaction with a dialkyl amino-substituted phosphonate A22.5, in which the group R is an acyclic or cyclic saturated or unsaturated alkylene, or aryl, aralkyl or heteroaryl moiety, affords the amine A22.6. The reaction is performed in a polar organic solvent such as dimethylformamide in the presence of a base such as potassium carbonate. Deprotection of the alcoholic hydroxyl group affords the alcohol A22.7, which upon activation and cyclization, for example as described in Scheme 11 then gives the tricyclic product A22.8, which upon deprotection affords the phenol A22.9.

For example, acetic acid 5-bromo-7-[(4-fluoro-benzyl)-propyl-carbamoyl]-[1,6]naphthyridin-8-yl ester A22.10, is reacted with one molar equivalent of a dialkyl aminopropyl phosphonate A22.11, (Acros) to yield the amine A22.12. Deprotection and activation of the alcoholic hydroxyl group, for example by conversion to the mesylate, followed by cyclization under basic conditions, and deprotection of the phenolic hydroxyl group, then affords the enol A22.13.

Using the above procedures, but employing, in place of the bromide A22.10, different bromides A22.4, and/or different aminophosphonates A22.5, the corresponding products A22.9 are obtained.

Scheme A23 illustrates methods for the preparation of phosphonates of structure IIIbb in which the phosphonate group is attached by means of a nitrogen and a variable carbon linkage. In this sequence, a tricyclic imine A23.1 (Scheme 12) is reacted with a dialkyl bromoalkyl phosphonate A23.2 to give the alkylated product A23.3. The reaction is performed in a polar organic solvent such as acetonitrile or dimethylsulfoxide, in the presence of a base such as diisopropylethylamine or 2,6-lutidine.

Alternatively, the imine A23.1 is converted into a hydrazone A23.5 by reaction with a dialkyl formyl-substituted phosphonate A23.4 in which the group R is an acyclic or cyclic saturated or unsaturated alkylene, or aryl, aralkyl or heteroaryl moiety. The hydrazone is prepared by the reaction of equimolar amounts of the reactants in a polar organic solvent such as ethanol, optionally in the presence of a catalytic amount of an acid such as acetic acid. Optionally, the hydrazone product A23.5 is reduced, for example by treatment with sodium borohydride, to give the dihydro derivative A23.6.

For example, acetic acid 7-(4-fluoro-benzyl)-10-hydrazono-8-oxo-6,7,8,10-tetrahydro-5H-1,7,10a-triaza-anthracen-9-yl ester A23.7 (Scheme 12) is reacted at 60° in dimethylformamide solution containing potassium carbonate with one molar equivalent of a dialkyl 2-bromoethyl phosphonate A23.8 (Aldrich), to prepare the alkylated product which upon deprotection yields the enol A23.9.

As a further example, the hydrazone A23.7 is reacted in ethanol solution at ambient temperature with one molar equivalent of a dialkyl 2-formylphenyl phosphonate A23.10 (Epsilon) to give the hydrazone product A23.11. Reduction of the double bond, by treatment with sodium cyanoborohydride in isopropanol, followed by deprotection, affords the enol product A23.12.

Using the above procedures, but employing, in place of the hydrazone A23.7, different hydrazones A23.1, and/or different bromophosphonates A23.2, or formyl phosphonates A23.4 the corresponding products A23.3, A23.5 and A23.6 are obtained.

Scheme A24 illustrates methods for the preparation of phosphonates of structure IIIbb in which the phosphonate group is attached by means of a hydroxyimino linkage. In this sequence, a tricyclic oxime A24.1 (Scheme 14) is reacted with a dialkyl bromo-substituted phosphonate A24.2 in which the group R is an acyclic or cyclic saturated or unsaturated alkylene, or aryl, aralkyl or heteroaryl moiety. The reaction is performed in a polar organic solvent in the presence of a base such as sodium hydride or lithium hexamethyldisilazide. Deprotection then yields the enol A24.4.

For example, acetic acid 7-(4-fluoro-benzyl)-10-hydroxyimino-8-oxo-6,7,8,10-tetrahydro-5H-1,7,10a-triaza-anthracen-9-yl ester A24.5 (Scheme 14) is reacted in dimethylformamide solution with one molar equivalent of sodium hydride, followed by the addition of one molar equivalent of a dialkyl 4-(bromomethyl)phenyl phosphonate A24.6 (Tet., 1998, 54, 9341) to afford after deprotection the iminoether A24.7.

Using the above procedures, but employing, in place of the oxime A24.5, different oximes A24.1, and/or different phosphonates A24.2, the corresponding products A24.4 are obtained. 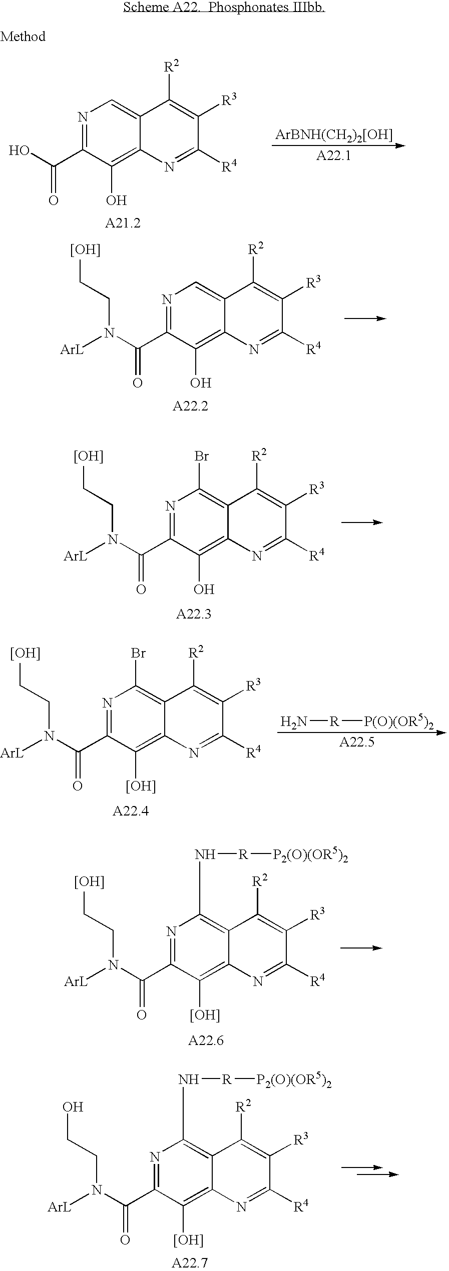 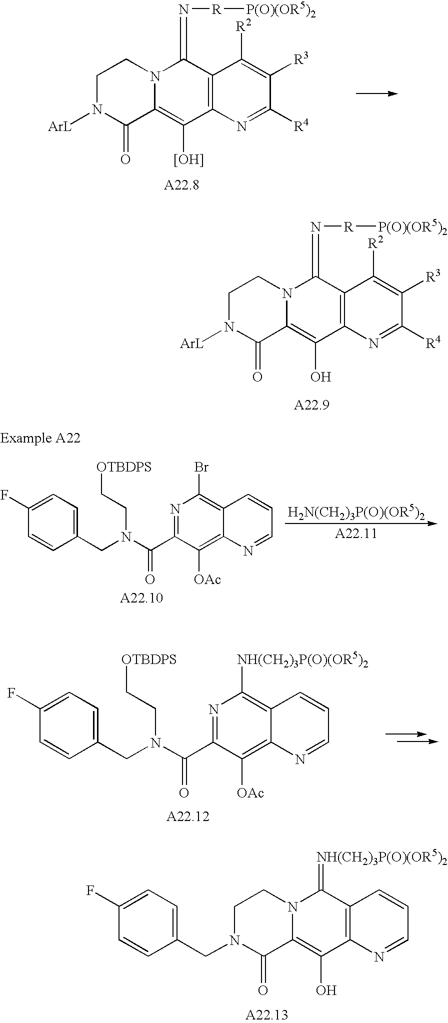 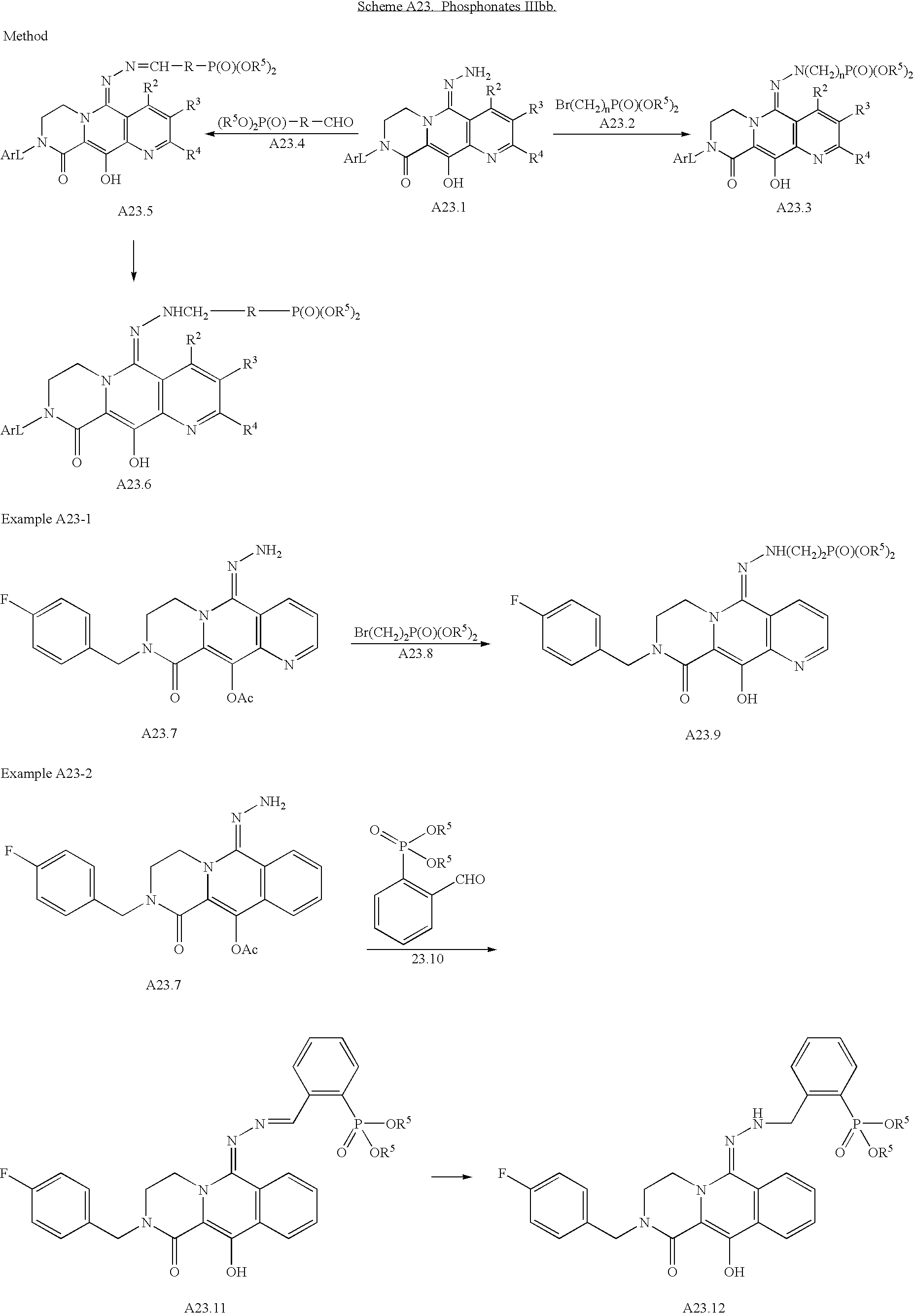 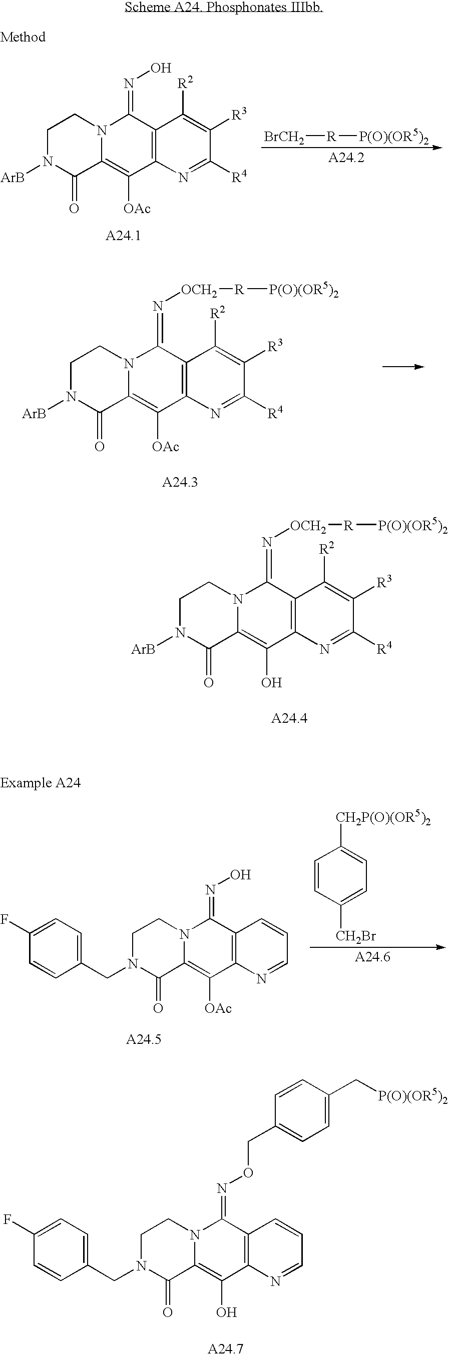
Preparation of the Intermediate Phosphonate Esters IIIcc.

Scheme A25 illustrates methods for the preparation of phosphonates of structure IIIcc. The conversion of pyridine-2,3-dicarboxylic anhydride (A25.1, R=H) into the naphthyridine A25.2, R=H, is described in WO 0255079. Using the same procedure, 4-methylpyridine-2,3-dicarboxylic anhydride A25.1, R=Me, (J. Org. Chem., 1961, 26, 808) is converted into the naphthyridine A25.2, R=Me. This compound is then transformed, as described in Scheme 12, into the imine A25.3. Protection of the hydroxyl and amino groups then furnishes the derivative A25.4. The product is then condensed under basic conditions, as described in Scheme A20, with a dialkyl formyl-substituted phosphonate A25.5, in which the group R is an acyclic or cyclic saturated or unsaturated alkylene, or aryl, aralkyl or heteroaryl moiety. After deprotection, the product A25.6 is optionally reduced, as described in Scheme A20, to give the saturated analog A25.17.

Alternatively, the methyl-substituted tricycle A25.4 is brominated, for example by reaction with N-bromosuccinimide, to give the bromomethyl product A25.7. The compound is then subjected to a Arbuzov reaction with a trialkyl phosphite, to yield after deprotection the phosphonate A25.8.

Alternatively, the bromomethyl compound A25.7 is reacted, as described previously (Scheme A20) with a dialkyl hydroxy, mercapto or amino-substituted phosphonate A25.18, in which the group R is an acyclic or cyclic saturated or unsaturated alkylene, or aryl, aralkyl or heteroaryl moiety, to give after deprotection the ether, thioether or amine product A25.9.

For example, acetic acid 7-[2-(4-fluoro-phenyl)-ethyl]-10-hydrazono-4-methyl-8-oxo-6,7,8,10-tetrahydro-5H-1,7,10a-triaza-anthracen-9-yl ester A25.10, prepared according to the procedures described above, is converted into the phthalimido derivative by reaction with one molar equivalent of phthalic anhydride, as described in J. Org. Chem., 43, 2320, 1978. The protected product is then reacted with N-bromosuccinimide in hexachloroethane to give the bromomethyl derivative A25.12. This compound is heated to 120° with an excess of a trialkyl phosphite to produce the phosphonate A25.13. Deprotection, for example by reaction with ethanolic hydrazine, as described in J. Org. Chem., 43, 2320, 1978, then affords the phosphonate A25.14.

As a further example, the phthalimido-protected methyl-substituted tricycle A25.11 is reacted in dioxan solution with a dialkyl formylphosphonate A25.12 (Tet., 1994, 50, 10277) and lithium tetramethyl piperidide, to yield, after removal of the protecting groups, the unsaturated phosphonate A25.13. Reduction of the double bond then gives the saturated analog A25.14.

As a further example, the bromomethyl derivative A25.12 is reacted in acetonitrile solution with one molar equivalent of a dialkyl 2-mercaptopropyl phosphonate A25.15 (WO 007101) and diisopropylethylamine, to produce after deprotection the phosphonate A25.16.

Using the above procedures, but employing, in place of the starting materials A25.10, A25.11 or A25.12, different starting materials A25.4, and A25.7, and/or different aldehydes A25.5 or alcohols, thiols or amines A25.18, the corresponding products A25.6, A25.8, A25.9 and A25.17 are obtained. 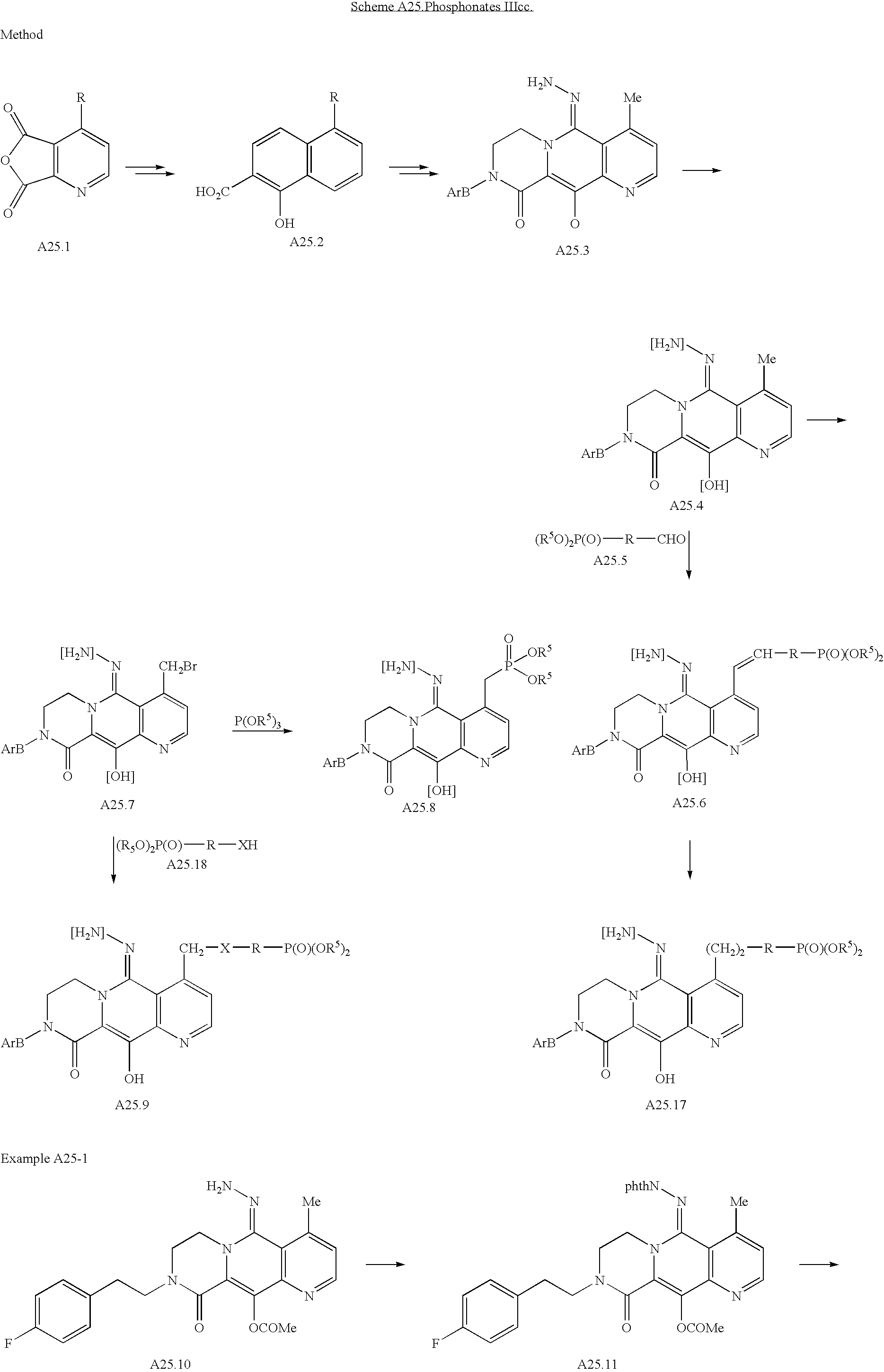 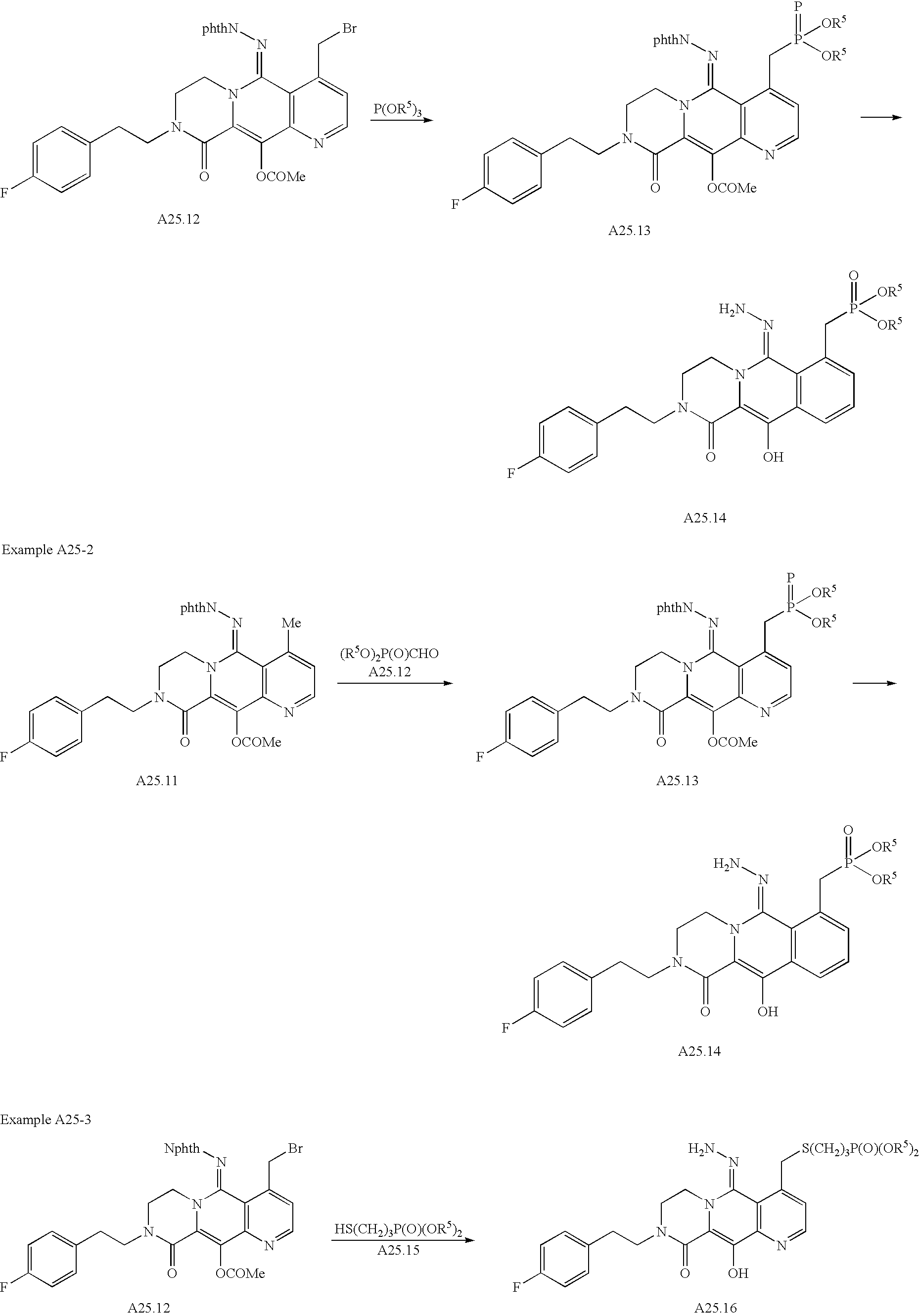
Preparation of the Intermediate Phosphonate Esters IVaa.

Schemes A29 and A30 illustrates the preparation of phosphonate esters of structure IVaa.

Scheme A29 illustrates the preparation of compounds in which phosphonate is attached by means of an ether, thioether of amine linkage. In this procedure, a substituted succinimide A29.1 is condensed, as described in Scheme 1 and Example 2, with a heterocyclic diester A29.2 to afford after protection the tricyclic product A29.3. Reduction with sodium borohydride then yields the aminal A29.4, which upon acid-catalyzed reaction with a dialkyl hydroxy, mercapto or amino-substituted phosphonate A29.5, in which the group R is an acyclic or cyclic saturated or unsaturated alkylene, or aryl, aralkyl or heteroaryl moiety, to give after deprotection the ether, thioether or amine products A29.6.

For example, 1-[2-(4-fluoro-phenyl)-cyclopropyl]-pyrrolidine-2,5-dione A29.7, prepared from 4-fluorophenylcyclopropylamine (J. Med. Chem., 1996, 39, 1485) and succinic anhydride, is reacted with 4,5-dicarbomethoxyisoxazole A29.8 (Chem. Ber., 97, 1414, 1964) to afford after protection 6-[2-(4-fluoro-phenyl)-cyclopropyl]-4,8-bis-methoxymethoxy-oxazolo[4,5-f]isoindole-5,7-dione A29.9. Reduction with sodium borohydride then gives the aminal A29.10, which upon reaction with a dialkyl 3-mercaptopropyl phosphonate A29.11 (WO 0077101) and trifluoroacetic acid in dichloromethane yields the phosphonate thioether A29.12.

Using the above procedures, but employing, in place of the starting materials A29.7 and A29.8, different starting materials A29.1 and A29.2, and/or different phosphonates A29.5, the corresponding products A29.6 are obtained.

Preparation of the Intermediate Phosphonate Esters IVaa.

Scheme A30 illustrates the preparation of phosphonate esters of structure IVaa in which the phosphonate is attached by means of a variable carbon linkage. In this procedure, dimethyl succinate A30.1 is condensed, under base catalysis, for example using the procedure described on Scheme 1 and Example 2 with a heterocyclic diester A30.2, to yield after protection of the phenolic hydroxyl groups, the diester A30.3. Partial basic hydrolysis, for example by reaction with one molar equivalent of lithium hydroxide in aqueous dimethoxyethane, then affords the monoacid A30.4. The carboxylic acid is homologated to produce the corresponding acetic acid A30.5. The transformation is effected by means of the Arndt Eistert reaction. In this procedure, which is described in Advanced Organic Chemistry, Part B, by F. A. Carey and R. J. Sundberg, Plenum, 2001, p. 641, and in Advanced Organic Chemistry, By J. Marsh, McGraw Hill, 1968, p. 809, the carboxylic acid is converted into the acid chloride, which is reacted with diazomethane to give the corresponding diazoketone. Silver-catalyzed Wolff rearrangement of the diazoketone in an alcoholic solvent then yields the acetic acid ester, which upon hydrolyis yields the acetic acid A30.5. This material is coupled with the amine A30.6 to give the amide A30.7. Base-catalyzed thermal cyclization of the latter compound, for example by refluxing in xylene with sodium methoxide, then gives the cyclized product A30.8. The latter compound is then alkylated, as described above, (Scheme A10) with a dialkyl bromo-substituted phosphonate A30.9, in which the group R is an acyclic or cyclic saturated or unsaturated alkylene, or aryl, aralkyl or heteroaryl moiety, to afford after deprotection the phosphonate A30.10.

For example, condensation between dimethyl succinate and methyl 1-methylimidazole-4,5-dicarboxylate A30.11 (Eqypt. J. Chem., 1985, 28, 139) yields, after protection of the phenolic hydroxyl groups, 4,7-bis-methoxymethoxy-1-methyl-1H-benzoimidazole-5,6-dicarboxylic acid dimethyl ester A30.12. Partial hydrolysis then gives the monocarboxylic acid A30.13, and this compound is subjected to Arndt Eistert homologation to give the corresponding acetic acid A30.14. The carboxylic acid is coupled, in the presence of dicyclohexyl carbodiimide, with cyclohexylmethylamine A30.15 to give the amide A30.16. Cyclization is effected as described above to prepare 6-cyclohexylmethyl-4,9-bis-methoxymethoxy-1-methyl-1,5,6,8-tetrahydro-1,3,6-triaza-cyclopenta[b]naphthalen-7-one A30.17. The product is then reacted in dioxan solution with a dialkyl bromoethyl phosphonate A30.18 (Aldrich) and lithium hexamethyldisilazide, to give after deprotection the phosphonate A30.19.

Using the above procedures, but employing, in place of the starting materials A30.1 and A30.11, different starting materials A30.1 and A30.2, and/or different phosphonates A30.9, the corresponding products A30.10 are obtained. 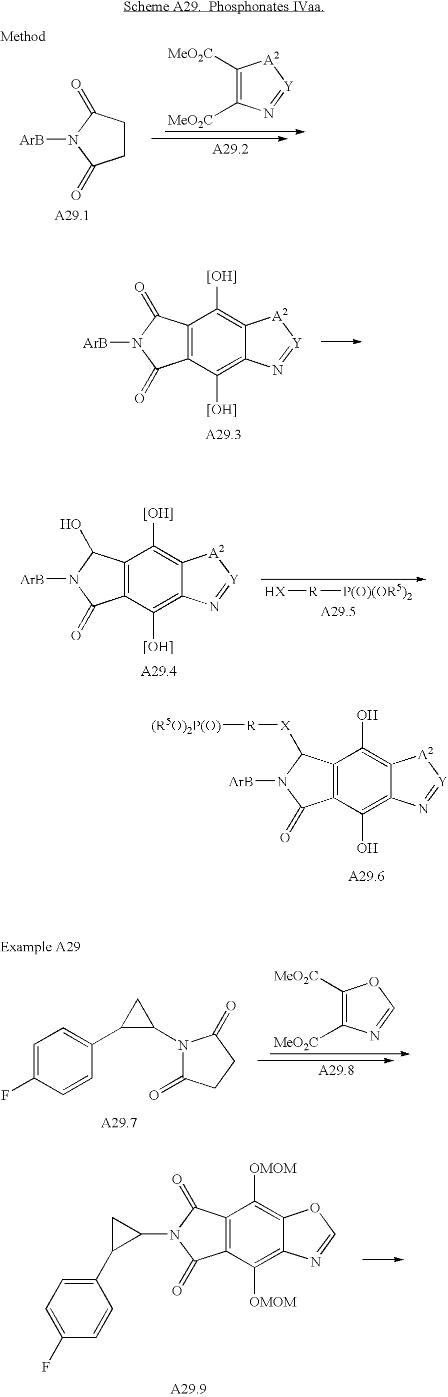 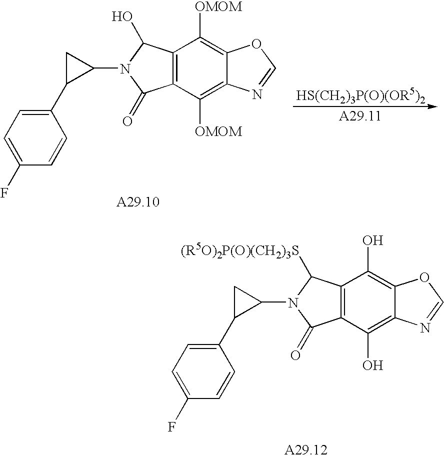 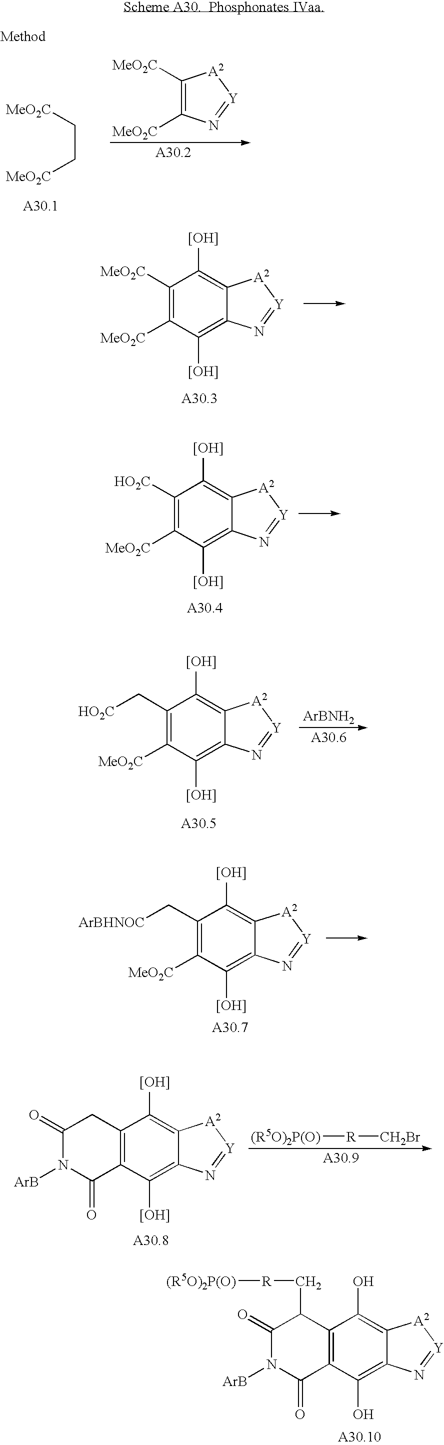 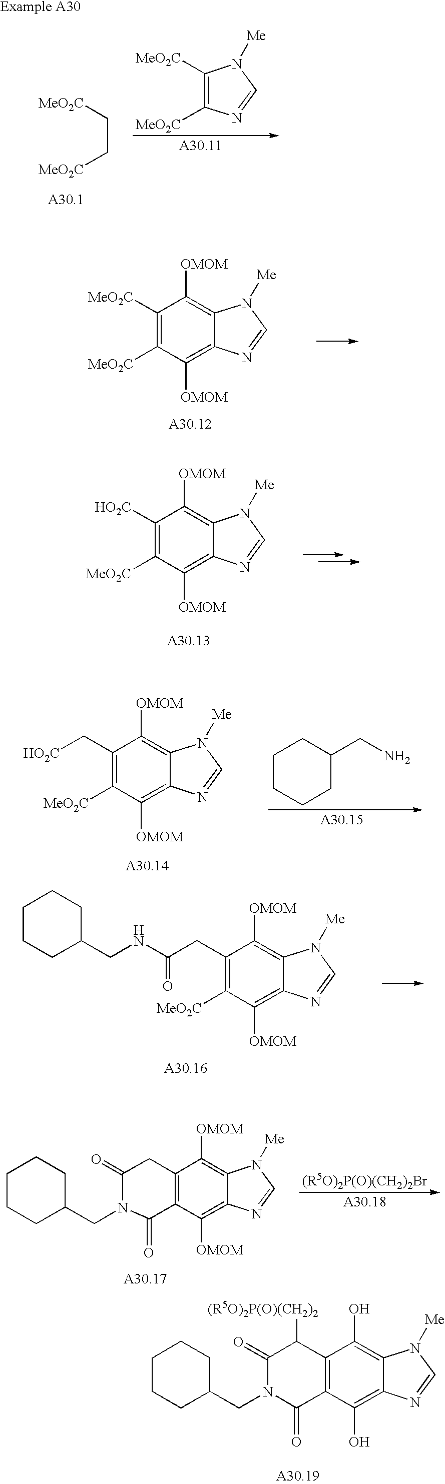
Preparation of the Intermediate Phosphonate Esters IVbb.

Schemes A31 and A32 illustrates the preparation of phosphonate esters of structure IVbb. Scheme A31 illustrates the preparation of phosphonate esters in which the phosphonate is attached by means of a variable carbon linkage linkage. In this procedure, the doubly protected phenol A29.3 is selectively deprotected to give the phenol A31.1. The product is converted into the triflate A31.2 and this material is reacted with a dialkyl hydroxy, mercapto or amino-substituted phosphonate A31.3, in which the group R is an acyclic or cyclic saturated or unsaturated alkylene, or aryl, aralkyl or heteroaryl moiety, in the presence of a base, as described in Scheme A8, to afford the displacement product A31.4, which upon deprotection gives the phenol A31.5.

For example, 2-naphthylmethylsuccinimide A31.6 is reacted with dimethyl pyrimidine 4,5-dicarboxylate A31.7 (Chem. Ber., 1975, 108, 3877) to afford after differential protection, as describe in Scheme 1 and Example 2 and triflate formation, trifluoro-methanesulfonic acid 7-naphthalen-2-ylmethyl-6,8-dioxo-9-triisopropylsilanyloxy-7,8-dihydro-6H-pyrrolo[3,4-g]quinazolin-5-yl ester A31.8. The compound is then reacted with a dialkyl 3-hydroxyphenyl phosphonate A31.9 (Aurora) and triethylamine in dichloromethane to give the phosphonate A31.10.

Using the above procedures, but employing, in place of the starting materials A31.6 and A31.7, different starting materials A29.3 and/or different phosphonates A31.3, the corresponding products A31.5 are obtained.

Scheme A32 depicts the preparation of phosphonate esters of structure Vbb in which the phosphonate is attached by means of an ether linkage. In this procedure, dimethyl succinate A32.1 is condensed under basic conditions, with a heterocyclic dicarboxylic ester A32.2 to afford the bicyclic product A32.3. Hydrolysis of the ester groups, followed by anhydride formation and selective protection of the phenolic hydroxyl groups, then gives the product A32.4. The anhydride is then reacted, as described on (06/03/0 page 31), with the substituted hydrazine A32.5, to yield the tricyclic product A32.6. Selective deprotection then affords the phenol A32.7, and this compound is then reacted with a dialkyl hydroxy-substituted phosphonate A32.8, in which the group R is an acyclic or cyclic saturated or unsaturated alkylene, or aryl, aralkyl or heteroaryl moiety, under the conditions of the Mitsonobu reaction, as described in Scheme A6, to form after deprotection the phenol A32.9.

For example, condensation between dimethyl succinate and dimethyl 1,3,4-triazine-5,6-dicarboxylate A32.10 (J. Org. Chem., 23, 1931, 1958) affords after selective silylation, following a procedure similar to Example 12, 6-(4-fluoro-benzyl)-9-hydroxy-10-triisopropylsilanyloxy-6,7-dihydro-1,2,4,6,7-pentaaza-anthracene-5,8-dione A32.11. The product is then reacted in tetrahydrofuran with a dialkyl hydroxyethyl phosphonate A32.12, (Epsilon) diethyl azodicarboxylate and triphenyl phosphine to yield after deprotection the phenolic phosphonate A32.13.

Using the above procedures, but employing, in place of the starting material A32.10 different starting materials A32.2 and/or different phosphonates A32.8, the corresponding products A32.9 are obtained. 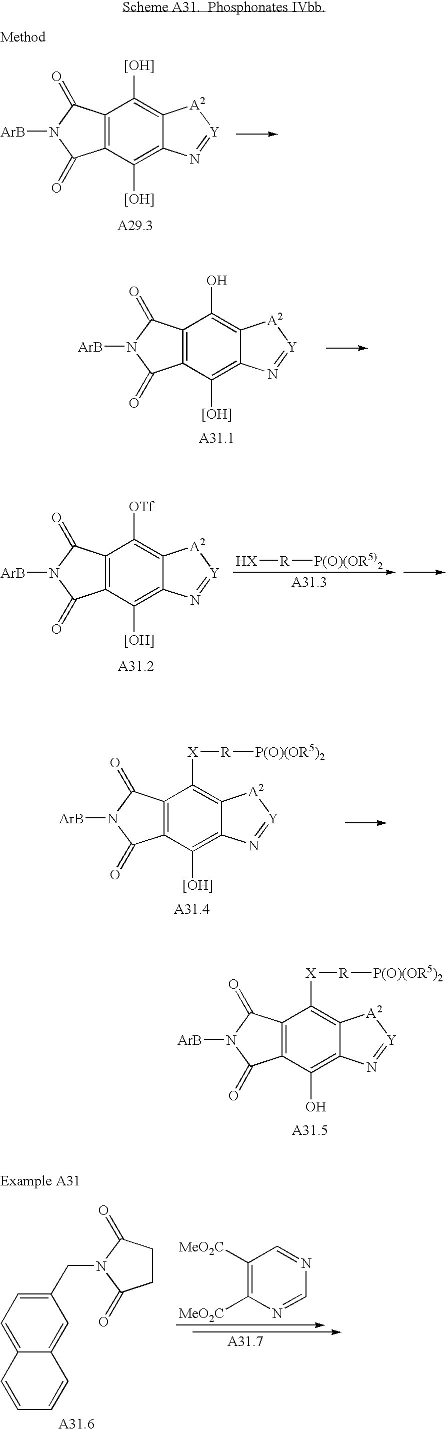 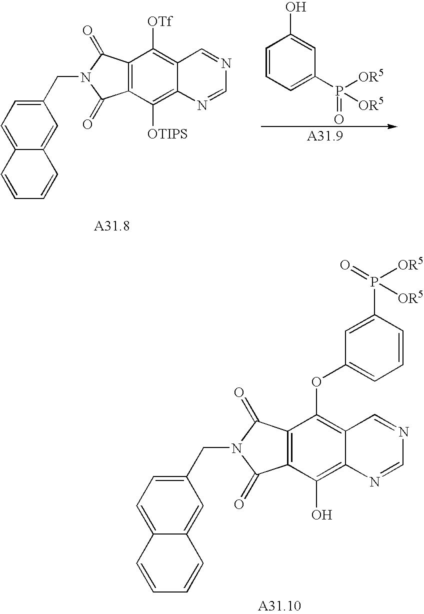 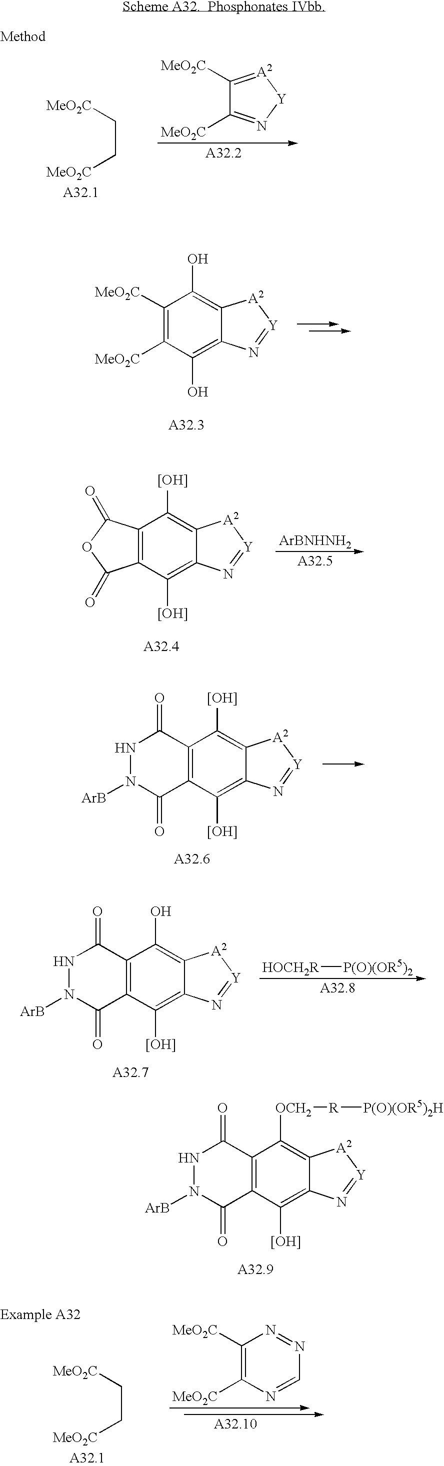 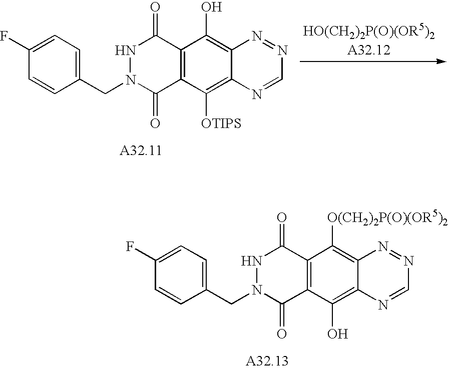
Preparation of the Intermediate Phosphonate Esters IVcc.

Scheme A33 illustrates the preparation of phosphonate esters of structure IVcc in which the phosphonate is attached by means of a carbon linkage. In this procedure, a substituted succinimide A33.1 is reacted with a heterocyclic diester A33.2 to afford after protection the bicyclic product A33.3. The amino group of the product is then alkylated by reaction with a dialkyl bromo-substituted phosphonate A33.4 to yield after deprotection the phenolic phosphonate A33.5.

For example, 1-(6-fluoro-1,2,3,4-tetrahydro-naphthalen-1-yl)-pyrrolidine-2,5-dione A33.6, prepared by the reaction of 2-amino-7-fluoro-1,2,3,4-tetrahydronaphthalene (U.S. Pat. No. 5,538,988) and succinic anhydride, is reacted with dimethyl 1,2,3-triazole-4,5-dicarboxylate A33.7 (Interchim) to afford after silylation of the phenolic hydroxyl groups 6-(6-fluoro-1,2,3,4-tetrahydro-naphthalen-1-yl)-4,8-bis-triisopropylsilanyloxy-1H-pyrrolo[3′,4′:4,5]benzo[1,2-d][1,2,3]triazole-5,7-dione A33.8. The product is then reacted, in dimethylformamide solution with one molar equivalent of sodium hydride and a dialkyl 4-bromobutyl phosphonate A33.9 (Syn., 1994, 9, 909) to afford after deprotection the phosphonate A33.10.

Using the above procedures, but employing, in place of the starting materials A33.6 and A33.7 different starting materials A33.1 and A33.2 and/or different phosphonates A33.4, the corresponding products A33.5 are obtained. 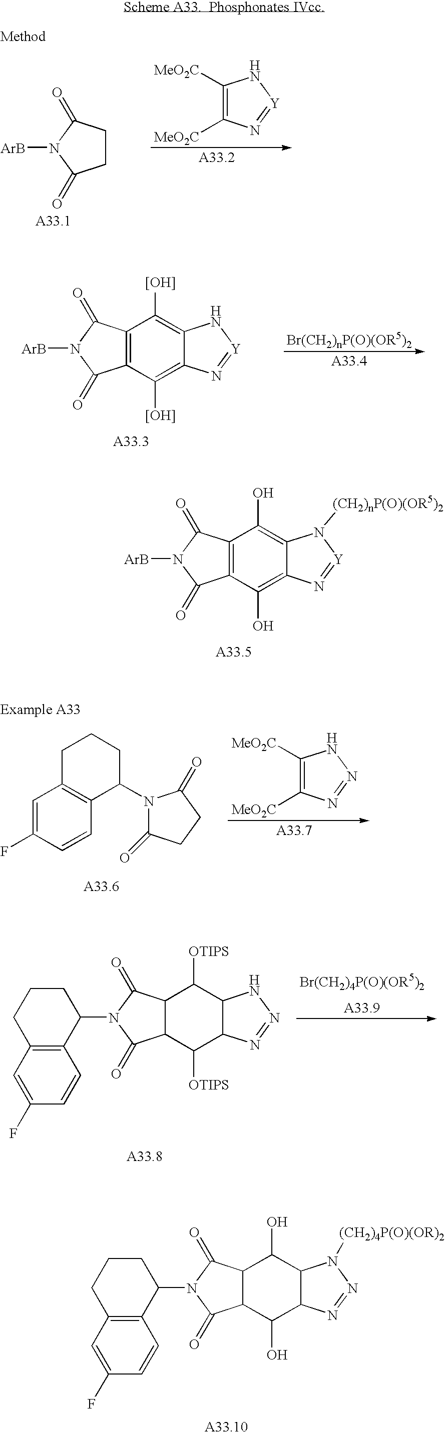
Synthesis of Formula II Aza-Quinolinol Phosphonate Compounds

Aza-quinolinol compounds have been prepared, including naphthyridine compounds (U.S. 2003/0119823 A1; WO 03/016315 A1; WO 03/016309 A1; WO 02/30930 A2; WO 02/055079 A2; WO 02/30931 A2; WO 02/30426 A1; WO 02/36734 A2). Quinoline derivatives have been reported (WO 03/031413 A1; U.S. 2002/0103220 A1; U.S. 2002/0055636 A1; U.S. Pat. No. 6,211,376; U.S. Pat. No. 6,114,349; U.S. Pat. No. 6,090,821; U.S. Pat. No. 5,883,255; U.S. Pat. No. 5,739,148; U.S. Pat. No. 5,639,881; U.S. Pat. No. 3,113,135).

Preparation of the Intermediate Phosphonate Esters 1-9.

The structures of the intermediate phosphonate esters 1-9 are shown in Chart 1, in which the substituent R¹ is H, alkyl, alkenyl, aryl or aralkyl, and the substituents R², R³, R⁴, X and X¹ are as previously defined. Subsequent chemical modifications to the compounds 1-9 as described below, permit the synthesis of the final compounds of this invention.

The intermediate compounds 1-9 incorporate a phosphonate group (R¹O)₂P(O) connected to the nucleus by means of a variable linking group, designated as “link” in the attached structures. Charts 2 and 3 illustrates examples of the linking groups present in the structures 1-9.

Schemes 1-31 illustrate the syntheses of the intermediate phosphonate compounds of this invention, 1-9, and of the intermediate compounds necessary for their synthesis.

The methods described for the introduction of phosphonate substituents are, with modifications made by one skilled in the art, transferable within the substrates 1-9. For example, reaction sequences which produce the phosphonates 1 are, with appropriate modifications, applicable to the preparation of the phosphonates 2-9. Methods described below for the attachment of phosphonate groups by means of reactive substituents such as OH, Br, NH₂, CH₃, CH₂Br, COOH, CHO etc are applicable to each of the scaffolds 1-9.

Scheme 32 illustrates methods for the interconversion of phosphonate diesters, monoesters and acids, and Scheme 33 illustrates methods for the preparation of carbamates. 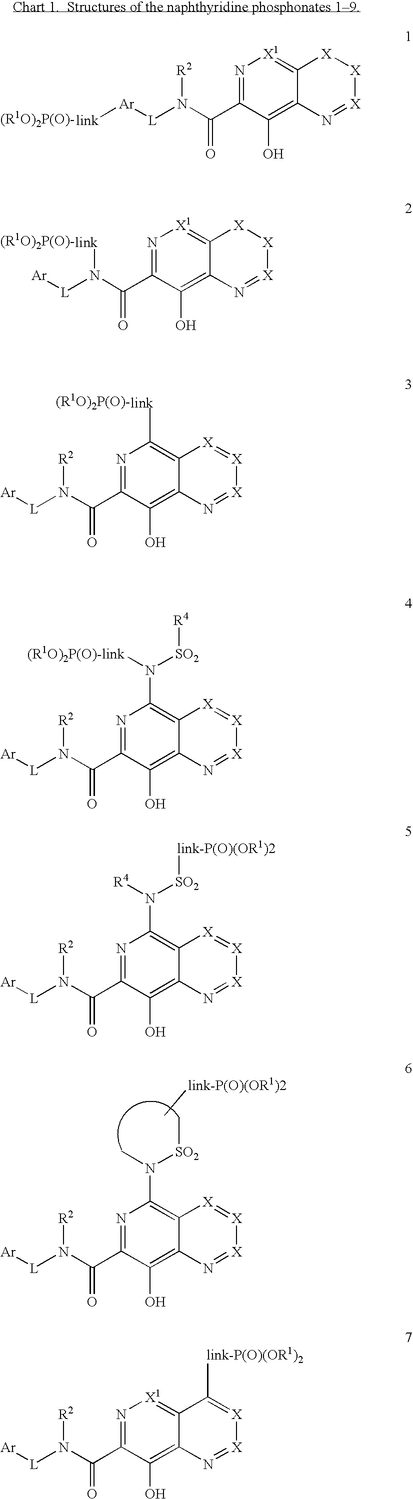 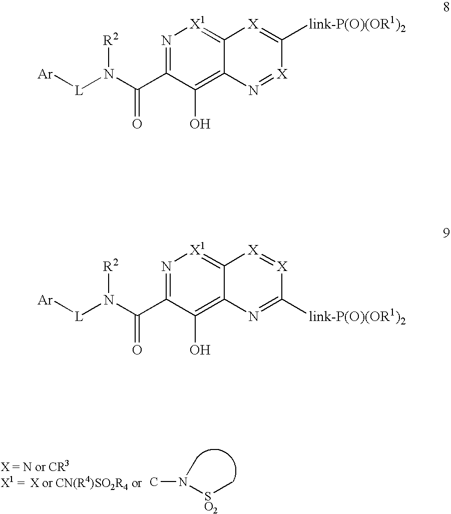 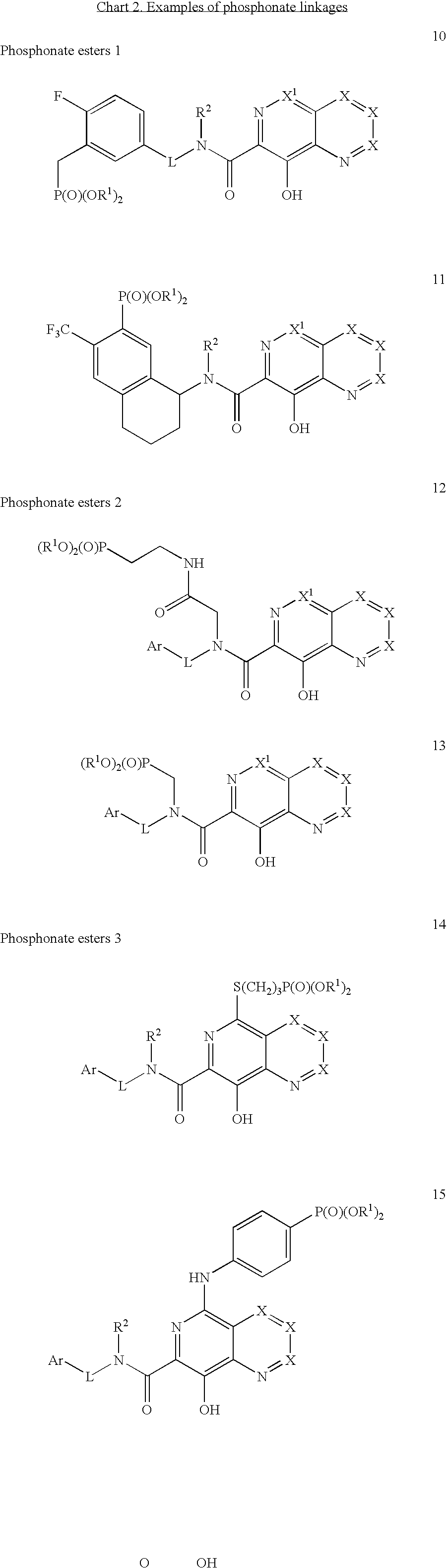 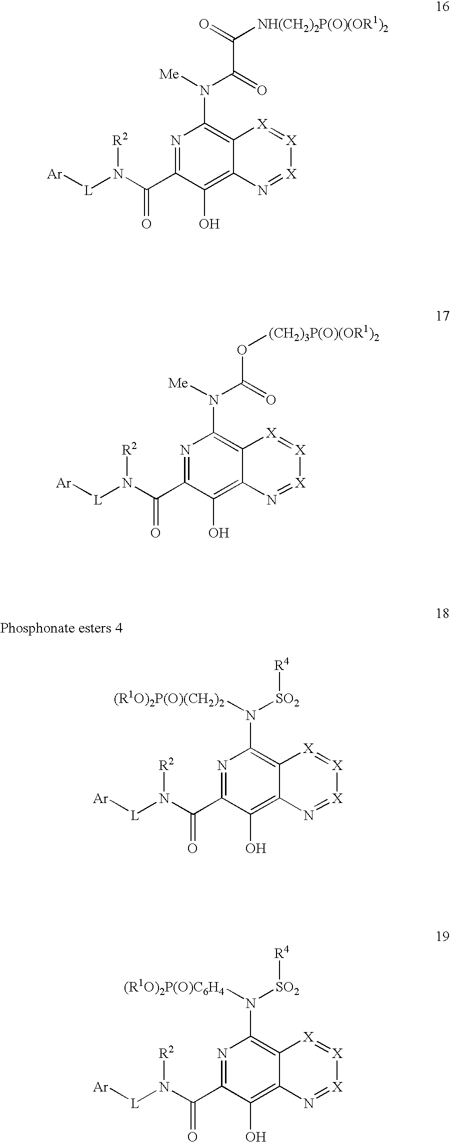 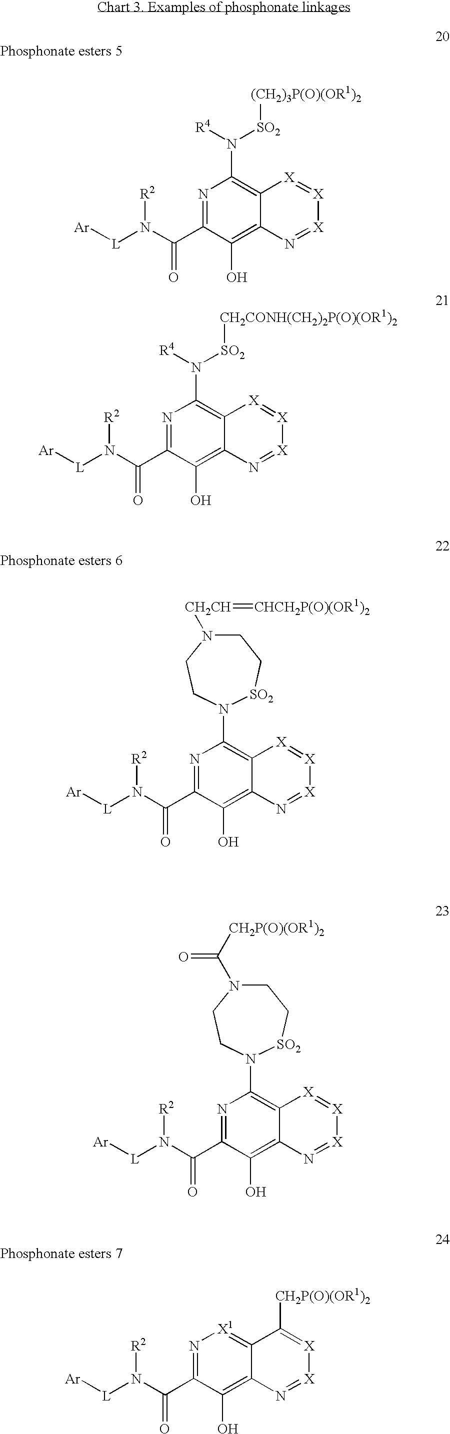 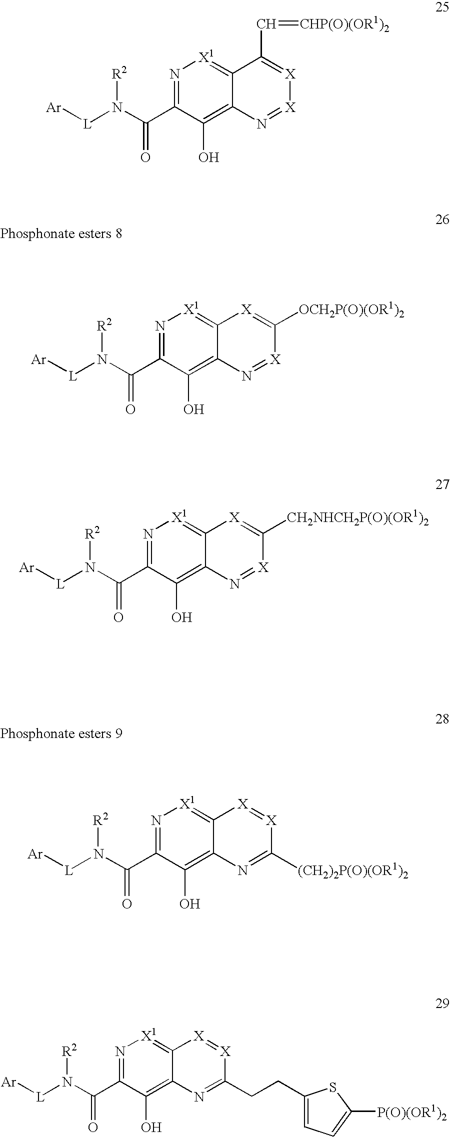
Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M. Wuts, Wiley, Second Edition 1990. Reactive substituents which may be protected are shown in the accompanying schemes as, for example, [OH], [SH], etc.

Preparation of the Intermediate Bicyclic Hydroxyesters and Hydroxyacids.

Scheme 1 illustrates the preparation of bicyclic hydroxyesters 1.2 from the corresponding anhydrides 1.1, in which at least one of the groups X is C—R³. The conversion is effected by means of one or more of the methods described in WO 0230930 A2, Schemes 2, 3, 3A and 5. The resultant ester is then converted into the carboxylic acid 1.3, for example by means of basic hydrolysis using sodium hydroxide, as described in WO 0230930 A2 Scheme 2.

As shown in Example 1, furo[3,4-c]pyridazine-5,7-dione 1.4 (WO 994492) is converted, as described above, into 8-hydroxy-pyrido[4,3-c]pyridazine-7-carboxylic acid methyl ester 1.5, and the ester is hydrolyzed with sodium hydroxide to give 8-hydroxy-pyrido[4,3-c]pyridazine-7-carboxylic acid 1.6.

In a similar manner, as shown in Examples 2 and 3, furo[3,4-b]pyrazine-5,7-dione 1.7 (Aldrich) and furo[3,4-e][1,2,4]triazine-5,7-dione 1.10 (J. Org. Chem., 1958, 23, 1931) are converted respectively into 8-hydroxy-pyrido[3,4-b]pyrazine-7-carboxylic acid methyl ester 1.8 and 8-hydroxy-pyrido[3,4-e][1,2,4]triazine-7-carboxylic acid methyl ester 1.11 and the corresponding carboxylic acids 1.9 and 1.12.

As shown in Example 4,3-methyl-furo[3,4-b]pyridine-5,7-dione 1.13 is converted, as described above, 8-hydroxy-3-methyl-[1,6]naphthyridine-7-carboxylic acid methyl ester 1.14 and the corresponding carboxylic acid 1.15.

Scheme 1A illustrates the preparation of bicyclic hydroxyesters 1A.3 in which a substituent Nu is introduced at the 5-position. In this procedure, the bicyclic hydroxyester 1A.1, prepared as described in Scheme 1, is halogenated to give the 5-halo product 1A.2 in which Ha is Cl, Br or I. The halogenation reaction is performed, for example, as described in WO 0230930 A2, p. 159, by reaction of the phenolic ester with N-bromosuccinimide in chloroform, to give the product 1A.2 in which Ha is Br. Alternatively, the hydroxyester 1A.1 is reacted with N-iodosuccinimide, as described in WO 0230930 A2 p. 166, to give the product 1A.2 in which Ha is iodo. The halogenated product is then reacted with a nucleophile Nu, to prepare the displacement product 1A.3. Examples of nucleophiles include hydroxy, mercapto or amino compounds, or cyclic or acyclic sulfonamides. The displacement reaction is performed in a polar organic solvent such as pyridine, dimethylformamide, DMPU, dimethylsulfoxide and the like, for example as described in WO 0230930 A2, Examples 57-78. Optionally, the phenolic hydroxyl group is protected prior to the displacement reaction, and deprotected afterwards.

For example, 8-hydroxy-[1,6]naphthyridine-7-carboxylic acid methyl ester 1A.4 (WO 0230930 A2, p. 171) is reacted with one molar equivalent of N-bromosuccinimide in dichloromethane, to yield 5-bromo-8-hydroxy-[1,6]naphthyridine-7-carboxylic acid methyl ester, 1A.5. The phenol is then reacted with p-toluenesulfonyl chloride and triethylamine in chloroform, for example as described in WO 0230931 A2 p. 72, to give 5-bromo-8-(toluene-4-sulfonyloxy)-[1,6]naphthyridine-7-carboxylic acid methyl ester 1A.6. The product is then reacted with [1,2]thiazinane 1,1-dioxide 1A.7 and cuprous oxide in pyridine at reflux, for example as described in WO 0230931 A2, p. 73, to produce 5-(1,1-dioxo-1,2]thiazinan-2-yl)-8-(toluene-4-sulfonyloxy)-[1,6]naphthyridine-7-carboxylic acid methyl ester 1A.8. Deprotection, for example by reaction with methanolic sodium methoxide in dimethylformamide, as described in WO 0230931 A2 p. 74, then affords the phenol 1A.9.

Using the above procedures, but employing different hydroxyesters 1A.1 in place of the hydroxyester 1A.4, and/or different nucleophiles, the corresponding products 1A.3 are obtained.

Alternative Methods for the Preparation of the Phosphonate Ester Amides 2.4.

As shown in Scheme 2, the hydroxyester 2.1, prepared as described above, is transformed, using the procedures described below, (Schemes 3-31) into the phosphonate ester 2.2. The ester, or the corresponding carboxylic acid, is then converted, using, for example, the procedures described in WO 0230930 A2, Schemes 1, 2, 3 and 5, into the phosphonate amide 2.4.

Alternatively, the ester 2.1, or the corresponding carboxylic acid, is transformed, as described above, into the amide 2.3, and the latter compound is then converted, using the procedures described below, (Schemes 3-31) into the phosphonate amide 2.4.

The selection of a suitable stage in the synthetic sequence for the introduction of the phosphonate group is made by one skilled in the art, depending on the reactivities and stabilities of the substrates in a given reaction sequence. 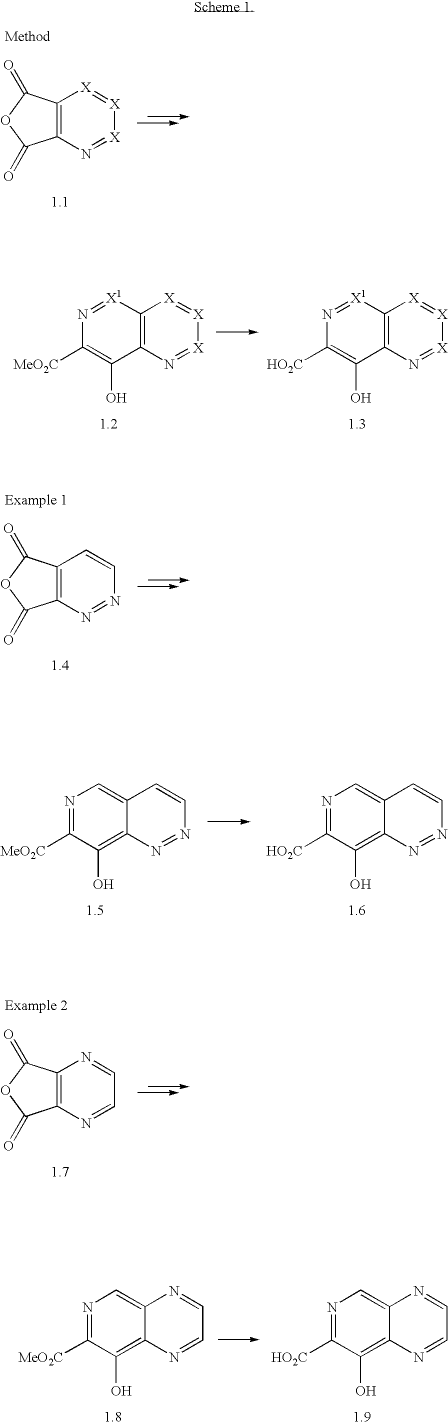 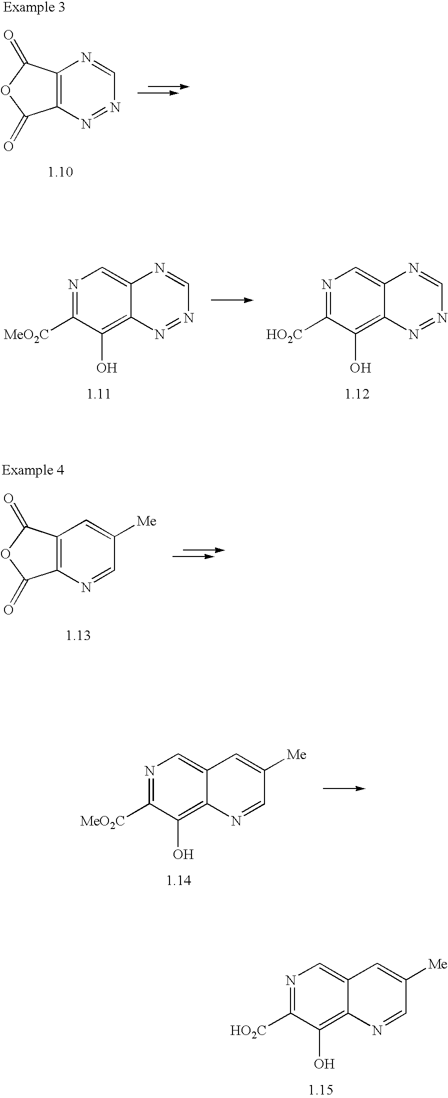 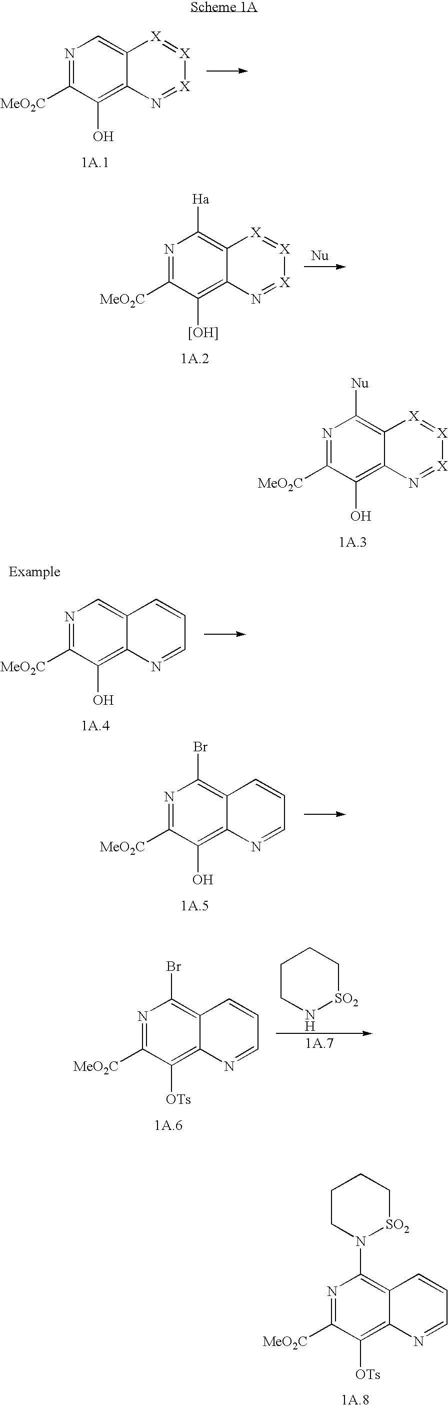 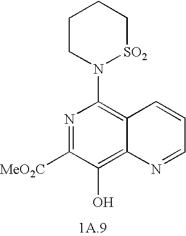 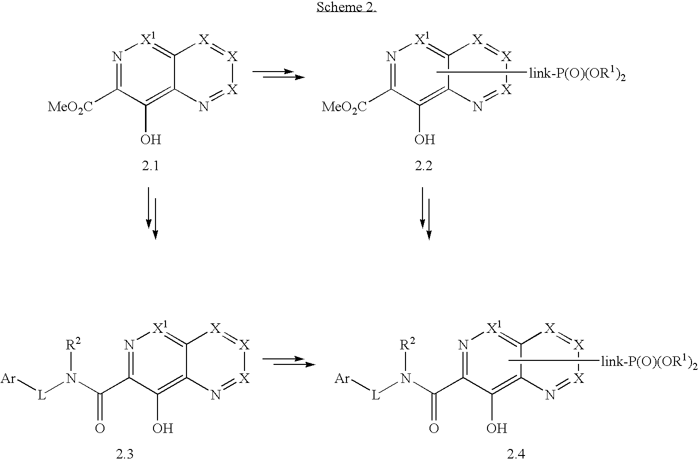
Preparation of the Intermediate Phosphonate Esters 1.

Schemes 3-7 illustrate methods for the preparation of the phosphonate esters 1.

Scheme 3 depicts the preparation of phosphonate esters 1 in which the phosphonate group is directly attached to the group Ar. In this procedure, a bromo-substituted amine 3.1, in which Ar is an aromatic or heteroaromatic group, is reacted, in the presence of a palladium catalyst, with a dialkyl phosphite 3.2 to yield the aryl phosphonate 3.3. The preparation of arylphosphonates by means of a coupling reaction between aryl bromides and dialkyl phosphites is described in J. Med. Chem., 35, 1371, 1992. This reaction is performed in an inert solvent such as toluene, in the presence of a base such as triethylamine and a palladium (0) catalyst such as tetrakis(triphenylphosphine)palladium(0). Optionally, the amine group is protected prior to the coupling reaction, and deprotected afterwards. The amine is then reacted with the ester 3.4 to afford the amide 3.5. The conversion of esters into amides is described in Comprehensive Organic Transformations, by R. C. Larock, VCH, 1989, p. 987. The reactants are combined in a solvent such as toluene or xylene, in the presence of a base such as sodium methoxide under azeotropic conditions, or of a dialkyl aluminum or trialkyl tin derivative of the amine. The use of trimethylaluminum in the conversion of esters to amides is described in J. Med. Chem. Chim. Ther., 34, 1999, 1995, and Syn. Comm., 25, 1401, 1995. The reaction is conducted in an inert solvent such as dichloromethane or toluene. The conversion of bicyclic esters such as 3.4, or the corresponding carboxylic acids, into amides is described in WO 0230930 A2, Schemes 1, 2, 3 and 6. Optionally, the phenolic hydroxyl group of the bicyclic ester 3.4 is protected, for example as a p-toluenesulfonyl derivative, as described in WO 0230930 A2, Example 1, prior to reaction with the amine component 3.3.

For example, 3-bromo-4-fluorobenzylamine 3.6 (Lancaster) is reacted in toluene solution at ca. 100°, with one molar equivalent of a dialkyl phosphite 3.7, triethylamine and 3 mol % of tetrakis(triphenylphosphine)palladium(0), to give the phosphonate product 3.8. The latter compound is then reacted, in toluene solution at reflux temperature with 5-(1,1-dioxo[1,2]thiazinan-2-yl)-8-hydroxy-[1,6]naphthyridine-7-carboxylic acid methyl ester 3.9, prepared by the methods described in WO 0230930 A2, and Schemes 1, 1A and 2, to yield the amide 3.10.

Using the above procedures, but employing, in place of the amine 3.6, different amines 3.1, and/or different bicyclic esters 3.4, the corresponding amides 3.5 are obtained.

Scheme 4 depicts the preparation of phosphonate esters 1 in which the phosphonate group is attached by means of a saturated or unsaturated alkylene chain. In this procedure, a bromo-substituted amine 4.1, in which Ar is an aryl or heteroaryl group, is subjected to a Heck coupling reaction, in the presence of a palladium catalyst, with a dialkyl alkenyl phosphonate 4.2, in which R⁵ is a direct bond, an alkyl, alkenyl, cycloalkyl or cycloalkenyl group, optionally incorporating a heteroatom O, S or N, or a functional group such as an amide, ester, oxime, sulfoxide or sulfone etc, or an optionally substituted aryl, heteroaryl or aralkyl group, to give the amine 4.3. The coupling of aryl halides with olefins by means of the Heck reaction is described, for example, in Advanced Organic Chemistry, by F. A. Carey and R. J. Sundberg, Plenum, 2001, p. 503ff and in Acc. Chem. Res., 12, 146, 1979. The aryl bromide and the olefin are coupled in a polar solvent such as dimethylformamide or dioxan, in the presence of a palladium(0) catalyst such as tetrakis(triphenylphosphine)palladium(0) or a palladium(II) catalyst such as palladium(II) acetate, and optionally in the presence of a base such as triethylamine or potassium carbonate. Optionally, the amine substituent is protected prior to the coupling reaction, and deprotected afterwards. The phosphonate amine 4.3 is then coupled, as described above, with the ester 4.4, or the corresponding carboxylic acid, to produce the amide 4.5. Optionally, the double bond is reduced to give the saturated analog 4.6. The reduction of olefinic bonds is described in Comprehensive Organic Transformations, by R. C. Larock, VCH, 1989, p. 6ff. The transformation is effected by means of catalytic hydrogenation, for example using a palladium on carbon catalyst and hydrogen or a hydrogen donor, or by the use of diimide or diborane.

For example, 3-bromo-4-methoxybenzylamine 4.7 (Lancaster) is reacted in dioxan solution with one molar equivalent of a dialkyl vinyl phosphonate 4.8 (Aldrich) and potassium carbonate, to yield the olefinic phosphonate 4.9. The product is then reacted, as described above, with 5-(1,1-dioxo-isothiazolidin-2-yl)-8-hydroxy-[1,6]naphthyridine-7-carboxylic acid methyl ester 4.10, prepared as described in Scheme 1A, and by methods described in WO 0230930 A2, to give the amide 4.11. The latter compound is reacted with diimide, prepared by basic hydrolysis of diethyl azodicarboxylate, as described in Angew. Chem. Int. Ed., 4, 271, 1965, to yield the saturated product 4.12.

Using the above procedures, but employing, in place of the amine 4.7, different amines 4.1, and/or different phosphonates 4.2, and/or different bicyclic esters 4.4, the corresponding amides 4.5 and 4.6 are obtained.

Scheme 5 depicts the preparation of phosphonate esters 1 in which the phosphonate group is attached by means of an amide linkage. In this procedure, the amine group of a carboxy-substituted amine 5.1 is protected to afford the derivative 5.2. The protection of amino groups is described in Protective Groups in Organic Synthesis, by T. W. Greene and P. G. M. Wuts, Wiley, Second Edition 1990, p. 309ff. Amino groups are protected, for example by alkylation, such as by mono or dibenzylation, or by acylation. The conversion of amines into mono or dibenzylamines, for example by treatment with benzyl bromide in a polar solvent such as acetonitrile or aqueous ethanol, in the presence of a base such as triethylamine or sodium carbonate, is described in Protective Groups in Organic Synthesis, by T. W. Greene and P. G. M. Wuts, Wiley, Second Edition 1990, p. 364. The N-protected carboxylic acid 5.2 is then coupled with an amino-substituted dialkyl phosphonate 5.3, in which the group R⁵ is as defined in Scheme 4, to yield the amide 5.4. The preparation of amides from carboxylic acids and derivatives is described, for example, in Organic Functional Group Preparations, by S. R. Sandler and W. Karo, Academic Press, 1968, p. 274, and in Comprehensive Organic Transformations, by R. C. Larock, VCH, 1989, p. 972ff. The carboxylic acid is reacted with the amine in the presence of an activating agent, such as, for example, dicyclohexylcarbodiimide or diisopropylcarbodiimide, optionally in the presence of, for example, hydroxybenztriazole, N-hydroxysuccinimide or N-hydroxypyridone, in a non-protic solvent such as, for example, pyridine, DMF or dichloromethane, to afford the amide.

Alternatively, the carboxylic acid is first converted into an activated derivative such as the acid chloride, anhydride, mixed anhydride, imidazolide and the like, and then reacted with the amine, in the presence of an organic base such as, for example, pyridine, to afford the amide.

The conversion of a carboxylic acid into the corresponding acid chloride is effected by treatment of the carboxylic acid with a reagent such as, for example, thionyl chloride or oxalyl chloride in an inert organic solvent such as dichloromethane, optionally in the presence of a catalytic amount of dimethylformamide.

The amino-protecting group is then removed from the product 5.4 to give the free amine 5.5. Deprotection of amines is described in Protective Groups in Organic Synthesis, by T. W. Greene and P. G. M. Wuts, Wiley, Second Edition 1990, p. 309ff. The amine is then coupled with the carboxylic acid 5.6, as described above, to produce the amide 5.7.

For example, 4-carboxycyclohexylmethylamine 5.8 (Aldrich) is converted into the phthalimido derivative 5.9. The conversion of amines into phthalimido derivatives is described in Protective Groups in Organic Synthesis, by T. W. Greene and P. G. M. Wuts, Wiley, Second Edition 1990, p. 358. The conversion is effected by reaction of the amine with an equimolar amount of 2-carbomethoxybenzoyl chloride, N-carboethoxyphthalimide, or preferably, phthalic anhydride. The reaction is performed in an inert solvent such as toluene, dichloromethane or acetonitrile, to prepare the phthalimido derivative 5.9. This material is then reacted with one molar equivalent of a dialkyl aminoethyl phosphonate 5.10, (J. Org. Chem., 2000, 65, 676) and dicyclohexylcarbodiimide in dimethylformamide, to give the amide 5.11. The phthalimido protecting group is then removed, for example by reaction with ethanolic hydrazine at ambient temperature, as described in J. Org. Chem., 43, 2320, 1978, to afford the amine 5.12. This compound is coupled in dimethylformamide solution with 5-(methanesulfonyl-methyl-amino)-8-hydroxy-[1,6]naphthyridine-7-carboxylic acid 5.13, prepared as described in Scheme 1A and WO 0230930 A2 Example 154, and 1-ethyl-3-(dimethylaminopropyl)carbodiimide, to afford the amide 5.14.

Using the above procedures, but employing, in place of the amine 5.8, different amines 5.1, and/or different phosphonates 5.3, and/or different carboxylic acids 5.6, the corresponding products 5.7 are obtained.

Scheme 6 depicts the preparation of phosphonates 1 in which the phosphonate is attached by means of an ether linkage. In this procedure, the amino group of a hydroxy-substituted amine 6.1 is protected, as described above, to give the derivative 6.2. The alcohol is then reacted, with base catalysis, with a dialkyl bromomethyl phosphonate 6.3, in which the group R⁵ is as defined in Scheme 4. The reaction is conducted in a polar aprotic solvent such as tetrahydrofuran, dimethylformamide or dimethylsulfoxide, in the presence of a base such as potassium carbonate, for cases in which Ar is an aromatic group, or a strong base such as sodium hydride, for cases in which Ar is an aliphatic group. The amino group of the resulting ether 6.4 is then deprotected, as previously described, to give the amine 6.5. The amine is then reacted with the ester 6.6, as described in Scheme 3, to give the amide 6.7.

For example, N-methyl 3-hydroxyphenethylamine 6.8 is reacted with one molar equivalent of acetyl chloride in dichloromethane containing pyridine, to give the N-acetyl product 6.9. The product is then reacted at ca. 60° in dimethylformamide solution with one molar equivalent of a dialkyl 3-bromopropenyl phosphonate 6.10 (Aurora) and cesium carbonate, to produce the ether 6.11. The N-acetyl group is then removed, for example by treatment with hog kidney acylase, as described in Tet., 44, 5375, 1988, to give the amine 6.12. The product is then reacted in toluene solution at reflux, as described above, with 5-(1,1-dioxo-[1,2]thiazepan-2-yl)-8-hydroxy-[1,6]naphthyridine-7-carboxylic acid methyl ester 6.13, prepared as described in Scheme 1A and in WO 0230931 Example 6, to yield the amide 6.14.

Using the above procedures, but employing, in place of the amine 6.8, different amines 6.1, and/or different phosphonates 6.3, and/or different bicyclic esters 6.6, the corresponding products 6.7 are obtained.

Scheme 7 depicts the preparation of phosphonates 1 in which the phosphonate is attached by means of an ether or thioether linkage. In this procedure, a N-protected hydroxyamine 6.2, in which Ar is an aromatic moiety, is subjected to a Mitsunobu reaction with a hydroxy or mercapto-substituted dialkyl phosphonate 7.1, in which R⁵ is as defined in Scheme 4, to prepare the ether or thioether product 7.2. The preparation of aromatic ethers and thioethers by means of the Mitsunobu reaction is described, for example, in Comprehensive Organic Transformations, by R. C. Larock, VCH, 1989, p. 448, and in Advanced Organic Chemistry, Part B, by F. A. Carey and R. J. Sundberg, Plenum, 2001, p. 153-4 and in Org. React., 1992, 42, 335. The phenol and the alcohol or thiol component are reacted together in an aprotic solvent such as, for example, tetrahydrofuran or dioxan, in the presence of a dialkyl azodicarboxylate and a triarylphosphine, to afford the ether or thioether products. The N-protecting group is then removed and the resultant amine is converted, as described in Scheme 6, into the amide 7.3.

For example, N-acetyl 3,5-dichloro-4-hydroxybenzylamine 7.4 is reacted tetrahydrofuran solution with one molar equivalent of a dialkyl mercaptoethyl phosphonate 7.5, (Zh. Obschei. Khim., 1973, 43, 2364) diethyl azodicarboxylate and tri-o-tolylphosphine, to afford the thioether product 7.6. The N-acetyl group is removed, as described in Scheme 6, and the amine 7.7 is then reacted with 5-(1,1-dioxo-[1,2,5]thiadiazepan-2-yl)-8-hydroxy-[1,6]naphthyridine-7-carboxylic acid methyl ester 7.8, (see, for example, WO 0230931 Example 3) to afford the amide 7.9.

Using the above procedures, but employing, in place of the amine 7.4, different amines 6.2, and/or different phosphonates 7.2, the corresponding products 7.3 are obtained. 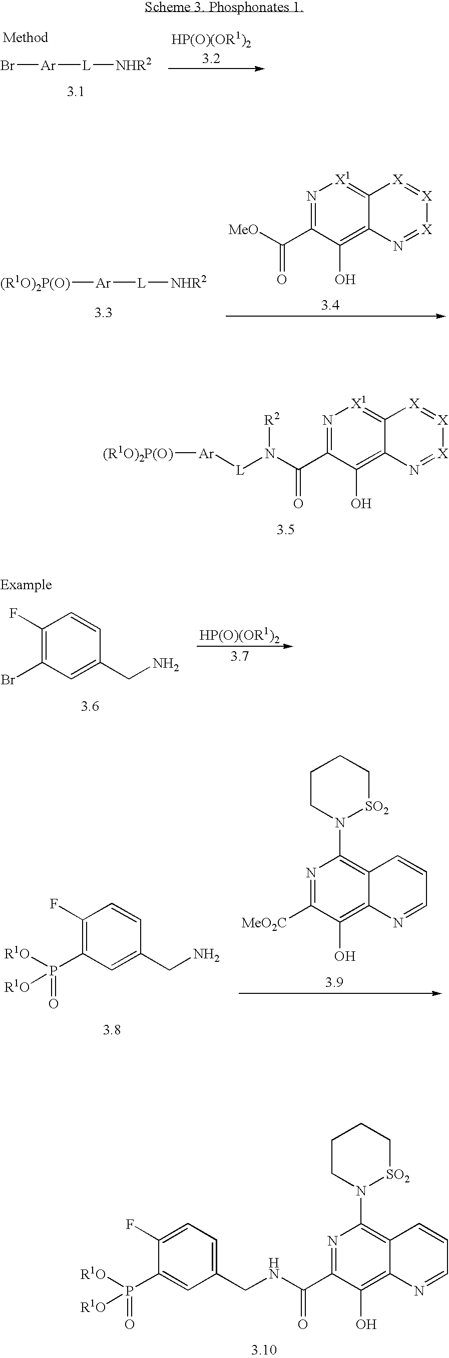 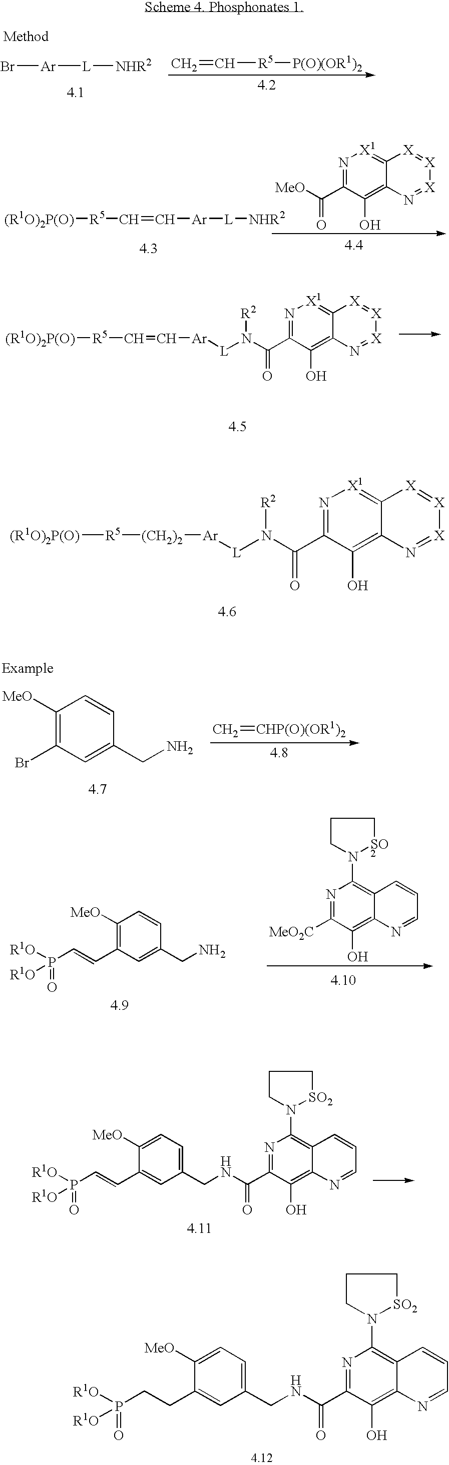 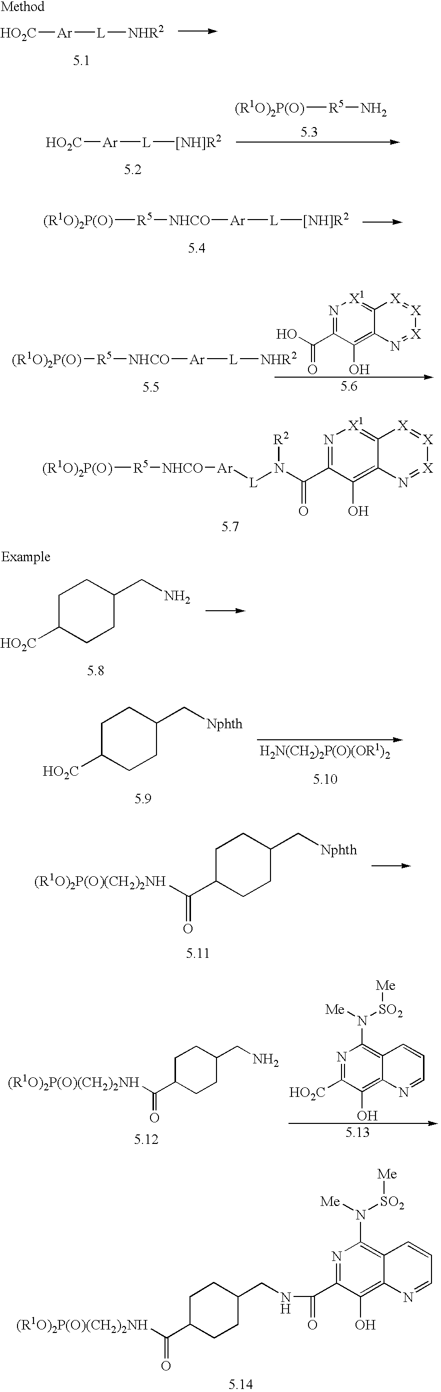 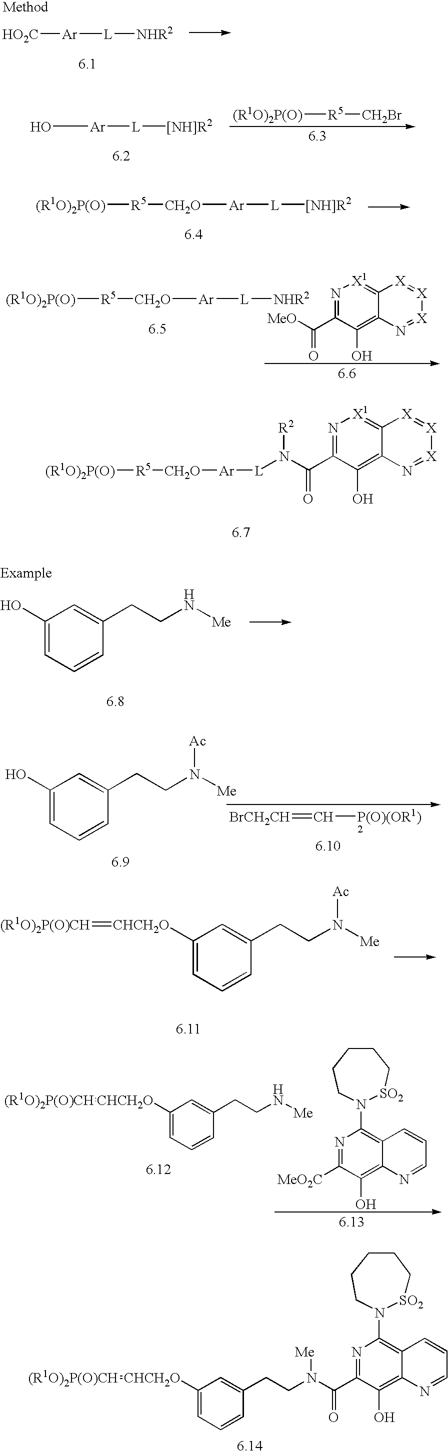
Preparation of the Intermediate Phosphonate Esters 2.

Schemes 8-10 illustrate methods for the preparation of the phosphonate esters 2.

Scheme 8 depicts the preparation of phosphonates 2 in which the phosphonate is attached by means of an alkylene chain incorporating an amide linkage. In this procedure, an amine 8.1 is reacted with a bromoalkyl ester 8.2, in which R⁵ is as defined in Scheme 4, to yield the alkylated amine 8.3. The preparation of substituted amines by the reaction of amines with alkyl halides is described, for example, in Comprehensive Organic Transformations, by R. C. Larock, VCH, 1989, p. 397. Equimolar amounts of the reactants are combined in a polar solvent such as an alkanol or dimethylformamide and the like, in the presence of a base such as cesium carbonate, diazabicyclononene or dimethylaminopyridine, to yield the substituted amine. The ester group is then hydrolyzed to give the carboxylic acid 8.4, and this compound is then coupled, as described in Scheme 5, with a dialkyl aminoalkyl phosphonate 8.5, to produce the aminoamide 8.6. Optionally, the amino group of the amine 8.4 is protected prior to the coupling reaction, and deprotected afterwards. The product is then reacted with the bicyclic hydroxyester 8.7 to afford the amide 8.8.

For example, 4-trifluoromethylbenzylamine 8.9 is reacted in dimethylformamide with one molar equivalent of methyl bromoacetate 8.10 and potassium carbonate to give the ester 8.11. Hydrolysis, employing one molar equivalent of lithium hydroxide in aqueous dimethoxyethane, affords the carboxylic acid 8.12, and this compound is coupled in tetrahydrofuran solution with a dialkyl aminomethyl phosphonate 8.13 (Aurora), in the presence of dicyclohexylcarbodiimide, to give the aminoamide 8.14. The product is then reacted with 5-(1,1-dioxo-isothiazolidin-2-yl)-8-hydroxy-[1,6]naphthyridine-7-carboxylic acid methyl ester 8.15, prepared by the methods described above, to yield the amide 8.16.

Using the above procedures, but employing, in place of the amine 8.9, different amines 8.1, and/or different bromoesters 8.2, and/or different phosphonates 8.5, and/or different hydroxyesters 8.7, the corresponding products 8.8 are obtained.

Scheme 9 depicts the preparation of phosphonates 2 in which the phosphonate is attached by means of a variable carbon chain. In this procedure, a primary amine 9.1 is subjected to a reductive amination reaction with a dialkyl formyl-substituted phosphonate 9.2, in which R⁵ is as defined in Scheme 4, to afford the alkylated amine 9.3. The preparation of amines by means of reductive amination procedures is described, for example, in Comprehensive Organic Transformations, by R. C. Larock, VCH, p. 421, and in Advanced Organic Chemistry, Part B, by F. A. Carey and R. J. Sundberg, Plenum, 2001, p. 269. In this procedure, the amine component and the aldehyde or ketone component are reacted together in a polar solvent in the presence of a reducing agent such as, for example, borane, sodium cyanoborohydride, sodium triacetoxyborohydride or diisobutylaluminum hydride, optionally in the presence of a Lewis acid, such as titanium tetraisopropoxide, as described in J. Org. Chem., 55, 2552, 1990. The product 9.3 is then reacted, as described previously, with the bicyclic ester 9.4 to give the amide 9.5.

For example, 3,4-dichlorobenzylamine is reacted in methanol solution with one molar equivalent of a dialkyl 3-formylphenyl phosphonate 9.7, (Epsilon) and sodium cyanoborohydride, to yield the alkylated product 9.8. This compound is then reacted with 5-(methanesulfonyl-methyl-amino)-8-hydroxy-[1,6]naphthyridine-7-carboxylic acid methyl ester 9.9, prepared using the methods described above, from the corresponding bromo compound and N-methyl methanesulfonamide, to give the amide 9.10.

Using the above procedures, but employing, in place of the amine 9.6, different amines 9.1, and/or different phosphonates 9.2, and/or different bicyclic esters 9.4, the corresponding products 9.5 are obtained.

Scheme 10 depicts an alternative method for the preparation of phosphonates 2 in which the phosphonate is attached by means of a variable carbon chain. In this procedure, the phenolic group of a bicyclic amide 10.1, prepared as described above, and in WO 02 30930 A2, is protected to give the product 10.2. The protection of phenolic hydroxyl groups is described in Protective Groups in Organic Synthesis, by T. W. Greene and P. G. M. Wuts, Wiley, Second Edition 1990, p. 10ff. For example, hydroxyl substituents are protected as trialkylsilyloxy ethers. Trialkylsilyl groups are introduced by the reaction of the phenol with a chlorotrialkylsilane and a base such as imidazole, for example as described in Protective Groups in Organic Synthesis, by T. W. Greene and P. G. M. Wuts, Wiley, Second Edition 1990, p. 10, p. 68-86. Alternatively, phenolic hydroxyl groups are protected as benzyl or substituted benzyl ethers, or as acetal ethers such as methoxymethyl or tetrahydropyranyl. The O-protected amide 10.2 is then reacted with the phosphonate-substituted trifluoromethanesulfonate 10.3, in which R⁵ is as defined in Scheme 4, to produce the alkylated amide 10.4. The alkylation reaction is conducted between equimolar amounts of the reactants in an aprotic organic solvent such as dimethylformamide or dioxan, in the presence of a strong base such as lithium hexamethyl disilylazide or sodium hydride, at from ambient temperature to about 90°. The hydroxyl group is then deprotected to give the phenol 10.5. Deprotection of phenolic hydroxyl groups is described in Protective Groups in Organic Synthesis, by T. W. Greene and P. G. M. Wuts, Wiley, Second Edition 1990, p. 10ff. For example, silyl protecting groups are removed by reaction with tetrabutylammonium fluoride, benzyl groups are removed by catalytic hydrogenation and acetal ethers are removed by treatment with acids.

For example, furo[3,4-b]pyrazine-5,7-dione 10.6, (J. Org. Chem., 1964, 29, 2128) is converted, as described above, (Schemes 1, 1A and 2) and in WO 0230930 A2, into 5-(1,1-dioxo-1,2]thiazinan-2-yl)-8-hydroxy-pyrido[3,4-b]pyrazine-7-carboxylic acid (naphthalen-2-ylmethyl)-amide 10.7. The product is then reacted with one molar equivalent of tert-butyl chlorodimethylsilane and imidazole in dichloromethane, to give the silyl ether 10.8. This compound is then reacted at ambient temperature in dioxan solution with one molar equivalent of sodium hydride, followed by the addition of a dialkyl trifluoromethanesulfonyloxymethyl phosphonate 10.9 (Tet. Lett., 1986, 27, 1477), to afford the alkylated product 10.10. Deprotection, by reaction with tetrabutylammonium fluoride in tetrahydrofuran, then yields the product 10.11.

Using the above procedures, but employing, in place of the amide 10.7, different amides 10.1, and/or different phosphonates 10.3, the corresponding products 10.5 are obtained. 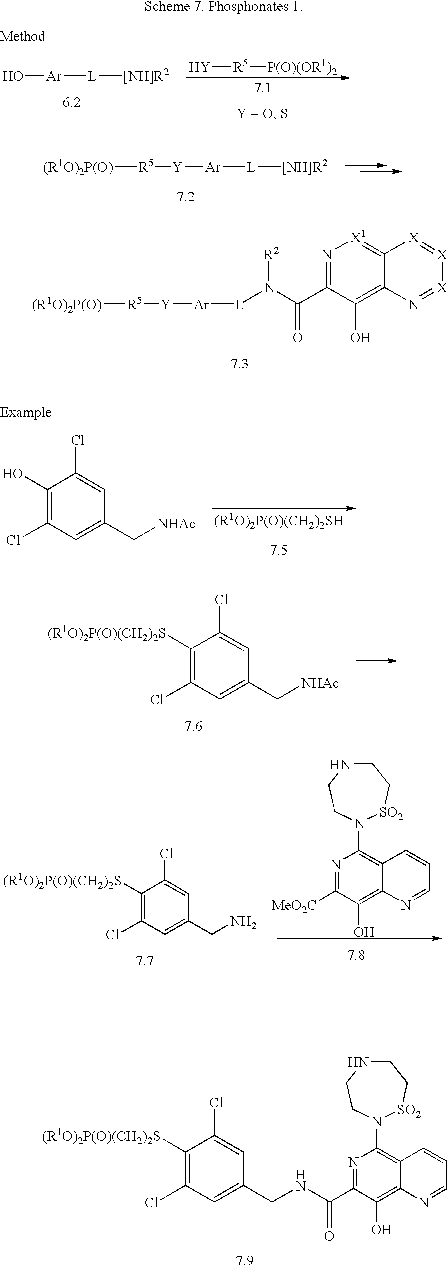 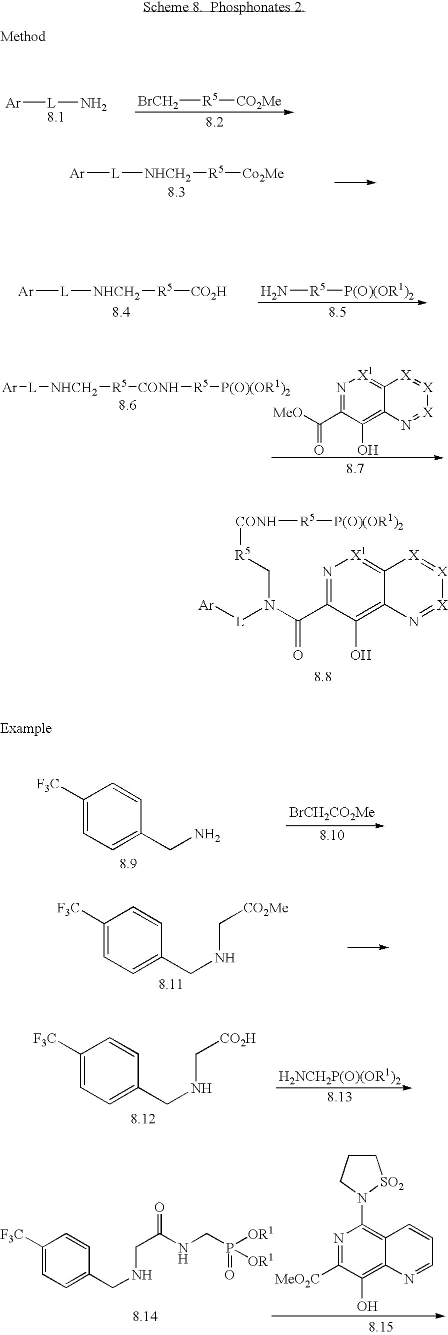 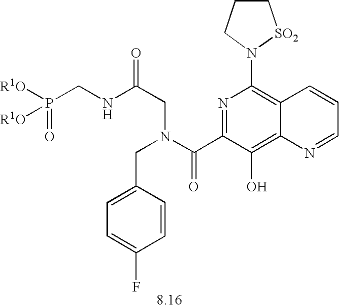 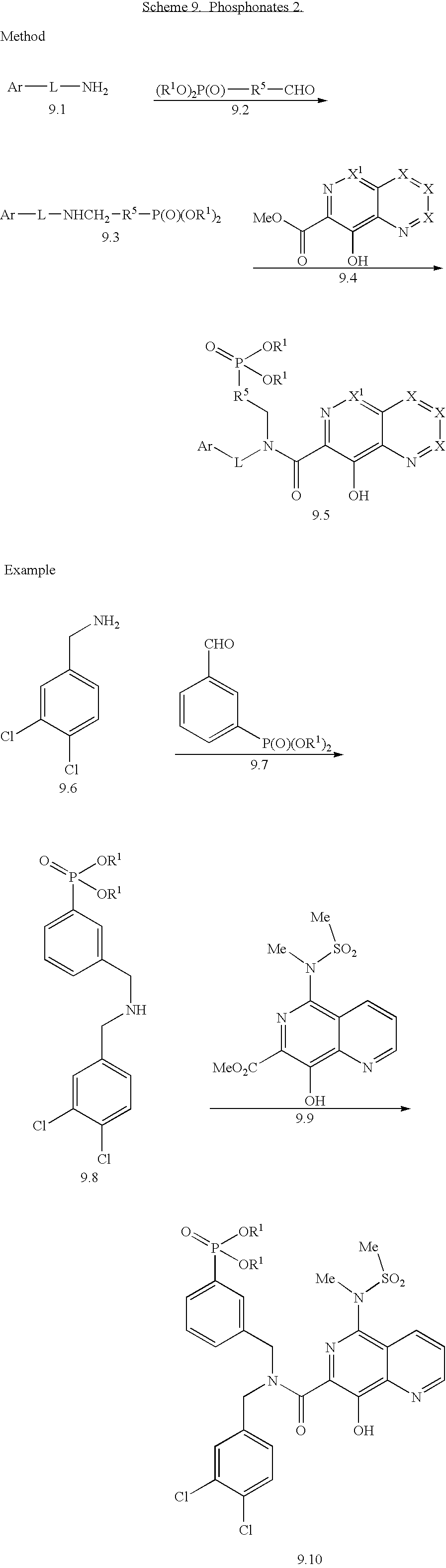 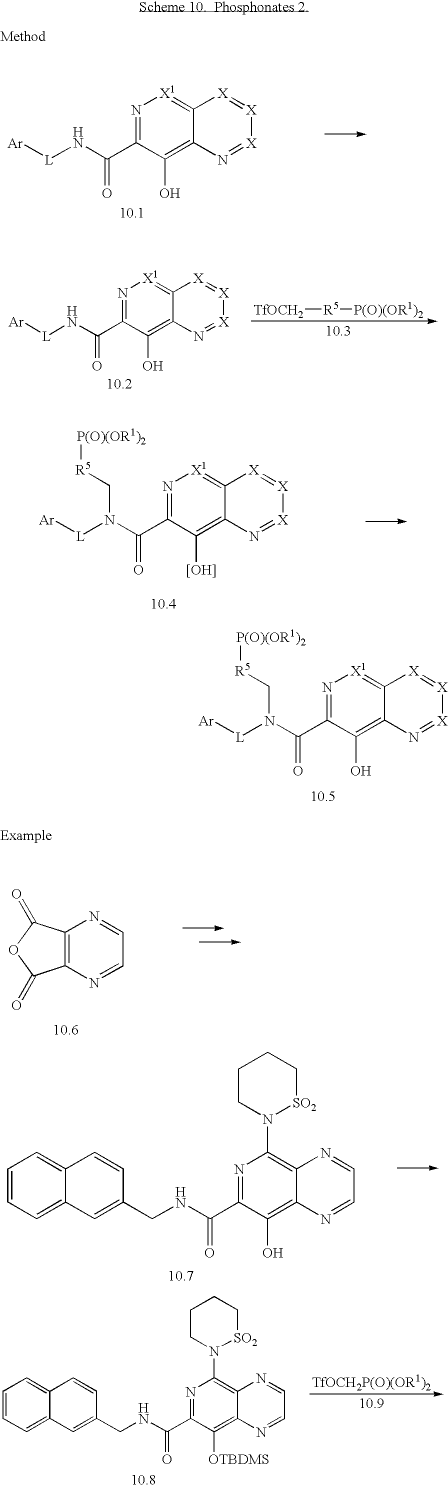 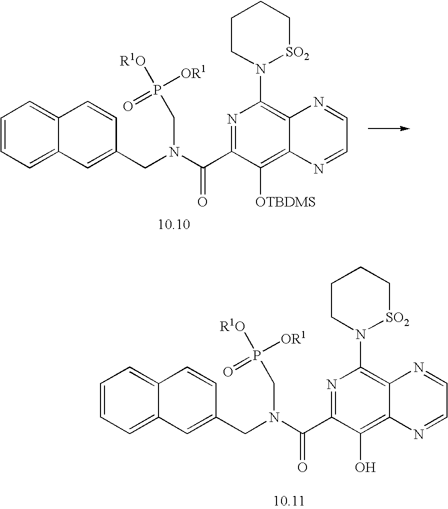
Preparation of the Intermediate Phosphonate Esters 3.

Schemes 11-15 illustrate methods for the preparation of the phosphonate esters 3.

Scheme 11 depicts the preparation of phosphonates 3 in which the phosphonate is attached by means of a heteroatom O, S or N, and a variable carbon chain. In this procedure, a bicyclic amide 11.1, prepared as previously described, is reacted in an aprotic solvent such as dichloromethane, hexachloroethane or ethyl acetate with a free radical brominating agent such as N-bromosuccinimide or N-bromoacetamide, to yield the 5-bromo product 11.2. This compound is then reacted with a dialkyl hydroxy, mercapto or amino-substituted phosphonate 11.3, in which R⁵ is as defined as in Scheme 4, to give the ether, thioether or amine product 11.4. The displacement reaction is conducted in a polar aprotic organic solvent such as dimethylformamide or DMPU, at from 100° to about 150°, in the presence of a base such as triethylamine or cesium carbonate, for example as described in WO 0230930 A2, Examples 57-69.

As shown in Example 1, furo[3,4-d]pyrimidine-5,7-dione 11.5 (J. Het. Chem., 1993, 30, 1597) is converted, as described above, into 8-hydroxy-pyrido[4,3-d]pyrimidine-7-carboxylic acid cyclohexylmethyl-amide 11.6. The product is reacted with one molar equivalent of N-bromosuccinimide in dichloromethane to yield the 5-bromo product 11.7. This material is then reacted with a dialkyl mercaptoethyl phosphonate 11.8 (Zh. Obschei. Khim., 1973, 43, 2364) and triethylamine at ca 100° in a pressure vessel, to produce the thioether 11.9.

As shown in Example 2, the anhydride 11.10 is converted, as described previously, into 8-hydroxy-[1,6]naphthyridine-7-carboxylic acid 3,5-dichloro-benzylamide 11.11. Bromination with N-bromosuccinimide in ethyl acetate at reflux temperature then yields the bromo compound 11.12 which is reacted with a dialkyl 3-aminophenyl phosphonate 11.13 (J. Med. Chem., 1984, 27, 654) in dimethylformamide at ca. 130′, using the procedure described in WO 0230930 A2 Example 63, to give the phosphonate 11.14. The product is then reacted with N,N-dimethyloxamide 11.15, (Japanese Patent 540467 18) and dicyclohexylcarbodiimide in dimethylformamide, to yield the amide product 11.16.

Using the above procedures, but employing, in place of the amides 11.6 or 11.11, different amides 11.1, and/or different phosphonates 11.3, the corresponding products 11.4 are obtained.

Scheme 12 depicts the preparation of phosphonates 3 in which the phosphonate is attached by means of a carbamate linkage. In this procedure, a protected bromophenol 12.1 is reacted, as described in Scheme 11, with an amine 12.2 to give the displacement product 12.3. This compound is then reacted with phosgene, triphosgene, carbonyl diimidazole or a functional equivalent thereof, and a dialkyl hydroxyalkyl phosphonate 12.4, in which R⁵ is as defined in Scheme 4, to yield, after deprotection of the phenol, the carbamate 12.5. Various methods for the preparation of carbamates are described in Scheme 33.

For example, the hydroxyester 12.6 is converted, as described previously, into the amide 12.7. This material is then reacted, in dimethylformamide solution at 100°, with ethylamine and cesium carbonate in dimethylformamide, to afford 8-(tert-butyl-dimethyl-silanyloxy)-5-ethylamino-[1,6]naphthyridine-7-carboxylic acid [2-(4-fluoro-phenyl)-cyclopropyl]-amide 12.9. The amine is treated with equimolar amounts of a dialkyl hydroxypropyl phosphonate 12.10 (Zh. Obschei. Khim., 1974, 44, 1834) and carbonyldiimidazole in dichloromethane, to prepare, after desilylation, the carbamate 12.11.

Using the above procedures, but employing, in place of the amide 12.7, different amides 12.3, and/or different phosphonates 12.4, the corresponding products 12.5 are obtained.

Scheme 13 depicts the preparation of phosphonates 3 in which the phosphonate is attached by means of an arylvinyl or arylethyl linkage. In this procedure, a bromophenol 13.1 is protected to give the product 13.2. This compound is then coupled with tributylvinyltin to yield the 5-vinyl product 13.3. The coupling reaction is effected in dimethylformamide solution at ca. 80° in the presence of a palladium(0) catalyst, such as tris(dibenzylideneacetone)palladium(0), a triarylphosphine such as tri(2-furyl)phosphine and copper(I) iodide, for example as described in WO 0230930A2, Example 176. The vinyl-substituted product is subjected to a palladium-catalyzed Heck coupling reaction, as described in Scheme 4, with a dibromoaromatic or heteroaromatic compound 13.4, to give the bromoaryl product 13.5. The latter compound is then coupled, as described in Scheme 3, with a dialkyl phosphite 13.6, in the presence of a palladium catalyst, to give the aryl phosphonate 13.7. Deprotection then affords the phenol 13.8. Optionally, the double bond is reduced, for example as described in Scheme 4, to give the saturated analog 13.9.

For example, furo[3,4-c]pyridazine-5,7-dione 13.10, (WO9944992) is converted, using the methods described above, into the silyl-protected bromophenol 13.11. The product is coupled, as described above, with tri(n-butyl)vinyltin to produce the 5-vinyl compound 13.12. This material is then coupled, in dimethylformamide solution at 80° with one molar equivalent of 2,5-dibromothiophene, in the presence of tetrakis(triphenylphosphine)palladium(0) and triethylamine, to afford 5-[2-(5-bromo-thiophen-2-yl)-vinyl]-8-(tert-butyl-dimethyl-silanyloxy)-pyrido[4,3-c]pyridazine-7-carboxylic acid 3,5-dichloro-benzylamide 13.14. The product is coupled, in the presence of a palladium(0) catalyst and triethylamine, with a dialkyl phosphite 13.15, to afford the phosphonate 13.16. Deprotection, for example by reaction with tetrabutylammonium fluoride in tetrahydrofuran, then yields the phenol 13.17, and hydrogenation of the latter compound in methanol, using 5% palladium on carbon as catalyst, produces the saturated analog 13.18.

Using the above procedures, but employing, in place of the amide 13.11, different amides 13.1, and/or different dibromides 13.4, the corresponding products 13.8 and 13.9 are obtained.

Scheme 14 depicts the preparation of phosphonates 3 in which the phosphonate is attached by means of an acetylenic bond. In this procedure, a phenol 14.1 is reacted, as described in WO 0230930 A2 p. 166 and Example 112, with N-iodosuccinimide in dichloromethane-dimethylformamide, to give the 5-iodo product; protection of the phenolic hydroxyl group then affords the compound 14.2. This material is coupled, as described in WO 0230930 A2 Example 79, in dimethylformamide solution, in the presence of dichlorobis(triphenylphosphine) palladium (II), copper iodide and triethylamine, with a dialkyl ethynyl phosphonate 14.3, in which R⁵ is as defined in Scheme 4, to give, after deprotection of the phenol, the acetylenic phosphonate 14.4.

For example, furo[3,4-e][1,2,4]triazine-5,7-dione 14.5, (J. Org. Chem., 1958, 23, 1931) is converted, as described previously, into the hydroxyester 14.6. This material is then converted into 5-iodo-8-(tert-butyl-dimethyl-silanyloxy)-pyrido[3,4-e][1,2,4]triazine-7-carboxylic acid (cyclopent-3-enylmethyl)-amide 14.7, as described above. The product is coupled, as described above, with a dialkyl propynyl phosphonate 14.8, (Syn., 1999, 2027) to yield, after deprotection, the acetylenic phosphonate 14.9.

Using the above procedures, but employing, in place of the iodoamide 14.7, different iodoamides 14.2, and/or different acetylenic phosphonates 14.3, the corresponding products 14.4 are obtained.

Scheme 15 depicts the preparation of phosphonates 3 in which the phosphonate is directly attached to the bicyclic nucleus. In this procedure, a protected bicyclic bromophenol 15.1 is coupled, in the presence of a palladium catalyst, as described in Scheme 3, with a dialkyl phosphite 15.2, to give after deprotection the aryl phosphonate 15.3.

For example, 3-methyl-furo[3,4-b]pyridine-5,7-dione 15.4, (German Patent DE 3707530) is converted, using the procedures described above, into 5-bromo-8-(tert-butyl-dimethyl-silanyloxy)-3-methyl-[1,6]naphthyridine-7-carboxylic acid [1-(3-chloro-4-fluoro-phenyl)-1-methyl-ethyl]-amide 15.5. The product is then coupled, in the presence of tetrakis(triphenylphosphine)palladium(0) and triethylamine, as described in Scheme 3, with a dialkyl phosphite 15.6, to afford, after desilylation of the phenol, the arylphosphonate 15.7.

Using the above procedures, but employing, in place of the bromoamide 15.5, different bromoamides 15.1, the corresponding products 15.3 are obtained. 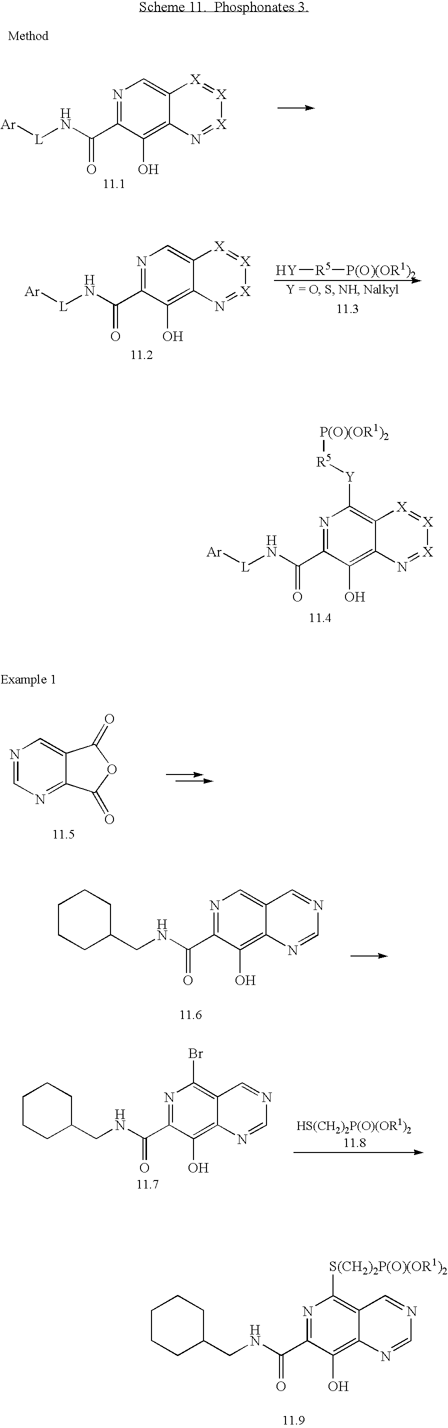 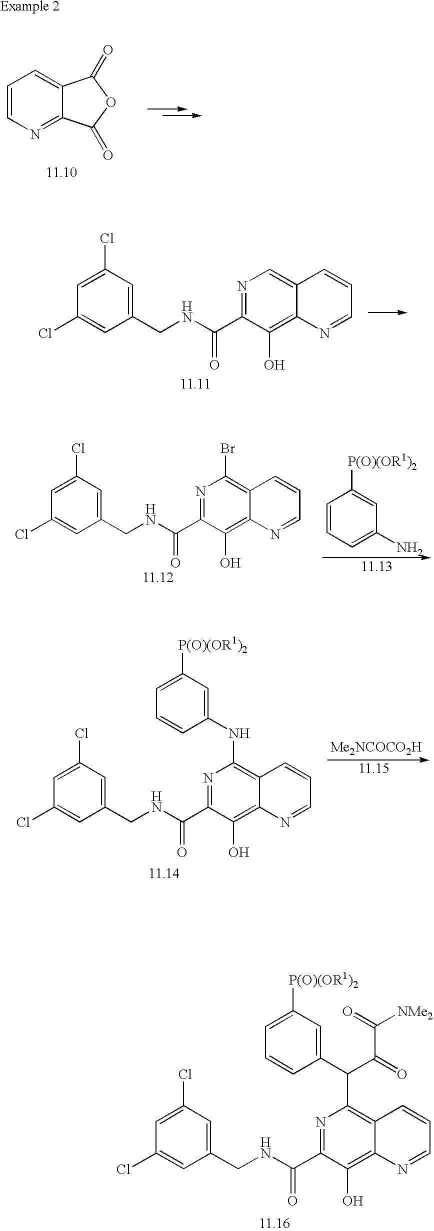 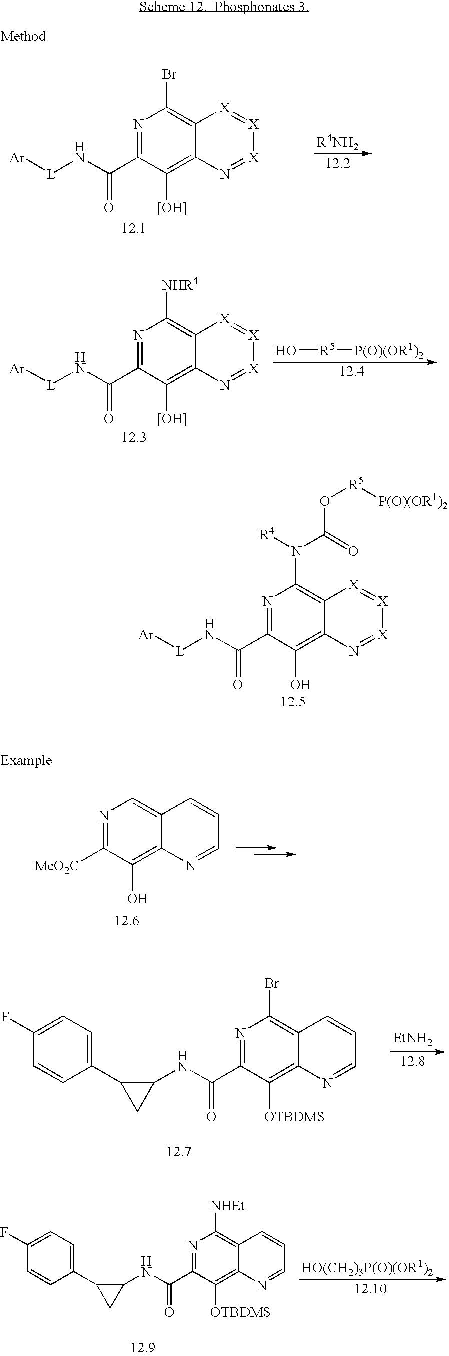 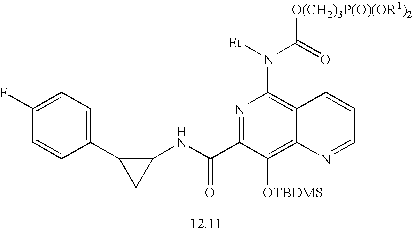 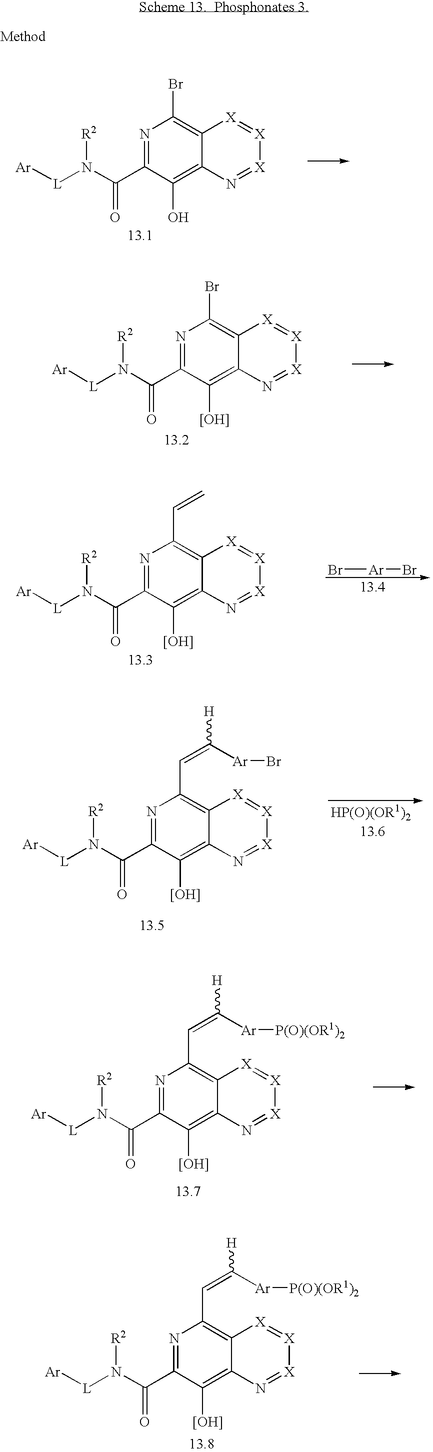 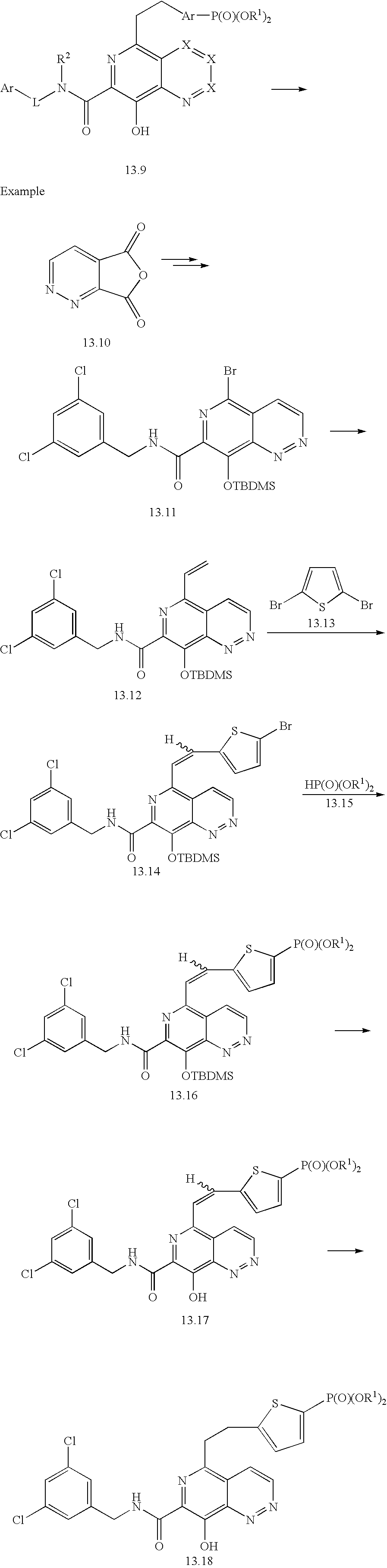 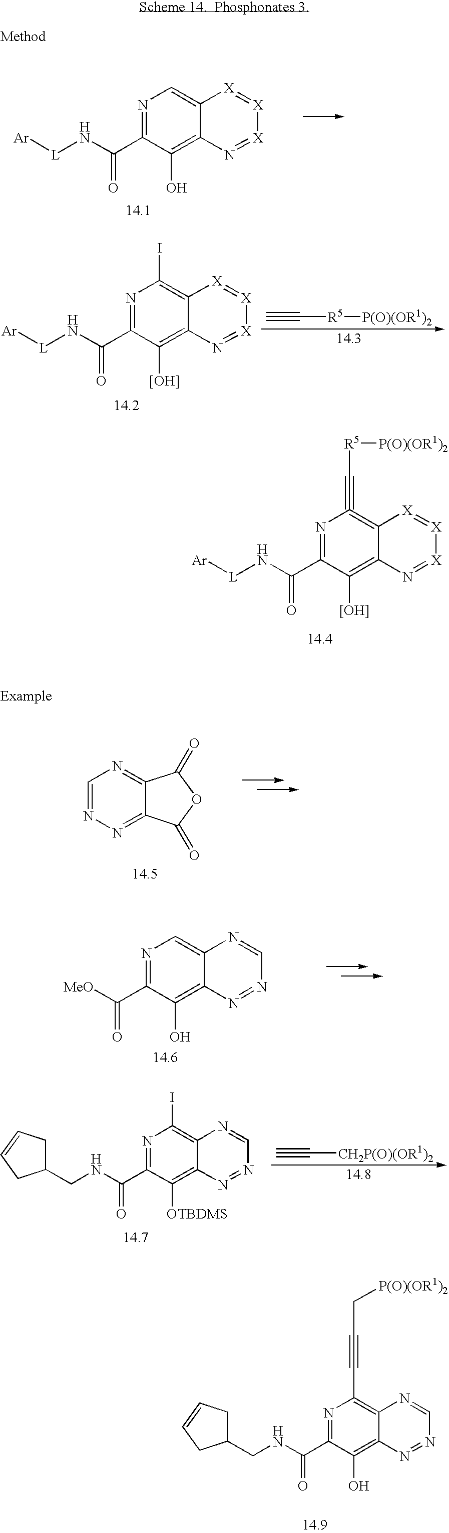 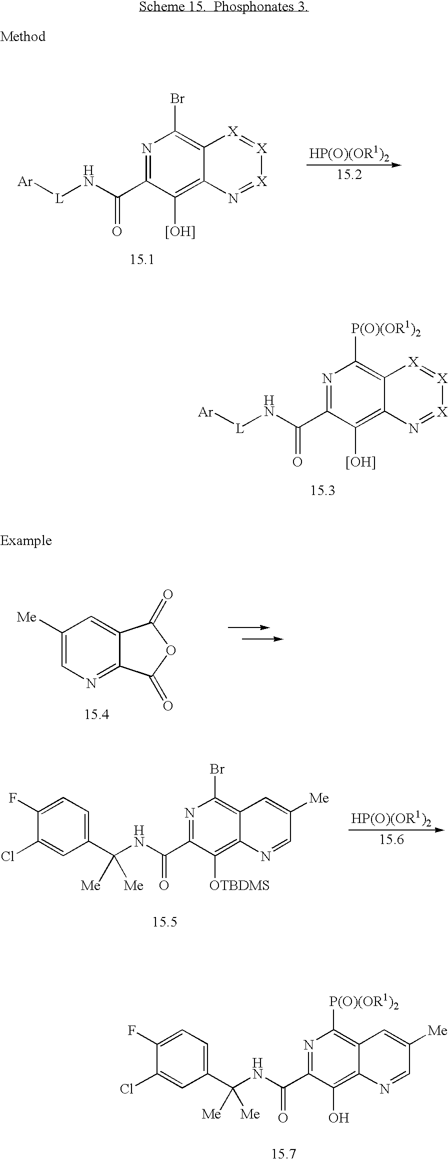
Preparation of the Intermediate Phosphonate Esters 4.

Schemes 16-18 illustrate methods for the preparation of the phosphonate esters 4.

Scheme 16 depicts the preparation of phosphonate esters 4 in which the phosphonate group is attached by means of a variable carbon chain. In this procedure, the phosphonate 16.1, in which the phenolic hydroxyl group is protected, prepared as described in Scheme 11, is reacted with the sulfonyl chloride 16.2 or the sulfonic acid 16.3 to afford after deprotection the sulfonamide 16.4. The reaction between an amine and a sulfonyl chloride, to produce the sulfonamide, is conducted at ambient temperature in an inert solvent such as dichloromethane, in the presence of a tertiary base such as triethylaamine. The reaction between a sulfonic acid and an amine to afford a sulfonamide is conducted in a polar solvent such as dimethylformamide, in the presence of a carbodiimide such as dicyclohexyl carbodiimide, for example as described in Syn., 1976, 339.

For example, the protected amine phosphonate 16.5, prepared by the methods described above, is reacted in dichloromethane solution with one molar equivalent of ethyl sulfonyl chloride 16.6 and triethylamine, to produce, after desilylation, the sulfonamide 16.7.

Using the above procedures, but employing, in place of the amine phosphonate 16.5, different phosphonates 16.1, and/or different sulfonyl chlorides 16.2 or sulfonic acids 16.3, the corresponding products 16.4 are obtained.

Scheme 17 depicts an alternative method for the preparation of phosphonate esters 4 in which the phosphonate group is attached by means of a variable carbon chain. In this procedure, a dialkyl amino-substituted phosphonate 17.1, in which the group R⁵ is as defined in Scheme 4, is reacted with a sulfonyl chloride 17.2 or sulfonic acid 17.3, as described in Scheme 16, to yield the sulfonamide 17.4. The product is then reacted with a bromoamide 17.5, to prepare the displacement product 17.6. The displacement reaction is performed in a basic solvent such as pyridine or quinoline, at from about 80° to reflux temperature, optionally in the presence of a promoter such as copper oxide, as described in WO 0230930 A2 Example 154.

For example, a dialkyl 4-aminophenyl phosphonate 17.7 (Epsilon) is reacted in dichloromethane solution with one molar equivalent of methanesulfonyl chloride 17.8 and triethylamine, to give the sulfonamide 17.9. The product is then reacted in pyridine solution at reflux temperature with 5-bromo-8-hydroxy-pyrido[3,4-b]pyrazine-7-carboxylic acid 4-fluoro-benzylamide 17.10, prepared by the methods described above, and copper oxide, to yield the sulfonamide 17.11.

Using the above procedures, but employing, in place of the amine phosphonate 17.7, different phosphonates 17.1, and/or different sulfonyl chlorides 17.2 or sulfonic acids 17.3, the corresponding products 17.6 are obtained.

Scheme 18 depicts an alternative method for the preparation of phosphonate esters 4 in which the phosphonate group is attached by means of a variable carbon chain. In this procedure, a phenol-protected 5-bromo substituted amide 18.1 is reacted, as described in Scheme 17, with a sulfonamide 18.2, to give the displacement product 18.3. The product is then reacted with a dialkyl bromoalkyl phosphonate 18.4 to afford, after deprotection of the phenol, the alkylated compound 18.5. The alkylation reaction is performed in a polar aprotic solvent such as dimethylformamide or DMPU, at from ambient temperature to about 100°, in the presence of a base such as sodium hydride or lithium hexamethyl disilylazide.

For example, benzoic acid 5-bromo-7-[1-(3-methoxy-phenyl)-1-methyl-ethylcarbamoyl]-[1,6]naphthyridin-8-yl ester 18.6, prepared by the methods described above, is reacted in pyridine solution at reflux temperature with one molar equivalent of propanesulfonamide 18.7 and copper oxide, to afford the sulfonamide 18.8. The product is then reacted in dimethylformamide solution with one molar equivalent of a dialkyl bromoethyl phosphonate 18.9 (Aldrich) and lithium hexamethyl disilylazide, to give after debenzoylation, the sulfonamide phosphonate 18.10. The benzoyl protecting group is removed, for example, by reaction with 1% methanolic sodium hydroxide at ambient temperature, as described in Tet., 26, 803, 1970.

Using the above procedures, but employing, in place of the bromo compound 18.6, different bromo compounds 18.1, and/or different sulfonamides 18.2, and/or different phosphonates 18.4, the corresponding products 18.5 are obtained. 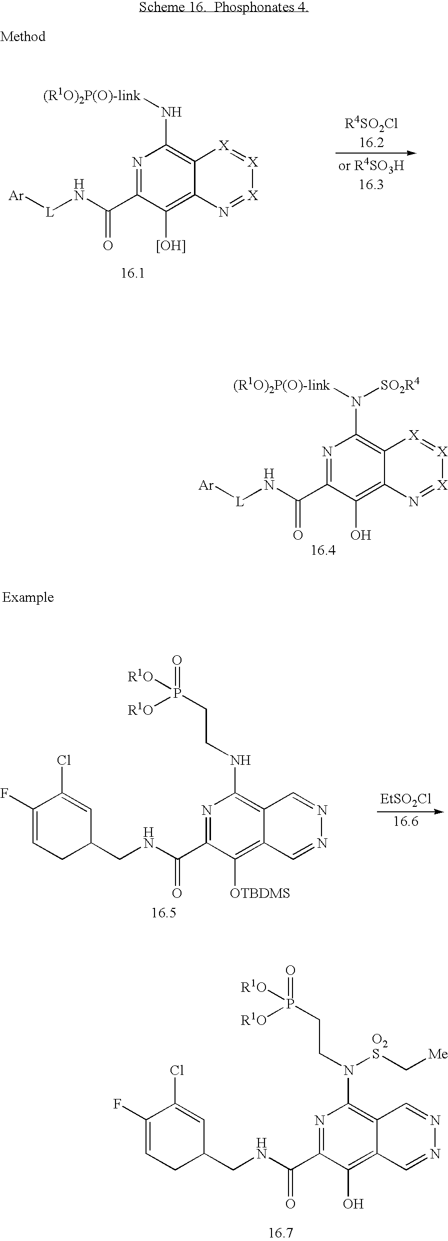 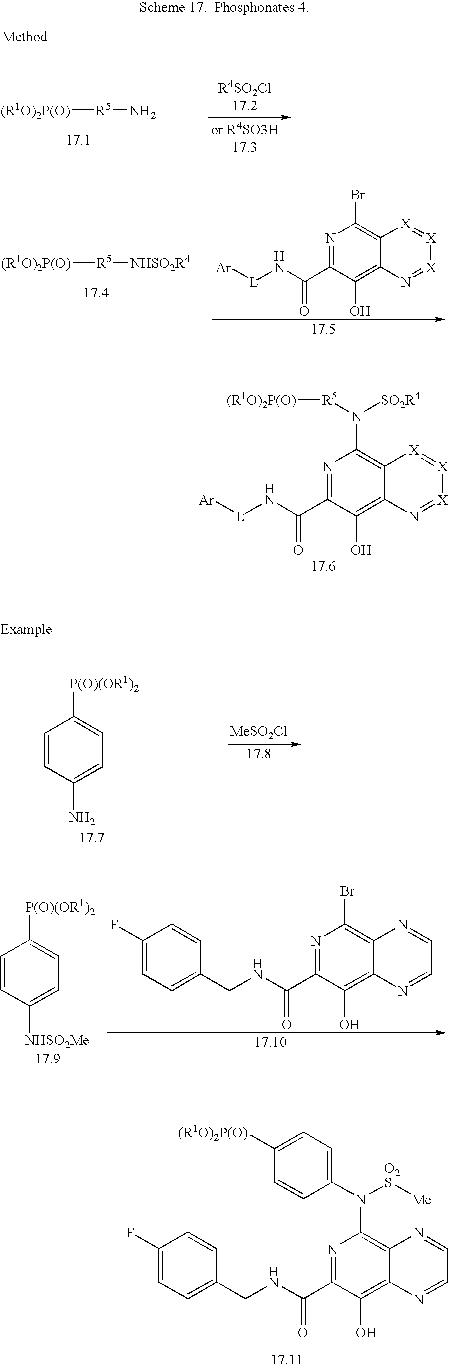 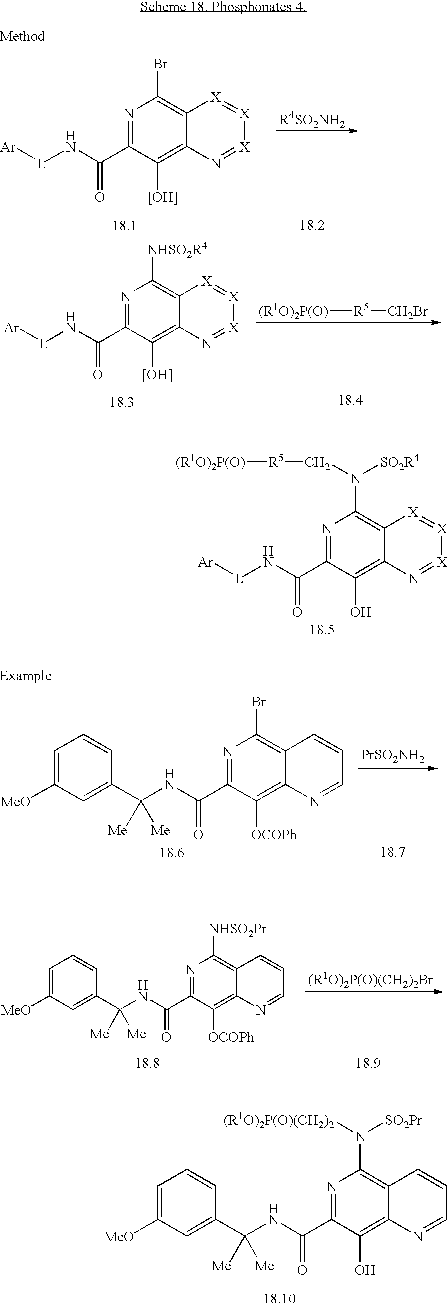
Preparation of the Intermediate Phosphonate Esters 5.

Schemes 19-21 illustrate methods for the preparation of the phosphonate esters 5.

Scheme 19 illustrates the preparation of phosphonates 5 in which the phosphonate group is attached by means of a variable carbon chain. In this procedure, a bromo-substituted sulfonic acid 19.1 is subjected to an Arbuzov reaction with a trialkyl phosphite 19.2 to give the phosphonate 19.3. The Arbuzov reaction is performed by heating the bromo compound with an excess of the trialkyl phosphite at from 100° to 150°, as described in Handb. Organophosphorus Chem., 1992, 115-72. The resulting phosphonate is then reacted with an amine 19.4, either directly, in the presence of a carbodiimide, or by initial conversion to the sulfonyl chloride, as described in Scheme 16, to afford, after deprotection of the phenolic hydroxyl group, the sulfonamide 19.5.

For example, 3-bromopropanesulfonic acid 19.6 (Sigma) is heated at 130′ with a trialkyl phosphite 19.7 to give the phosphonate 19.8. The product is then reacted in DMPU solution with 8-(tert-butyl-dimethyl-silanyloxy)-5-ethylamino-[1,6]naphthyridine-7-carboxylic acid 4-fluoro-benzylamide 19.9, prepared by the methods described above, in the presence of dicyclohexylcarbodiimide, to give, after desilylation, by reaction with tetrabutylammonium fluoride in tetrahydrofuran, the sulfonamide 19.10.

Using the above procedures, but employing, in place of the bromo sulfonic acid 19.6, different bromosulfonic acids 19.1, and/or different amines 19.4, the corresponding products 19.5 are obtained.

Scheme 20 illustrates the preparation of phosphonates 5 in which the phosphonate group is attached by means of a saturated or unsaturated carbon chain and an aromatic or heteroaromatic group. In this procedure, a vinyl-substituted sulfonic acid 20.1 is coupled, in a palladium-catalyzed Heck reaction, as described in Scheme 4, with a dibromoaromatic or heteroaromatic compound 20.2, to yield the sulfonic acid 20.3. The product is then coupled, in the presence of a palladium catalyst, as described in Scheme 3, with a dialkyl phosphite HP(O)(OR¹)₂, to give the phosphonate 20.4. The latter compound is then reacted, as described above, with an amine 20.5, either directly, in the presence of a carbodiimide, or by initial conversion to the sulfonyl chloride, as described in Scheme 16, to afford, after deprotection of the phenolic hydroxyl group, the sulfonamide 20.6. Optionally, the double bond is reduced, either catalytically or chemically, as described in Scheme 4, to afford the saturated analog 20.7.

For example, vinylsulfonic acid 20.8 (Sigma) is coupled, in dioxan solution, in the presence of tetrakis(triphenylphosphine)palladium (0) and potassium carbonate, with 2,5-dibromothiophene 20.9, to form the coupled product 20.10. The product is then reacted in toluene solution at 100° with a dialkyl phosphite 20.11, triethylamine and a catalytic amount of tetrakis(triphenylphosphine)palladium (0), to produce the phosphonate 20.12. This material is then reacted, in dimethylformamide solution at ambient temperature, as described above, with 8-(tert-butyl-dimethyl-silanyloxy)-5-cyclopropylamino-pyrido[4,3-d]pyrimidine-7-carboxylic acid 4-fluoro-benzylamide 20.13, prepared by the methods described above, in the presence of dicyclohexylcarbodiimide, to give, after desilylation, using tetrabutylammonium fluoride, the sulfonamide 20.14. Hydrogenation of the double bond, for example using 5% palladium on carbon as catalyst, then yields the saturated analog 20.15.

Using the above procedures, but employing, in place of the sulfonic acid 20.8, different sulfonic acids 20.1, and/or different dibromoaromatic compounds 20.2, and/or different amines 20.5, the corresponding products 20.6 and 20.7 are obtained.

Scheme 21 illustrates the preparation of phosphonates 5 in which the phosphonate group is attached by means of a variable carbon chain. In this procedure, an aliphatic bromo-substituted sulfonic acid 21.1 is subjected to an Arbuzov reaction with a trialkyl phosphite, as described in Scheme 19, to give the phosphonate 21.2. Alternatively, an aryl bromosulfonic acid 21.1 is coupled, as described in Scheme 3, with a dialkyl phosphite, to give the phosphonate 21.2. The product is then reacted with an amine 21.3 to afford the sulfonamide 21.4. The latter compound is then reacted, as described in Scheme 17, with a bromoamide 21.5, to give the displacement product 21.6.

For example, 4-bromobenzenesulfonic acid 21.7 is reacted, as described in Scheme 20, with a dialkyl phosphite to form the phosphonate 21.8. The product is then reacted with phosphoryl chloride to afford the corresponding sulfonyl chloride, and the latter compound is reacted, in dichloromethane solution, in the presence of triethylamine, with 2-methoxyethylamine 21.9, to yield the sulfonamide 21.10. This material is then reacted, in pyridine solution at reflux temperature, with 5-bromo-8-hydroxy-[1,6]naphthyridine-7-carboxylic acid 4-fluoro-benzylamide 21.11, prepared by the methods described above, and copper oxide, to give the sulfonamide 21.12.

Using the above procedures, but employing, in place of the sulfonic acid 21.7, different sulfonic acids 21.1, and/or different amines 21.3, and/or different bromo compounds 21.5, the corresponding products 21.6 are obtained.

Preparation of the Intermediate Phosphonate Esters 6.

Schemes 22-24 illustrate methods for the preparation of the phosphonate esters 6.

Scheme 22 depicts the preparation of phosphonates 6 in which the phosphonate group is attached by means of an amide linkage and a variable carbon chain. In this procedure, a cyclic sulfonamide 22.1, incorporating a secondary amine, is coupled, as described in Scheme 5, with a dialkyl carboxy-substituted phosphonate 22.2 to produce the amide 22.3. The product is then reacted with a bromoamide 22.4 to afford the displacement product 22.5.

Alternatively, the cyclic sulfonamide 22.1 is protected to give the analog 22.6. Sulfonamides are protected, for example, by conversion into the N-acyloxymethyl derivatives, such as the pivalyloxymethyl derivative or the benzoyloxymethyl derivative, by reaction with the corresponding acyloxymethyl chloride in the presence of dimethylaminopyridine, as described in Bioorg. Med. Chem. Lett., 1995, 5, 937, or by conversion into the carbamate derivative, for example the tert. butyl carbamate, by reaction with an alkyl, aryl or aralkyl chloroformate, in the presence of a base such as triethylamine, as described in Tet. Lett., 1994, 35, 379. The protected sulfonamide is reacted with a dialkyl bromoalkyl phosphonate 22.7 to form the alkylated product 22.8. The alkylation reaction is effected as described in Scheme 8. The product is then deprotected to yield the sulfonamide 22.9. Deprotection of pivalyloxymethyl amides is effected by treatment with trifluoroacetic acid; deprotection of benzyloxymethyl amides is effected by catalytic hydrogenation, as described in Protective Groups in Organic Synthesis, by T. W. Greene and P. G. M. Wuts, Wiley, Second Edition 1990, p. 398. Sulfonamide carbamates, for example the tert. butyl carbamate, are deprotected by treatment with trifluoroacetic acid. The sulfonamide 22.9 is then reacted with the bromoamide 22.10 to give the displacement product 22.11.

For example, [1,2,5]thiadiazepane 1,1-dioxide 22.11A (WO 0230930 A2 p. 321) is reacted in dioxan solution with equimolar amounts of a dialkyl 3-carboxypropyl phosphonate 23.12, (Epsilon) and dicyclohexyl carbodiimide, to produce the amide 22.13. This material is reacted in pyridine solution at reflux temperature with 5-bromo-8-hydroxy-[1,6]naphthyridine-7-carboxylic acid 4-fluoro-benzylamide 22.14, prepared by the methods described above, and copper oxide, to afford the displacement product 22.15.

As a further example, the sulfonamide 22.11A is reacted in dichloromethane with one molar equivalent of t-Boc anhydride, triethylamine and dimethylaminopyridine, to give 1,1-dioxo-[1,2,5]thiadiazepane-2-carboxylic acid tert-butyl ester 22.16. The product is then reacted at ambient temperature in dimethylformamide solution with a dialkyl 4-bromomethyl benzyl phosphonate 22.17, (Tet., 1998, 54, 9341) and potassium carbonate, to yield the alkylation product 22.18. The BOC group is removed by treatment with trifluoroacetic acid to give the sulfonamide 22.19, and this material is reacted, as described above, with 5-bromo-8-hydroxy-[1,6]naphthyridine-7-carboxylic acid 3-fluoro-benzylamide 22.20, prepared by the methods described above, to afford the displacement product 22.21.

Using the above procedures, but employing, in place of the sulfonamide 22.11A, different sulfonamides 22.1, and/or different carboxylic acids 22.2 or alkyl bromides 22.7, and/or different bromides 22.4, the corresponding products 22.5 and 22.11 are obtained.

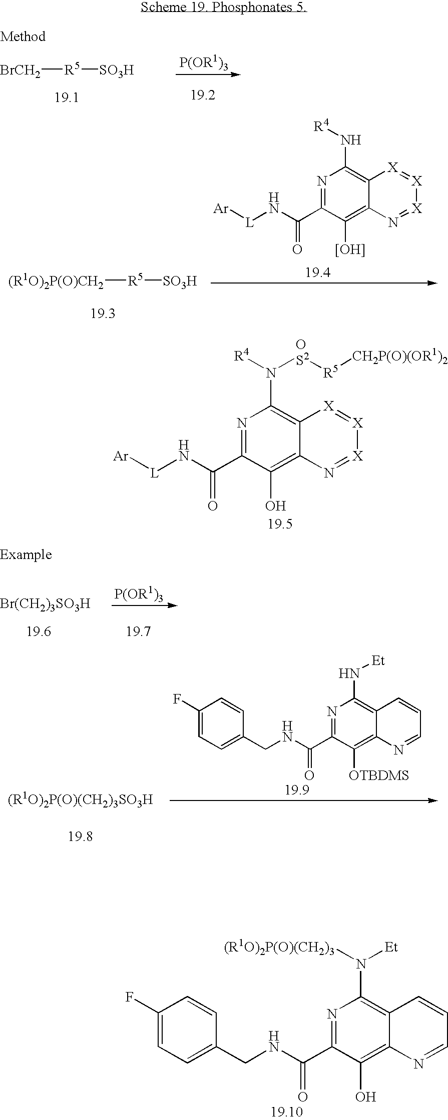

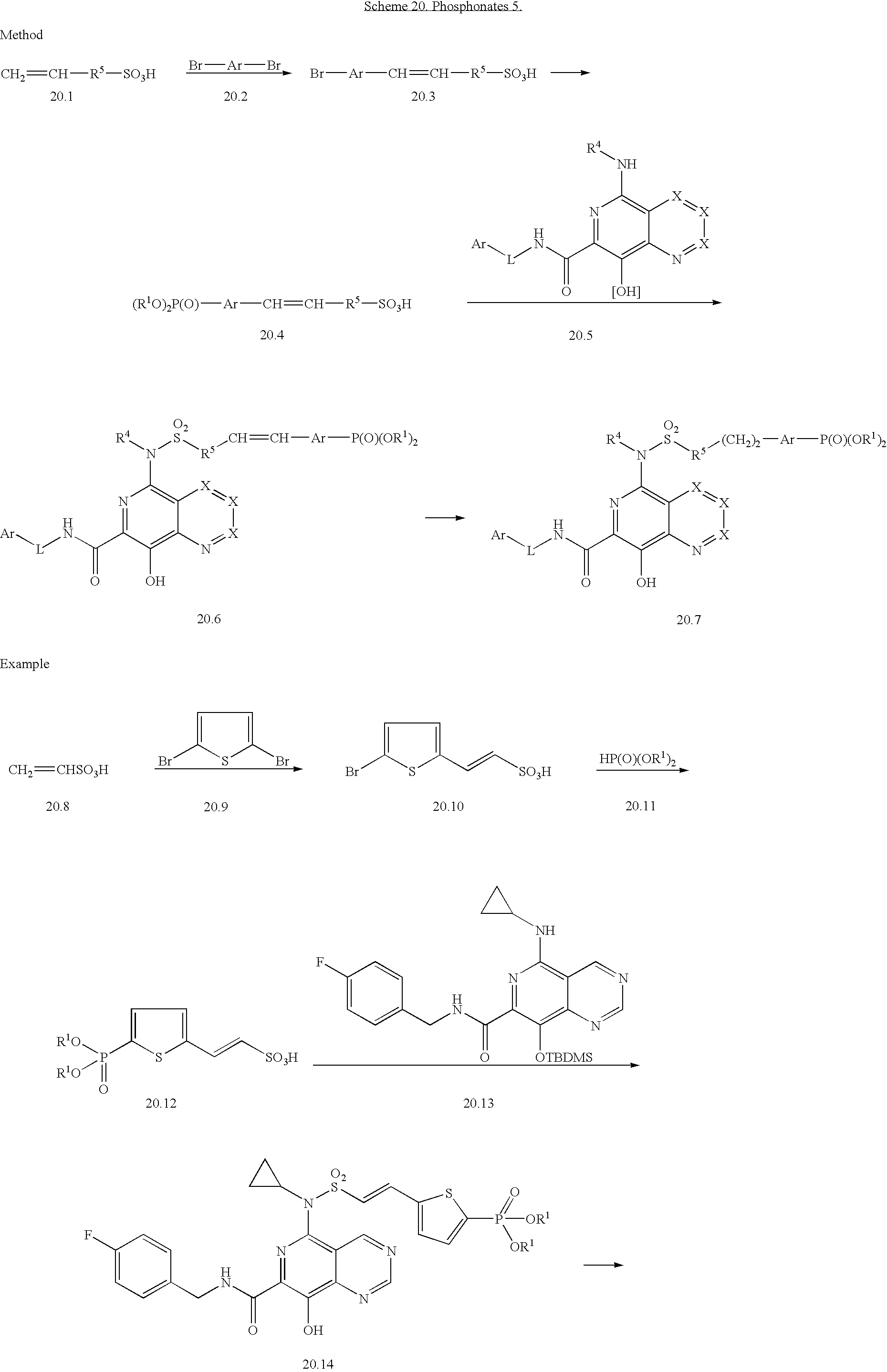

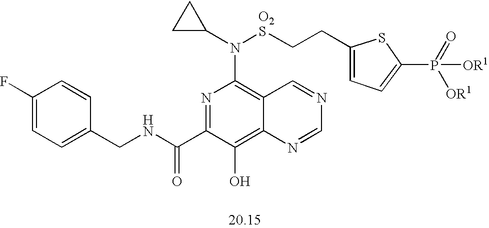

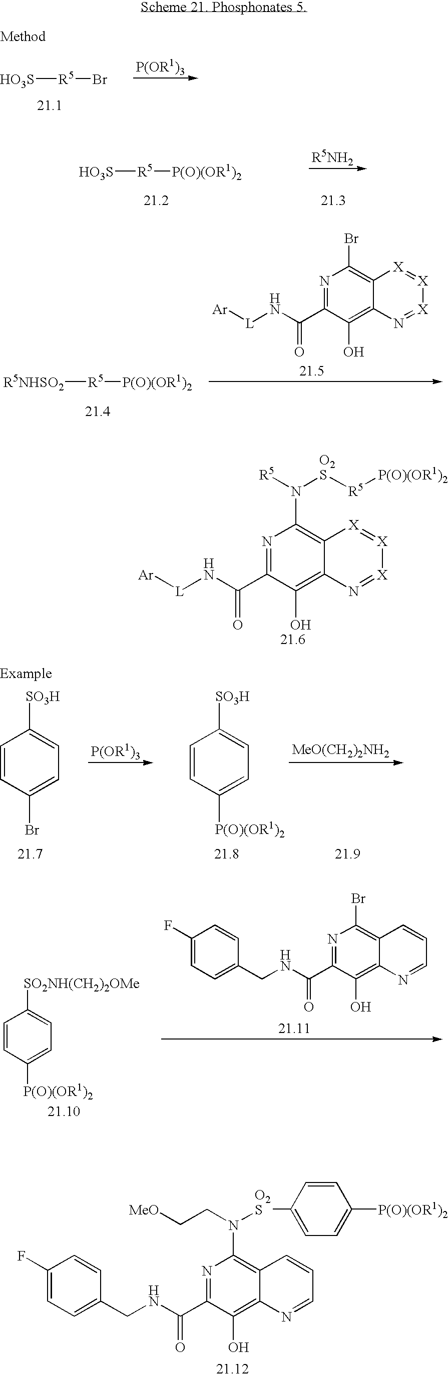

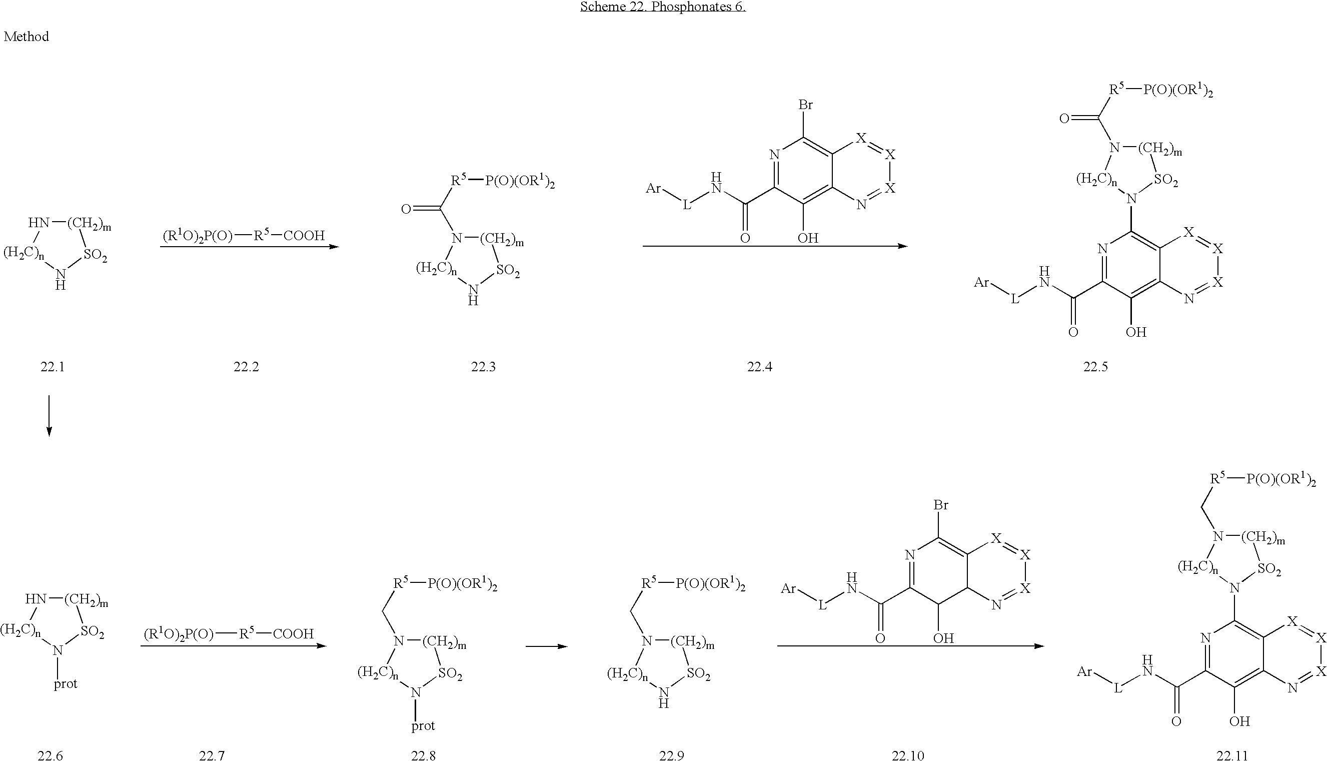

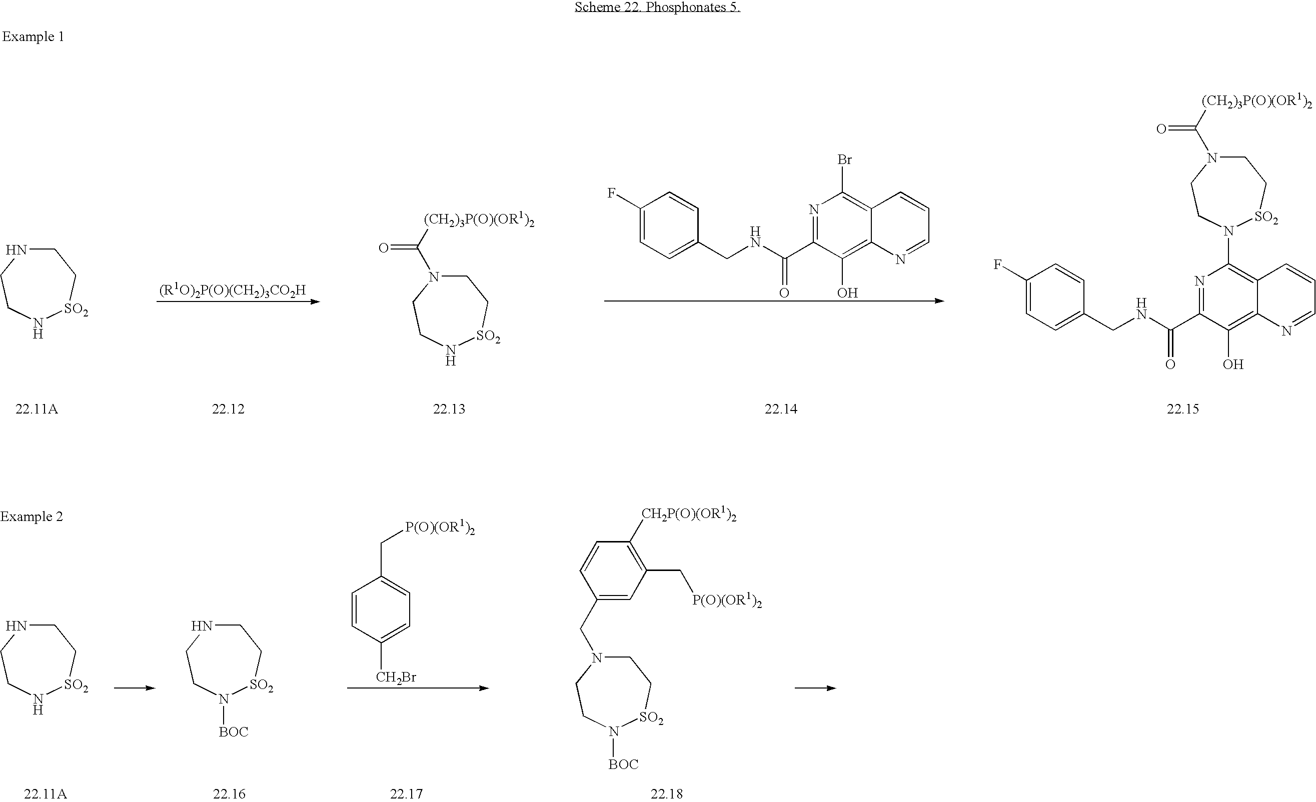

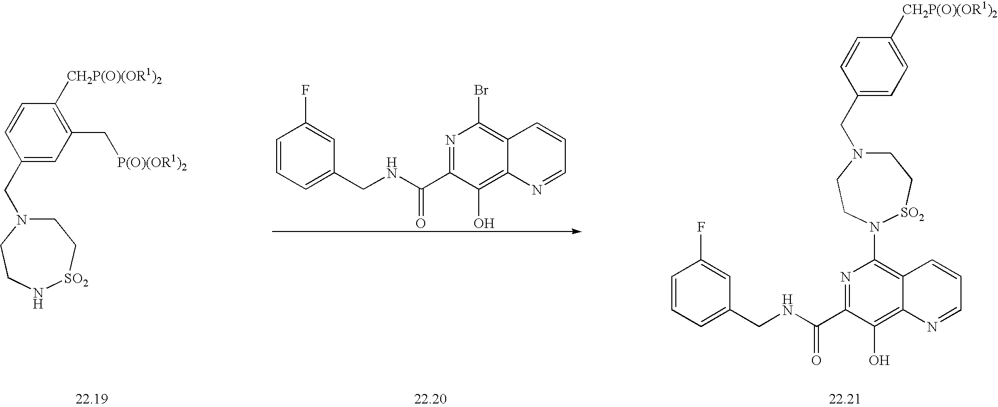

Scheme 23 depicts the preparation of phosphonates 6 in which the phosphonate group is attached by means of an aryl or heteroaryl group. In this procedure, a bromoaryl-substituted cyclic sulfonamide, prepared as described in J. Org. Chem., 1991, 56, 3549, from the corresponding bromoaryl or bromoheteroaryl acetic acid and a vinyl sulfonic ester, is coupled, as described in Scheme 3, with a dialkyl phosphite to afford the phosphonate 23.2. The product is then reacted, as described above, with a bromoamide 23.3 to yield the displacement product 23.4.

For example, 4-(4-bromo-phenyl)-[1,2]thiazinane 1,1-dioxide 23.5 (J. Org. Chem., 1991, 56, 3549) is reacted in dimethylformamide solution with a dialkyl phosphite 23.6 and tetrakis(triphenylphosphine)palladium(0), to give the phosphonate 23.7. The product is then reacted with 5-bromo-8-hydroxy-[1,6]naphthyridine-7-carboxylic acid (5-fluoro-indan-1-yl)-amide 23.8, prepared by the methods described above, to give the phosphonate 23.9.

Using the above procedures, but employing, in place of the sulfonamide 23.5, different sulfonamides 23.1, and/or different bromo compounds 23.3, the corresponding products 23.4 are obtained.

Scheme 24 depicts the preparation of phosphonates 6 in which the phosphonate group is attached by means of an amide linkage. In this procedure, a carboxy-substituted cyclic sulfonamide 24.1 is coupled with an amino-substituted dialkyl phosphonate 24.2, as described in Scheme 5, to give the amide 24.3. The product is then reacted with the bromoamide 24.4 to afford the displacement product 24.5.

For example, 1,1-dioxo-[1,2]thiazinane-3-carboxylic acid 24.6 (Izvest. Akad. Nauk. SSSR Ser. Khim., 1964, 9, 1615) is reacted in dimethylformamide solution with equimolar amounts of an amino-substituted butyl phosphonate 24.7 (Acros) and dicyclohexylcarbodiimide, to afford the amide 24.8. The latter compound is then condensed with 5-bromo-8-hydroxy-[1,6]naphthyridine-7-carboxylic acid [1-(3-chloro-4-fluoro-phenyl)-ethyl]-amide 24.9, prepared by the methods described above, to give the product 24.10.

Using the above procedures, but employing, in place of the sulfonamide 24.6, different sulfonamides 24.1, and/or different bromo compounds 24.4, the corresponding products 24.5 are obtained. 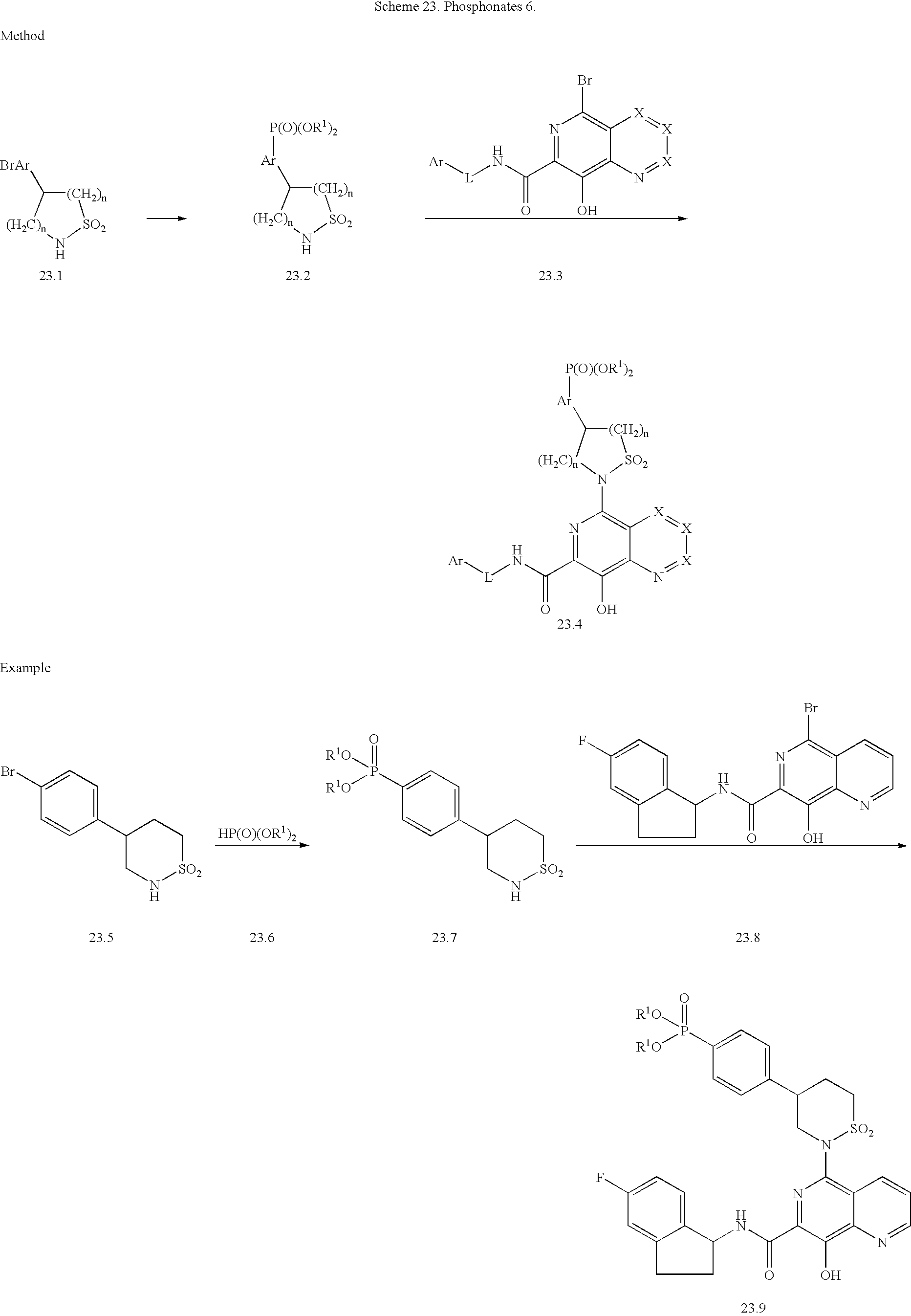 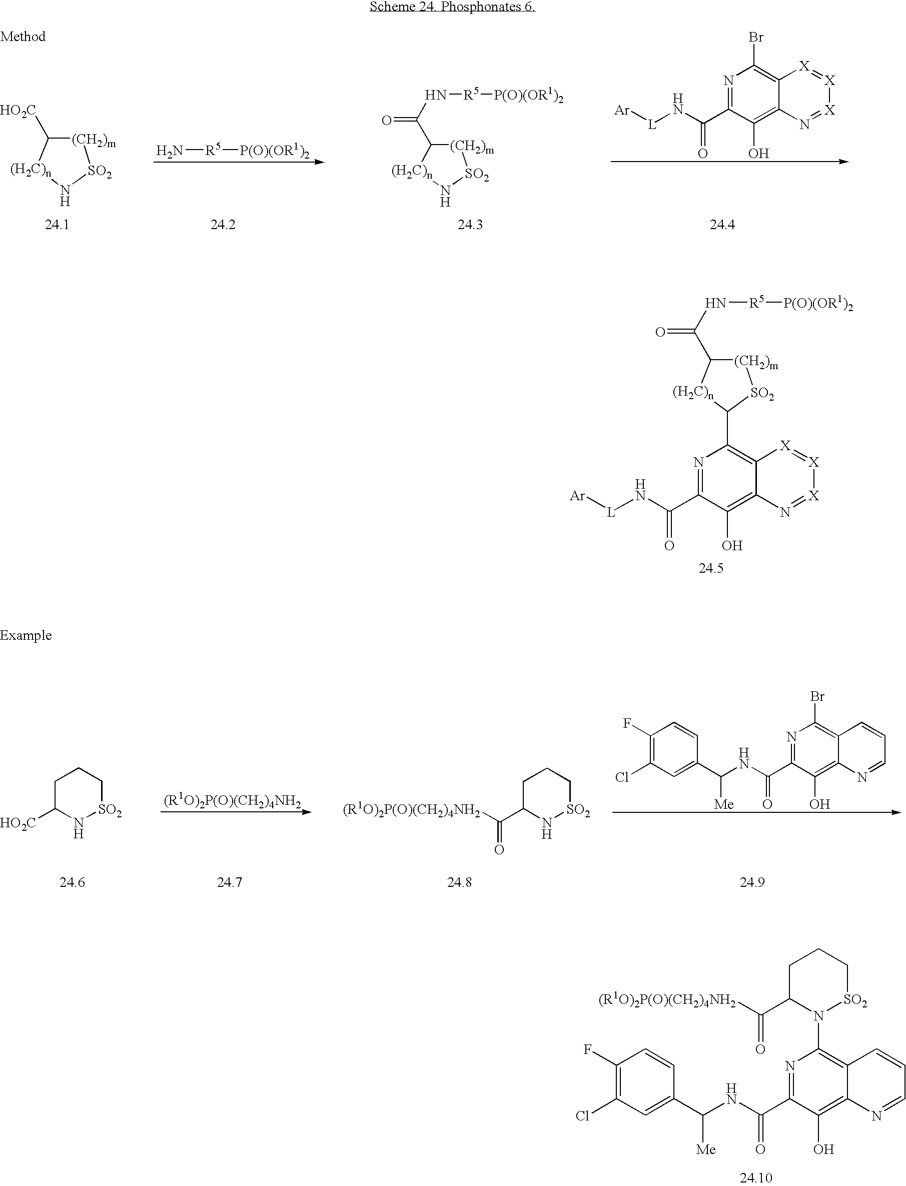
Preparation of the Intermediate Phosphonate Esters 7.

Schemes 25-27 illustrate methods for the preparation of the phosphonate esters 7.

Scheme 25 illustrates the preparation of phosphonate esters 7 in which the phosphonate is attached by means of a carbon link or a variable carbon chain incorporating a heteroatom. In this procedure, a methyl-substituted cyclic anhydride 25.1 is converted, as described in Schemes 1 and 2, into the bicyclic amide 25.2, in which the phenolic hydroxyl group is protected. The compound is reacted with a free radical brominating agent such as N-bromosuccinimide to prepare the bromomethyl derivative 25.3. The benzylic bromination reaction is performed at reflux temperature in an inert organic solvent such as hexachloroethane or ethyl acetate, optionally in the presence of an initiator such as dibenzoyl peroxide. The bromomethyl compound 25.3 is then reacted with a trialkyl phosphite in an Arbuzov reaction, as described in Scheme 19, to give, after deprotection of the phenolic hydroxyl group, the phosphonate 25.4.

Alternatively, the benzylic bromide 25.3 is reacted with a dialkyl hydroxy, mercapto or amino-substituted phosphonate 25.5, to afford, after deprotection of the phenolic hydroxyl group, the displacement product 25.6. The displacement reaction is effected at from ambient temperature to about 100°, in a polar organic solvent such as dimethylformamide or DMPU, in the presence of a suitable base such as sodium hydride or lithium hexamethyldisilazide, for instances in which Y is O, or cesium carbonate or triethylamine for instances in which Y is S or N.

For example, 4-methyl-furo[3,4-b]pyridine-5,7-dione 25.7, (J. Org. Chem., 1961, 26, 808) is converted, using the methods described above, into 5-(1,1-dioxo-isothiazolidin-2-yl)-4-methyl-8-triisopropylsilanyloxy-[1,6]naphthyridine-7-carboxylic acid 4-fluoro-benzylamide 25.8. The compound is then reacted with one molar equivalent of N-bromosuccinimide in ethyl acetate at reflux, to afford the bromomethyl analog 25.9. This product is reacted with a dialkyl hydroxyethyl phosphonate 25.11 (Epsilon) and sodium hydride in dimethylformamide at 80°, to yield, after desilylation, the phosphonate 25.12. Alternatively, the bromomethyl compound 25.9 is reacted at 120° with a trialkyl phosphite, to obtain, after desilylation, the phosphonate 25.10.

Using the above procedures, but employing, in place of the anhydride 25.7, different anhydrides 25.1, and/or different phosphonates 25.5, the corresponding products 25.4 and 25.6 are obtained.

Scheme 26 illustrates the preparation of phosphonate esters 7 in which the phosphonate is attached by means of an aminomethyl linkage. In this procedure, a bromomethyl-substituted bicyclic amide 25.3, prepared as described in Scheme 25, is oxidized to the corresponding aldehyde 26.1. The oxidation of halomethyl compounds to aldehydes is described, for example, in Comprehensive Organic Transformations, by R. C. Larock, VCH, 1989, p. 599ff. The transformation is effected by treatment with dimethylsulfoxide and base, optionally in the presence of a silver salt, or by reaction with trimethylamine N-oxide or hexamethylene tetramine. The aldehyde 26.1 is then reacted with a dialkyl amino-substituted phosphonate 26.2 in a reductive amination reaction, as described in Scheme 9, to yield, after deprotection of the phenolic hydroxyl group, the aminomethyl product 26.3.

For example, 4-bromomethyl-5-(methanesulfonyl-methyl-amino)-8-triisopropylsilanyloxy-[1,6]naphthyridine-7-carboxylic acid 3,5-dichloro-benzylamide 26.4, prepared from the anhydride 25.7, using the methods described in Scheme 25, is reacted with dimethylsulfoxide and 2,4,6-collidine at 90°, as described in J. Org. Chem., 51, 1264, 1986, to afford the aldehyde 26.5. The product is then reacted with one molar equivalent of a dialkyl aminoethyl phosphonate 26.6 (Epsilon) and sodium triacetoxyborohydride to produce, after desilylation, the phosphonate 26.7.

Using the above procedures, but employing, in place of the bromomethyl compound 26.4, different bromomethyl compounds 25.3, and/or different phosphonates 26.2, the corresponding products 26.3 are obtained.

Scheme 27 illustrates the preparation of phosphonate esters 7 in which the phosphonate is attached by means of an amide linkage. In this procedure, an aldehyde 26.1 (Scheme 26) is oxidized to the corresponding carboxylic acid 27.1. The conversion of aldehydes to the corresponding carboxylic acids is described in Comprehensive Organic Transformations, by R. C. Larock, VCH, 1989, p. 838. The reaction is effected by the use of various oxidizing agents such as, for example, potassium permanganate, ruthenium tetroxide, silver oxide or sodium chlorite. The resultant carboxylic acid 27.1 is then coupled, as described in Scheme 5, with a dialkyl amino-substituted phosphonate 27.2, to yield, after deprotection of the phenolic hydroxyl group, the amide 27.3.

For example, the anhydride 27.4 is converted, as described above, and in Schemes 25 and 26, into N-[7-(2-cyclohex-3-enyl-ethylcarbamoyl)-4-formyl-8-triisopropylsilanyloxy-[1,6]naphthyridin-5-yl]-N,N′,N′-trimethyl-oxalamide 27.5. The aldehyde is then reacted with silver oxide in aqueous sodium hydroxide, as described in Org. Syn. Coll. Vol. 4, 919, 1963, to afford the carboxylic acid 27.6. The latter compound is then reacted in dioxan solution at ambient temperature with equimolar amounts of a dialkyl aminomethyl phosphonate 27.7 (Interchim) and dicyclohexylcarbodiimide, to give, after desilylation, the amide phosphonate 27.8.

Using the above procedures, but employing, in place of the aldehyde 27.5, different aldehydes 26.1, and/or different phosphonates 27.2, the corresponding products 27.3 are obtained. 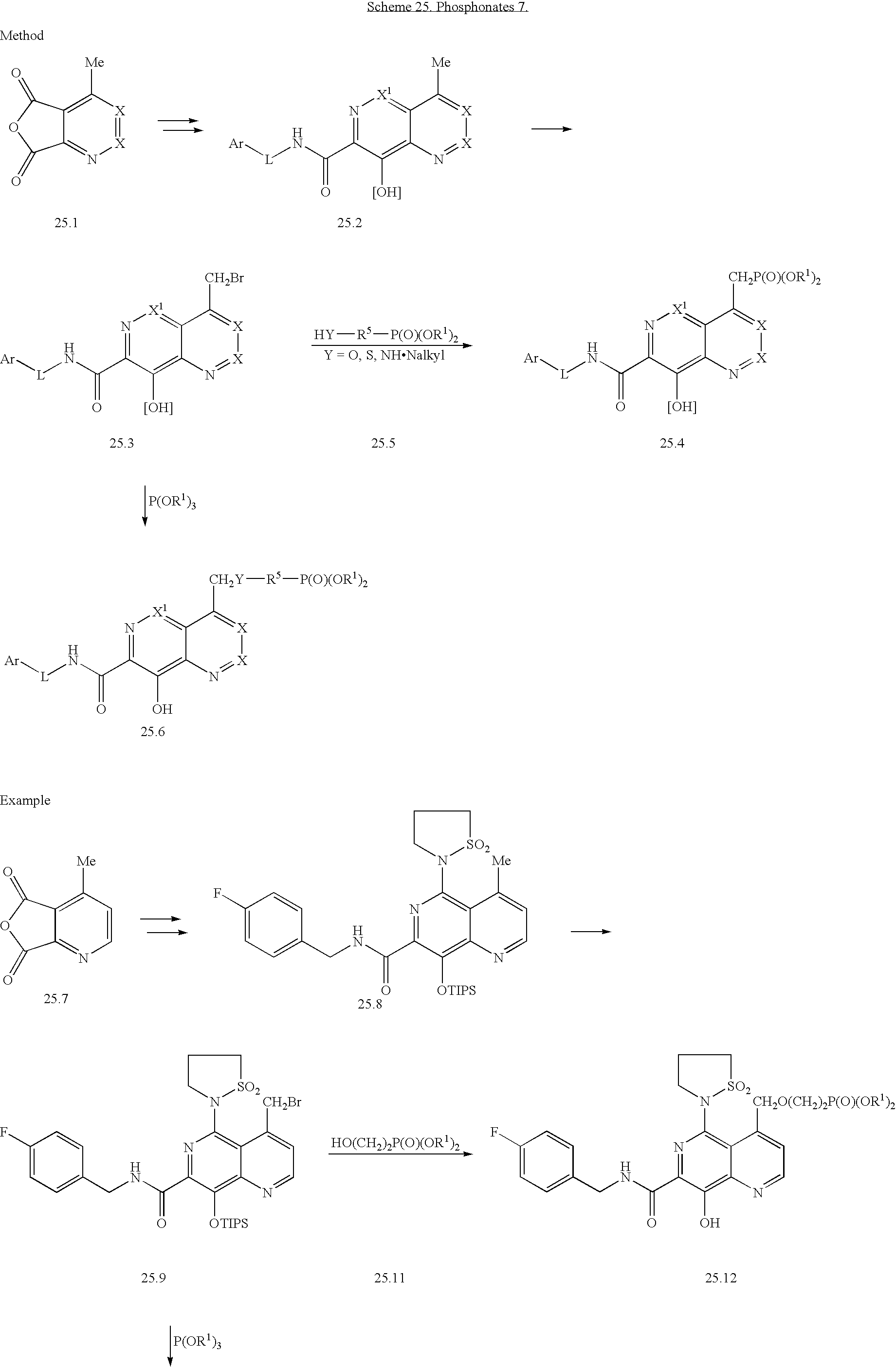 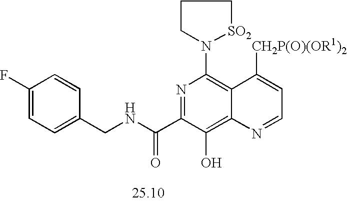 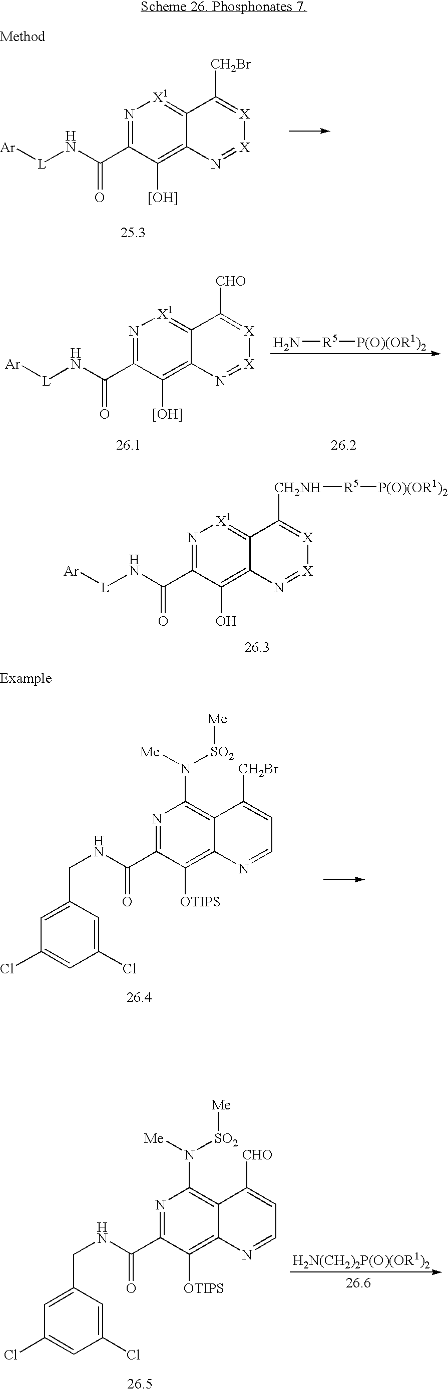 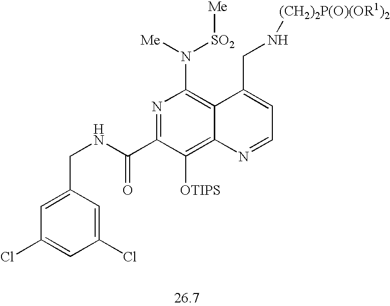 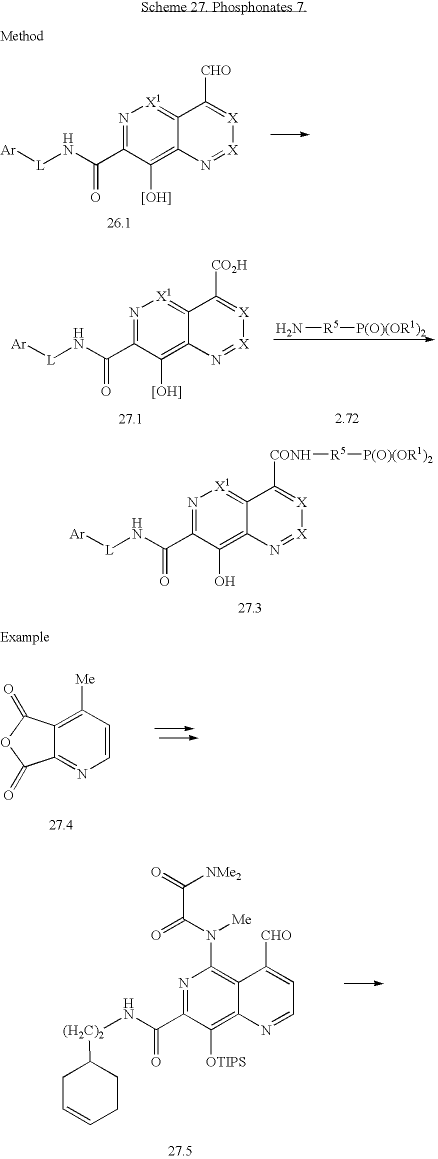 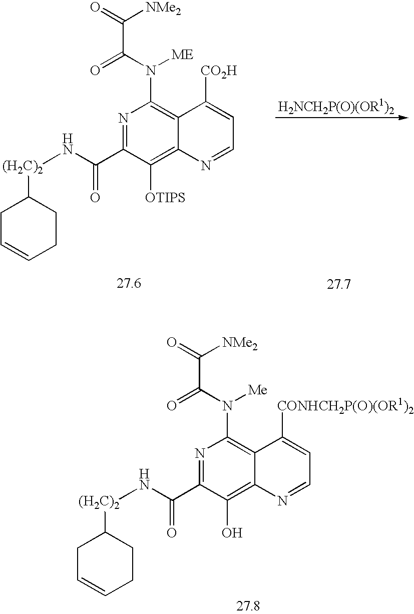
Preparation of the Intermediate Phosphonate Esters 8.

Schemes 28 and 29 illustrate methods for the preparation of the phosphonate esters 8.

Scheme 28 illustrates the preparation of phosphonate esters 8 in which the phosphonate is attached by means of a heteroatom O or S and a variable carbon link. In this procedure, the hydroxyl group of a hydroxy-substituted cyclic anhydride 28.1 is protected to afford the compound 28.2. The product is then converted, as described in Scheme 1, into the bicyclic ester 28.3, in which the phenol protecting groups are different. The original phenolic hydroxyl group is then deprotected to yield the phenol 28.4, and the product is subjected to a Mitsunobu reaction, as described in Scheme 7, with a dialkyl hydroxy or mercapto-substituted phosphonate 28.8, to produce the ether or thioether phosphonate 28.9. This material is then reacted, as described in Scheme 3, with the amine ArLNR²H, to give after deprotection of the phenolic hydroxyl group, the amide 28.10.

Alternatively, the phenol 28.4 is reacted with a dialkyl bromoalkyl-substituted phosphonate 28.5, as described in Scheme 6, to yield the ether 28.6. The latter compound is then transformed, as described above, into the amide 28.7.

For example, 3-hydroxy-furo[3,4-b]pyridine-5,7-dione 28.11 (German Patent 4343923) is reacted in tetrahydrofuran solution at 50° with 4-methoxybenzyl bromide and potassium carbonate, to give the 4-methoxybenzyl ether 28.12. The product is then converted, as described above, into the silyl-protected bicyclic ester 28.13. The 4-methoxybenzyl ether is then removed by reaction with dichlorodicyanobenzoquinone in dichloromethane at ambient temperature, as described in Tet. Lett., 27, 3651, 1986, to give the phenol 28.14. The product is then reacted in tetrahydrofuran solution with a dialkyl bromomethyl phosphonate 29.15 (Lancaster) and potassium carbonate, to produce the phosphonate 28.16; the product is then converted, by desilylation, amide formation, bromination, reaction with methylamine and carbamate formation, using the procedures described above, into the hydroxyamide 28.17.

Alternatively, the phenol 28.14 is reacted in tetrahydrofuran solution with one molar equivalent of a dialkyl 2-mercaptoethyl phosphonate 28.18 (Zh. Obschei. Khim., 1973, 43, 2364), diethylazodicarboxylate and triphenylphosphine, to prepare the thioether phosphonate 28.19. The product is then converted, as described above, into the amide 28.20.

Using the above procedures, but employing, in place of the anhydride 28.11, different anhydrides 28.1, and/or different phosphonates 28.5 or 28.8, the corresponding products 28.7 and 28.10 are obtained.

Scheme 29 illustrates the preparation of phosphonate esters 8 in which the phosphonate is attached either directly, or by means of a saturated or unsaturated carbon chain. In this procedure, a bromo-substituted anhydride 29.1 is converted, as described above, into the phenol-protected amide 29.2. The product is then subjected to a Heck coupling reaction, in the presence of a palladium (0) catalyst, as described in Scheme 4, with a dialkyl alkenyl phosphonate 29.3, to afford, after deprotection of the phenol, the phosphonate 29.4. Optionally, the olefinic bond is reduced, as described in Scheme 4, to yield the saturated analog 29.5.

Alternatively, the bromo-substituted amide 29.2 is coupled, as described in Scheme 3, with a dialkyl phosphite, in the presence of a palladium (0) catalyst, to generate, after deprotection of the phenolic hydroxyl group, the amide phosphonate 29.6.

For example, 3-bromo-furo[3,4-b]pyridine-5,7-dione 29.7, (Bioconjugate Chem., 2003, 14, 629) is converted, using the methods described above, into 3-bromo-5-(1,1-dioxo-[1,2]thiazinan-2-yl)-8-triisopropylsilanyloxy-[1,6]naphthyridine-7-carboxylic acid 4-trifluoromethyl-benzylamide 29.8. This compound is then reacted, in dimethylformamide solution at 80°, with one molar equivalent of a dialkyl vinyl phosphonate 29.9, (Aldrich), triethylamine and a catalytic amount of tetrakis(triphenylphosphine)palladium(0) to yield, after desilylation, the unsaturated phosphonate 29.10. The product is then reacted with diimide, prepared by basic hydrolysis of diethyl azodicarboxylate, as described in Angew. Chem. Int. Ed., 4,271, 1965, to yield the saturated product 29.11.

Alternatively, the bromo compound 29.8 is reacted in toluene solution at ca. 100°, with one molar equivalent of a dialkyl phosphite 29.2, triethylamine and 3 mol % tetrakis(triphenylphosphine)palladium(0), to give, after desilylation, the phosphonate product 29.12.

Using the above procedures, but employing, in place of the anhydride 29.7, different anhydrides 29.1, and/or different phosphonates 29.3, the corresponding products 29.4, 29.5 and 29.6 are obtained. 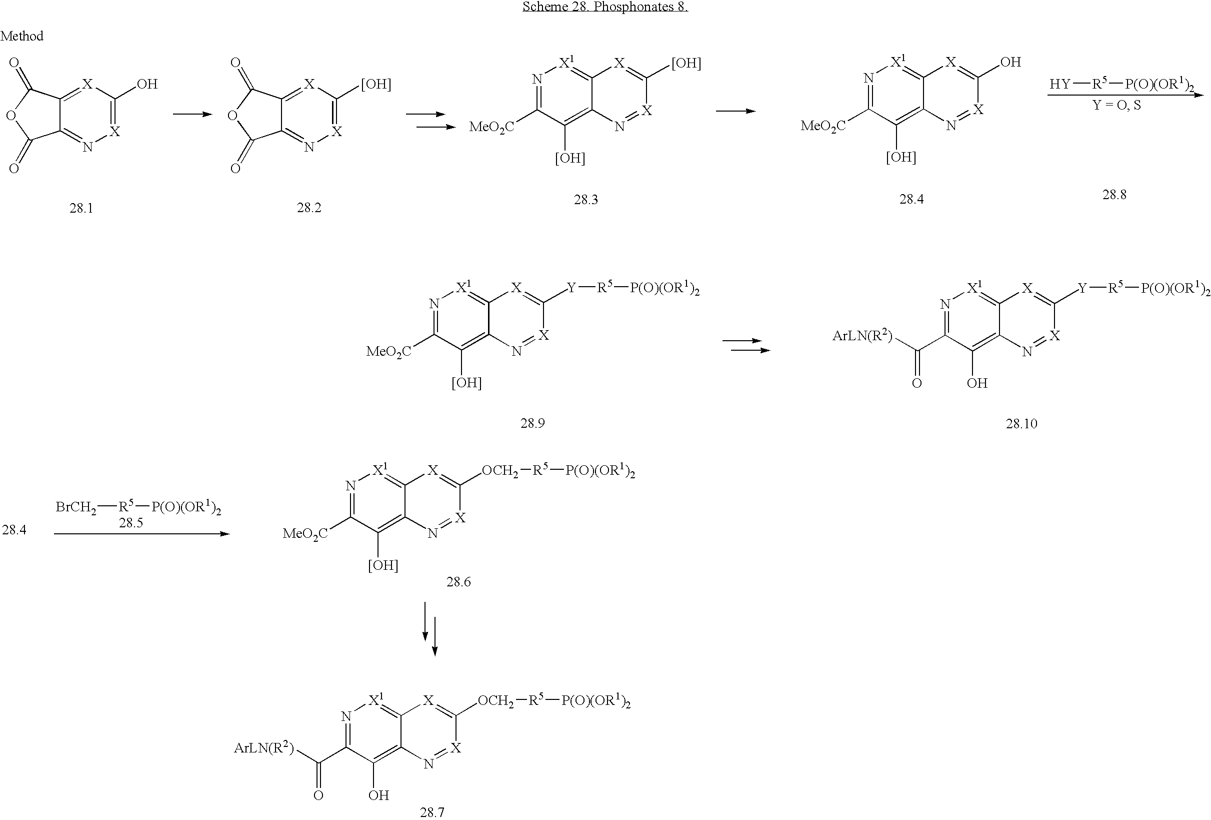 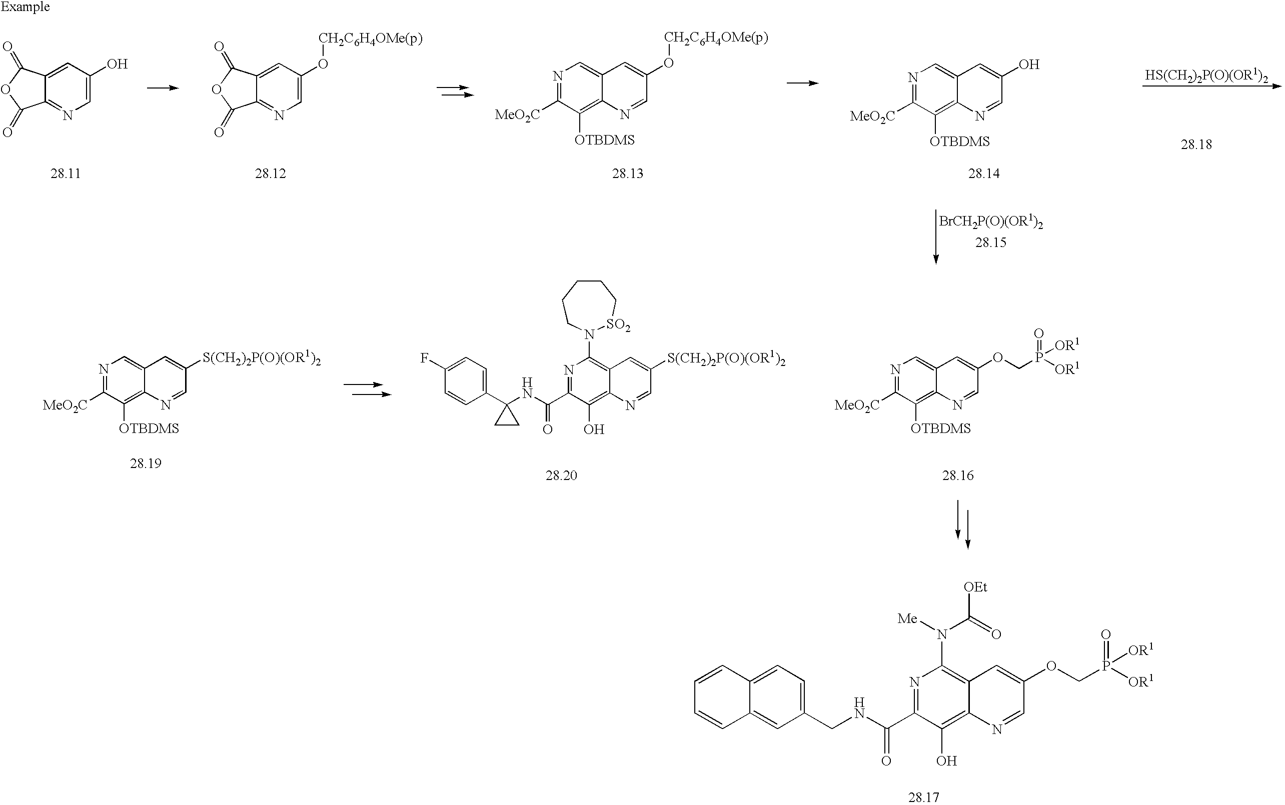 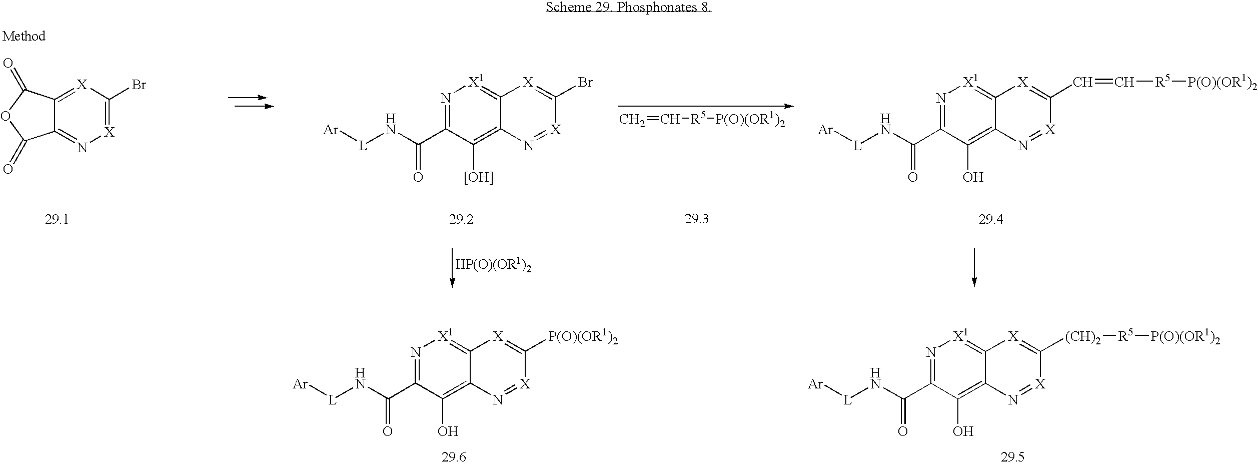 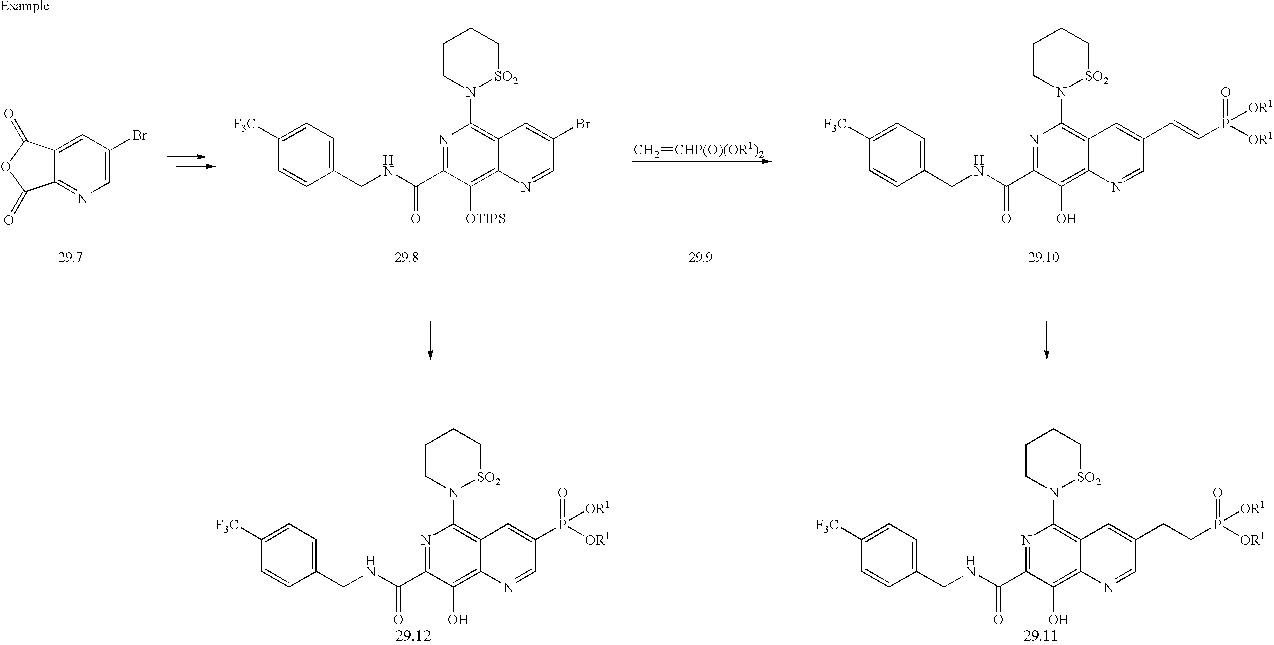
Preparation of the Intermediate Phosphonate Esters 9.

Schemes 30 and 31 illustrate methods for the preparation of the phosphonate esters 9.

Scheme 30 illustrates the preparation of phosphonate esters 9 in which the phosphonate is attached by means of a saturated or unsaturated carbon link. In this procedure, a methyl-substituted bicyclic anhydride 30.1 is converted, using the methods described above, into the amide 30.2. The product is then condensed, under basic conditions, with a dialkyl formyl-substituted phosphonate 30.3, to afford the unsaturated phosphonate 30.4. The reaction is conducted at from ambient temperature to about 100°, in a polar aprotic solvent such as dimethylformamide or dioxan, in the presence of a base such as sodium hydride, potassium tert. butoxide or lithium hexamethyldisilazide. Optionally, the product 30.4 is reduced, as described in Scheme 4, to afford the saturated analog 30.5.

For example, 2-methyl-furo[3,4-b]pyrazine-5,7-dione 30.6 (Nippon Noyaku Gakk., 1989, 14, 75) is converted, using the methods described above, into 5-(ethanesulfonyl-methyl-amino)-2-methyl-8-triisopropylsilanyloxy-pyrido[3,4-b]pyrazine-7-carboxylic acid (3,5-dichloro-benzyl)-ethyl-amide 30.7. The product is then reacted, in dimethylformamide solution at 60°, with one molar equivalent of a dialkyl formylmethyl phosphonate 30.8 (Aurora) and sodium hydride, to give, after desilylation, the unsaturated phosphonate 30.9. The product is then reacted with diimide, prepared by basic hydrolysis of diethyl azodicarboxylate, as described in Angew. Chem. Int. Ed., 4, 271, 1965, to yield the saturated product 30.10.

Using the above procedures, but employing, in place of the anhydride 30.6, different anhydrides 30.1, and/or different phosphonates 30.3, the corresponding products 30.4, and 30.5 are obtained.

Scheme 31 illustrates the preparation of phosphonate esters 9 in which the phosphonate is attached by means of an oxime linkage. In this procedure, a methyl-substituted bicyclic anhydride 31.1 is converted, using the methods described above, into the methyl-substituted amide 31.2. Benzylic bromination, as described in Scheme 25, then gives the bromomethyl analog 31.3, and oxidation, as described in Scheme 26 affords the corresponding aldehyde. The aldehyde is then converted, by reaction with hydroxylamine, into the oxime 31.5. The latter compound is then reacted, in a polar solvent such as tetrahydrofuran or dimethylformamide, in the presence of a base such as sodium hydroxide or potassium carbonate, with a dialkyl bromomethyl-substituted phosphonate 31.6, to prepare, after deprotection of the phenolic hydroxyl group, the oxime derivative 31.7.

For example, 2-methyl-furo[3,4-b]pyrazine-5,7-dione 30.6 (Nippon Noyaku Gakk., 1989, 14, 75) is converted, using the methods described above, into 5-(ethenesulfonyl-methyl-amino)-2-formyl-8-triisopropylsilanyloxy-pyrido[3,4-b]pyrazine-7-carboxylic acid 4-fluoro-benzylamide 31.9. The aldehyde is then reacted in tetrahydrofuran solution with three molar equivalents of hydroxylamine hydrochloride and sodium acetate, to produce the oxime 31.10. The latter compound is then reacted in dioxan solution at ambient temperature, with one molar equivalent of a dialkyl bromopropyl phosphonate 31.11 (Synthelec) and potassium carbonate, to yield, after desilylation of the phenolic hydroxyl group, the oxime ether 31.12.

Using the above procedures, but employing, in place of the anhydride 31.8, different anhydrides 31.1, and/or different phosphonates 31.6, the corresponding products 31.7 are obtained. 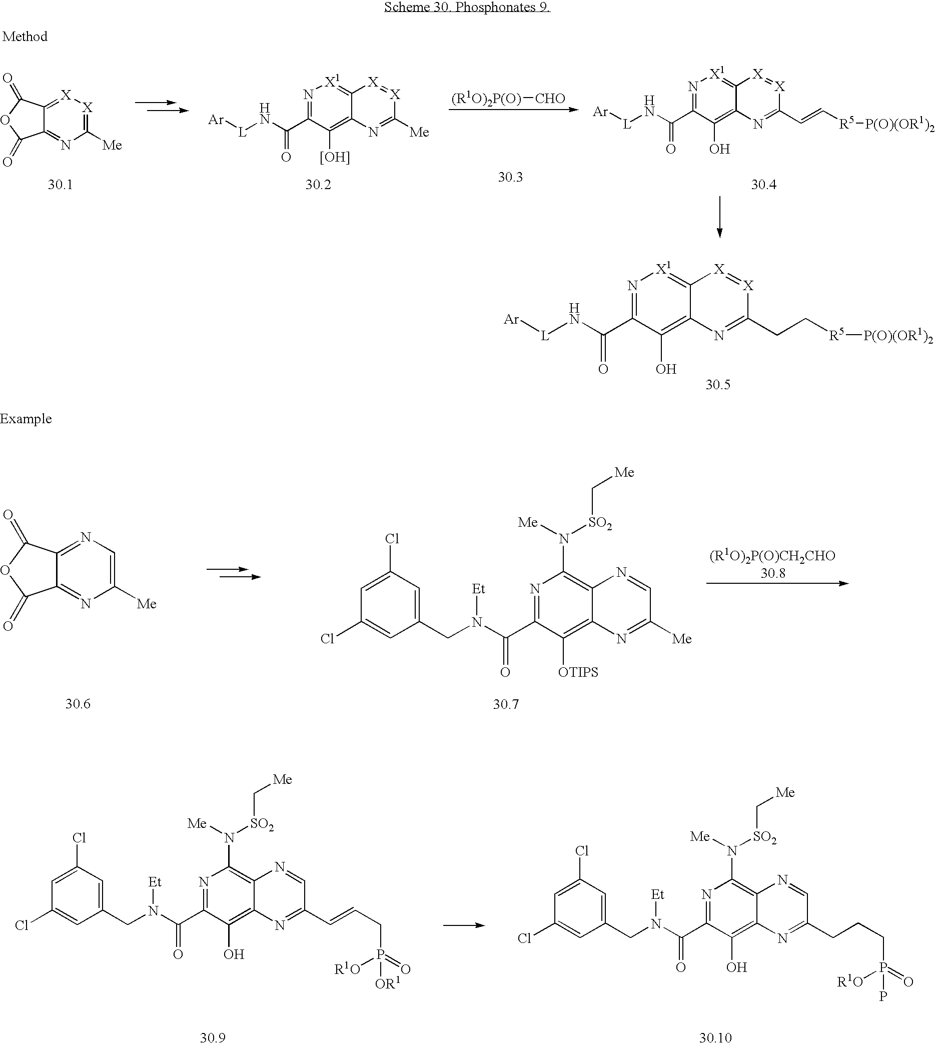 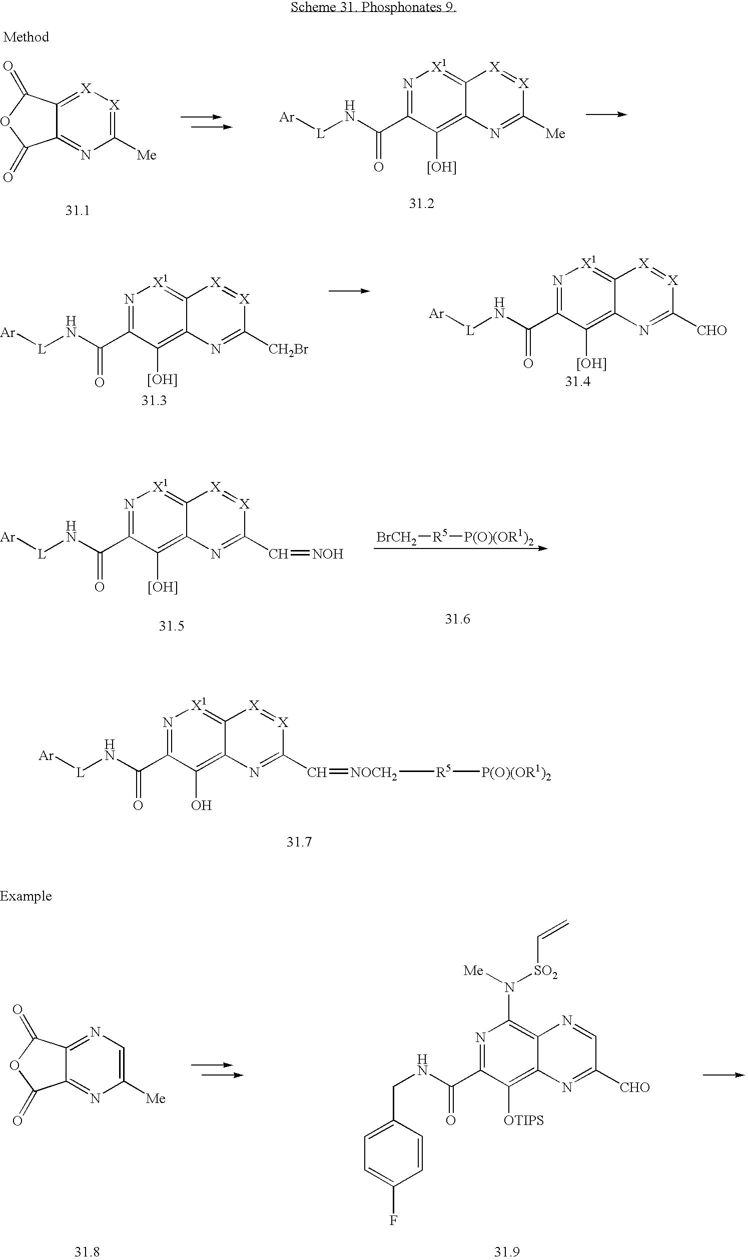 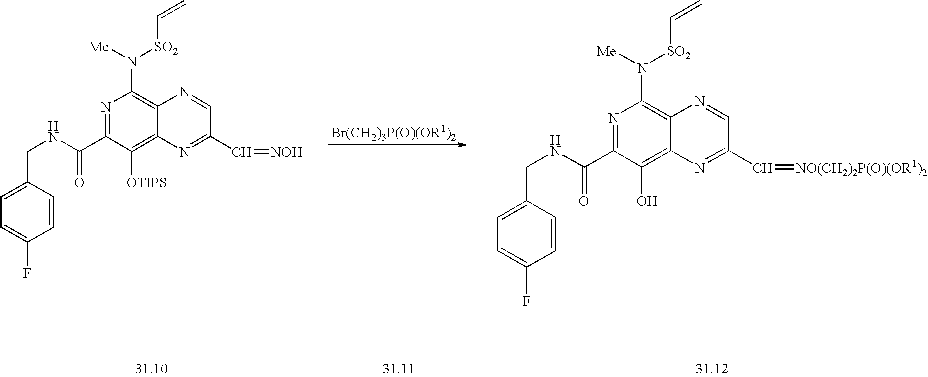
Synthesis of Formula IV Pyrimidine and V Pyrimidinone Phosphonate Compounds

Dihydroxypyrimidine carboxamide (WO 03/035076A1) and N-substituted hydroxypyrimidinone carboxamide (WO 03/035077A1) compounds have been disclosed.

Preparation of Formula IVa-d and Formula Va-d Phosphonate Esters.

Structures of exemplary pyrimidine Formula IV phosphonate esters IVa-d are shown in Chart 1. Structures of exemplary pyrimidine Formula II phosphonate esters Va-d are shown in Chart 2. Ring substituents R¹, R^{2a}, R^{2b}, R³, R⁴, and R⁵ are as previously defined. Phosphonate ester substituent R^{x} is as previously defined. Compounds of Formula IVa-d and Formula Va-d may each be an active pharmaceutical ingredient, or an intermediate for preparing other compounds of the invention by subsequent chemical modifications.

Compounds of Formula IVa-d and Formula Va-d incorporate a phosphonate group (R¹O)₂P(O) connected to the pyrimidine and pyrimidinone scaffold, respectively, by means of a divalent and variable linking group, designated as “L” in the attached structures. Charts 3 and 4 illustrates examples of the phosphonate linking groups (L-A³) present in the structures IVa-d and Va-d.

The methods described for the introduction of phosphonate substituents are, with modifications made by one skilled in the art, transferable within the phosphonate esters IVa-d and Va-d. For example, reaction sequences which produce the phosphonates IVa are, with appropriate modifications, applicable to the preparation of the phosphonates IVb-d and Va-d. Methods described below for the attachment of phosphonate groups by means of reactive substituents such as OH, Br, NH₂, CH₃, CH₂Br, COOH, CHO etc are applicable to each of the scaffolds IVa-d and Va-d.

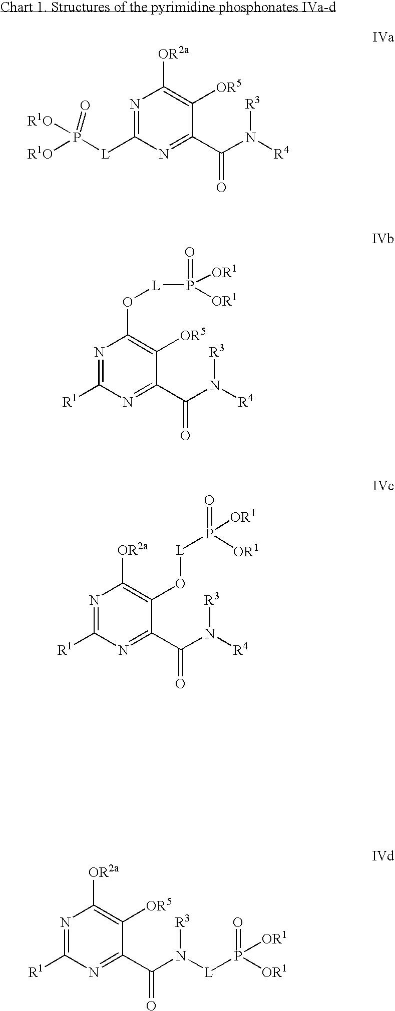

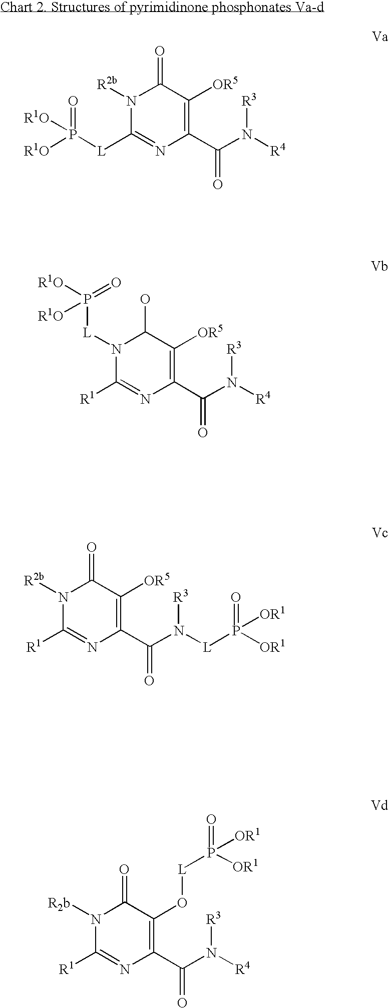

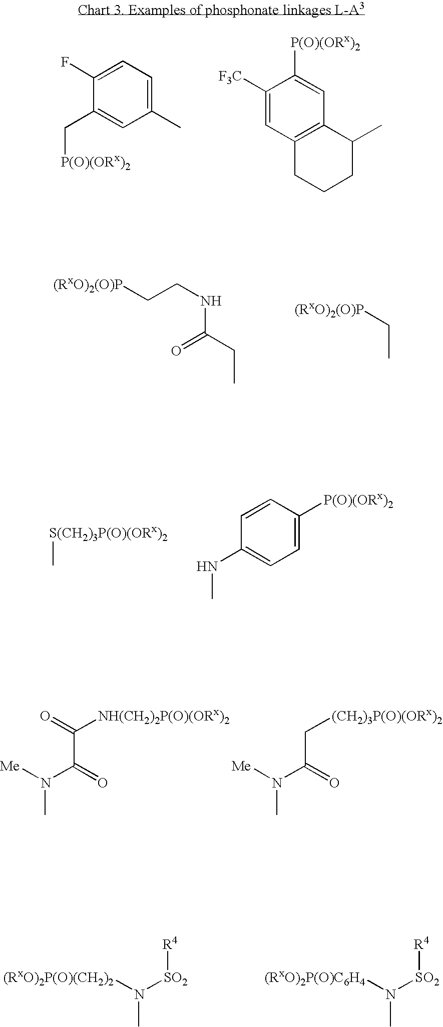

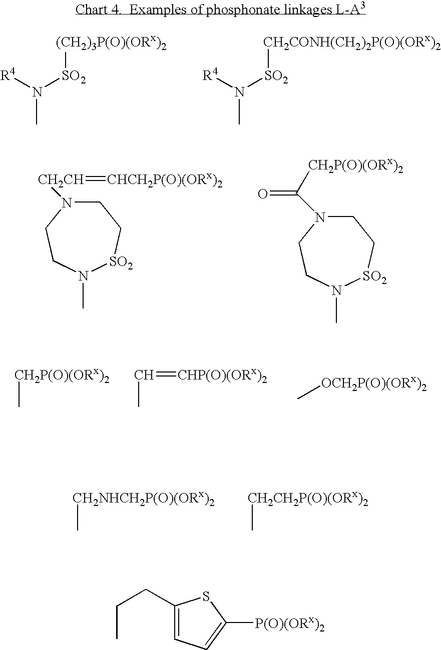

Schemes 1-31 illustrate the syntheses of the phosphonate compounds of this invention, Formulas I and II, and of the intermediate compounds necessary for their synthesis.

Scheme 32 illustrates methods for the interconversion of phosphonate diesters, monoesters and acids, and Scheme 33 illustrates methods for the preparation of carbamates. Schemes 34-37 illustrate the conversion of phosphonate esters and phosphonic acids into carboalkoxy-substituted phosphonbisamidates, phosphonamidates, phosphonate monoesters, phosphonate diesters. Scheme 38 illustrates further synthesis of gem-dialkyl amino phosphonate reagents for preparation of Formulas I and II compounds.

Protection of Reactive Substituents.

Depending on the reaction conditions employed, it may be necessary to protect certain reactive substituents from unwanted reactions by protection before the sequence described, and to deprotect the substituents afterwards, according to the knowledge of one skilled in the art. Protection and deprotection of functional groups are described, for example, in Protective Groups in Organic Synthesis, by T. W. Greene and P. G. M. Wuts, Wiley, Second Edition 1990. Reactive substituents which may be protected are shown in the accompanying schemes as, for example, [OH], [SH], [NH] etc. Protecting groups are also exemplified as “PG”. The selection of a suitable stage in the synthetic sequence for the introduction of the phosphonate group is made by one skilled in the art, depending on the reactivity and stability of the substrates in a given reaction sequence.

Protection of Phosphonate Esters

Scheme 3a depicts the preparation of phosphonate esters IVd and Vd in which the phosphonate group is directly attached to the group Ar. In this procedure, a bromo-substituted amine 3.1, in which Ar is an aromatic or heteroaromatic group, is reacted, in the presence of a palladium catalyst, with a dialkyl phosphite 3.2 to yield the aryl phosphonate 3.3. The preparation of arylphosphonates by means of a coupling reaction between aryl bromides and dialkyl phosphites is described in J. Med. Chem., 35, 1371, 1992. This reaction is performed in an inert solvent such as toluene, in the presence of a base such as triethylamine and a palladium (0) catalyst such as tetrakis(triphenylphosphine)palladium(0). Optionally, the amine group is protected prior to the coupling reaction, and deprotected afterwards.

Amine reagent 3.3 is reacted with the ester 3.4 to afford the amide 3.5, and with the ester 3.6 to afford the amide 3.7. The conversion of esters into amides is described in Comprehensive Organic Transformations, by R. C. Larock, VCH, 1989, p. 987. The reactants are combined in a solvent such as toluene or xylene, in the presence of a base such as sodium methoxide under azeotropic conditions, or of a dialkyl aluminum or trialkyl tin derivative of the amine. The use of trimethylaluminum in the conversion of esters to amides is described in J. Med. Chem. Chim. Ther., 34, 1999, 1995, and Syn. Comm., 25, 1401, 1995. The reaction is conducted in an inert solvent such as dichloromethane or toluene. The conversion of esters such as 3.4 and 3.6, or the corresponding carboxylic acids, into amides is described in WO 03035077 A1, Optionally, the 5-hydroxyl group of the ester 3.4 and 3.6 is protected, for example as a p-toluenesulfonyl derivative, prior to reaction with the amine component 3.3.

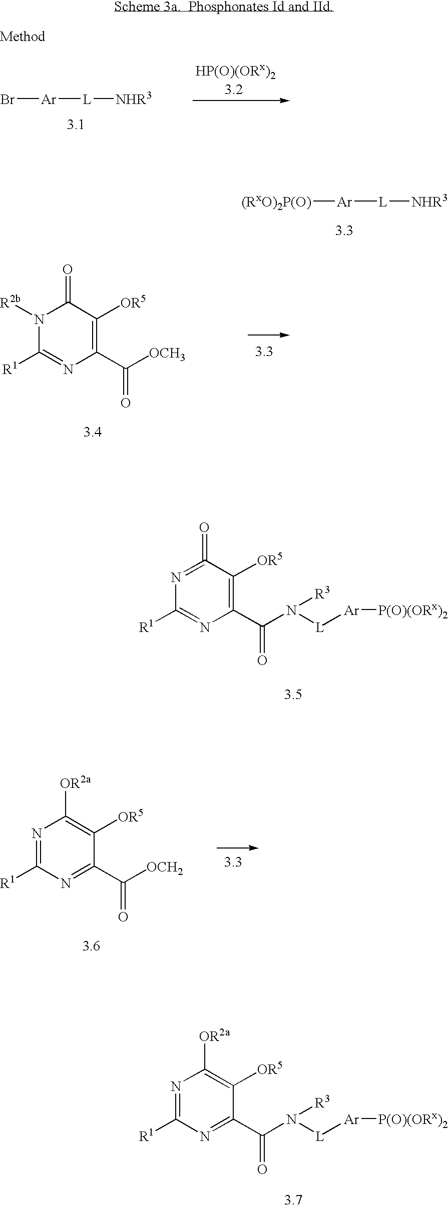

For example, 3-bromo-4-fluorobenzylamine 3.8 (Lancaster) is reacted in toluene solution at ca. 100° C., with one molar equivalent of a dialkyl phosphite 3.9, triethylamine and 3 mol % of tetrakis(triphenylphosphine)palladium(0), to give the phosphonate product 3.10 in Scheme 3b. Compound 3.10 is then reacted, in toluene solution at reflux temperature with 3.11 to yield the pyrimidine amide 3.12. Alternatively, 3.10 is reacted, in toluene solution at reflux temperature with 3.13 to yield the pyrimidinone amide 3.14

Using the above procedures, but employing, in place of the amine 3.8, different amines 3.1, and/or different esters 3.4, the corresponding amides 3.5 are obtained.

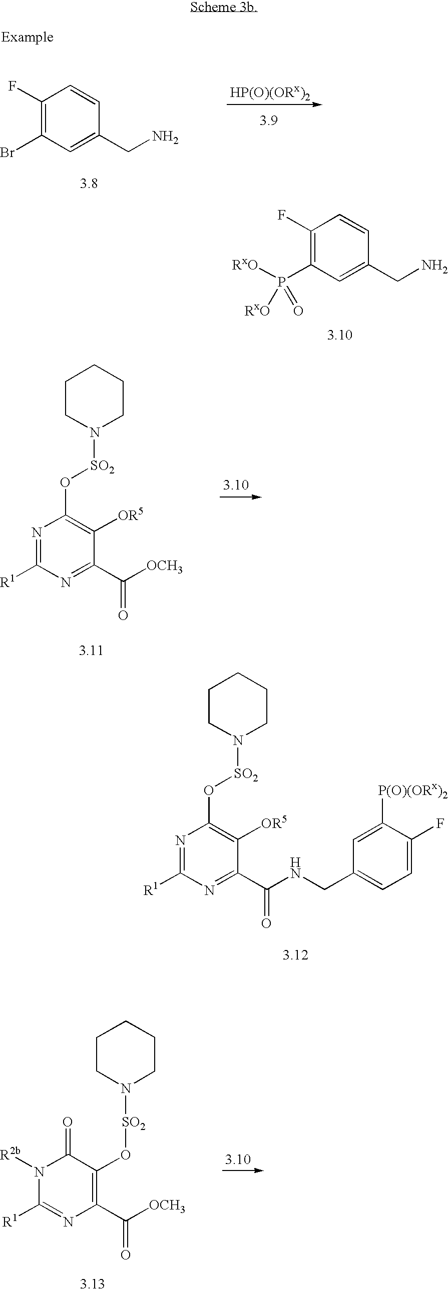

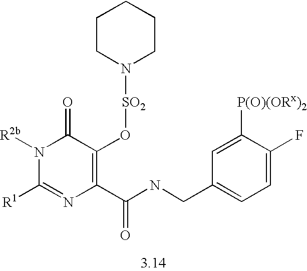

Scheme 4 depicts the preparation of phosphonate esters 1 in which the phosphonate group is attached by means of a saturated or unsaturated alkylene chain. In this procedure, a bromo-substituted amine 4.1, in which Ar is an aryl or heterocycle group, is subjected to a Heck coupling reaction, in the presence of a palladium catalyst, with a dialkyl alkenyl phosphonate 4.2, in which R^{5a} is a direct bond, a divalent group such as alkylene, alkenylene, alkynylene or cycloalkylene group, optionally incorporating a heteroatom O, S or N, ethyleneoxy, polyethyleneoxy, or a functional group such as an amide, ester, oxime, sulfoxide or sulfone etc, or an optionally substituted aryl, heterocycle or aralkyl group, to give the amine 4.3. The coupling of aryl halides with olefins by means of the Heck reaction is described, for example, in Advanced Organic Chemistry, by F. A. Carey and R. J. Sundberg, Plenum, 2001, p. 503ff and in Acc. Chem. Res., 12, 146, 1979. The aryl bromide and the olefin are coupled in a polar solvent such as dimethylformamide or dioxane, in the presence of a palladium(0) catalyst such as tetrakis(triphenylphosphine)palladium(0) or a palladium(II) catalyst such as palladium(II) acetate, and optionally in the presence of a base such as triethylamine or potassium carbonate. Optionally, the amine substituent is protected prior to the coupling reaction, and deprotected afterwards. The phosphonate amine 4.3 is then coupled, as described above, with the ester 4.4, or the corresponding carboxylic acid, to produce the amide 4.5. Optionally, the double bond is reduced to give the saturated analog 4.6. The reduction of olefinic bonds is described in Comprehensive Organic Transformations, by R. C. Larock, VCH, 1989, p. 6ff. The transformation is effected by means of catalytic hydrogenation, for example using a palladium on carbon catalyst and hydrogen or a hydrogen donor, or by the use of diimide or diborane.

For example, 3-bromo-4-methoxybenzylamine 4.7 (Lancaster) is reacted in dioxane solution with one molar equivalent of a dialkyl vinyl phosphonate 4.8 (Aldrich) and potassium carbonate, to yield the olefinic phosphonate 4.9. The product is then reacted, as described above, with 6-methyl ester 4.10, prepared as described in Scheme 1A, to give the amide 4.11. The latter compound is reacted with diimide, prepared by basic hydrolysis of diethyl azodicarboxylate, as described in Angew. Chem. Int. Ed., 4, 271, (1965), to yield the saturated product 4.12.

Using the above procedures, but employing, in place of the amine 4.7, different amines 4.1, and/or different phosphonates 4.2, and/or different bicyclic esters 4.4, the corresponding amides 4.5 and 4.6 are obtained.

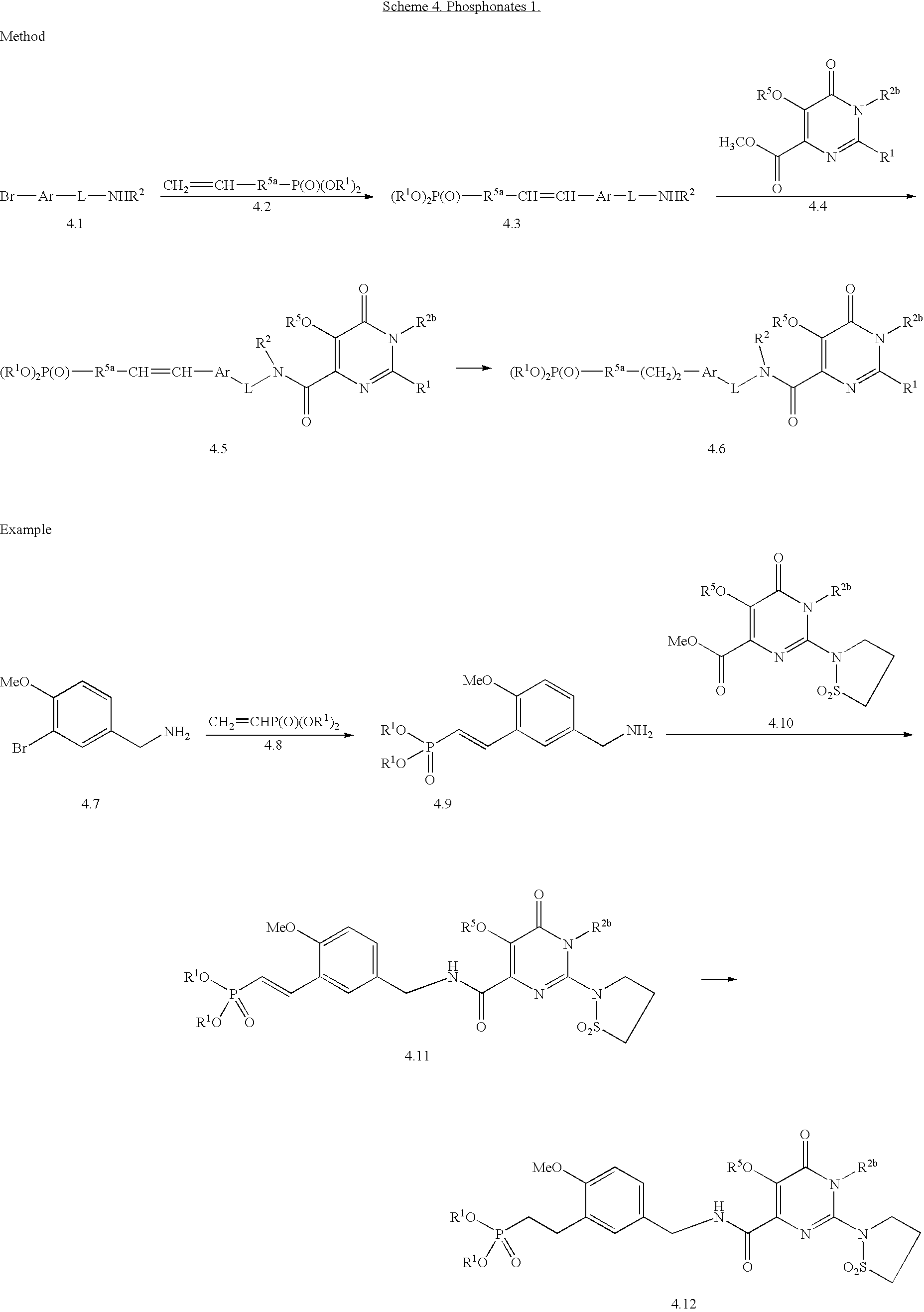

Scheme 5 depicts the preparation of phosphonate esters IVd in which the phosphonate group is attached by means of an amide linkage. In this procedure, the amine group of a carboxy-substituted amine 5.1 is protected to afford the derivative 5.2. The protection of amino groups is described in Protective Groups in Organic Synthesis, by T. W. Greene and P. G. M. Wuts, Wiley, Second Edition 1990, p. 309ff. Amino groups are protected, for example by alkylation, such as by mono or dibenzylation, or by acylation. The conversion of amines into mono or dibenzylamines, for example by treatment with benzyl bromide in a polar solvent such as acetonitrile or aqueous ethanol, in the presence of a base such as triethylamine or sodium carbonate, is described in Protective Groups in Organic Synthesis, by T. W. Greene and P. G. M. Wuts, Wiley, Second Edition 1990, p. 364. The N-protected carboxylic acid 5.2 is then coupled with an amino-substituted dialkyl phosphonate 5.3, in which the group R^{5a} is as defined in Scheme 4, to yield the amide 5.4. The preparation of amides from carboxylic acids and derivatives is described, for example, in Organic Functional Group Preparations, by S. R. Sandler and W. Karo, Academic Press, 1968, p. 274, and in Comprehensive Organic Transformations, by R. C. Larock, VCH, 1989, p. 972ff. The carboxylic acid is reacted with the amine in the presence of an activating agent, such as, for example, dicyclohexylcarbodiimide or diisopropylcarbodiimide, optionally in the presence of, for example, hydroxybenzotriazole, N-hydroxysuccinimide or N-hydroxypyridone, in a non-protic solvent such as, for example, pyridine, DMF or dichloromethane, to afford the amide.

Alternatively, the carboxylic acid is first converted into an activated derivative such as the acid chloride, anhydride, mixed anhydride, imidazolide and the like, and then reacted with the amine, in the presence of an organic base such as, for example, pyridine, to afford the amide.

The conversion of a carboxylic acid into the corresponding acid chloride is effected by treatment of the carboxylic acid with a reagent such as, for example, thionyl chloride or oxalyl chloride in an inert organic solvent such as dichloromethane, optionally in the presence of a catalytic amount of dimethylformamide.

The amino-protecting group is then removed from the product 5.4 to give the free amine 5.5. Deprotection of amines is described in Protective Groups in Organic Synthesis, by T. W. Greene and P. G. M. Wuts, Wiley, Second Edition 1990, p. 309ff. The amine is then coupled with the carboxylic acid 5.6, as described above, to produce the amide 5.7.

For example, 4-carboxycyclohexylmethylamine 5.8 (Aldrich) is converted into the phthalimido derivative 5.9 (pht=phthalimide). The conversion of amines into phthalimido derivatives is described in Protective Groups in Organic Synthesis, by T. W. Greene and P. G. M. Wuts, Wiley, Second Edition 1990, p. 358. The conversion is effected by reaction of the amine with an equimolar amount of 2-carbomethoxybenzoyl chloride, N-carboethoxyphthalimide, or preferably, phthalic anhydride. The reaction is performed in an inert solvent such as toluene, dichloromethane or acetonitrile, to prepare the phthalimido derivative 5.9. This material is then reacted with one molar equivalent of a dialkyl aminoethyl phosphonate 5.10, (J. Org. Chem., (2000), 65, 676) and dicyclohexylcarbodiimide in dimethylformamide, to give the amide 5.11. The phthalimido protecting group is then removed, for example by reaction with ethanolic hydrazine at ambient temperature, as described in J. Org. Chem., 43, 2320, (1978), to afford the amine 5.12. This compound is coupled in dimethylformamide solution with 6-carboxylic acid 5.13, to afford the amide 5.14.

Using the above procedures, but employing, in place of the amine 5.8, different amines 5.1, and/or different phosphonates 5.3, and/or different carboxylic acids 5.6, the corresponding products 5.7 are obtained.

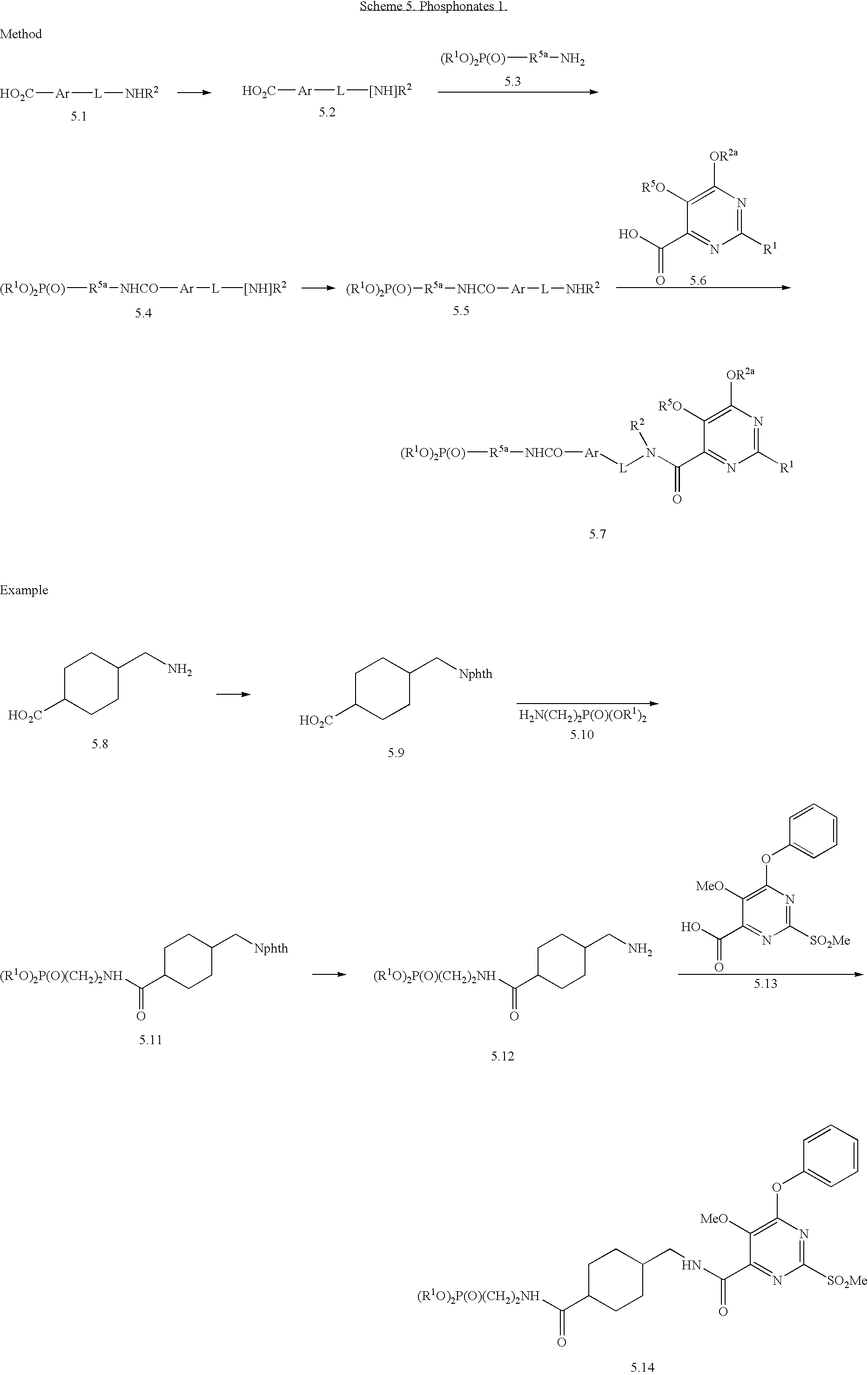

Scheme 6 depicts the preparation of phosphonates Vd in which the phosphonate is attached by means of an ether linkage. In this procedure, the amino group of a hydroxy-substituted amine 6.1 may be protected (PG=protecting group), as described above, to give the derivative 6.2. The alcohol is then reacted, with base catalysis, with a dialkyl bromomethyl phosphonate 6.3, in which the group R⁵ is as defined in Scheme 4. The reaction is conducted in a polar aprotic solvent such as tetrahydrofuran, dimethylformamide or dimethylsulfoxide, in the presence of a base such as potassium carbonate, for cases in which Ar is an aromatic group, or a strong base such as sodium hydride, for cases in which Ar is an aliphatic group. The amino group of the resulting ether 6.4 is then deprotected, as previously described, to give the amine 6.5. The amine is then reacted with the ester 6.6, as described in Scheme 3, to give the amide 6.7.

For example, N-methyl 3-hydroxyphenethylamine 6.8 is reacted with one molar equivalent of acetyl chloride in dichloromethane containing pyridine, to give the N-acetyl product 6.9. The product is then reacted at ca. 60° C. in dimethylformamide (DMF) solution with one molar equivalent of a dialkyl 3-bromopropenyl phosphonate 6.10 (Aurora) and cesium carbonate, to produce the ether 6.11. The N-acetyl group is then removed, for example by treatment with hog kidney acylase, as described in Tetrahedron, 44, 5375, (1988), to give the amine 6.12. The product is then reacted in toluene solution at reflux, 6.13, to yield the amide 6.14.

Using the above procedures, but employing, in place of the amine 6.8, different amines 6.1, and/or different phosphonates 6.3, and/or different bicyclic esters 6.6, the corresponding products 6.7 are obtained.

Scheme 7 depicts the preparation of phosphonates Vd in which the phosphonate is attached by means of an ether or thioether linkage. In this procedure, a N-protected hydroxyamine 6.2, in which Ar is an aromatic moiety, is subjected to a Mitsunobu reaction with a hydroxy or mercapto-substituted dialkyl phosphonate 7.1, in which R^{5a} is as defined in Scheme 4, to prepare the ether or thioether product 7.2. The preparation of aromatic ethers and thioethers by means of the Mitsunobu reaction is described, for example, in Comprehensive Organic Transformations, by R. C. Larock, VCH, 1989, p. 448, and in Advanced Organic Chemistry, Part B, by F. A. Carey and R. J. Sundberg, Plenum, 2001, p. 153-4 and in Org. React., 1992, 42, 335. The phenol and the alcohol or thiol component are reacted together in an aprotic solvent such as, for example, tetrahydrofuran or dioxane, in the presence of a dialkyl azodicarboxylate and a triarylphosphine, to afford the ether or thioether products. The N-protecting group is then removed and the resultant amine is converted, as described in Scheme 6, into the amide 7.3.

For example, N-acetyl 3,5-dichloro-4-hydroxybenzylamine 7.4 is reacted in a tetrahydrofuran solution with one molar equivalent of a dialkyl mercaptoethyl phosphonate 7.5, (Zh. Obschei. Khim., 1973, 43, 2364) diethyl azodicarboxylate and tri-o-tolylphosphine, to afford the thioether product 7.6. The N-acetyl group is removed, as described in Scheme 6, and the amine 7.7 is then reacted with methyl ester 7.8 (TBDMS=tert-butyldimethylsilyl), to afford the amide 7.9.

Using the above procedures, but employing, in place of the amine 7.4, different amines 6.2, and/or different phosphonates 7.2, the corresponding products 7.3 are obtained.

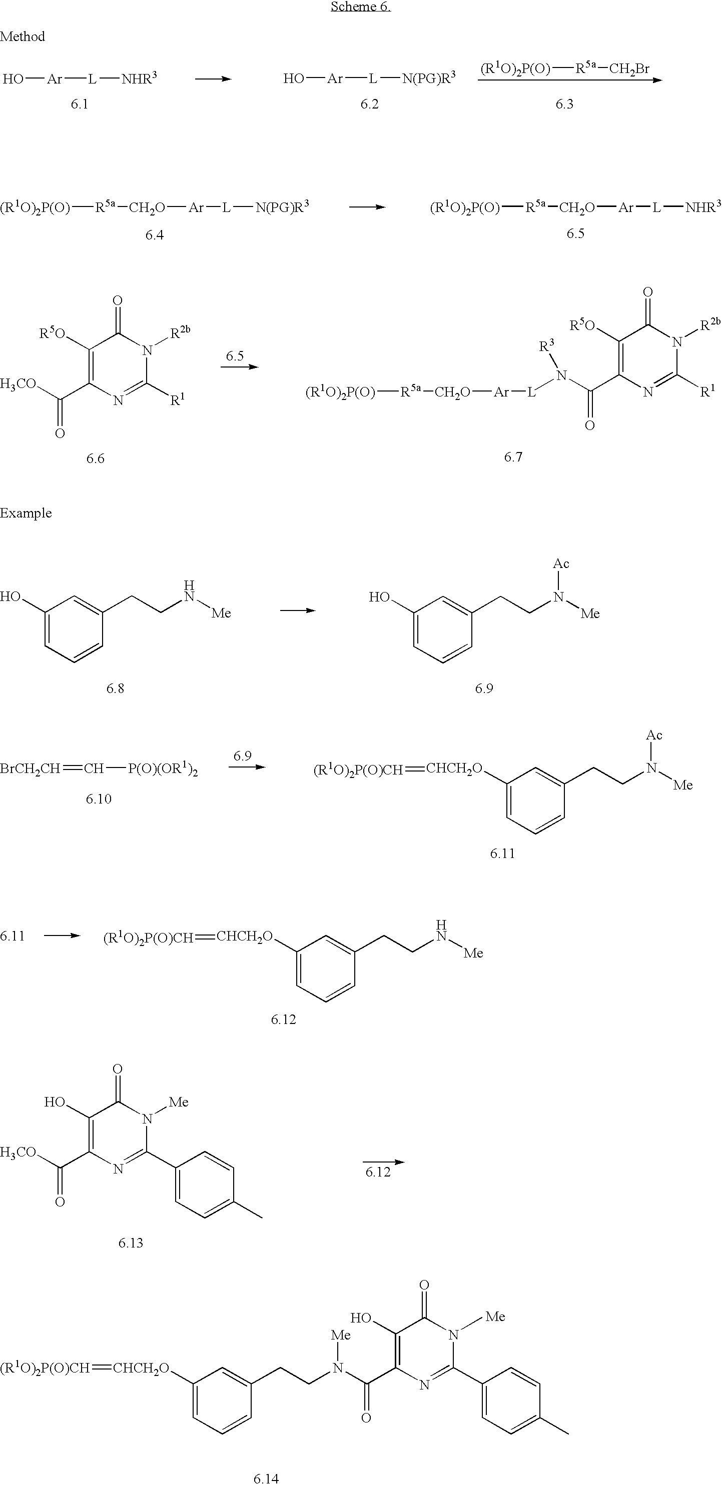

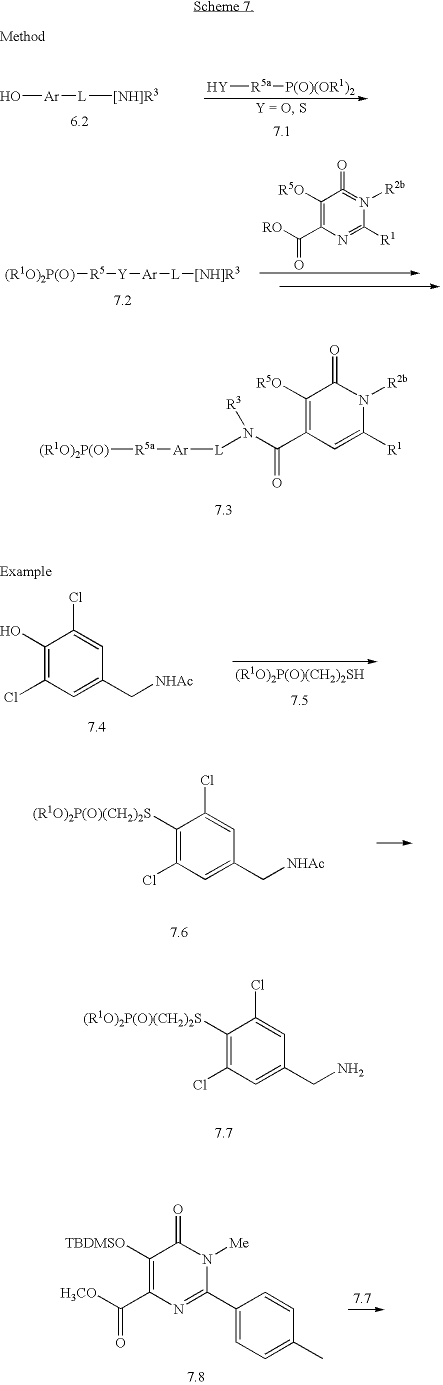

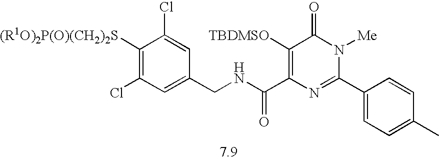

Scheme 8 depicts the preparation of phosphonates IVd in which the phosphonate is attached by means of an alkylene chain incorporating an amide linkage. In this procedure, an amine 8.1 is reacted with a bromoalkyl ester 8.2, in which R^{5a} is as defined in Scheme 4, to yield the alkylated amine 8.3. The preparation of substituted amines by the reaction of amines with alkyl halides is described, for example, in Comprehensive Organic Transformations, by R. C. Larock, VCH, 1989, p. 397. Equimolar amounts of the reactants are combined in a polar solvent such as an alkanol or dimethylformamide and the like, in the presence of a base such as cesium carbonate, diazabicyclononene or dimethylaminopyridine, to yield the substituted amine. The ester group is then hydrolyzed to give the carboxylic acid 8.4, and this compound is then coupled, as described in Scheme 5, with a dialkyl aminoalkyl phosphonate 8.5, to produce the aminoamide 8.6. Optionally, the amino group of the amine 8.4 is protected prior to the coupling reaction, and deprotected afterwards. The product is then reacted with the bicyclic hydroxyester 8.7 to afford the amide 8.8.

For example, 4-trifluoromethylbenzylamine 8.9 is reacted in dimethylformamide with one molar equivalent of methyl bromoacetate 8.10 and potassium carbonate to give the ester 8.11. Hydrolysis, employing one molar equivalent of lithium hydroxide in aqueous dimethoxyethane, affords the carboxylic acid 8.12, and this compound is coupled in tetrahydrofuran solution with a dialkyl aminomethyl phosphonate 8.13 (Aurora), in the presence of dicyclohexylcarbodiimide, to give the aminoamide 8.14. The product is then reacted with 4-sulfonamide, 6-methyl ester 8.15, prepared by the methods described above, to yield the amide 8.16.

Using the above procedures, but employing, in place of the amine 8.9, different amines 8.1, and/or different bromoesters 8.2, and/or different phosphonates 8.5, and/or different hydroxyesters 8.7, the corresponding products 8.8 are obtained.

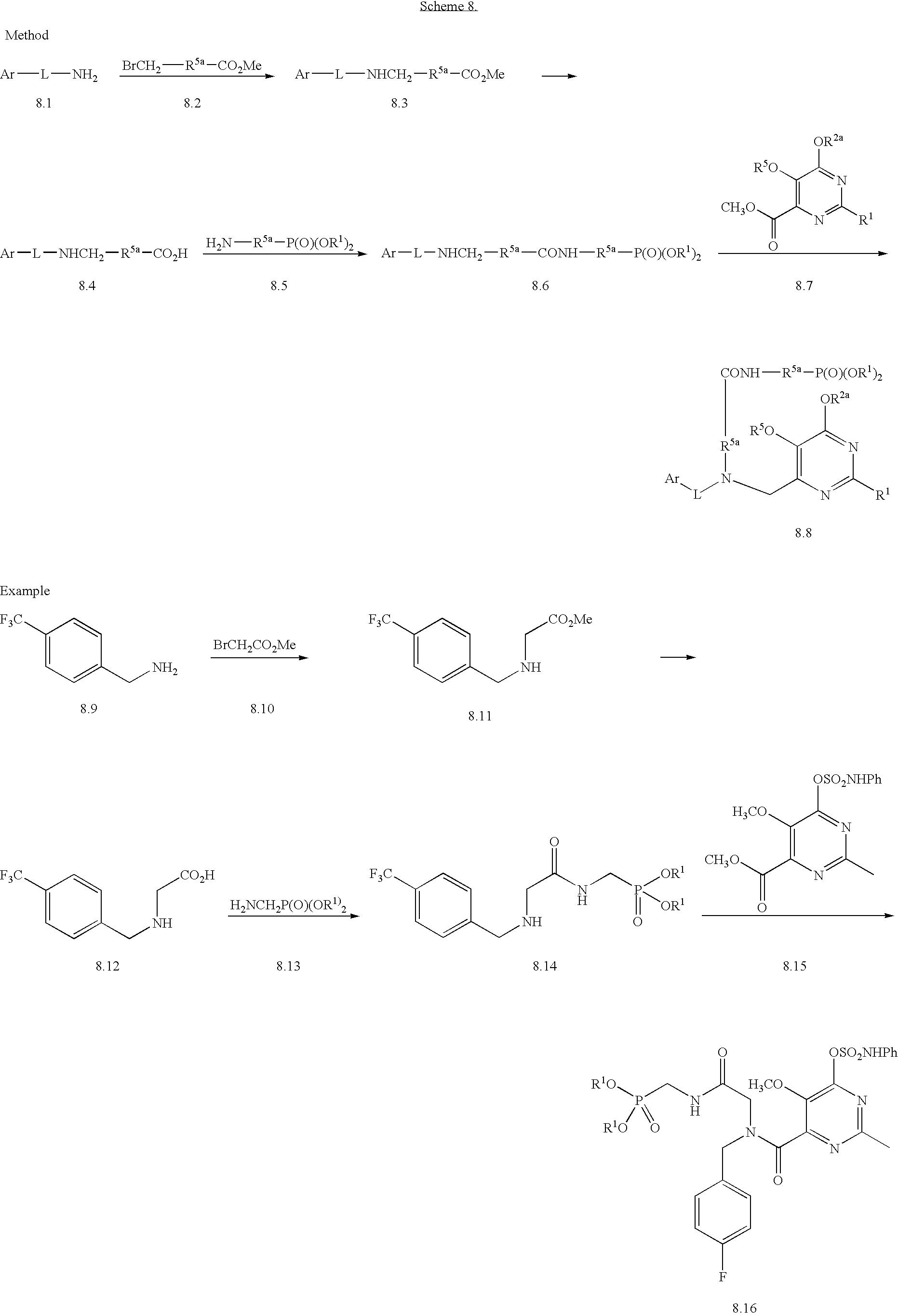

Scheme 9 depicts the preparation of phosphonates Vd in which the phosphonate is attached by means of a variable carbon chain. In this procedure, a primary amine 9.1 is subjected to a reductive amination reaction with a dialkyl formyl-substituted phosphonate 9.2, in which R⁵ is as defined in Scheme 4, to afford the alkylated amine 9.3. The preparation of amines by means of reductive amination procedures is described, for example, in Comprehensive Organic Transformations, by R. C. Larock, VCH, p. 421, and in Advanced Organic Chemistry, Part B, by F. A. Carey and R. J. Sundberg, Plenum, 2001, p. 269. In this procedure, the amine component and the aldehyde or ketone component are reacted together in a polar solvent in the presence of a reducing agent such as, for example, borane, sodium cyanoborohydride, sodium triacetoxyborohydride or diisobutylaluminum hydride, optionally in the presence of a Lewis acid, such as titanium tetraisopropoxide, as described in J. Org. Chem., 55, 2552, 1990. The product 9.3 is then reacted, as described previously, with the bicyclic ester 9.4 to give the amide 9.5.

For example, 3,4-dichlorobenzylamine is reacted in methanol solution with one molar equivalent of a dialkyl 3-formylphenyl phosphonate 9.7, (Epsilon) and sodium cyanoborohydride, to yield the alkylated product 9.8. This compound is then reacted with 2-dimethylcarbamoyl-5,6-dihydroxy-pyrimidine-4-carboxylic acid methyl ester 9.9, prepared using the methods described above, from the corresponding bromo compound and N-methyl methanesulfonamide, to give the amide 9.10.

Using the above procedures, but employing, in place of the amine 9.6, different amines 9.1, and/or different phosphonates 9.2, and/or different bicyclic esters 9.4, the corresponding products 9.5 are obtained.

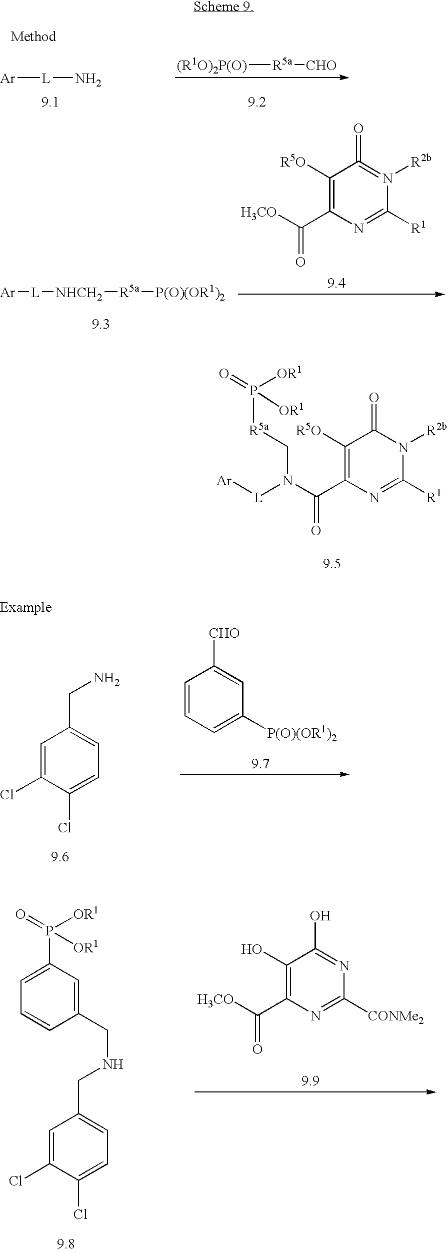

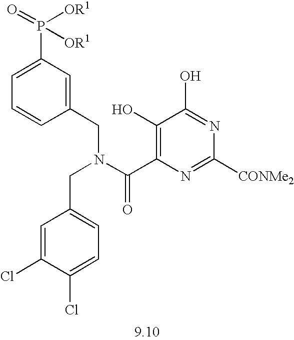

Scheme 10 depicts an alternative method for the preparation of phosphonates Vd in which the phosphonate is attached by means of a variable carbon chain. In this procedure, the phenolic group of a bicyclic amide 10.1, prepared as described above, and in WO 02 30930 A2, is protected to give the product 10.2. The protection of phenolic hydroxyl groups is described in Protective Groups in Organic Synthesis, by T. W. Greene and P. G. M. Wuts, Wiley, Second Edition 1990, p. 10ff. For example, hydroxyl substituents are protected as trialkylsilyloxy ethers. Trialkylsilyl groups are introduced by the reaction of the phenol with a chlorotrialkylsilane and a base such as imidazole, for example as described in Protective Groups in Organic Synthesis, by T. W. Greene and P. G. M. Wuts, Wiley, Second Edition 1990, p. 10, p. 68-86. Alternatively, phenolic hydroxyl groups are protected as benzyl or substituted benzyl ethers, or as acetal ethers such as methoxymethyl or tetrahydropyranyl. The O-protected amide 10.2 is then reacted with the phosphonate-substituted trifluoromethanesulfonate 10.3, in which R^{5a} is as defined in Scheme 4, to produce the alkylated amide 10.4. The alkylation reaction is conducted between equimolar amounts of the reactants in an aprotic organic solvent such as dimethylformamide or dioxane, in the presence of a strong base such as lithium hexamethyl disilylazide or sodium hydride, at from ambient temperature to about 90° C. The hydroxyl group is then deprotected to give the phenol 10.5. Deprotection of phenolic hydroxyl groups is described in Protective Groups in Organic Synthesis, by T. W. Greene and P. G. M. Wuts, Wiley, Second Edition 1990, p. 10ff. For example, silyl protecting groups are removed by reaction with tetrabutylammonium fluoride, benzyl groups are removed by catalytic hydrogenation and acetal ethers are removed by treatment with acids.

Amide 10.7 is reacted with one molar equivalent of tert-butyl chlorodimethylsilane and imidazole in dichloromethane, to give 5-(tert-butyl-dimethyl-silanyloxy)-1-methyl-6-oxo-2-phenyl-1,6-dihydro-pyrimidine-4-carboxylic acid (naphthalen-2-ylmethyl)-amide 10.8. This compound 10.8 is then reacted at ambient temperature in dioxane solution with one molar equivalent of sodium hydride, followed by the addition of a dialkyl trifluoromethanesulfonyloxymethyl phosphonate 10.9 (Tet. Lett., 1986, 27, 1477), to afford the alkylated product 10.10. Deprotection, by reaction with tetrabutylammonium fluoride in tetrahydrofuran, then yields the product 10.11.

Using the above procedures, but employing, in place of the amide 10.7, different amides 10.1, and/or different phosphonates 10.3, the corresponding products 10.5 are obtained.

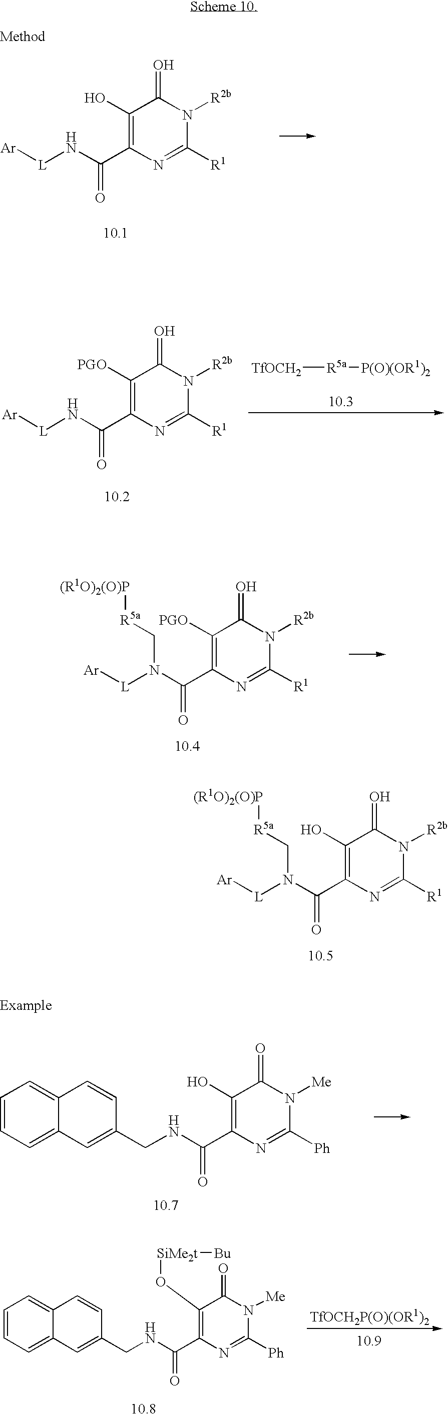

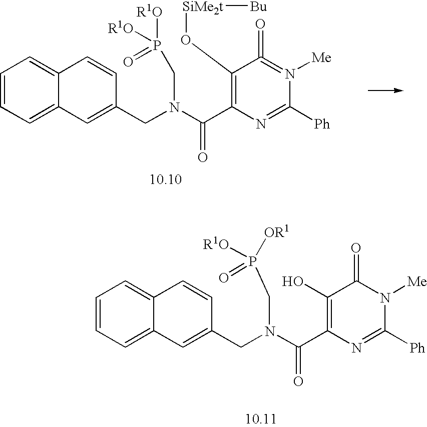

Schemes 11-15 illustrate methods for the preparation of the 2-phosphonate esters IVa and Va.

Scheme 11 depicts the preparation of 2-substituted pyrimidyl phosphonates Va in which the phosphonate is attached by means of a heteroatom O, S or N, and a variable carbon chain. In this procedure, an amide 11.1, prepared as previously described, is reacted in an aprotic solvent such as dichloromethane, hexachloroethane or ethyl acetate with a free radical brominating agent such as N-bromosuccinimide or N-bromoacetamide, to yield the 5-bromo product 11.2. This compound is then reacted with a dialkyl hydroxy, mercapto or amino-substituted phosphonate 11.3, in which R⁵ is as defined as in Scheme 4, to give the ether, thioether or amine product 11.4. The displacement reaction is conducted in a polar aprotic organic solvent such as dimethylformamide or DMPU, at from 100° C. to about 150° C., in the presence of a base such as triethylamine or cesium carbonate, for example as described in WO 0230930A2, Examples 57-69.

Cyclohexylmethyl-amide 11.6 is reacted with one molar equivalent of N-bromosuccinimide in dichloromethane to yield the 5-bromo product 11.7. This material is then reacted with a dialkyl mercaptoethyl phosphonate 11.8 (Zh. Obschei. Khim., 1973, 43, 2364) and triethylamine at ca 100° C. in a pressure vessel, to produce the thioether 11.9.

Ketal protected 11.11 is brominated with N-bromosuccinimide in ethyl acetate at reflux temperature to yield the bromo compound 11.12 which is reacted with a dialkyl 3-aminophenyl phosphonate 11.13 (J. Med. Chem., 1984, 27, 654) in dimethylformamide at ca. 130° C., using the procedure described in WO 0230930 A2 Example 63, to give the phosphonate 11.14. The product is then reacted with N,N-dimethyloxamide 11.15, (Japanese Patent 540467 18) and dicyclohexylcarbodiimide in dimethylformamide, to yield the amide product 11.16.

Using the above procedures, but employing, in place of the amides 11.6 or 11.11, different amides 11.1, and/or different phosphonates 11.3, the corresponding products 11.4 are obtained.

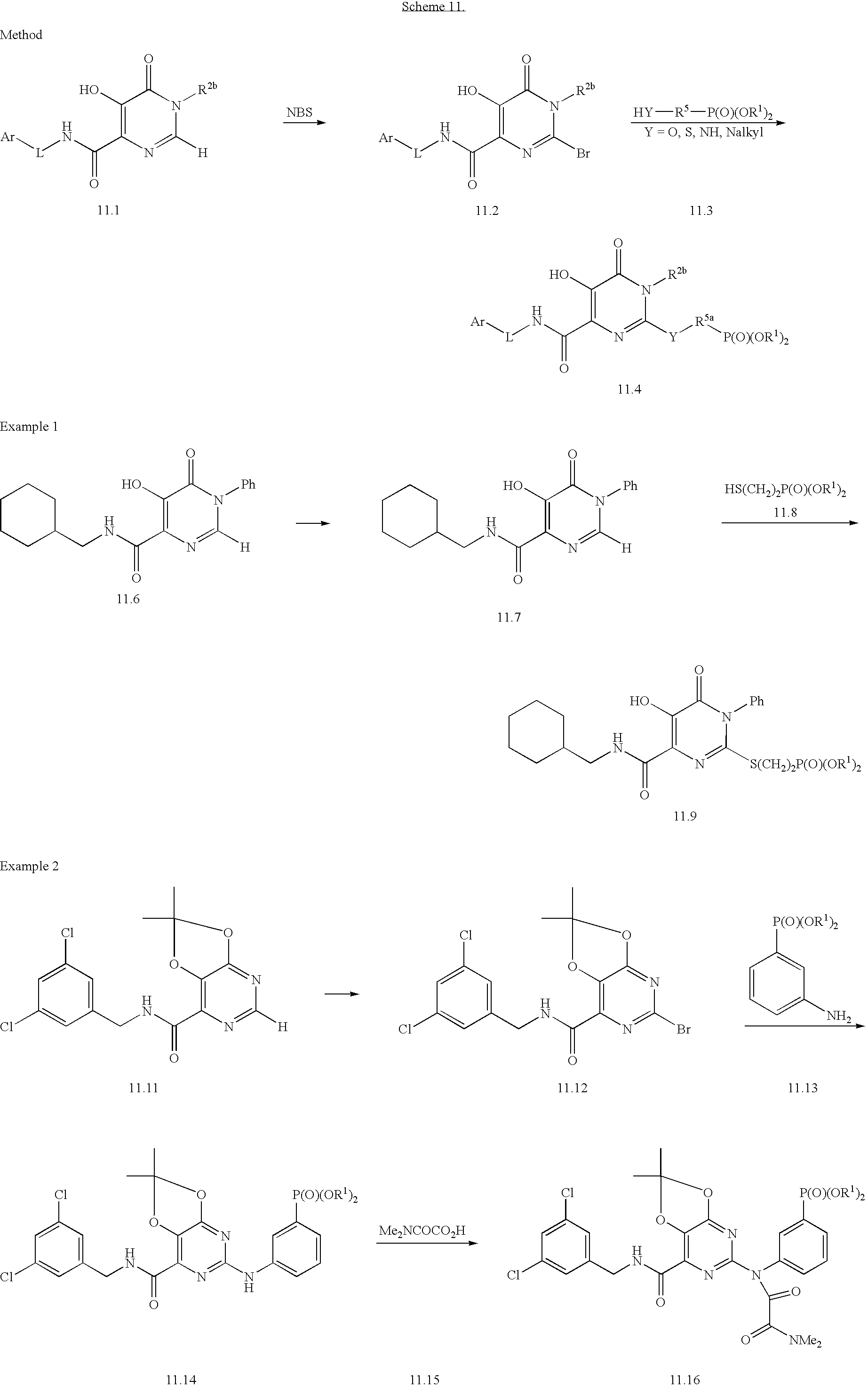

Scheme 12 depicts the preparation of phosphonates Va in which the phosphonate is attached by means of a carbamate linkage. In this procedure, a protected bromophenol 12.1 is reacted, as described in Scheme 11, with an amine 12.2 to give the displacement product 12.3. This compound is then reacted with phosgene, triphosgene, carbonyl diimidazole or a functional equivalent thereof, and a dialkyl hydroxyalkyl phosphonate 12.4, in which R⁵ is as defined in Scheme 4, to yield, after deprotection of the phenol, the carbamate 12.5. Various methods for the preparation of carbamates are described in Scheme 33.

For example, the hydroxyester 12.6 is converted, as described previously, into the amide 12.7. This material is then reacted, in dimethylformamide solution at 100°, with ethylamine and cesium carbonate in dimethylformamide, to afford 5-(tert-butyl-dimethyl-silanyloxy)-2-ethylamino-1-methyl-6-oxo-1,6-dihydro-pyrimidine-4-carboxylic acid [2-(4-fluoro-phenyl)-cyclopropyl]-amide 12.9. The amine is treated with equimolar amounts of a dialkyl hydroxypropyl phosphonate 12.10 (Zh. Obschei. Khim., 1974, 44, 1834) and carbonyldiimidazole in dichloromethane, to prepare, after desilylation, the carbamate phosphonate 12.11.

Using the above procedures, but employing, in place of the amide 12.7, different amides 12.3, and/or different phosphonates 12.4, the corresponding products 12.5 are obtained.

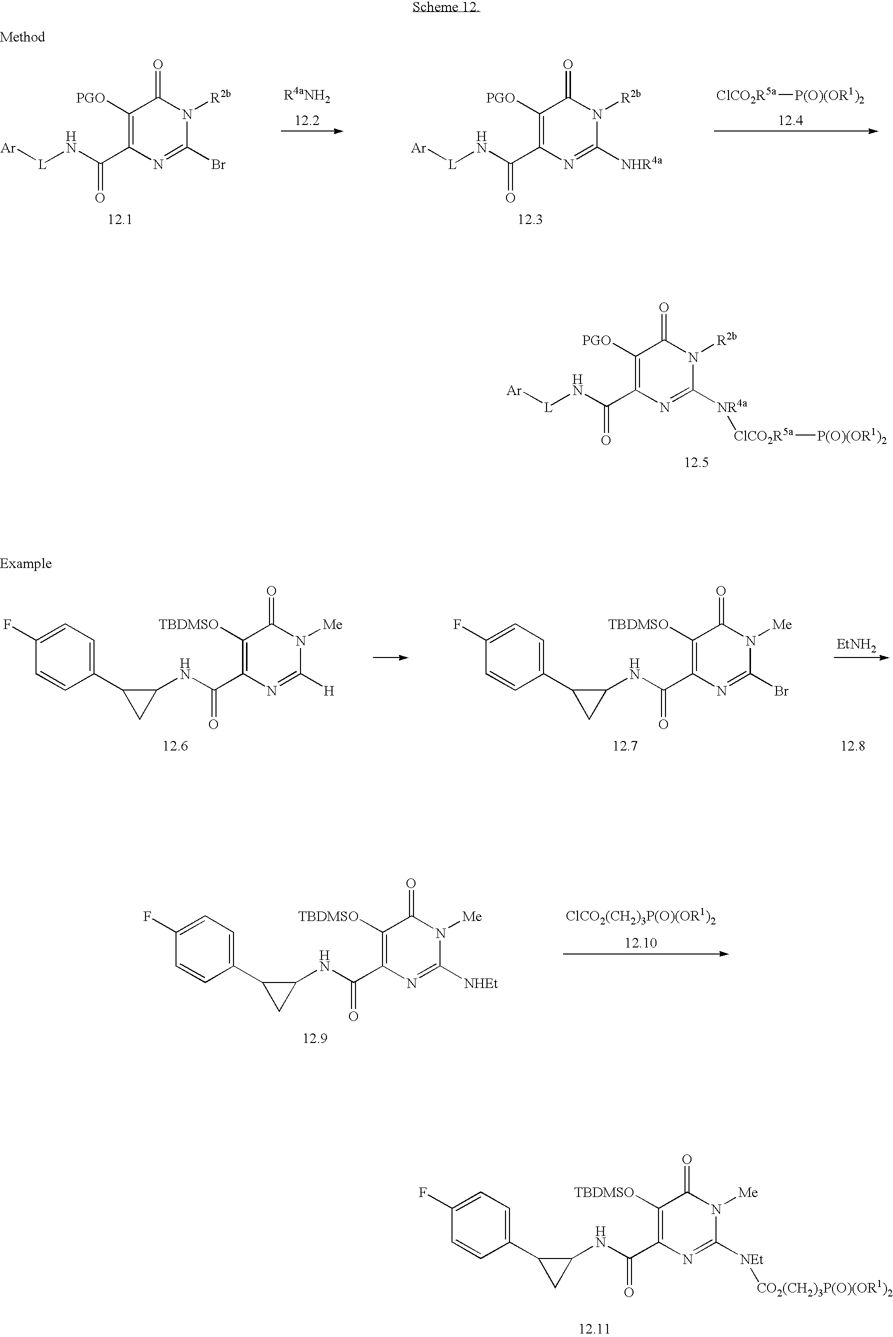

Scheme 13 depicts the preparation of phosphonates Va in which the phosphonate is attached by means of an arylvinyl or arylethyl linkage. In this procedure, a bromophenol 13.1 is protected to give the product 13.2. This compound is then coupled with tributylvinyltin to yield the 5-vinyl product 13.3. The coupling reaction is effected in dimethylformamide solution at ca. 80° C. in the presence of a palladium(0) catalyst, such as tris(dibenzylideneacetone)palladium(0), a triarylphosphine such as tri(2-furyl)phosphine and copper(I) iodide, for example as described in WO 0230930A2, Example 176. The vinyl-substituted product is subjected to a palladium-catalyzed Heck coupling reaction, as described in Scheme 4, with a dibromoaromatic or heteroaromatic compound 13.4, to give the bromoaryl product 13.5. The latter compound is then coupled, as described in Scheme 3, with a dialkyl phosphite 13.6, in the presence of a palladium catalyst, to give the aryl phosphonate 13.7. Deprotection then affords the phenol 13.8. Optionally, the double bond is reduced, for example as described in Scheme 4, to give the saturated analog 13.9.

For example, 5-(tert-butyl-dimethyl-silanyloxy)-1-isopropyl-6-oxo-1,6-dihydro-pyrimidine-4-carboxylic acid 3,5-dichloro-benzylamide 13.10, (WO9944992) is converted, using the methods described above, into 2-bromo-5-(tert-butyl-dimethyl-silanyloxy)-1-isopropyl-6-oxo-1,6-dihydro-pyrimidine-4-carboxylic acid 3,5-dichloro-benzylamide 13.11. The product is coupled, as described above, with tri(n-butyl)vinyltin to produce 2-ethylene-5-(tert-butyl-dimethyl-silanyloxy)-1-isopropyl-6-oxo-1,6-dihydro-pyrimidine-4-carboxylic acid 3,5-dichloro-benzylamide 13.12. This material is then coupled, in dimethylformamide solution at 80° with one molar equivalent of 2,5-dibromothiophene 13.13, in the presence of tetrakis(triphenylphosphine)palladium(0) and triethylamine, to afford 2-[2-(2-bromothiophene)ethylene, 3-isopropyl, 5-tert-butyldimethylsilyloxy, 6-[3,5-dichloro-benzylamide] pyrimidinone 13.14. The product 13.14 is coupled, in the presence of a palladium(0) catalyst and triethylamine, with a dialkyl phosphite 13.15, to afford the phosphonate 13.16. Deprotection, for example by reaction with tetrabutylammonium fluoride in tetrahydrofuran, then yields the phenol 13.17, and hydrogenation of the latter compound in methanol, using 5% palladium on carbon as catalyst, produces the saturated analog 13.18.

Using the above procedures, but employing, in place of the amide 13.11, different amides 13.1, and/or different dibromides 13.4, the corresponding products 13.8 and 13.9 are obtained.

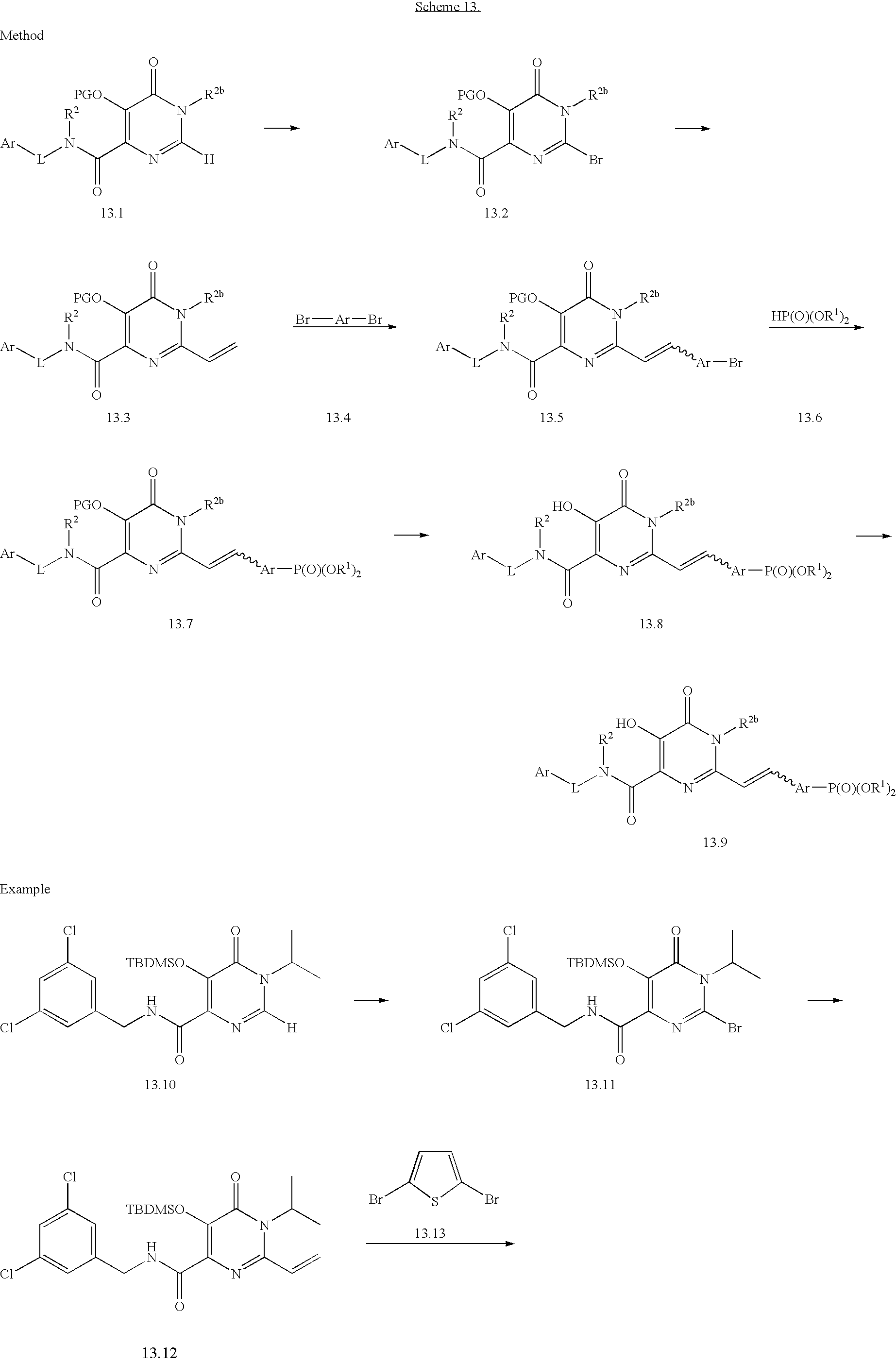

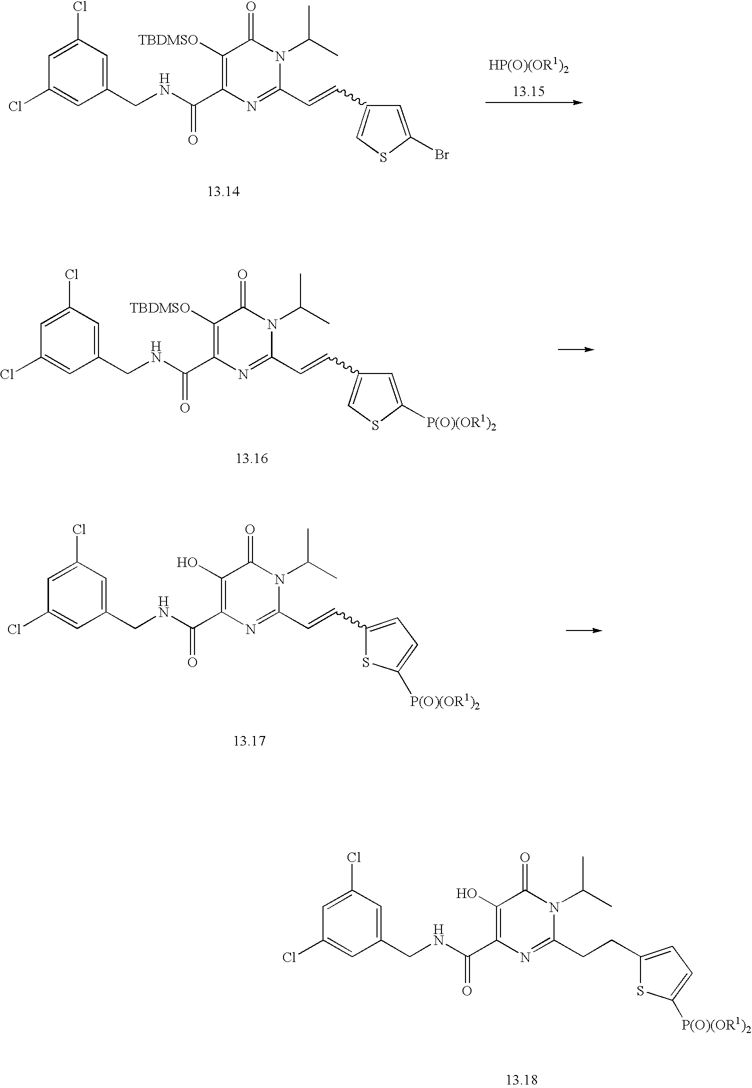

Scheme 14 depicts the preparation of phosphonates IVa in which the phosphonate is attached by means of an acetylenic bond. In this procedure, a phenol 14.1 is reacted, as described in WO 0230930 A2 p. 166 and Example 112, with N-iodosuccinimide in dichloromethane-dimethylformamide, to give the 5-iodo product; protection of the phenolic hydroxyl group then affords the compound 14.2. This material is coupled, as described in WO 0230930 A2 Example 79, in dimethylformamide solution, in the presence of dichlorobis(triphenylphosphine) palladium (II), copper iodide and triethylamine, with a dialkyl ethynyl phosphonate 14.3, in which R^{5a} is as defined in Scheme 4, to give, after deprotection of the phenol, the acetylenic phosphonate 14.4.

Dibenzoyl amide 14.6 is converted into the 2-iodo compound 14.7, as described above, and coupled with a dialkyl propynyl phosphonate 14.8, (Synthesis, (1999), 2027) to yield the acetylenic phosphonate 14.9. After deprotection of the benzoyl groups, the 5,6-dihydroxy-2-methyl-pyrimidine-4-carboxylic acid (cyclopent-3-enylmethyl)-amide phosphonate compound 14.10 is obtained.

Using the above procedures, but employing, in place of the iodoamide 14.7, different iodoamides 14.2, and/or different acetylenic phosphonates 14.3, the corresponding products 14.4 are obtained.

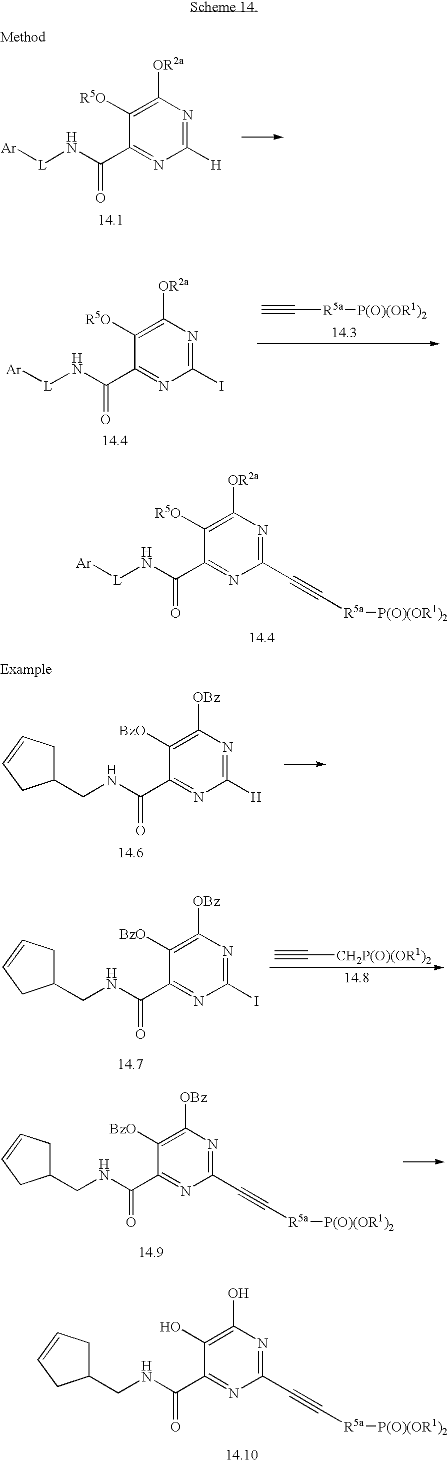

Scheme 15 depicts the preparation of phosphonates Va in which the phosphonate is directly attached to pyrimidinone at the 2-position. In this procedure, a protected 2-bromopyrimidyl 15.1 is coupled, in the presence of a palladium catalyst, as described in Scheme 3, with a dialkyl phosphite 15.2, to give after deprotection the aryl phosphonate 15.3.

For example, 4-oxo-5-(tetrahydro-pyran-2-yloxy)-3-triisopropylsilanyl-3,4-dihydro-pyrimidine-6-carboxylic acid [1-(3-chloro-4-fluoro-phenyl)-1-methyl-ethyl]-amide 15.4, is converted, using the procedures described above, is brominated to give 2-bromo-4-oxo-5-(tetrahydro-pyran-2-yloxy)-3-triisopropylsilanyl-3,4-dihydro-pyrimidine-6-carboxylic acid [1-(3-chloro-4-fluoro-phenyl)-1-methyl-ethyl]-amide 15.5. The product is then coupled, in the presence of tetrakis(triphenylphosphine)palladium(0) and triethylamine, as described in Scheme 3, with a dialkyl phosphite 15.6 (for example, R¹=ethyl), to afford, after desilylation of the phenol, the pyrimidinone 2-phosphonate 15.7 which can be deprotected under acidic conditions to 15.8.

Using the above procedures, but employing, in place of the bromoamide 15.5, different bromoamides 15.1, the corresponding products 15.3 are obtained.

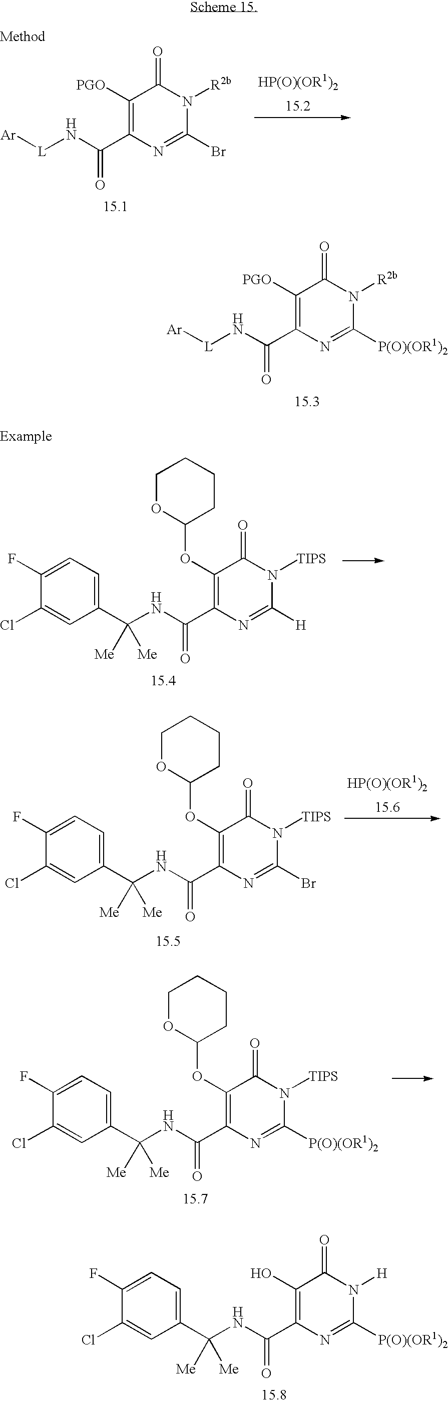

Schemes 16-18 illustrate methods for the preparation of the 2-amino linked phosphonate esters IVa and Va.

Scheme 16 depicts the N-3 sulfonation of 2-phosphonate compounds. In this procedure, 16.1, in which the 5-hydroxyl group is protected, prepared as described in Scheme 11, is reacted with a sulfonyl chloride 16.2 or a sulfonic acid 16.3, in which R^{4a} can be C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, or C₂-C₂₀ substituted heterocycle, to afford sulfonamide 16.4. The reaction between an amine and a sulfonyl chloride, to produce the sulfonamide, is conducted at ambient temperature in an inert solvent such as dichloromethane, in the presence of a tertiary base such as triethylamine. The reaction between a sulfonic acid and an amine to afford a sulfonamide is conducted in a polar solvent such as dimethylformamide, in the presence of a carbodiimide such as dicyclohexyl carbodiimide, for example as described in Synthesis, (1976), 339.

For example, the 5-protected phosphonate diisobutyl ester 16.5, prepared by the methods described above, is reacted in dichloromethane solution with one molar equivalent of ethylsulfonyl chloride 16.6 and triethylamine, to produce 16.7. Desilylation of 16.7 gives {2-[(4-dimethylcarbamoyl-1-ethanesulfonyl-5-hydroxy-6-oxo-1,6-dihydro-pyrimidin-2-yl)-methyl-amino]-ethyl}-phosphonic acid di-sec-butyl ester 16.8.

Using the above procedures, but employing, in place of the amine phosphonate 16.5, different phosphonates 16.1, and/or different sulfonyl chlorides 16.2 or sulfonic acids 16.3, the corresponding products 16.4 are obtained.

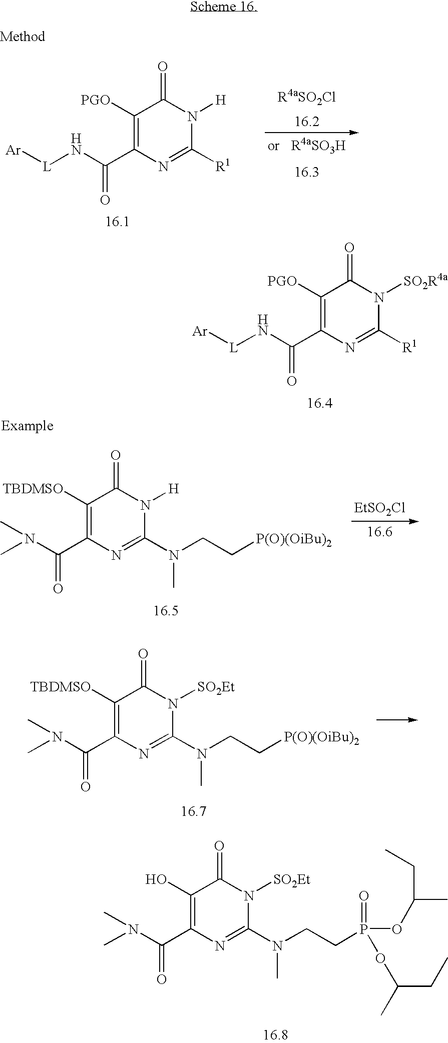

Scheme 17 depicts an alternative method for the preparation of phosphonate esters Va in which the phosphonate group is attached by means of a variable carbon chain from a 2-sulfonamido group. In this procedure, a dialkyl amino-substituted phosphonate 17.1, in which the group R^{5a} is as defined in Scheme 4, is reacted with a sulfonyl chloride 17.2 or sulfonic acid 17.3, as described in Scheme 16, to yield the sulfonamide 17.4. The product is then reacted with a bromoamide 17.5, to prepare the displacement product 17.6. The displacement reaction is performed in a basic solvent such as pyridine or quinoline, at from about 80° to reflux temperature, optionally in the presence of a promoter such as copper oxide, as described in WO 0230930 A2 Example 154.

For example, a dialkyl 4-aminophenyl phosphonate 17.7 (Epsilon) is reacted in dichloromethane solution with one molar equivalent of methanesulfonyl chloride 17.8 and triethylamine, to give the sulfonamide 17.9. The product is then reacted in pyridine solution at reflux temperature with 2-bromo-6-(4-fluoro-benzylcarbamoyl)-3-methyl-6-benzoyloxy-3,4-dihydro-pyrimidin-5-yl ester 17.10, prepared by the methods described above, and copper oxide, to yield the sulfonamide 17.11.

Using the above procedures, but employing, in place of the amine phosphonate 17.7, different phosphonates 17.1, and/or different sulfonyl chlorides 17.2 or sulfonic acids 17.3, the corresponding products 17.6 are obtained.

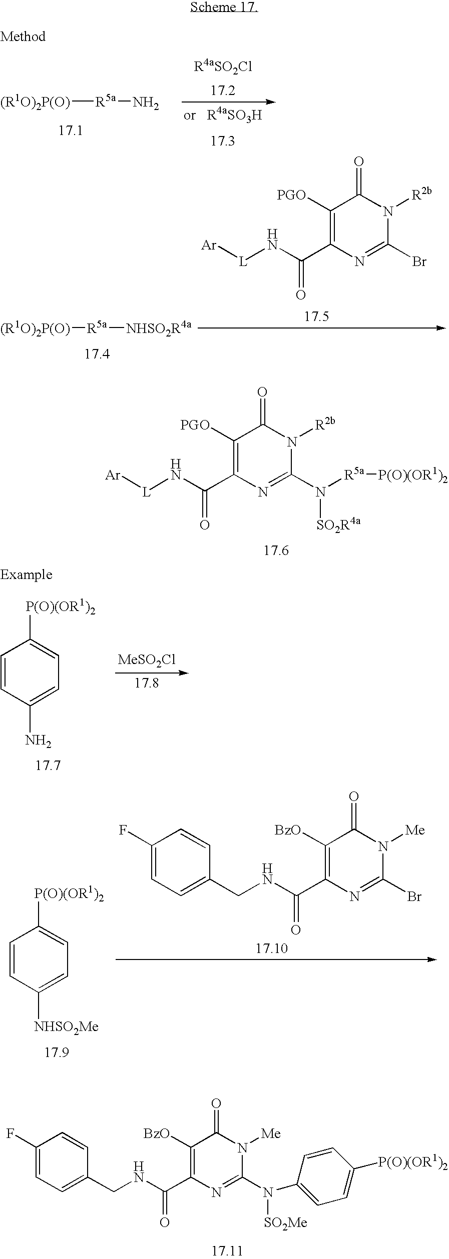

Scheme 18 depicts an alternative method for the preparation of phosphonate esters IVa in which the phosphonate group is attached by means of a variable carbon chain. In this procedure, a phenol-protected 5-bromo substituted amide 18.1 is reacted, as described in Scheme 17, with a sulfonamide 18.2, to give the displacement product 18.3. The product is then reacted with a dialkyl bromoalkyl phosphonate 18.4 to afford, after deprotection of the phenol, the alkylated compound 18.5. The alkylation reaction is performed in a polar aprotic solvent such as dimethylformamide or DMPU, at from ambient temperature to about 100° C., in the presence of a base such as sodium hydride or lithium hexamethyl disilylazide.

For example, benzoic acid 2-bromo-4-hydroxy-6-[1-(3-methoxy-phenyl)-1-methyl-ethylcarbamoyl]-pyrimidin-5-yl ester 18.6, prepared by the methods described above, is reacted in pyridine solution at reflux temperature with one molar equivalent of propanesulfonamide 18.7 and copper oxide, to afford the sulfonamide 18.8. The product is then reacted in dimethylformamide solution with one molar equivalent of a dialkyl bromoethyl phosphonate 18.9 (Aldrich) and lithium hexamethyl disilylazide, to give after debenzoylation, the sulfonamide phosphonate 18.10. The benzoyl protecting group is removed, for example, by reaction with 1% methanolic sodium hydroxide at ambient temperature, as described in Tetrahedron, 26, 803, 1970.

Using the above procedures, but employing, in place of the bromo compound 18.6, different bromo compounds 18.1, and/or different sulfonamides 18.2, and/or different phosphonates 18.4, the corresponding products 18.5 are obtained.

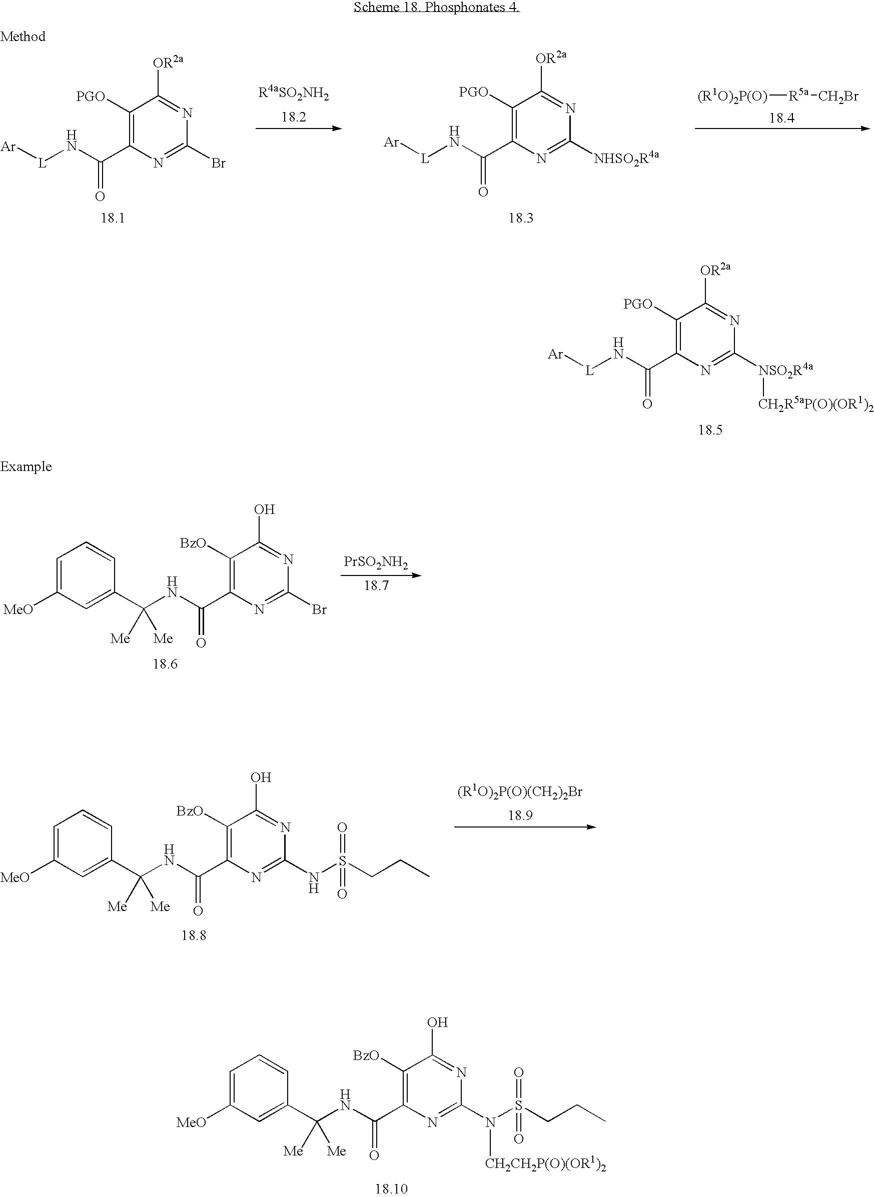

Schemes 19-21 illustrate methods for the preparation of 2-amino linked phosphonate esters IVa and Va.

Scheme 19 illustrates the preparation of phosphonates Va in which the phosphonate group is attached by means of a variable carbon chain. In this procedure, a bromo-substituted sulfonic acid 19.1 is subjected to an Arbuzov reaction with a trialkyl phosphite 19.2 to give the phosphonate 19.3. The Arbuzov reaction is performed by heating the bromo compound with an excess of the trialkyl phosphite at from 100° C. to 150° C., as described in Handbook of Organophosphorus Chem., 1992, 115-72. The resulting phosphonate is then reacted with an amine 19.4, either directly, in the presence of a carbodiimide, or by initial conversion to the sulfonyl chloride, as described in Scheme 16, to afford, after deprotection of the phenolic hydroxyl group, the sulfonamide 19.5.

For example, 3-bromopropanesulfonic acid 19.6 (Sigma) is heated at 130° C. with a trialkyl phosphite 19.7 to give the phosphonate 19.8. The product is then reacted in DMPU solution with 19.9, prepared by the methods described above, in the presence of dicyclohexylcarbodiimide, to give, after desilylation, by reaction with tetrabutylammonium fluoride in tetrahydrofuran, the sulfonamide 19.10.

Using the above procedures, but employing, in place of the bromo sulfonic acid 19.6, different bromosulfonic acids 19.1, and/or different amines 19.4, the corresponding products 19.5 are obtained.

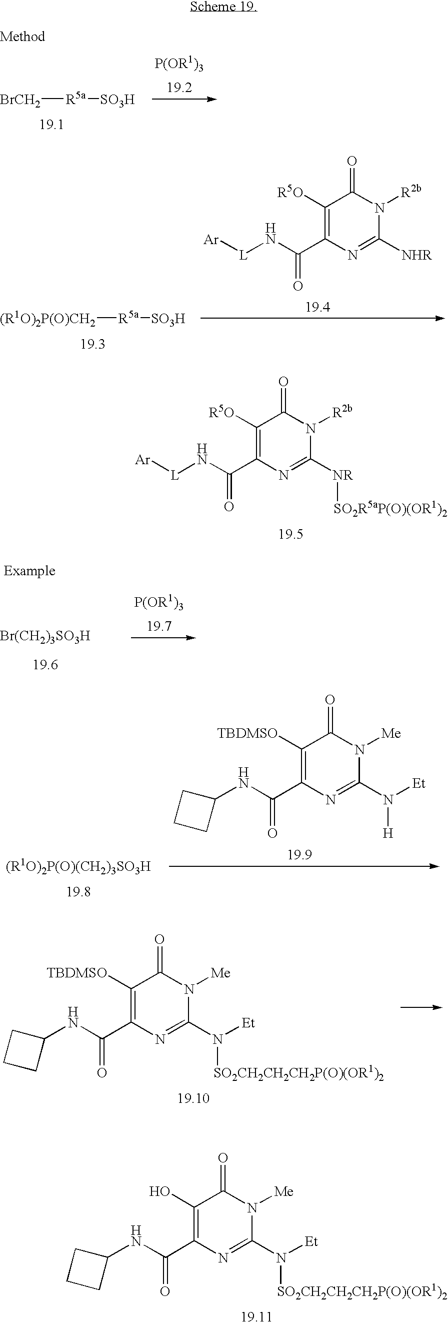

Scheme 20 illustrates the preparation of phosphonates Va in which the phosphonate group is attached by means of a saturated or unsaturated carbon chain and an aromatic or heteroaromatic group. In this procedure, a vinyl-substituted sulfonic acid 20.1 is coupled, in a palladium-catalyzed Heck reaction, as described in Scheme 4, with a dibromoaromatic or heteroaromatic compound 20.2, to yield the sulfonic acid 20.3. The product is then coupled, in the presence of a palladium catalyst, as described in Scheme 3, with a dialkyl phosphite HP(O)(OR¹)₂, to give the phosphonate 20.4. The latter compound is then reacted, as described above, with an amine 20.5, either directly, in the presence of a carbodiimide, or by initial conversion to the sulfonyl chloride, as described in Scheme 16, to afford, after deprotection of the phenolic hydroxyl group, the sulfonamide 20.6. Optionally, the double bond is reduced, either catalytically or chemically, as described in Scheme 4, to afford the saturated analog 20.7.

For example, vinylsulfonic acid 20.8 (Sigma) is coupled, in dioxane solution, in the presence of tetrakis(triphenylphosphine)palladium (0) and potassium carbonate, with 2,5-dibromothiophene 20.9, to form the coupled product 20.10. The product is then reacted in toluene solution at 100° C. with a dialkyl phosphite 20.11, triethylamine and a catalytic amount of tetrakis(triphenylphosphine)palladium (0), to produce the phosphonate 20.12. This material is then reacted, in dimethylformamide solution at ambient temperature, as described above, with 4-fluoro-benzylamide 20.13, prepared by the methods described above, in the presence of dicyclohexylcarbodiimide, to give, after desilylation, using tetrabutylammonium fluoride, the sulfonamide 20.14. Hydrogenation of the double bond, for example using 5% palladium on carbon as catalyst, then yields the saturated analog 20.15.

Using the above procedures, but employing, in place of the sulfonic acid 20.8, different sulfonic acids 20.1, and/or different dibromoaromatic compounds 20.2, and/or different amines 20.5, the corresponding products 20.6 and 20.7 are obtained.

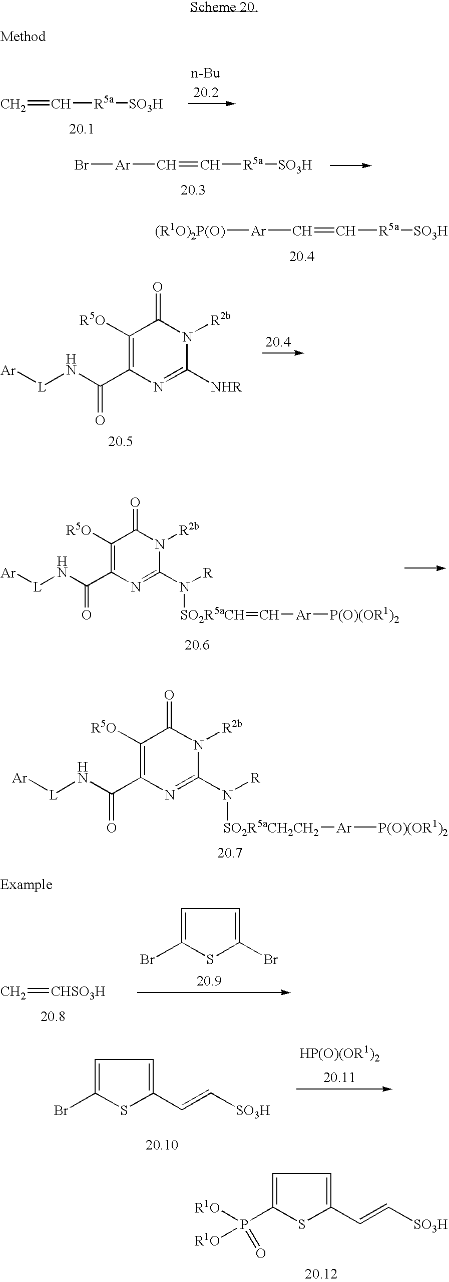

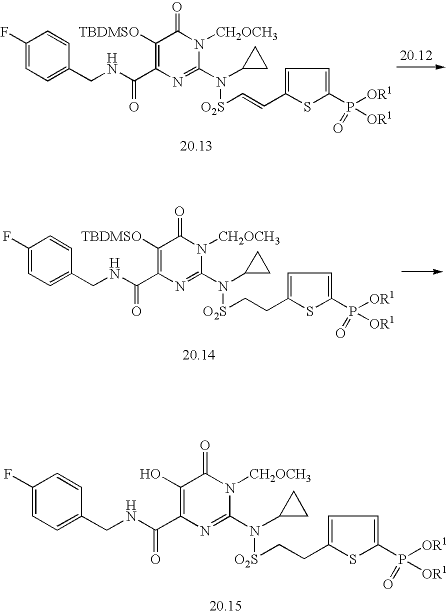

Scheme 21 illustrates the preparation of phosphonates IVa in which the phosphonate group is attached by means of a variable carbon chain. In this procedure, an aliphatic bromo-substituted sulfonic acid 21.1 is subjected to an Arbuzov reaction with a trialkyl phosphite, as described in Scheme 19, to give the phosphonate 21.2. Alternatively, an aryl bromosulfonic acid 21.1 is coupled, as described in Scheme 3, with a dialkyl phosphite, to give the phosphonate 21.2. The product is then reacted with an amine 21.3 to afford the sulfonamide 21.4. The latter compound is then reacted, as described in Scheme 17, with a bromoamide 21.5, to give the displacement product 21.6.

For example, 4-bromobenzenesulfonic acid 21.7 is reacted, as described in Scheme 20, with a dialkyl phosphite to form the phosphonate 21.8. The product is then reacted with phosphoryl chloride to afford the corresponding sulfonyl chloride, and the latter compound is reacted, in dichloromethane solution, in the presence of triethylamine, with 2-methoxyethylamine 21.9, to yield the sulfonamide 21.10. This material is then reacted, in pyridine solution at reflux temperature, with 2-bromo-4,5-dimethoxy-pyrimidine-6-carboxylic acid 4-fluoro-benzylamide 21.11, prepared by the methods described above, and copper oxide, to give the 2-sulfonamide phosphonate 21.12.

Using the above procedures, but employing, in place of the sulfonic acid 21.7, different sulfonic acids 21.1, and/or different amines 21.3, and/or different bromo compounds 21.5, the corresponding products 21.6 are obtained. 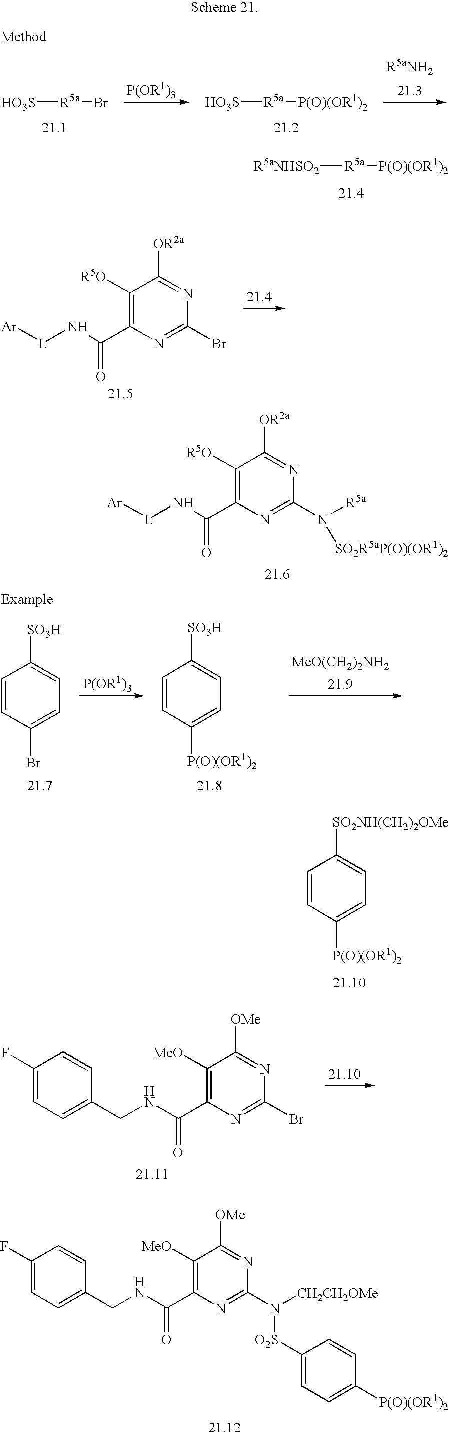
Preparation of Phosphonate Esters IVa and Va.

Scheme 22 depicts the preparation of phosphonate esters IVa in which the phosphonate group is attached by means of an cyclic sulfonamide group at the 2-amino position. In this procedure, a cyclic sulfonamide 22.1, where m and n are independently 1, 2, 3, 4, 5, or 6, and incorporating a secondary amine, is coupled, as described in Scheme 5, with a dialkyl carboxy-substituted phosphonate 22.2 to produce the amide 22.3. The product is then reacted with a bromoamide 22.4 to afford the displacement product 22.5.

Alternatively, the cyclic sulfonamide 22.1 is protected to give the analog 22.6. Sulfonamides are protected, for example, by conversion into the N-acyloxymethyl derivatives, such as the pivalyloxymethyl derivative or the benzoyloxymethyl derivative, by reaction with the corresponding acyloxymethyl chloride in the presence of dimethylaminopyridine, as described in Bioorg. Med. Chem. Lett., 1995, 5, 937, or by conversion into the carbamate derivative, for example the tert. butyl carbamate, by reaction with an alkyl, aryl or aralkyl chloroformate, in the presence of a base such as triethylamine, as described in Tet. Lett., 1994, 35, 379. The protected sulfonamide is reacted with a dialkyl bromoalkyl phosphonate 22.7 to form the alkylated product 22.8. The alkylation reaction is effected as described in Scheme 8. The product is then deprotected to yield the sulfonamide 22.9. Deprotection of pivalyloxymethyl amides is effected by treatment with trifluoroacetic acid; deprotection of benzyloxymethyl amides is effected by catalytic hydrogenation, as described in Protective Groups in Organic Synthesis, by T. W. Greene and P. G. M. Wuts, Wiley, Second Edition 1990, p. 398. Sulfonamide carbamates, for example the tert. butyl carbamate, are deprotected by treatment with trifluoroacetic acid. The sulfonamide 22.9 is then reacted with the bromoamide 22.10 to give the displacement product 22.11.

For example, [1,2,5]thiadiazepane 1,1-dioxide 22.11A (WO 0230930A2 p. 321) is reacted in dioxane solution with equimolar amounts of a dialkyl 3-carboxypropyl phosphonate 23.12, (Epsilon) and dicyclohexylcarbodiimide, to produce the amide 22.13. This material is reacted in pyridine solution at reflux temperature with 2-bromo-3-methyl-4-oxo-5-triisopropylsilanyloxy-3,4-dihydro-pyrimidine-6-carboxylic acid 4-fluoro-benzylamide 22.14, prepared by the methods described above, and copper oxide, to afford the displacement product 22.15.

As a further example, the sulfonamide 22.11A is reacted in dichloromethane with one molar equivalent of t-Boc anhydride, triethylamine and dimethylaminopyridine, to give 1,1-dioxo-[1,2,5]thiadiazepane-2-carboxylic acid tert-7butyl ester 22.16. The product is then reacted at ambient temperature in dimethylformamide solution with a dialkyl 4-bromomethyl benzyl phosphonate 22.17, (Tetrahedron, 1998, 54, 9341) and potassium carbonate, to yield the alkylation product 22.18. The BOC group is removed by treatment with trifluoroacetic acid to give the sulfonamide 22.19, and this material is reacted, as described above, with 2-bromo-3,4-dihydroxy-pyrimidine-6-carboxylic acid 3-fluoro-benzylamide 22.20, prepared by the methods described above, to afford the displacement product 22.21.

Using the above procedures, but employing, in place of the sulfonamide 22.11A, different sulfonamides 22.1, and/or different carboxylic acids 22.2 or alkyl bromides 22.7, and/or different bromides 22.4, the corresponding products 22.5 and 22.11 are obtained.

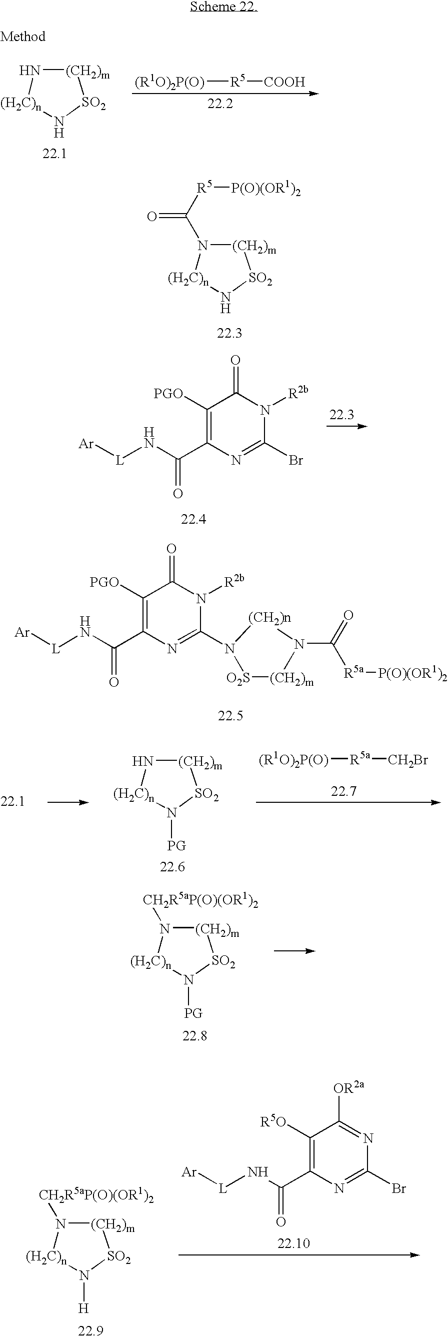

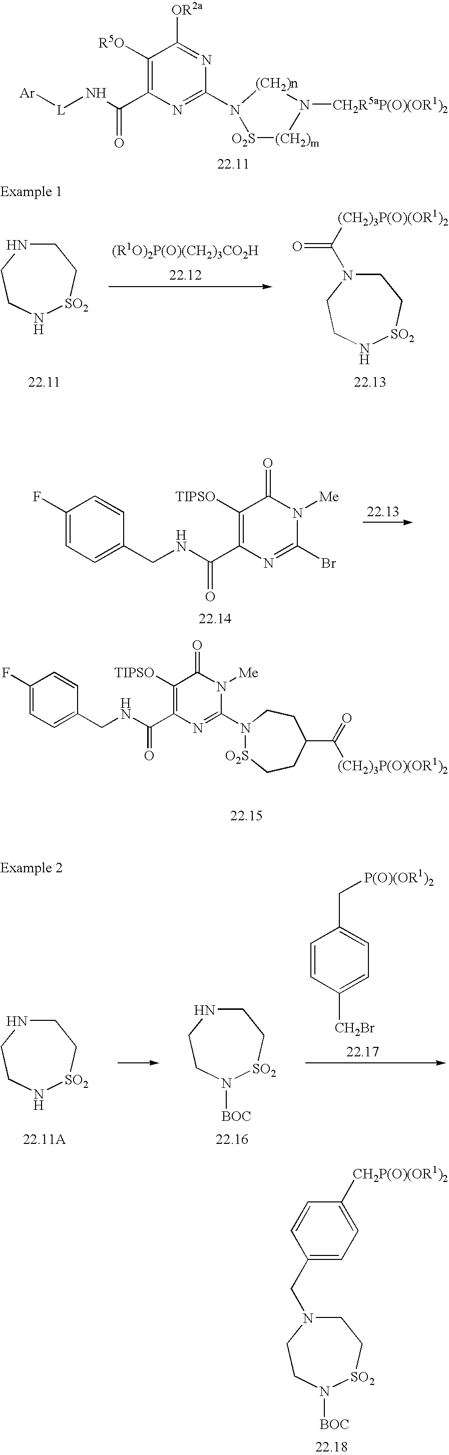

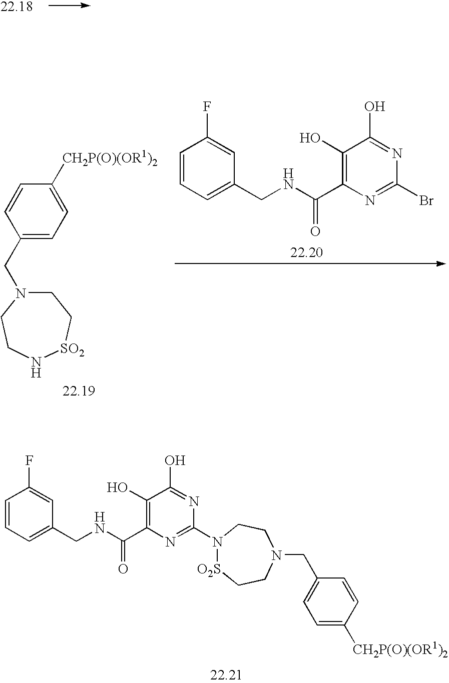

Scheme 23 depicts the preparation of phosphonates Va in which the phosphonate group is attached by means of an aryl or heterocycle group. In this procedure, a bromoaryl-substituted cyclic sulfonamide, prepared as described in J. Org. Chem., (1991), 56, 3549, from the corresponding bromoaryl or bromoheterocycle acetic acid and a vinyl sulfonic ester, is coupled, as described in Scheme 3, with a dialkyl phosphite to afford the phosphonate 23.2. The product is then reacted, as described above, with a bromoamide 23.3 to yield the displacement product 23.4.

For example, 4-(4-bromo-phenyl)-[1,2]thiazinane 1,1-dioxide 23.5 (J. Org. Chem., 1991, 56:3549) is reacted in dimethylformamide solution with a dialkyl phosphite 23.6 and tetrakis(triphenylphosphine)palladium(0), to give the phosphonate 23.7. The product is then reacted with 2-bromo-3-(2-methoxy-ethyl)-4-oxo-5-triisopropylsilanyloxy-3,4-dihydro-pyrimidine-6-carboxylic acid (5-fluoro-indan-1-yl)-amide 23.8, prepared by the methods described above, to give the phosphonate 23.9.

Using the above procedures, but employing, in place of the sulfonamide 23.5, different sulfonamides 23.1, and/or different bromo compounds 23.3, the corresponding products 23.4 are obtained.

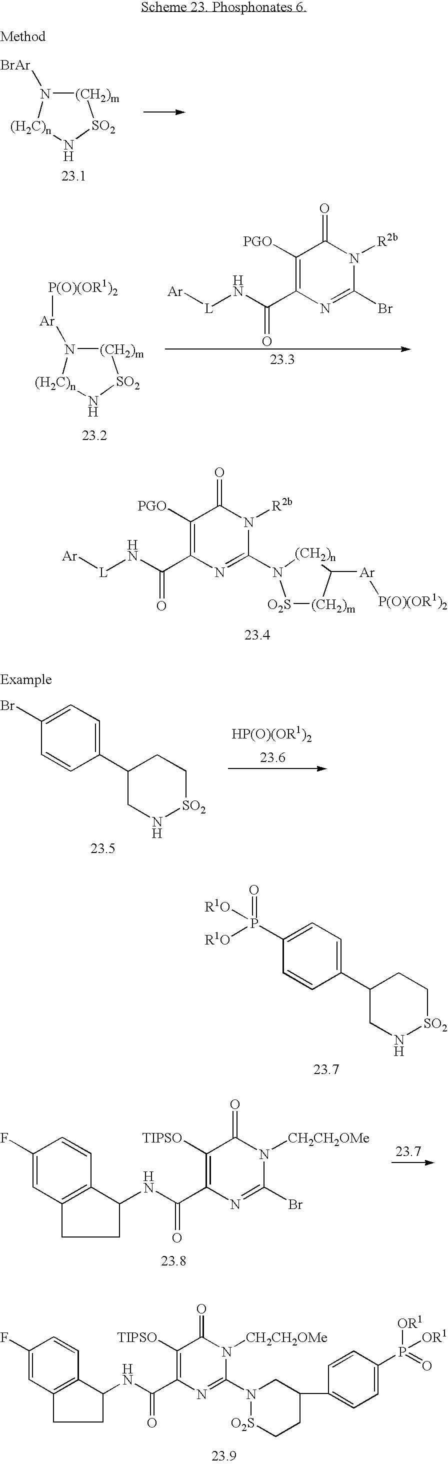

Scheme 24 depicts the preparation of phosphonates IVa in which the phosphonate group is attached by means of an amide linkage. In this procedure, a carboxy-substituted cyclic sulfonamide 24.1 is coupled with an amino-substituted dialkyl phosphonate 24.2, as described in Scheme 5, to give the amide 24.3. The product is then reacted with the bromoamide 24.4 to afford the displacement product 24.5.

For example, 1,1-dioxo-[1,2]thiazinane-3-carboxylic acid 24.6 (Izvest. Akad. Nauk SSSR Ser. Khim., 1964, 9, 1615) is reacted in dimethylformamide solution with equimolar amounts of an amino-substituted butyl phosphonate 24.7 (Acros) and dicyclohexylcarbodiimide, to afford the amide 24.8. The latter compound is then condensed with 2-bromo-5,6,7,8,8a,10a-hexahydro-9,10-dioxa-1,3-diaza-anthracene-6-carboxylic acid [1-(3-chloro-4-fluoro-phenyl)-ethyl]-amide 24.9, prepared by the methods described above, to give the product 24.10.

Using the above procedures, but employing, in place of the sulfonamide 24.6, different sulfonamides 24.1, and/or different bromo compounds 24.4, the corresponding products 24.5 are obtained.

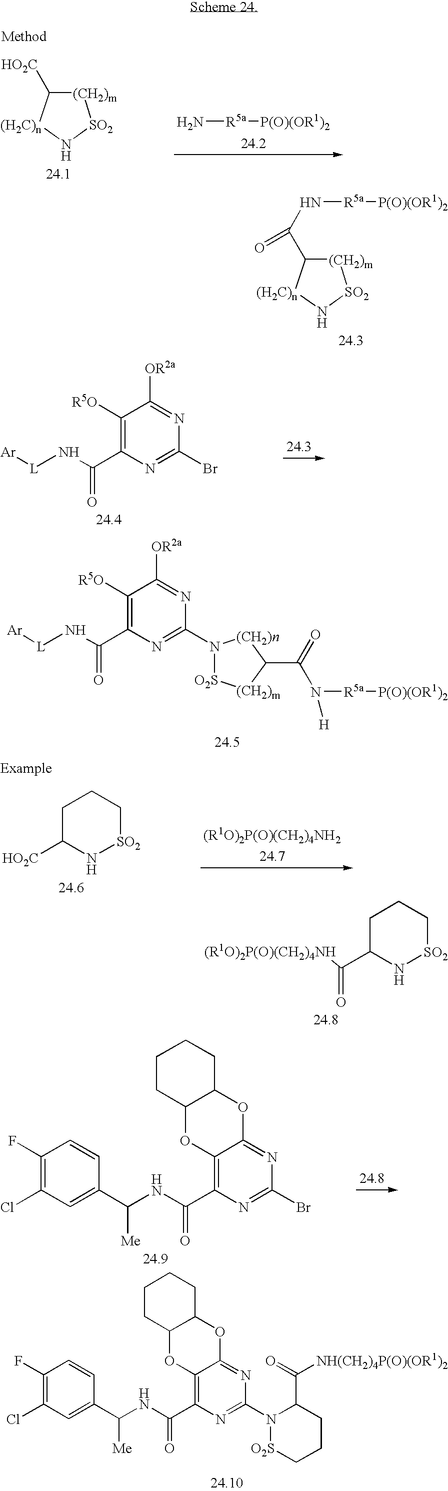

Schemes 25-27 illustrate methods for the preparation of the phosphonate esters IVa and Va in which the phosphonate is attached by means of a carbon link or a variable carbon chain incorporating a heteroatom. In these procedures, for example, a tolyl-substituted pyrimidine 25.1 is reacted with a free radical brominating agent such as N-bromosuccinimide to prepare the bromomethyl derivative 25.3. The benzylic bromination reaction is performed at reflux temperature in an inert organic solvent such as hexachloroethane or ethyl acetate, optionally in the presence of an initiator such as dibenzoyl peroxide. The bromomethyl compound 25.3 is then reacted with a trialkyl phosphite in an Arbuzov reaction, as described in Scheme 19, to give, after deprotection of the phenolic hydroxyl group, the phosphonate 25.4.

Alternatively, the benzylic bromide 25.3 is reacted with a dialkyl hydroxy, mercapto or amino-substituted phosphonate 25.5, to afford, after deprotection of the phenolic hydroxyl group, the displacement product 25.6. The displacement reaction is effected at from ambient temperature to about 100° C., in a polar organic solvent such as dimethylformamide or DMPU, in the presence of a suitable base such as sodium hydride or lithium hexamethyldisilazide, for instances in which Y is O, or cesium carbonate or triethylamine for instances in which Y is S or N.

For example 6-p-tolyl-2,3,3a,9a-tetrahydro-1H-4,9-dioxa-5,7-diaza-cyclopenta[b]naphthalene-8-carboxylic acid 4-fluoro-benzylamide 25.8 is reacted with one molar equivalent of N-bromosuccinimide in ethyl acetate at reflux, to afford the bromomethyl analog 25.9. This product is reacted with a dialkyl hydroxyethyl phosphonate 25.11 (Epsilon) and sodium hydride in dimethylformamide at 80°, to yield, after desilylation, the phosphonate 25.12. Alternatively, the bromomethyl compound 25.9 is reacted at 120° C. with a trialkyl phosphite, to obtain, after desilylation, the phosphonate 25.10.

Using the above procedures, but employing, in place of the anhydride 25.7, different anhydrides 25.1, and/or different phosphonates 25.5, the corresponding products 25.4 and 25.6 are obtained.

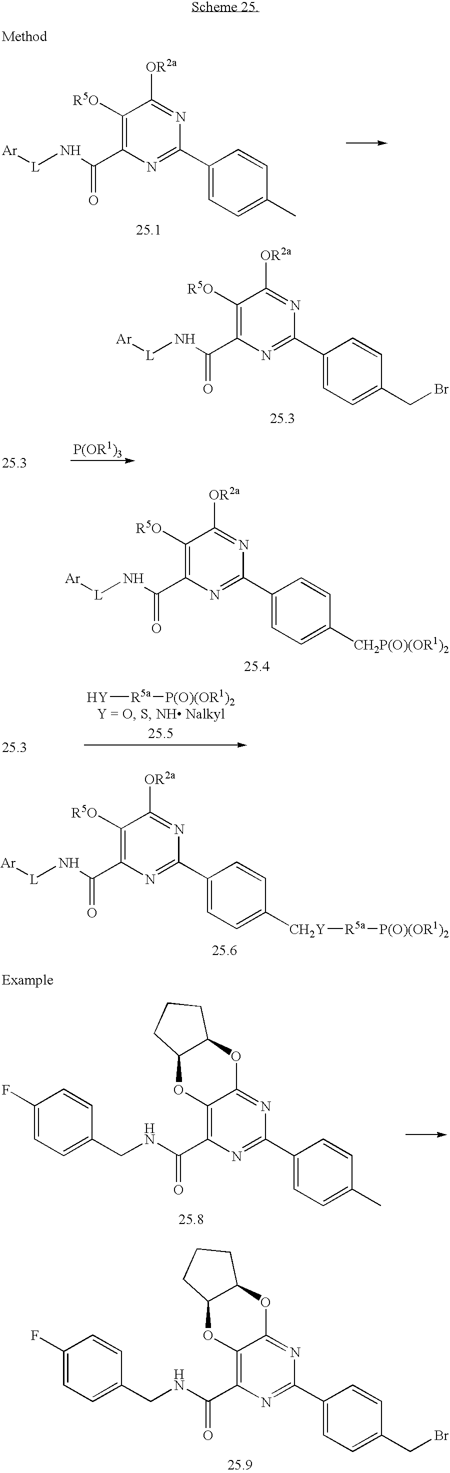

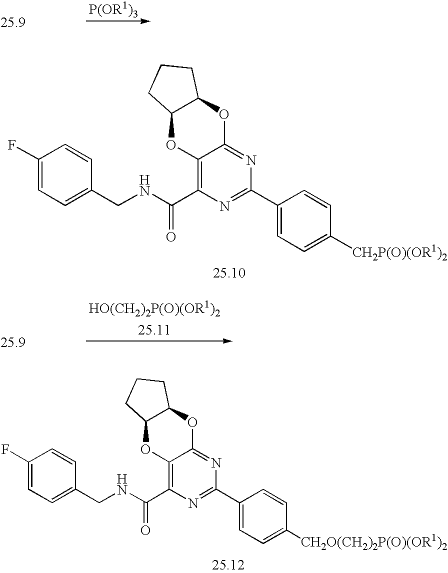

Scheme 26 illustrates the preparation of phosphonate esters Va in which the phosphonate is attached by means of an aminomethyl linkage through the 2-position. In this procedure, a bromomethyl-substituted bicyclic amide 26.1a, prepared as described in Scheme 25, is oxidized to the corresponding aldehyde 26.1. The oxidation of halomethyl compounds to aldehydes is described, for example, in Comprehensive Organic Transformations, by R. C. Larock, VCH, 1989, p. 599ff. The transformation is effected by treatment with dimethylsulfoxide and base, optionally in the presence of a silver salt, or by reaction with trimethylamine N-oxide or hexamethylene tetramine. The aldehyde 26.1 is then reacted with a dialkyl amino-substituted phosphonate 26.2 in a reductive amination reaction (H-=reducing agent), as described in Scheme 9, to yield, after deprotection of the phenolic hydroxyl group, the aminomethyl product 26.3.

For example, 5-benzyloxymethoxy-2-(4-bromomethyl-phenyl)-4-oxo-3,4-dihydro-pyrimidine-6-carboxylic acid 3,5-dichloro-benzylamide 26.4, prepared from the anhydride 25.7, using the methods described in Scheme 25, is reacted with dimethylsulfoxide and 2,4,6-collidine at 90°, as described in J. Org. Chem. (1986) 51:1264, to afford the aldehyde 26.5. The product is then reacted with one molar equivalent of a dialkyl aminoethyl phosphonate 26.6 (Epsilon) and sodium triacetoxyborohydride to produce, after desilylation, the phosphonate 26.7.

Using the above procedures, but employing, in place of the bromomethyl compound 26.4, different bromomethyl compounds 25.3, and/or different phosphonates 26.2, the corresponding products 26.3 are obtained.

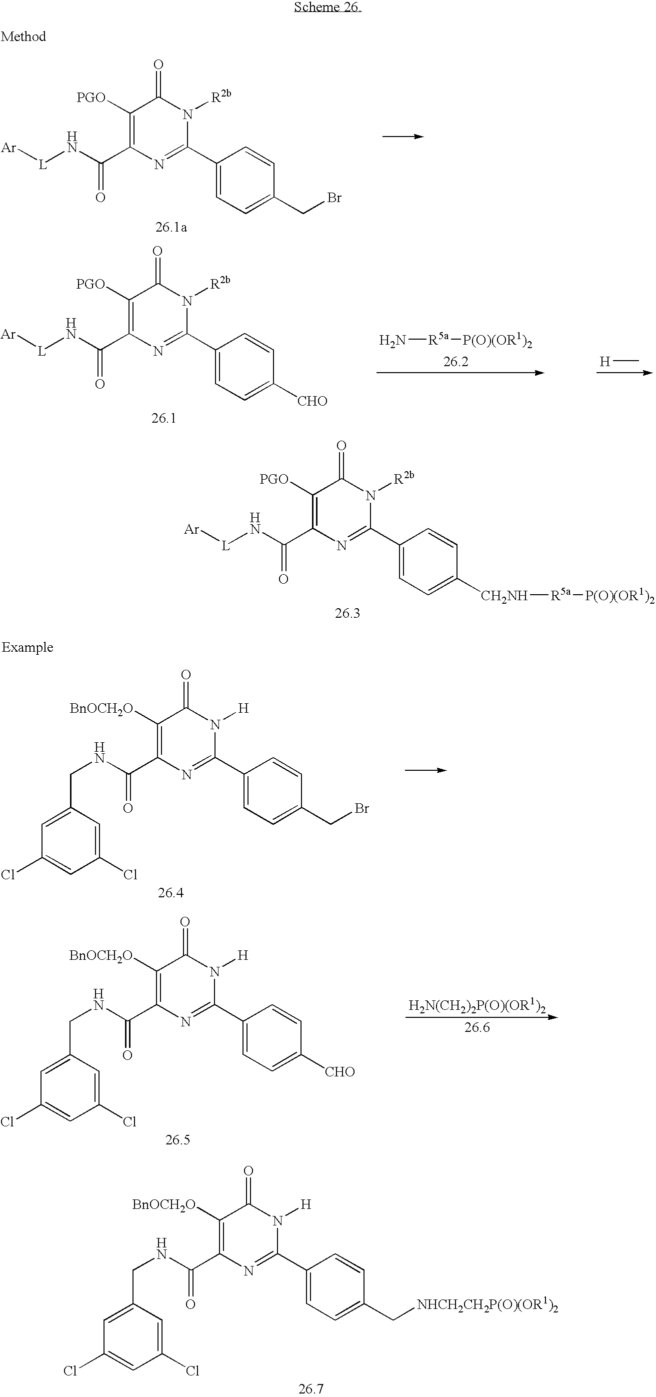

A reductive amination procedure can also be employed to attach a phosphonate ester through an amino linker. 1-Methyl-6-oxo-2-(2-oxo-ethyl)-5-triisopropylsilanyloxy-1,6-dihydro-pyrimidine-4-carboxylic acid 4-fluoro-benzylamide 26.8, prepared by the method of WO 03/03577 at page 96 can be reductively aminated by amino phosphonate reagents, 26.9, 26.10, and 26.11 to give 26.12, 26.13, and 26.14, respectively, after desilylation with tetrabutylammonium fluoride (TBAF) (Scheme 26a). As with the previous examples herein, R¹ may be further converted to other phosphorus substituents, e.g. X and Y. Embodiments of phosphonate substituent X include OPh, OAr, OCH₂CF₃, and NHR, where R is the residue of an amino acid. Embodiments of phosphonate substituent Y include a lactate ester or a phosphonamidate.

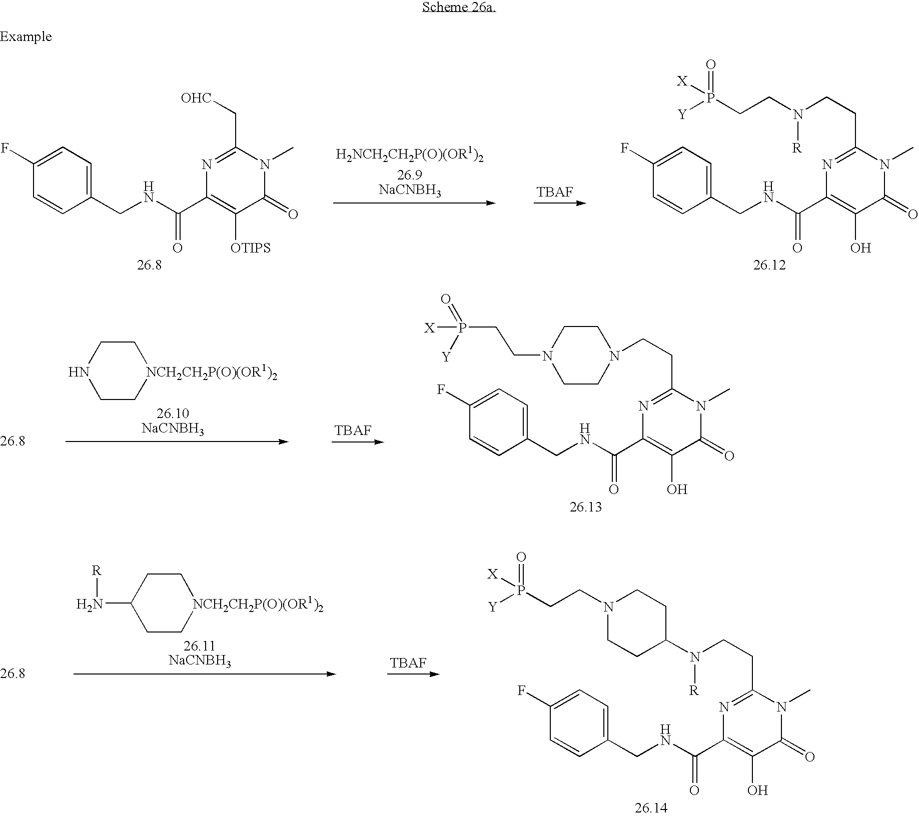

6-Oxo-1-(2-oxo-ethyl)-5-triisopropylsilanyloxy-1,6-dihydro-pyrimidine-4-carboxylic acid 4-fluoro-benzylamide 26.15, prepared from 1-allyl-5-(2,2-dimethyl-propionyloxy)-6-oxo-1,6-dihydro-pyrimidine-4-carboxylic acid methyl ester 26.16 (piv=pivalate, (CH₃)₃CC(O)—) by the method of WO 03/03577 at page 110 can be reductively aminated by amino phosphonate reagents, 26.9, 26.10, and 26.11 to give 26.17, 26.18, and 26.19, respectively after desilylation with TBAF (Scheme 26b).

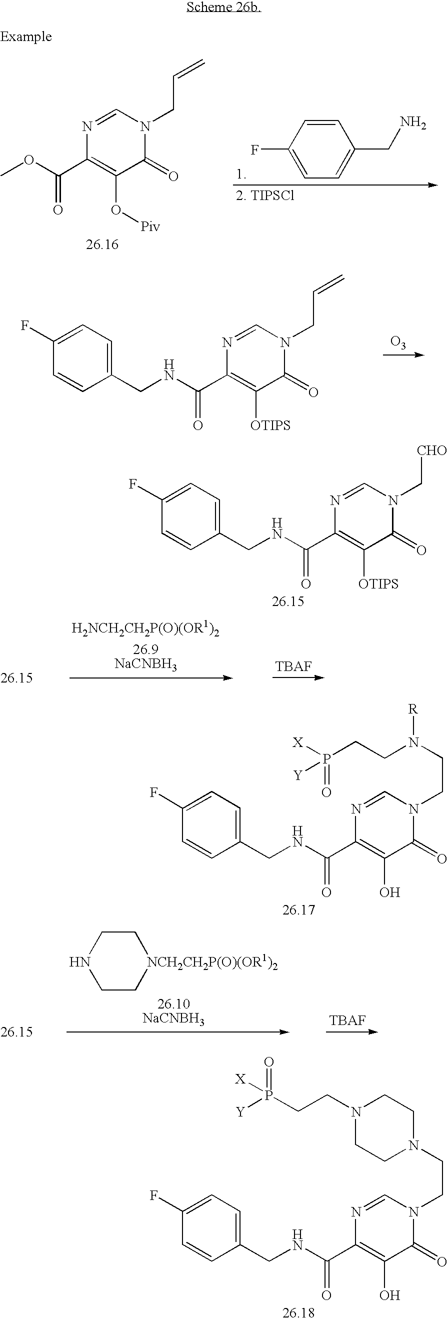

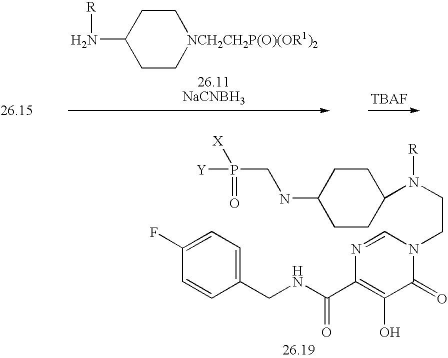

Scheme 27 illustrates the preparation of phosphonate esters IVa in which the phosphonate is attached by coupling a carboxylic acid with an amino phosphonate reagent to form an amide linkage. In this procedure, an aldehyde 27.1, or 26.1 from Scheme 26, is oxidized to the corresponding carboxylic acid 27.2. The conversion of aldehydes to the corresponding carboxylic acids is described in Comprehensive Organic Transformations, by R. C. Larock, VCH, 1989, p. 838. The reaction is effected by the use of various oxidizing agents such as, for example, potassium permanganate, ruthenium tetroxide, silver oxide or sodium chlorite. The resultant carboxylic acid 27.2 is then coupled, as described in Scheme 5, with a dialkyl amino-substituted phosphonate 27.3, to yield the amide 27.4.

For example, 2-(4-formyl-phenyl)-4-methoxy-5-triisopropylsilanyloxy-pyrimidine-6-carboxylic acid (cyclohex-3-enylmethyl)-amide 27.5 is reacted with silver oxide in aqueous sodium hydroxide, as described in Org. Syn. Coll. Vol. 4, 919, 1963, to afford the carboxylic acid 27.6. The latter compound is then reacted in dioxane solution at ambient temperature with equimolar amounts of a dialkyl aminomethyl phosphonate 27.7 (Interchim) and dicyclohexylcarbodiimide, to give, after desilylation, the amide phosphonate 27.8.

Using the above procedures, but employing, in place of the aldehyde 27.5, different aldehydes 26.1, and/or different phosphonates 27.3, the corresponding amides 27.4 are obtained. For example, 5,6-dihydroxy-pyrimidine-2,4-dicarboxylic acid 4-methyl ester 27.9, prepared by the method of WO 03/035077, p. 85, may be converted to the 4-fluorobenzyl amide 27.10 with 4-fluorobenzylamine (Scheme 27a), and the carboxylic acid group coupled with a plethora of amines, including 26.9, 26.10, and 26.11 to give 27.11, 27.12, and 27.13, respectively (Scheme 27b).

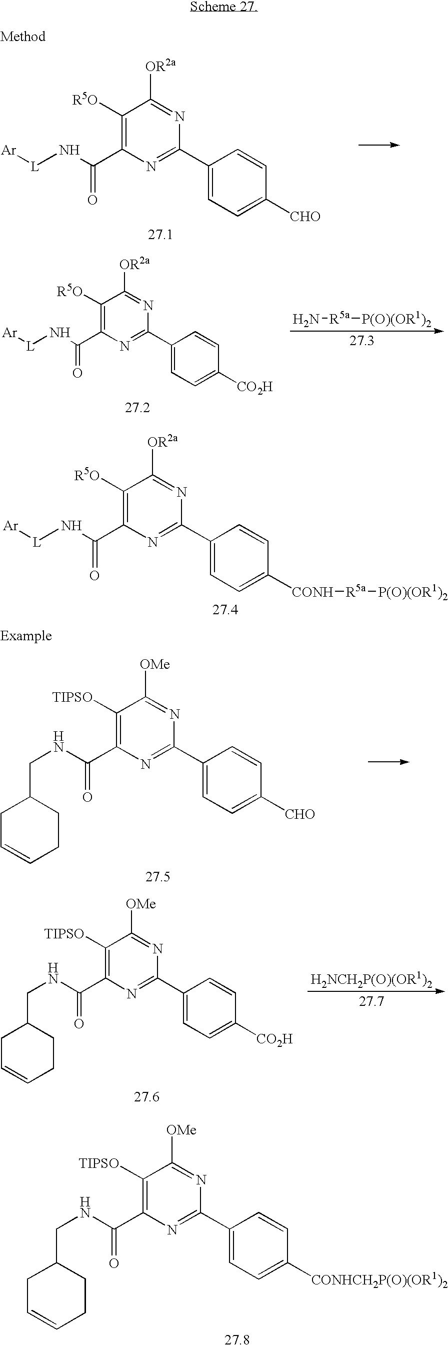

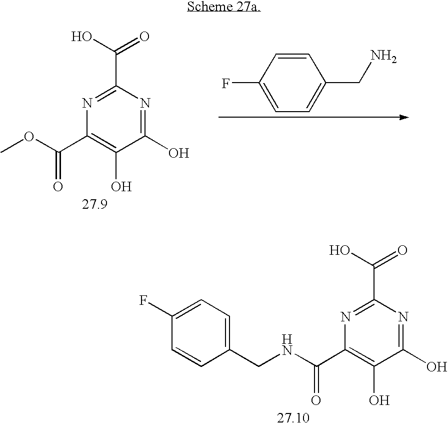

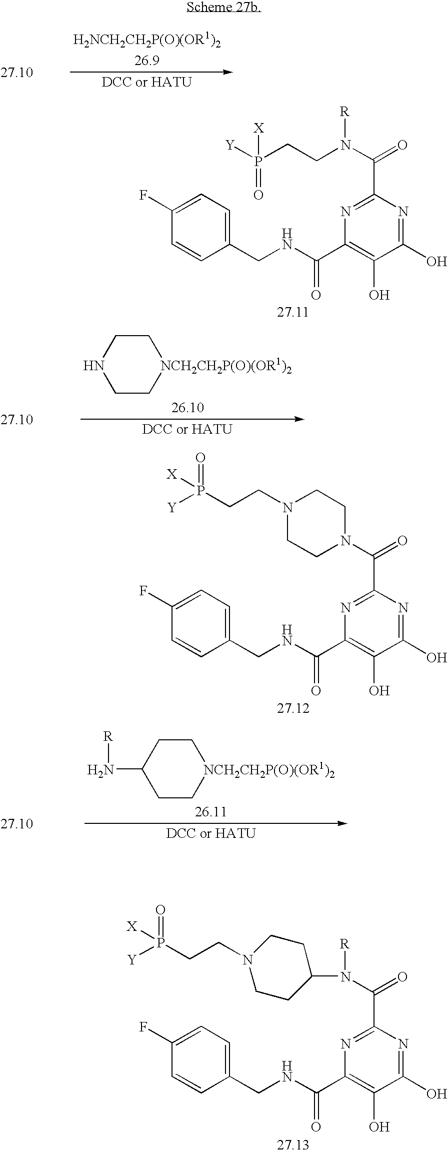

Scheme 28 illustrates the preparation of phosphonate esters IVb in which the phosphonate is attached by means of a heteroatom O or S and a variable carbon link at the 4-position. In this procedure, the 5-hydroxyl protected methyl ester 28.1 is subjected to a Mitsunobu reaction, as described in Scheme 7, with a dialkyl hydroxy or mercapto-substituted phosphonate 28.8, to produce the ether or thioether phosphonate 28.9. This compound is then reacted, as described in Scheme 3, with the amine Ar-L-NR³H, to give amide 28.10. Alternatively, 28.1 is reacted with a dialkyl bromoalkyl-substituted phosphonate 28.5, as described in Scheme 6, to yield the ether 28.6. The latter compound is then transformed, as described above, into the amide 28.7.

In other embodiments, Scheme 28a shows 5-hydroxy-3-methyl-4-oxo-2-p-tolyl-1,6-dihydro-pyrimidine-6-carboxylic acid benzylamide 28.11 reacting with a dialkyl 2-mercaptoethyl phosphonate 28.18 (Zh. Obschei. Khim., (1973), 43, 2364), diethylazodicarboxylate and triphenylphosphine to give thioether 28.12. 3-Ethyl-5-hydroxy-4-oxo-2-p-tolyl-3,4-dihydro-pyrimidine-6-carboxylic acid [1-(4-fluoro-phenyl)-cyclopropyl]-amide 28.13 is reacted with a dialkyl bromomethyl phosphonate 28.15 (Lancaster) and potassium carbonate, to produce the phosphonate 28.16. 5-Hydroxy-4-oxo-3-propyl-2-p-tolyl-3,4-dihydro-pyrimidine-6-carboxylic acid (5-sulfamoyl-naphthalen-2-ylmethyl)-amide 28.17 is alkylated with 2-chloroethyl dialkylphosphonate reagent 28.19 to give phosphonate pyrimidinone 28.20.

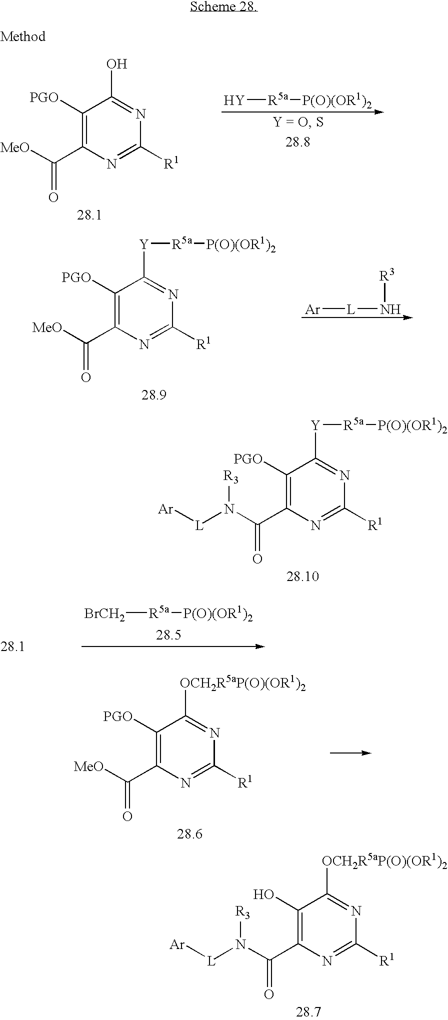

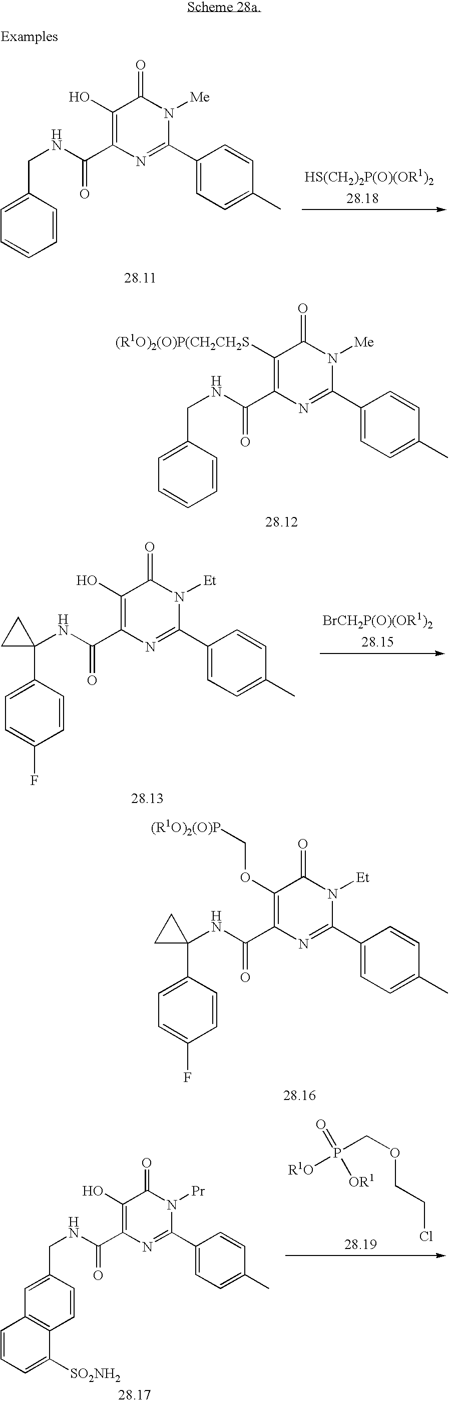

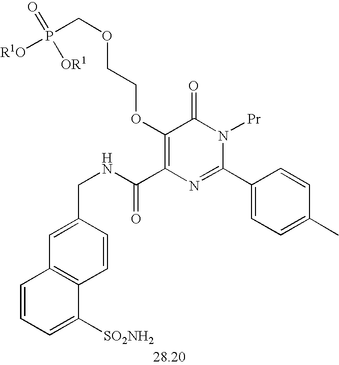

Scheme 29 illustrates the preparation of phosphonate esters IVa in which the phosphonate is attached either directly, or by means of a saturated or unsaturated carbon chain at the 2-position. In this procedure, a bromo-substituted anhydride 29.1 is converted, as described above, into the phenol-protected amide 29.2. The product is then subjected to a Heck coupling reaction, in the presence of a palladium (0) catalyst, as described in Scheme 4, with a dialkyl alkenyl phosphonate 29.3, to afford the phosphonate 29.4. Optionally, the olefinic bond is reduced, as described in Scheme 4, to yield the saturated analog 29.5.

Alternatively, the bromo-substituted amide 29.1 is coupled, as described in Scheme 3, with a dialkyl phosphite, in the presence of a palladium (0) catalyst, to generate, after deprotection of the phenolic hydroxyl group, the amide phosphonate 29.6.

For example, 2-bromo-4,5-dihydroxy-pyrimidine-6-carboxylic acid 4-trifluoromethyl-benzylamide 29.8. This compound is then reacted, in dimethylformamide solution at 80° C., with one molar equivalent of a dialkyl vinyl phosphonate 29.9, (Aldrich), triethylamine and a catalytic amount of tetrakis(triphenylphosphine)palladium(0) to yield, after desilylation, the unsaturated phosphonate 29.10. The product is then reacted with diimide, prepared by basic hydrolysis of diethyl azodicarboxylate, as described in Angew. Chem. Int. Ed., 4, 271, 1965, to yield the saturated product 29.11.

Alternatively, 29.8 is reacted in toluene solution at ca. 100° C., with one molar equivalent of a dialkyl phosphite 29.2, triethylamine and 3 mol % tetrakis(triphenylphosphine)palladium(0), to give, after desilylation, the phosphonate product 29.12.

Using the above procedures, but employing, in place of the anhydride 29.7, different anhydrides 29.1, and/or different phosphonates 29.3, the corresponding products 29.4, 29.5 and 29.6 are obtained.

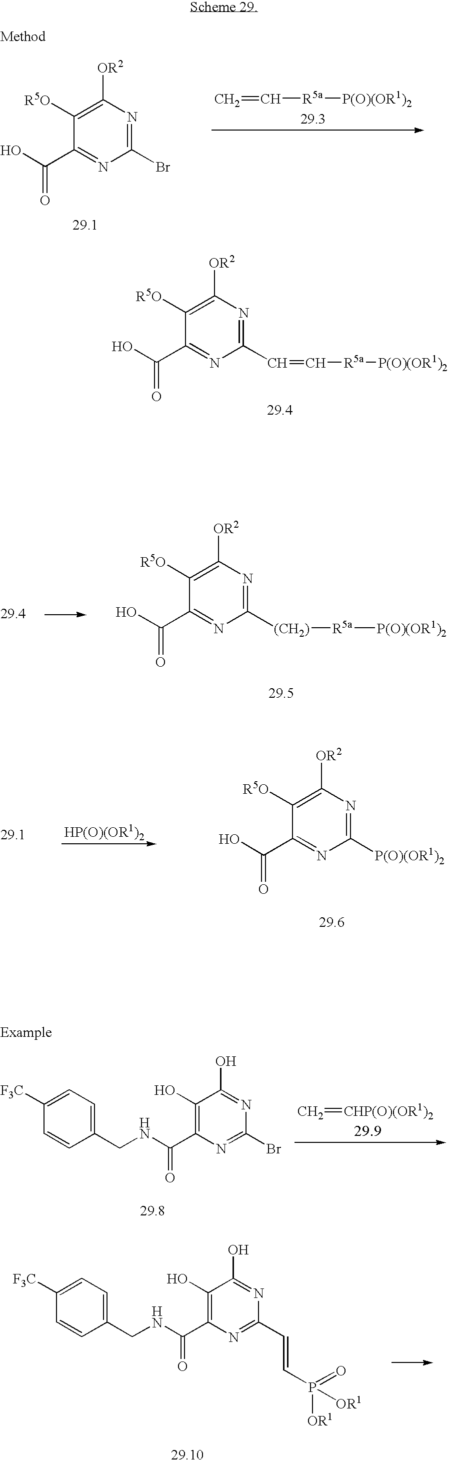

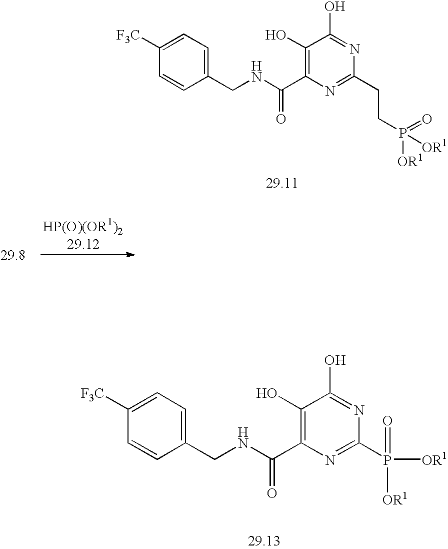

Scheme 30 illustrates the preparation of phosphonate esters Va in which the phosphonate is attached by means of a saturated or unsaturated carbon link at the 2-position. In this procedure, the amide 30.2 is condensed, under basic conditions, with a dialkyl formyl-substituted phosphonate 30.3, to afford the unsaturated phosphonate 30.4. The reaction is conducted at from ambient temperature to about 100° C., in a polar aprotic solvent such as dimethylformamide or dioxane, in the presence of a base such as sodium hydride, potassium tert. butoxide or lithium hexamethyldisilazide. Optionally, the product 30.4 is reduced, as described in Scheme 4, to afford the saturated analog 30.5.

For example, 3-(4-methoxy-benzyl)-2-methyl-4-oxo-5-triisopropylsilanyloxy-3,4-dihydro-pyrimidine-6-carboxylic acid (3,5-dichloro-benzyl)-ethyl-amide 30.7 is reacted, in dimethylformamide solution at 60° C., with one molar equivalent of a dialkyl formylmethyl phosphonate 30.8 (Aurora) and sodium hydride, to give, after desilylation, the unsaturated phosphonate 30.9. The product is then reacted with diimide, prepared by basic hydrolysis of diethyl azodicarboxylate, as described in Angew. Chem. Int. Ed., 4, 271, 1965, to yield the saturated phosphonate 30.10.

Using the above procedures, but employing, in place of the anhydride 30.6, different anhydrides 30.1, and/or different phosphonates 30.3, the corresponding products 30.4, and 30.5 are obtained.

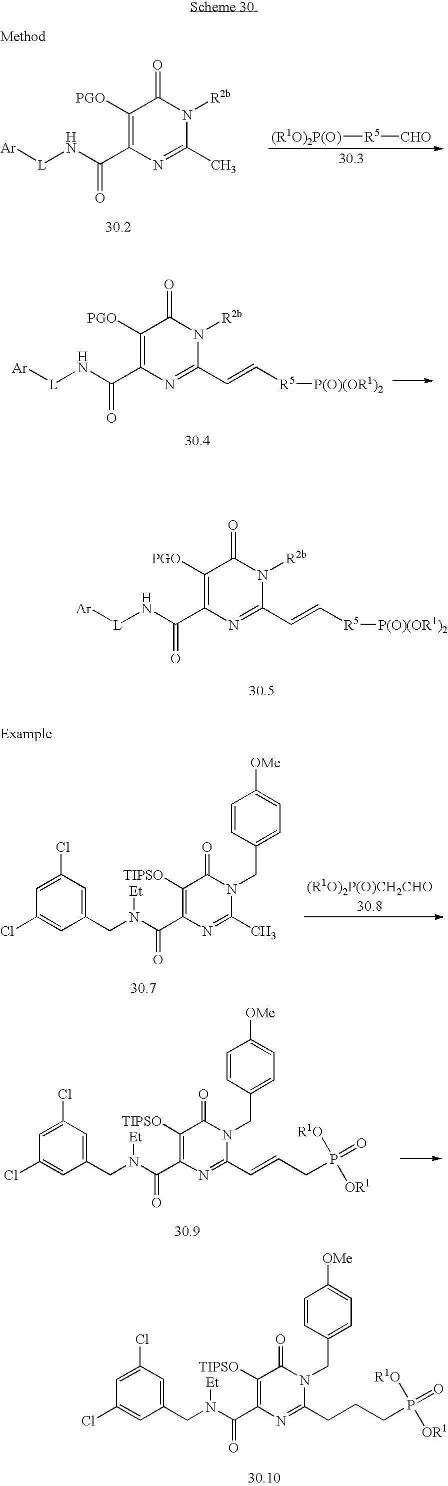

Scheme 31 illustrates the preparation of phosphonate esters IVa in which the phosphonate is attached by means of an oxime linkage at the 2-position. In this procedure, a 2-methyl, 6-amide 31.2 is brominated to give the 2-bromomethyl compound 31.3. Oxidation, as described in Scheme 26, of 31.3 affords the corresponding aldehyde 31.4. The aldehyde 31.4 is then converted, by reaction with hydroxylamine, into the oxime 31.5. The latter compound is then reacted, in a polar solvent such as tetrahydrofuran or dimethylformamide, in the presence of a base such as sodium hydroxide or potassium carbonate, with a dialkyl bromomethyl-substituted phosphonate 31.6, to prepare, after deprotection of the phenolic hydroxyl group, the oxime derivative 31.7.

For example, 2-formyl-4,5-dimethoxy-pyrimidine-6-carboxylic acid 4-fluoro-benzylamide 31.9 is reacted in tetrahydrofuran solution with three molar equivalents of hydroxylamine hydrochloride and sodium acetate, to produce 2-(hydroxyimino-methyl)-4,5-dimethoxy-pyrimidine-6-carboxylic acid 4-fluoro-benzylamide 31.10, which is then reacted in dioxane solution at ambient temperature, with one molar equivalent of a dialkyl bromopropyl phosphonate 31.11 (Synthelec) and potassium carbonate, to yield, after desilylation of the phenolic hydroxyl group, the oxime ether 31.12.

Also for example, a 2-phosphonate Formula IVa compound can be prepared with a morpholino linkage. The 5-hydroxyl of 3-[4-(4-Fluoro-benzylcarbamoyl)-5-hydroxy-3-methyl-4-oxo-3,4-dihydro-pyrimidin-2-yl]-morpholine-4-carboxylic acid tert-butyl ester 31.13 can be esterified as the 2-iodobenzoate to give 31.14. The Boc group can be removed under acidic conditions from 31.14 and the amino group of 2-iodo-benzoic acid 4-(4-fluoro-benzylcarbamoyl)-1-methyl-2-morpholin-3-yl-6-oxo-1,6-dihydro-pyrimidin-5-yl ester 31.15 may be condensed with aldehyde 31.16 to give 31.17 by reductive amination with sodium cyanoborohydride. The 2-iodobenzoate group may be removed under mild oxidative conditions, following the methods of R. Moss et al, Tetrahedron Letters, 28, 5005 (1989), to give morpholino phosphonate 31.18.

Using the above procedures, but employing, in place of the anhydride 31.8, different anhydrides 31.1, and/or different phosphonates 31.6, the corresponding products 31.7 are obtained.

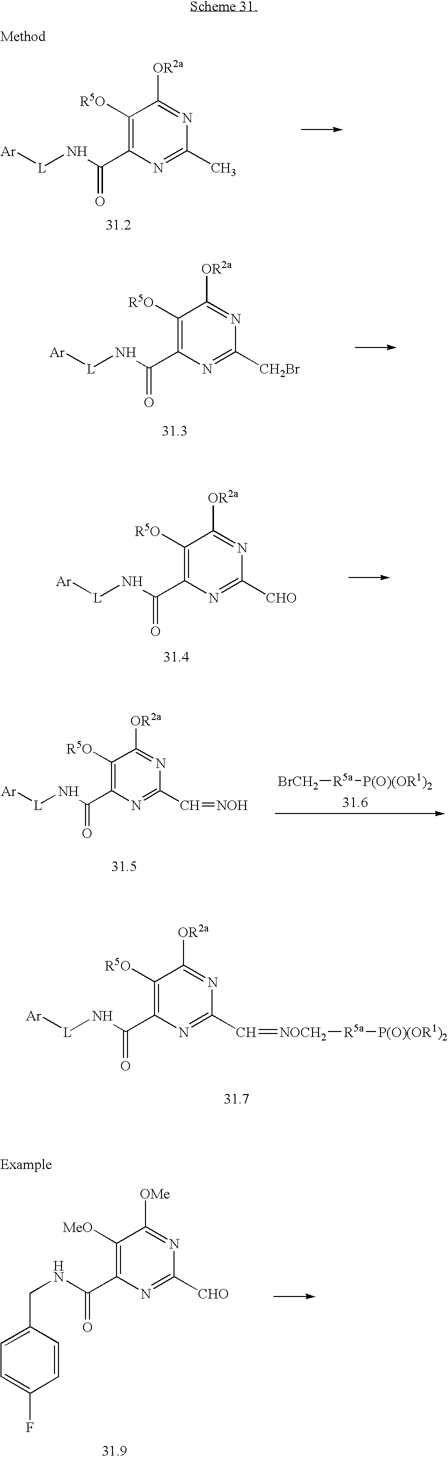

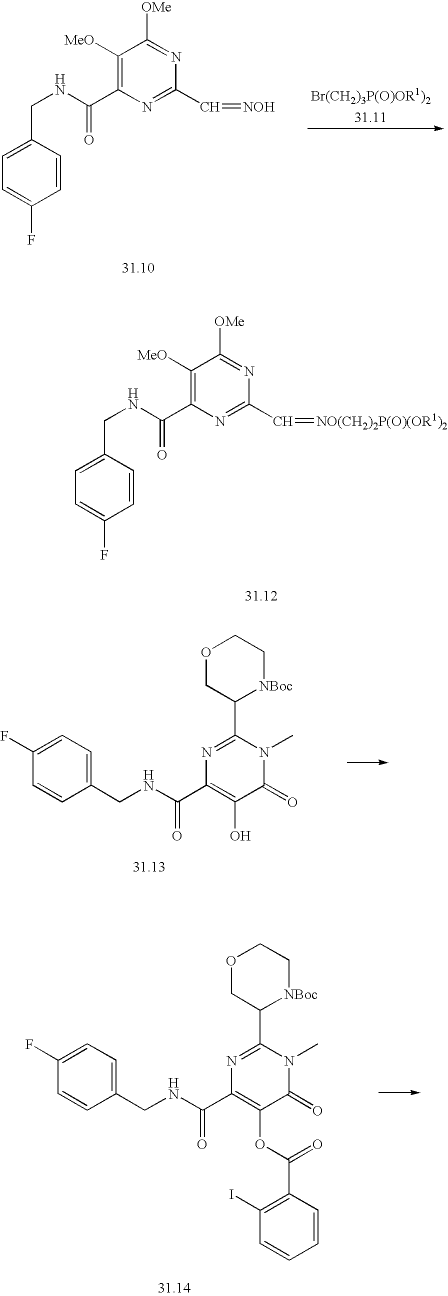

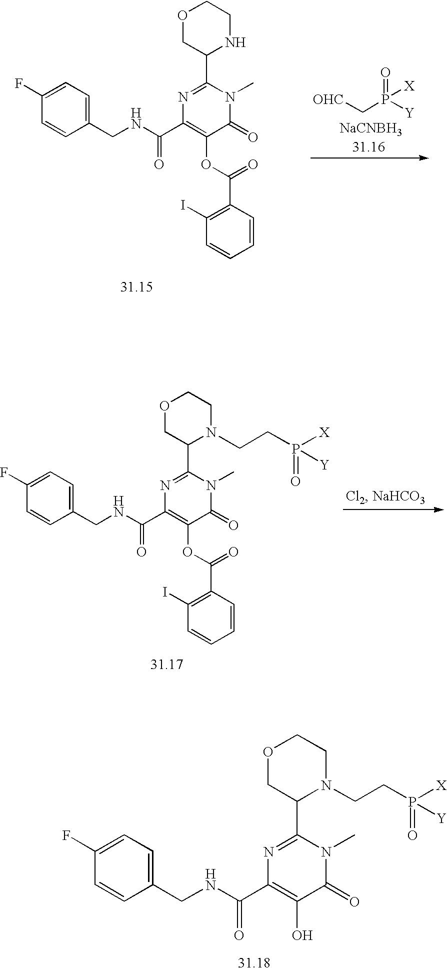

The preparation of phosphonate esters and the interconversion of such esters to other phosphonate analogs of the invention can be carried out as described in WO 2004/096237 A2 pages 110-144.

### EXAMPLES GENERAL SECTION

Some Examples have been performed multiple times. In repeated Examples, reaction conditions such as time, temperature, concentration, and the like, and yields were within normal experimental ranges. In repeated Examples where significant modifications were made, these have been noted where the results varied significantly from those described. In Examples where different starting materials were used, these are noted. When the repeated Examples refer to a “corresponding” analog of a compound, such as a “corresponding ethyl ester”, this intends that an otherwise present group, in this case typically a methyl ester, is taken to be the same group modified as indicated.

Synthesis of HIV-Integrase Inhibitor Compounds

### EXAMPLES

### Example 1

### N-4-fluorobenzyl-succinimide 1

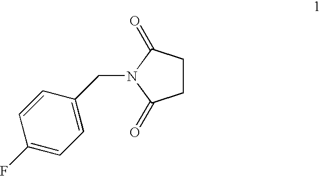

Freshly ground potassium carbonate, K₂CO₃ (31 g, 225 mmol) was added to dry acetone (200 ml) in a 3-necked flask equipped with drying tube, condenser, and mechanical stirrer. Succinimide (7.43 g, 75 mmol) and 4-fluorobenzylbromide (11.21 mL, 90 mmol) were added. The mixture was refluxed for 19 hours and filtered through Celite. Acetone was removed under vacuum, diluted with EtOAc, washed with saturated aqueous sodium bicarbonate and also with brine, dried (MgSO₄), filtered and concentrated to give crude. Crude product was chromatographed (EtOAc/Hexane) on silica gel to give N-4-fluorobenzyl-succinimide 1 as white solid (13.22 g, 85%). ¹H NMR (CDCl₃) δ 7.4 (dd, 2H), 7.0 (t, 2H), 4.6 (s, 1H), 2.7 (s, 4H).

### Example 2

### 5,8-Dihydroxy-[6,7]-N-(4-fluorobenzyl)-succinimido-quinoline 2

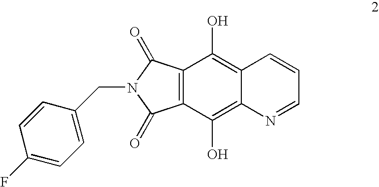

N-4-fluorobenzyl-succinimide 1 (8 g, 38.6 mmol) and 2,3-pyridine carboxylic acid dimethyl ester (7.9 g, 40.6 mmol) were dissolved in dry tetrahydrofuran (THF, 78 mL) and dry methanol (MeOH, 1.17 mL) in a 3-necked flask with mechanical stirrer and condenser. Sodium hydride (NaH, 60% in mineral oil, 3.4 g, 85 mmol) was added slowly in four portions. The mixture was stirred until bubbling ceased, then refluxed for 24 hours. HCl (30 mL 6 M) was then added to the mixture while in an ice bath, with stirring for 15 minutes. Diethylether (100 mL) was added. The precipitate was filtered, washed with diethylether and H₂O, and dried under vacuum at 100° C. Crude product was then recrystallized from 1 L refluxing dioxane and dried under vacuum at 100° C. to give solid 5,8-Dihydroxy-[6,7]-N-(4-fluorobenzyl)-succinimido-quinoline 2 (8.6 g, 66%). ¹H NMR (CD₃SOCD₃) δ 9.05 (d, 1H), 8.75 (d, 1H), 7.79 (dd, 1H), 7.37 (dd, 2H), 7.17 (t, 2H), 4.73 (s, 2H). mp: 281.9-284.0.

### Example 3

### 5-O-Propanoate, 8-hydroxy-[6,7]-N-(4-fluorobenzyl)-succinimido-quinoline 3

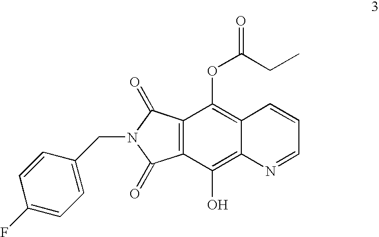

5,8-Dihydroxy-[6,7]-N-(4-fluorobenzyl)-succinimido-quinoline 2 is acylated with propanoyl chloride to give 5-O-propanoate, 8-hydroxy-[6,7]-N-(4-fluorobenzyl)-succinimido-quinoline 3.

### Example 4

### Carbonic acid ethyl ester 7-(4-fluoro-benzyl)-9-hydroxy-6,8-dioxo-7,8-dihydro-6H-pyrrolo[3,4-g]quinolin-5-yl ester 4

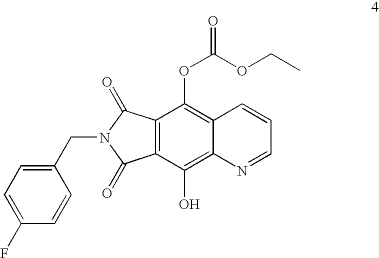

5,8-Dihydroxy-[6,7]-N-(4-fluorobenzyl)-succinimido-quinoline (300 mg, 0.887 mmol) 2 was suspended in 1,4 dioxane (5 mL) and water (20 mL). An aqueous solution of NaOH (0.567 M, 3.1 mL) was added slowly to form red solution which was then cooled in an ice-water bath. Ethyl chloroformate (0.093 mL, 0.975 mmol) was added and the mixture was stirred at room temperature for 30 minutes. Dichloromethane and 1N aqueous HCl were added to the mixture in a separate. The aqueous layer was extracted with dichloromethane two more times. The combined organic solution was washed with brine, dried (MgSO4) and concentrated. The crude product was crystallized from EtOAc to give carbonic acid ethyl ester 7-(4-fluoro-benzyl)-9-hydroxy-6,8-dioxo-7,8-dihydro-6H-pyrrolo[3,4-g]quinolin-5-yl ester 4 (136 mg, 37%) as a yellow solid. ¹H NMR (CDCl₃) δ 9.0 (d, 1H), 8.5 (d, 1H), 7.7 (dd, 1H), 7.5 (t, 2H), 7.4 (t, 2H), 7.0 (t, 2H), 4.8 (s, 2H), 4.5 (q, 2H), 1.5 (t, 3H); MS: 409 (M−1)

### Example 5

### Carbonic acid ethyl ester 7-(4-fluoro-benzyl)-9-methoxymethoxy-6,8-dioxo-7,8-dihydro-6H-pyrrolo[3,4-g]quinolin-5-yl ester 5

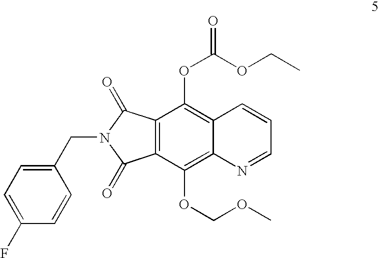

Carbonate (23.6 mg, 0.08 mmol) 4 was dissolved in acetonitrile (2 mL). Chloromethyl methyl ether (0.013 mL, 0.17 mmol) and Cs₂CO₃ (74 mg, 0.23 mmol) were added consecutively. The mixture was stirred at room temperature for 30 minutes when most of the starting material was consumed as indicated by TLC. Dichloromethane was added and the solution was washed with 1N HCl and brine, dried (MgSO₄) and concentrated. The crude product was chromatographed on silica gel column, eluting with EtOAc/hexanes to give the product, carbonic acid ethyl ester 7-(4-fluoro-benzyl)-9-methoxymethoxy-6,8-dioxo-7,8-dihydro-6H-pyrrolo[3,4-g]quinolin-5-yl ester 5 as a white solid (18 mg, 70%). ¹H NMR (CDCl₃) δ 9.1 (dd, 1H), 8.5 (dd, 1H), 7.7 (dd, 1H), 7.4 (dd, 2H), 7.0 (t, 2H), 5.9 (s, 2H), 4.8 (s, 2H), 4.5 (q, 2H), 3.7 (s, 1H), 1.5 (t, 3H).

### Example 6

### 7-(4-Fluoro-benzyl)-5-hydroxy-9-methoxymethoxy-pyrrolo[3,4-g]quinoline-6,8-dione 6

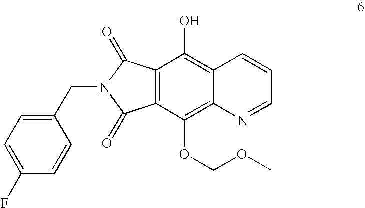

To the ethyl carbonate methoxymethyl ether 5 (70.9 mg, 0.156 mmol) in THF (7.6 mL) at room temperature was added a solution (5 mL) of K₂CO₃ (215 mg, 1.56 mmol) in water and 4-dimethylaminopyridine (3.8 mg, 0.03 mmol). The yellow solution was stirred at room temperature under nitrogen atmosphere overnight. Most of THF was removed under reduced pressure at 30-40° C. and the remaining solution was diluted with dichloromethane, washed with 1N HCl and brine, dried (MgSO₄) and concentrated to give solid crude product (51 mg, 85%), which is triturated in diethylether/hexane to afford the product, 7-(4-fluoro-benzyl)-5-hydroxy-9-methoxymethoxy-pyrrolo[3,4-g]quinoline-6,8-dione 6 as a yellow solid (34 mg). ¹H NMR (CDCl₃) δ 9.1 (dd, 1H), 8.7 (dd, 1H), 7.6 (dd, 1H), 7.4 (dd, 2H), 7.0 (t, 2H), 5.8 (s, 2H), 4.8 (s, 2H), 3.7 (s, 1H). MS: 383 (M+1); 381 (M−1).

### Example 7

### Trifluoro-methanesulfonic acid 7-(4-fluoro-benzyl)-9-methoxymethoxy-6,8-dioxo-7,8-dihydro-6H-pyrrolo[3,4-g]quinolin-5-yl ester 7

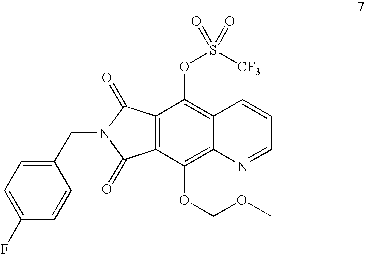

To the methoxymethyl ether 6 (13.7 mg, 0.036 mmol) in dichloromethane (1 mL) at −78° C. were added N,N-diisopropylethylamine (0.019 mL, 0.1 mmol) and trifluoromethanesulfonic anhydride (0.012 mL, 0.054 mmol) successively. The solution was stirred at the same temperature for 30 minutes and diluted with dichloromethane, washed with water and brine, dried (MgSO₄) and concentrated, The mixture was chromatographed on a silica gel column, eluting with EtOAc/hexanes to afford the product, trifluoro-methanesulfonic acid 7-(4-fluoro-benzyl)-9-methoxymethoxy-6,8-dioxo-7,8-dihydro-6H-pyrrolo[3,4-g]quinolin-5-yl ester 7 (6 mg, 33%). ¹H NMR (CDCl₃) δ 9.1 (dd, 1H), 8.5 (dd, 1H), 7.8 (dd, 1H), 7.5 (dd, 2H), 7.0 (t, 2H), 5.9 (s, 2H), 4.9 (s, 2H), 3.7 (s, 1H). ¹⁹F NMR (CDCl₃) δ −72.8.

The reaction was repeated, where monophenol 6 (0.0444 g, 0.116 mmol) was dissolved in 2 mL dry dichloromethane. To this was added diisopropylethylamine (0.06 mL, 0.348 mmol.) After cooling to −78° C., triflic anhydride was added (0.029 mL, 0.342 mmol) and was stirred at this temperature for thirty minutes. Reaction was then complete by TLC, diluted with dichloromethane, washed with 1M HCl, saturated NaHCO₃ solution, dried (MgSO₄) and organics concentrated to give product, trifluoro-methanesulfonic acid 7-(4-fluoro-benzyl)-9-methoxymethoxy-6,8-dioxo-7,8-dihydro-6H-pyrrolo[3,4-g]quinolin-5-yl ester 7 (0.06 g, 0.116 mmol, 100%) which was used as crude for the next reaction. ¹H NMR (CDCl₃) δ 9.15 (dd, 1H), 8.46 (d, 1H), 7.47 (dd, 1H), 7.01 (t, 2H), 5.92 (s, 2H), 4.87 (s, 2H.), 3.67 (s, 3H); MS: 537 (M+Na).

### Example 8

### 7-(4-Fluoro-benzyl)-5-methoxy-9-methoxymethoxy-pyrrolo[3,4-g]quinoline-6,8-dione 8

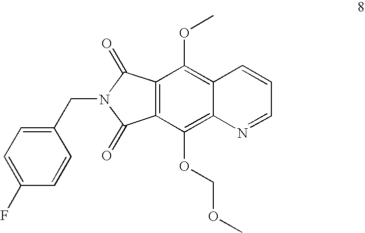

Methoxymethyl ether 6 (0.02 g, 0.052 mmol) was dissolved in 2 mL dry dichloromethane at 0° C. An excess of a diazomethane solution in diethylether was added. After about 20 minutes, all starting 6 was consumed. The mixture was concentrated in vacuo to give crude 7-(4-fluoro-benzyl)-5-methoxy-9-methoxymethoxy-pyrrolo[3,4-g]quinoline-6,8-dione 8 (0.0223 g, 0.0527 mmol). ¹H NMR (CDCl₃) δ 9.1 (dd, 1H), 8.7 (dd, 1H), 7.6 (dd, 1H), 7.5 (t, 2H), 7.0 (t, 2H), 5.8 (s, 2H), 4.8 (s, 2H), 4.4 (s, 3H), 3.7 (s, 3H). MS: 397 (M+1); 419 (M+23).

### Example 9

### 7-(4-Fluoro-benzyl)-9-hydroxy-5-methoxy-pyrrolo[3,4-g]quinoline-6,8-dione 9

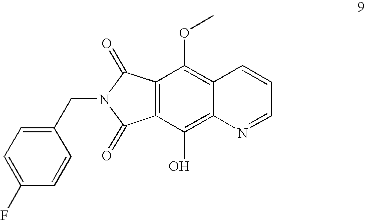

Crude diether 8 (0.0223 g, 0.0527 mmol) was dissolved in 1 mL dichloromethane. Ten equivalents of trifluoroacetic acid was added. the mixture was stirred at room temperature for 45 minutes. The reaction mixture was concentrated and azeotroped with toluene (2×) to give crude 7-(4-fluoro-benzyl)-9-hydroxy-5-methoxy-pyrrolo[3,4-g]quinoline-6,8-dione 9 which was triturated with 8 mL of 1:1 diethylether/hexane and filtered to give 9 (0.0161 g, 0.0456 mmol, 83% for two steps). ¹H NMR (CDCl₃) δ 9.0 (br s, 1H), 8.7 (d, 1H), 7.7 (d, 1H), 7.5 (m, 2H), 7.0 (t, 2H), 4.8 (s, 2H), 4.4 (s, 3H). MS: 353 (M+1).

### Example 10

### 5-Allyloxy-7-(4-fluoro-benzyl)-9-methoxymethoxy-pyrrolo[3,4-g]quinoline-6,8-dione 10

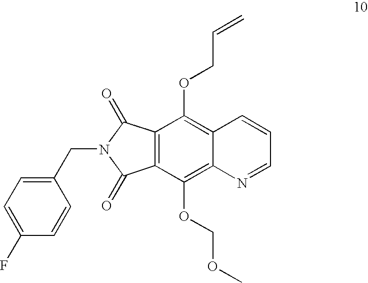

Methoxymethyl ether 6 (0.0172 g, 0.045 mmol) was dissolved in 1.5 mL dry dimethylformamide (DMF). Ground K₂CO₃ (0.0186 g, 0.135 mmol) was added, followed by allyl bromide (0.0077 mL, 0.09 mmol). The mixture was stirred at room temperature overnight, then diluted with 100 mL of ethylacetate, washed with saturated NH₄Cl solution, dried (MgSO₄), and concentrated to give crude 10. The crude product 10 was chromatographed on silica gel, eluting with ethylacetate and hexanes to give white solid allyl, methoxymethyl diether 10: (0.0063 g, 33%). ¹H NMR (CDCl₃) δ 9.1 (dd, 1H), 8.8 (dd, 1H), 7.6 (dd, 1H), 7.5 (dd, 2H), 7.0 (t, 2H), 6.1 (m, 1H), 5.8 (s, 2H), 5.5 (d, 1H), 5.3 (d, 1H), 5.1 (d, 2H), 4.8 (s, 2H). MS: 423 (M+1); 445 (M+23).

### Example 11

### 5-Allyloxy-7-(4-fluoro-benzyl)-9-hydroxy-pyrrolo[3,4-g]quinoline-6,8-dione 11

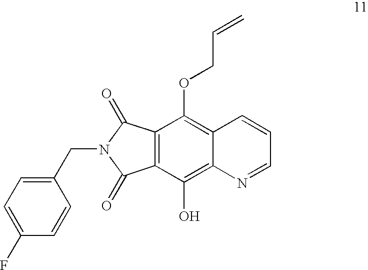

5-Allyloxy-7-(4-fluoro-benzyl)-9-methoxymethoxy-pyrrolo[3,4-g]quinoline-6,8-dione 10 was dissolved in 1 mL dichloromethane. Ten equivalents of trifluoroacetic acid was added and the mixture was stirred at room temperature. After one hour another 10 equivalents of trifluoroacetic acid was added. The mixture was then stirred overnight, concentrated in vacuo, and azeotroped with toluene (2×), to give crude 11 which was triturated with 2 mL of 1:1 diethylether/hexane two times to give allyl ether 11 (0.0025 g, 0.0066 mmol, 44%). ¹H NMR (CDCl₃) δ 9.0 (s, 1H), 8.7 (d, 1H), 7.7 (m, 1H), 7.5 (m, 2H), 7.0 (t, 2H), 6.1 (m, 1H), 5.4 (d, 1H), 5.3 (d, 1H), 5.1 (d, 2H), 4.8 (s, 2H). MS: 379 (M+1).

### Example 12

### 7-(4-Fluoro-benzyl)-5-hydroxy-9-triisopropylsilanyloxy-pyrrolo[3,4-g]quinoline-6,8-dione 12

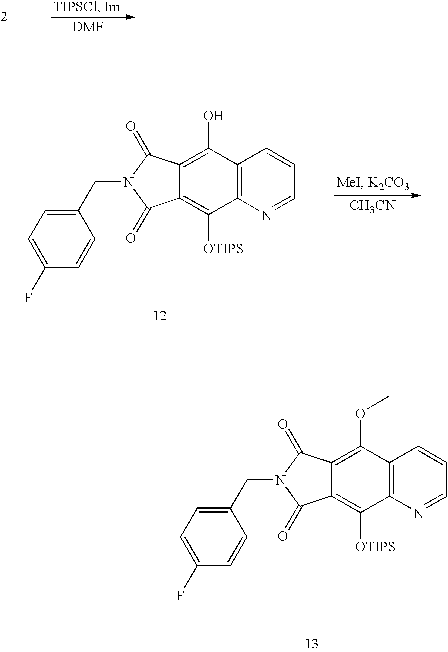

A solution of 7-(4-fluoro-benzyl)-5,9-dihydroxy-pyrrolo[3,4-g]quinoline-6,8-dione 2 (1.039 g, 3.07 mmol) in 31 mL of DMF was stirred with imidazole (314 mg, 4.62 mmol) and triisopropylsilylchloride (TIPSCl, 0.723 mL, 3.38 mmol) under a N₂ atmosphere for 1.5 days when most of the starting materials was converted to the regiospecific mono TIPS (triisopropylsilyl) protected compound. The solid bisphenol left in the reaction was filtered and recycled. The mother liquor was dried and the residue was suspended in EtOAc. The organic layer was washed with water and dried. The resulted solid 12 was carried to the next step. EI MS (m/z) 495.6 [MH⁺], 517.4 [M+Na].

### Example 13

### 7-(4-Fluoro-benzyl)-5-methoxy-9-triisopropylsilanyloxy-pyrrolo[3,4-g]quinoline-6,8-dione 13

A mixture of 12 from the monosilylation reaction was heated at 40° C. in anhydrous acetonitrile with K₂CO₃ (1.64 g, 11.8 mmol) and methyl iodide (4.2 g, 29.6 mmol) for 5 hours. The reaction mixture was worked up by addition of H₂O and EtOAc. The organic layer was washed with H₂O and the solvent was removed in vacuo. The residue was purified by column chromatography using a gradient of 10% EtOAc-Hex to elute the product 13 as a yellow solid (72% for two steps). ¹H NMR (300 MHz, CDCl₃) δ 1.13 (d, 18H, J=8 Hz), 1.53 (septet, 3H, J=7 Hz), 4.29 (s, 3H), 4.84 (s, 2H), 7.00 (t, 2H, J=8 Hz), 7.48 (dd, 2H, J=5, 8 Hz), 7.58 (dd, 1H, J=4, 8 Hz), 8.65 (dd, 1H, J=2, 8 Hz), 8.93 (dd, 1H, J=2, 4 Hz); EI MS (m/z) 509.7 [MH⁺], 531.4 [M+Na].

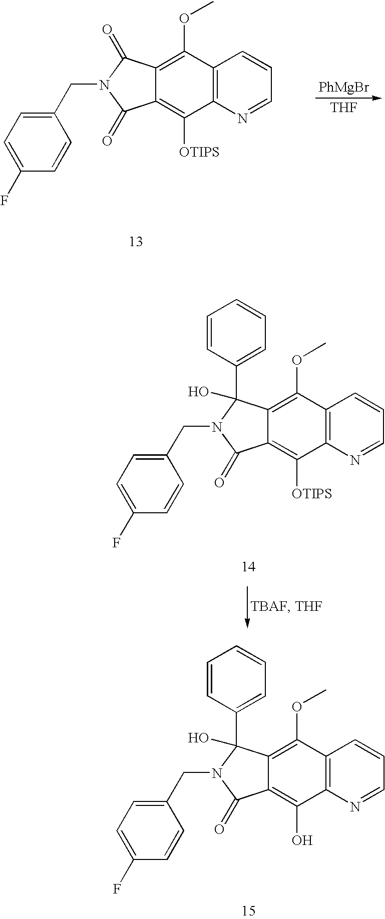

### Example 14

### 7-(4-Fluoro-benzyl)-6-hydroxy-5-methoxy-6-phenyl-9-triisopropylsilanyloxy-6,7-dihydro-pyrrolo[3,4-g]quinolin-8-one 14

A mixture of 13 (36 mg, 0.071 mmol) in 0.35 mL of dry THF was cooled to 0° C. A 26 μL aliquot of a 3 M solution of phenyl magnesium bromide in ether (0.078 mmol) was added to the mixture and the reaction was allowed to warn up to room temperature. The reaction was worked up in 30 minutes when the reaction was complete as indicated by TLC. The mixture was diluted with EtOAc and washed with water. The product 14 was purified by column chromatography using 20% EtOAc-Hex solvent system to provide 33 mg (80%) of the product as a solid. ¹H NMR (300 MHz, CDCl₃) δ 1.20 (s, 18H), 1.52-1.68 (m, 3H), 2.95 (s, 1H), 3.93 (s, 3H), 4.08 (d, 1H, J=15 Hz), 4.77 (d, 1H, J=15 Hz), 6.85 (t, 2H, J=9 Hz), 7.19-7.25 (m, 2H), 7.25-7.35 (m, 3H), 7.39-7.49 (m, 3H), 8.26 (d, 1H, J=8 Hz), 8.84 (br d, 1H, J=4 Hz); ¹⁹F NMR (282.6 MHz, CDCl₃) δ −76.2, 60.7; EI MS (m/z) 587.5 [MH⁺], 609.4 [M+Na].

### Example 15

### 7-(4-Fluoro-benzyl)-6,9-dihydroxy-5-methoxy-6-phenyl-6,7-dihydro-pyrrolo[3,4-g]quinolin-8-one 15

A mixture of 14 (27 mg, 0.046 mmol) in THF (0.46 mL) and tetrabutyl ammonium fluoride (50 μL, 0.050 mmol) was stirred at room temperature under a N₂ atmosphere for 2 hours when reaction was complete as demonstrated by LCMS analysis. The organic solvent was removed in vacuo and the residue was suspended in EtOAc. The organic layer was washed with water and dried. The solid was washed with hexane and dried to provide 15 mg (76%) of the product 15 as a light orange solid. ¹H NMR (300 MHz, CD₃OD) δ 3.54 (s, 3H), 4.36 (d, 1H, J=15 Hz), 4.48 (d, 1H, J=15 Hz), 6.84 (t, 2H, J=9 Hz), 7.17-7.23 (m, 2H), 7.24-7.26 (m, 3H), 7.35-7.46 (m, 2H), 7.62 (dd, 1H, J=4, 9 Hz), 8.44 (d, 1H, J=9 Hz), 8.89 (d, 1H, J=3 Hz); ¹⁹F NMR (282.6 MHz, CDCl₃) δ 58.5; EI MS (m/z) 431.2 [MH⁺], 453.2 [M+Na].

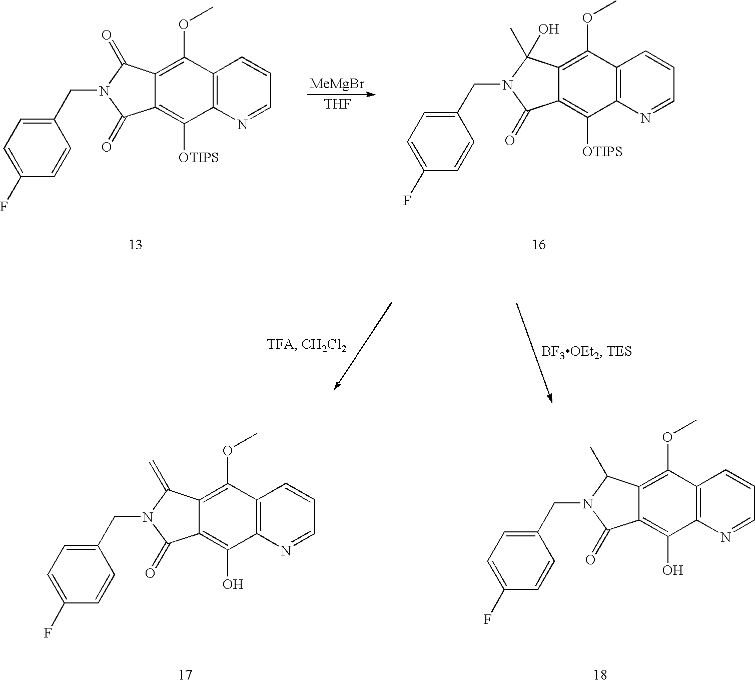

### Example 16

### 7-(4-Fluoro-benzyl)-6-hydroxy-5-methoxy-6-methyl-9-triisopropylsilanyloxy-6,7-dihydro-pyrrolo[3,4-g]quinolin-8-one 16

Under a nitrogen atmosphere, a solution of 13 (90 mg, 0.18 mmol) was dissolved in 0.885 mL of dry THF. A solution of 3 M of methylmagnesium bromide in ether (71 μL, 0.213 mmol) was added. The solution was allowed to stir at ambient temperature for 2 hours when TLC indicated complete consumption of starting materials. The reaction mixture was diluted with EtOAc and washed with water and saturated aqueous NH₄Cl. The organic layer was reduced in vacuo to 1 mL and cooled to get the product 16 to crystallize from the solvent (92 mg, 99%). ¹H NMR (300 MHz, CDCl₃) δ 1.16 (d, 18H, J=8 Hz), 1.55 (septet, 3H, J=8 Hz), 1.78 (s, 3H), 2.29 (s, 1H), 4.04 (s, 3H), 4.72 (ABqt, 2H, J=13 Hz), 6.99 (t, 2H, J=9 Hz), 7.38 (dd, 2H, J=6, 9 Hz), 7.52 (dd, 1H, J=4, 9 Hz), 8.42 (dd, 1H, J=2, 8 Hz), 8.87 (dd, 1H, J=2, 4 Hz); ¹⁹F NMR (282.6 MHz, CDCl₃) δ 60.8.

### Example 17

### 7-(4-Fluoro-benzyl)-9-hydroxy-5-methoxy-6-methylene-6,7-dihydro-pyrrolo[3,4-g]quinolin-8-one 17

A solution of 16 (10 mg, 0.019 mmol) in 3 mL of CH₂Cl₂ and TFA (30 μL, 0.389 mmol) was aged for 18 hours. Analysis of the reaction demonstrated complete conversion of starting materials to the product. The solvents were removed under reduced pressure. The residue was dissolved in EtOAc and precipitated with hexanes. The mother liquor was removed and the solid residue was washed with hexanes and subsequently with Et₂O to yield the product 17 as a solid. ¹H NMR (300 MHz, CDCl₃) δ 3.97 (s, 3H), 4.99 (s, 2H), 5.04 (d, 1H, J=2 Hz), 5.63 (d, 1H, J=2 Hz), 6.90 (br s, 1H), 7.04 (t, 2H, J=8 Hz), 7.31 (dd, 2H, J=5, 8 Hz), 7.71 (dd, 1H, J=4, 8 Hz), 8.64 (dd, 1H, J=2, 9 Hz), 9.11 (d, 1H, J=3 Hz); ¹⁹F NMR (282.6 MHz, CDCl₃) δ 62.1; EI MS (m/z) 351.5 [MH⁺], 383.3 [M+Na].

### Example 18

### 7-(4-Fluoro-benzyl)-9-hydroxy-5-methoxy-6-methyl-6,7-dihydro-pyrrolo[3,4-g]quinolin-8-one 18

To a solution of 16 (52 mg, 0.099 mmol) in 1.4 mL of dry CH₂Cl₂ under a N₂ atmosphere, was added BF₃.OEt₂ (49 μL, 0.397 mmol) followed by triethylsilane (63 μL, 0.397 mmol). The solution was allowed to stir at ambient temperature for 1 day when LCMS indicated a clean conversion of starting materials to the desired product. The reaction was worked up by removing the solvent and dissolving the residue in EtOAc. The organic layer was washed with water and the solvent removed under reduced pressure. The residue was dissolved in 1 mL of EtOAc and triturated by addition of hexanes to provide the product 18. ¹H NMR (300 MHz, CDCl₃) δ 1.60 (d, 3H, J=7 Hz), 3.93 (s, 3H), 4.28 (d, 1H, J=15 Hz), 4.65 (q, 1H, J=7 Hz), 5.25 (d, 1H, J=15 Hz), 7.06 (t, 2H, J=8 Hz), 7.32 (dd, 2H, J=6, 8 Hz), 7.67 (dd, 1H, J=4, 8 Hz), 8.59 (br s, 1H), 8.61 (d, 1H, J=8 Hz), 9.11 (br s, 1H); ¹³C NMR (75 MHz, CDCl₃) δ 16.9, 42.8, 54.5, 61.9, 113.9, 115.7, 116.0, 122.7, 126.6, 129.8, 129.9, 130.8, 132.1, 133.1, 136.7, 142.4, 147.8, 148.3, 162.3 (d, J=245 Hz), 168.1; ¹⁹F NMR (282.6 MHz, CDCl₃) δ 62.5; EI MS (m/z) 353.5 [MH⁺], 385.4 [M+Na].

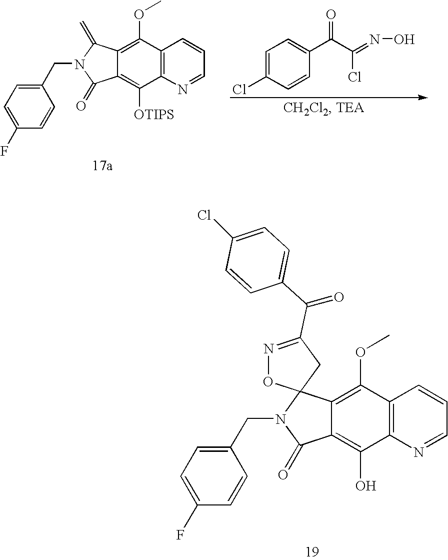

### Example 19

### Isoxazole 19

The exocyclic olefin in 17 can be utilized toward a cycloaddition reaction. Under a nitrogen atmosphere, a TIPS protected analog 17a (17 mg, 0.033 mmol) was suspended in 0.17 mL of dry CH₂Cl₂. To this solution was added 4-chlorophenylglyoxyl-O-hydroxamyl chloride (7.3 mg, 0.034 mmol) and TEA (4.7 μL, 0.034 mmol). The solution was stirred at room temperature for 12 hours. The reaction was worked up by diluting the solution with EtOAc and washing the organic layer with water. The organic layer was removed under reduced pressure. The residue was dissolved in EtOAc and diluted with hexanes. The solution was filtered and the mother liquor was dried to provide 18 mg (100%) of the product 19 as a white solid. ¹H NMR (300 MHz, CDCl₃) δ 3.31 (d, 1H, J=19 Hz), 3.94 (s, 3H), 4.01 (d, 1H, J=19 Hz), 4.36 (d, 1H, J=16 Hz), 4.96 (d, 1H, J=15 Hz), 6.95 (t, 2H, J=9 Hz), 7.29 (dd, 2H, J=5, 9 Hz), 7.55 (d, 2H, J=9 Hz), 7.65 (dd, 1H, J=4, 8 Hz), 8.29 (d, 2H, J=9 Hz), 8.45 (dd, 1H, J=2, 9 Hz), 8.99 (dd, 1H, J=2, 4 Hz); ¹⁹F NMR (282.6 MHz, CDCl₃) δ 62.8; EI MS (m/z) 532.6 [MH⁺].

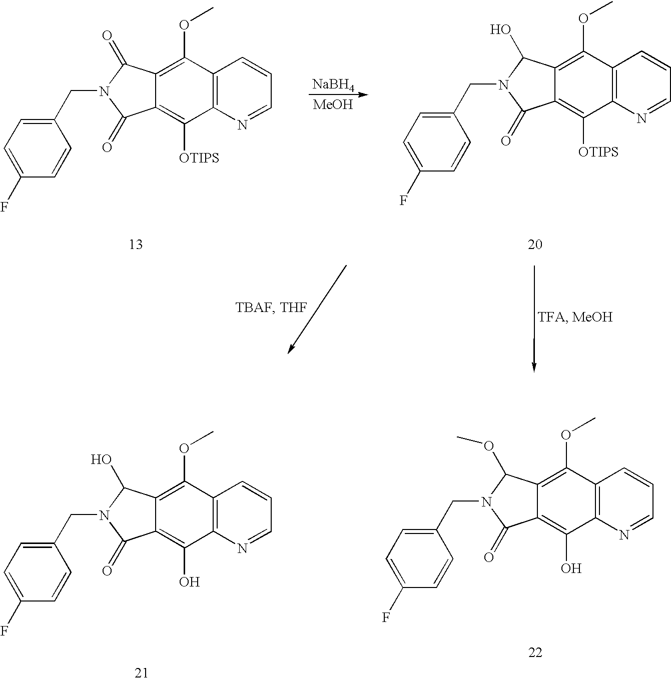

### Example 20

### 7-(4-Fluoro-benzyl)-6,9-dihydroxy-5-methoxy-6,7-dihydro-pyrrolo[3,4-g]quinolin-8-one 20

To a solution of 13 (0.699 g, 1.38 mmol) in 14 mL of a 1:1 solution of dry MeOH:CH₂Cl₂ under a N₂ atmosphere was added sodium borohydride (NaBH₄, 156 mg, 4.13 mmol). The reaction mixture was dried after 5 hours and the residue was loaded onto a silica column. The product was eluted with a 10% EtOAc-Hex to provide the product 20. ¹H NMR (300 MHz, CDCl₃) δ 1.10 (d, 9H, J=8 Hz), 1.16 (d, 9H, J=7 Hz), 1.52 (septet, 3H, J=8 Hz), 3.72 (d, 1H, J=11 Hz), 4.11 (s, 3H), 4.23 (d, 1H, J=15 Hz), 4.85 (d, 1H, J=15 Hz), 5.79 (d, 1H, J=11 Hz), 6.97 (t, 2H, J=9 Hz), 7.27 (dd, 2H, J=6, 9 Hz), 7.43 (dd, 1H, J=4, 8 Hz), 8.43 (dd, 1H, J=2, 8 Hz), 8.81 (dd, 1H, J=2, 4 Hz); ¹³C NMR (75 MHz, CDCl₃) δ 14.8. 18.2, 41.3, 61.6, 78.6, 115.3, 115.6, 116.6, 122.3, 126.0, 126.8, 130.1, 130.2, 131.1, 132.8, 143.1, 143.8, 148.3, 162.1 (d, J=244 Hz), 165.2; EI MS (m/z) 511.5 [MH⁺], 533.4 [M+Na].

### Example 21

### 7-(4-Fluoro-benzyl)-6,9-dihydroxy-5-methoxy-6,7-dihydro-pyrrolo[3,4-g]quinolin-8-one 21

A solution of 20 (35 mg, 0.069 mmol) was stirred in 0.69 mL of dry THF and 75 μL of a 1 M solution of tetra-butylammonium fluoride (TBAF, 0.075 mmol) under a N₂ atmosphere for 2 hours at ambient temperature. The solution was diluted with EtOAc and the organic layer was washed with water. The organic layer was removed in vacuo to leave a yellow residue. The solid was washed with hexanes and dried to give 27 mg (100%) of the product 21. ¹H NMR (300 MHz, CD₃OD) δ4.13 (s, 3H), 4.46 (d, 1H, J=15 Hz), 5.04 (d, 1H, J=15 Hz), 6.01 (s, 1H), 7.09 (t, 2H, J=9 Hz), 7.42-7.47 (m, 2H), 7.65 (dd, 1H, J=4, 9 Hz), 8.61 (d, 1H, J=8 Hz), 8.89 (d, 1H, J=3 Hz); ¹³C NMR (75 MHz, CD₃OD) δ 41.1, 79.3, 60.0, 111.6, 115.0, 115.4, 122.4, 125.1, 125.9, 129.6, 130.0, 131.5, 132.9, 139.5, 142.8, 148.8, 161.8 (d, J=245 Hz), 166.7; ¹⁹F NMR (282.6 MHz, CDCl₃) δ 59.4; EI MS (m/z) 355.4 [MH⁺].

### Example 22

### 7-(4-Fluoro-benzyl)-9-hydroxy-5,6-dimethoxy-6,7-dihydro-pyrrolo[3,4-g]quinolin-8-one 22

A solution of 21 (6.7 mg, 0.019 mmol) in a 1:1 solution of CH₂Cl₂:MeOH was stirred with TFA (3 μL, 0.038 mmol) at room temperature for 2 hours when complete conversion was observed by LCMS. The solution was dried in vacuo and the residue was washed with hexanes to yield 7 mg of the product 22. EI MS (m/z) 355.4 [MH⁺].

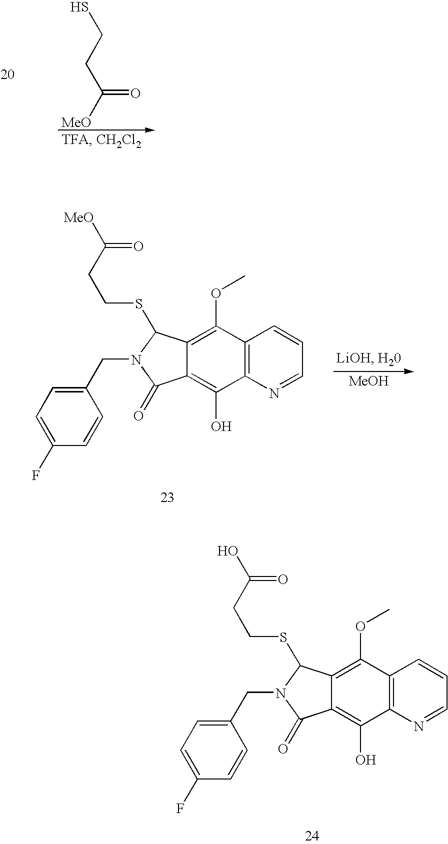

### Example 23

### 3-[7-(4-Fluoro-benzyl)-9-hydroxy-5-methoxy-8-oxo-7,8-dihydro-6H-pyrrolo[3,4-g]quinolin-6-ylsulfanyl]-propionic acid methyl ester 23

To a solution of 20 (215 mg, 0.422 mmol) in CH₂Cl₂ (4.2 mL) and TFA (98 μL, 1.26 mmol) was added methyl-3-mercaptopropionate (56 μL, 0.506 mmol). The solution was stirred at ambient temperature for 5 hours when LCMS analysis indicated complete conversion of the starting materials to the products. The solution was dried under reduced pressure and azeotroped with CH₂Cl₂ three times to provide the product 23 as a yellow solid. ¹H NMR (300 MHz, CDCl₃) δ 2.30-2.38 (m, 4H), 3.63 (s, 3H), 4.04 (s, 3H), 4.42 (d, 1H, J=15 Hz), 5.33 (d, 1H, J=15 Hz), 5.49 (s, 1H), 7.05 (t, 2H, J=9 Hz), 7.38 (dd, 2H, J=5, 8 Hz), 7.59 (dd, 1H, J=4, 9 Hz), 8.53 (d, 1H, J=8 Hz), 8.91-9.01 (m, 1H); ¹⁹F NMR (282.6 MHz, CDCl₃) δ 62.6; EI MS (m/z) 457.3 [MH⁺], 479.2 [M+Na].

### Example 24

### 3-[7-(4-Fluoro-benzyl)-9-hydroxy-5-methoxy-8-oxo-7,8-dihydro-6H-pyrrolo[3,4-g]quinolin-6-ylsulfanyl]-propionic acid 24

A solution of 23 (150 mg, 0.329 mmol) in 3.29 mL of a 1:2:3 solution of H₂O:MeOH:THF was stirred with LiOH. H₂O (69 mg, 1.65 mmol) for 1 hour when LCMS demonstrated complete conversion of starting materials to product. The reaction mixture was dried under reduced pressure and the residue was suspended in water and the pH was adjusted to 11 with aqueous 1N NaOH solution. The aqueous layer was washed with EtOAc twice. The pH of the aqueous layer was then adjusted to 5 using 1N HCl and the product was extracted with CH₂Cl₂ under continuous extraction conditions. The organic layer was dried in vacuo to yield the product 24 as an orange solid. ¹H NMR (300 MHz, CDCl₃) δ 2.1 (s, 1H), 2.25-2.45 (m, 4H), 4.04 (s, 3H), 4.43 (d, 1H, J=15 Hz), 5.32 (dd, 1H, J=3, 14 Hz), 5.49 (s, 1H), 7.03 (t, 2H, J=9 Hz), 7.35 (dd, 2H, J=5, 8 Hz), 7.57 (dd, 1H, J=4, 8 Hz), 8.52 (dd, 1H, J=2, 8 Hz), 8.98 (dd, 1H, J=2, 5H); ¹³C NMR (75 MHz, CD₃OD) δ 21.4, 33.6, 41.9, 61.8, 61.9, 112.3, 115.7, 116.0, 123.1, 125.0, 126.5, 130.4, 130.5, 131.8, 131.8, 139.3, 142.6, 148.3, 149.6, 162.4 (d, J=245 Hz), 167.2, 175.3; ¹⁹F NMR (282.6 MHz, CDCl₃) δ 62.6; EI MS (m/z) 441.4 [M−H]⁻, 883.1 [2M−2H]⁻.

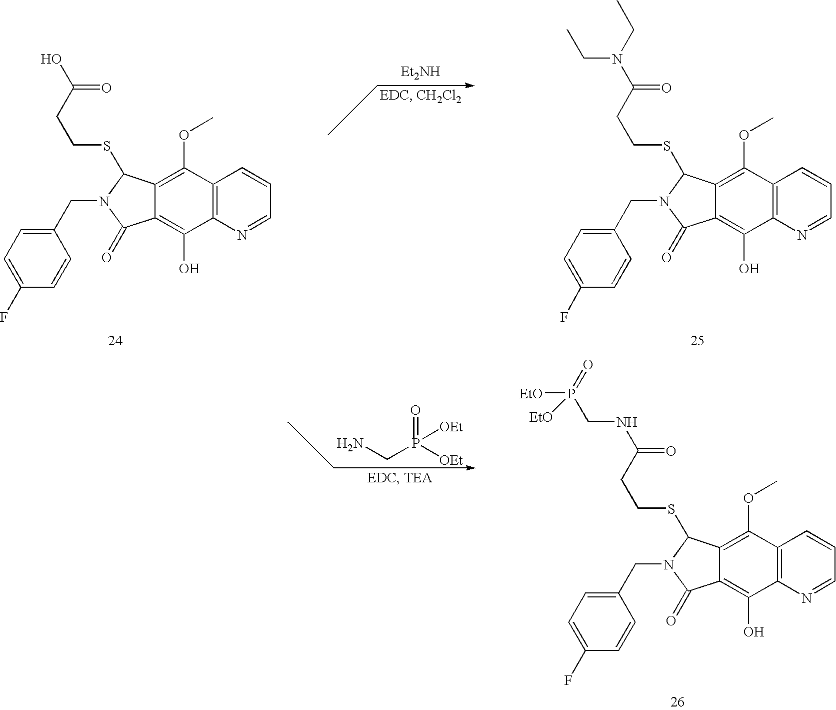

### Example 25

### N,N-Diethyl-3-[7-(4-fluoro-benzyl)-9-hydroxy-5-methoxy-8-oxo-7,8-dihydro-6H-pyrrolo[3,4-g]quinolin-6-ylsulfanyl]-propionamide 25

A solution of 24 (10.7 mg, 0.024 mmol) in CH₂Cl₂ (0.24 mL) was stirred with EDC (14 mg, 0.73 mmol) and diethyl amine (10 μL, 0.097 mmol) for 1 day at ambient temperature. The product 25 was purified by reverse phase HPLC using 5-95% A. Buffer A contained CH₃CN-1% HOAc and B contained H₂O-1% HOAc. ¹H NMR (300 MHz, CDCl₃) δ 0.984 (t, 3H, J=6 Hz), 1.05 (t, 3H, J=7 Hz), 2.23-2.45 (m, 4H), 3.04 (q, 2H, J=7 Hz), 3.29 (q, 2H, J=8 Hz), 4.06 (s, 3H), 4.47 (d, 1H, J=14 Hz), 5.31 (d, 1H, J=15 Hz), 5.50 (s, 1H), 7.05 (t, 2H, J=9 Hz), 7.36-7.44 (m, 2H), 7.55-7.62 (m, 1H), 8.53 (d, 1H, J=9 Hz), 8.95-9.00 (m, 1H); EI MS (m/z) 520.2 [MH⁺], 1016.9 [2M+Na].

### Example 26

### ({3-[7-(4-Fluoro-benzyl)-9-hydroxy-5-methoxy-8-oxo-7,8-dihydro-6H-pyrrolo[3,4-g]quinolin-6-ylsulfanyl]-propionylamino}-methyl)-phosphonic acid diethyl ester 26

To a solution of 24 (15 mg, 0.035 mmol) in 0.35 mL of CH₂Cl₂ (0.35 mL) was added diethyl(aminomethyl)phosphonate oxalate (27 mg, 0.105 mmol), EDC (20 mg, 0.105 mmol) and TEA (15 μL, 0.105 mmol). The solution was stirred at room temperature for 1 day when the same amount of the aminomethyl phosphonate, EDC and TEA were added. The reaction was stirred for another day when complete conversion of starting materials to the desired product was observed by LCMS. The product 26 was purified by reverse phase HPLC using 5-95% A. Buffer A contained CH₃CN-1% HOAc and buffer B was H₂O-1% HOAc. ¹H NMR (300 MHz, CDCl₃) δ 1.33-1.40 (m, 6H), 2.37-2.45 (m, 4H), 3.60-3.72 (m, 2H), 4.05 (s, 3H), 4.06-4.18 (m, 4H), 4.44 (d, 1H, J=15 Hz), 5.33 (d, 1H, J=14 Hz), 5.49 (s, 1H), 6.17 (br s, 1H), 6.98-7.08 (m, 2H), 7.33-7.43 (m, 2H), 7.55-7.63 (m, 1H), 8.50-8.57 (br d, 1H), 8.97 (br s, 1H); ³¹P (121.4 MHz, CDCl₃) δ 22.7; ¹⁹F NMR (282.6 MHz, CDCl₃) δ 62.6; EI MS (m/z) 590.4 [M−H]⁻, 614.2 [M+Na].

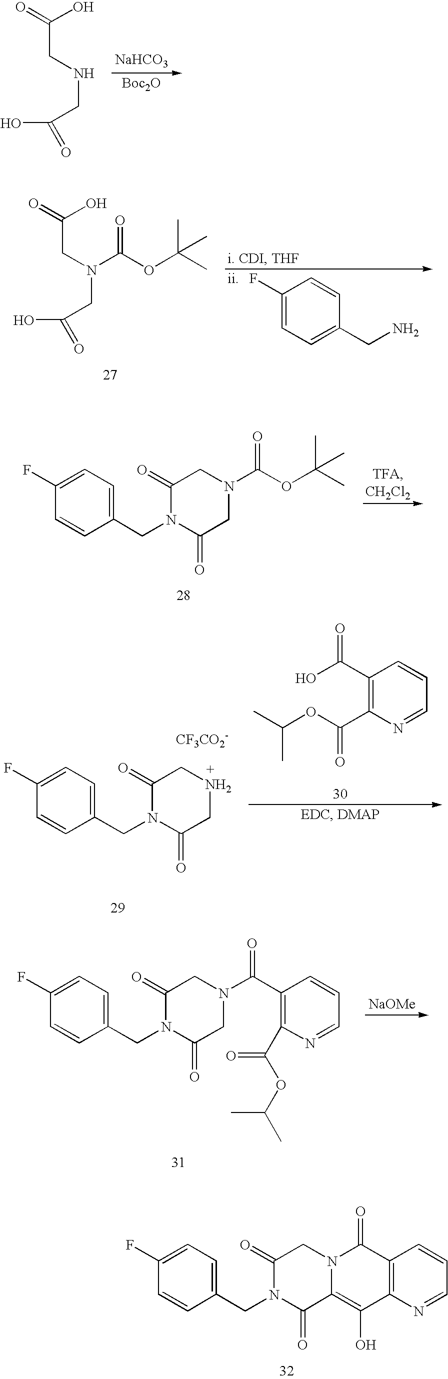

### Example 27

### (tert-Butoxycarbonyl-carboxymethyl-amino)-acetic acid 27

A mixture of iminodiacetic acid (5.1 g, 38.3 mmol) and sodium hydrogen carbonate (NaHCO₃, 12.9 g, 153 mmol) were dissolved in 50 mL of water. Once the bubbling subsided, 50 mL of THF was added followed by 10.0 g (46.0 mmol) of BOC₂O. The mixture was stirred at ambient temperature for 2 days when starting materials were completely consumed as detected by ESI. The reaction was worked up by removing THF and washing the aqueous layer with Et₂O twice. The pH of the aqueous layer was then adjusted to 1 using conc. HCl. The product was extracted with EtOAc and solvent removed in vacuo to provide the product as a white solid. The product was purified by crystallization from EtOAc to give 8.04 g (90%) of clear crystals of 27. ES MS [M−H]⁻ 232.1.

### Example 28

### 4-(4-Fluoro-benzyl)-3,5-dioxo-piperazine-1-carboxylic acid tert-butyl ester 28

A solution of 27 (547 mg, 2.35 mmol) and carbonyl diimidazole (837 mg, 5.16 mmol) in 4.7 mL of dry THF under a N₂ atmosphere was refluxed for 5 minutes. Once the reaction cooled down to room temperature 4-fluorobenzyl amine (0.295 mL, 2.58 mmol) was added and the mixture was heated to reflux overnight. The reaction mixture was then concentrated and re-dissolved in EtOAc. The organic layer was washed with an aqueous 0.5 N HCl solution and the solvent was removed in vacuo. The product was purified by column chromatography eluting with CH₂Cl₂ to provide clean product 28 as a clear oil. ¹H NMR (300 MHz, CDCl₃) δ 1.47 (s, 9H), 4.39 (s, 4H), 4.92 (s, 2H), 6.99 (t, 2H, J=9 Hz), 7.40 (dd, 2H, J=5, 9 Hz); ¹³C NMR (75 MHz, CDCl₃) δ 28.1, 42.0, 47.1, 82.3, 115.2, 115.5, 131.1, 131.2, 132.0, 153.0, 162.7 (d, J=245 Hz), 168.0; ¹⁹F NMR (282.6 MHz, CDCl₃) δ 62.5; EI MS (m/z) 340.5 [M+Na].

### Example 29

### 4-(4-Fluoro-benzyl)-3,5-dioxo-piperazin-1-ium; trifluoro-acetate 29

A solution of 28 (26 mg, 0.080 mmol) in 2 mL of CH₂Cl₂ was stirred with 1 mL of TFA for 1.5 hours when TLC indicated complete conversion to the product. The solution was dried in vacuo to yield a white solid. The product was purified by crystallization using CH₂Cl_{2.} ¹H NMR (300 MHz, CD₃OD) δ 4.18 (s, 4H), 4.95 (s, 2H), 5.01 (s, 2H), 7.01 (dt, 2H, J=2, 9 Hz), 7.41 (ddd, 2H, J=2, 5, 9 Hz); ¹⁹F NMR (282.6 MHz, CDCl₃) δ −77.5, 60.0.

### Example 30

### Pyridine-2,3-dicarboxylic acid 2-isopropyl ester 30

A mixture of 2,3-pyridine carboxylic anhydride (100 g, 0.67 mol) in 500 mL of i-PrOH was heated at reflux for 1 day according to the procedure of Ornstein, P. et al. J. Med. Chem. (1989) 32, 4, 827. The reaction mixture was then dried in vacuo to provide the product 30 as a white solid. ¹H NMR (300 MHz, CD₃OD) δ 1.37 (d, 6H, J=7 Hz), 5.27 (septet, 1H, J=6 Hz), 7.63 (dd, 1H, J=5, 8 Hz), 8.34 (dd, 1H, J=1, 8 Hz), 8.71 (d, 1H, J=5 Hz); EI MS (m/z) 210.0 [MH⁺].

### Example 31

### 3-[4-(4-Fluoro-benzyl)-3,5-dioxo-piperazine-1-carbonyl]-pyridine-2-carboxylic acid isopropyl ester 31

A solution of 29 (54 mg, 0.16 mmol), 30 (34 mg, 0.16 mmol), EDC (92 mg, 0.48 mmol), dimethylaminopyridine (20 mg, 0.16 mmol), triethylamine (67 μL, 0.48 mmol) in 1.6 mL of a 1:1 mixture of CH₂Cl₂:DMF was stirred for 1 day at ambient temperature. The reaction mixture was directly loaded onto a silica column and the product was eluted with a gradient of 1:1 Hex-EtOAc to EtOAc followed by 10% MeOH-EtOAc. The product 31 was obtained as a clear oil. EI MS (m/z) 414.7 [MH⁺], 436.4 [M+Na].

### Example 32

### 7-(4-Fluoro-benzyl)-9-hydroxy-1,7,10a-triaza-anthracene-6,8,10-trione 32

A solution of 31 (5 mg, 0.01 mmol) in 0.3 mL of dry 0.5 M NaOMe was stirred at ambient temperature for 15 minutes when a yellow precipitate formed. The solvent was removed in vacuo and the solid was dissolved in a mixture of CH₂Cl₂-1N HCl. The layers were separated and the aqueous layer was washed with CH₂Cl₂. The organic solvent was removed to provide an off-white solid. The product 32 was purified by trituration using CH₂Cl₂ and hexane. ¹H NMR (300 MHz, CDCl₃) δ 5.01 (s, 2H), 5.16 (s, 2H), 7.02 (dt, 2H, J=2, 9 Hz), 7.51 (ddd, 2H, J=2, 5, 9 Hz), 7.79 (dd, 1H, J=8, 5 Hz), 8.61 (dd, 1H, J=8, 2 Hz), 9.13 (dd, 1H, J=4, 2 Hz), 12.35 (s, 1H); ¹³C NMR (75 MHz, CDCl₃) δ 42.4, 46.1, 107.0, 115.5, 115.8, 126.7, 127.1, 130.8, 131.4, 131.5, 132.5, 143.2, 148.4, 153.7, 156.0, 162.2 (d, J=249 Hz), 163.9, 164.0; EI MS (m/z) 354.6 [MH⁺].

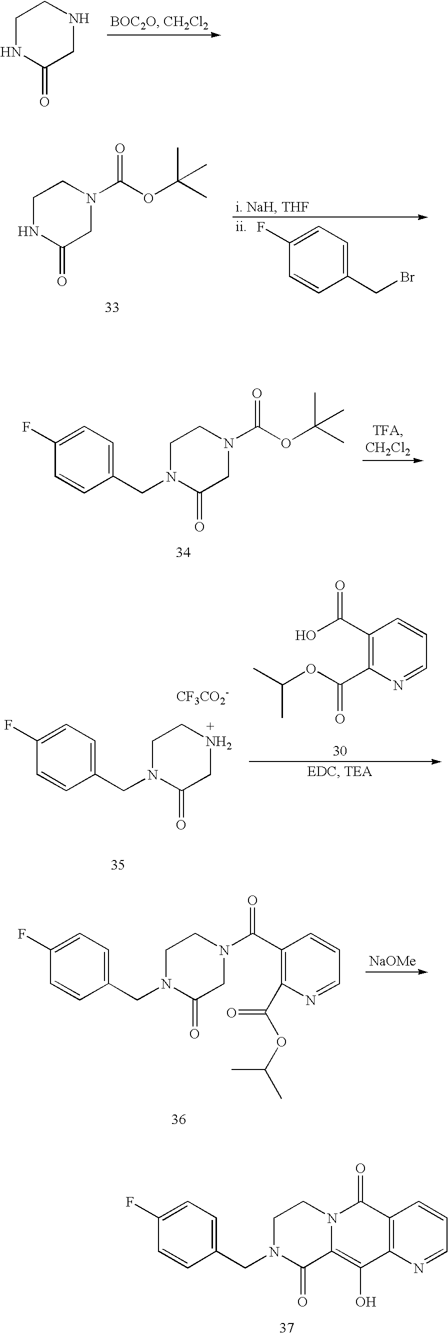

### Example 33

### 3-Oxo-piperazine-1-carboxylic acid tert-butyl ester 33

To a mixture of piperazine-2-one (1.037 g, 10.4 mmol) in 52 mL of CH₂Cl₂, was added BOC₂₀ (2.5 g, 11.4 mmol). The reaction became homogeneous after 3 hours when the starting material was completely consumed. The reaction was diluted with CH₂Cl₂ and the organic layer was washed with water. The solvent was removed in vacuo to yield quantitative amount of product 33 as a white solid. ¹H NMR (300 MHz, CDCl₃) δ 1.48 (s, 9H), 3.35-3.44 (m, 2H), 3.64 (t, 2H, J=5 Hz), 4.10 (s, 2H), 6.41 (brs, 1H).

### Example 34

### 4-(4-Fluoro-benzyl)-3-oxo-piperazine-1-carboxylic acid tert-butyl ester 34

To a heterogeneous solution of 33 (1.6 g, 8.1 mmol) in 16.2 mL of dry THF under a N₂ atmosphere was added 0.211 g (8.80 mmol) of 95% NaH. Once the bubbling subsided, 4-fluorobenzylbromide (1.2 mL, 9.7 mmol) was added dropwise to the solution. After 1 hour, when the reaction was complete as judged by TLC, the reaction was quenched by addition of water and the organic layer was diluted with EtOAc. The organic layer was washed with water and the solvent removed in vacuo. The product was purified by column chromatography using 1:1 EtOAc-Hex solvent system to provide 2.3 g (93%) of the product 34 as a white solid. ¹H NMR (300 MHz, CDCl₃) δ 1.47 (s, 9H), 3.24 (t, 2H, J=5 Hz), 3.60 (t, 2H, J=5 Hz), 4.16 (s, 2H), 4.59 (s, 2H), 7.03 (t, 2H, J=9 Hz), 7.26 (dd, 2H, J=5.8 Hz); ¹⁹F NMR (282.6 MHz, CDCl₃) δ 62.2.

### Example 35

### 4-(4-Fluoro-benzyl)-3-oxo-piperazin-1-ium trifluoroacetate salt 35

A solution of 34 (1.4 g, 4.5 mmol) in 6 mL of a 1:1 solution of CH₂Cl₂:TFA was stirred at ambient temperature for 2 hours when all of the starting materials were consumed as judged by TLC. The reaction mixtures were dried in vacuo to yield 1.5 g of 35 as a thick oil which was used in the next reaction without purification.

### Example 36

### 3-[4-(4-Fluoro-benzyl)-3-oxo-piperazine-1-carbonyl]-pyridine-2-carboxylic acid isopropyl ester 36

A solution of 35 (1.46 g, 4.55 mmol) was dissolved in 20 mL of a 1:1 solution of CH₂Cl₂:DMF. To this solution was added 0.95 g (4.55 mmol) of 30, EDC (1.74 g, 9.10 mmol) and triethylamine (1.90 mL, 13.7 mmol). The solution was stirred at room temperature for 4 hours when the reaction was complete. The solution was diluted with CH₂Cl₂ and washed with water. The organic layer was subsequently washed with aq. saturated solution of NH₄Cl and the solvent was removed. The yellow residue was purified by column chromatography using EtOAc-10% MeOH gradient to yield 1.8 g (100%) of the product 36 as a clear oil. EI MS (m/z) 400.5 [MH⁺], 422.3 [M+Na].

### Example 37

### 7-(4-Fluoro-benzyl)-9-hydroxy-6,7-dihydro-SH-1,7,10a-triaza-anthracene-8,10-dione 37

To a solution of 36 (0.900 g, 2.26 mmol) in 12 mL of dry MeOH under a N₂ atmosphere was added 12.5 mL of a 0.5 M sodium methoxide (NaOMe). The solution was stirred at ambient temperature for 2.5 hours. The reaction was worked up by removing the solvent and dissolving the residue in CH₂Cl₂. The organic layer was washed with a saturated aqueous solution of NH₄Cl and dried to provide 610 mg of the product 37 as a yellow solid. ¹H NMR (300 MHz, CDCl₃) δ 3.58 (t, 2H, J=6 Hz), 4.308 (t, 2H, J=5 Hz), 4.77 (s, 2H), 7.09 (t, 2H, J=8 Hz), 7.34 (t, 2H, J=8 Hz), 7.61 (dd, 1H, J=5, 8 Hz), 8.73 (d, 1H, J=8 Hz), 9.12 (d, 1H, J=3 Hz), 13.00 (s, 1H); ¹³C NMR (75 MHz, CDCl₃) δ 38.8, 43.9, 49.5, 111.9, 115.9, 116.2, 124.7, 130.0, 130.1, 131.0, 136.4, 146.8, 147.2, 154.7, 157.3, 163.0 (d, J=245 Hz), 163.7; ¹⁹F NMR (282.6 MHz, CDCl₃) δ 63.2; EI MS (m/z) 340.5 [MH⁺], 362.3 [M+Na].

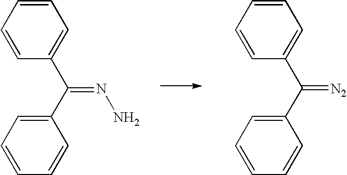

### Example 38

### Diphenyldiazomethane 38

Benzophenone hydrazone (25 g, 122.3 mmol) and sodium sulfate (anhydrous) (26 g, 183.5 mmol) were suspended in ether (anhydrous, 400 mL). To this mixture, a potassium hydroxy (powder) saturated ethanol solution (10 mL) was added, followed by mercury oxide (66.2 g, 305.8 mmol) to form a red solution. This solution was shaken at RT for 1.5 hours. The solid was filtered off. The filtrate was concentrated to a residue, which was redissolved in 200 mL of hexane and placed in a cold room overnight. The solidified solution was evaporated to dryness, which gave diphenyldiazomethane 38 as a red solid (24.7 g, 99.7%).

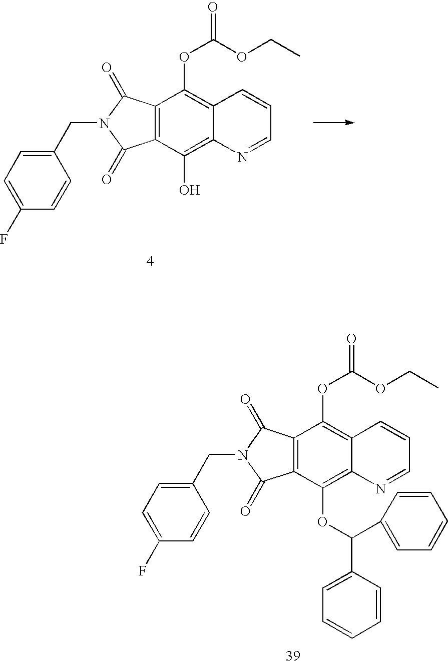

### Example 39

Mono carbonate 4 (8.9 g, 21.7 mmol) was dissolved in 1,2-dichloroethane (400 mL). Diphenyldiazomethane 38 (8.4 g, 43.4 mmol) was added in one portion. The mixture was stirred at 70° C. for 3 hours. The reaction was monitored by TLC (EtOAc/Hexane=3/7). After completion of the reaction, the solution was cooled down to room temperature. The solvent was evaporated. The crude product is chromatographed on a silica gel column, eluting with EtOAc/hexane to give the product 39 as a white solid (10.1 g, 80%). ¹H NMR (CDCl₃): δ 9.1 (d, 1H), 8.4 (d, 1H), 8.0 (s, 1H), 7.6 (dd, 1H), 7.6 (d, 4H), 7.4 (dd, 2H), 7.2-7.3 (m, 6H), 7.0 (t, 2H), 4.8 (s, 2H), 4.4 (q, 2H), 1.4 (t, 3H). MS: 577 (M+1), 599 (M+23).

The reaction was repeated, where mono-carbonate 4 (0.2 g, 0.4878 mmol) was dissolved in 9 mL of dichloroethane. To this was added diphenyldiazomethane (0.189 g, 0.9756 mmol) and stirred at 70° C. for two hours. After starting material consumed, concentrated off some solvent, and chromatographed (25% ethylacetate/hexanes) to give product 39 (0.2653 g, 0.4598 mmol, 94%.) ¹H NMR (CDCl₃) δ 9.14 (d, 1H), 8.47 (d, 1H), 7.99 (s, 1H), 7.61 (m, 5H), 7.43 (dd, 2H), 7.27 (m, 6H), 7.02 (dd, 2H), 4.82 (s, 2H), 4.45 (q, 2H), 1.47 (t, 3H.) MS: 577 (M+1)

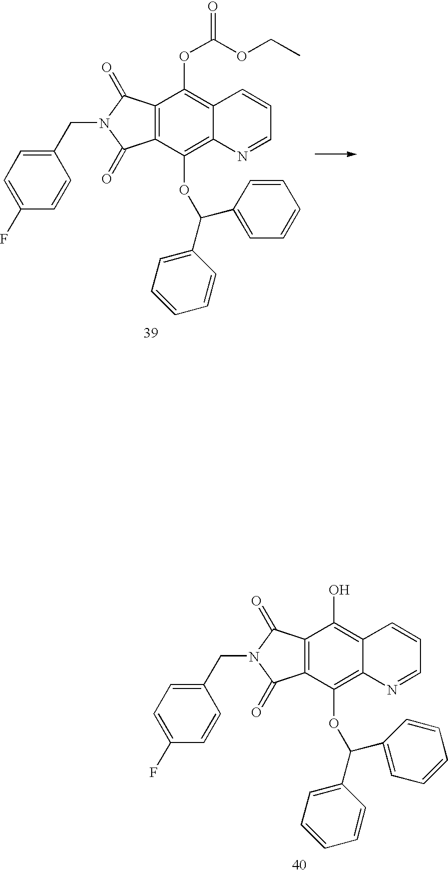

### Example 40

A solution of K₂CO₃ (24.2 g, 175.2 mmol) in water (120 mL) and 4-dimethylaminopyridine (4.24 g, 35.0 mmol) was added to the ethyl carbonate 39 (10.1 g, 17.5 mmol) in THF (180 mL). The mixture is stirred at room temperature under nitrogen atmosphere overnight. Most of THF is removed under reduced pressure at 30-40° C. and the remaining solution is diluted with dichloromethane. To this, it is acidified with 1N HCl to pH about 4. The organic phase was separated and washed with brine, dried (MgSO₄) and concentrated to give a yellow solid crude product 40 (9.9 g, 100%). ¹H NMR (CDCl₃): δ 9.1 (d, 1H), 8.6 (d, 1H), 8.4 (s, 1H, (OH)), 7.8 (s, 1H), 7.6 (dd, 1H), 7.6 (dd, 4H), 7.4 (d, 2H), 7.2-7.3 (m, 6H), 7.0 (t, 2H), 4.8 (s, 2H). LC/MS: 527 (M+23).

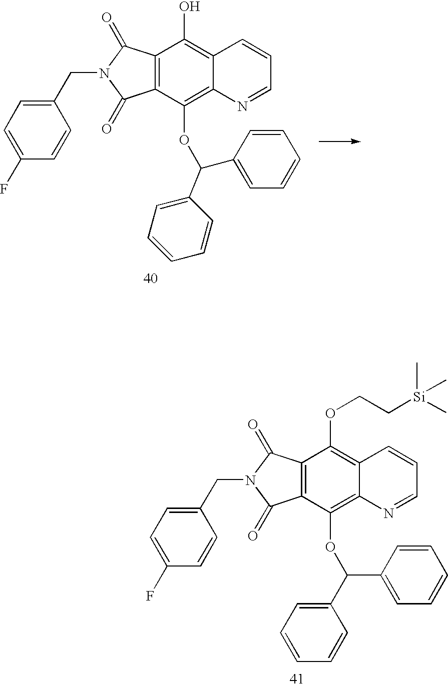

### Example 41

2-(Trimethylsilyl) ethanol (2.4 mL, 16.7 mmol), triphenylphosphine (3.5 g, 13.4 mmol) and diethyl azodicarboxylate (92.1 mL, 13.4 mmol) was added to the phenol 40 (3.37 g, 6.7 mmol) in anhydrous THF (70 mL). The solution was stirred at room temperature for 3 hours under nitrogen. TLC indicated the completion of the reaction. The solvent was evaporated and the residue oil was purified by silica gel chromatography, eluting with EtOAc/hexane to afford the product 41 (3.3 g, 82%). ¹H NMR (CDCl₃): δ 9.1 (d, 1H), 8.6 (d, 1H), 7.9 (s, 1H), 7.6 (dd, 1H), 7.6 (d, 4H), 7.4 (d, 2H), 7.2-7.3 (m, 6H), 7.0 (t, 2H), 4.8 (s, 2H), 4.6 (t, 2H), 1.2 (t, 2H). MS: 605 (M+1), 627 (M+23).

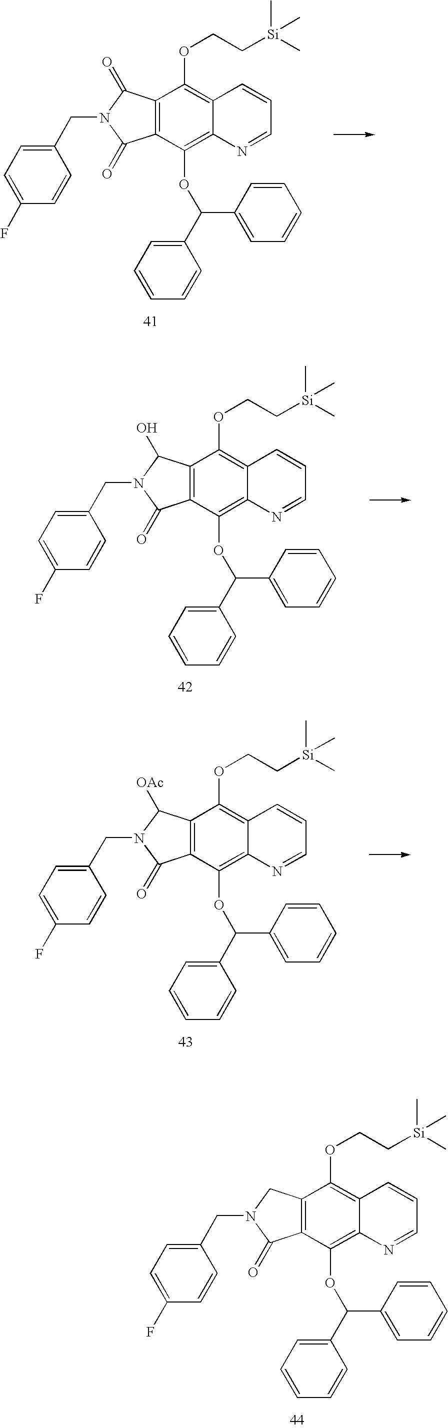

### Example 42

Compound 41 (3.3 g, 5.46 mmol) was dissolved in the mixture of THF (40 mL), isopropanol (20 mL) and water (10 mL) and chilled to 0° C. in an ice-bath. To this was added lithium borohydride (373.0 mg, 16.4 mmol) slowly. The mixture was stirred at 0° C. for 1 hour and at room temperature for 1 hour under nitrogen. TLC indicated the completion of the reaction. A solution of 1N HCl (30 mL) was added and the mixture was extracted twice with CH₂Cl₂ (2×50 mL). The organic layer was washed with saturated NaHCO₃ and dried over Mg₂SO₄ and evaporated to dryness to give 42 as an oil (3.3 g).

### Example 43

Crude product 42 was dissolved in anhydrous dichloromethane (50 mL). N-dimethylaminopyridine (66.7 mg, 0.546 mmol), N,N-diisopropylethylamine (2.85 mL, 16.4 mmol) and acetic anhydride (1.03 mL, 109 mmol) were added. The mixture was stirred at room temperature under nitrogen overnight. TLC indicated the completion of the reaction. The reaction was quenched with 1N HCl (30 mL) and extracted with CH₂Cl₂ twice (2×50 mL). The organic layer was washed with saturated NaHCO₃, dried (Mg₂SO₄) and concentrated to give crude product 43 (3.5 g).

### Example 44

Crude product 43 was dissolved in anhydrous dichloromethane (60 mL) under nitrogen. To this solution was added 2,6-lutidine (3.2 mL, 23.7 mmol), triethylsiliane (10 mL), then trimethylsilyl triflate (1.5 mL, 8.2 mmol) slowly. The mixture was stirred at room temperature for 3 hours. TLC indicated the completion of the reaction. It was quenched with 1N HCl (30 mL) and extracted with CH₂Cl₂ twice (2×50 mL). The organic layer was washed with saturated NaHCO₃, dried (Mg₂SO₄) and concentrated. The residue was chromatographed on a silica gel column, eluting with EtOAc/Hexane to afford 44 (1.4 g, 43.4% in 3 steps from 41). ¹H NMR (CDCl₃): δ 9.0 (d, 1H), 8.4 (d, 1H), 8.0 (s, 1H), 7.7 (d, 4H), 7.4 (dd, 1H), 7.1-7.3 (m, 8H), 7.0 (t, 2H), 4.8 (s, 2H), 4.2 (s, 2H), 4.1 (t, 2H), 1.1 (t, 2H), 0.1 (s, 9H). MS: 591 (M+1).

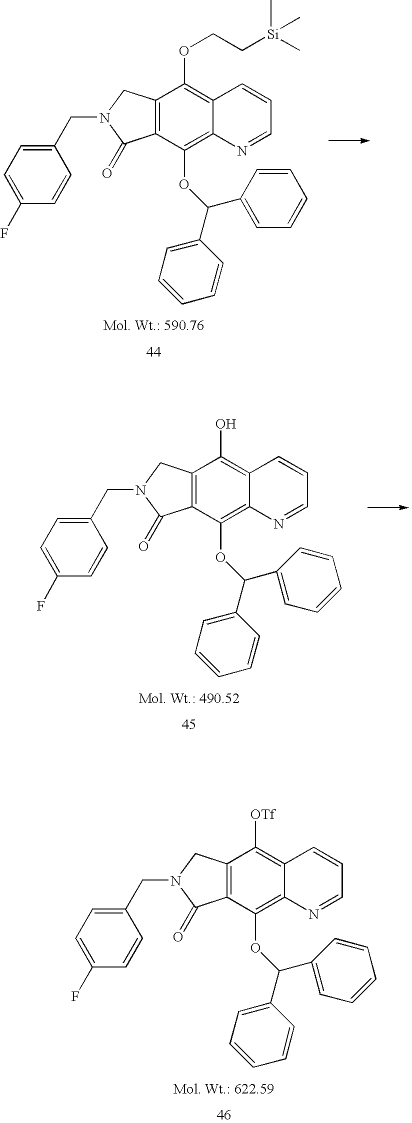

### Example 45

To 9-benzhydryloxy-7-(4-fluoro-benzyl)-5-(2-trimethylsilanyl-ethoxy)-6,7-dihydro-pyrrolo[3,4-g]quinolin-8-one 44 (300.8 mg, 0.509 mmol) in anhydrous THF (20 mL), was added tetrabutylammonium fluoride hydrate (500 mg, 1.02 mmol). The reaction mixture turned to red and was stirred at room temperature under nitrogen for 1 hour. The reaction was monitored by TLC (EtOAc/Hexane=3/7). After completion of the reaction, it was diluted with EtOAc (50 mL) and washed with 1N HCl, saturated NaHCO₃ and brine. The organic layer was dried (MgSO₄) and concentrated to give a crude product 45 (280 mg).

The reaction was repeated whereby, to a solution of lactam 44 (0.026 g, 0.044 mmol) in THF (0.441 mL) was added triethylamine (0.025 mL, 0.176 mmol) and tetrabutylammonium fluoride in 1M THF (0.066 mL). The reaction mixture was stirred at room temperature under an inert atmosphere for 30 minutes, monitored to completion by MS. The mixture was diluted with dichloromethane, washed with saturated NH₄Cl, dried (MgSO₄), and concentrated in vacuo. The crude material 45 was taken forward immediately with no further purification or characterization: MS: 491 (M+1).

Alternatively, to a solution of 9-benzhydryloxy-7-(4-fluoro-benzyl)-5-(2-trimethylsilanyl-ethoxy)-6,7-dihydro-pyrrolo[3,4-g]quinolin-8-one 44 (30 mg, 0.051 mmol) dissolved in THF (1 mL) was added tetrabutylammonium fluoride hydrate (1M in THF, 150 μl). The reaction mixture turned to red and was stirred at room temperature for ½ hours under an inert atmosphere, which generated 9-benzhydryloxy-7-(4-fluoro-benzyl)-5-hydroxy-6,7-dihydro-pyrrolo[3,4-g]quinolin-8-one 45. TLC was used to monitor the reaction.

### Example 46

Crude compound 45 was dissolved in dichloromethane (20 mL). To this was added cesium carbonate (200 mg, 0.611 mmol) and N-phenyltrifluoromethane sulfonimide (220 mg, 0.611 mmol). The mixture was stirred at room temperature under nitrogen for 16 hours. The reaction was monitored by TLC (EtOAc/Hexane=3/7). After completion of the reaction, it was diluted with EtOAc (50 mL) and washed with 1N HCl, saturated NaHCO₃ and brine. The organic layer was dried (MgSO₄) and concentrated. The residue was chromatographed on a silica gel column, eluting with EtOAc/Hexane to afford the clean product 46 (135 mg, 42.6% in 2 steps). ¹H NMR (CDCl₃): δ 9.1 (d, 1H), 8.3 (d, 1H), 8.0 (s, 1H), 7.7 (d, 4H), 7.6 (dd, 1H), 7.2-7.4 (m, 8H), 7.1 (t, 2H), 4.8 (s, 2H), 4.4 (s, 2H). MS: 623 (M+1), 645 (M+23).

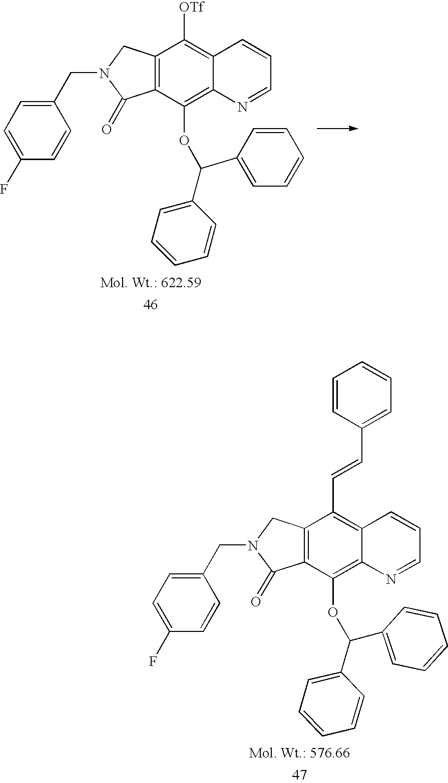

### Example 47

To the triflate 46 (66.6 mg, 0.107 mmol) in toluene (2.8 mL)/ethanol (1.2 mL)/water (0.8 mL) was added potassium carbonate (37 mg, 0.268 mmol), trans-phenylvinylboronic acid (24.5 mg, 0.160 mmol) and tetrakis(triphenylphosphine)-palladium (0) (18.5 mg, 0.016 mmol). The mixture in the flask was flushed with argon three times and heated to 120° C. under argon for 3 hours. The mixture was cooled to room temperature, diluted with EtOAc and washed with 1N HCl, saturated NaHCO₃ and brine. The organic phase was dried (MgSO₄) and concentrated. The residue was chromatographed on a silica gel column, eluting with EtOAc/Hexane to afford the product 47 (51.4 mg, 83%). ¹H NMR (CDCl₃): δ 9.0 (d, 1H), 8.4 (d, 1H), 8.1 (s, 1H), 7.7 (d, 4H), 7.2-7.5 (m, 14H), 7.1 (d, 1H), 7.0 (dd, 2H), 6.8 (d, 1H), 4.8 (s, 2H), 4.4 (s, 2H). MS: 577 (M+1), 599 (M+23).

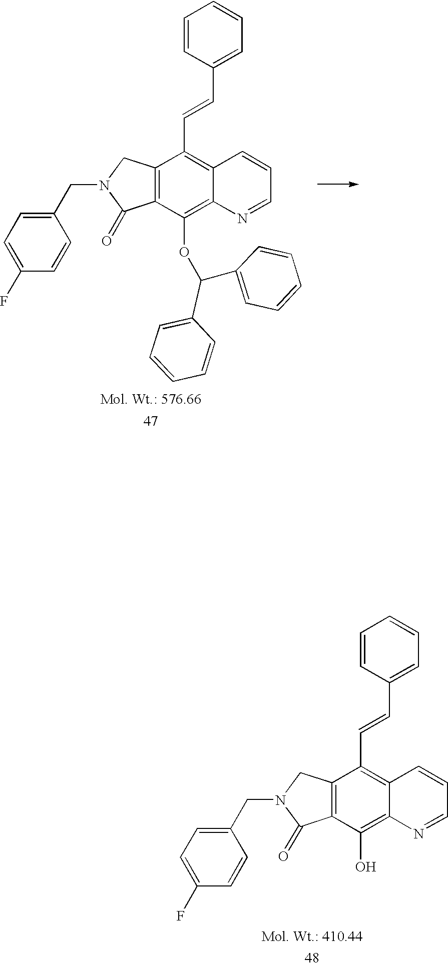

### Example 48

The compound 47 (12 mg, 0.02 mmol) was dissolved in dichloromethane (1 mL) at room temperature under nitrogen. Triethylsilane (200 μL) was added followed by TFA (100 μL) slowly. The mixture became smoke and dark. It was stirred at room temperature for 30 min. The solvent was removed under reduced pressure. The crude product was triturated in diethylether/hexane to afford a yellow solid 48 (9 mg, 90%). ¹H NMR (CDCl₃): δ 9.0 (d, 1H), 8.6 (d, 1H), 7.5 (m, 3H), 7.2-7.4 (m, 6H), 7.1 (m, 2H), 6.8 (d, 1H), 4.8 (s, 2H), 4.5 (s, 2H). MS: 411 (M+1).

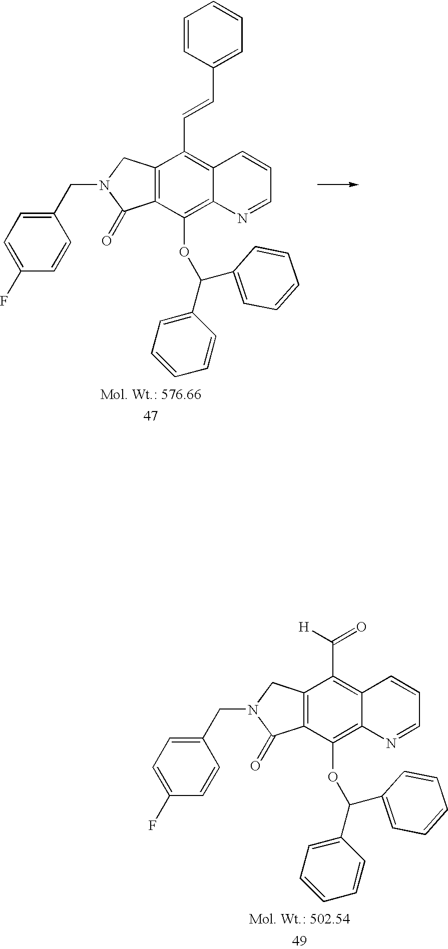

### Example 49

Compound 47 (405 mg, 0.7 mmol) in dichloromethane (150 mL) was chilled to −78° C. Ozone (03) was passed slowly into the solution over 30 min. TLC indicated the completion of the reaction. Nitrogen was bubbled into the mixture for 10 min to expel excess 03. Dimethyl sulfate (10 mL) was then added the mixture at −78° C. and the mixture was warmed to room temperature slowly with stirring. After 16 hours, the mixture was evaporated to dryness and the residue was purified by chromatography on a silica gel column, eluting with methanol/dichloromethane to give product of 49 (166.5 mg) and its hydrate form (122 mg), total yield of 80.8%. ¹H NMR (CDCl₃): δ 10.7 (s, 1H, CHO), 9.1 (m, 2H), 8.4 (s, 1H), 7.7 (d, 4H), 7.6 (dd, 2H), 7.2-7.4 (m, 8H), 7.0 (t, 2H), 4.8 (s, 2H), 4.6 (s, 2H). MS: 503 (M+1), 525 (M+23).

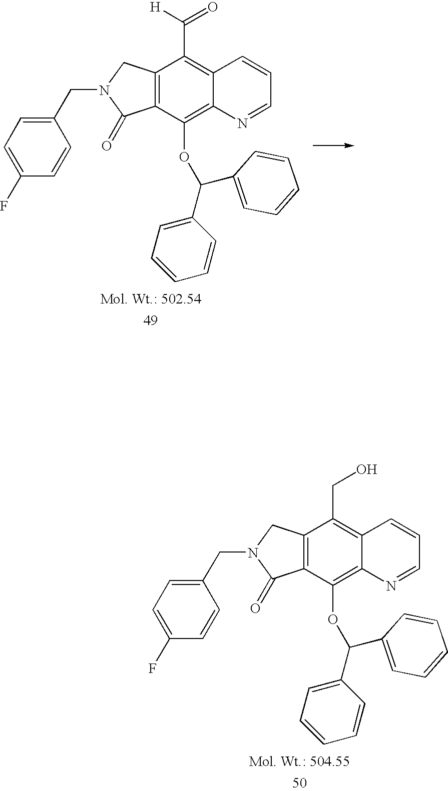

### Example 50

The aldehyde 49 (23 g, 0.046 mmol) was dissolved in anhydrous THF (1 mL) and MeOH (0.1 mL) at room temperature. To this was added sodium borohydride (5.2 mg, 0.14 mmol) slowly. The mixture was stirred at room temperature for 30 min under nitrogen. TLC indicated the completion of the reaction. The mixture was diluted with water (5 mL). The insoluble material was collected by filtration and washed with hexane and air-dried to give product 50 (13.5 mg, 59%). ¹H NMR (CD₃OD): δ 9.3 (d, 1H), 9.1 (d, 1H), 8.1 (dd, 1H), 8.0 (s, 1H), 7.5 (d, 4H), 7.4 (dd, 2H), 7.3 (m, 6H), 7.1 (t, 2H), 5.0 (s, 2H), 4.9 (s, 2H), 4.7 (s, 2H). MS: 505 (M+1), 527 (M+23).

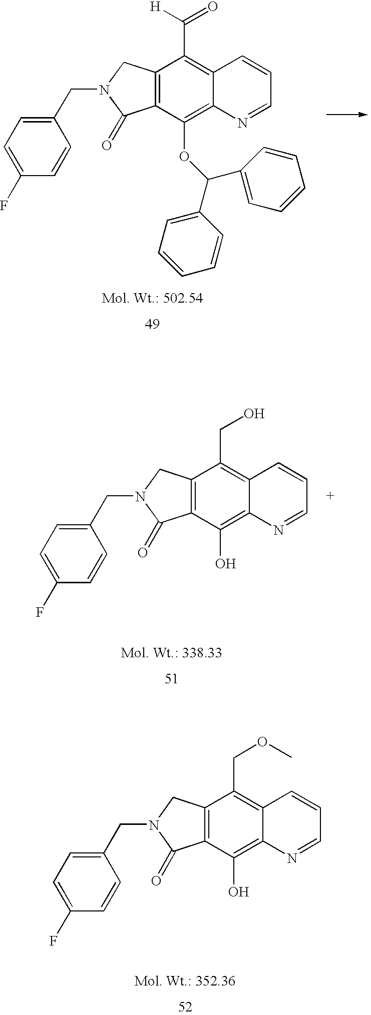

### Example 51

The aldehyde 49 (121 mg, 0.24 mmol) was dissolved in anhydrous THF (5 mL) and MeOH (0.5 mL) at room temperature. To this was added sodium borohydride (27 mg, 0.72 mmol) slowly. The mixture was stirred at room temperature for 30 min under nitrogen. It was diluted with 1N HCl (10 mL), and stirred for 10 min. The phases were separated and the aqueous phase was lyophilized to give a yellow solid, which was washed with water and ether. The solid was dried to give 50 mg of product 51. ¹H NMR (DMSO-d₆): δ 9.0 (d, 1H), 8.8 (d, 1H), 7.5 (m, 1H), 7.4 (m, 2H), 7.2 (m, 2H), 5.0 (s, 1H, PhOH), 4.8 (s, 2H), 4.7 (s, 2H), 4.5 (s, 2H). MS: 339 (M+1).

The organic phase was concentrated. The residue was dissolved in DMF (2 mL) I and purified by Prep-HPLC to give 10 mg of product 52. HPLC condition: mobile phase A (1% AcOH in water), mobile phase B (1% AcOH in AcCN); gradient: 20% to 50% B in 30 min; flow rate: 20 mL/min; column: Phenomenex, Luna 5μ, C18 (2), 150 mm×21.2 mm. ¹H NMR (DMSO-d₆): δ 9.5 (d, 1H), 89.0 (d, 1H), 7.7 (m, 1H), 7.3 (m, 2H), 7.2 (m, 2H), 4.7 (s, 2H), 4.6 (s, 2H), 4.5 (s, 2H), 3.5 (s, 3H, under water peak). MS: 353 (M+1).

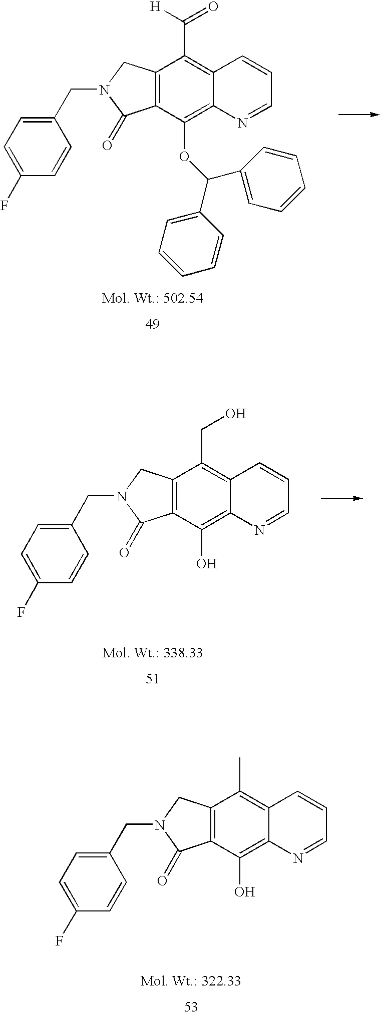

### Example 52

The aldehyde 49 (118 mg, 0.23 mmol) was dissolved in anhydrous THF (5 mL) and MeOH (0.5 mL) at room temperature. To this was added sodium borohydride (27 mg, 0.72 mmol) slowly. The mixture was stirred at room temperature for 30 min under nitrogen. It was diluted with 1N HCl (10 mL), and stirred for 10 min. The phases were separated and the aqueous phase was lyophilized to give a yellow solid as product 51.

The alcohol 51 (crude from reduction) was suspended in dichloromethane (10 mL) at room temperature under nitrogen. Triethylsilane (3 mL) was added followed by TFA (1 mL) slowly. The mixture became homogeneous and was stirred at room temperature overnight under nitrogen. The solvent was removed under reduced pressure. The crude product was dissolved in 2 mL of DMF then purified by prep-HPLC to gave a clean product of 53 (22.4 mg, 30%). HPLC condition: mobile phase A (1% TFA in water), mobile phase B (1% TFA in AcCN); gradient: 5% to 100% B in 20 min; flow rate: 20 mL/min; column: Phenomenex, Luna 5μ, C18 (2), 150 mm×21.2 mm. ¹H NMR (CD₃OD): δ 9.0 (d, 1H), 8.9 (d, 1H), 7.9 (dd, 1H), 7.4 (d, 4H), 7.1 (t, 2H), 4.8 (s, 2H), 4.9 (s, 2H), 4.5 (s, 2H), 2.5 (s, 3H). MS: 323 (M+1).

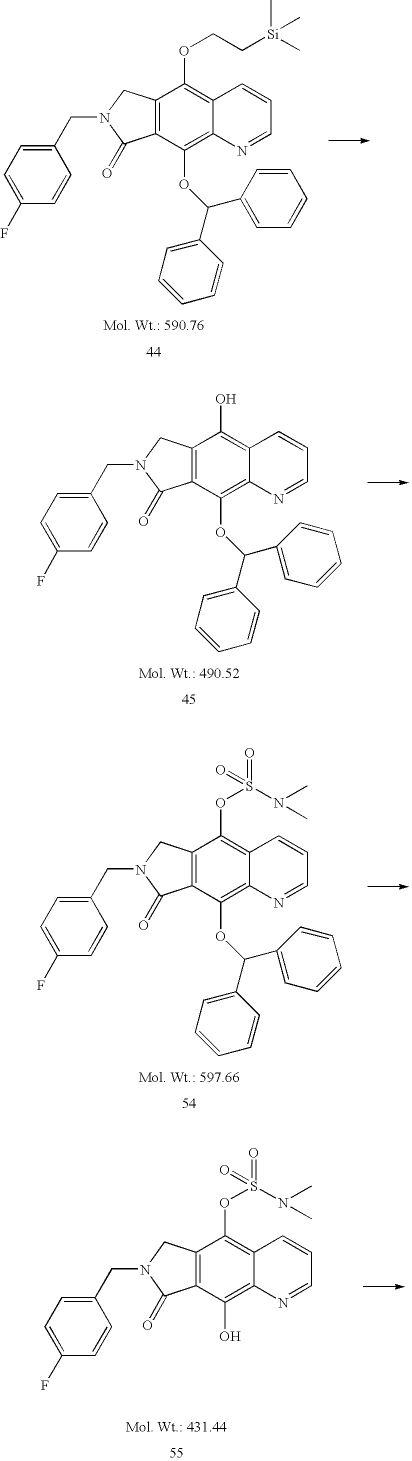

### Example 53

To the compound 44 (350.0 mg, 0.592 mmol) in anhydrous THF (20 mL), was added tetrabutylammonium fluoride (1M in THF, 651 μl, 0.651 mmol) and triethylamine (330 μl, 2.37 mmol). The reaction mixture turned to red and was stirred at room temperature under nitrogen for 1 hour. The reaction forming 45 was monitored by TLC (EtOAc/Hexane=3/7).

### Example 54

Triethylamine (330 μl, 2.37 mmol) was added to the reaction mixture followed by a catalytic amount of DMAP, and N,N-dimethylsulfamoyl chloride (160 μl, 1.5 mmol). The mixture was stirred at room temperature under nitrogen for 16 hours. After completion of the reaction, it was diluted with dichloromethane (50 mL) and washed with 1N HCl, saturated NaHCO₃ and brine The organic layer was dried (MgSO₄) and concentrated. The residue was chromatographed on a silica gel column, eluting with EtOAc/Hexane to afford the product 54 (205.4 mg, 58% in 2 steps). ¹H NMR (CDCl₃): δ 9.0 (d, 1H), 8.4 (d, 1H), 8.0 (s, 1H), 7.7 (d, 4H), 7.5 (dd, 1H), 7.1-7.3 (m, 8H), 7.0 (t, 2H), 4.8 (s, 2H), 4.4 (s, 2H), 3.0 (s, 3H). MS: 598 (M+1).

### Example 55

The compound 54 ((205.4 mg, 0.344 mmol) was dissolved in dichloromethane (6 mL) at room temperature under nitrogen. Triethylsilane (2 mL) was added followed by TFA (1 mL) slowly. The mixture became smoky and dark and was stirred at room temperature for 30 min. The solvent was removed under reduced pressure. The crude product was triturated in diethylether/hexane to afford a yellow solid 55, 169 mg, 93%.

¹H NMR (CD₃OD): δ 9.0 (d, 1H), 8.6 (d, 1H), 7.8 (dd, 1H), 7.4 (m, 2H), 7.1 (m, 2H), 4.8 (s, 2H), 4.6 (s, 2H), 3.1 (s, 6H). MS: 432 (M+1).

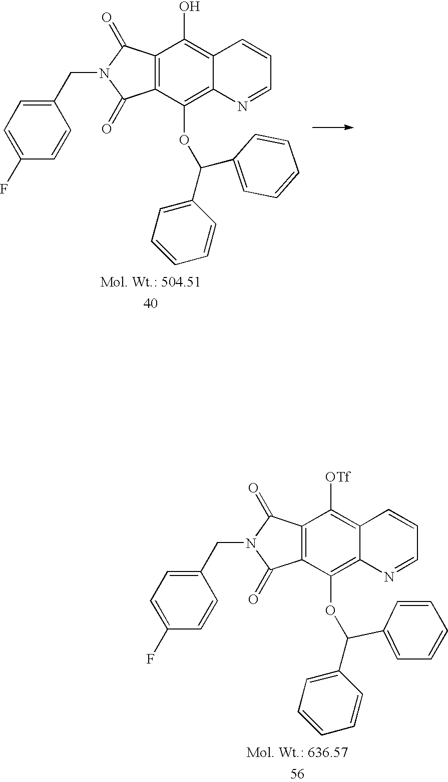

### Example 56

The phenol 40 (1.0 g, 1.984 mmol) and DIEA (1.04 mL, 6.0 mmol) in dichloromethane (20 mL) was chilled to −78° C. To this was added trifluoromethanesulfonic anhydride (0.78 mL, 3.0 mmol) slowly under the nitrogen. The reaction was completed in 1 hour. It was quenched with 1.5 mL of methanol and stirred for 5 min more. Warmed to room temperature, it was washed with 1N HCl, saturated NaHCO₃ and brine. The organic phase was dried (MgSO₄) and concentrated to afford the forming product 56 (1.2 g, 95%).

The reaction was repeated, where monophenol 40 (0.1807 g, 0.358 mmol) was dissolved in 4 mL dry dichloromethane. To this was added diisopropylethylamine (0.182 mL, 1.074 mmol.) After cooling to −78° C., triflic anhydride was added (0.14 mL, 0.537 mmol) and was stirred at this temperature for twenty minutes. Reaction was then complete by TLC, diluted with dichloromethane, washed with 1M HCl, saturated NaHCO₃ solution, dried (MgSO₄) and organics concentrated to give product (0.2518 g, 0.396 mmol, 100%) which was stored crude as a solution in 10 mL dry benzene. ¹H NMR (CDCl₃) δ 9.2 (dd, 1H), 8.46 (d, 1H), 8.068 (s, 1H), 7.75 (dd, 1H), 7.6 (d, 4H), 7.47 (dd, 1H), 7.27 (m, 7H), 7.19, dd, 2H), 4.87 (s, 2H.) MS: 637 (M+1)

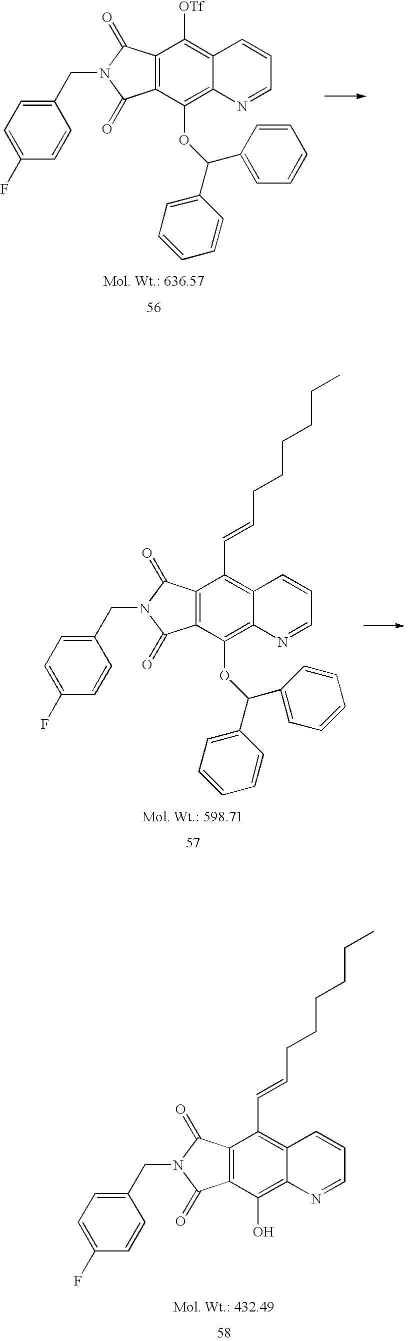

### Example 57

To the triflate 56 (78.0 mg, 0.122 mmol) in toluene (2.8 mL)/ethanol (1.2 mL)/water (0.8 mL) was added potassium carbonate (42 mg, 0.306 mmol), 1-octeneboronic acid (29.0 mg, 0.184 mmol) and tetrakis (triphenylphosphine)-palladium (0) (21.0 mg, 0.018 mmol). The mixture in the flask was flushed with argon three times. It was heated to 120° C. under argon for 3 hours. Cooling to room temperature, it was diluted with EtOAc and washed with 1N HCl, saturated NaHCO₃ and brine. The organic phase was dried (MgSO₄) and concentrated. The residue was chromatographed on a silica gel column, eluting with EtOAc/Hexane to afford the product 57 (11.4 mg, 15.6%).

### Example 58

The compound 57 (6 mg, 0.01 mmol) was dissolved in dichloromethane (1 mL) at room temperature under nitrogen. Triethylsilane (200 μL) was added followed by TFA (100 μL) slowly. The mixture became smoky and dark and was stirred at room temperature for 30 min. The solvent was removed under reduced pressure. The crude product was triturated in diethylether/hexane to afford a yellow solid TFA salt of 58, 3 mg, 57%. ¹H NMR (CD₃OD): δ 9.0 (d, 1H), 8.8 (d, 1H), 7.8 (dd, 1H), 7.4 (dd, 2H), 7.1 (d, 1H), 7.0 (dd, 2H), 6.2 (m, 1H), 4.8 (s, 2H), 2.4 (m, 2H), 1.6 (m, 2H), 1.3-1.5 (m, 6H), 0.9 (t, 3H). MS: 433 (M+1).

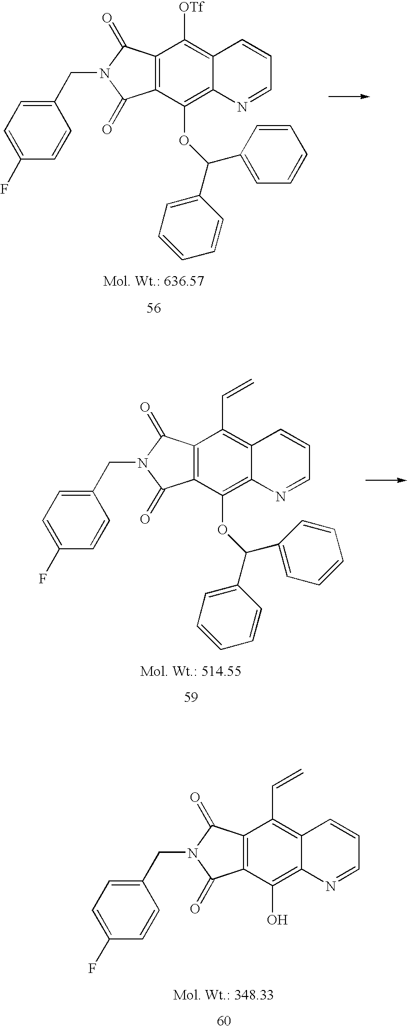

### Example 59

To the triflate 56 (100 mg, 0.157 mmol) in toluene (2.8 mL)/ethanol (1.2 mL)/water (0.8 mL) was added potassium carbonate (54 mg, 0.392 mmol), vinylboronic acid (17 mg, 0.235 mmol) and tetrakis (triphenylphosphine)-palladium (0) (27.0 mg, 0.023 mmol). The mixture in the flask was flushed with argon three times. It was heated to 120° C. under argon for 3 hours. Cooling to room temperature, it was diluted with EtOAc and washed with 1N HCl, saturated NaHCO₃ and brine. The organic phase was dried (MgSO₄) and concentrated. The residue was chromatographed on a silica gel column, eluting with EtOAc/Hexane to afford the product 59 (32.3 mg, 40%).

### Example 60

The compound 59 (11 mg, 0.01 mmol) was dissolved in dichloromethane (1 mL) at room temperature under nitrogen. Triethylsilane (200 μL) was added followed by TFA (100 μL) slowly. The mixture became smoky and dark and was stirred at room temperature for 30 min. The solvent was removed under reduced pressure. The crude product was triturated in diethylether/hexane to afford a yellow solid TFA salt of 60, 2.3 mg, 31.4%. ¹H NMR (CDCl₃): δ 9.0 (d, 1H), 8.8 (d, 1H), 7.7 (dd, 1H), 7.5 (m, 2H), 7.0 (m, 2H), 6.0 (d, 1H), 5.6 (d, 1H), 5.3 (s, 1H, OH), 4.8 (s, 2H). MS: 349 (M+1).

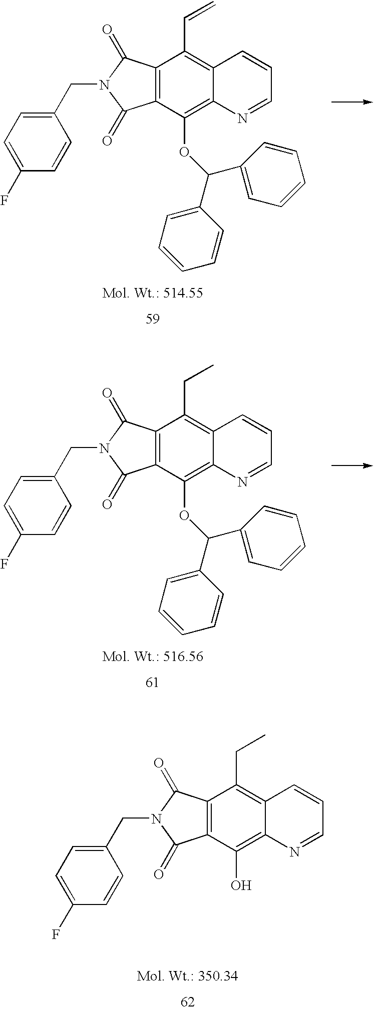

### Example 61

The compound 59 (157 g, 0.11 mmol) was dissolved in anhydrous THF (5 mL) and MeOH (0.5 mL) at room temperature. To this was added sodium borohydride (13 mg, 0.33 mmol) slowly. The mixture was stirred at room temperature for 1 hour under nitrogen. It was diluted with EtOAc (50 mL), and washed with 1N HCl, saturated NaHCO₃ and brine. The organic phase was dried (MgSO₄) and concentrated. The residue was purified by silica gel prep-TLC, eluting with EtOAc/Hexane (3/7) to afford the product 61 (12.5 mg, 22%).

### Example 62

The compound 61 (11 mg, 0.01 mmol) was dissolved in dichloromethane (1 mL) at room temperature under nitrogen. Triethylsilane (200 μL) was added followed by TFA (100 μL) slowly. The mixture became smoky and dark and was stirred at room temperature for 30 min. The solvent was removed under reduced pressure. The crude product was triturated in diethylether/hexane to afford a yellow solid TFA salt of 62, 8 mg, 75%. ¹H NMR (CDCl₃): δ 9.0 (d, 1H), 8.5 (d, 1H), 7.7 (dd, 1H), 7.5 (dd, 2H), 7.0 (m, 2H), 4.8 (s, 2H), 3.5 (q, 2H), 1.3 (t, 3H). MS: 451 (M+1).

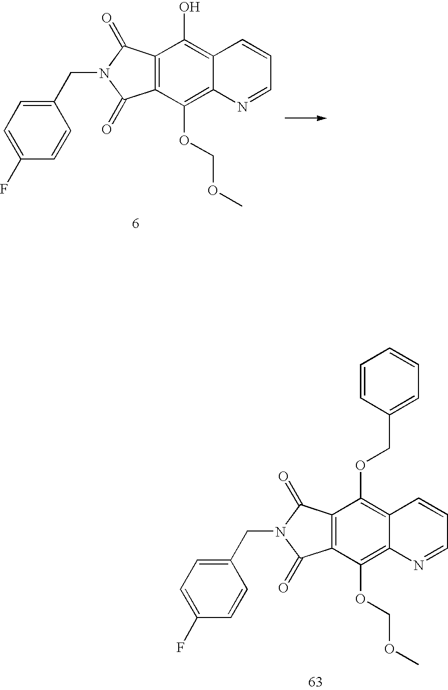

### Example 63

Mono-phenol 6 (0.02 g, 0.052 mmol) was added to 1.5 mL dry dimethylformamide. To this was added benzyl bromide (0.0124 ml, 0.104 mmol) and K₂CO₃ (0.0215 g, 0.156 mmol) and stirred at 50° C. After 1.5 hrs reaction completed by TLC. Diluted with 100 mL ethylacetate, washed with saturated NH₄Cl solution and brine. The organic phase was dried (MgSO₄), concentrated, and chromatographed (25% ethylacetate/hexanes) to give product 63 (0.013 g, 0.0275 mmol, 53%.). ¹H NMR (CDCl₃) δ 9.03 (dd, 1H), 8.6 (d, 1H), 7.54 (m, 6H), 7.4 (m, 2H), 7.05 (dd, 2H), 5.8 (s, 2H), 5.6 (s, 2H), 4.9 (s, 2H), 3.7 (s, 3H). MS: 473 (M+1)

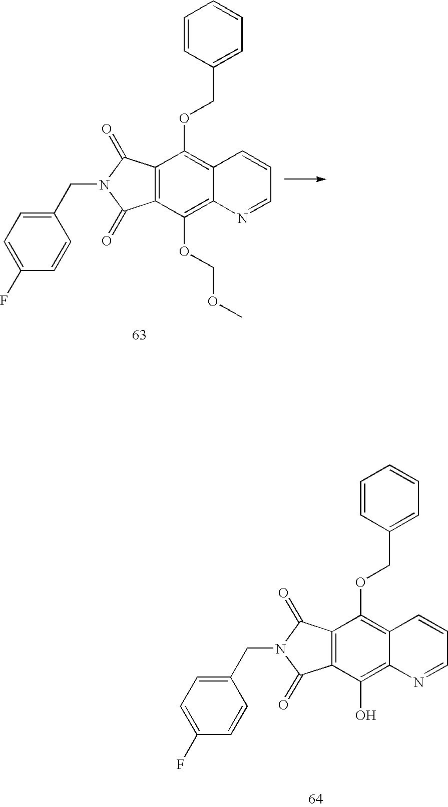

### Example 64

Benzyl ether (0.013 g, 0.0275 mmol) was dissolved in 1 mL dry dichloromethane. To this was added trifluoroacetic acid (0.0213 mL, 0.275 mmol) and stirred 2.5 hrs. Concentrated off volatiles, azeotroped with toluene (2×), concentrated to give crude product. Triturated with 1:1 diethylether/hexanes to give product 64 (0.0078 g, 0.0182 mmol, 66%). ¹H NMR (CDCl₃) δ 8.96 (dd, 1H), 8.6 (d, 1H), 7.6 (dd, 1H), 7.5 (m, 5H), 7.37 (m, 2H), 7.05 (dd, 2H), 5.6 (s, 2H), 4.88 (s, 2H). MS: 429 (M+1), 427 (M−1)

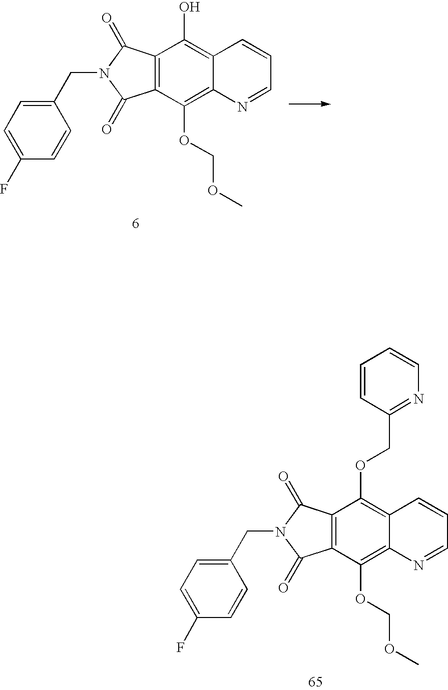

### Example 65

Monophenol 6 (0.04 g, 0.1047 mmol) was dissolved in 2 mL of dry dimethylformamide. To this was added 2-bromomethylpyridine HBr salt (0.0529 g, 0.209 mmol) and K₂CO₃ (0.144 g, 1.047 mmol.) Stirred at 50° C. for twelve hours. Diluted with ethylacetate, washed with brine (saturated NaCl) and 1M HCl, dried (MgSO₄,), and concentrated. The crude product was chromatographed (20 to 50% ethylacetate/hexanes) to give product 65: (0.0032 g, 0.0067 mmol, 6.5%.) ¹H NMR (CDCl₃) δ 9.03 (d, 1H), 8.72 (d, 1H), 8.6 (d, 1H), 7.8 (dd, 1H), 7.7 (dd, 1H), 7.57 (dd, 1H), 7.48 (dd, 2H), 7.0 (dd, 2H), 5.8 (s, 2H), 5.65 (s, 2H), 4.86 (s, 2H), 3.72 (s, 3H.) MS: 488 (M+1)

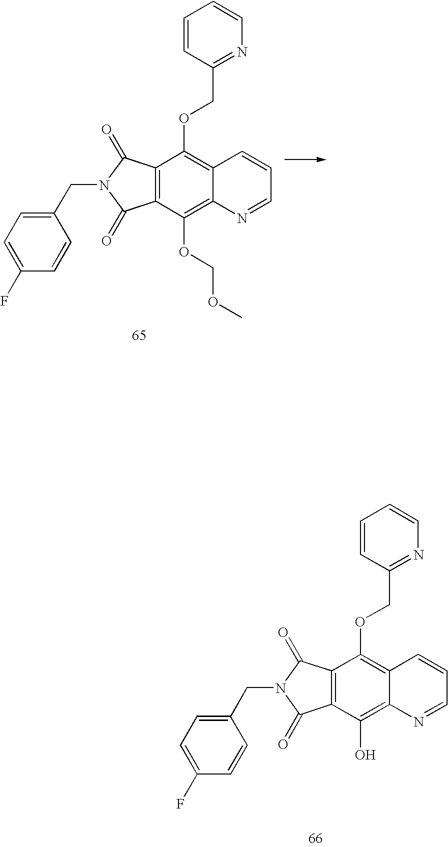

### Example 66

Pyridyl ether 65 (0.0032 g, 0.0067 mmol) was dissolved in 1 mL dry dichloromethane. To this was added trifluoroacetic acid (0.0052 mL, 0.0676 mmol) and stirred 12 hrs. Concentrated off volatiles, azeotroped with toluene (2×), concentrated to give crude product. Triturated with 1:1 diethylether/hexanes to give product 66 (0.0012 g, 0.0028 mmol, 42%.) ¹H NMR (CDCl₃) δ 8.96 (d, 1H), 8.73 (d, 1H), 8.6 (d, 1H), 7.8 (dd, 1H), 7.7 (d, 1H), 7.63 (dd, 1H), 7.5 (dd, 2H), 7.3 (m, 1H), 7.04 (dd, 2H), 5.67 (s, 2H), 4.87 (s, 2H.) MS: 430 (M+1), 428 (M−1)

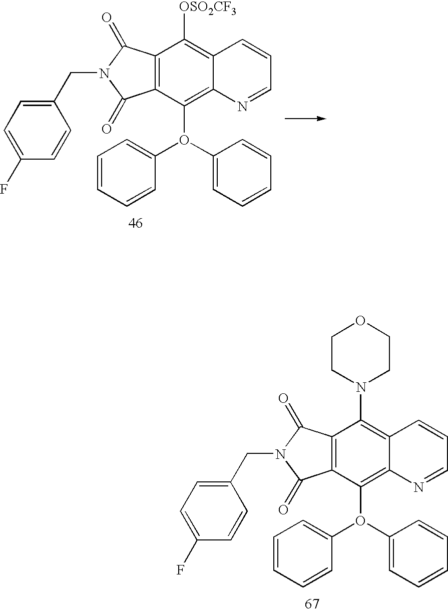

### Example 67

Triflate 46 in benzene was concentrated to give (0.0225 g, 0.0353 mmol) and dissolved in 3 mL of dichloroethane. To this was added triethylamine (0.0073 mL, 0.0529 mmol) and morpholine (0.0092 ml, 0.118 mmol) and reaction stirred at 65° C. After 15 hrs, reaction still incomplete by TLC, added another 0.118 mL of morpholine. After 21 hrs reaction time concentrated off volatiles and chromatographed (10 to 25% ethylacetate/hexanes) to give product 67 (0.0061 g, 0.01, 30%). ¹H NMR (CDCl₃) δ 9.09 (dd, 1H), 8.89 (d, 1H), 8.03 (s, 1H), 7.65 (m, 5H), 7.49 (dd, 1H), 7.27 (m, 7H), 7.06 (dd, 2H), 4.85 (s, 2H), 3.92 (dd, 4H), 3.92 (br m, 4H). MS: 574 (M+1)

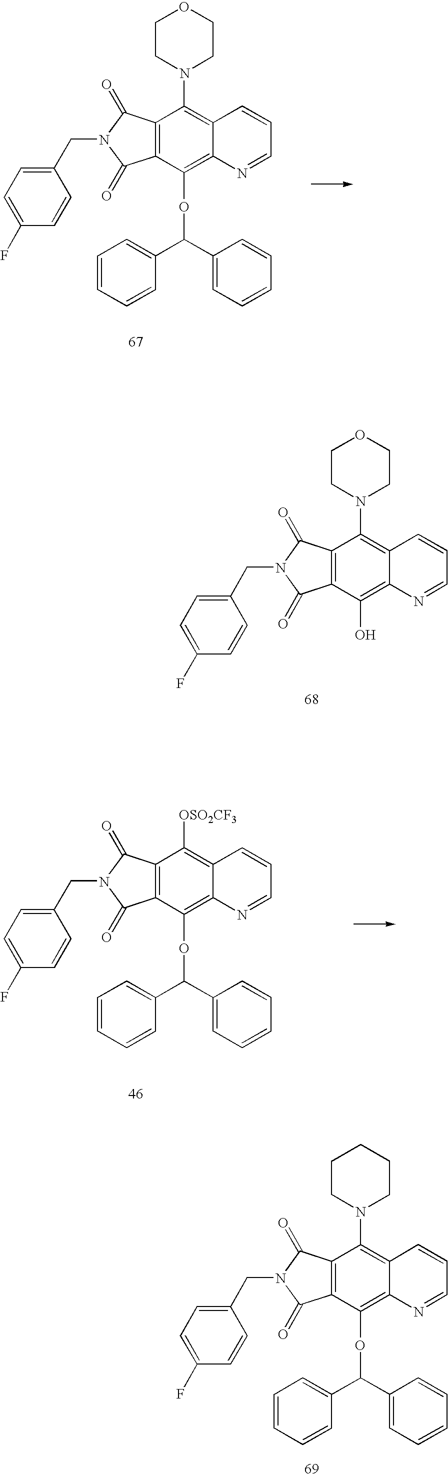

### Example 68

Tertiary amine 67 was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene, solidified with hexane and concentrated to give crude. Triturated with 1:1 diethylether/hexanes to give product 68 (0.002 g, 0.0049 mmol, 49%). ¹H NMR (CDCl₃) δ 8.98 (m, 2H), 7.7 (dd, 1H), 7.53 (dd, 2H), 7.05 (dd, 2H), 4.86 (s, 2H), 3.96 (dd, 4H), 3.35 (br m, 4H). MS: 408 (M+1), 406 (M−1)

### Example 69

Triflate 46 in benzene was concentrated to give (0.045 g, 0.0706 mmol) and dissolved in 3 mL of dichloroethane. To this was added triethylamine (0.0147 mL, 0.1059 mmol) and morpholine (0.0209 ml, 0.2118 mmol) and reaction stirred at 70° C. After 15 hrs of stirring, concentrated off volatiles and chromatographed (8 to 10% ethylacetate/hexanes) to give product 69 (0.0085 g, 0.01488, 44%.) ¹H NMR (CDCl₃) δ 9.068 (dd, 1H), 8.79 (d, 1H), 7.8 (s, 1H), 7.6 (d, 4H), 7.57 (dd, 1H), 7.46 (dd, 2H), 7.27 (m, 6H), 7.06 (dd, 2H), 4.84 (s, 2H), 3.24 (br s, 4H), 1.73 (br s, 6H.) MS: 572 (M+1)

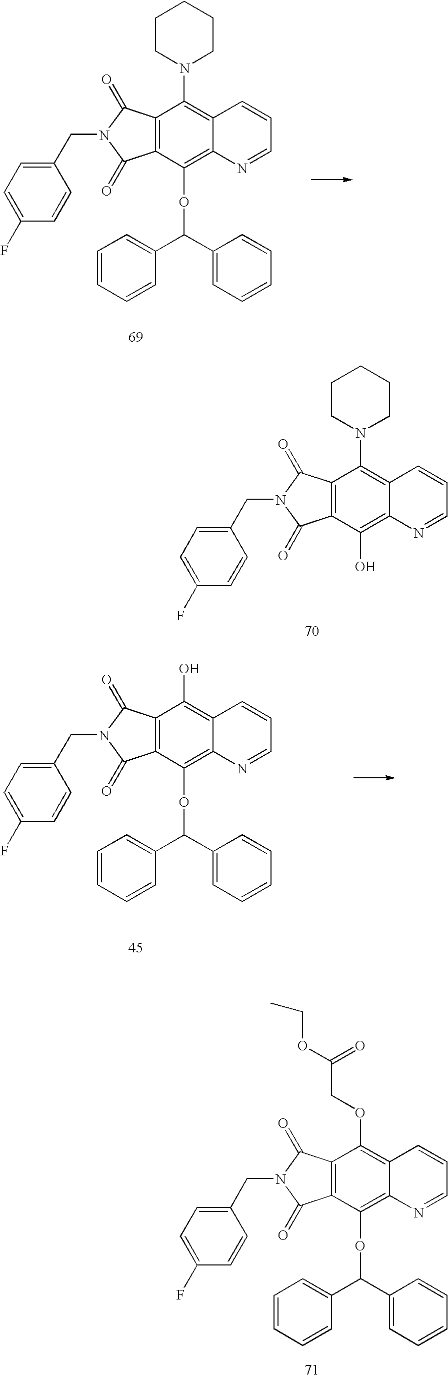

### Example 70

Tertiary amine 69 was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene, solidified with hexane and concentrated to give crude. Triturated with 1:1 diethylether/hexanes to give product 70 (0.0043 g, 0.0106 mmol, 72%.) ¹H NMR (CDCl₃) δ 8.96 (dd, 1H), 8.85 (d, 1H), 7.66 (dd, 1H), 7.5 (m, 2H), 7.04 (dd, 2H), 4.85 (s, 2H), 3.29 (br s, 4H), 1.77 (br s, 6H.) MS: 406 (M+1), 404 (M−1)

### Example 71

Monophenol 45 (0.03 g, 0.0595 mmol) was dissolved in 2 mL dry dimethylformamide. To this was added ethyl bromoacetate (0.0131 mL, 0.119 mmol) and freshly ground K₂CO₃ (0.025 g, 0.178 mmol.) Stirred at 50° C., for two hours until starting material consumed. Concentrated off some solvent, diluted with ethylacetate, washed with saturated NH₄Cl solution, concentrated organics to give crude product. Chromatographed (10 to 25% ethylacetate/hexanes) to give product 71 (0.0321 g, 0.054 mmol, 91%.) ¹H NMR (CDCl₃) δ 9.1 (dd, 1H), 8.96 (d, 1H), 7.9 (s, 1H), 7.62 (d, 4H), 7.445 (m, 2H), 7.27 (m, 7H), 7.059 (dd, 2H), 5.21 (s, 2H), 4.83 (s, 2H), 4.22 (q, 2H), 1.23 (t, 3H). MS: 591 (M+1).

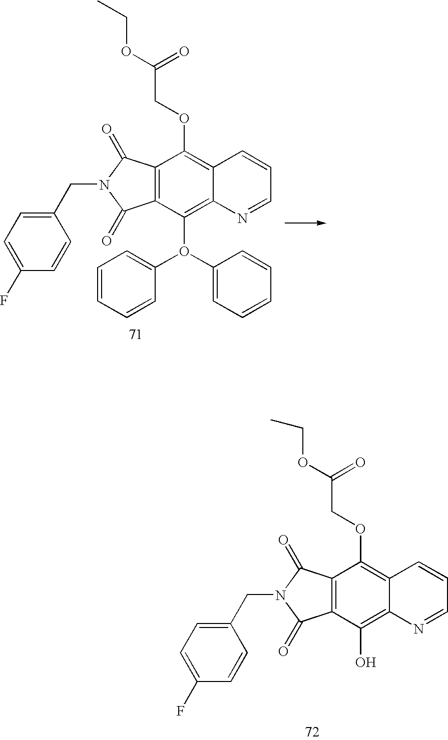

### Example 72

Ethyl ester 71 was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 72 (0.0209 g, 0.049 mmol, 91%.) ¹H NMR (CDCl₃) δ 9.0 (m, 2H), 7.7 (dd, 1H), 7.5 (dd, 2H), 7.04 (dd, 2H), 5.33 (s, 2H), 4.84 (s, 2H), 4.24 (q, 2H), 1.28 (t, 3H.) MS: 425 (M+1), 423 (M−1)

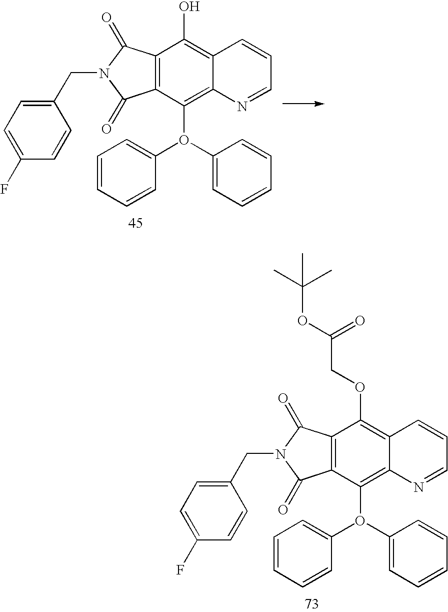

### Example 73

Monophenol 45 (0.03 g, 0.0595 mmol) was dissolved in 2 mL dry dimethylformamide. To this was added t-butyl bromoacetate (0.0175 mL, 0.119 mmol) and freshly ground K₂CO₃ (0.025 g, 0.178 mmol.) It was stirred at 50° C. for one hour until starting material was consumed. Concentrated off some solvent, diluted with ethylacetate, washed with saturated NH₄Cl solution, concentrated organics to give crude product. Chromatographed (10 to 15% ethylacetate/hexanes) to give product 73 (0.0309 g, 0.05 mmol, 84%.) ¹H NMR (CDCl₃) δ 9.09 (dd, 1H), 8.97 (d, 1H), 7.92 (s, 1H), 7.62 (d, 4H), 7.44 (m, 2H), 7.27 (m, 7H), 7.05 (dd, 2H), 5.12 (s, 2H), 4.83 (s, 2H), 1.38 (s, 9H.) MS: 619 (M+1)

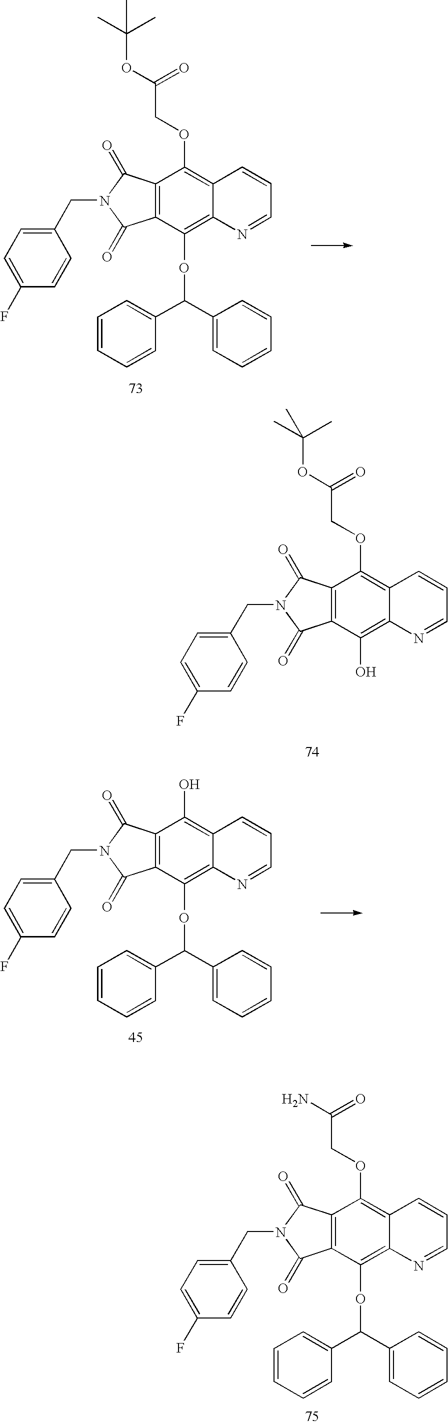

### Example 74

Tertiary Butyl ester 73 was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 74 (0.0189 g, 0.042 mmol, 84%.) ¹H NMR (CDCl₃) δ 9.05 (m, 2H), 7.72 (dd, 1H), 7.5 (dd, 2H), 7.04 (dd, 2H), 5.22 (s, 2H), 4.84 (s, 2H), 1.44 (s, 9H.) MS: 453 (M+1), 451 (M−1)

### Example 75

Monophenol 45 (0.04 g, 0.079 mmol) was dissolved in 1 mL dry dimethylformamide. To this was added 2-bromoacetamide (0.022 g, 0.158 mmol) and freshly ground K₂CO₃ (0.0345 g, 0.25 mmol.) Stirred at 60° C., for three hours until starting material nearly consumed. Concentrated off some solvent, diluted with ethylacetate, washed with saturated NaHCO₃ solution, concentrated organics to give crude product. Chromatographed (10 to 50% ethylacetate/hexanes) to give product 75 (0.0204 g, 0.0355 mmol, 46%.) ¹H NMR (CDCl₃) δ 9.15 (dd, 1H), 8.53 (d, 1H), 7.96 (s, 1H), 7.6 (m, 4H), 7.45 (dd, 2H), 7.27 (m, 7H), 7.06 (dd, 2H), 5.73 (br s, 1H), 4.84 (s, 2H), 4.77 (s, 2H.) MS: 562 (M+1)

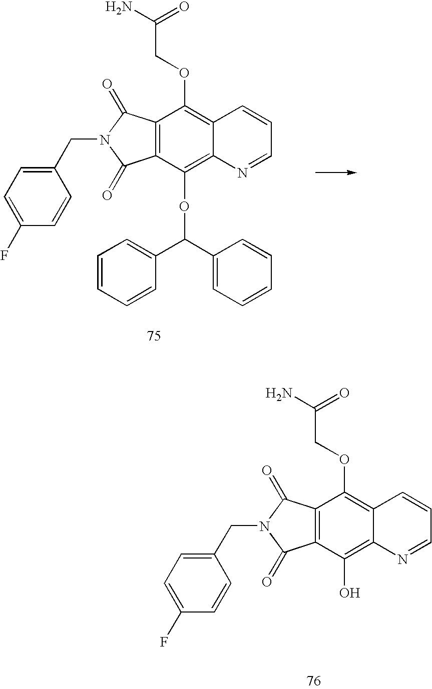

### Example 76

Amide 75 was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 76 (0.0095 g, 0.024 mmol, 67%.) ¹H NMR (CD₃SOCD₃) δ 9.08 (dd, 1H), 8.93 (d, 1H), 7.87 (dd, 1H), 7.73 (br s, 1H), 7.41 (dd, 2H), 7.19 (dd, 2H), 4.86 (s, 2H), 4.75 (s, 2H.) MS: 396 (M+1), 394 (M−1)

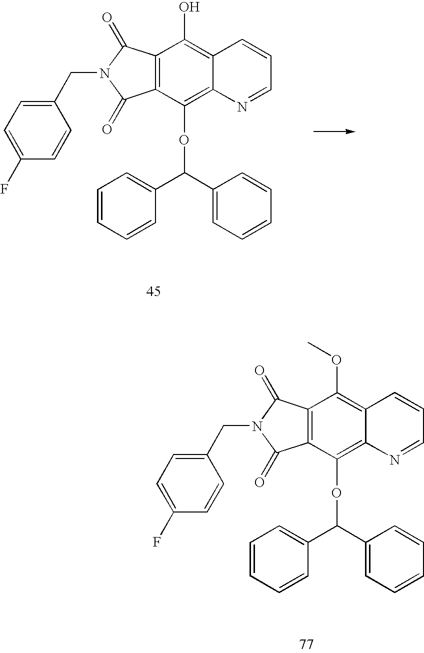

### Example 77

Monophenol 45 (2.9 g, 5.75 mmol) was dissolved in 20 mL dry dimethylformamide. To this was added methyl iodide (3.58 mL, 57.5 mmol) and freshly ground K₂CO₃ (3.17 g, 23 mmol.) Stirred at 40° C. for one hour, until starting material consumed. Diluted with dichloromethane, washed with saturated NH₄Cl solution, 2.5% LiCl solution, concentrated organics to give crude product. Chromatographed (15 to 55% ethylacetate/hexanes to give product 77 (2.54 g, 4.9 mmol, 85%.) ¹H NMR (CDCl₃) δ 9.1 (dd, 1H), 8.64 (dd, 1H), 7.91 (s, 1H), 7.62 (m, 5H), 7.46 (dd, 2H), 7.27 (m, 7H), 7.05 (dd, 2H), 4.84 (s, 2H), 4.28 (s, 3H.) MS: 519 (M+1)

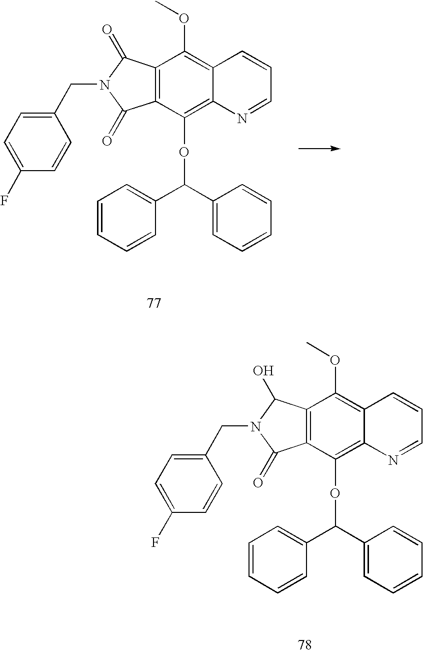

### Example 78

Methyl ether 77 was dissolved in 115 mL of dry tetrahydrofuran and 25 mL of dry methanol. To this was added three equivalents of a 0.5 M solution of NaBH4 (29.4 mL, 14.7 mmol) in 2-methoxyethyl ether. After 15 hrs at room temperature, concentrated off some solvent, diluted with dichloromethane, washed with 1M HCl solution with NaCl added, concentrated, chromatographed (15-66% ethylacetate/hexanes) to give oil. Triturated with hexane to give product 78 (1.3 g, 2.5 mmol, 68%.) ¹H NMR (CD₃SOCD₃) δ 9.08 (dd, 1H), 8.5 (d, 1H), 7.89 (s, 1H), 7.75 (d, 2H), 7.69 (dd, 1H), 7.63 (d, 2H), 7.42 (dd, 2H), 7.27 (m, 7H), 6.9 (d, 1H), 5.92 (dd, 1H), 4.97 (d J=15 Hz, 1H), 4.45 (d J=15 Hz, 1H), 4.04 (s, 3H.) MS: 521 (M+1)

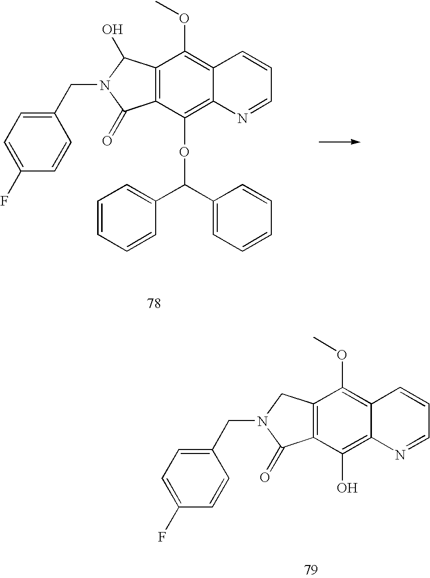

### Example 79

Aminal 78 was dissolved in 15 mL of dichloromethane. To this was added 2 mL of triethylsilane and 1 mL of trifluoroacetic acid. Stirred at room temperature and after ten minutes complete by TLC. Concentrated off volatiles, azeotroped with toluene to give crude. Triturated with 1:1 diethylether/hexanes to give reduced product. Dissolved in 30 mL of dichloromethane and cooled to 0° C. To this was added 4 mL of triethylsilane and trimethylsilyltriflate (1.36 mL, 7.5 mmol.) Stirred vigorously for three minutes, then concentrated off volatiles, diluted with dichloromethane, washed quickly with saturated NaHCO₃ solution, concentrated organics to give crude product 79. Triturated with 1:1 diethylether/hexanes to give product (0.806 g, 2.38 mmol, 95% for two steps.) ¹H NMR (CDCl₃) δ 8.96 (dd, 1H), 8.50 (d, 1H), 7.56 (dd, 1H), 7.37 (dd, 2H), 7.09 (dd, 2H), 4.78 (s, 2H), 4.51 (s, 2H), 3.98 (s, 3H). MS: 339 (M+1), 337 (M−1).

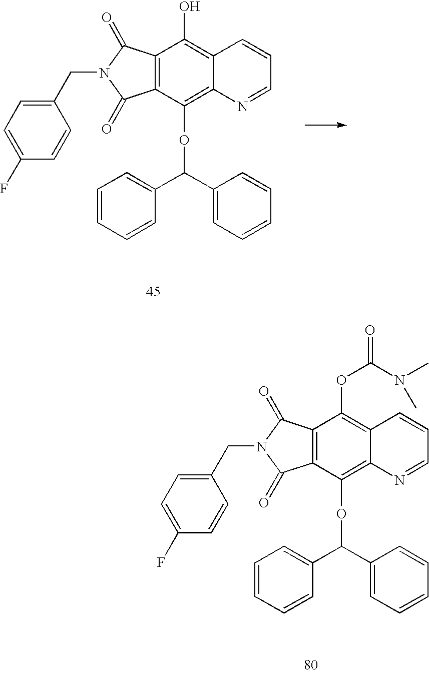

### Example 80

Monophenol 45 (0.02 g, 0.0396 mmol) was dissolved in 1 mL dry dichloromethane. To this was added at 0° C. triethylamine (0.0165 mL, 0.1188 mmol) and dimethylcarbamoyl chloride (0.0054 mL, 0.0594 mmol). Catalytic amount of DMAP was also added. Stirred at room temperature overnight. Dilute with dichloromethane, washed with saturated NaHCO₃ solution and saturated NH₄Cl solution, concentrated to give crude. Triturated with 1:1 diethylether/hexanes and chromatographed (10% methanol/45% ethylacetate/45% hexanes) to give product 80 (0.012 g, 0.0198 mmol, 50%.) ¹H NMR (CDCl₃) δ 9.12 (s, 1H), 8.4 (d, 1H), 7.97 (s, 1H), 7.62 (d, 4H), 7.43 (dd, 2H), 7.27 (m, 7H), 7.05 (dd, 2H), 4.81 (s, 2H), 3.26 (s, 3H), 3.09 (s, 3H.) MS: 576 (M+1)

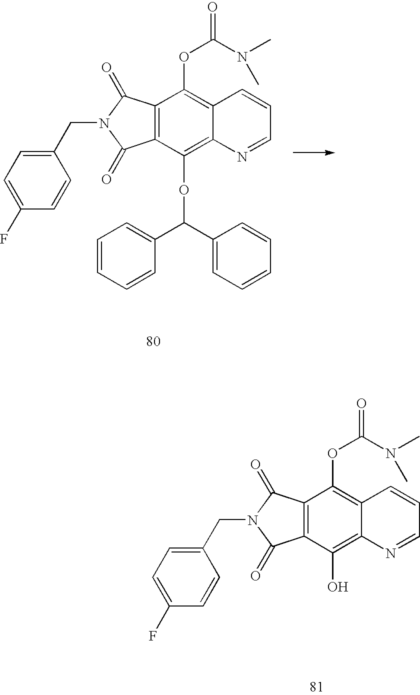

### Example 81

Carbamate 80 (0.012 g, 0.0198 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 81 (0.0054 g, 0.013 mmol, 67%.) ¹H NMR (CDCl₃) δ 8.98 (s, 1H), 8.49 (d, 1H), 7.7 (dd, 1H), 7.46 (dd, 2H), 7.03 (dd, 2H), 4.83 (s, 2H), 3.31 (s, 3H), 3.12 (s, 3H). MS: 410 (M+1), 408 (M−1).

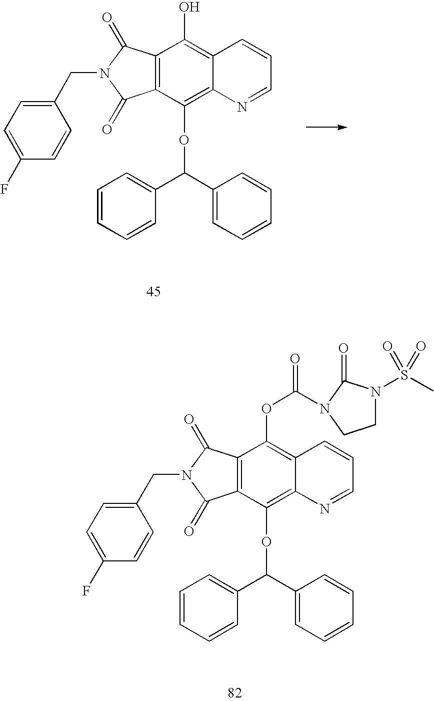

### Example 82

Monophenol 45 (0.035 g, 0.0694 mmol) was dissolved in 1 mL dry dichloroethane. To this was added triethylamine (0.038 mL, 0.277 mmol) and 3-chlorocarbonyl-1-methanesulfonyl-2-imidazolidinone (0.0314 g, 0.1388 mmol Stirred at room temperature for five minutes. Dilute with dichloromethane, washed with saturated NaHCO₃ solution and saturated NH₄Cl solution, dried (MgSO₄), concentrated to give crude. Chromatographed (10% methanol/45% ethylacetate/45% hexanes) to give product 82 (0.036 g, 0.0518 mmol, 75%.) ¹H NMR (CDCl₃) 9.16 (dd, 1H), 8.49 (dd, 1H), 8.00 (s, 1H), 7.66 (dd, 1H), 7.61 (d, 4H), 7.40 (dd, 2H), 7.27 (m, 6H), 7.05 (dd, 2H), 4.81 (s, 2H), 4.2 (dd, 2H), 4.08 (dd, 2H), 3.92 (s, 3H). MS: 695 (M+1).

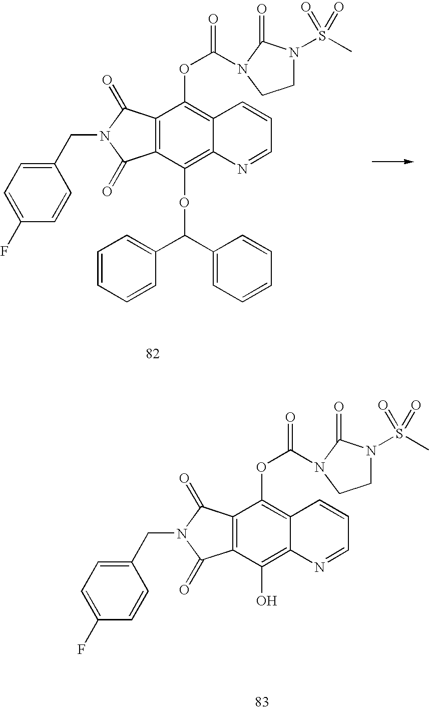

### Example 83

Carbamate 82 (0.036 g, 0.0518 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 83 (0.025 g, 0.047 mmol, 91%.) ¹H NMR (CDCl₃) δ 9.04 (d, 1H), 8.58 (d, 1H), 7.75 (dd, 1H), 7.43 (dd, 2H), 7.04 (dd, 2H), 4.82 (s, 2H), 4.22 (dd, 2H), 4.10 (dd, 2H). MS: 529 (M+1), 527 (M−1).

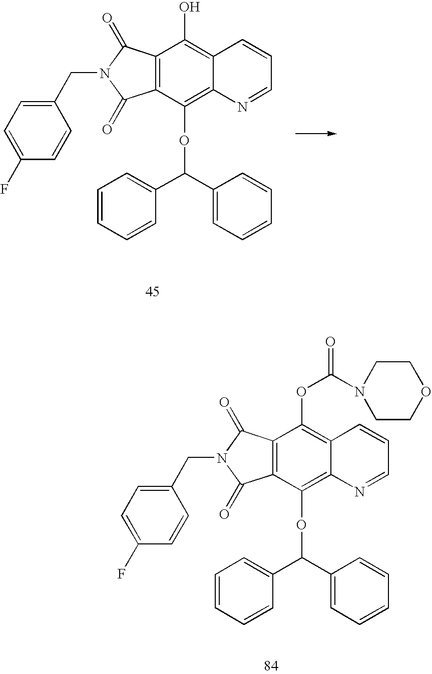

### Example 84

Monophenol 45 (0.045 g, 0.089 mmol) was dissolved in 1 mL dry dichloroethane. To this was added triethylamine (0.049 mL, 0.356 mmol) and 4-morpholine carbonyl chloride (0.0207 mL, 0.178 mmol.) Stirred at room temperature for 1.5 hours. Dilute with dichloromethane, washed with saturated NaHCO₃, concentrated to give crude. Chromatographed (15% to 60% ethylacetate/hexanes) to give product 84 (0.039 g, 0.063 mmol, 71%.) ¹H NMR (CDCl₃) 9.13 (dd, 1H), 8.40 (d, 1H), 7.98 (s, 1H), 7.62 (dd, 4H), 7.4 (dd, 2H), 7.27 (m, 7H), 7.05 (dd, 2H), 4.81 (s, 2H), 3.84 (br s, 6H), 3.62 (br s, 2H.) MS: 618 (M+1)

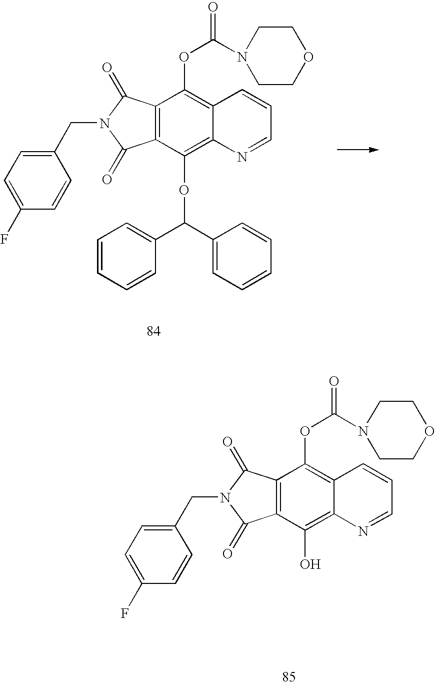

### Example 85

Carbamate 84 (0.039 g, 0.063 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated with 1:1 diethylether/hexanes to give product 85 (0.014 g, 0.032 mmol, 51%.) ¹H NMR (CDCl₃) δ 9.0 (d, 1H), 8.48 (d, 1H), 7.72 (dd, 1H), 7.49 (dd, 2H), 7.04 (dd, 2H), 4.83 (s, 2H), 3.88 (br s, 6H), 3.66 (br s, 2H.) MS: 452 (M+1), 450 (M−1)

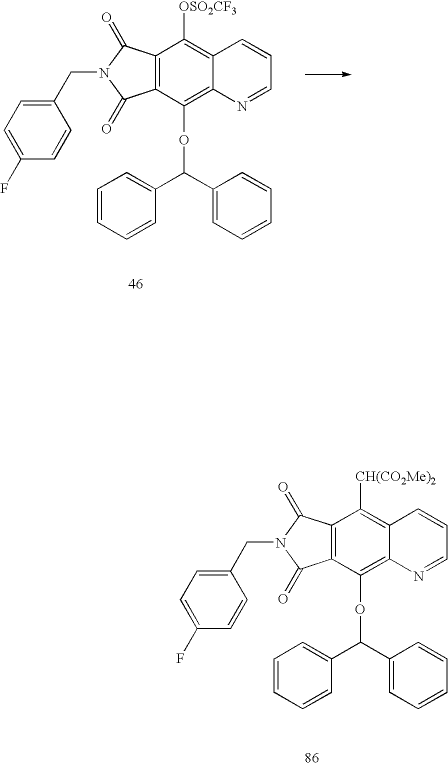

### Example 86

Triflate 46 in benzene concentrated to give (0.048 g, 0.075 mmol) and dissolved in 1 mL dry tetrahydrofuran. To this was added freshly ground K₂CO₃ (0.069, 0.5 mmol) and dimethylmalonate (0.017 mL, 0.15 mmol) and stirred at 50° C. After 15 hours, starting material consumed, concentrated to give oil. Chromatographed (5% to 30% ethylacetate/hexanes) to give product 86 (0.012 g, 0.0195 mmol, 26%.) ¹H NMR (CDCl₃) δ 9.09 (d, 1H), 8.51 (d, 1H), 8.12 (s, 1H), 7.65 (d, 4H), 7.57 (dd, 1H), 7.48 (dd, 2H), 7.27 (m, 6H), 7.07 (dd, 2H), 4.85 (s, 2H), 3.72 (6H.) MS: 619 (M+1)

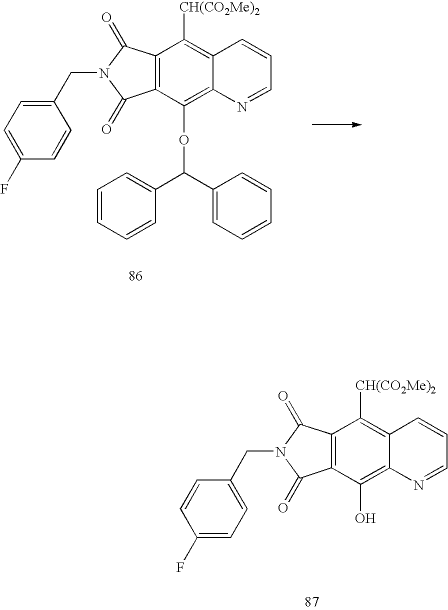

### Example 87

Di-ester 86 (0.008 g, 0.0129 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 87 (0.0022 g, 0.0049 mmol, 38%.) ¹H NMR (CDCl₃) δ 8.95 (d, 1H), 8.60 (d, 1H), 7.70 (dd, 1H), 7.55 (dd, 2H), 7.05 (dd, 2H), 4.87 (s, 2H), 3.76 (s, 6H.) MS: 453 (M+1), 451 (M−1)

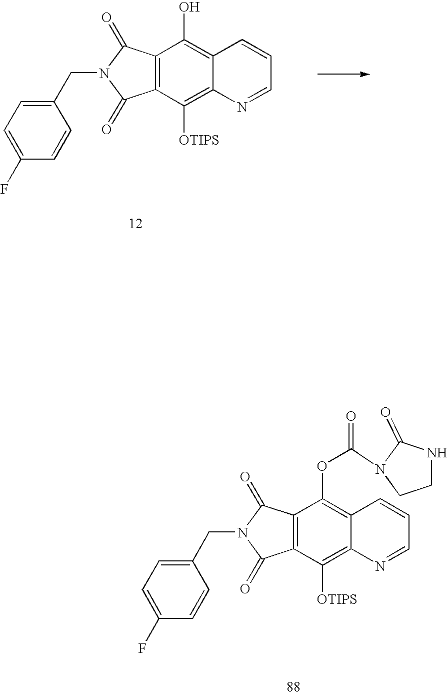

### Example 88

Mono-phenol 12 (0.03 g, 0.06 mmol) was dissolved in 1 mL of dichloroethane. To this was added triethylamine (0.033 mL, 0.24 mmol) and 2-oxo-1-imidazolidinecarbonyl chloride (0.0178 g, 0.12 mmol.) Catalytic amount of DMAP added, and stirred at room temperature for three hours. Diluted with dichloromethane, washed with saturated NH₄Cl solution, concentrated to give crude. Chromatographed (10% ethylacetate/hexane to 10% methanol/45% ethylacetate/45% hexanes) to give product 88 (0.0247 g, 0.0395 mmol, 68%.) ¹H NMR (CDCl₃) δ 8.96 (s, 1H), 8.53 (d, 1H), 7.63 (dd, 1H), 7.43 (dd, 2H), 7.03 (dd, 2H), 4.81 (s, 2H), 4.25 (dd, 2H), 3.69 (dd, 2H), 1.55 (m, 3H), 1.14 (d, 18H.) MS: 607 (M+1)

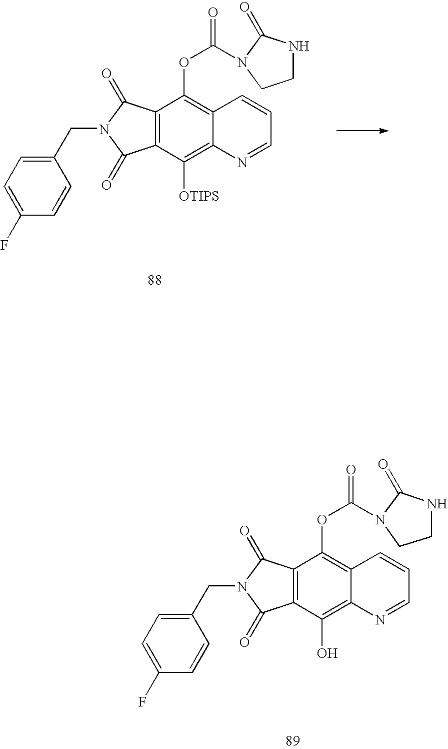

### Example 89

Urea 88 (0.024 g, 0.0395 mmol) was dissolved in 1 mL of dry dichloromethane. To this was added ten equivalents (0.03 mL, 0.395 mmol) of trifluoroacetic acid. Stirred at room temperature, for fifteen hours. Concentrated off volatiles, azeotroped with toluene (2×), concentrated to give crude. Crude product triturated with 1:1 diethylether/hexanes to give product 89 (0.0119 g, 0.026 mmol, 67%.) ¹H NMR (CDCl₃) δ 9.00 (d, 1H), 8.58 (d, 1H), 7.73 (dd, 1H), 7.47 (dd, 2H), 7.03 (dd, 2H), 4.83 (s, 2H), 4.28 (dd, 2H), 3.70 (dd, 2H.) MS: 451 (M+1), 449 (M−1)

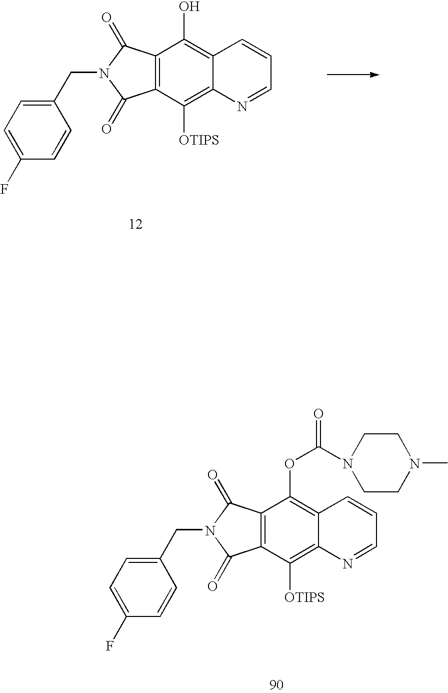

### Example 90

Mono-phenol 12 (0.04 g, 0.08 mmol) was dissolved in 1.5 mL dry tetrahydrofuran. To this was added triethylamine (0.0445 mL, 0.32 mmol) and bispentafluorophenyl carbonate (0.063 g, 0.16 mmol) and catalytic dimethylaminopyridine. Stirred at room temperature. After three hours, added methyl piperazine (0.04 mL, 0.36 mmol.) After two hours TLC indicated product formed however TIPSCl was removed. Diluted with dichloromethane, washed with saturated NH₄Cl solution, concentrated organics to give crude. Dissolved in 1.5 mL dichloroethane, added triethylamine (0.11 mL, 0.8 mmol) and TIPSCl (0.085 mL, 0.4 mmol) and stirred at 50° C. Stirred for four hours until starting material was consumed. Diluted with dichloromethane, washed with saturated brine, concentrated organics to give crude. Chromatographed (50% ethylacetate/hexanes to 20% methanol/60% ethylacetate/20% hexanes) to give product 90 (0.027 g, 0.0435 mmol, 54% for two steps.) ¹H NMR (CDCl₃) δ 9.05 (d, 1H), 8.60 (d, 1H), 7.61 (dd, 1H), 7.41 (dd, 2H), 7.03 (dd, 2H), 4.81 (s, 2H), 3.71 (br m, 8H), 2.43 (s, 3H), 1.60 (m, 3H), 1.15 (d, 18H.) MS: 621 (M+1)

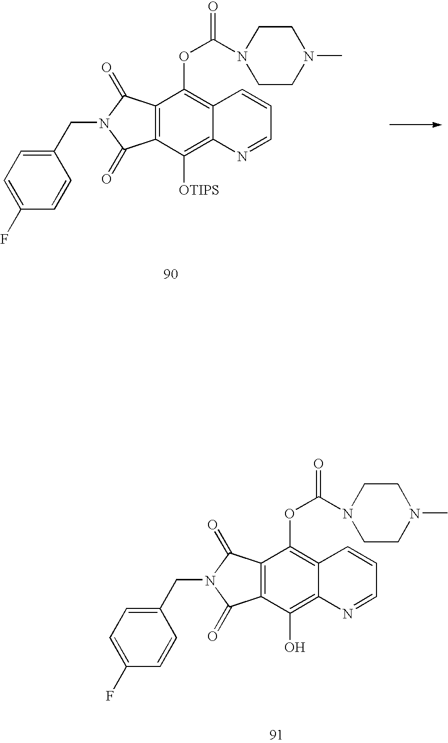

### Example 91

Mono-carbamate 90 (0.027 g, 0.0435 mmol) was dissolved in 1 mL of dichloromethane. To this was added trifluoroacetic acid (0.067 mL, 0.87 mmol) and stirred at room temperature. After twenty hours, concentrated off volatiles, azeotroped with toluene (2×), concentrated to give crude. Triturate with 1:1 diethylether/hexanes to give product 91 (0.0177 g, 0.038 mmol, 87%.) ¹H NMR (CD₃SOCD₃) δ 9.09 (s, 1H), 8.71 (d, 1H), 7.67 (dd, 1H), 7.42 (dd, 2H), 7.07 (dd, 2H), 4.81 (s, 2H), 3.45 (br m, 8H), 2.90 (s, 3H.) MS: 465 (M+1), 463 (M−1)

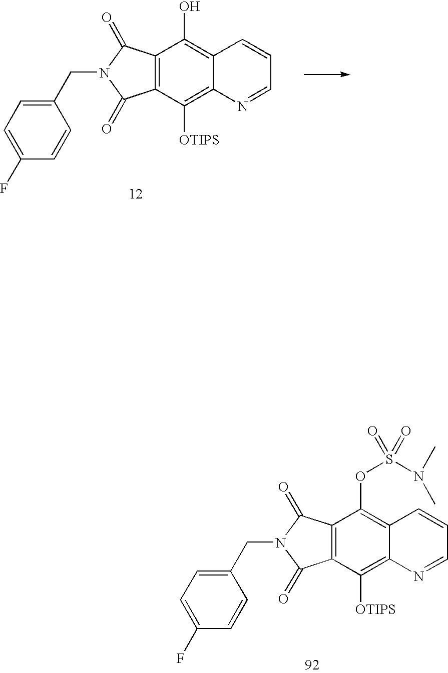

### Example 92

Mono-phenol 12 (0.04 g, 0.08 mmol) was dissolved in 1.5 mL dichloromethane. To this was added triethylamine (0.044 mL, 0.32 mmol), dimethylsulfamoyl chloride (0.017 mL, 0.16 mmol) and catalytic dimethylaminopyridine. Stirred at room temperature for 30 minutes. Diluted with dichloromethane, washed with saturated NH4Cl solution, concentrated organics to give crude. Chromatographed (25% ethylacetate/hexanes) to give product 92 (0.017 g, 0.02828 mmol, 35%.) ¹H NMR (CDCl₃) δ 8.95 (d, 1H), 8.79 (d, 1H), 7.66 (dd, 1H), 7.45 (dd, 2H), 7.03 (dd, 2H), 4.84 (s, 2H), 3.24 (s, 6H), 1.55 (m, 3H), 1.14 (d, 18H.) MS: 602 (M+1)

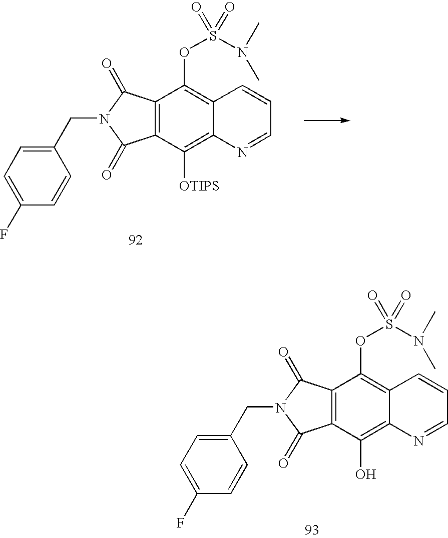

### Example 93

Mono-carbamate 92 (0.017 g, 0.02828 mmol) was dissolved in 1 mL of dichloromethane. To this was added trifluoroacetic acid (0.044 mL, 0.5657 mmol) and stirred at room temperature. After twenty hours, concentrated off volatiles, azeotroped with toluene (2×), concentrated to give crude. Triturate with 1:1 diethylether/hexanes to give product 93 (0.0081 g, 0.018 mmol, 64%.) ¹H NMR (CDCl₃) δ 9.00 (d, 1H), (8.84 (d, 1H), 7.76 (dd, 1H), 7.49 (dd, 2H), 7.03 (dd, 2H), 4.86 (s, 2H), 3.24 (s, 6H.) MS: 446 (M+1), 444 (M−1)

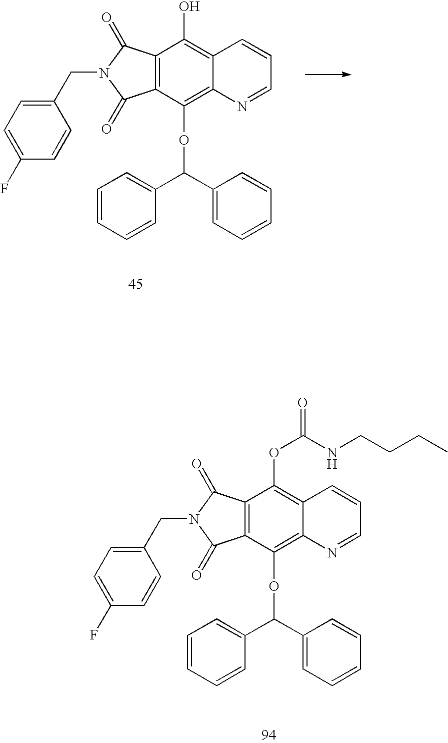

### Example 94

Mono-phenol 45 (0.04 g, 0.08 mmol) was dissolved in 1.5 mL tetrahydrofuran. To this was added diisopropylethylamine (0.052 mL, 0.3 mmol), bis-pentafluorophenyl carbonate (0.047 g, 0.119 mmol) and catalytic dimethylaminopyridine. Stirred at room temperature. After 75 minutes, cooled to 0° C., n-butylamine (0.079 mL, 0.08 mmol) added. Stirred for 1.5 hours, then diluted with dichloromethane, washed with saturated brine, 1 M HCl, concentrated organics to give crude. Chromatographed (25% ethylacetate/hexanes to give product 94 (0.0028 g, 0.0048 mmol, 6%.) ¹H NMR (CDCl₃) δ 9.12 (d, 1H), 8.41 (d, 1H), 7.98 (s, 1H), 7.61 (d, 4H), 7.43 (dd, 2H), 7.27 (m, 7H), 7.043 (dd, 2H), 5.37 (m, 1H), 4.82 (s, 2H), 3.35 (q, 2H), 1.67 (m, 2H), 1.49 (m, 2H), 1.01 (t, 3H.) MS: 604 (M+1)

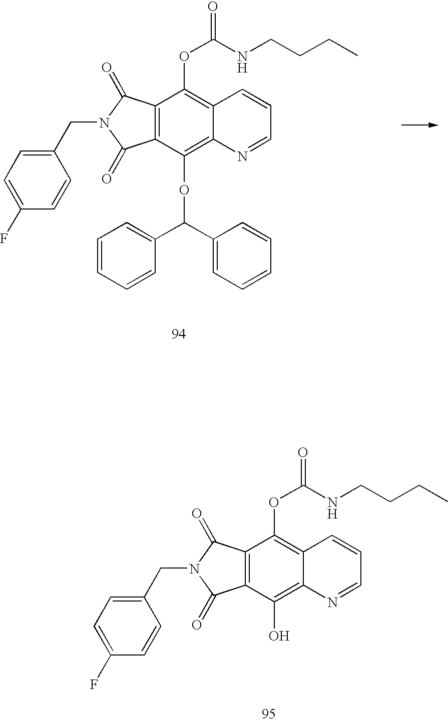

### Example 95

Carbamate 94 (0.006 g, 0.0099 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude which was triturated twice with 1:1 diethylether/hexanes to give product 95 (0.0014 g, 0.003 mmol, 32%.) ¹H NMR (CDCl₃) δ 8.98 (s, 1H), 8.49 (d, 1H), 7.68 (dd, 1H), 7.47 (dd, 2H), 7.03 (dd, 2H), 5.40 (m, 1H), 4.83 (s, 2H), 3.38 (q, 2H), 3.15 (m, 2H), 1.49 (m, 2H), 1.03 (t, 3H.) MS: 438 (M+1), 436 (M−1)

### Example 96

Monophenol 45 (0.05 g, 0.099 mmol) was dissolved in 0.5 mL of dichloromethane. To this was added triethylamine (0.03 mL, 0.2 mmol) and pyrrolidine carbonyl chloride (0.0214 mL, 0.2 mmol.) Stirred at 30° C. for fifteen hours. Diluted with dichloromethane, washed with 1 M HCl solution, concentrated organics to give crude. Chromatographed (20% to 50% ethylacetate/hexanes) to give product 96 (0.033 g, 0.0555 mmol, 57%.) ¹H NMR (CDCl₃) δ 9.11 (dd, 1H), 8.45 (d, 1H), 7.97 (s, 1H), 7.62 (d, 5H), 7.40 (dd, 2H), 7.27 (m, 6H), 7.05 (dd, 2H), 4.81 (s, 2H), 3.75 (dd, 2H), 3.54 (dd, 2H), 2.05 (m, 4H.) MS: 602 (M+1)

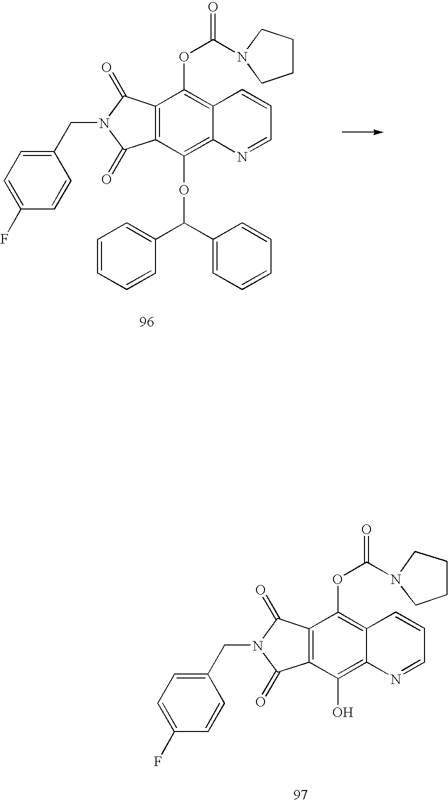

### Example 97

Carbamate 96 (0.033 g, 0.055 mmol) was dissolved in 0.5 mL of dichloromethane. Triethylsilane (0.2 mL) and of trifluoroacetic acid (0.1 mL) were added. The mixture was stirred at room temperature and was complete after ten minutes by TLC. The mixture was concentrated in vacuo and azeotroped with toluene to give a crude residue which was triturated twice with 1:1 diethylether/hexanes to give product 97 (0.0123 g, 0.028 mmol, 51%). ¹H NMR (CDCl₃) δ 8.98 (d, 1H), 8.51 (d, 1H), 7.70 (dd, 1H), 7.46 (dd, 2H), 7.03 (dd, 2H), 4.82 (s, 2H), 3.81 (dd, 2H), 3.57 (dd, 2H), 2.09 (m, 4H.) MS: 436 (M+1), 434 (M−1)

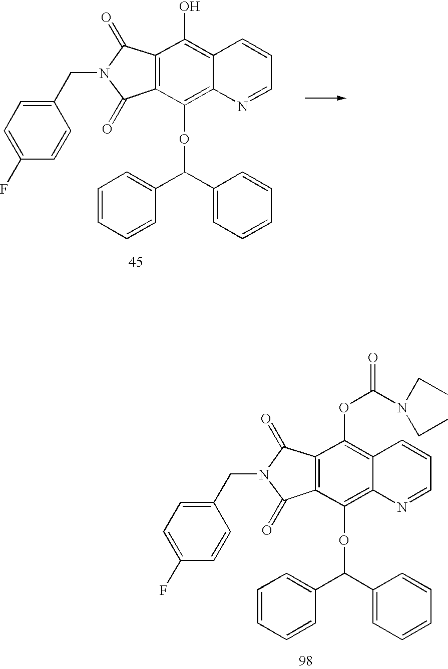

### Example 98

Monophenol 45 (0.03 g, 0.06 mmol) was dissolved in 1.5 mL of dichloromethane. Triethylamine (0.033 mL, 0.238 mmol) and diethylcarbamoyl chloride (0.015 mL, 0.119 mmol) were added. The mixture was stirred at 60° C. for five hours. The mixture was diluted with dichloromethane, washed with 1 M HCl solution, and concentrated to give crude product. The crude product was chromatographed (20% to 50% ethylacetate/hexanes) to give product 98 (0.0237 g, 0.040 mmol, 66%.) ¹H NMR (CDCl₃) δ 9.12 (s, 1H), 8.34 (d, 1H), 7.97 (s, 1H), 7.63 (d, 4H), 7.40 (dd, 2H), 7.27 (m, 7H), 7.01 (dd, 2H), 4.81 (s, 2H), 3.61 (dd, 2H), 3.50 (q, 2H), 1.41 (t, 3H), 1.37 (t, 3H.) MS: 604 (M+1)

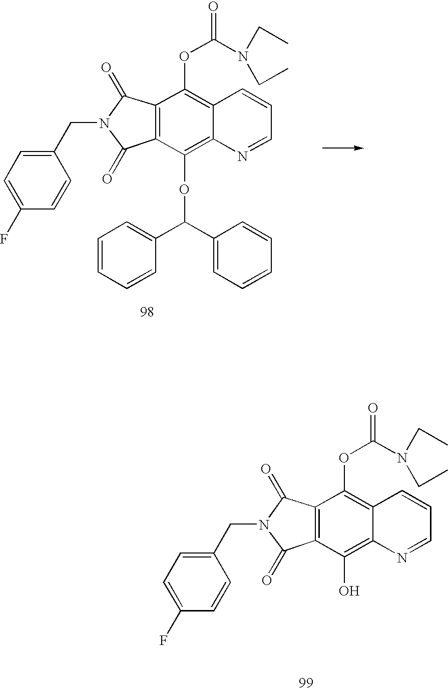

### Example 99

Carbamate 98 (0.023 g, 0.04 mmol) was dissolved in 0.5 mL of dichloromethane. Triethylsilane (0.2 mL) and trifluoroacetic acid (0.1 mL) were added. The mixture was stirred at room temperature and after ten minutes was complete by TLC. Concentrated off volatiles, azeotroped with toluene to give crude. Triturated twice with 1:1 diethylether/hexanes to give product 99 (0.01 g, 0.024 mmol, 60%.) ¹H NMR (CDCl₃) δ 8.98 (d, 1H), 8.45 (d, 1H), 7.70 (dd, 1H), 7.48 (dd, 2H), 7.03 (dd, 2H), 4.82 (s, 2H), 3.67 (q, 2H), 3.48 (q, 2H), 1.46 (t, 3H), 1.32 (t, 3H.) MS: 438 (M+1), 436 (M−1)

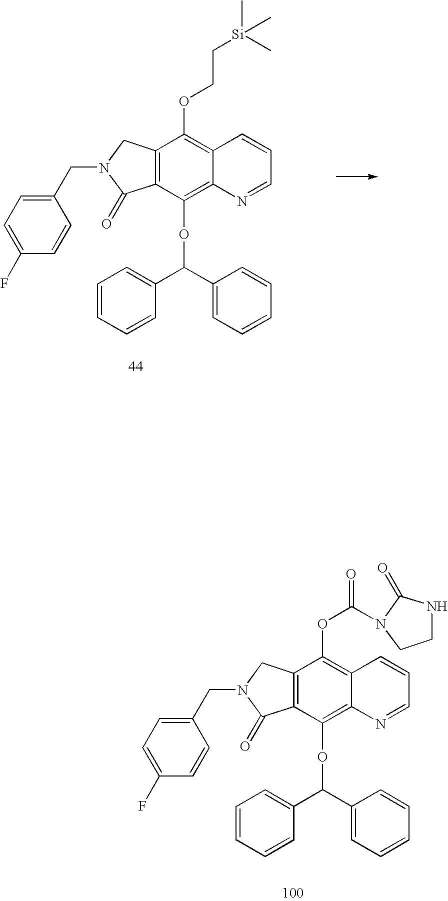

### Example 100

Trimethylsilyl ether 44 (0.022 g, 0.0373 mmol) was dissolved in 0.5 mL dry tetrahydrofuran. To this was added triethylamine (0.031 mL, 0.2238 mmol) and 1 M tetrabutylammonium fluoride solution in tetrahydrofuran (0.0559 mL, 0.0559 mmol.) Stirred at room temperature 10 minutes until starting material consumed. Then added catalytic amount of dimethylaminopyridine and 2-oxo-1-imidazolidinecarbonyl chloride (0.022 g, 0.1492 mmol.) Stirred at room temperature for three hours, then diluted with dichloromethane, washed with 1M HCl solution, saturated NaHCO₃, saturated brine, concentrated to give crude. Chromatographed (50% ethylacetate/hexanes to 1:1:1 methanol, ethylacetate, hexanes) to give product 100 (0.0197 g, 0.031 mmol, 88%.) ¹H NMR (CDCl₃) δ 9.04 (dd, 1H), 8.31 (d, 1H), 8.02 (s, 1H), 7.73 (d, 4H), 7.53 (dd, 1H), 7.27 (m, 6H), 7.04 (dd, 2H), 5.00 (s, 1H) 4.80 (s, 2H), 4.10 (dd, 2H), 3.64 (dd, 2H.) MS: 603 (M+1)

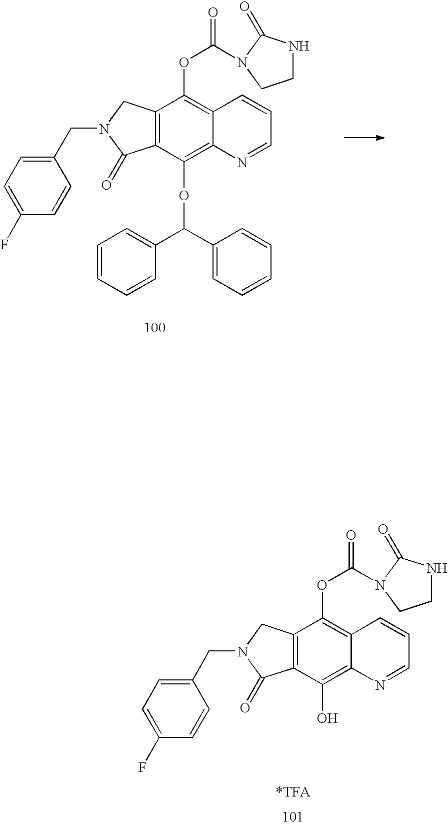

### Example 101

Carbamate 100 (0.019 g, 0.031 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 101 (0.006 g, 0.011 mmol, 35%.) ¹H NMR (CD₃SOCD₃) δ 8.98 (s, 1H), 8.48 (d, 1H), 7.77 (dd, 1H), 7.72 (s, 1H), 7.36 (dd, 2H), 7.22 (dd, 2H), 4.70 (s, 2H), 4.37 (s, 2H), 4.03 (dd, 2H), 3.41 (dd, 2H.) ¹⁹F NMR: −74.6 MS: 437 (M+1), 435 (M−1)

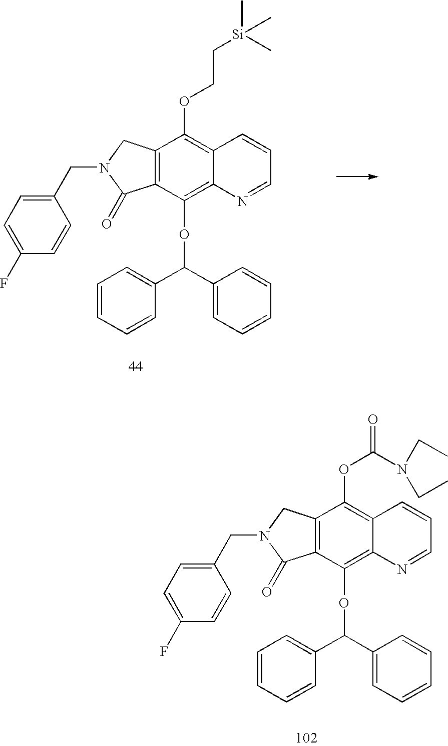

### Example 102

Trimethylsilylethyl ether 44 (0.03 g, 0.0508 mmol) was dissolved in 0.5 mL dry tetrahydrofuran. Triethylamine (0.042 mL, 0.3048 mmol) and 1 M tetrabutylammonium fluoride solution in tetrahydrofuran (0.1016 mL, 0.1016 mmol) were added and stirred at room temperature for 10 minutes until starting material was consumed. A catalytic amount of dimethylaminopyridine was added, followed by diethylcarbamoyl chloride (0.026 mL, 0.2032 mmol). The mixture was stirred at room temperature for four hours, then diluted with dichloromethane, washed with 1M HCl solution, saturated NaHCO₃, saturated brine, and concentrated to the crude product. Chromatographed (25% to 50% ethylacetate/hexanes) to give product 102 (0.014 g, 0.024 mmol, 47%.) ¹H NMR (CDCl₃) δ 9.04 (s, 1H), 8.11 (d, 1H), 8.03 (s, 1H), 7.76 (d, 4H), 7.51 (dd, 1H), 7.27 (m, 8H), 7.08 (dd, 2H), 4.80 (s, 2H), 4.21 (s, 2H), 3.53 (q, 2H), 3.40 (q, 2H), 1.33 (t, 3H), 1.23 (t, 3H.) MS: 590 (M+1)

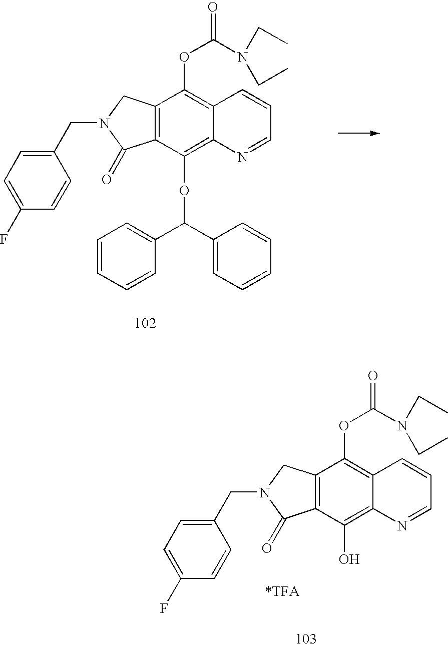

### Example 103

Carbamate 102 (0.01 g, 0.0169 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 103 (0.0073 g, 0.014 mmol, 80%.) ¹H NMR (CDCl₃) δ 9.01 (s, 1H), 8.23 (d, 1H), 7.60 (dd, 1H), 7.33 (dd, 2H), 7.09 (dd, 2H), 4.77 (s, 2H), 4.37 (s, 2H), 3.56 (q, 2H), 3.43 (q, 2H), 1.37 (t, 3H), 1.26 (t, 3H.) ¹⁹F NMR: −76.2 MS: 424 (M+1), 422 (M−1)

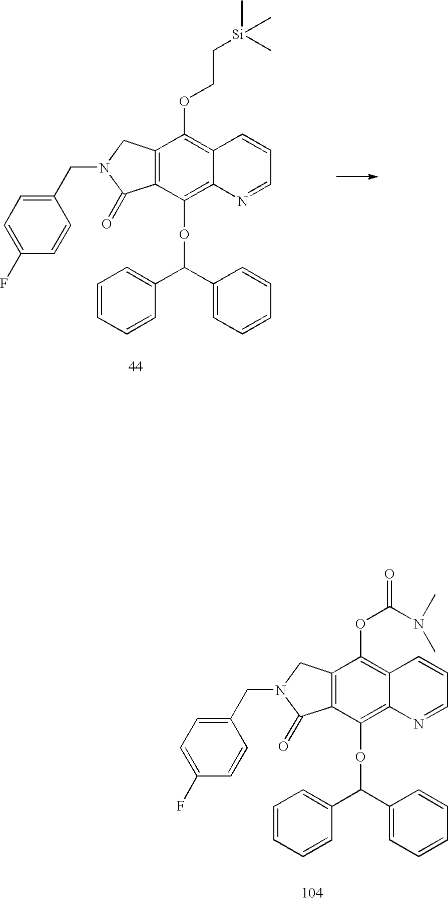

### Example 104

Trimethylsilylethyl ether 44 (0.03 g, 0.0508 mmol) was dissolved in 0.5 mL dry tetrahydrofuran. To this was added triethylamine (0.042 mL, 0.3048 mmol) and 1 M tetrabutylammonium fluoride solution in tetrahydrofuran (0.1016 mL, 0.1016 mmol.) Stirred at room temperature 10 minutes until starting material consumed. Then added catalytic amount of dimethylaminopyridine and dimethylcarbamoyl chloride (0.0187 mL, 0.2032 mmol.) Stirred at room temperature for six hours, then diluted with dichloromethane, washed with 1M HCl solution, saturated NaHCO₃, saturated brine, concentrated to give crude. Chromatographed (20% to 50% ethylacetate/hexanes) to give product 104 (0.014 g, 0.024 mmol, 48%.) ¹H NMR (CDCl₃) δ 9.04 (d, 1H), 8.14 (d, 1H), 8.03 (s, 1H), 7.75 (d, 4H), 7.51 (dd, 1H), 7.27 (m, 8H), 7.16 (dd, 2H), 4.80 (s, 2H0, 4.23 (s, 2H), 3.19 (s, 3H), 3.02 (s, 3H.) MS: 562 (M+1)

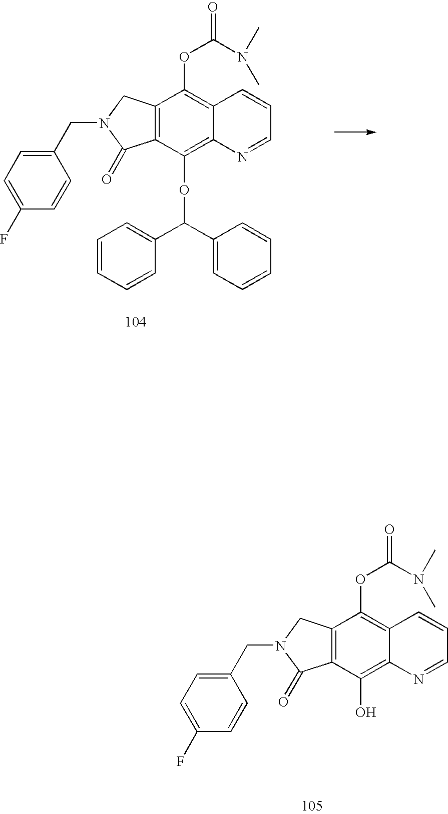

### Example 105

Carbamate 104 (0.012 g, 0.021 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 105 (0.0068 g, 0.017 mmol, 82%.) ¹H NMR (CDCl₃) δ 8.96 (s, 1H), 8.25 (d, 1H), 7.59 (dd, 1H), 7.36 (dd, 2H), 7.09 (dd, 2H), 4.77 (s, 2H), 4.38 (s, 2H), 3.24 (s, 3H), 3.06 (s, 3H.) MS: 396 (M+1), 394 (M−1)

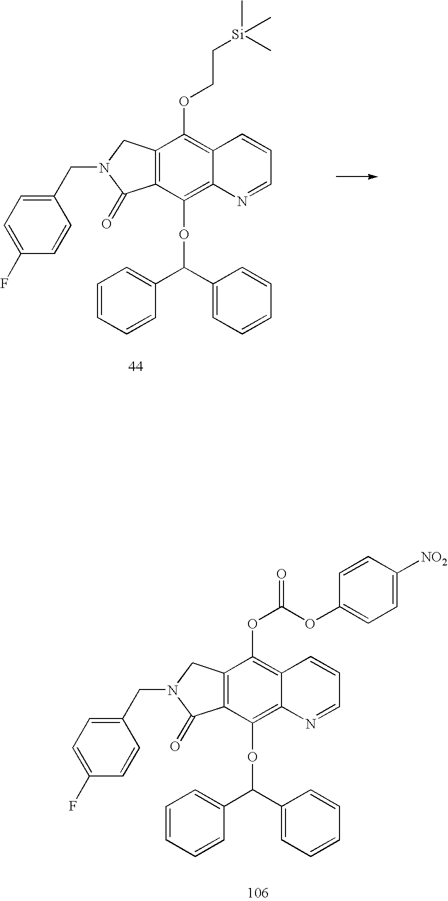

### Example 106

Trimethylsilylethyl ether 44 (0.03 g, 0.0508 mmol) was dissolved in 0.5 mL dry tetrahydrofuran. To this was added triethylamine (0.0282 mL, 0.2032 mmol) and 1 M tetrabutylammonium fluoride solution in tetrahydrofuran (0.076 mL, 0.076 mmol.) Stirred at room temperature 10 minutes until starting material consumed. After fifteen minutes, diluted with dichloromethane, washed with washed with 1M HCl solution, saturated NaHCO₃, saturated brine, concentrated to give crude. Diluted in 1 mL dichloromethane. To this was added triethylamine (0.028 mL, 0.2032 mmol), para-nitrochloroformate (0.02 g, 0.1016 mmol) and catalytic dimethylaminopyridine. Stirred at room temperature for 30 minutes, then diluted with dichloromethane, washed with saturated NH₄Cl solution, concentrated organics to give crude. Chromatographed (50% ethylacetate/hexanes) to give product 106 (0.009 g, 0.0137 mmol, 27%) ¹H NMR (CDCl₃) δ 9.10 (s, 1H), 8.16 (d, 2H), 8.10 (s, 1H), 7.71 (d, 4H), 7.53 (dd, 1H), 7.27 (m, 9H), 7.09 (dd, 2H), 6.93 (d, 2H), 4.79 (s, 2H), 4.23 (s, 2H.) MS: 656 (M+1)

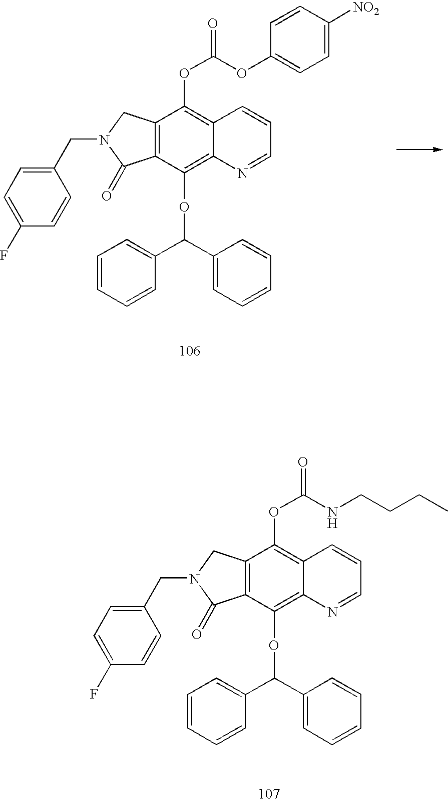

### Example 107

Carbonate 106 (0.009 g, 0.0137 mmol) was dissolved in 0.5 mL dichloromethane. To this was added triethylamine (0.0282 mL, 0.2032 mmol) and n-butylamine (0.01 mL, 0.1016 mmol) and stirred at room temperature. After 15 minutes, starting material consumed. Diluted with dichloromethane, washed with 1M HCl solution, saturated brine, concentrated to give crude. Chromatographed (30% ethylacetate/hexanes) to give product 107 (0.0075 g, 0.012 mmol, 88%.) ¹H NMR (CDCl₃) δ 9.02 (s, 1H), 8.15 (d, 1H), 8.04 (s, 1H), 7.75 (d, 4H), 7.50 (dd, 1H), 7.27 (m, 6H), 7.08 (dd, 2H), 5.18 (s, 1H), 4.80 (s, 2H), 4.21 (s, 2H), 3.31 (q, 2H), 1.59 (m, 2H), 1.41 (m, 2H), 0.99 (t, 3H.) MS: 590 (M+1)

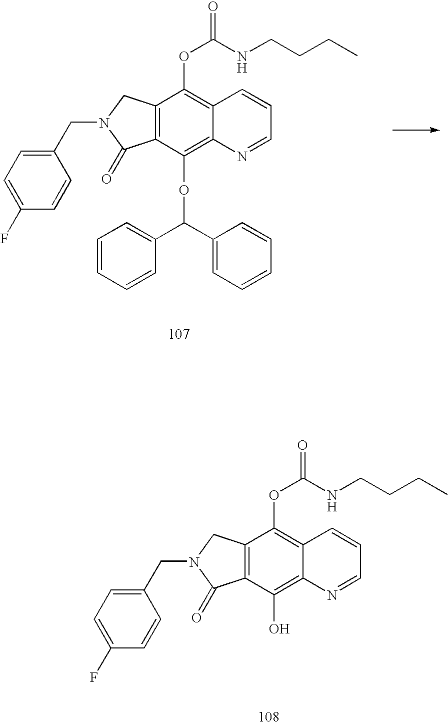

### Example 108

Carbamate 107 (0.007 g, 0.012 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 108 (0.0028 g, 0.0066 mmol, 56%.) ¹H NMR (CDCl₃) δ 8.98 (s, 1H), 8.27 (d, 1H), 7.59 (dd, 1H), 7.31 (dd, 2H), 7.06 (dd, 2H), 5.19 (s, 1H), 4.77 (s, 2H), 4.37 (s, 2H), 3.32 (q, 2H), 1.65 (m, 2H), 1.44 (m, 2H), 1.01 (t, 3H) MS: 424 (M+1), 422 (M−1)

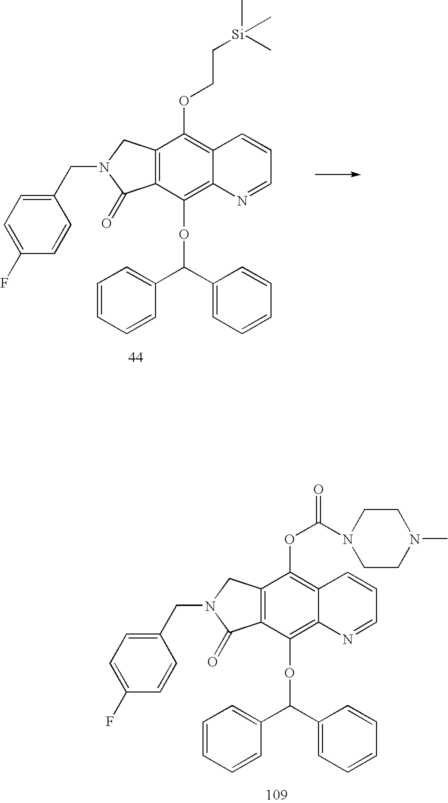

### Example 109

Trimethylsilylethyl ether 44 (0.01 g, 0.0169 mmol) was dissolved in 0.5 mL dry tetrahydrofuran. To this was added triethylamine (0.014 mL, 0.0339 mmol) and 1 M tetrabutylammonium fluoride solution in tetrahydrofuran (0.0339 mL, 0.0339 mmol.) Stirred at room temperature 10 minutes until starting material consumed. Diluted with dichloromethane, washed with washed with 1M HCl solution, saturated NaHCO₃, saturated brine, concentrated to give crude. Dissolved in 0.5 mL dichloromethane, added catalytic dimethylaminopyridine, triethylamine (0.042 mL, 0.1017 mmol) and cooled to 0° C. To this was added a 1M solution of triphosgene in dichloromethane (0.1017 mL, 0.1017 mmol) and stirred 30 minutes. Methyl piperazine (0.0168 mL, 0.1521 mmol) was then added and stirred at room temperature for fifteen minutes. Diluted with dichloromethane, washed with brine, concentrated volatiles to give crude. Chromatographed (50% ethylacetate/hexanes to 10% methanol/ethylacetate) to give product 109 (0.0055 g, 0.009 mmol, 53%.) ¹H NMR (CDCl₃) δ 9.04 (d, 1H), 8.10 (d, 1H), 8.03 (s, 1H), 7.75 (d, 4H), 7.52 (dd, 1H), 7.27 (m, 8H), 7.05 (dd, 2H), 4.80 (s, 2H), 4.22 (s, 2H), 3.77 (br s, 2H), 3.58 (br s, 2H), 2.48 (br s, 4H), 2.37 (s, 3H.) MS: 617 (M+1)

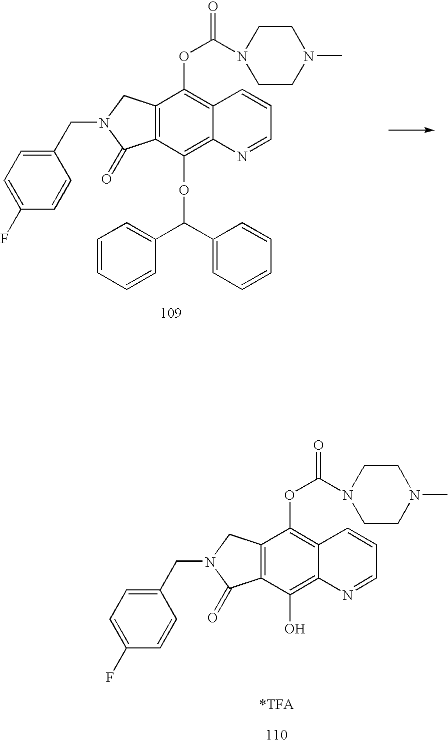

### Example 110

Carbamate 109 (0.007 g, 0.01136 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 110 (0.004 g, 0.007 mmol, 63%.) ¹H NMR (CDCl₃) δ 9.00 (s, 1H), 8.11 (d, 1H), 7.59 (dd, 1H), 7.35 (dd, 2H), 7.08 (dd, 2H), 4.78 (s, 2H), 4.35 (s, 2H), 3.50 (br m, 8H), 2.93 (s, 3H.), ¹⁹F NMR: −76.2 MS: 451 (M+1), 449 (M−1)

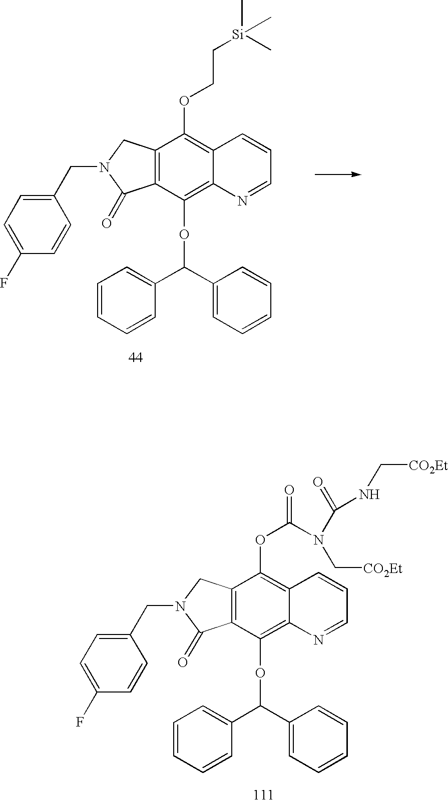

### Example 111

Trimethylsilylethyl ether 44 (0.02 g, 0.0339 mmol) was dissolved in 0.5 mL dry tetrahydrofuran. Triethylamine (0.0188 mL, 0.135 mmol) and 1 M tetrabutylammonium fluoride solution in tetrahydrofuran (0.0678 mL, 0.0678 mmol) were added. The mixture was stirred at room temperature for 10 minutes until starting material consumed. The mixture was diluted with dichloromethane, washed with 1M HCl solution, saturated NaHCO₃, saturated brine, and concentrated to give crude. The crude residue was dissolved in 0.5 mL dichloromethane, and catalytic dimethylaminopyridine, triethylamine (0.0188 mL, 0.135 mmol) and ethyl isocyanatoacetate (Aldrich, St. Louis, Mo., 0.011 mL, 0.1017 mmol) were added and stirred at room temperature (Satchell and Satchell, Chem. Soc. Rev. (1975) 4:231-250; R. G. Arnold et al., Chem. Soc. (1957) 57:47-76). After four hours, starting material was consumed. The mixture was diluted with dichloromethane, washed with 1M HCl, brine, and concentrated in vacuo to give crude product. The crude product was chromatographed on silica gel (10% to 50% ethylacetate/hexanes) to give product 111 (0.0118 g, 0.156 mmol, 46%) ¹H NMR (CDCl₃) δ 9.07 (d, 1H), 8.73 (s, 1H), 8.17 (d, 1H), 8.08 (s, 1H), 7.76 (d, 4H), 7.57 (dd, 1H), 7.27 (m, 8H), 7.08 (dd, 2H), 4.81 (s, 2H), 4.74 (s, 2H), 4.20 (m, 4H), 4.07 (d, 4H), 1.27 (m, 6H). MS: 749 (M+1), 747 (M−1).

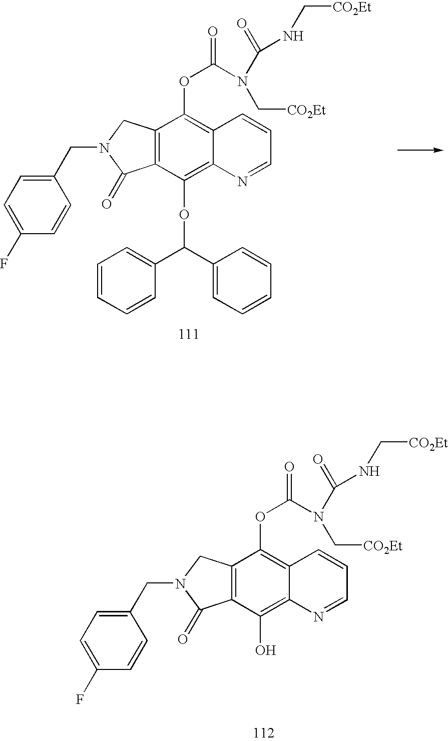

### Example 112

Carbamate 111 (0.011 g, 0.0177 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 112 (0.0056 g, 0.0095 mmol, 54%.) ¹H NMR (CDCl₃) δ 8.99 (s, 1H), 8.76 (s, 1H), 8.27 (d, 1H), 7.63 (dd, 1H), 7.35 (dd, 2H), 7.09 (dd, 2H), 4.79 (d, 4H), 4.33 (d, 2H), 4.23 (m, 4H), 4.09 (d, 2H), 1.30 (m, 6H.) MS: 583 (M+1), 581 (M−1)

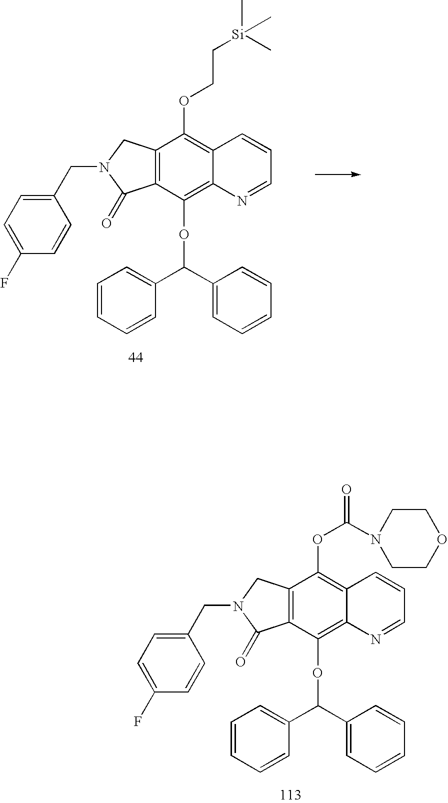

### Example 113

Trimethylsilylethyl ether 44 (0.02 g, 0.0339 mmol) was dissolved in 0.5 mL dry tetrahydrofuran. To this was added triethylamine (0.019 mL, 0.14 mmol) and 1 M tetrabutylammonium fluoride solution in tetrahydrofuran (0.0678 mL, 0.0678 mmol.) Stirred at room temperature 10 minutes until starting material consumed. Diluted with dichloromethane, washed with washed with 1M HCl solution, saturated NaHCO₃, saturated brine, concentrated to give crude. Dissolved in 0.5 mL dichloromethane, added catalytic dimethylaminopyridine, triethylamine (0.019 mL, 0.14 mmol) and cooled to 0° C. To this was added a 1M solution of triphosgene in dichloromethane (0.0678 mL, 0.0678 mmol) and stirred 60 minutes. Morpholine (0.009 mL, 0.1016 mmol) was then added and stirred at room temperature for 30 minutes. Diluted with dichloromethane, washed with 1M HCl, brine, concentrated volatiles to give crude. Chromatographed (40% ethylacetate/hexanes to 60% ethylacetate/hexanes) to give product 113 (0.0176 g, 0.028 mmol, 86%.) ¹H NMR (CDCl₃) δ 9.05 (d, 1H), 8.09 (d, 1H), 8.04 (s, 1H), 7.75 (d, 4H), 7.53 (dd, 1H), 7.27 (m, 8H), 7.06 (dd, 2H), 4.81 (s, 1H), 4.23 (s, 2H), 3.78 (br s, 6H), 3.56 (br s, 2H.) MS: 604 (M+1), 602 (M−1)

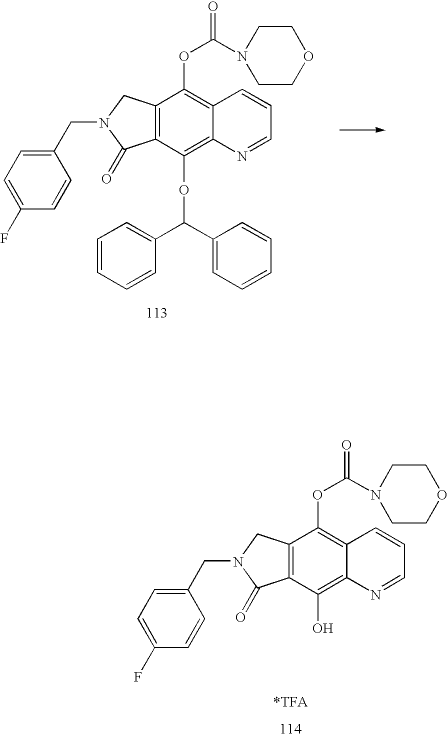

### Example 114

Carbamate 113 (0.017 g, 0.028 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 114 (0.0085 g, 0.015 mmol, 55%.) ¹H NMR (CDCl₃) δ 9.02 (s, 1H), 8.24 (d, 1H), 7.62 (dd, 1H), 7.33 (dd, 2H), 7.07 (dd, 2H), 4.78 (s, 2H), 4.39 (s, 2H), 3.82 (br s, 6H), 3.60 (br s, 2H.) ¹⁹F NMR: −76.2 MS: 438 (M+1), 436 (M−1)

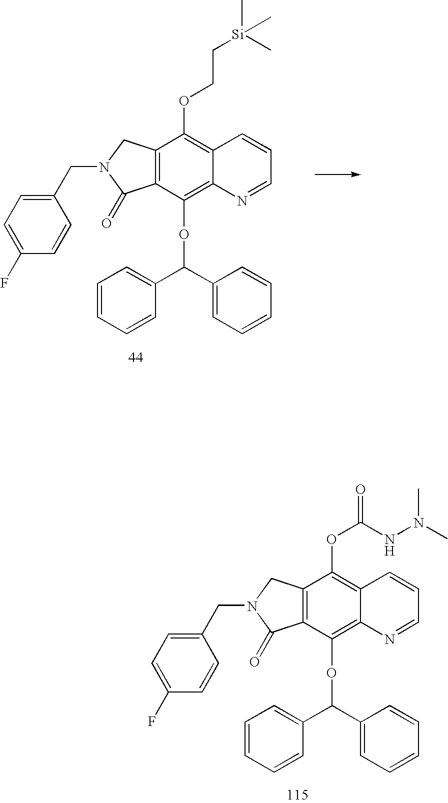

### Example 115

Trimethylsilylethyl ether 44 (0.02 g, 0.0339 mmol) was dissolved in 0.5 mL dry tetrahydrofuran. To this was added diisopropylethylamine (0.024 mL, 0.135 mmol) and 1 M tetrabutylammonium fluoride solution in tetrahydrofuran (0.0678 mL, 0.0678 mmol.) Stirred at room temperature 10 minutes until starting material consumed. Diluted with dichloromethane, washed with washed with 1M HCl solution, saturated NaHCO₃, saturated brine, concentrated to give crude. Dissolved in 0.5 mL dichloromethane, added catalytic dimethylaminopyridine, diisopropylethylamine (0.024 mL, 0.135 mmol) and cooled to 0° C. To this was added a 1M solution of triphosgene (bis[trichloromethyl] carbonate) in dichloromethane (0.0678 mL, 0.0678 mmol) and stirred 45 minutes. Dimethylhydrazine (0.01 mL, 0.135 mmol) was then added and stirred at room temperature for 20 minutes. Diluted with dichloromethane, washed with saturated NH₄Cl solution, concentrated volatiles to give crude. Chromatographed (10% ethylacetate/hexanes to 60% ethylacetate/hexanes) and purified by preparatory TLC plate (60% ethylacetate/hexanes) to give product 115 (0.004 g, 0.0069 mmol, 20%.) ¹H NMR (CDCl₃) δ 9.05 (d, 1H), 8.11 (d, 1H), 8.04 (s, 1H), 7.75 (d, 4H), 7.5 (dd, 1H), 7.27 (m, 8H), 7.07 (dd, 2H), 6.14 (s, 1H), 4.80 (s, 2H), 4.23 (s, 2H), 2.70 (6H.) MS: 577 (M+1)

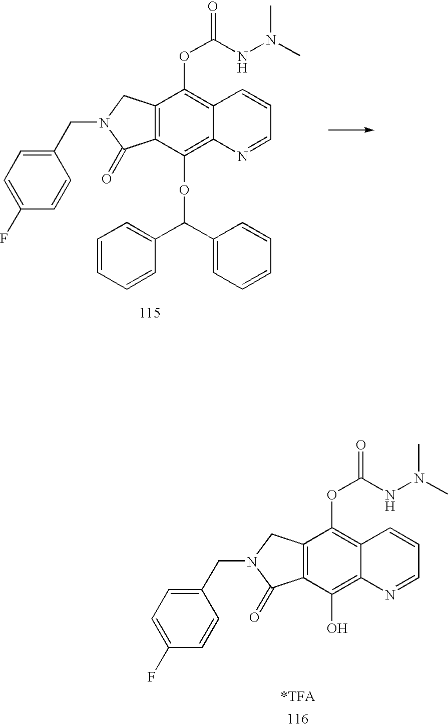

### Example 116

Carbamate 115 (0.009 g, 0.0156 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 116 (0.003 g, 0.0057 mmol, 37%.) ¹H NMR (CDCl₃) δ 8.96 (s, 1H), 8.24 (d, 1H), 7.56 (dd, 1H), 7.33 (dd, 2H), 7.06 (dd, 2H), 4.76 (s, 2H), 4.39 (s, 2H), 2.74 (s, 3H.) ¹⁹F NMR: −76.1 MS: 411 (M+1), 409 (M−1)

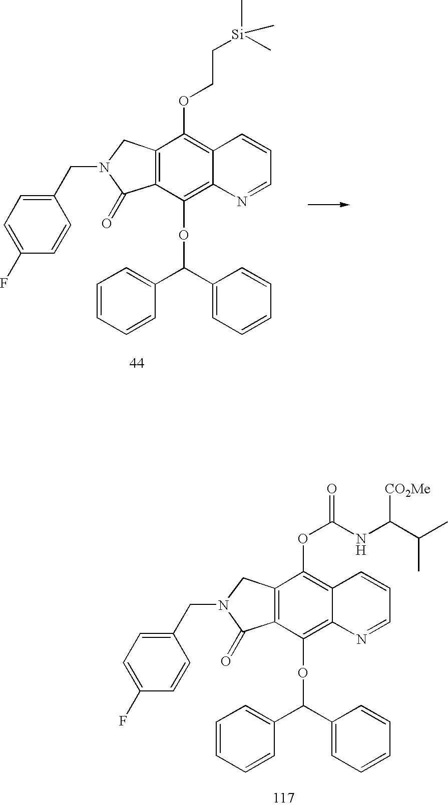

### Example 117

Trimethylsilylethyl ether 44 (0.02 g, 0.0339 mmol) was dissolved in 0.5 mL dry tetrahydrofuran. To this was added triethylamine (0.0188 mL, 0.135 mmol) and 1 M tetrabutylammonium fluoride solution in tetrahydrofuran (0.0678 mL, 0.0678 mmol.) Stirred at room temperature 10 minutes until starting material consumed. Diluted with dichloromethane, washed with washed with 1M HCl solution, saturated, saturated brine, concentrated to give crude. Dissolved in 0.5 mL dichloromethane, added catalytic dimethylaminopyridine, triethylamine (0.0188 mL, 0.135 mmol) and methyl (s)-(−)-2-isocyanato-3-methyl butyrate (0.0048 mL, 0.0339 mmol) and stirred at room temperature. After 4.5 hours, starting material consumed. Diluted with dichloromethane, washed with saturated NH₄Cl solution, concentrated organics to give crude. Chromatographed (10% to 50% ethylacetate/hexanes) to give product 117 (0.0085 g, 0.013 mmol, 39%.) ¹H NMR (CDCl₃) δ 9.03 (s, 1H), 8.17 (d, 1H), 8.05 (s, 1H), 7.75 (4H), 7.52 (dd, 1H), 7.27 (m, 8H), 7.07 (dd, 2H), 5.70 (d, 1H), 4.80 (s, 2H), 4.21 (s, 2H), 3.79 (s, 3H), 2.28 (dsp, 1H), 1.03 (d, 3H), 0.98 (d, 3H.) MS: 649 (M+1)

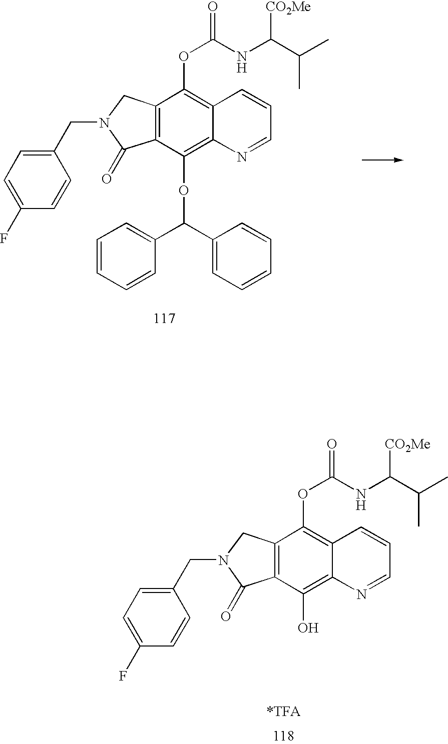

### Example 118

Carbamate 117 (0.004 g, 0.006 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 118 (0.0027 g, 0.0046 mmol, 76%.) ¹H NMR (CDCl₃) δ 9.00 (s, 1H), 8.31 (d, 1H), 7.60 (dd, 1H), 7.33 (dd, 2H), 7.09 (dd, 2H), 5.76 (d, 1H), 4.77 (s, 2H), 4.36 (s, 2H), 3.81 (s, 3H), 2.28 (dsp, 1H), 1.06 (d, 3H), 1.00 (d, 3H.) ¹⁹F NMR: −76.2 MS: 482 (M+1), 480 (M−1)

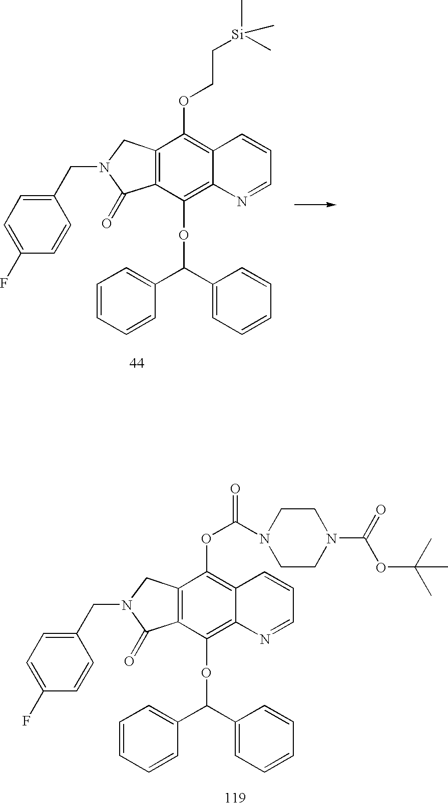

### Example 119

Trimethylsilylethyl ether 44 (0.2 g, 0.339 mmol) was dissolved in 3 mL dry tetrahydrofuran. To this was added triethylamine (0.139 mL, 1 mmol) and 1 M tetrabutylammonium fluoride solution in tetrahydrofuran (0.0678 mL, 0.0678 mmol.) Stirred at room temperature 10 minutes until starting material consumed. Diluted with dichloromethane, washed with washed with 1M HCl solution, saturated brine, concentrated to give crude. Dissolved in 3 mL dichloromethane, added catalytic dimethylaminopyridine, triethylamine (0.754 mL, 5.4 mmol) and cooled to 0° C. To this was added a 1M solution of triphosgene in dichloromethane (0.1356 mL, 0.1356 mmol) and stirred 50 minutes. BOC-piperazine (0.37 g, 2 mmol) was then added and stirred at room temperature for 30 minutes. Diluted with dichloromethane, washed with 1M HCl, brine, concentrated volatiles to give crude. Chromatographed (10% to 30% acetone/toluene) to give product 119 (0.1158 g, 0.166 mmol, 49%.) ¹H NMR (CDCl₃) δ 9.04 (d, 1H), 8.09 (d, 1H), 8.04 (s, 1H), 7.75 (d, 4H), 7.50 (dd, 1H), 7.27 (m, 8H), 7.05 (dd, 2H), 4.80 (s, 2H), 4.22 (s, 2H), 3.73 (br s, 2H), 3.53 (br s, 4H), 1.51 (s, 9H.) MS: 688 (M+1)

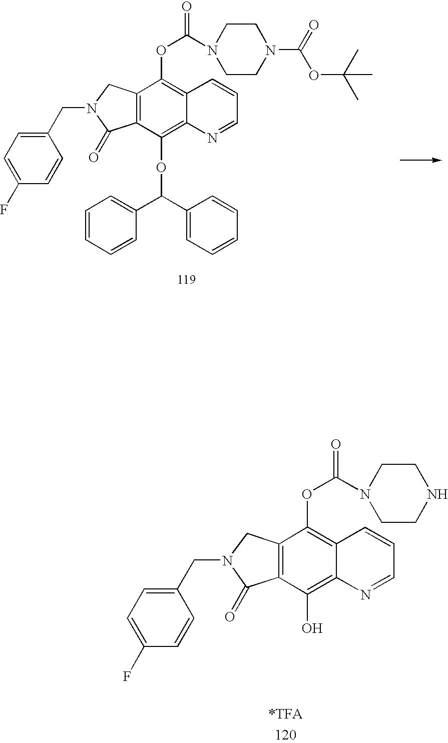

### Example 120

Carbamate 119 (0.057 g, 0.082 mmol) was dissolved in 1 mL of dichloromethane. To this was added 0.4 mL of triethylsilane and 0.2 mL of trifluoroacetic acid. Stirred at room temperature and after ten minutes complete by TLC. Concentrated off volatiles, azeotroped with toluene to give crude. Then dissolved in 1 mL dichloromethane, 1 ml trifluoroacetic acid. Stirred at room temperature for one hour. Concentrated off volatiles, azeotroped with toluene to give crude. Triturated twice with 1:1 diethylether/hexanes to give product 120 (0.0317 g, 0.059 mmol, 72%.) ¹H NMR (CD₃SOCD₃) δ 8.97 (br m, 2H), 8.40 (d, 1H), 7.75 (dd, 1H), 7.35 (dd, 2H), 7.23 (dd, 2H), 4.71 (s, 2H), 4.38 (s, 2H), 3.91 (br s, 2H), 3.24 (br s, 4H.) ¹⁹F NMR: −74.5 MS: 437 (M+1), 435 (M−1)

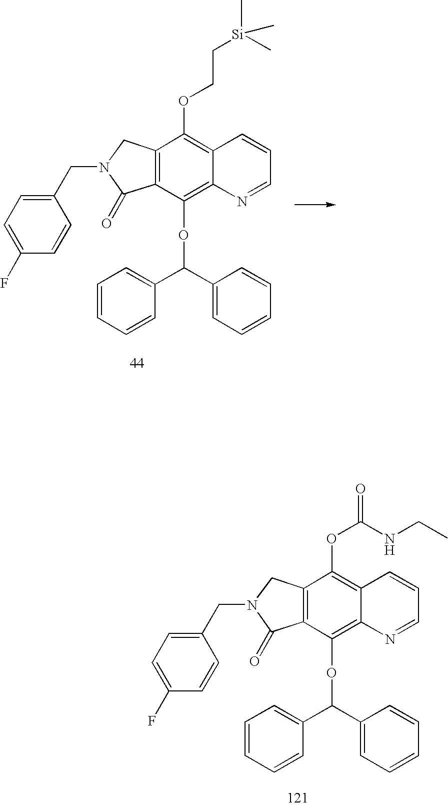

### Example 121

Trimethylsilylethyl ether 44 (0.035 g, 0.0596 mmol) was dissolved in 0.8 mL dry tetrahydrofuran. To this was added triethylamine (0.05 mL, 0.358 mmol) and 1 M tetrabutylammonium fluoride solution in tetrahydrofuran (0.119 mL, 0.119 mmol.) Stirred at room temperature 10 minutes until starting material consumed. Diluted with dichloromethane, washed with washed with 1M HCl solution, saturated brine, concentrated to give crude. Dissolved in 0.8 mL dichloromethane, added triethylamine (0.05 mL, 0.358 mmol) and ethyl isocyanate (0.0046 mL, 0.0595 mmol) and stirred at room temperature. After 6 hours, starting material consumed. Diluted with dichloromethane, washed with saturated brine, concentrated organics to give crude. Chromatographed (10% to 50% ethylacetate/hexanes) to give product 121 (0.0112 g, 0.023 mmol, 39%.) ¹H NMR (CDCl₃) δ 9.05 (s, 1H), 8.17 (d, 1H), 8.04 (s, 1H), 7.76 (d, 4H), 7.50 (dd, 1H), 7.27 (m, 8H), 7.05 (dd, 2H), 4.80 (s, 2H), 4.23 (s, 2H), 3.33 (q, 2H), 1.27 (t, 3H.) MS: 562 (M+1)

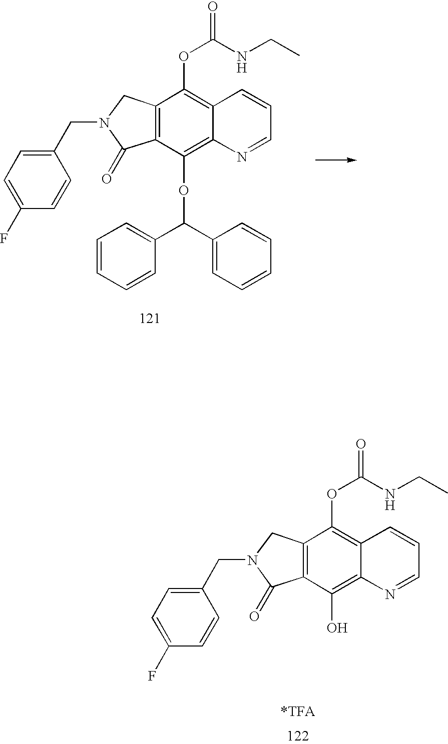

### Example 122

Carbamate 121 (0.0112 g, 0.023 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 122 (0.0033 g, 0.0076 mmol, 33%.) ¹H NMR (CDCl₃) δ 9.06 (s, 1H), 8.37 (d, 1H), 7.64 (dd, 1H), 7.33 (dd, 2H), 7.06 (dd, 2H), 5.24 (s, 1H), 4.77 (s, 2H), 4.39 (s, 2H), 3.38 (q, 2H), 1.30 (t, 3H.) ¹⁹F NMR: −76.2 MS: 397 (M+1), 395 (M−1)

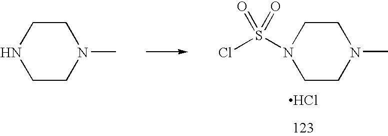

### Example 123

N-Methyl piperazine (0.33 mL, 3 mmol) was added slowly and with caution to a mixture of sulfuryl chloride (0.72 mL, 9 mmol) in 6 mL of acetonitrile. The solution was heated to reflux for 15 hours. After starting material consumed, solution concentrated to oil, azeotroped with toluene (2×), concentrated to give crude product which was triturated with diethylether to give the product 123 as a pale brown solid (0.5 g, 71%.) ¹H NMR (CD₃SOCD₃) δ 3.90 (br s, 2H), 3.59 (br s, 2H.), 3.38 (br. S, 4H), 2.67 (s, 3H); MS: 200 (M+1).

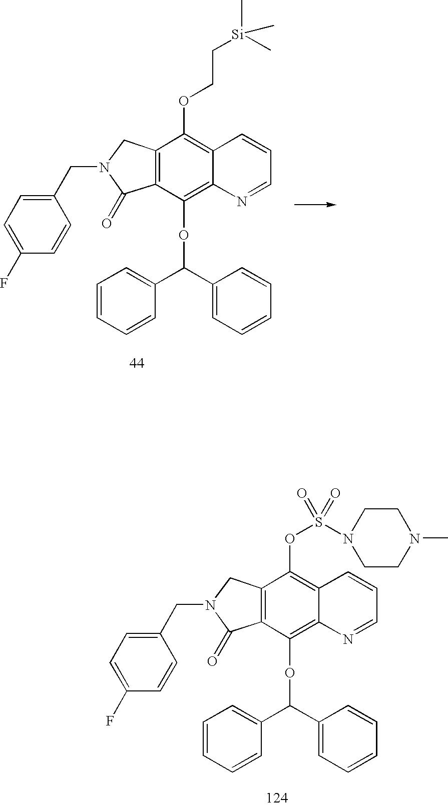

### Example 124

Trimethylsilylethyl ether 44 (0.03 g, 0.0508 mmol) was dissolved in 0.5 mL dry tetrahydrofuran. Triethylamine (0.021 mL, 0.1525 mmol) and 1 M tetrabutylammonium fluoride solution in tetrahydrofuran (0.1016 mL, 0.1016 mmol.) were added. The mixture was stirred at room temperature 10 minutes until starting material was consumed, then diluted with dichloromethane, washed with washed with 1M HCl solution, saturated brine, and concentrated. The crude product was dissolved in 0.5 mL dichloromethane. Catalytic dimethylaminopyridine, triethylamine (0.035 mL, 0.254 mmol) and methyl piperazine sulfamoyl chloride HCl salt 123 (0.024 g, 0.1016 mmol) were added and stirred at room temperature. After 15 hours, starting material was consumed. The mixture was diluted with dichloromethane, washed with saturated brine, and concentrated organics to give crude product which was chromatographed (1% to 10% methanol/dichloromethane) to give product 124 (0.016 g, 0.0246 mmol, 48%.) ¹H NMR (CDCl₃) δ 9.07 (s, 1H), 8.38 (d, 1H), 8.08 (s, 1H), 7.75 (d, 4H), 7.55 (dd, 1H), 7.27 (m, 8H), 7.08 (dd, 2H), 4.81 (s, 2H), 4.46 (s, 2H), 3.51 (br s, 4H), 2.54 (br s, 4H), 3.35 (s, 3H.) MS: 653 (M+1)

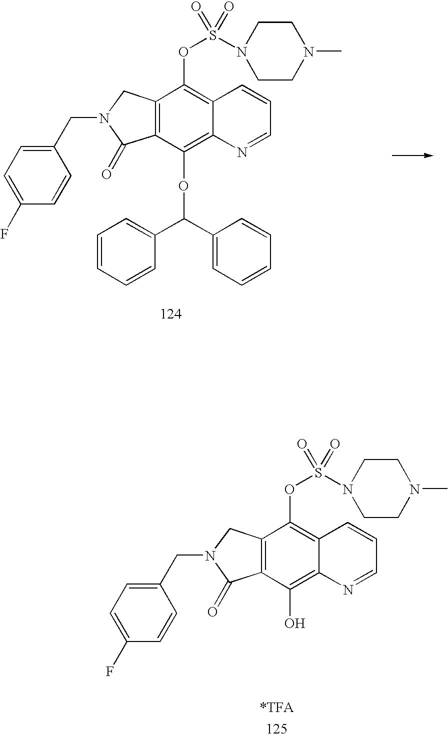

### Example 125

Sulfamate 124 (0.016 g, 0.0246 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 125 (0.008 g, 0.0133 mmol, 54%.) ¹H NMR (CDCl₃) δ 9.02 (s, 1H), 8.37 (d, 1H), 7.67 (dd, 1H), 7.33 (dd, 2H), 7.06 (dd, 2H), 4.80 (s, 2H), 4.57 (s, 2H), 3.95 (br s, 4H), 3.29 (br s, 4H), 2.89 (s, 3H.) ¹⁹F NMR: −76.2 MS: 487 (M+1), 485 (M−1)

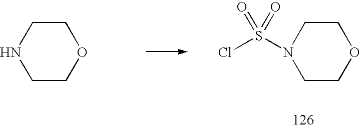

### Example 126

Morpholine (0.436 mL, 5 mmol) was added slowly and with caution to a mixture of sulfuryl chloride (1.205 mL, 15 mmol) in 5 mL acetonitrile. Heated to reflux and stirred for 24 hours. After starting material consumed, solution concentrated to oil, azeotroped with toluene (2×), concentrated to give crude product 126 stored as a 2M solution in dichloromethane (0.999 g, 5 mmol, 100%.) ¹H NMR (CD₃SOCD₃) δ 3.80 (br s, 4H), 3.28 (br s, 4H.) MS: 186 (M+1)

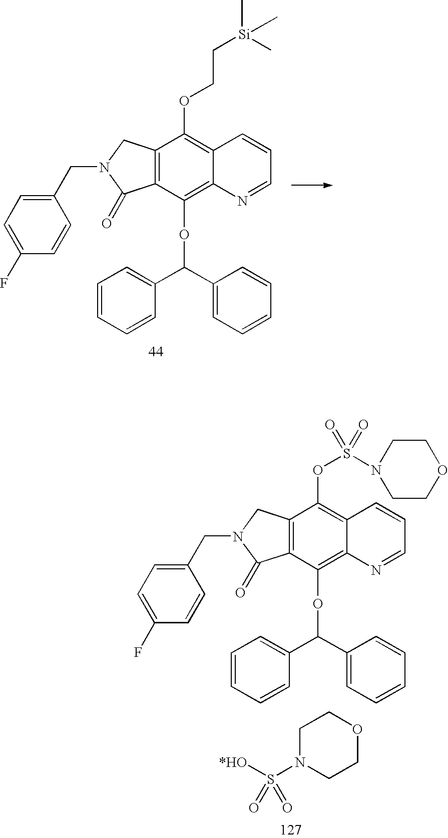

### Example 127

Trimethylsilylethyl ether 44 (0.027 g, 0.0457 mmol) was dissolved in 0.5 mL dry tetrahydrofuran. To this was added triethylamine (0.025 mL, 0.1828 mmol) and 1 M tetrabutylammonium fluoride solution in tetrahydrofuran (0.0915 mL, 0.0915 mmol.) Stirred at room temperature 10 minutes until starting material consumed. Diluted with dichloromethane, washed with washed with 1M HCl solution, saturated brine, concentrated to give crude. Dissolved in 0.5 mL dichloromethane, added catalytic dimethylaminopyridine, triethylamine (0.025 mL, 0.1828 mmol) and 2 M morpholine sulfamoyl chloride solution 126 in dichloromethane (0.05 g, 0.10 mmol) and stirred at room temperature. After 1.5 hours, starting material consumed. Diluted with dichloromethane, washed with saturated brine, concentrated organics to give crude. Chromatographed (10% to 40% ethylacetate/hexanes) to give product 127 (0.0199 g, 0.031 mmol, 68%.) ¹H NMR (CDCl₃) δ 9.07 (s, 1H), 8.35 (d, 1H), 8.09 (s, 1H), 7.75 (d, 4H), 7.56 (dd, 1H), 7.27 (m, 8H), 7.05 (dd, 2H), 4.82 (s, 2H), 4.46 (s, 2H), 3.81 (m, 4H), 3.75 (m, 4H), 3.48 (m, 4H), 3.27 (m, 4H.) MS: 790 (M+1)

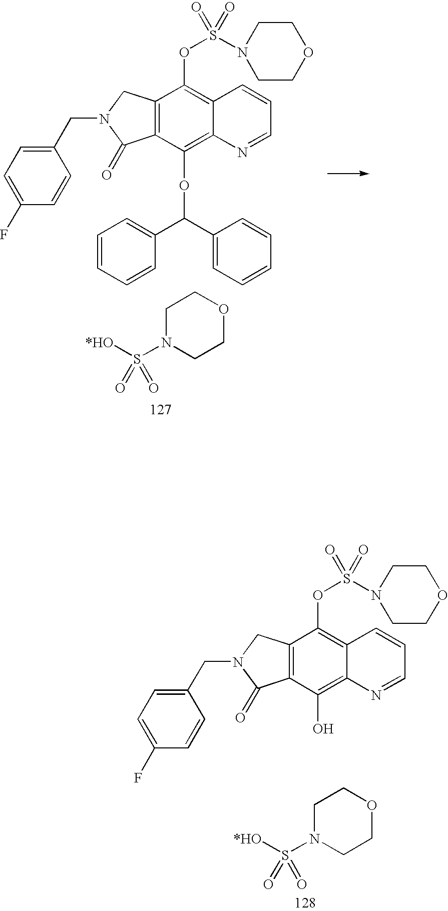

### Example 128

Sulfamate 127 (0.095 g, 0.012 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethylether/hexanes to give product 128 (0.0054 g, 0.0086 mmol, 71%.) ¹H NMR (CDCl₃) δ 9.00 (s, 1H), 8.45 (d, 1H), 7.65 (dd, 1H), 7.33 (dd, 2H), 7.10 (dd, 2H), 4.79 (s, 2H), 4.59 (s, 2H), 3.86 (m, 4H), 3.76 (m, 4H), 3.59 (m, 4H), 3.28 (m, 4H.) MS: 624 (M+1), 622 (M−1)

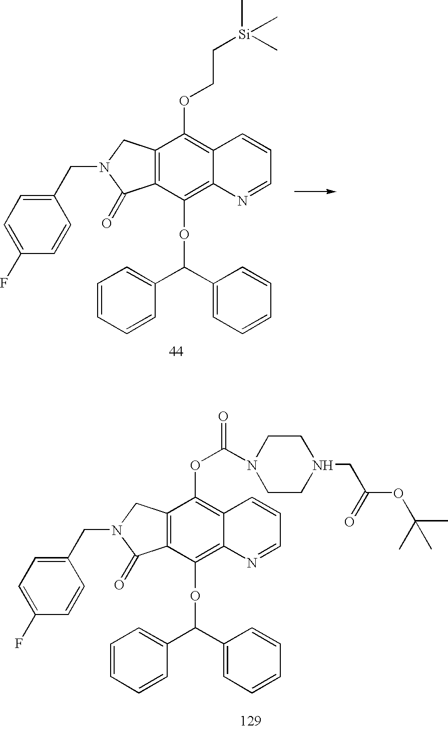

### Example 129

Trimethylsilylethyl ether 44 (0.1 g, 0.169 mmol) was dissolved in 2 mL dry tetrahydrofuran. To this was added triethylamine (0.094 mL, 0.676 mmol) and 1 M tetrabutylammonium fluoride solution in tetrahydrofuran (0.339 mL, 0.339 mmol.) Stirred at room temperature 10 minutes until starting material consumed. Diluted with dichloromethane, washed with washed with 1M HCl solution, saturated brine, concentrated to give crude. Dissolved in 1.5 mL dichloromethane, added catalytic dimethylaminopyridine, triethylamine (0.139 mL, 1 mmol) and cooled to 0° C. To this was added triphosgene (0.1 g, 0.339 mmol) and stirred 40 minutes. BOC-aminopiperidine (0.135 g, 0.678 mmol) was then added and stirred at room temperature for 10 minutes. Diluted with dichloromethane, washed with 1M HCl, brine, concentrated volatiles to give crude. Chromatographed (10% to 50% ethylacetate/hexanes) to give product 129 (0.072 g, 0.097 mmol, 59%.) ¹H NMR (CDCl₃) δ 9.04 (dd, 1H), 8.07 (d, 1H), 8.04 (s, 1H), 7.74 (d, 4H), 7.50 (dd, 1H), 7.27 (m, 8H), 7.06 (dd, 2H), 4.80 (s, 2H), 4.48 (br s, 1H), 4.28 (m, 1H), 4.21 (s, 3H), 3.71 (br s, 2H), 3.21 (dd, 2H), 3.03 (dd, 2H), 1.48 (s, 9H.) MS: 717 M+1)

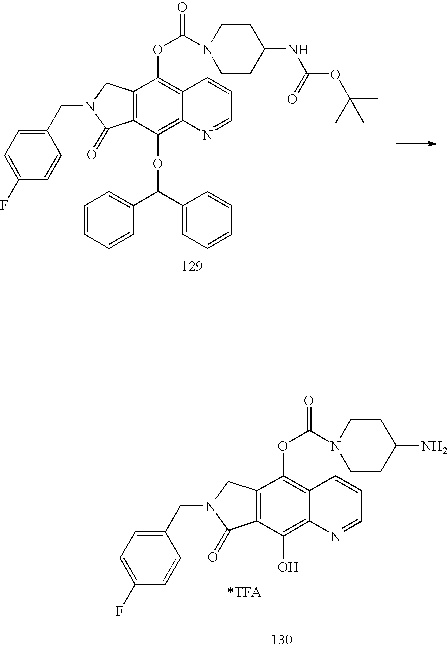

### Example 130

Carbamate 129 (0.07 g, 0.097 mmol) was dissolved in 2 mL of dichloromethane. To this was added 0.5 mL of triethylsilane and 0.2 mL of trifluoroacetic acid. Stirred at room temperature and after ten minutes complete by TLC. Concentrated off volatiles, azeotroped with toluene to give crude. Then dissolved in 1.5 mL dichloromethane, 1.5 ml trifluoroacetic acid. Stirred at room temperature for one hour. Concentrated off volatiles, azeotroped with toluene to give crude. Triturated twice with 1:1 diethylether/hexanes to give product 130 (0.0329 g, 0.058 mmol, 60%.) ¹H NMR (CD₃SOCD₃) δ 8.98 (s, 1H), 8.22 (d, 1H), 7.95 (s, 2H), 7.74 (dd, 1H), 7.35 (dd, 2H), 7.19 (dd, 2H), 4.70 (s, 2H), 4.35 (s, 3H), 4.00 (br s, 1H), 3.44 (br s, 7H.) ¹⁹F NMR: −74.1 MS: 451 (M+1), 449 (M−1)

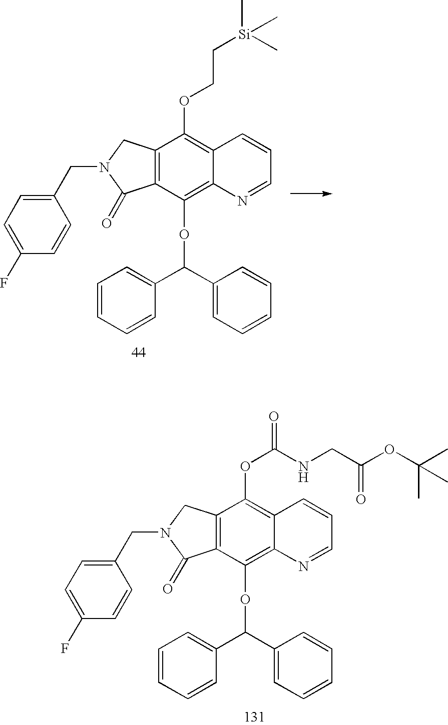

### Example 131

Triphosgene (0.06 g, 0.2032 mmol) was added to 0.5 mL dichloromethane and cooled to 0° C. To this was slowly added glycine tertiary-butyl ester HCl salt (0.034 g, 0.2032 mmol) and triethylamine (0.14 mL, 1 mmol) and stirred at 0° C. Stirred thirty minutes until starting material consumed. Simultaneously, in a separate flask trimethylsilylethyl ether compound 44 was dissolved in 0.5 mL tetrahydrofuran. To this was added triethylamine (0.028 mL, 0.2032 mmol) and 1M tetrabutylammonium fluoride in tetrahydrofuran (0.1016 mL, 0.1016 mmol) and stirred at room temperature. After 20 minutes, diluted with dichloromethane, washed with 1M HCl solution and brine, concentrated to give crude. At 0° C., crude dissolved in 0.5 mL dichloromethane and added to the glycine isocyanate prepared in situ above. Stirred at 0° C. for 5 minutes, then stirred for one hour at room temperature. Diluted with dichloromethane, washed with 1M HCl solution, brine, concentrated to give crude. Chromatographed (10% to 40% ethylacetate/hexanes) to give product 131 (0.017 g, 0.026 mmol, 52%.) ¹H NMR (CDCl₃) δ 9.03 (d, 1H), 8.20 (dd, 1H), 8.05 (s, 1H), 7.75 (d, 4H), 7.51 (dd, 1H), 7.27 (m, 8H), 7.04 (dd, 2H), 5.66 (s, 1H), 4.79 (s, 2H), 4.23 (s, 2H), 3.93 (d, 2H), 1.5 (s, 9H.) MS: 648 (M+1)

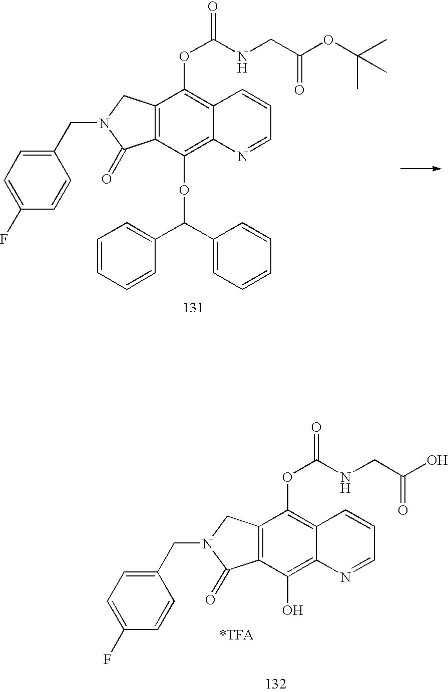

### Example 132

Carbamate 131 (0.017 g, 0.026 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Then dissolved in 0.5 mL dichloromethane, 0.2 mL triethylsilane, 0.2 ml trifluoroacetic acid. Stirred at room temperature for three hours. Concentrated off volatiles, azeotroped with toluene to give crude. Triturated with 1:1 diethylether/hexanes to give product 132 (0.0088 gm, 0.021 mmol, 80%.) ¹H NMR (CD₃SOCD₃) δ 8.97 (s, 1H), 8.40 (s, 1H), 8.30 (d, 1H), 7.74 (dd, 1H), 7.37 (m, 2H), 7.23 (m, 2H), 4.69 (s, 2H0, 4.32 (s, 2H), 3.76 (d, 2H.) ¹⁹F NMR: −74.3 MS: 426 (M+1), 424 (M−1)

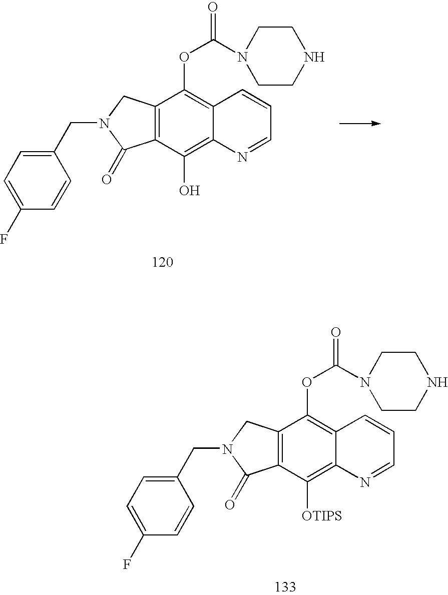

### Example 133

Carbamate 120 (0.019 g, 0.0435 mmol) was dissolved in 0.5 mL of dichloroethane. To this was added triethylamine (0.072 mL, 0.52 mmol) and triisopropylsilyl chloride (0.058 mL, 0.26 mmol) and stirred at 50° C. After 19 hours, starting material consumed, diluted with dichloromethane, washed with 1M HCl solution, brine and concentrated to give crude. Chromatographed to give product 133 (0.012 g, 0.0203 mmol, 47%.) ¹H NMR (CDCl₃) δ 8.86 (s, 1H), 8.06 (d, 1H), 7.54 (dd, 1H), 7.33 (dd, 2H), 7.08 (dd, 2H), 4.78 (s, 2H), 4.21 (s, 4H), 4.01 (br s, 2H), 3.35 (br s, 4H), 11.58 (m, 1H), 1.16 (d, 18H.) MS: 593 (M+1)

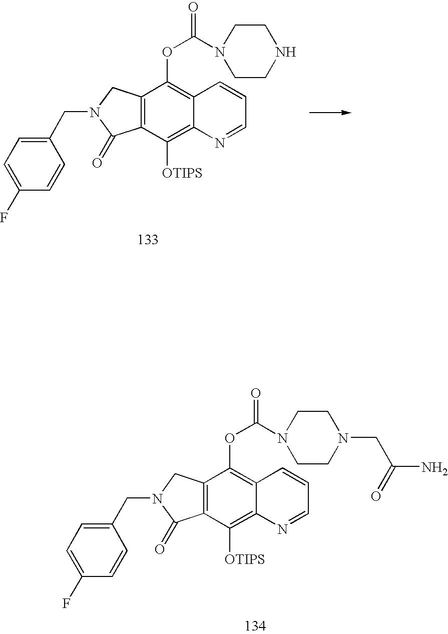

### Example 134

Piperazine carbamate 133 (0.012 g, 0.0203 mmol) was dissolved 0.5 mL of acetonitrile and 0.2 mL dichloromethane. To this was added Cs₂CO₃ (0.0325 g, 0.1 mmol) and 2-bromoacetamide (0.009 g, 0.0608 mmol.) Stirred at room temperature for 3.5 days, until starting material was consumed. Diluted with dichloromethane, washed with saturated NH₄Cl solution, concentrated to give product 134 (0.0037 g, 0.0057, 28%.) ¹H NMR (CDCl₃) δ 8.87 (dd, 1H), 8.11 (d, 1H), 7.73 (s, 1H), 7.53 (dd, 1H), 7.34 (dd, 2H), 7.07 (dd, 2H), 4.78 (s, 2H), 4.23 (s, 2H), 3.84 (br s, 2H), 3.64 (br s, 2H), 3.14 (s, 2H), 2.62 (br s, 4H), 1.58 (m, 3H), 1.17 (d, 18H.) MS: 650 (M+1)

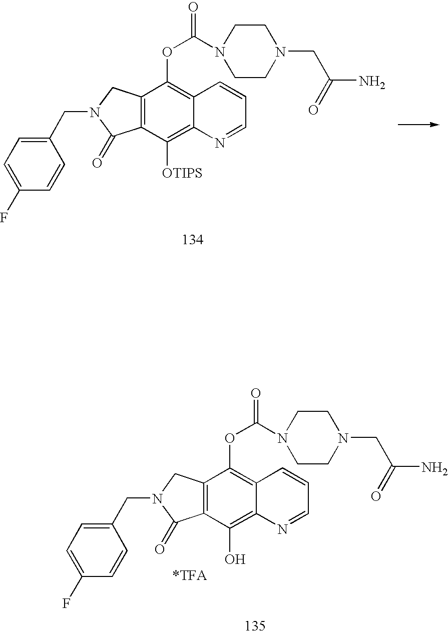

### Example 135

Mono-carbamate 134 (0.0037 g, 0.0057 mmol) was dissolved in 0.2 mL of dichloromethane. To this was added trifluoroacetic acid (0.009 mL, 0.114 mmol) and stirred at room temperature. After twenty hours, concentrated off volatiles, azeotroped with toluene (2×), concentrated to give crude. Triturate with 1:1 diethylether/hexanes to give product 135 (0.0015 g, 0.0024 mmol, 43%.) ¹H NMR (CD₃OD) δ 8.96 (s, 1H), 8.38 (s, 1H), 7.75 (m, 2H), 7.39 (dd, 2H), 7.11 (dd, 2H), 4.87 (s, 2H), 4.42 (s, 2H), 4.0 (br m, 8H), 3.3 (s, 2H.) ¹⁹F: −77.73 MS: 494 (M+1), 492 (M−1)

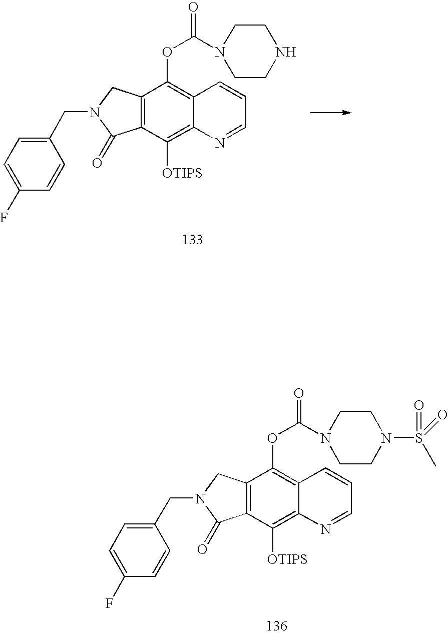

### Example 136

Piperazine carbamate 133 (0.033 g, 0.056 mmol) was dissolved in 0.5 mL dichloromethane. To this was added catalytic dimethylaminopyridine, triethylamine (0.031 mL, 0.225 mmol) and methanesulfonyl chloride (0.0087 mL, 0.112 mmol) at 0° C. After five minutes, continued stirring at room temperature. After one hour starting material consumed. Diluted with dichloromethane, washed with saturated NH₄Cl solution, dried (Na₂SO₄) concentrated to give crude. Chromatographed (10% to 60% ethylacetate/hexanes) to give product 136 (0.013 g, 0.019 mmol, 35%.) ¹H NMR (CDCl₃) δ 8.88 (dd, 1H), 8.08 (d, 1H), 7.53 (dd, 1H), 7.33 (dd, 2H), 7.05 (dd, 2H), 4.79 (s, 2H), 4.23 (s, 2H), 3.93 (br s, 2H), 3.72 (br s, 2H), 3.37 (br s, 4H), 2.88 (s, 3H), 1.58 (m, 3H), 1.17 (d, 18H.) MS: 671 (M+1)

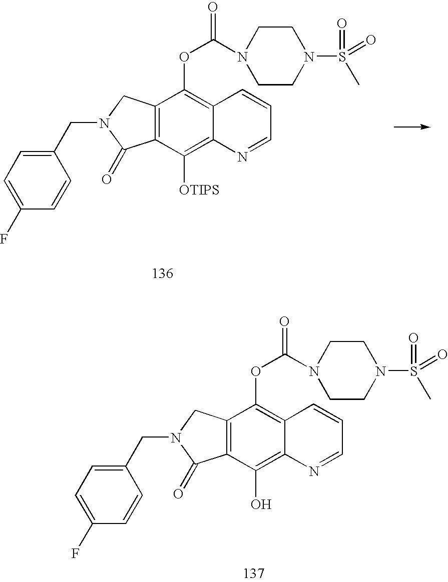

### Example 137

Mono-carbamate 136 (0.013 g, 0.019 mmol) was dissolved in 0.5 mL of dichloromethane. To this was added trifluoroacetic acid (0.056 mL, 0.72 mmol) and stirred at room temperature. After twenty hours, concentrated off volatiles, azeotroped with toluene (2×), concentrated to give crude. Triturate with 1:1 diethylether/hexanes to give product 137 (0.0066 g, 0.013 mmol, 68%.) ¹H NMR (CDCl₃) δ 9.01 (s, 1H), 8.17 (s, 1H), 7.61 (s, 1H), 7.27 (dd, 2H), 7.07 (dd, 2H), 4.79 (s, 2H), 4.37 (s, 2H), 3.93 (br s, 2H), 3.72 (br s, 2H), 3.39 (br s, 4H), 2.89 (s, 3H.) MS: 515 (M+1), 513 (M−1)

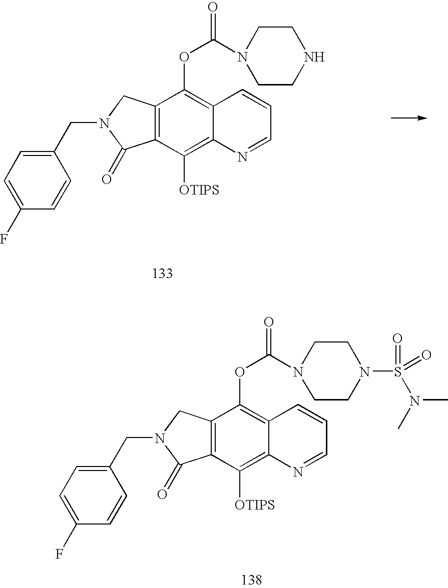

### Example 138

Piperazine carbamate 133 (0.033 g, 0.056 mmol) was dissolved in 0.5 mL dichloromethane. To this was added catalytic dimethylaminopyridine, triethylamine (0.031 mL, 0.225 mmol) and methanesulfonyl chloride (0.0087 mL, 0.112 mmol) at 0° C. After five minutes, continued stirring at room temperature. After one hour starting material consumed. Diluted with dichloromethane, washed with saturated NH₄Cl solution, dried (Na₂SO₄) concentrated to give crude. Chromatographed (10% to 50% ethylacetate/hexanes) to give product 138 (0.012 g, 0.017 mmol, 31%.) ¹H NMR (CDCl₃) δ 8.87 (dd, 1H), 8.09 (d, 1H), 7.53 (dd, 1H), 7.31 (dd, 2H), 7.07 (dd, 2H), 4.79 (s, 2H), 4.23 (s, 2H), 3.87 (br s, 2H), 3.66 (br s, 2H), 3.35 (br s, 4H), 2.89 (s, 6H), 1.56 (m, 3H), 1.17 (d, 18H.) MS: 700 (M+1)

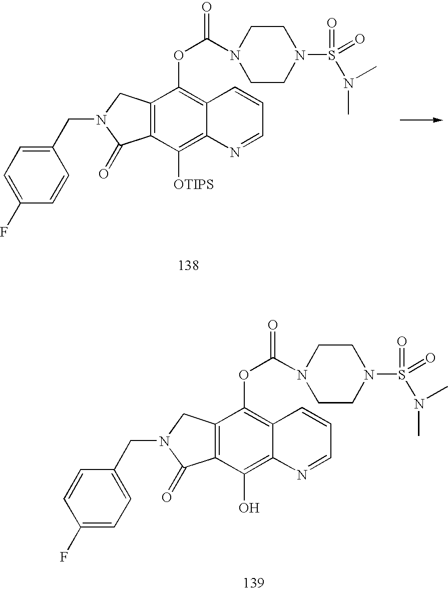

### Example 139

Mono-carbamate 138 (0.012 g, 0.017 mmol) was dissolved in 0.5 mL of dichloromethane. To this was added trifluoroacetic acid (0.056 mL, 0.72 mmol) and stirred at room temperature. After twenty hours, concentrated off volatiles, azeotroped with toluene (2×), concentrated to give crude. Triturate with 1:1 diethylether/hexanes to give product 139 (0.0039 g, 0.007 mmol, 42%.) ¹H NMR (CDCl₃) δ 9.00 (s, 1H), 8.18 (d, 1H), 7.60 (s, 1H), 7.27 (dd, 2H), 7.07 (dd, 2H), 4.78 (s, 2H), 4.36 (s, 2H), 3.88 (br s, 2H), 3.67 (br s, 2H), 3.35 (br s, 4H), 2.89 (s, 6H.) MS: 544 (M+1), 542 (M−1)

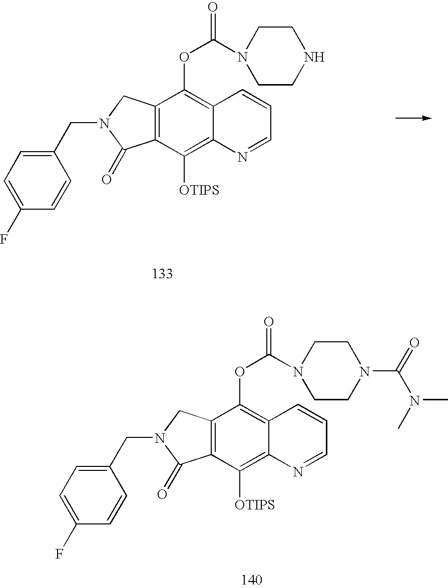

### Example 140

Piperazine carbamate 133 (0.033 g, 0.056 mmol) was dissolved in 0.5 mL dichloromethane. To this was added catalytic dimethylaminopyridine, triethylamine (0.031 mL, 0.225 mmol) and methanesulfonyl chloride (0.0087 mL, 0.112 mmol) at 0° C. After five minutes, continued stirring at room temperature. After one hour starting material consumed. Diluted with dichloromethane, washed with saturated NH₄Cl solution, dried (Na₂SO₄) concentrated to give crude. Chromatographed (50% to 100% ethylacetate/hexanes) to give product 140 (0.012 g, 0.018 mmol, 32%.) ¹H NMR (CDCl₃) δ 8.85 (dd, 1H), 8.11 (d, 1H), 7.52 (dd, 1H), 7.31 (dd, 2H), 7.07 (dd, 2H), 4.79 (s, 2H), 4.23 (s, 2H), 3.82 (br s, 2H), 3.60 (br s, 2H), 3.34 (br s, 4H), 2.91 (s, 6H), 1.56 (m, 3H), 1.17 (d, 18H.) MS: 664 (M+1)

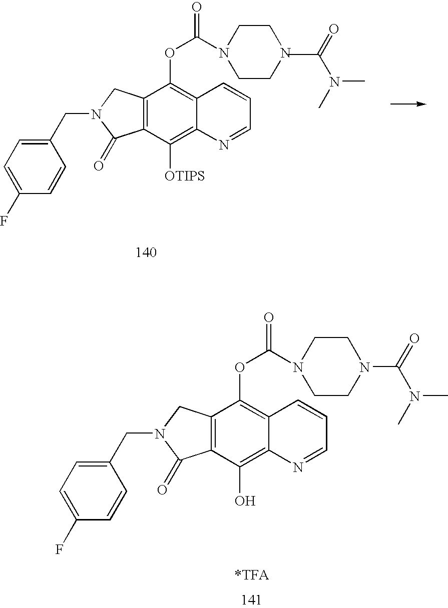

### Example 141

Mono-carbamate 140 (0.012 gm, 0.018 mmol) was dissolved in 0.5 mL of dichloromethane. To this was added trifluoroacetic acid (0.056 mL, 0.72 mmol) and stirred at room temperature. After twenty hours, concentrated off volatiles, azeotroped with toluene (2×), concentrated to give crude. Triturate with 1:1 diethylether/hexanes to give product 141 (0.0051 g, 0.0083 mmol, 46%.) ¹H NMR (CDCl₃) δ 9.00 (s, 1H), 8.21 (s, 1H), 7.60 (s, 1H), 7.27 (dd, 2H), 7.07 (dd, 2H), 4.76 (s, 2H), 4.37 (s, 2H), 3.83 (br s, 2H), 3.61 (br s, 2H), 3.35 (br s, 4H), 2.92 (s, 6H.) ¹⁹F: −76.3 MS: 508 (M+1), 506 (M−1)

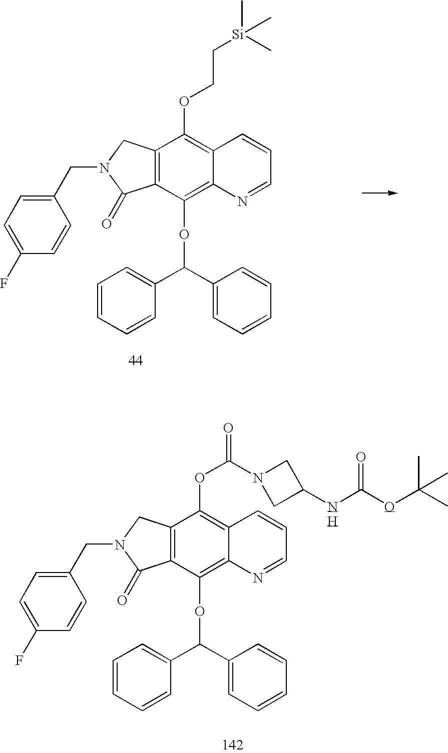

### Example 142

Trimethylsilylethyl ether 44 (0.03 g, 0.0508 mmol) was dissolved in 0.5 mL dry tetrahydrofuran. To this was added triethylamine (0.028 mL, 0.2032 mmol) and 1 M tetrabutylammonium fluoride solution in tetrahydrofuran (0.1016 mL, 0.1016 mmol.) Stirred at room temperature 10 minutes until starting material consumed. Diluted with dichloromethane, washed with washed with 1M HCl solution, saturated brine, dried (Na₂SO₄,) concentrated to give crude. Dissolved in 0.5 mL dichloromethane, added catalytic dimethylaminopyridine, triethylamine (0.08 mL, 0.6 mmol) and cooled to 0° C. To this was added triphosgene (0.03 g, 0.1016 mmol) and stirred 30 minutes. Azetid-3-yl carbamic acid t-butyl ester (0.035 g, 0.2032 mmol) and triethylamine (0.08 mL, 0.6 mmol) was then added and stirred at room temperature for 50 minutes. Diluted with dichloromethane, washed with 1M HCl, brine, dried (Na₂SO₄,) concentrated volatiles to give crude. Chromatographed (10% to 50% ethylacetate/hexanes) to give product 142 (0.024 g, 0.035 mmol, 69%.) ¹H NMR (CDCl₃) δ 9.04 (dd, 1H), 8.17 (d, 1H), 8.03 (s, 1H), 7.74 (d, 4H), 7.51 (dd, 1H), 7.27 (m, 8H), 7.08 (dd, 2H), 5.00 (m, 1H), 4.80 (s, 2H), 4.52 (m, 2H), 4.23 (s, 2H), 3.91 (m, 2H), 1.48 (s, 9H.) MS: 689 (M+1)

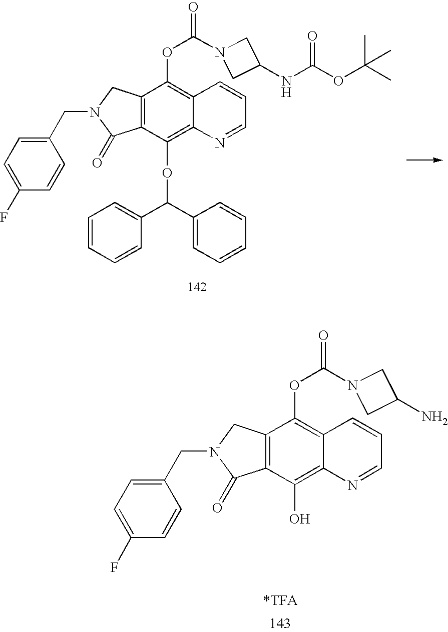

### Example 143

Carbamate 142 (0.024 g, 0.035 mmol) was dissolved in 0.5 mL of dichloromethane. To this was added 0.4 mL of triethylsilane and 0.2 mL of trifluoroacetic acid. Stirred at room temperature and after ten minutes complete by TLC. Concentrated off volatiles, azeotroped with toluene to give crude. Then dissolved in 0.75 mL dichloromethane, 0.75 ml trifluoroacetic acid. Stirred at room temperature for one hour. Concentrated off volatiles, azeotroped with toluene to give crude. Triturated twice with 1:1 diethylether/hexanes to give product 143 (0.0128 g, 0.024 mmol, 68%.) ¹H NMR (CD₃SOCD₃) δ 9.00 (s, 1H), 8.38 (br s, 3H), 7.75 (s, 1H), 7.36 (br s, 2H), 7.22 (br s, 2H), 4.72 (s, 2H), 4.32 (br m, 5H), 3.14 (br s, 2H.) ¹⁹F NMR: −74.0 MS: 423 (M+1), 421 (M−1)

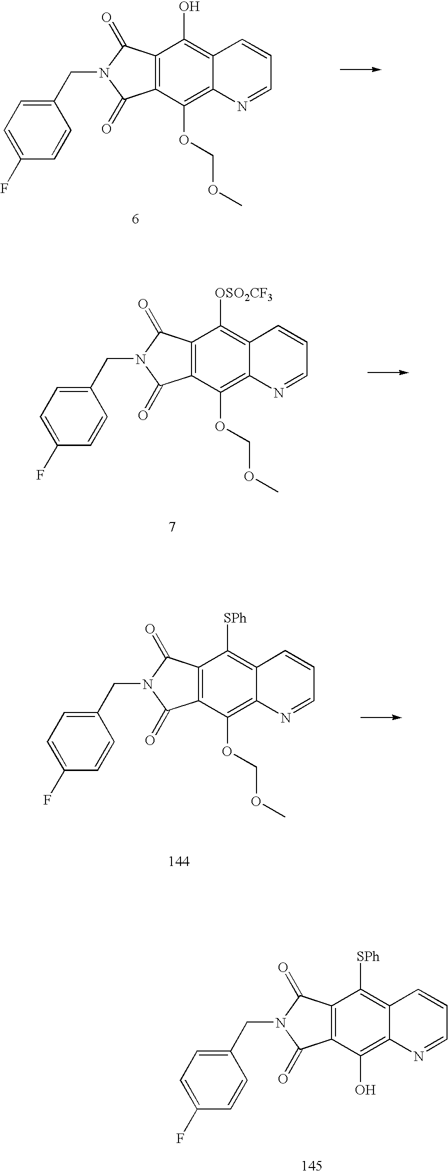

### Example 144

To crude triflate 7 (0.025 g, 0.048 mmol) in 1 mL of dichloroethane was added triethylamine (0.014 mL, 0.096 mmol) and benzenethiol (0.008 ml, 0.072 mmol) and the solution stirred at room temperature. After 15 hrs, the mixture was concentrated and chromatographed on silica gel eluting with EtOAc/hexanes to give compound 144 (0.01 g, 44%) as a yellow oil. ¹H NMR (CDCl₃) δ 9.2 (m, 2H), 7.6 (dd, 1H), 7.06 (m, 5H), 7.0 (t, 2H), 5.97 (s, 2H), 4.85 (s, 2H), 3.72 (s, 3H); MS: 474 (M+1)

### Example 145

MOM ether 144 (0.009 g, 0.019 mmol) in 1 mL of dichloromethane was treated with TFA (0.015 mL, 0.19 mmol) at room temperature for 15 hrs. The volatiles were removed in vacuo and the residue was triturated with diethylether to afford 7-(4-fluoro-benzyl)-9-hydroxy-5-phenylsulfanyl-pyrrolo[3,4-g]quinoline-6,8-dione 145 as a yellow solid. ¹H NMR (CDCl₃) δ 9.31 (d, 2H), 7.81 (m, 1H), 7.46 (dd, 2H), 7.17 (m, 5H), 7.04 (t, 2H), 5.97 (s, 2H), 4.88 (s, 2H); MS: 430 (M+1).

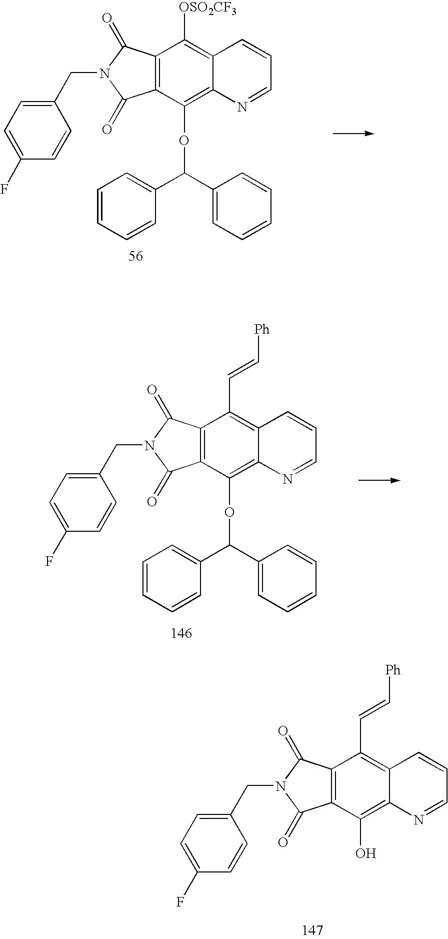

### Example 146

To the triflate 5 (0.045 g, 0.07 mmol) in toluene (0.7 mL)/ethanol (0.3 mL)/water (0.2 mL) were added potassium carbonate (0.037 g, 0.175 mmol), trans-phenylvinylbronic acid (0.016 g, 0.105 mmol) and tetrakis (triphenylphosphine)-palladium (0) (0.012 g, 0.011 mmol). The mixture in the flask was flushed with argon three times. It was heated to 120° C. under argon for 3 hours. Cooling to room temperature, it was diluted with EtOAc and washed with 1N HCl, saturated NaHCO3 and brine. The organic phase was dried (MgSO₄) and concentrated. The residue was chromatographed on a silica gel column, eluting with EtOAc/Hexane to afford the product 146 (0.031 g, 75%). MS: 613 (M+Na).

### Example 147

Compound 146 (8 mg, 0.013 mmol) was dissolved in dichloromethane (1 mL) at room temperature under nitrogen. Triethylsilane (0.034 mL) was added followed by TFA (0.02 mL) slowly. The mixture was stirred at room temperature for 30 min. The solvent was removed at reduced pressure. The crude product was triturated in diethylether/hexane to afford a yellow solid 7-(4-fluoro-benzyl)-9-hydroxy-5-styryl-pyrrolo[3,4-g]quinoline-6,8-dione 147 (0.005 g, 88%. ¹H NMR (CDCl₃): δ 8.99 (d, 1H), 8.88 (d, 1H), 8.05 (d, 1H), 7.67 (m, 3H), 7.36-7.52 (m, 5H), 7.01 (m, 3H), 4.87 (s, 2H); MS: 425 (M+1).

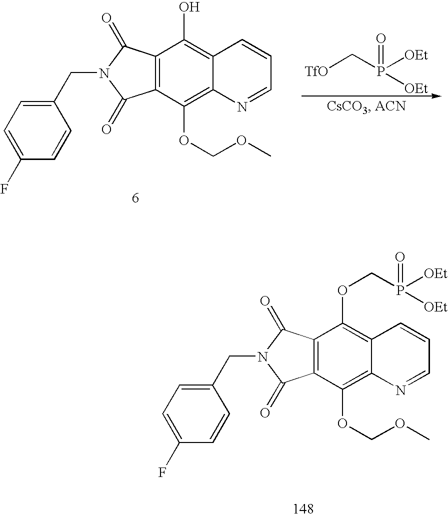

### Example 148

To a solution of trifluoromethanesulfonic acid diethoxyphosphorylmethyl ester (D. P. Phillion, et al, Tetra. Lett., 27 (1986) 1477-1480, 0.040 g, 0.104 mmol) dissolved in acetonitrile (0.75 mL) was added the phenol 6 (0.044 g, 0.146 mmol) and CsCO₃ (0.102 g, 0.314 mmol). The reaction mixture was stirred at room temperature for 3 hours under an inert atmosphere then filtered and concentrated in vacuo. The residue was purified by silica gel chromatography (3/1—ethylacetate/hexane) to afford the product 148 (0.014 g, 25%) as a solid: ¹H NMR (CDCl₃) δ 9.1 (d, 1H), 8.9 (d, 1H), 7.6 (dd, 1H), 7.5 (dd, 2H), 7.0 (t, 2H), 5.8 (s, 2H), 5.0 (d, 2H), 4.8 (s, 2H), 4.2 (m, 4H), 3.7 (s, 3H), 1.3 (t, 6H); ³¹P NMR (CDCl₃) δ 19.0; MS: 533 (M+1).

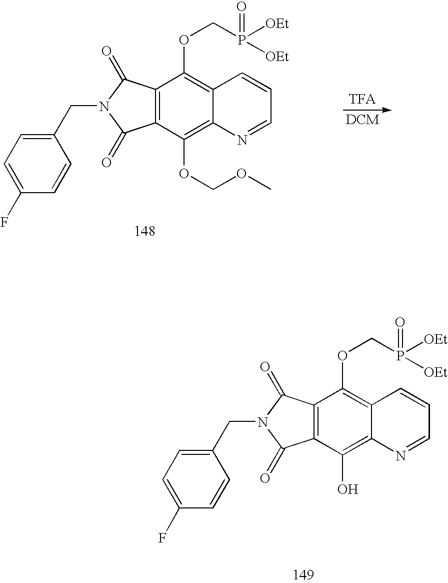

### Example 149

A solution of the phosphonate 148 (0.014 g, 0.026 mmol) in dichloromethane (0.96 mL) was treated with trifluoroacetic acid (0.020 mL, 0.260 mmol). The reaction mixture was stirred at room temperature under an inert atmosphere for 3 hours. The volatiles were removed in vacuo with toluene. The solid was triturated in diethylether/hexane to afford the product 149 (0.011 g, 86%) as a TFA salt: ¹H NMR (CDCl₃) δ 9.0 (d, 1H), 8.9 (d, 1H), 7.7 (dd, 1H), 7.5 (dd, 2H), 7.0 (t, 2H), 5.0 (d, 2H), 4.9 (s, 2H), 4.2 (m, 4H), 1.3 (s, 6H); ³¹P NMR (CDCl₃) δ 19.2; MS: 489 (M+1), 487 (M−1).

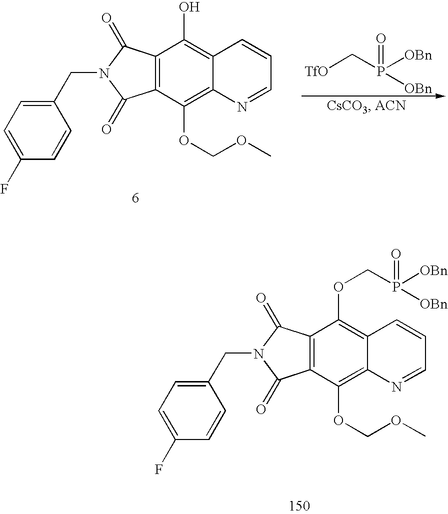

### Example 150

Dibenzyl hydroxymethyl phosphonate triflate was prepared from dibenzyl hydroxymethyl phosphonate (M. Krecmerova, et al, Czech. Chem. Commun., 55, 1990, 2521-2536) by the method of: Y. Xu, et al, J. Org. Chem., 61 (1996) 7697-7701. To a solution of dibenzyl hydroxymethyl phosphonate triflate (, 0.050 g, 0.131 mmol) dissolved in acetonitrile (1.87 mL) was added the phenol 6 (0.078 g, 0.183 mmol) and CsCO₃ (0.102 g, 0.314 mmol). The reaction mixture was stirred at room temperature for 3 hours under an inert atmosphere then filtered and concentrated in vacuo. The residue was purified by silica gel chromatography (1/1—ethylacetate/hexane) to afford the product 150 (0.030 g, 35%) as a solid: ¹H NMR (CDCl₃) δ 9.0 (d, 1H), 8.65 (d, 1H), 7.5 (dd, 2H), 7.4 (dd, 1H), 7.3 (m, 10H), 7.0 (t, 2H), 5.8 (s, 2H), 5.1 (m, 4H), 4.9 (d, 2H), 4.8 (s, 2H), 3.7 (s, 3H); ³¹P NMR (CDCl₃) δ 20.1; MS: 657 (M+1).

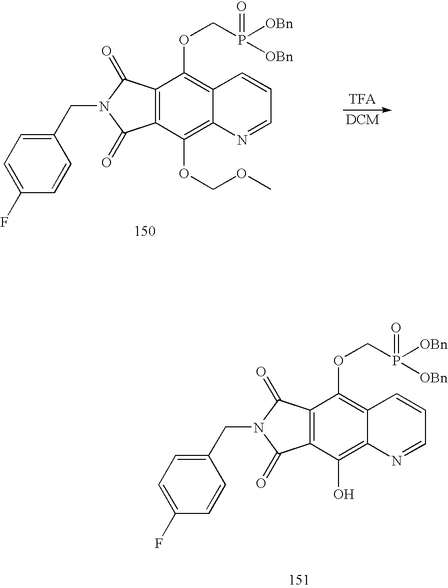

### Example 151

A solution of the phosphonate 150 (0.029 g, 0.044 mmol) in dichloromethane (1.6 mL) was treated with trifluoroacetic acid (0.034 mL, 0.44 mmol). The reaction mixture was stirred at room temperature under an inert atmosphere for 3 hours. The volatiles were removed in vacuo with toluene. The solid was triturated in diethylether/hexane to afford the product 151 (0.024 g, 89%) as a TFA salt: ¹H NMR (CDCl₃) δ 8.9 (d, 1H), 8.6 (d, 1H), 7.5 (dd, 2H), 7.45 (dd, 1H), 7.3-7.2 (m, 10H), 7.0 (t, 2H), 5.1-5.0 (m, 4H), 5.0 (d, 2H), 4.8 (s, 2H); ³¹P NMR (CDCl₃) δ 20.3; MS: 613 (M+1), 611 (M−1).

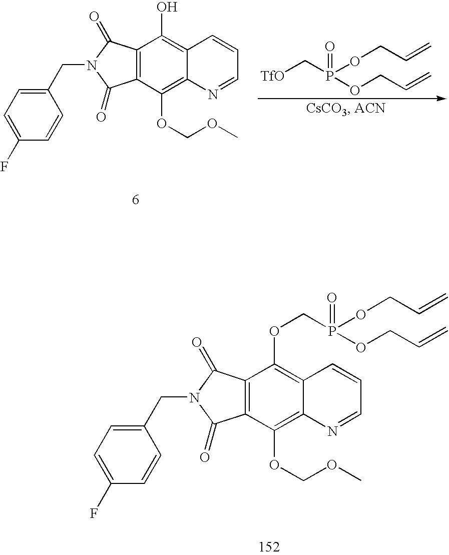

### Example 152

To a solution of diallyl hydroxymethyl phosphonate triflate (prepared by a method similar to: D. P. Phillion, et al, Tetra. Lett., 27 (1986) 1477-1480 and Y. Xu, et al, J. Org. Chem., 61 (1996) 7697-7701, 0.153 g, 0.471 mmol) dissolved in acetonitrile (6.7 mL) was added 7-(4-fluoro-benzyl)-5-hydroxy-9-methoxymethoxy-pyrrolo[3,4-g]quinoline-6,8-dione 6 (0.060 g, 0.157 mmol) and CsCO₃ (0.154 g, 0.471 mmol). The reaction mixture was stirred at room temperature for 2 hours under an inert atmosphere then filtered and concentrated in vacuo. The residue was purified by silica gel chromatography (3/1—ethylacetate/hexane) to afford [7-(4-fluoro-benzyl)-9-methoxymethoxy-6,8-dioxo-7,8-dihydro-6H-pyrrolo[3,4-g]quinolin-5-yloxymethyl]-phosphonic acid diallyl ester 152 (0.051 g, 59%) as a solid: ¹H NMR (CDCl₃) δ 9.05 (d, 1H), 8.85 (d, 1H), 7.6 (dd, 1H), 7.45 (dd, 2H), 7.0 (t, 2H), 5.9 (m, 2H), 5.8 (s, 2H), 5.3 (d, 2H), 5.2 (d, 2H), 5.0 (d, 2H), 4.8 (s, 2H), 4.6 (m, 4H), 3.7 (s, 3H); ³¹P NMR (CDCl₃) δ 19.9; MS: 557 (M+1).

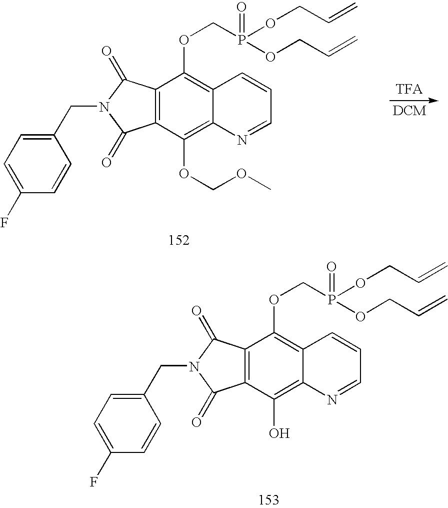

### Example 153

A solution of the phosphonate 152 (0.0065 g, 0.0117 mmol) in dichloromethane (0.425 mL) was treated with trifluoroacetic acid (0.009 mL, 0.117 mmol). The reaction mixture was stirred at room temperature under an inert atmosphere for 1 hour. The volatiles were removed in vacuo with toluene. The solid was triturated in diethylether/hexane to afford the product 153 (0.006 g, 100%) as a TFA salt: ¹H NMR (CDCl₃) δ 9.0 (d, 1H), 8.9 (d, 1H), 7.7 (dd, 2H), 7.5 (dd, 1H), 7.0 (t, 2H), 5.9 (m, 2H), 5.3 (d, 2H), 5.2 (d, 2H), 5.0 (d, 2H), 4.85 (s, 2H), 4.6 (m, 4H); ³¹P NMR (CDCl₃) δ 20.0; MS: 513 (M+1), 511 (M−1).

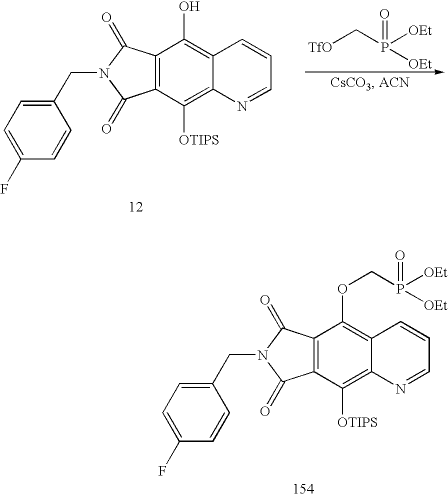

### Example 154

Diethyl hydroxymethyl phosphonate triflate was prepared from diethyl hydroxymethyl phosphonate (Aldrich, St. Louis, Mo.,) by the method of: D. P. Phillion, et al, Tetra. Lett., 27, 1986, 1477-1480. To a solution of diethyl hydroxymethyl phosphonate triflate (0.61 g, 0.202 mmol) dissolved in acetonitrile (2.9 mL) was added the phenol 12 (0.100 g, 0.202 mmol) and CsCO₃ (0.198 g, 0.607 mmol). The reaction mixture was stirred at room temperature for 3 hours under an inert atmosphere then filtered and concentrated in vacuo. The residue was purified by silica gel chromatography (1/1—ethylacetate/hexane) to afford the product 154 (0.130 g, 100%) as a solid: ¹H NMR (CDCl₃) δ 8.95 (d, 1H), 8.9 (d, 1H), 7.6 (dd, 1H), 7.5 (dd, 2H), 7.0 (t, 2H), 4.9 (d, 2H), 4.8 (s, 2H), 4.2 (m, 4H), 1.5 (m, 3H), 1.3 (t, 6H), 1.2 (d, 18H); ³¹P NMR (CDCl₃) δ 19.5; MS: 645 (M+1).

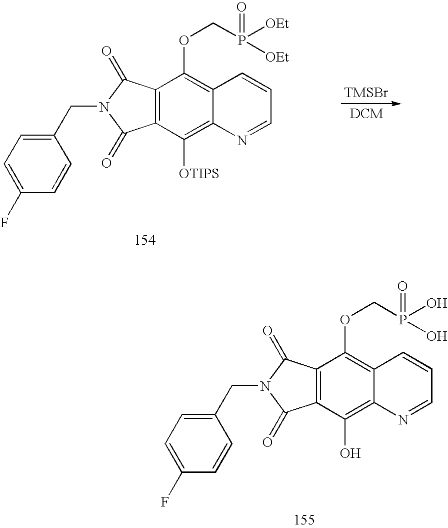

### Example 155

A solution of the phosphonate 154 (0.020 g, 0.031 mmol) in dichloromethane (0.311 mL) was treated with trimethylsilane bromide (0.0246 mL, 0.186 mmol). The reaction mixture was stirred at room temperature overnight under an inert atmosphere. The volatiles were removed in vacuo with methanol. The solid was washed with dichloromethane to afford the diacid 155 (0.010 g, 77%): ¹H NMR (CD₃OD) δ 9.5 (d, 1H), 9.2 (d, 1H), 8.2 (dd, 1H), 7.5 (dd, 2H), 7.1 (t, 2H), 5.0 (d, 2H), 4.9 (s, 2H); ³¹P NMR (CD₃OD) δ 16.2; MS: 433 (M+1), 431 (M−1).

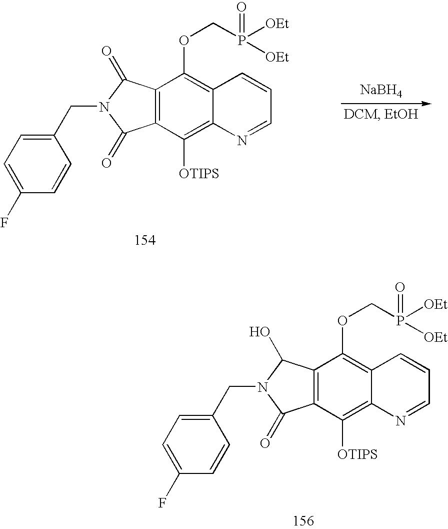

### Example 156

To a solution of the phosphonate 154 (0.038 g, 0.059 mmol) dissolved in dichloromethane (0.297 mL) and ethanol (0.297 mL) was added sodium borohydride (0.475 mL, 0.237 mmol). The reaction mixture stirred at room temperature overnight under an inert atmosphere and then was concentrated in vacuo. The residue was dissolved in ethylacetate and washed with saturated NH₄Cl and brine. The organic phase was dried (MgSO₄) then concentrated in vacuo. The residue was purified by silica gel chromatography (1/99—methanol/dichloromethane) to afford the product 156 (0.022 g, 59%): ¹H NMR (CDCl₃) δ 8.9 (d, 1H), 8.4 (d, 1H), 7.5 (dd, 1H), 7.4 (dd, 2H), 7.0 (t, 2H), 6.0 (s, 1H), 5.8 (bs, 1H), 5.2 (d, 1H), 4.6-4.4 (m, 2H), 4.4 (d, 1H), 4.3-4.2 (m, 4H), 1.6 (m, 3H), 1.4 (m, 6H), 1.15 (d, 18H); ³¹P NMR (CDCl₃) δ 20.95; MS: 647 (M+1).

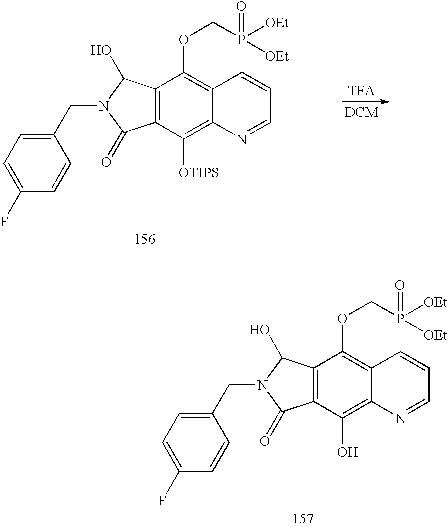

### Example 157

A solution of the phosphonate 156 (0.025 g, 0.039 mmol) in dichloromethane (1.41 mL) was treated with trifluoroacetic acid (0.030 mL, 0.390 mmol). The reaction mixture was stirred at room temperature under an inert atmosphere overnight. The volatiles were removed in vacuo, azeotroping to dryness with toluene. The solid was triturated in diethylether/hexane to afford the product 157 (0.021 g, 100%) as a TFA salt: ¹H NMR (CDCl₃) δ 9.0 (d, 1H), 8.6 (d, 1H), 7.6 (dd, 1H), 7.4 (dd, 2H), 7.0 (t, 2H), 6.2 (bs, 1H), 6.0 (s, 1H), 5.1 (d, 1H), 4.7-4.5 (m, 2H), 4.5 (d, 1H), 4.25 (m, 4H), 1.4 (m, 6H); ³¹P NMR (CDCl₃) δ 20.1; MS: 491 (M+1), 489 (M−1).

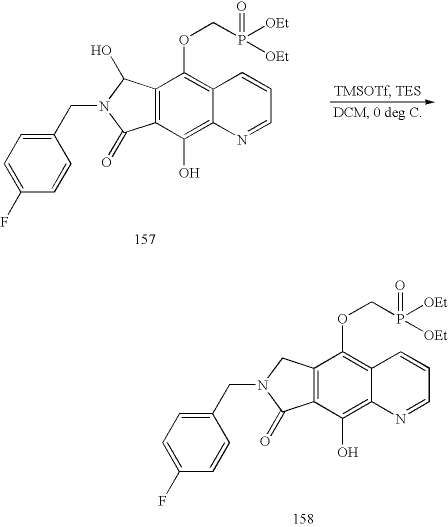

### Example 158

A solution of phosphonate 157 (0.0185 g, 0.0378 mmol) in dichloromethane (0.455 mL) was cooled to 0° C. Triethylsilane (0.0603 mL, 0.378 mmol) and then trimethylsilane triflate (0.0205 mL, 0.113 mmol) were added. The reaction stirred for 15 minutes under an inert atmosphere. The mixture was partitioned between dichloromethane and water. The organic phase was washed with saturated NaHCO₃ then dried (MgSO₄) and concentrated in vacuo. The solid was triturated in diethylether/hexane to afford the product 158 (0.015 g, 84%): ¹H NMR (CDCl₃) δ 9.0 (dd, 1H), 8.6 (dd, 1H), 7.6 (dd, 1H), 7.35 (dd, 2H), 7.15 (t, 2H), 4.8 (s, 2H), 4.5 (s, 2H), 4.3 (d, 2H), 4.2 (m, 4H), 1.3 (t, 6H); ³¹P NMR (CDCl₃) δ 18.7; MS: 475 (M+1).

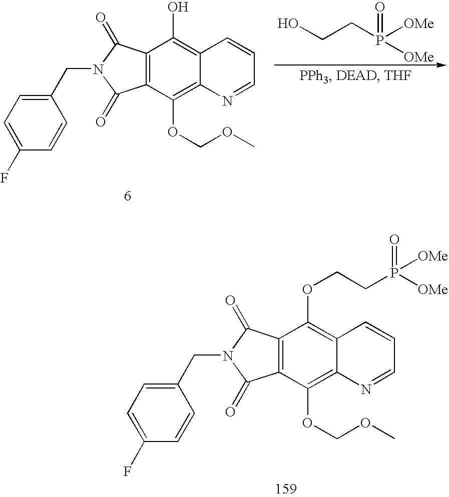

### Example 159

To a solution of phenol 6 (0.063 g, 0.165 mmol) dissolved in THF (0.86 mL) was added dimethyl hydroxyethyl phosphonate (0.076 g, 0.495 mmol), triphenylphosphine (0.108 g, 0.412 mmol), and diethyl azodicarboxylate (0.039 mL, 0.247 mmol). The reaction mixture stirred at room temperature under an inert atmosphere overnight. The residue was purified directly by silica gel chromatography (5/95—methanol/ethylacetate) to afford the product 159 (0.022 g, 26%): ¹H NMR (CDCl₃) δ 9.05 (d, 1H), 8.9 (d, 1H), 7.6 (dd, 1H), 7.5 (dd, 2H), 7.0 (t, 2H), 5.8 (s, 2H), 4.8 (d, 2H), 4.75 (m, 2H), 3.8 (d, 6H), 3.7 (s, 3H), 2.5 (m, 2H); ³¹P NMR (CDCl₃) δ 30.2; MS: 519 (M+1).

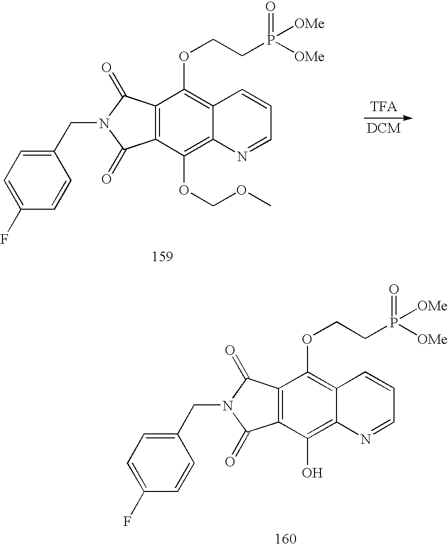

### Example 160

A solution of the phosphonate 159 (0.012 g, 0.024 mmol) in dichloromethane (0.863 mL) was treated with trifluoroacetic acid (0.018 mL, 0.240 mmol). The reaction mixture was stirred at room temperature under an inert atmosphere overnight. The volatiles were removed in vacuo with toluene. The solid was triturated in diethylether/hexane to afford the product 160 (0.0095 g, 84%) as a TFA salt: ¹H NMR (CDCl₃) δ 9.0 (d, 1H), 8.9 (d, 1H), 7.7 (dd, 1H), 7.5 (dd, 2H), 7.0 (t, 2H), 4.85 (d, 2H), 4.8 (m, 2H), 3.8 (d, 6H), 2.5 (m, 2H); ³¹P NMR (CDCl₃) δ 30.3; MS: 475 (M+1), 473 (M−1).

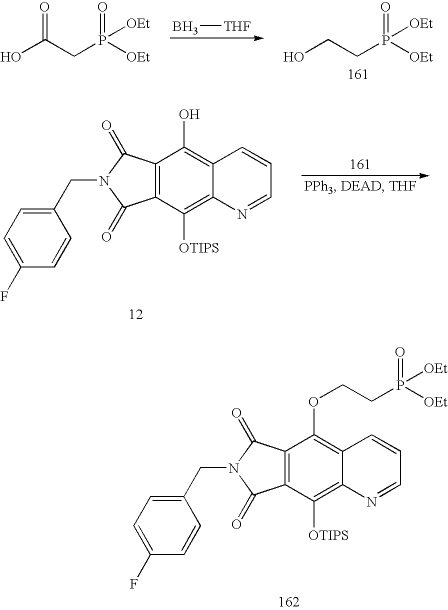

### Example 161

A solution of diethyl phosphonacetic acid (0.700 g, 3.57 mmol) dissolved in THF was cooled to 0° C. Borane-THF complex (7.14 mL) in 1M THF was added dropwise. The reaction mixture was stirred for 3 hours under an inert atmosphere then concentrated in vacuo. The residue was directly purified by silica gel chromatography (5/95—methanol/ethylacetate) to afford the product, diethyl hydroxyethyl phosphonate, 161 (0.583 g, 90%) as an oil: ¹H NMR (CDCl₃) δ 4.1 (m, 4H), 3.9 (m, 2H), 2.1 (m, 2H), 1.3 (t, 6H); ³¹P NMR (CDCl₃) δ 30.4; MS: 183 (M+1).

### Example 162

To a solution of phenol 12 (0.023 g, 0.046 mmol) dissolved in THF (0.24 mL) was added diethyl hydroxyethyl phosphonate 161 (0.025 g, 0.137 mmol), triphenylphosphine (0.030 g, 0.114 mmol), and diethyl azodicarboxylate (0.011 mL, 0.069 mmol). The reaction mixture stirred at room temperature under an inert atmosphere overnight. The residue was purified directly by silica gel chromatography (75/25—ethylacetate/hexane). The residue was purified again by silica gel chromatography (80/20 toluene/acetone) to afford the product 162 (0.032 g, 48%): ¹H NMR (CDCl₃) δ 8.9 (d, 1H), 8.8 (d, 1H), 7.6 (dd, 1H), 7.45 (dd, 2H), 7.0 (t, 2H), 4.8 (s, 2H), 4.7 (m, 2H), 4.15 (m, 4H), 2.5 (m, 2H), 1.5 (m, 3H), 1.3 (t, 6H), 1.2 (d, 18H); ³¹P NMR (CDCl₃) δ 27.6; MS: 659 (M+1).

### Example 163

A solution of the phosphonate 162 (0.012 g, 0.018 mmol) in dichloromethane (0.663 mL) was treated with trifluoroacetic acid (0.014 mL, 0.180 mmol). The reaction mixture was stirred at room temperature under an inert atmosphere overnight. The volatiles were removed in vacuo with toluene. The solid was triturated in diethylether/hexane to afford the product 163 (0.008 g, 89%) as a TFA salt: ¹H NMR (CDCl₃) δ 9.0 (dd, 1H), 8.9 (dd, 1H), 7.7 (dd, 1H), 7.5 (dd, 2H), 7.0 (t, 2H), 4.8 (s, 2H), 4.75 (m, 2H), 4.15 (m, 4H), 2.45 (m, 2H), 1.3 (t, 6H); ³¹P NMR (CDCl₃) δ 27.5; MS: 503 (M+1), 501 (M−1).

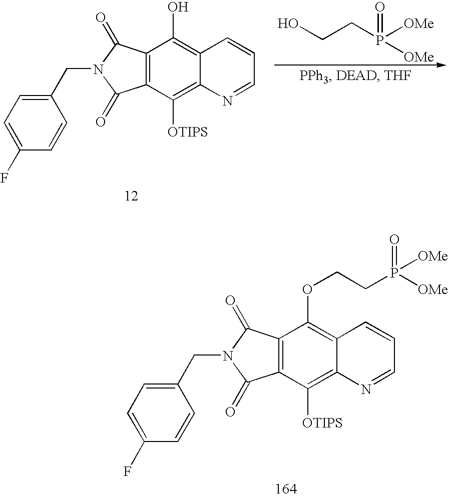

### Example 164

To a solution of phenol 12 (0.097 g, 0.196 mmol) dissolved in THF (1.02 mL) was added (2-hydroxyethyl)-phosphonic acid dimethyl ester (0.091 g, 0.589 mmol), triphenylphosphine (0.129 g, 0.491 mmol), and diethyl azodicarboxylate (0.046 mL, 0.295 mmol). The reaction mixture stirred at room temperature under an inert atmosphere overnight. The residue was purified directly by silica gel chromatography (85/15—ethylacetate/hexane) to afford a mixture of product 164 and triphenylphosphine oxide (0.160 g): ¹H NMR (CDCl₃) δ 8.95 (d, 1H), 8.75 (d, 1H), 7.7-7.4 (m, 12H), 7.0 (t, 2H), 4.8 (s, 2H), 4.7 (m, 2H), 3.8 (d, 6H), 2.5 (m, 2H), 1.5 (m, 3H), 1.2 (d, 18H); ³¹P NMR (CDCl₃) δ 30.5 (triphenylphosphine oxide), 29.3; MS: 631 (M+1).

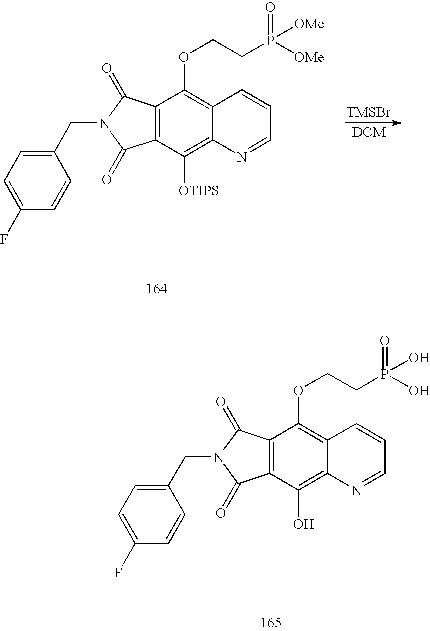

### Example 165

A solution of the phosphonate 164 (0.025 g, 0.040 mmol) in dichloromethane (0.397 mL) was treated with trimethylsilane bromide (0.0314 mL, 0.24 mmol). The reaction mixture was stirred at room temperature under an inert atmosphere overnight. The volatiles were removed in vacuo with methanol. The solid was washed with dichloromethane to afford the diacid 165 (0.0094 g, 53%): ¹H NMR (CD₃OD) δ 9.4 (dd, 1H), 9.1 (dd, 1H), 8.05 (dd, 1H), 7.5 (dd, 2H), 7.1 (t, 2H), 4.9 (s, 2H), 4.8 (m, 2H), 2.45 (m, 2H); ³¹P NMR (CD₃OD) δ 24.7; MS: 447 (M+1), 445 (M−1).

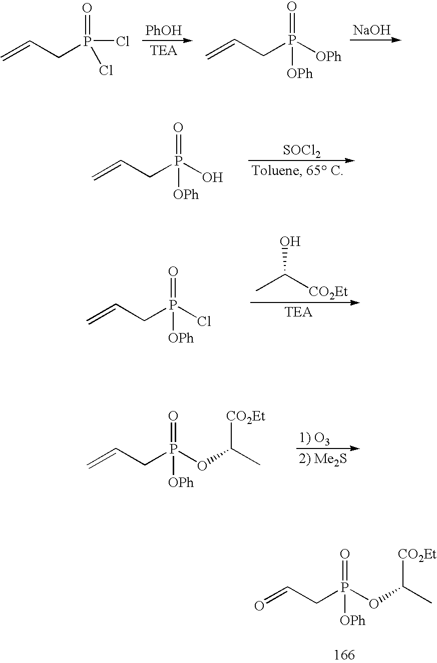

### Example 166

To a solution of allylphosphonic dichloride (4 g, 25.4 mmol) and phenol (5.2 g, 55.3 mmol) in CH₂Cl₂ (40 mL) at 0° C. was added triethylamine (TEA, 8.4 mL, 60 mmol). After stirring at room temperature for 1.5 h, the mixture was diluted with hexane-ethylacetate and washed with HCl (0.3 N) and water. The organic phase was dried over MgSO₄, filtered and concentrated under reduced pressure. The residue was filtered through a pad of silica gel (eluted with 2:1 hexane-ethyl acetate) to afford crude product diphenol allylphosphonate (7.8 g, containing the excessive phenol) as an oil which was used directly without any further purification. The crude material was dissolved in CH₃CN (60 mL), and NaOH (4.4N, 15 mL) was added at 0° C. The resulting mixture was stirred at room temperature for 3 h, then neutralized with acetic acid to pH=8 and concentrated under reduced pressure to remove most of the acetonitrile. The residue was dissolved in water (50 mL) and washed with CH₂Cl₂ (three 25 mL portions). The aqueous phase was acidified with concentrated HCl at 0° C. and extracted with ethyl acetate. The organic phase was dried over MgSO₄, filtered, evaporated and co-evaporated with toluene under reduced pressure to yield desired monophenol allylphosphonate (4.75 g. 95%) as an oil.

To a solution of monophenol allylphosphonate (4.75 g, 24 mmol) in toluene (30 mL) was added SOCl₂ (5 mL, 68 mmol) and DMF (0.05 mL). After stirring at 65° C. for 4 h, the reaction was complete as shown by ³¹P NMR. The reaction mixture was evaporated and co-evaporated with toluene under reduced pressure to give the mono chloride (5.5 g) as an oil. To a solution of the mono chloride in CH₂Cl₂ (25 mL) at 0° C. was added ethyl (S)-lactate (3.3 mL, 28.8 mmol), followed by TEA. The mixture was stirred at 0° C. for 5 min, then at room temperature for 1 h, and concentrated under reduced pressure. The residue was partitioned between ethylacetate and HCl (0.2N), the organic phase was washed with water, dried over MgSO₄, filtered and concentrated under reduced pressure. The residue was purified by chromatography on silica gel to afford the allyl monolactate (5.75 g, 80%) as an oil (2:1 mixture of two isomers): ¹H NMR (CDCl₃) δ 7.1-7.4 (m, 5H), 5.9 (m, 1H), 5.3 (m, 2H), 5.0 (m, 1H), 4.2 (m, 2H), 2.9 (m, 2H), 1.6; 1.4 (d, 3H), 1.25 (m, 3H); ³¹P NMR (CDCl₃) δ 25.4, 23.9.

A solution of the allyl monolactate (2.5 g, 8.38 mmol) in CH₂Cl₂ (30 mL) was bubbled with ozone air at −78° C. until the solution became blue, then bubbled with nitrogen until the blue color disappeared. Methyl sulfide (3 mL) was added at −78° C. The mixture was warmed up to room temperature, stirred for 16 h and concentrated under reduced pressure to give desired aldehyde 166 (3.2 g, as a 1:1 mixture of DMSO): ¹H NMR (CDCl₃) δ 9.8 (m, 1H), 7.1-7.4 (m, 5H), 5.0 (m, 1H), 4.2 (m, 2H), 3.4 (m, 2H), 1.6; 1.4 (d, 3H), 1.25 (m, 3H). ³¹P NMR (CDCl₃) δ 17.7, 15.4.

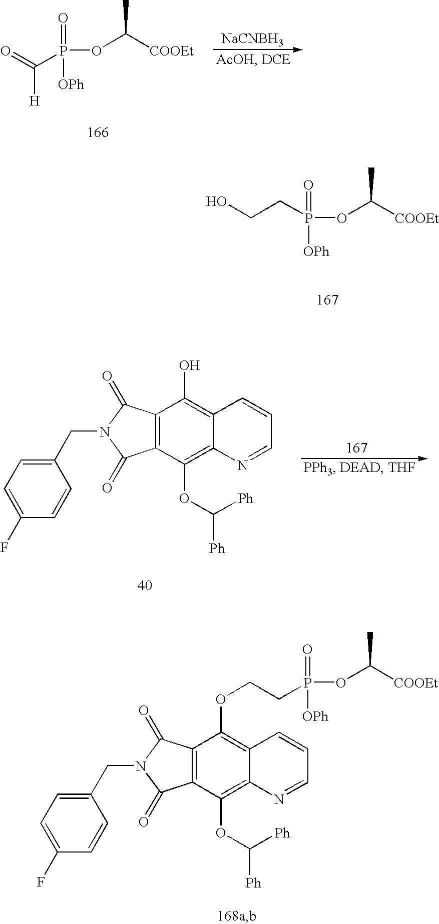

### Example 167

To a solution of 2-[(2-oxo-ethyl)-phenoxy-phosphinoyloxy]-propionic acid ethyl ester; aldehyde 166 (0.082 g, 0.218 mmol) in a 1:1 mixture of DMSO and 1,2-dichloroethane was added acetic acid (0.050 mL, 0.870 mmol) then sodium cyanoborohydride (0.027 g, 0.435 mmol). The reaction mixture stirred at room temperature for three hours under an inert atmosphere. Saturated NaHCO₃ was added to the reaction mixture and was stirred for five more minutes. The mixture was concentrated in vacuo to remove most of the dichloroethane. Brine was added and then the crude product was extracted into ethylacetate. The organic phase was dried (MgSO₄) and concentrated. The residue was purified by silica gel chromatography (5/95—methanol/dichloromethane) to afford the product 167 (0.047 g, 73%), an oil as a mixture of two diastereomers: ¹H NMR (CDCl₃) δ 7.1-7.4 (m, 5H), 5.1 (m, 1H), 4.25 (m, 2H), 4.1 (m, 2H), 2.3 (m, 4H), 1.6 & 1.4 (d, 3H), 1.25 (m, 3H); ³¹P NMR (CDCl₃) δ 29.0, 26.8.

### Example 168

To a solution of phenol 40 (0.033 g, 0.065 mmol) dissolved in THF (0.34 mL) was added ethyl-lactate phosphonate alcohol 167 (0.029 g, 0.097 mmol), triphenylphosphine (0.043 g, 0.162 mmol), and diethyl azodicarboxylate (0.015 mL, 0.097 mmol). The reaction mixture stirred at room temperature under an inert atmosphere overnight. The residue was purified directly by silica gel chromatography (50/50—ethylacetate/hexane) to afford the product 168 (0.027 g, 53%). Separation of the diastereomers by chromatography allowed for characterization of 168a (0.016 g): ¹H NMR (CDCl₃) δ 9.1 (dd, 1H), 8.8 (dd, 1H), 7.9 (s, 1H), 7.6 (m, 4H), 7.55 (m, 1H), 7.4 (dd, 2H), 7.1-7.4 (m, 11H), 7.0 (t, 2H), 5.0 (m, 1H), 4.9 (s, 2H), 4.8 (m, 2H), 4.1 (q, 3H), 2.75 (m, 2H), 1.4 (d, 3H), 1.2 (t, 3H); ³¹P NMR (CDCl₃) δ 26.05; MS: 790 (M+1)− and 168b (0.011 g): ¹H NMR (CDCl₃) δ 9.1 (dd, 1H), 8.8 (dd, 1H), 7.95 (s, 1H), 7.6 (m, 4H), 7.55 (m, 1H), 7.40 (dd, 2H), 7.1-7.4 (m, 11H), 7.05 (t, 2H), 5.05 (m, 1H), 4.85 (s, 2H), 4.8 (m, 2H), 4.15 (q, 3H), 2.7 (m, 2H), 1.55 (d, 3H), 1.2 (t, 3H); ³¹P NMR (CDCl₃) δ 24.37; MS: 790 (M+1)

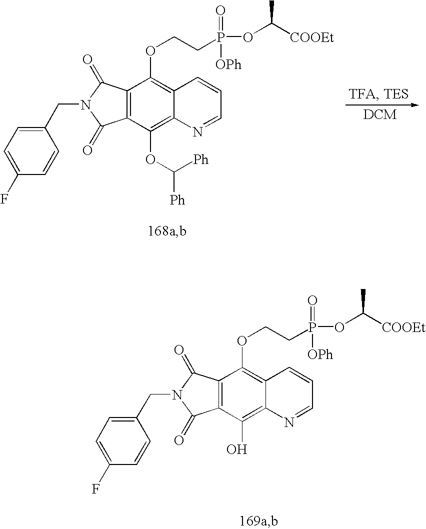

### Example 169a

A solution of the phosphonate 168a (0.013 g, 0.0165 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 169a (0.008 g, 80%) as a TFA salt: ¹H NMR (CDCl₃) δ 8.95 (dd, 1H), 8.9 (dd, 1H), 7.6 (m, 1H), 7.5 (dd, 2H), 7.1-7.4 (m, 5H), 7.0 (t, 2H), 5.0 (m, 1H), 5.0 (m, 2H), 4.85 (s, 2H), 4.15 (q, 3H), 2.8 (m, 2H), 1.4 (d, 3H), 1.25 (t, 3H); ³¹P NMR (CDCl₃) δ 26.13; MS: 623 (M+1), 621 (M−1).

### Example 169b

A solution of the phosphonate 168b (0.011 g, 0.014 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 169b (0.005 g, 60%) as a TFA salt: ¹H NMR (CDCl₃) δ 8.95 (dd, 1H), 8.9 (dd, 1H), 7.65 (m, 1H), 7.5 (dd, 2H), 7.1-7.4 (m, 5H), 7.0 (t, 2H), 5.1 (m, 2H), 4.9 (m, 1H), 4.85 (s, 2H), 4.15 (q, 3H), 2.7 (m, 2H), 1.55 (d, 3H), 1.2 (t, 3H); ³¹P NMR (CDCl₃) δ 24.44; MS: 623 (M+1), 621 (M−1).

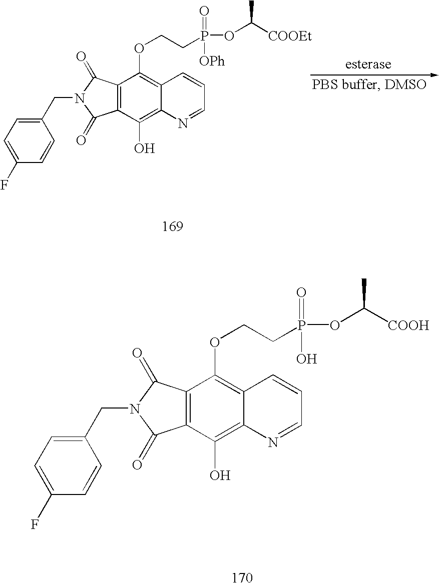

### Example 170

A solution of ethyl-lactate phosphonate 169 (0.021 g, 0.034 mmol) in DMSO (0.675 mL) and phosphate buffer saline (3.38 ml) was heated to 40° C. The reaction mixture was treated with esterase—from porcine liver (0.200 mL) and stirred overnight. Another equivalent of esterase was added the following day and the mixture stirred another day. The mixture was concentrated and purified by reversed phase HPLC to afford the product 170 (0.008 g, 46%) as a solid: ¹H NMR (CD₃OD) δ 8.95 (dd, 1H), 8.9 (dd, 1H), 7.75 (m, 1H), 7.45 (dd, 2H), 7.05 (t, 2H), 4.9 (s, 2H), 4.85 (m, 3H), 2.5 (m, 2H), 1.5 (d, 3H); ³¹P NMR (CD₃OD) δ 26.26; MS: 519 (M+1), 517 (M−1).

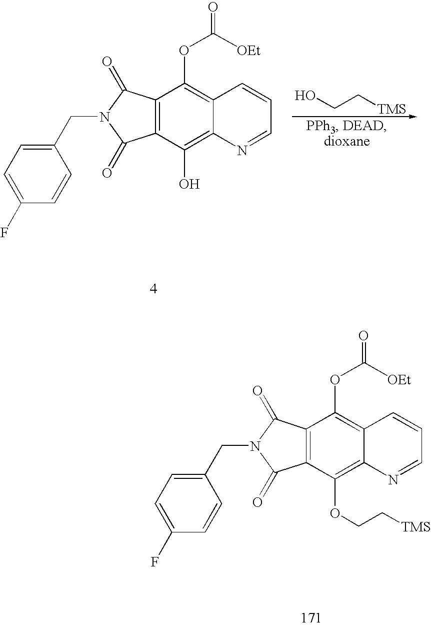

### Example 171

To a solution of phenol 4 (1.14 g, 2.79 mmol) dissolved in dioxane (27.9 mL) was added 2-(trimethylsilyl)-ethanol (0.600 mL, 4.18 mmol), triphenylphosphine (1.46 g, 5.57 mmol), and diethyl azodicarboxylate (0.88 mL, 5.57 mmol). The reaction mixture stirred at room temperature under an inert atmosphere overnight. The residue was purified directly by silica gel chromatography (30/70—ethylacetate/hexane) to afford the product 171 (0.240 g, 67%): ¹H NMR (CDCl₃) δ 9.1 (dd, 1H), 8.5 (dd, 1H), 7.65 (dd, 1H), 7.45 (dd, 2H), 7.0 (t, 2H), 4.9 (m, 2H), 4.8 (s, 2H), 4.45 (q, 2H), 1.5 (t, 3H), 1.4 (m, 2H), 0.1 (s, 9H); MS: 510 (M+1).

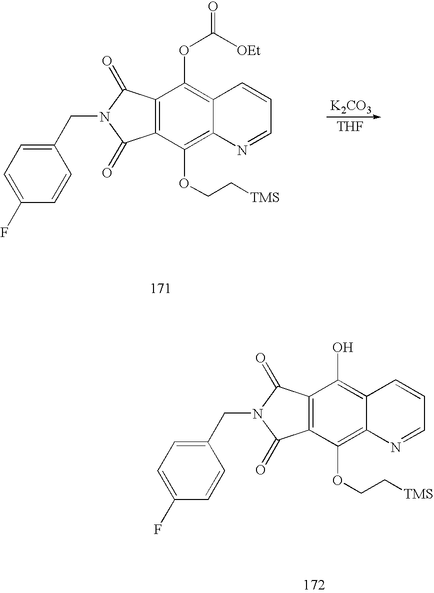

### Example 172

To the ethyl carbonate 171 (0.716 g, 1.4 mmol) in THF (70.2 mL) was added a solution (45 mL) of K₂CO₃ (1.94 g, 14 mmol) in water and 4-dimethylaminopyridine (0.035 g, 0.281 mmol). The yellow solution was stirred at room temperature under an inert atmosphere overnight. Most of THF was removed in vacuo and the remaining solution was diluted with dichloromethane, washed with 1N HCl and brine, then dried (MgSO₄) and concentrated. The crude product was triturated in diethylether/hexane to afford the yellow solid product 172 (0.428 g, 70%): ¹H NMR (CDCl₃) δ 9.1 (dd, 1H), 8.65 (dd, 1H), 7.6 (dd, 1H), 7.5 (dd, 2H), 7.0 (t, 2H), 4.85 (s, 2H), 4.85 (m, 2H), 1.35 (m, 2H), 0.1 (s, 9H); MS: 438 (M+1).

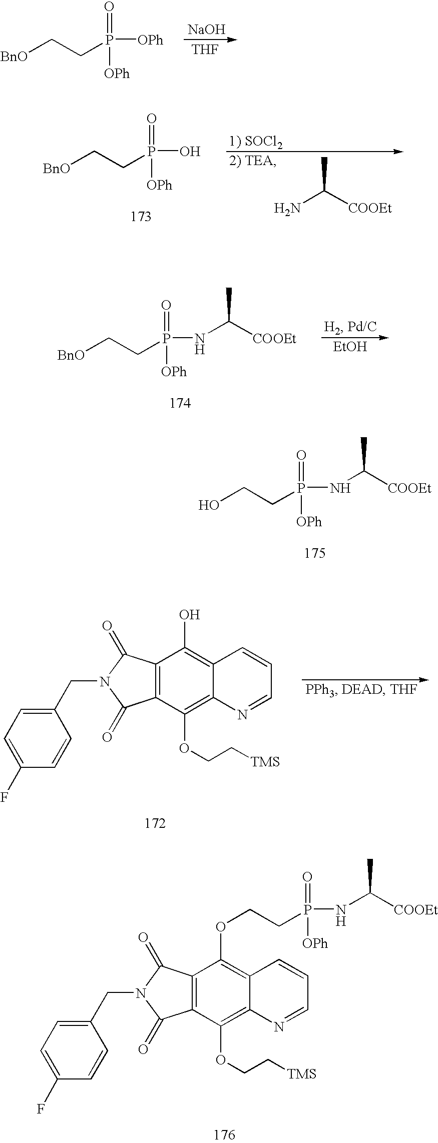

### Example 173

To a solution of (2-benzyloxy-ethyl)-phosphonic acid dibenzyl ester (0.200 g, 0.543 mmol) in THF was added a solution of NaOH (1.36 mL, 1M) in water. The reaction mixture was stirred at room temperature for 3 hours. Most of THF was removed in vacuo and the residue was dissolved in water. The aqueous solution was washed with ethylacetate three times then acidified with 1N HCl (to pH=1) then extracted with ethylacetate. The organic phase was dried (MgSO₄), concentrated and co-evaporated with toluene in vacuo to afford the mono-acid, (2-benzyloxy-ethyl)-phosphonic acid monobenzyl ester, 173 (0.160 g. 100%) as an oil with no further purification: ¹H NMR (CDCl₃) δ 9.25 (bs, 1H), 7.4-7.1 (m, 10H), 4.5 (s, 2H), 3.8 (m, 2H), 2.25 (m, 2H); ³¹P NMR (CDCl₃) δ 28.63.

### Example 174

To a solution of the mono-acid 173 (0.160 g, 0.576 mmol) dissolved in acetonitrile (3.84 mL) was added thionyl chloride (0.42 mL, 5.76 mmol). The reaction mixture was heated to 70° C. and stirred for 3 hours at which point the reaction was completed as shown by ³¹P NMR (CDCl₃) δ 36.7. The reaction mixture was concentrated as such to afford the intermediate mono-chloridate as an oil which was immediately dissolved in dichloromethane (2.88 mL) and treated with triethylamine (0.321 mL, 2.30 mmol). The reaction mixture was cooled to 0° C. and L-alanine ethyl ester (0.265 g, 1.73 mmol) was added. The mixture was stirred overnight at room temperature under an inert atmosphere and then was concentrated in vacuo. The residue was partitioned between ethylacetate and saturated NH₄Cl, and the organic phase was washed with brine, dried (MgSO₄) then concentrated in vacuo. The residue was purified by chromatography on silica gel washed with methanol prior to use (1/1—ethylacetate/hexane) to afford the amidate 174 (0.095 g, 45%) as an oil with a 1:1.2 mixture of diastereomers: ¹H NMR (CDCl₃) δ 7.1-7.4 (m, 10H), 4.6 (s, 2H), 4.1 (q, 2H), 3.8 (m, 2H), 3.65 (m, 1H), 2.3 (m, 2H), 1.3 & 1.2 (d, 3H), 1.25 (t, 3H); ³¹P NMR (CDCl₃) δ 29.51, 28.70.

### Example 1756

To a solution of the amidate 174 (0.095 g, 0.243 mmol) dissolved in ethanol (4.9 mL) was added palladium (on carbon). The reaction was purged under a vacuum then submitted to hydrogen gas (via balloon attached to the reaction vessel). After several purges between gas and vacuum the reaction mixture was stirred at room temperature for 4 hours. The mixture was filtered with Celite and concentrated in vacuo to afford the alcohol 175 (0.74 g, 100%) as an oil with a 1:1.2 mixture of diastereomers without further purification: ¹H NMR (CDCl₃) δ 7.4-7.1 (m, 5H), 4.15 (m, 2H), 3.7 (q, 2H), 3.5 (m, 1H), 2.2 (m, 2H), 1.35 & 1.25 (d, 3H), 1.25 (m, 3H); ³¹P NMR (CDCl₃) δ 30.82, 30.54.

### Example 176

To a solution of phenol 172 (0.073 g, 0.167 mmol) dissolved in THF (1.67 mL) was added the alcohol 175 (0.075 g, 0.25 mmol), triphenylphosphine (0.087 g, 0.33 mmol), and diethyl azodicarboxylate (0.042 mL, 0.33 mmol). The reaction mixture stirred at room temperature under an inert atmosphere overnight. The residue was purified directly by chromatography on silica gel washed with methanol prior to use (80/20—toluene/acetone) to afford the product 176 (0.065 g, 54%) with a 1:1.2 mixture of diastereomers: ¹H NMR (CDCl₃) δ 9.1 (dd, 1H), 8.8 (dd, 1H), 7.6 (dd, 1H), 7.5 (dd, 2H), 7.4-7.1 (m, 5H), 7.0 (t, 2H), 4.85 (s, 2H), 4.85-4.7 (m, 4H), 4.2 (q, 1H), 4.15 (m, 2H), 4.0-3.8 (m, 1H), 2.65 (m, 2H), 1.4 & 1.25 (d, 3H), 1.3 (m, 2H), 1.2 (m, 3H), 0.10 (s, 9H); ³¹P NMR (CDCl₃) δ 27.84, 26.96; MS: 722 (M+1).

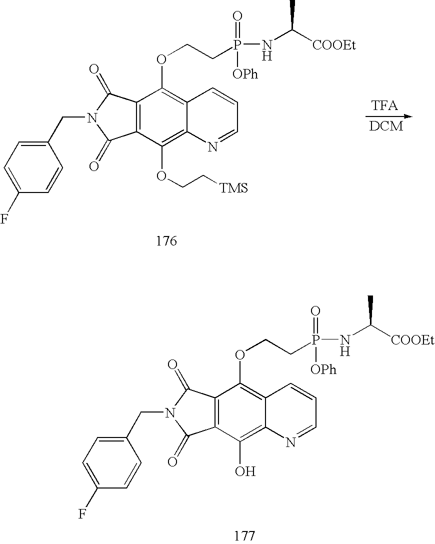

### Example 177

A solution of the phosphonate 176 (0.030 g, 0.042 mmol) in dichloromethane (0.832 mL) was treated with trifluoroacetic acid (0.064 mL, 0.84 mmol). The reaction mixture was stirred at room temperature under an inert atmosphere for 45 minutes. The volatiles were removed in vacuo with toluene. The solid was triturated in diethylether/hexane to afford the product 177 (0.022 g, 85%) as a TFA salt with a 1:1.2 mixture of diastereomers: ¹H NMR (CDCl₃) δ 9.0 (dd, 1H), 8.85 (dd, 1H), 7.65 (dd, 1H), 7.5 (dd, 2H), 7.4-7.1 (m, 5H), 7.0 (t, 2H), 4.85 (s, 2H), 4.85 (m, 2H), 4.15 (m, 1H), 4.15 (m, 1H), 4.1 (m, 2H), 3.8 (m, 1H), 2.65 (m, 2H), 1.35 & 1.30 (d, 3H), 1.2 (m, 3H); ³¹P NMR (CDCl₃) δ 27.86, 27.05; MS: 622 (M+1), 620 (M−1).

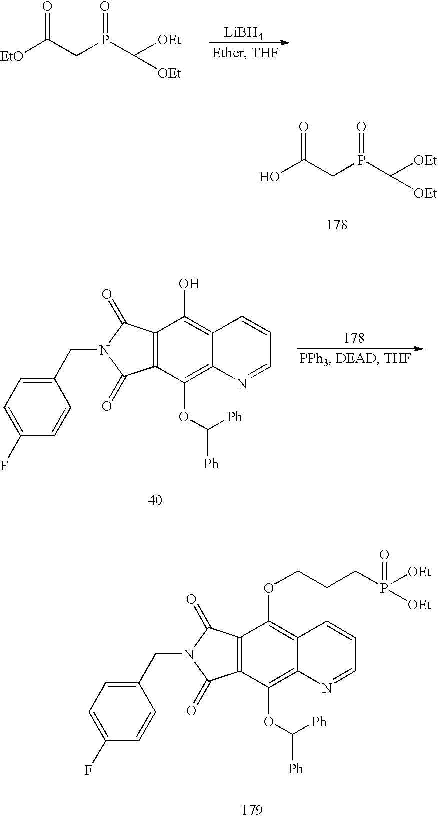

### Example 178

A solution of (2-ethoxy-ethyl)-phosphonic acid diethyl ester (0.500 g, 2.1 mmol) in ether (8.5 mL) and THF (1.5 mL) was treated with lithium borohydride. The reaction mixture stirred at room temperature for 1 hour and was then concentrated in vacuo. The crude mixture was partitioned between dichloromethane and water. The organic phase was washed with saturated NaHCO₃ and brine, dried (MgSO₄), then concentrated in vacuo. The residue was purified by silica gel chromatography (5/95—methanol/dichloromethane) to afford (3-hydroxy-propyl)-phosphonic acid diethyl ester 178 (0.100 g, 24%) as an oil: ¹H NMR (CDCl₃) δ 4.1 (m, 4H), 3.7 (m, 2H), 2.95 (bs, 1H), 1.85 (m, 4H), 1.30 (t, 3H); ³¹P NMR (CDCl₃) δ 33.26; MS: 197 (M+1).

### Example 179

To a solution of phenol 40 (0.023 g, 0.046 mmol) dissolved in THF (0.45 mL) was added the alcohol 178 (0.013 g, 0.068 mmol), triphenylphosphine (0.024 g, 0.091 mmol), and diethyl azodicarboxylate (0.014 mL, 0.091 mmol). The reaction mixture stirred at room temperature under an inert atmosphere overnight. The residue was purified directly by silica gel chromatography (90/10—ethylacetate/hexane) to afford the product 179 (0.024 g, 76%): ¹H NMR (CDCl₃) δ 9.1 (dd, 1H), 8.6 (dd, 1H), 7.9 (dd, 1H), 7.6 (m, 6H), 7.4 (dd, 2H), 7.2 (m, 6H), 7.0 (t, 2H), 4.8 (s, 2H), 4.5 (t, 2H), 4.15 (m, 2H), 2.2 (m, 2H), 2.0 (m, 2H), 1.35 (t, 3H); ³¹P NMR (CDCl₃) δ 31.48; MS: 684 (M+1).

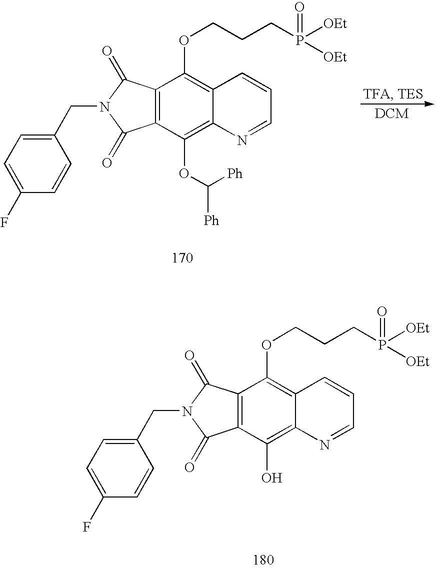

### Example 180

A solution of the phosphonate 179 (0.028 g, 0.041 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 180 (0.020 g, 95%) as a TFA salt: ¹H NMR (CDCl₃) δ 9.0 (dd, 1H), 8.7 (dd, 1H), 7.65 (dd, 1H), 7.5 (dd, 2H), 7.0 (t, 2H), 4.85 (s, 2H), 4.6 (t, 2H), 4.15 (m, 2H), 2.25 (m, 2H), 2.05 (m, 2H), 1.35 (t, 3H); ³¹P NMR (CDCl₃) δ 31.45; MS: 517 (M+1), 516 (M−1).

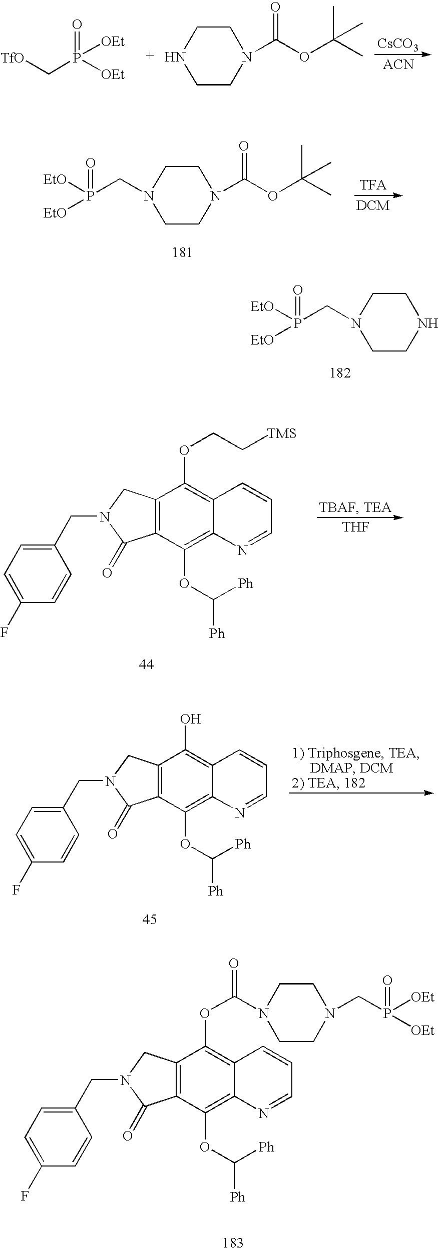

### Example 181

To a solution of 1-BOC-piperazine (0.200 g, 1.08 mmol) in acetonitrile (10.4 mL) was added CsCO₃ (1.05 g, 3.23 mmol) and then cooled to 0° C. Trifluoromethanesulfonic acid diethoxyphosphorylmethyl ester (0.387 g, 1.29 mmol) dissolved in acetonitrile (5 mL) was added in a dropwise manner. The reaction mixture was stirred at room temperature for 1 hour upon which it was concentrated in vacuo. The reaction mixture was taken into ethylacetate then washed with saturated NH₄Cl and brine, dried (MgSO₄), then concentrated in vacuo. The residue was purified using silica gel chromatography (3/97—methanol/dichloromethane) to afford the product 181 (0.310 g, 86%) as an oil: ¹H NMR (CDCl₃) δ 4.15 (m, 4H), 3.45 (t, 4H), 2.8 (d, 2H), 2.6 (m, 4H), 1.45 (s, 9H), 1.35 (t, 6H); ³¹P NMR (CDCl₃) δ 24.03; MS: 337 (M+1).

### Example 182

A solution of the BOC protected piperazine linker phosphonate 181 (0.310 g, 0.923 mmol) in dichloromethane (6.15 mL) was treated with trifluoroacetic acid (0.711 mL, 9.23 mmol). The reaction mixture was stirred at room temperature under an inert atmosphere overnight. The volatiles were removed in vacuo with toluene to afford the free piperazine linker phosphonate 182 (0.323 g, 100%) as a TFA salt: ¹H NMR (CDCl₃) δ 11.0 (bs, 1H), 4.2 (m, 4H), 3.45 (t, 4H), 3.35 (m, 4H), 3.2 (d, 2H), 1.4 (t, 6H); ³¹P NMR (CDCl₃) δ 19.16; MS: 237 (M+1).

### Example 183

A solution of the phenol intermediate 45 (0.044 mmol) in dichloromethane (0.441 mL) was treated with triethylamine (0.025 mL, 0.176 mmol) and cat. 4-dimethylaminopyridine. The reaction mixture was cooled to 0° C. then triphosgene (0.026 g, 0.088 mmol) in a 1M solution of dichloromethane was added. The mixture stirred at room temperature under an inert atmosphere for 2 hours, then the free piperazine linker phosphonate 182 (0.046 g, 0.132 mmol) in a 1M solution of dichloromethane treated with triethylamine (0.025 mL, 0.176 mmol) was added, and the mixture was stirred overnight. The mixture was partitioned between dichloromethane and water. The organic phase was washed with saturated NH₄Cl and brine, dried (MgSO₄), and concentrated in vacuo. The residue was purified by silica gel chromatography (3/97—methanol/dichloromethane) to afford the product 183 (0.016 g, 64%): ¹H NMR (CDCl₃) δ 9.05 (dd, 1H), 8.1 (dd, 1H), 8.0 (s, 1H), 7.75 (d, 4H), 7.5 (dd, 1H), 7.4-7.m, 8H), 7.05 (t, 2H), 4.8 (s, 2H), 4.2 (s, 2H), 4.15 (m, 4H), 3.75 (m, 2H), 3.6 (m, 2H), 2.85 (d, 2H), 2.8 (m, 2H), 2.75 (m, 2H), 1.35 (t, 6H); ³¹P NMR (CDCl₃) δ 23.57; MS: 753 (M+1).

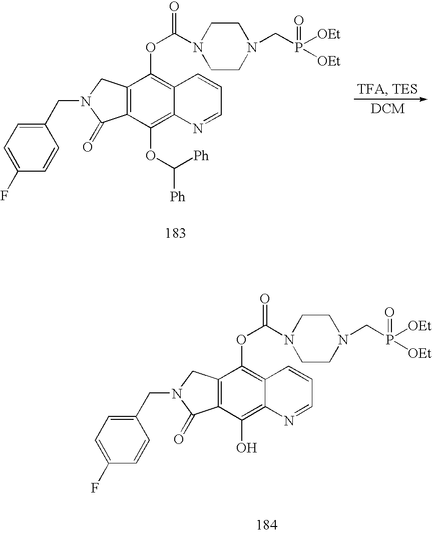

### Example 184

A solution of the phosphonate 183 (0.016 g, 0.021 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 184 (0.0125 g, 100%) as a TFA salt: ¹H NMR (CDCl₃) δ 9.0 (dd, 1H), 8.2 (dd, 1H), 7.6 (dd, 1H), 7.3 (m, 2H), 7.05 (t, 2H), 4.75 (s, 2H), 4.35 (s, 2H), 4.2 (m, 4H), 3.95 (m, 2H), 3.75 (m, 2H), 3.2 (d, 2H), 3.2 (m, 2H), 3.1 (m, 2H), 1.4 (t, 6H); ³¹P NMR (CDCl₃) δ 19.93; MS: 587 (M+1), 585 (M−1).

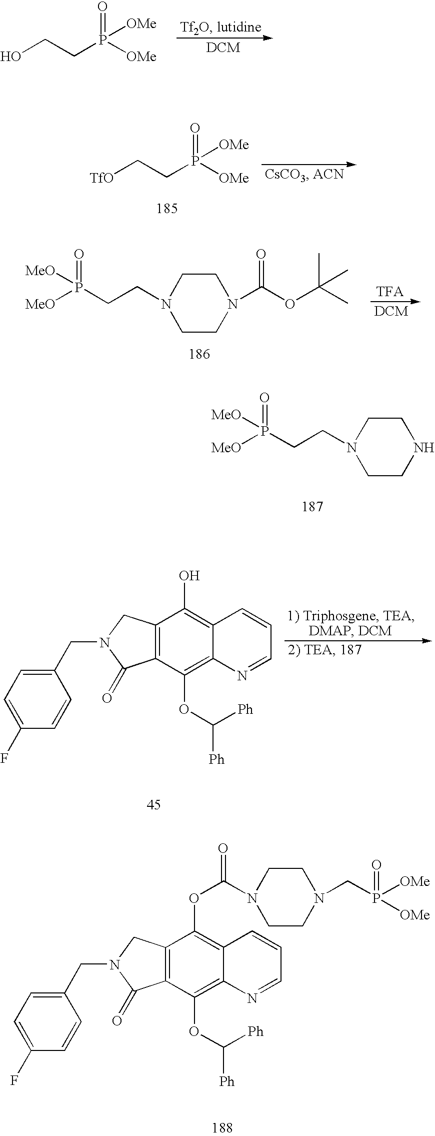

### Example 185

To a solution of (2-hydroxy-ethyl)-phosphonic acid dimethyl ester (0.250 g, 1.62 mmol) in dichloromethane (4 mL) was added 2,6-lutidine (0.284 mL, 2.44 mmol). The reaction mixture was cooled to −40° C. and trifluoromethanesulfonic anhydride (0.355 mL, 2.11 mmol) was added. The mixture stirred in the cold bath under an inert atmosphere for 2 hours at which point the reaction was completed as shown by ³¹P NMR (CDCl₃) δ 25.7. The mixture was partitioned between dichloromethane and water both cooled by an ice-water bath. The organic phase was washed with brine, dried (MgSO₄), and concentrated in vacuo to afford trifluoromethanesulfonic acid dimethoxy-phosphoryl-2-ethyl ester 185 as an oil which was immediately carried forward with no further purification or characterization.

### Example 186

To a solution of 1-BOC-piperazine (0.252 g, 1.35 mmol) in acetonitrile (14.3 mL) was added CsCO₃ (1.32 g, 4.06 mmol) and then cooled to 0° C. Trifluoromethanesulfonic acid dimethoxy-phosphoryl-2-ethyl ester 185 (0.464 g, 1.62 mmol) dissolved in acetonitrile (5 mL) was added in a dropwise manner. The reaction mixture was stirred at room temperature overnight upon which it was concentrated in vacuo. The reaction mixture was taken into ethylacetate then washed with saturated NH₄Cl and brine, dried (MgSO₄), then concentrated in vacuo. The residue was purified using silica gel chromatography (5/95—methanol/dichloromethane) to afford the BOC protected piperazine linker phosphonate 186 (0.162 g, 31% over 2 steps) as an oil: ¹H NMR (CDCl₃) δ 3.75 (d, 6H), 3.4 (m, 4H), 2.65 (m, 2H), 2.4 (m, 4H), 1.95 (m, 2H), 1.45 (s, 9H); ³¹P NMR (CDCl₃) δ 33.06; MS: 323 (M+1).

### Example 187

A solution of the BOC protected piperazine linker phosphonate 186 (0.162 g, 0.503 mmol) in dichloromethane (3.35 mL) was treated with trifluoroacetic acid (0.388 mL, 5.03 mmol). The reaction mixture was stirred at room temperature under an inert atmosphere overnight. The volatiles were removed in vacuo with toluene to afford the free piperazine linker phosphonate 187 (0.169 g, 100%) as a TFA salt: ¹H NMR (CD₃OD) δ 3.8 (d, 6H), 3.45 (m, 4H), 3.2 (m, 4H), 3.15 (m, 2H), 2.3 (m, 2H); ³¹P NMR (CDCl₃) δ 30.92; MS: 223 (M+1).

### Example 188

A solution of the phenol intermediate 45 (0.046 mmol) in dichloromethane (0.458 mL) was treated with triethylamine (0.026 mL, 0.183 mmol) and a catalytic amount of 4-dimethylaminopyridine. The reaction mixture was cooled to 0° C. then triphosgene (0.027 g, 0.092 mmol) in a 1M solution of dichloromethane was added. The mixture was stirred at room temperature under an inert atmosphere for 2 hours, then the free piperazine linker phosphonate 187 (0.046 g, 0.137 mmol) in a 1M solution of dichloromethane treated with triethylamine (0.026 mL, 0.183 mmol) was added dropwise. The mixture was stirred overnight and then partitioned between dichloromethane and water. The organic phase was washed with saturated NH₄Cl and brine, dried (MgSO₄), and concentrated in vacuo. The residue was purified by silica gel chromatography (8/92—methanol/ethylacetate) to afford the product 188 (0.019 g, 56%): ¹H NMR (CDCl₃) δ 9.05 (dd, 1H), 8.1 (dd, 1H), 8.05 (s, 1H), 7.75 (m, 4H), 7.5 (dd, 1H), 7.4-7.1 (m, 8H), 7.1 (t, 2H), 4.8 (s, 2H), 4.2 (s, 2H), 3.8 (d, 6H), 3.6 (m, 4H), 2.75 (m, 2H), 2.55 (m, 4H), 2.1 (m, 2H); ³¹P NMR (CDCl₃) δ 32.65; MS: 739 (M+1).

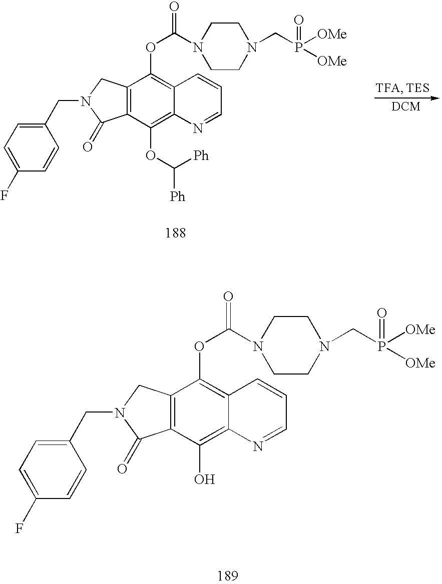

### Example 189

A solution of the phosphonate 188 (0.019 g, 0.026 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 189 (0.013 g, 74%) as a TFA salt: ¹H NMR (CDCl₃) δ 8.9 (dd, 1H), 8.15 (dd, 1H), 7.55 (dd, 1H), 7.35 (m, 2H), 7.05 (t, 2H), 4.75 (s, 2H), 4.35 (s, 2H), 4.2 (m, 2H), 3.95 (m, 2H), 3.8 (d, 6H), 3.4 (m, 4H), 3.35 (m, 2H), 2.4 (m, 2H); ³¹P NMR (CDCl₃) δ 27.31; MS: 573 (M+1).

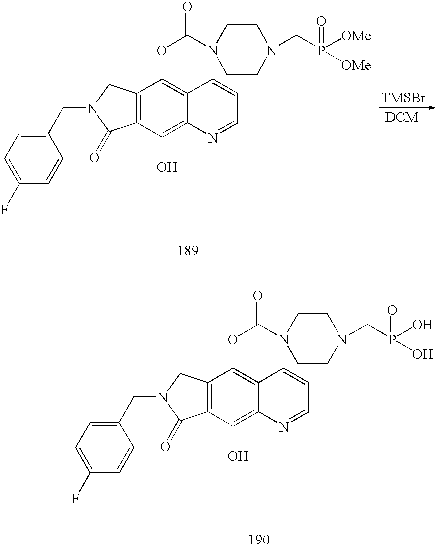

### Example 190

A solution of the phosphonate 189 (0.006 g, 0.009 mmol) in dichloromethane (0.088 mL) was treated with trimethylsilane bromide (0.007 mL, 0.053 mmol). The reaction mixture was stirred at room temperature overnight under an inert atmosphere. The volatiles were removed in vacuo with methanol. The solid was washed with dichloromethane to afford the diacid 190 (0.006 g, 100%): ¹H NMR (CD₃OD) δ 9.3 (dd, 1H), 9.2 (dd, 1H), 8.2 (dd, 1H), 7.4 (m, 2H), 7.1 (t, 2H), 4.8 (s, 2H), 4.6 (s, 2H), 3.6-3.2 (m, 10H), 2.35 (m, 2H); ³¹P NMR (CD₃OD) δ 21.43; MS: 545 (M+1), 543 (M−1).

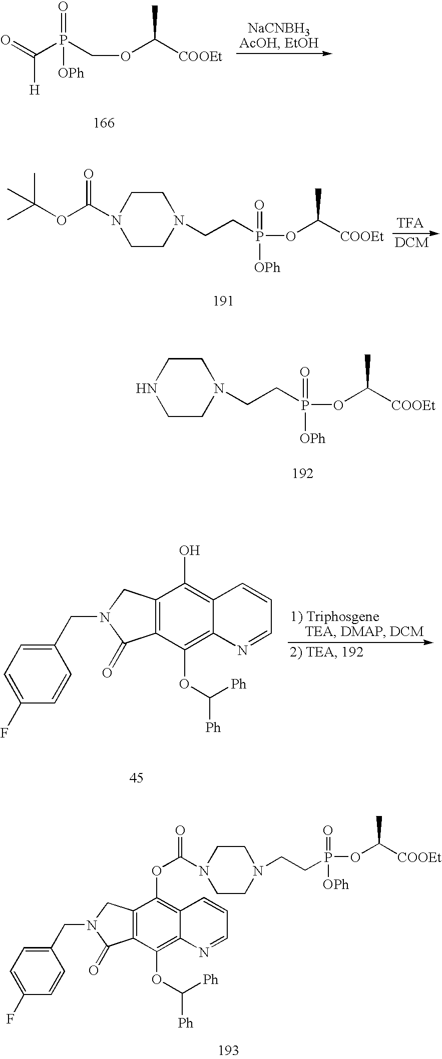

### Example 191

To a solution of 2-[(2-oxo-ethyl)-phenoxy-phosphinoyloxy]-propionic acid ethyl ester, aldehyde 166, as a 1:1 mixture of DMSO (0.050 g, 0.167 mmol) and 1-BOC-piperazine (0.034 g, 0.183 mmol) dissolved in ethanol (1.67 mL) was added acetic acid (0.038 mL, 0.667 mmol). The reaction mixture was stirred at room temperature for 2.5 hours then sodium cyanoborohydride (0.021 g, 0.333 mmol) was added. The reaction mixture stirred at room temperature overnight. Saturated NaHCO₃ was added to the reaction mixture and was stirred for five more minutes. The mixture was concentrated in vacuo to remove most of the ethanol. Brine was added and then the crude product was extracted into ethylacetate. The organic phase was dried (MgSO₄) and concentrated. The residue was purified by silica gel chromatography (5/95—methanol/dichloromethane) to afford the product 191 (0.050 g, 64%), an oil as a mixture of diastereomers: ¹H NMR (CDCl₃) δ 7.4-7.1 (m, 5H), 5.0 (m, 1H), 4.2 (m, 2H), 3.4 (m, 4H), 2.8 (m, 2H), 2.4 (m, 4H), 2.2 (m, 2H), 1.6 & 1.35 (d, 3H), 1.4 (s, 9H), 1.2 (t, 3H); ³¹P NMR (CDCl₃) δ 28.83, 27.18; MS: 471 (M+1).

Alternatively, a solution of 2-[(2-oxo-ethyl)-phenoxy-phosphinoyloxy]-propionic acid ethyl ester 166, as a 1:1 mixture with DMSO (0.500 g, 1.67 mmol), and piperazine-1-carboxylic acid tert-butyl ester (1-BOC-piperazine, 0.340 g, 1.83 mmol) dissolved in ethanol (1.67 mL) was added 4 A molecular sieves (0.300 g) and acetic acid (0.400 mL, 6.8 mmol). The reaction mixture was stirred at room temperature for 1.5 hours then sodium cyanoborohydride (0.212 g, 3.33 mmol) was added. The reaction mixture stirred at room temperature for 3 hours and was concentrated in vacuo then redissolved in chloroform. The mixture was washed with saturated NaHCO₃ and brine, dried (NaSO₄), filtered and concentrated. The residue was treated with diethyl ether. Solid precipitate was filtered off, and the filtrate was concentrated to afford 4-{2-[(1-Ethoxycarbonyl-ethoxy)-phenoxy-phosphoryl]-ethyl}-piperazine-1-carboxylic acid tert-butyl ester 191 (0.600 g, 77%) as an oil (mixture of two diastereomers).

### Example 192

A solution of 4-{2-[(1-ethoxycarbonyl-ethoxy)-phenoxy-phosphoryl]-ethyl}-piperazine-1-carboxylic acid tert-butyl ester 191 (0.050 g, 0.106 mmol) in dichloromethane (0.709 mL) was treated with trifluoroacetic acid (0.082 mL, 1.06 mmol). The reaction mixture was stirred at room temperature under an inert atmosphere for 4 hours. The volatiles were removed in vacuo with toluene to afford the free piperazine linker phosphonate 192 (0.051 g, 100%) as a TFA salt (mixture of two diastereomers): ¹H NMR (CDCl₃) δ 10.8 (bs, 1H), 7.5-7.1 (m, 5H), 5.0 (m, 1H), 4.2 (m, 4H), 3.7 (m, 8H), 2.65 (m, 2H), 1.6 & 1.4 (d, 3H), 1.25 (t, 3H); ³¹P NMR (CDCl₃) δ 25.58, 20.86; MS: 371 (M+1).

Alternatively a solution of 4-{2-[(1-ethoxycarbonyl-ethoxy)-phenoxy-phosphoryl]-ethyl}-piperazine-1-carboxylic acid tert-butyl ester 191 (0.100 g, 0.212 mmol) in methylene chloride (2 mL) was treated with trifluoroacetic acid (0.340 mL, 4.41 mmol). The reaction mixture was stirred at room temperature under an inert atmosphere for 6 hours. The volatiles were removed in vacuo with ethyl acetate to afford the trifluoroacetate salt of 2-[phenoxy-(2-piperazin-1-yl-ethyl)-phosphinoyloxy]-propionic acid ethyl ester 192 (0.103 g, 100%) (mixture of two diastereomers).

### Example 193

A solution of the phenol intermediate 45 (0.039 mmol) in dichloromethane (0.386 mL) was treated with triethylamine (0.022 mL, 0.155 mmol) and cat. 4-dimethylaminopyridine. The reaction mixture was cooled to 0° C. then triphosgene (0.023 g, 0.077 mmol) in a 1M solution of dichloromethane was added. The mixture stirred at room temperature under an inert atmosphere for 2 hours, then the free piperazine linker phosphonate 192 (0.056 g, 0.115 mmol) in a 1M solution of dichloromethane treated with triethylamine (0.022 mL, 0.155 mmol) was added, and the mixture was stirred overnight. The mixture was partitioned between dichloromethane and water. The organic phase was washed with saturated NH₄Cl and brine, dried (MgSO₄), and concentrated in vacuo. The residue was purified by silica gel chromatography (5/95—methanol/dichloromethane) to afford the product 193 (0.013 g, 50%) as a mixture of diastereomers: ¹H NMR (CDCl₃) δ 9.05 (dd, 1H), 8.1 (dd, 1H), 8.05 (s, 1H), 7.75 (d, 4H), 7.5 (dd, 1H), 7.4-7.1 (m, 11H), 7.05 (t, 2H), 5.1 (m, 1H), 4.8 (s, 2H), 4.2 (s, 2H), 4.15 (m, 2H), 3.8-3.4 (m, 4H), 3.0-2.2 (m, 8H), 1.6 & 1.4 (d, 3H), 1.2 (t, 3H); ³¹P NMR (CDCl₃) δ 28.30, 26.59; MS: 887 (M+1).

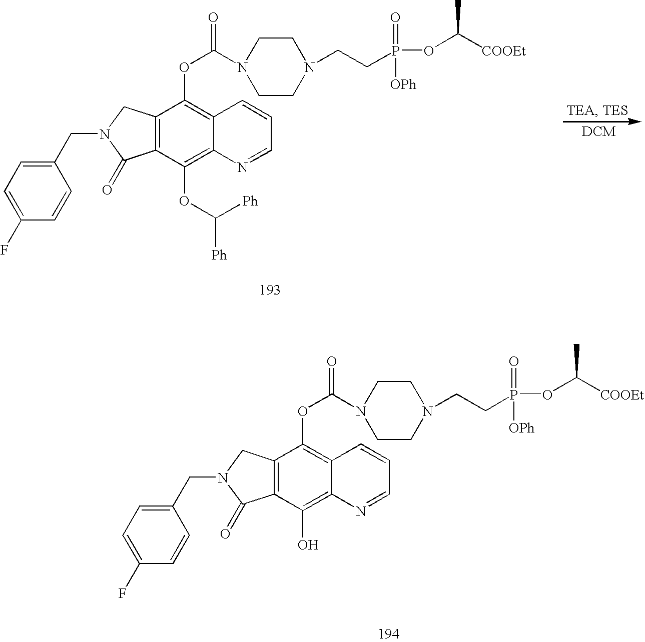

### Example 194

A solution of the phosphonate 193 (0.013 g, 0.015 mmol) in dichloromethane (0.5 mL) was treated with trifluoroacetic acid (0.1 mL) and triethylsilane (0.2 mL). The reaction mixture was stirred at room temperature under an inert atmosphere for 20 minutes. The volatiles were removed in vacuo with toluene. The solid was triturated in diethylether/hexane to afford the product 194 (0.010 g, 80%) as a TFA salt: ¹H NMR (CDCl₃) δ 8.95 (dd, 1H), 8.15 (dd, 1H), 7.55 (dd, 1H), 7.35 (m, 2H), 7.3-7.1 (m, 5H), 7.05 (t, 2H), 5.0 (m, 1H), 4.75 (s, 2H), 4.35 (s, 2H), 4.2 (m, 2H), 3.8-3.6 (m, 4H), 3.4-3.0 (m, 6H), 2.5-2.7 (m, 2H), 1.6 & 1.4 (d, 3H), 1.25 (t, 3H); ³¹P NMR (CDCl₃) δ 23.39, 21.67; MS: 721 (M+1).

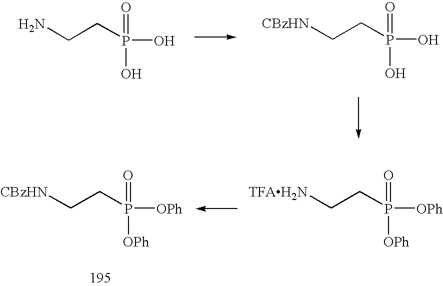

### Example 195

To a solution of 2-aminoethylphosphonic acid (1.26 g, 10.1 mmol) in 2N NaOH (10.1 mL, 20.2 mmol) was added benzyl chloroformate (1.7 mL, 12.1 mmol). After the reaction mixture was stirred for 2 d at room temperature, the mixture was partitioned between Et₂O and water. The aqueous phase was acidified with 6N HCl until pH=2. The resulting colorless solid was dissolved in MeOH (75 mL) and treated with Dowex 50WX8-200 (7 g). After the mixture was stirred for 30 minutes, it was filtered and evaporated under reduced pressure to give carbobenzoxyaminoethyl phosphonic acid (2.37 g, 91%) as a colorless solid.

To a solution of carbobenzoxyaminoethyl phosphonic acid (2.35 g, 9.1 mmol) in pyridine (40 mL) was added phenol (8.53 g, 90.6 mmol) and 1,3-dicyclohexylcarbodiimide (7.47 g, 36.2 mmol). After the reaction mixture was warmed to 70° C. and stirred for 5 h, the mixture was diluted with CH₃CN and filtered. The filtrate was concentrated under reduced pressure and diluted with EtOAc. The organic phase was washed with sat. NH₄Cl, sat. NaHCO₃, and brine, then dried over Na₂SO₄, filtered, and evaporated under reduced pressure. The crude product was chromatographed on silica gel twice (eluting 40-60% EtOAc/hexane) to give diphenyl 2-aminoethyl phosphonic acid (2.13 g, 57%) as a colorless solid.

To a solution of diphenyl 2-aminoethyl phosphonic acid (262 mg, 0.637 mmol) in iPrOH (5 mL) was added TFA (0.05 mL, 0.637 mmol) and 10% Pd/C (26 mg). After the reaction mixture was stirred under H₂ atmosphere (balloon) for 1 h, the mixture was filtered through Celite. The filtrate was evaporated under reduced pressure to give diphenyl carbobenzoxyaminoethyl phosphonate 195 (249 mg, 100%) as a colorless oil.

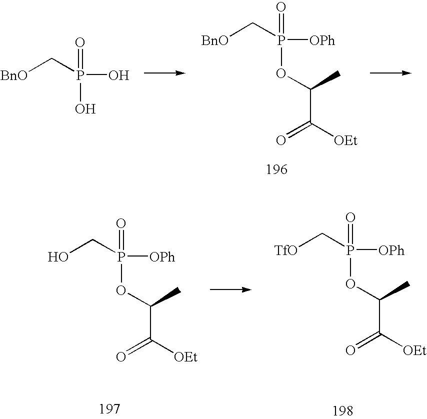

### Example 196

To a solution of benzyloxymethyl phosphonic acid (520 mg, 2.57 mmol) in CH₃CN (5 mL) was added thionyl chloride (0.75 mL, 10.3 mmol) and heated to 70° C. in an oil bath. After the reaction mixture was stirred for 2 h at 70° C., the mixture was concentrated and azeotroped with toluene. To a solution of the crude chloridate in toluene (5 mL) was added tetrazole (18 mg, 0.26 mmol) at 0° C. To this mixture was added phenol (121 mg, 1.28 mmol) and triethylamine (0.18 mL, 1.28 mmol) in toluene (3 mL) at 0° C. After the reaction mixture was warmed to room temperature and stirred for 2 h, ethyl lactate (0.29 mL, 2.57 mmol) and triethylamine (0.36 mL, 2.57 mmol) in toluene (2.5 mL) were added. The reaction mixture was stirred for 16 hours at room temperature, at which time the mixture was partitioned between EtOAc and sat. NH₄Cl. The organic phase was washed with sat. NH₄Cl, 1M NaHCO₃, and brine, then dried over Na₂SO₄, filtered, and evaporated under reduced pressure. The crude product was chromatographed on silica gel (eluting 20-40% EtOAc/hexane) to give two diastereomers (isomer A and isomer B) of 2-(benzyloxymethyl-phenoxy-phosphinoyloxy)-propionic acid ethyl ester 196 (66 mg, 109 mg, 18% total) as colorless oils.

### Example 197a

To a solution of benzyl phosphonate 196 isomer A (66 mg, 0.174 mmol) in EtOH (2 mL) was added 10% Pd/C (13 mg). After the reaction mixture was stirred under H₂ atmosphere (balloon) for 6 h, the mixture was filtered through Celite. The filtrate was evaporated under reduced pressure to give alcohol 197a isomer A (49 mg, 98%) as a colorless oil.

### Example 197b

To a solution of benzyl phosphonate 196 isomer B (110 mg, 0.291 mmol) in EtOH (3 mL) was added 10% Pd/C (22 mg). After the reaction mixture was stirred under H₂ atmosphere (balloon) for 6 h, it was filtered through Celite. The filtrate was evaporated under reduced pressure to give alcohol 197b isomer B (80 mg, 95%) as a colorless oil.

### Example 198a

To a solution of alcohol 197a isomer A (48 mg, 0.167 mmol) in CH₂Cl₂ (2 mL) was added 2,6-lutidine (0.03 mL, 0.250 mmol) and trifluoromethanesulfonic anhydride (0.04 mL, 0.217 mmol) at −40° C. (dry ice-CH₃CN bath). After the reaction mixture was stirred for 15 min at −40° C., the mixture was warmed to 0° C. and partitioned between Et₂O and 1M H₃PO₄. The organic phase was washed with 1M H₃PO₄ (3 times), dried over Na₂SO₄, filtered, and evaporated under reduced pressure to give triflate 198a isomer A (70 mg, 100%) as a pale yellow oil.

### Example 198b

To a solution of alcohol 197b isomer B (80 mg, 0.278 mmol) in CH₂Cl₂ (3 mL) was added 2,6-lutidine (0.05 mL, 0.417 mmol) and trifluoromethanesulfonic anhydride (0.06 mL, 0.361 mmol) at −40° C. (dry ice-CH₃CN bath). After the reaction mixture was stirred for 15 min at −40° C., the mixture was warmed to 0° C. and partitioned between Et₂O and 1M H₃PO₄. The organic phase was washed with 1M H₃PO₄ (3 times), dried over Na₂SO₄, filtered, and evaporated under reduced pressure to give triflate 198b isomer B (115 mg, 98%) as a pale yellow oil.

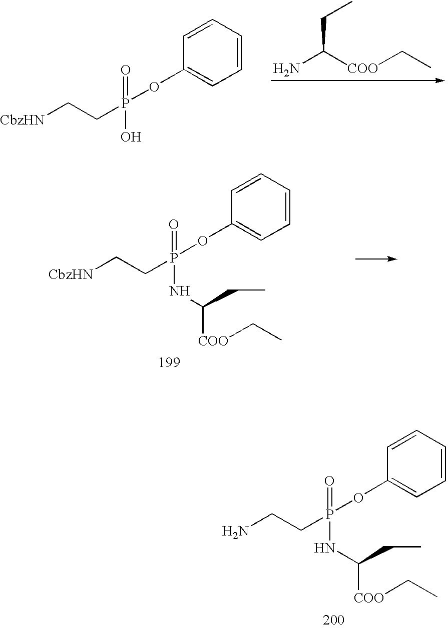

### Example 199

To a stirred solution of phenyl 2-carbobenzoxyaminoethyl phosphonate (1 g, 3 mmol) in 30 mL of acetonitrile at room temperature under N₂ was added thionyl chloride (0.67 mL, 9 mmol). The resulted mixture was stirred at 60-70° C. for 0.5 h. After cooled to room temperature, the solvent was removed under reduced pressure, and the residue was added 30 mL of DCM, followed by DIEA (1.7 mL, 10 mmol), L-alanine butyric acid ethyl ester hydrochloride (1.7 g, 10 mmol) and TEA (1.7 mL, 12 mmol). After 4 h at room temperature, the solvent was removed under reduced pressure, and the residue was diluted with DCM and washed with brine and water, dried over Na₂SO₄, filtered and concentrated. The residue was purified by chromatography on silica gel (Hexane/EtOAc 1:1) to give 199 (670 mg, 50%) as a yellow oil. ¹H NMR (CDCl₃) δ 7.33-7.11 (m, 10H), 5.70 (m, 1H), 5.10 (s, 2H), 4.13-3.53 (m, 5H), 2.20-2.10 (m, 2H), 1.76-1.55 (m, 2H), 1.25-1.19 (m, 3H), 0.85-0.71 (m, 3H); ³¹P NMR (CDCl₃) δ 30.2 and 29.9; MS (ESI) 471 (M+Na).

### Example 200

A solution of compound 199 (450 mg) was dissolved in 9 mL of EtOH, then 0.15 mL of acetic acid and 10% Pd/C (90 mg) was added. The resulted mixture was stirred under H2 atmosphere (balloon) for 4 h. After filtration through Celite, the filtered was evaporated under reduced pressure to afford the compound 200 (300 mg, 95%) as a colorless oil. ¹H NMR (CDCl₃) δ 7.29-7.12 (m, 5H), 4.13-3.53 (m, 5H), 2.20-2.10 (m, 2H), 1.70-1.55 (m, 2H), 1.24-1.19 (m, 3H), 0.84-0.73 (m, 3H); ³¹P NMR (CDCl₃) δ 29.1 and 28.5; MS (ESI) 315 (M+1).

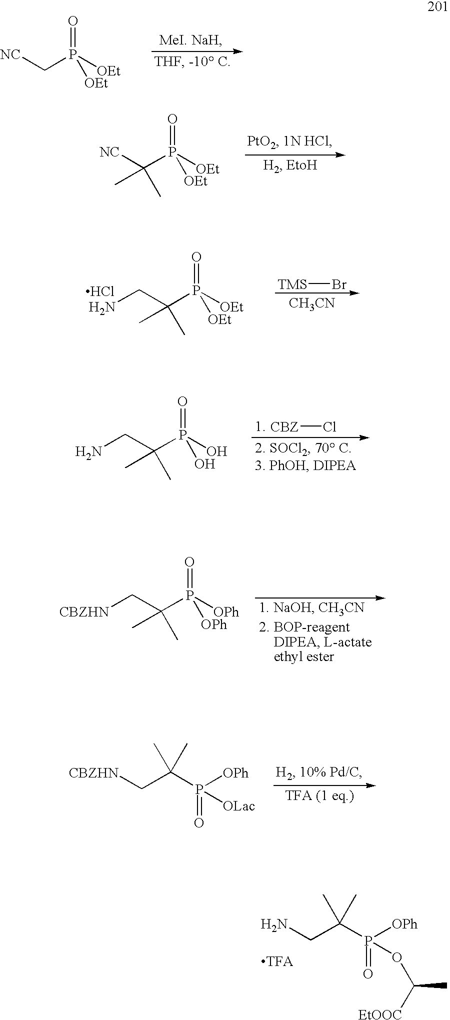

### Example 201

A THF solution (30 mL) of NaH (3.4 g of 60% oil dispersion, 85 mmol) was cooled to −10° C., followed by the addition of diethyl (cyanomethyl)phosphonate (5 g, 28.2 mmol) and iodomethane (17 g, 112 mmol). The resulting solution was stirred at −10° C. for 2 hr, then 0° C. for 1 hr, was worked up, and purified to give diethyl (cyano(dimethyl)methyl) phosphonate (5 g, 86%).

Diethyl (cyano(dimethyl)methyl) phosphonate was reduced to the amine derivative by the described procedure (J. Med. Chem. 1999, 42, 5010-5019) whereby a solution of ethanol (150 mL) and 1N HCl aqueous solution (22 mL) of diethyl (cyano(dimethyl)methyl) phosphonate (2.2 g, 10.7 mmol) was hydrogenated at 1 atmosphere in the presence of PtO₂ (1.25 g) at room temperature overnight. The catalyst was filtered through a Celite pad. The filtrate was concentrated to dryness, to give crude diethyl 2-amino-1,1-dimethyl-ethyl phosphonate (2.5 g, as HCl salt).

Crude diethyl 2-amino-1,1-dimethyl-ethyl phosphonate (2.5 g) in 30 mL CH₃CN was cooled to 0° C., and treated with TMSBr (8 g, 52 mmol) for 5 hr. The reaction mixture was stirred with methanol for 1.5 hr at room temperature, concentrated, recharged with methanol, concentrated to dryness to give crude 2-Amino-1,1-dimethyl-ethyl phosphonic acid which was used for next reaction without further purification.

2-Amino-1,1-dimethyl-ethyl phosphonic acid was protected with CBZ, followed by the reaction with thionyl chloride at 70° C. The CBZ protected dichloridate was reacted with phenol in the presence of DIPEA. Removal of one phenol, follow by coupling with ethyl L-lactate gave N-CBZ-2-amino-1,1-dimethyl-ethyl phosphonate derivative. Hydrogenation of N-CBZ derivative at 1 atmosphere in the presence of 10% Pd/C and 1 eq. of TFA gave lactate phenyl (2-amino-1,1-dimethyl-ethyl)phosphonate 201 as the TFA salt.

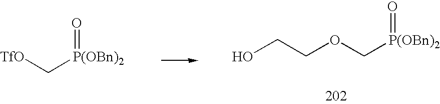

### Example 202

Powdered magnesium tert-butoxide (2.05 g, 12.02 mmol) was added to a solution of dibenzyl trifluoromethane sulfonic hydroxymethyl phosphonate (4.10 g, 9.66 mmol) and anhydrous ethylene glycol (5.39 mL, 96.6 mmol) in anhydrous DMF (30 mL) at 0° C. The reaction mixture was stirred at 0° C. for 1.5 h, then concentrated. The residue was partitioned between EtOAc and H₂O and washed with 1 N HCl, saturated NaHCO₃ solution, and brine. Organic layer dried (MgSO₄), concentrated and purified (silica gel, 4% MeOH/CH₂Cl₂) to give (2-hydroxy-ethoxymethyl)-phosphonic acid dibenzyl ester 202 as a colorless oil (1.55 g, 48%). ¹H NMR (300 MHz, CDCl₃): δ 7.37 (s, 10H, Ar), 5.40-5.05 (m, 4H, CH₂Ph), 3.84 (d, J=8.1 Hz, 2H, PCH₂O), 3.70-3.60 (m, 4H, OCH₂CH₂O, OCH₂CH₂O); ³¹P NMR (121 MHz, CDCl₃): δ 22.7.

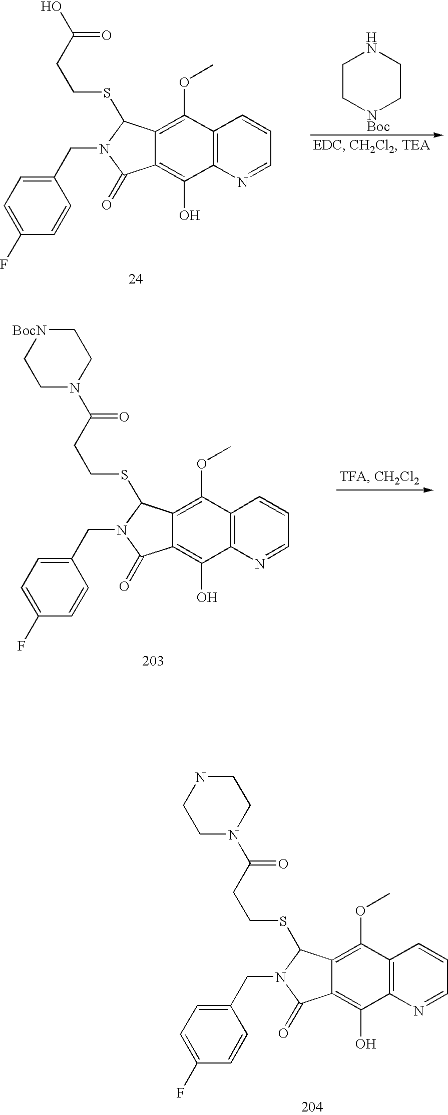

### Example 203

A solution of 24 (Example 24) (38 mg, 0.086 mmol) in CH₂Cl₂ (0.86 mL) was stirred with EDC (33 mg, 0.172 mmol), TEA (12 μL, 0.086 mmol), and 1-Boc-piperazine (19 mg, 0.103 mmol) at ambient temperature for 15 h when LCMS analysis demonstrated completion of the reaction. The reaction mixture was worked up by dilution of the mixture with CH₂Cl₂ and washing the organic layer with H₂O. The organic layer was dried in vacuo and the residue, 4-{3-[7-(4-fluoro-benzyl)-9-hydroxy-5-methoxy-8-oxo-7,8-dihydro-6H-pyrrolo[3,4-g]quinolin-6-ylsulfanyl]-propionyl}-piperazine-1-carboxylic acid tert-butyl ester 203 was carried forward for deprotection.

### Example 204

A solution of 203 (52 mg, 0.085 mmol) in 0.8 mL of trifluoroacetic acid and 0.8 mL of CH₂Cl₂ was stirred at room temperature for 1 h when the starting material was completely consumed as detected by LCMS. The solution was dried in vacuo and re-dissolved in 1:1 mixture of MeOH—H₂O. The product 204 was purified by RP-HPLC using a 5-95% A. Buffer A contained CH₃CN-1% TFA and buffer B was H₂O-1% TFA. ¹H NMR (300 MHz, CD₃OD) δ 2.19-2.40 (m, 4H), 3.06-3.20 (m, 4H), 3.43-3.56 (m, 2H), 3.63-3.74 (m, 2H), 4.08 (s, 3H), 4.62 (d, 1H, J=15 Hz), 5.16 (d, 1H, J=15 Hz), 5.76 (s, 1H), 7.10 (t, 2H, J=9 Hz), 7.46 (t, 2H, J=8 Hz), 7.74 (dd, 1H, J=4, 8 Hz), 8.69 (d, 1H, J=8 Hz), 8.96 (d, 1H, J=4 Hz); ¹⁹F NMR (282.6 MHz, CD₃OD) δ −77.7, 60.0; EI MS (m/z) 511.0 [M+H]⁺.

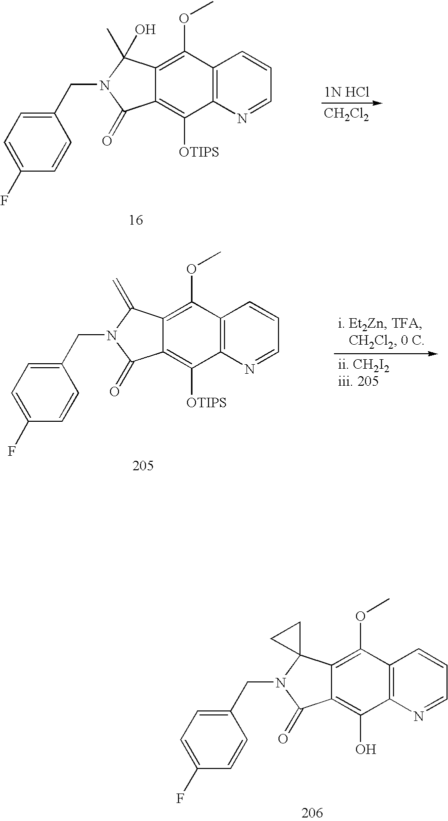

### Example 205

Grignard product 16 (Example 16) was worked up by addition of ethyl acetate and stirring of the organic layer with aqueous 1N HCl for 30 minutes. The layers were separated and the organic layer was washed with the 1N HCl solution 2 more times. The organic layer was checked with LCMS to assure complete elimination of the alcohol resulted from the Grignard reaction to the eliminated product 205. The organic layer was dried in vacuo and the residue was purified by column chromatography using CH₂Cl₂ to give 205. ¹H NMR (300 MHz, CDCl₃) δ 1.15 (d, 18H, J=8 Hz), 1.56 (septet, 3H, J=8 Hz), 3.95 (s, 3H), 4.82 (s, 1H), 4.99 (s, 2H), 5.53 (s, 1H), 7.01 (t, 2H, J=8 Hz), 7.28 (dd, 2H, J=5, 9 Hz), 7.54 (dd, 1H, J=4, 8 Hz), 8.46 (d, 1H, J=8 Hz), 8.87 (d, 1H, J=3 Hz); ¹⁹F NMR (282.6 MHz, CDCl₃) δ 61.06; EI MS (m/z) 507.4 [M+H]⁺.

### Example 206

A solution of diethylzinc (0.134 mmol, 134 μL of a 1M mixture) and 134 μL of CH₂Cl₂ was added to TFA (0.134 mmol, 10.4 μL) under a N₂ atmosphere at 0° C. The mixture was stirred at cooled temperature for 15 minutes, then a solution of CH₂I₂ (0.134 mmol, 11 μL) in 100 μL of CH₂Cl₂ was added. After 10 minutes, a solution of 205 in 100 μL of CH₂Cl₂ was added and the ice bath removed. The reaction mixture was stirred at ambient temperature for 1 hour when LCMS analysis demonstrated complete consumption of the starting materials. The product 206 was purified by RP-HPLC using a 20-80% A. Buffer A contained CH₃CN-1% TFA and buffer B was H₂O-1% TFA. ¹H NMR (300 MHz, CD₃OD) δ 1.58 (t, 2H, J=5 Hz), 1.79 (t, 2H, J=5 Hz), 3.95 (s, 3H), 4.61 (s, 2H), 7.07 (t, 2H, J=9 Hz), 7.32 (dd, 2H, J=5, 8 Hz), 7.84 (dd, 1H, J=4, 8 Hz), 8.77 (d, 1H, J=8 Hz), 8.98 (d, 1H, J=4 Hz); ¹⁹F NMR (282.6 MHz, CD₃OD) δ −78.0, 59.3; EI MS (m/z) 365.3 [M+H]⁺, 387.3 [M+Na]⁺.

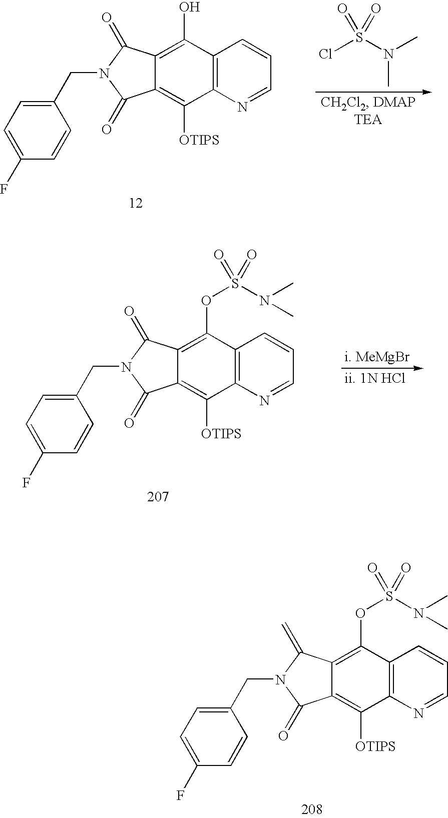

### Example 207

A solution of 12 (Example 12, 65 mg, 0.131 mmol) in 1.3 mL of CH₂Cl₂ was stirred with dimethyl sulfamoyl chloride (38 mg, 0.262 mmol), TEA (73 μL, 0.63 mmol), and DMAP (2 mg, 0.013 mmol) for 2 hours at room temperature when LCMS analysis demonstrated complete consumption of the starting materials. The reaction was worked up by dilution with CH₂Cl₂ and washing the organic layer with H₂O. The solvent was removed under reduced pressure and the product was purified by column chromatography to yield 59 mg of 207 (75%) as a white solid. ¹H NMR (300 MHz, CDCl₃) δ 1.12 (d, 18H, J=8 Hz), 1.53 (septet, 3H, J=8 Hz), 3.23 (s, 6H), 4.84 (s, 2H), 7.00 (t, 2H, J=8 Hz), 7.45 (dd, 2H, J=6, 9 Hz), 7.65 (dd, 1H, J=4, 8 Hz), 8.77 (dd, 1H, J=2, 8 Hz), 8.94 (dd, 1H, J=2, 4 Hz); ¹⁹F NMR (282.6 MHz, CDCl₃) δ 62.0; EI MS (m/z) 624.2 [M+Na]⁺.

### Example 208

A solution of 207 (30 mg, 0.050 mmol) in 0.25 mL of THF was stirred with 33 μL (0.10 mmol) of methylmagnesium bromide for 1 hour at room temperature. The solution was diluted with CH₂Cl₂ and stirred with aqueous 1N HCl for 30 minutes. Removal of the solvent in vacuo yielded 26 mg (87%) of the product 208 as a green oil. ¹H NMR (300 MHz, CDCl₃) δ 1.14 (d, 18H, J=8 Hz), 1.56 (septet, 3H, J=8 Hz), 2.97 (s, 6H), 4.94 (s, 1H), 5.00 (s, 2H), 5.59 (s, 1H), 7.00 (t, 2H, J=8 Hz), 7.21-7.32 (m, 2H), 7.55-7.62 (m, 1H), 8.50 (d, 1H, J=8 Hz), 8.88 (br s, 1H); ¹⁹F NMR (282.6 MHz, CDCl₃) δ 61.3; EI MS (m/z) 600.2 [M+H]⁺, 622.2 [M+Na]⁺.

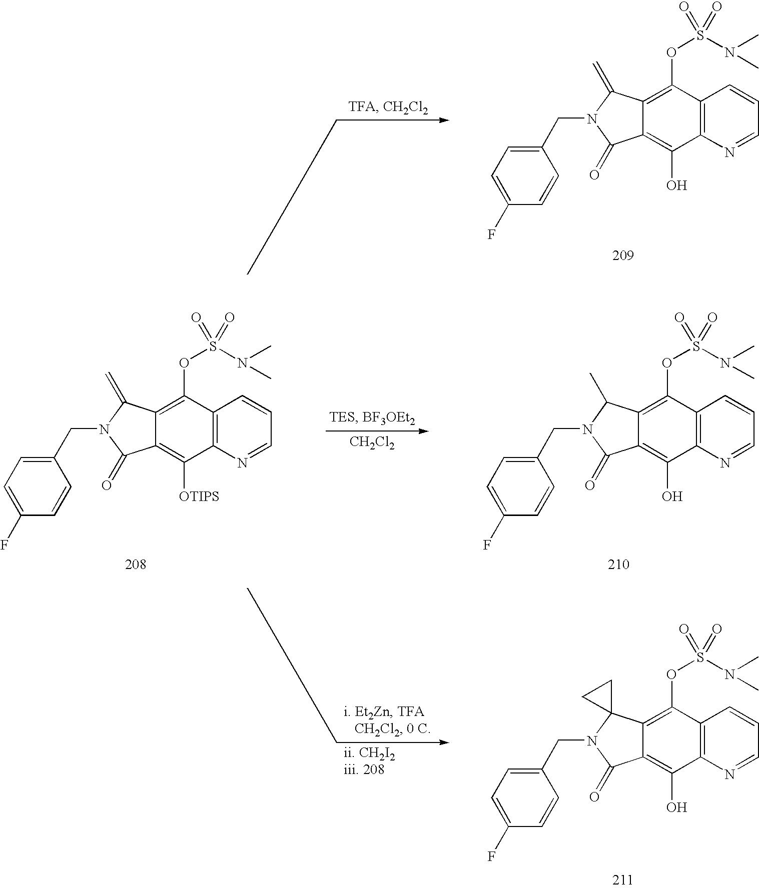

### Example 209

A solution of 208 (13 mg, 0.022 mmol) and TFA (0.11 mL) and CH₂Cl₂ (0.11 mL) was allowed to stir at room temperature overnight. The solvent was removed in vacuo and the residue was purified by RP-HPLC using a 20-80% A to give product 209. Buffer A contained CH₃CN-1% TFA and buffer B was H₂O-1% TFA. ¹H NMR (300 MHz, CDCl₃) δ 3.06 (s, 3H), 3.07 (s, 3H), 5.00 (s, 2H), 5.12 (s, 1H), 5.71 (s, 1H), 6.96-7.07 (m, 2H), 7.22-7.33 (m, 2H), 7.71 (dd, 1H, J=4, 9 Hz), 8.67 (d, 1H, J=8 Hz), 9.05 (br s, 1H); ¹⁹F NMR (282.6 MHz, CDCl₃) δ −76.2, 62.1; EI MS (m/z) 444.2 [M+H]⁺, 466.1 [M+Na]⁺.

### Example 210

Under a N₂ atmosphere, a solution of 208 (14 mg, 0.023 mmol) in CH₂Cl₂ (0.23 mL) was stirred with triethylsilane (15 μL, 0.093 mmol) and boron trifluoride diethyletherate (BF₃OEt₂, 20 μL, 0.164 mmol) at ambient temperature overnight. The reaction mixture was worked up by removing the solvent under reduced pressure and precipitation from EtOAc-Hex to provide 7.5 mg of the product 210 as a yellow solid. ¹H NMR (300 MHz, CDCl₃) δ 1.56 (d, 3H, J=7 Hz), 3.16 (s, 6H), 4.42 (d, 1H, J=15 Hz), 5.02 (q, 1H, J=6 Hz), 5.09 (d, 1H, J=15 Hz), 7.06 (t, 2H, J=8 Hz), 7.33 (dd, 2H, J=5, 9 Hz), 7.72-7.79 (m, 1H), 8.62 (d, 1H, J=9 Hz), 9.15 (br s, 1H); ¹⁹F NMR (282.6 MHz, CDCl₃) δ −76.2, 62.5; EI MS (m/z) 446.2 [M+H]⁺, 468.2 [M+Na]⁺.

### Example 211

Under a N₂ atmosphere, to a solution of diethylzinc (0.074 mmol, 74 μL of a 1M mixture) and 74 μL of CH₂Cl₂ was added TFA (0.074 mmol, 5.7 μL) at 0° C. This mixture was stirred at cooled temperature for 15 minutes when a solution of CH₂I₂ (0.074 mmol, 6 μL) in 50 μL of CH₂Cl₂ was added. After 10 minutes, a solution of 208 in 50 μL of CH₂Cl₂ was added and the ice bath removed. The reaction mixture was stirred at ambient temperature for 1 hour when LCMS analysis demonstrated complete consumption of the starting materials. The product 211 was purified by RP-HPLC using a 20-80% A. Buffer A contained CH₃CN-1% TFA and buffer B was H₂O-1% TFA. ¹H NMR (300 MHz, CD₃OD) δ 1.46 (br t, 2H), 2.10 (br t, 2H), 3.14 (s, 6H), 4.55 (s, 2H), 7.02 (t, 2H, J=9 Hz), 7.21-7.31 (m, 2H), 7.60-7.68 (m, 1H), 8.58-8.65 (m, 1H), 9.05-9.08 (m, 1H); EI MS (m/z) 458.2 [M+H]⁺, 480.1 [M+Na]⁺.

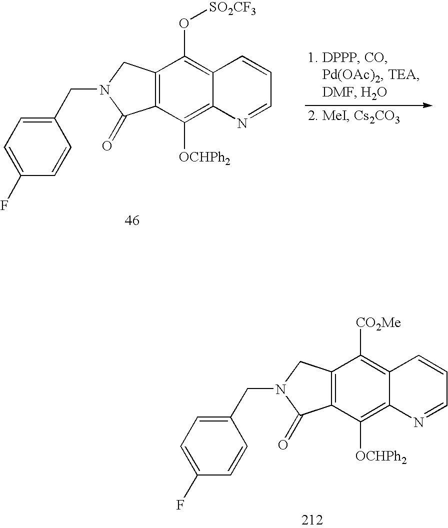

### Example 212

To trifluoro-methanesulfonic acid 9-benzhydryloxy-7-(4-fluoro-benzyl)-8-oxo-7,8-dihydro-6H-pyrrolo[3,4-g]quinolin-5-yl ester 46 (1.48 g, 2.39 mmol) and 1,3-bis(diphenylphosphino)propane (DPPP) (295 mg, 0.7 mmol) in DMF (20 mL) and water (1 mL) in a two-necked round bottom flask were added Pd(OAc)₂ (107 mg, 0.48 mmol). The solution was degassed under high vacuum and flushed with carbon monoxide from a balloon. The flushing was repeated five times. TEA (0.733 mL, 3.26 mmol) was introduced. The mixture was heated under CO atmosphere for 2.5 hours and cooled down to the room temperature. MeI (0.74 mL, 12 mmol) and Cs₂CO₃ were added and stirring was continued under a nitrogen atmosphere for 45 minutes. The mixture was diluted with EtOAc (300 mL), washed with water, 1N aqueous HCl and brine, dried over MgSO₄ and concentrated. The crude product was purified by chromatography on a silica gel column eluting with 15% to 35% of EtOAc in hexane to afford 9-benzhydryloxy-7-(4-fluoro-benzyl)-8-oxo-7,8-dihydro-6H-pyrrolo[3,4-g]quinoline-5-carboxylic acid methyl ester 212, (0.9 g, 1.69 mmol, 70%) as a yellow solid. ¹H NMR (CDCl₃): δ9.25 (d, 1H), 9.05 (m, 1H), 7.80 (d, 4H), 7.56 (dd, 1H), 7.0-7.4 (m, 11H), 4.85 (s, 2H), 4.55 (s, 2H), 3.95 (s, 3H); MS: 555 (M+Na).

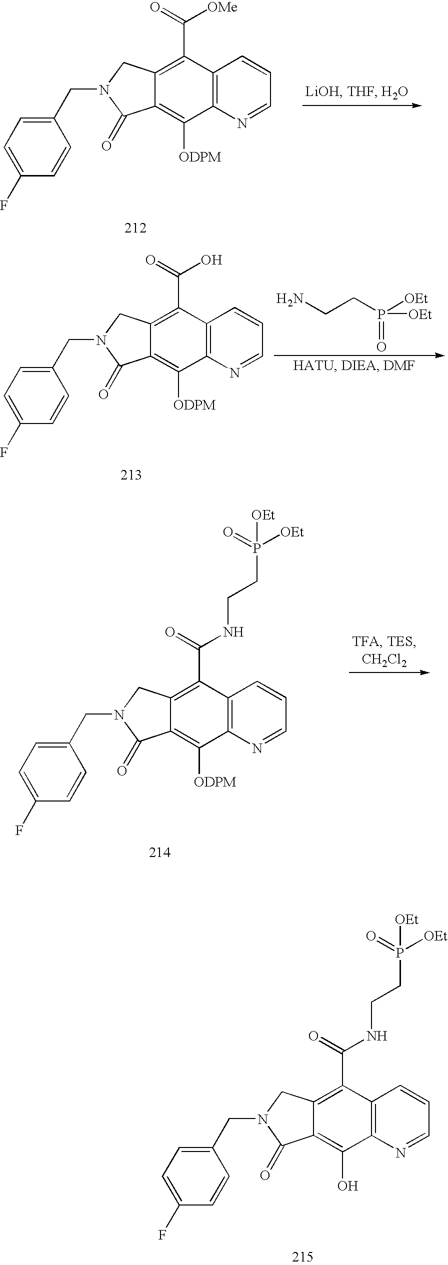

### Example 213

A solution of 9-benzhydryloxy-7-(4-fluoro-benzyl)-8-oxo-7,8-dihydro-6H-pyrrolo[3,4-g]quinoline-5-carboxylic acid methyl ester 212 (54 mg, 0.10 mmol) in 1.0 mL of a 1:1:1 mixture of THF:MeOH:H₂O was stirred with LiOH (9.7 mg, 0.41 mmol) overnight when the starting materials were completely consumed as judged by TLC (DPM=benzhydryl, Ph₂CH—). The reaction mixture was dried under reduced pressure and the residue was dissolved in EtOAc. The organic layer was stirred with saturated aqueous NH₄Cl for 30 minutes. The aqueous layer was checked by TLC to assure complete transfer of the products to the organic layer. The organic layer was dried in vacuo to yield 45.5 mg (87%) of 9-benzhydryloxy-7-(4-fluoro-benzyl)-8-oxo-7,8-dihydro-6H-pyrrolo[3,4-g]quinoline-5-carboxylic acid 213 as a white solid. The product was carried on without purification. MS (m/z) 519.2 [M+H]⁺, 541.2 [M+Na]⁺.

Alternatively, methyl ester 212 (0.071 g, 0.1334 mmol) was dissolved in 2.4 mL of tetrahydrofuran and 0.6 mL of DI H₂O. To this was added LiOH (0.013 g, 0.5338 mmol) and mixture stirred at room temperature. After 15 hours, starting material consumed. Diluted with dichloromethane, washed with 1M HCl solution, dried (Na₂SO₄), concentrated to give 213 (0.068 g, 0.1313 mmol, 98%.) ¹H NMR (CD₃SOCD₃) δ 9.25 (d, 1H), 9.12 (dd, 1H), 8.17 (s, 1H), 7.75 (d, 5H), 7.37 (dd, 2H), 7.24 (m, 6H), 4.82 (s, 2H), 4.59 (s, 2H.) MS: 517 (M−1.)

### Example 214

A solution of the oxalate salt (HO₂CCO₂⁻) of diethyl(aminoethyl)phosphonate (12 mg, 0.042 mmol) in 0.21 mL of DMF was mixed with DIEA (15 μL, 0.084 mmol) until the reaction became clear. To this solution was added 213 (11 mg, 0.021 mmol) and O-(7-azabenzotriazol-1-yl)-N,N,N′,N′-tetramethyluronium hexafluorophosphate (HATU) (16 mg, 0.042 mmol). This mixture was stirred at room temperature for 2 hours when it was warmed to 60° C. with a heat gun for 1 minute. LCMS analysis demonstrated complete consumption of the starting materials. The reaction mixture was directly loaded onto a silica gel column and the product was quickly eluted with a gradient of EtOAc-10% MeOH/EtOAc to provide 12.7 mg (88%) of the product 214. ¹H NMR (300 MHz, CD₃OD) δ 1.29 (t, 6, J=7 Hz), 2.18 (dt, 2H, J=7, 18 Hz), 3.53-3.65 (m, 2H), 4.08 (septet, 4H, J=7 Hz), 4.46 (s, 2H), 4.83 (s, 2H), 7.06-7.25 (m, 8H), 7.40 (dd, 2H, J=5, 9 Hz), 7.61-7.68 (m, 6H), 8.04 (s, 1H), 8.44 (d, 1H, J=7 Hz), 9.04-9.09 (m, 1H); ³¹P (121.4 MHz, CD₃OD) δ 29.5; MS (m/z) 682.1 [M+H]⁺, 704.2 [M+Na]⁺.

### Example 215

A solution of 214 (12.7 mg, 0.019 mmol) in 0.19 mL of CH₂Cl₂ was stirred with TFA (144 μL, 1.9 mmol) and TES (304 μL, 1.9 mmol) for 45 minutes under a N₂ atmosphere. TLC and LCMS analysis indicated complete reaction at that time. The reaction was worked up by removing the solvent under reduced pressure. The residue was purified by crystallization from EtOAc-Hex to yield 8.6 mg (71%) of (2-{[7-(4-fluoro-benzyl)-9-hydroxy-8-oxo-7,8-dihydro-6H-pyrrolo[3,4-g]quinoline-5-carbonyl]-amino}-ethyl)-phosphonic acid diethyl ester 215 as a yellow solid. ¹H NMR (500 MHz, CD₃OD) δ 1.33 (t, 6H, J=7 Hz), 2.24 (dt, 2H, J=19, 7 Hz), 3.70 (septet, 2H, J=8 Hz), 4.09-4.17 (m, 4H), 4.61 (s, 2H), 4.78 (s, 2H), 7.10 (t, 2H, J=9 Hz), 7.41 (dd, 2H, J=6, 8 Hz), 7.76 (br d, 1H, J=5 Hz), 8.71 (d, 1H, J=9 Hz), 8.95 (br s, 1H); ³¹P (121.4 MHz, CD₃OD) δ 29.5; MS (m/z) 516.3 [M+H]⁺, 1030.9 [2M]+, 1053.0 [2M+Na]⁺.

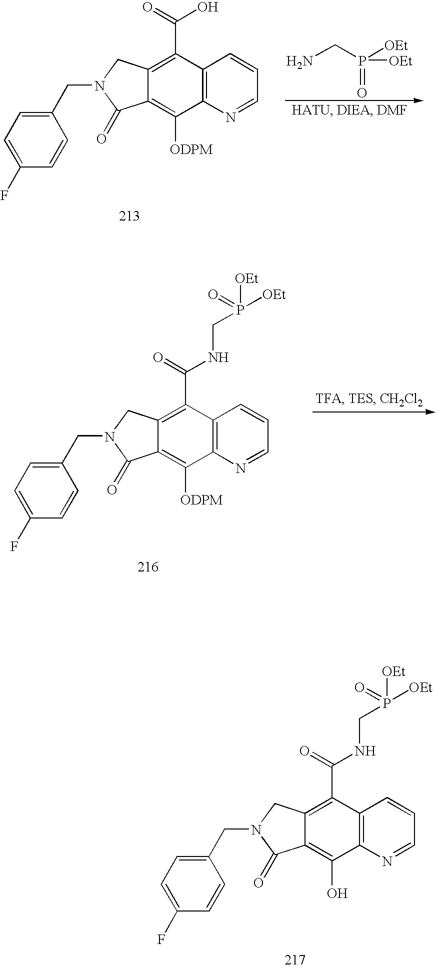

### Example 216

A solution of oxalate salt of diethyl(aminomethyl)phosphonate (8 mg, 0.031 mmol) in 0.31 mL of DMF and DIEA (22 μL, 0.124 mmol) was added to 213 (16 mg, 0.031 mmol) and HATU (24 mg, 0.062 mmol). The solution was stirred at ambient temperature for 2 hours when another batch of the amine and the coupling reagent equivalent to the above amounts were added. The reaction was heated with a heat gun to 60° C. for 1 minute and the reaction was analyzed by LCMS. The reaction mixture was loaded onto a flash column and ({[9-benzhydryloxy-7-(4-fluoro-benzyl)-8-oxo-7,8-dihydro-6H-pyrrolo[3,4-g]quinoline-5-carbonyl]-amino}-methyl)-phosphonic acid diethyl ester 216 was eluted with EtOAc-10% MeOH to provide 20 mg (97%) of a clear oil. MS (m/z) 668.1 [M+H]⁺, 690.3 [M+Na]⁺.

### Example 217

A solution of 216 (20 mg, 0.030 mmol) in 0.30 mL of CH₂Cl₂ was stirred with TFA (231 μL, 3.00 mmol) and TES (479 μL, 3.00 mmol) for 30 minutes when the starting materials were completely consumed as judged by TLC and LCMS. The reaction was worked up by removal of the solvent in vacuo and crystallizing the product from EtOAc-Hex to provide 10 mg (66%) of ({[7-(4-Fluoro-benzyl)-9-hydroxy-8-oxo-7,8-dihydro-6H-pyrrolo[3,4-g]quinoline-5-carbonyl]-amino}-methyl)-phosphonic acid diethyl ester 217 as a yellow solid. ¹H NMR (300 MHz, CD₃OD) δ 1.32 (t, 6H, J=7 Hz), 3.96 (d, 2H, J=12 Hz), 4.16 (septet, 4H, J=7 Hz), 4.56 (s, 2H), 4.79 (s, 2H), 7.10 (t, 2H, J=9 Hz), 7.39 (dd, 2H, J=9 Hz), 7.76 (br s, 1H), 8.66 (d, 1H, J=8 Hz), 8.95 (br s, 1H); ³¹P (121.4 MHz, CD₃OD) δ 23.2; ¹⁹F NMR (282.6 MHz, CD₃OD) δ −76.2, 59.9; MS (m/z) 502.5 [M+H]⁺, 1003.0 [2M]+, 1025.1 [2M+Na]⁺.

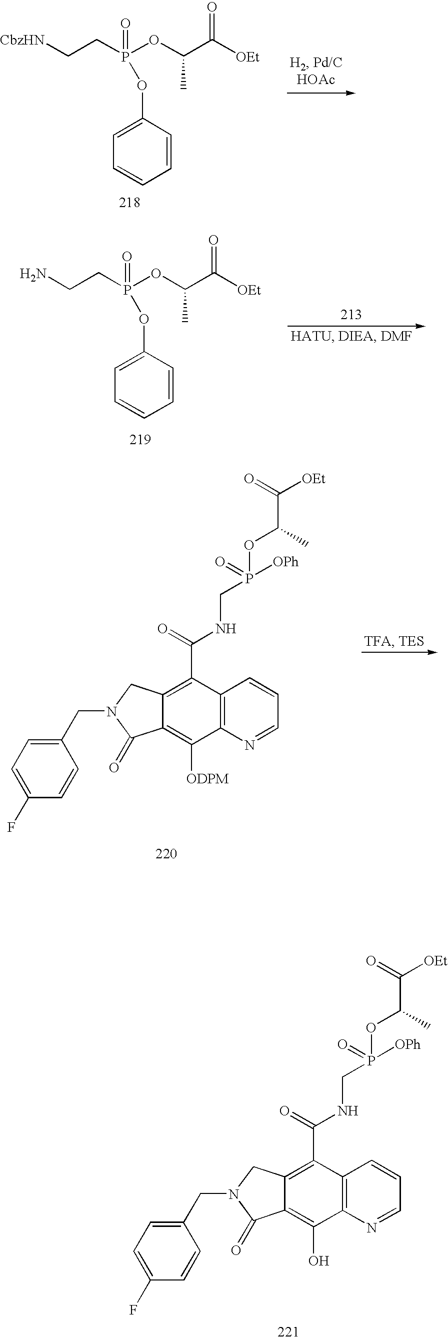

### Example 218

S-lactate ester 218 was prepared from phenyl 2-carbobenzoxyaminoethyl phosphonate by the coupling and hydrogenation procedures described in Example 201.

### Example 219

A solution of 2-[(2-benzyloxycarbonylamino-ethyl)-phenoxy-phosphinoyloxy]-propionic acid ethyl ester 218 (240 mg, 0.551 mmol) with approximately 50% purity and a ratio of 2:1 of diastereomers was dissolved in 5.5 mL of ethanol with acetic acid (63 μL, 1.10 mmol). To this solution was added 36 mg of 10% Pd/C and the solution was degassed under a hydrogen atmosphere three times. The solution was vigorously stirred at room temperature for 3 hours when TLC showed complete consumption of the starting materials. The mixture was filtered through a pad of Celite and dried to provide 174 mg (87%) of 2-[(2-amino-ethyl)-phenoxy-phosphinoyloxy]-propionic acid ethyl ester; compound with acetic acid 219 as a clear oil.

### Example 220

A solution of 13.5 mg of 213 in 0.13 mL of DMF was stirred with HATU (20 mg, 0.052 mmol) at room temperature for 10 minutes. To this solution was added a premixed solution of 219 (28 mg, 0.078 mmol) of approximately 50% purity in 0.130 mL of DMF and DIEA (13.4 mg, 0.104 mmol). The reaction mixture was gently heated with a heat gun for 30 seconds and then the reaction was allowed to proceed at room temperature for 2 hours when LCMS demonstrated complete consumption of the carboxylic acid. The reaction mixture was loaded onto a silica gel column and purified with EtOAc-10% MeOH to provide 9.5 mg of 3-[(2-{[9-Benzhydryloxy-7-(4-fluoro-benzyl)-8-oxo-7,8-dihydro-6H-pyrrolo[3,4-g]quinoline-5-carbonyl]-amino}-ethyl)-phenoxy-phosphinoyl]-2-methyl-propionic acid ethyl ester 220 which was carried on to the next step.

### Example 221

A solution of 220 (9.5 mg, 11.8 μmol) was stirred with 0.12 mL of dry dichloromethane with trifluoroacetic acid (93 μL, 1.18 mmol) and triethylsilane (189 μL, 1.18 mmol) for 1 hour at room temperature when TLC showed complete consumption of the starting materials. The reaction mixture was dried in vacuo and azeotroped from dichloromethane three times. The solid product was triturated with EtOAc-Hex to get 6 mg of 2-[(2-{[7-(4-Fluoro-benzyl)-9-hydroxy-8-oxo-7,8-dihydro-6H-pyrrolo[3,4-g]quinoline-5-carbonyl]-amino}-ethyl)-phenoxy-phosphinoyloxy]-propionic acid ethyl ester 221 as a pale yellow solid. The NMR of the two diastereomers in CDCl₃ is broad and indicates presence of rotamers. VT NMR in DMSO at 85° C. resulted in drastic sharpening of the peaks. ¹H NMR (300 MHz, DMSO-d6, 85° C.) δ 1.15-1.26 (m, 3H), 1.35 and 1.47 (d, 3H, J=7 Hz), 2.23-2.45 (m, 2H), 3.58-3.57 (m, 2H), 4.08-4.19 (m, 2H), 4.56 (s, 2H), 4.69 (s, 2H), 4.93-5.04 (m, 1H), 7.14 (t, 2H, J=9 Hz), 7.18-7.23 (m, 3H), 7.35-7.42 (m, 4H), 7.65 (dd, 1H, J=4, 8 Hz), 8.42 (br s, 1H), 8.55 (d, 1H, J=9 Hz), 8.92 (d, 1H, J=4H); ³¹P (121.4 MHz, DMSO-d6, 85° C.) δ 26.1, 28.3; MS (m/z) 636.5 [M+H]⁺.

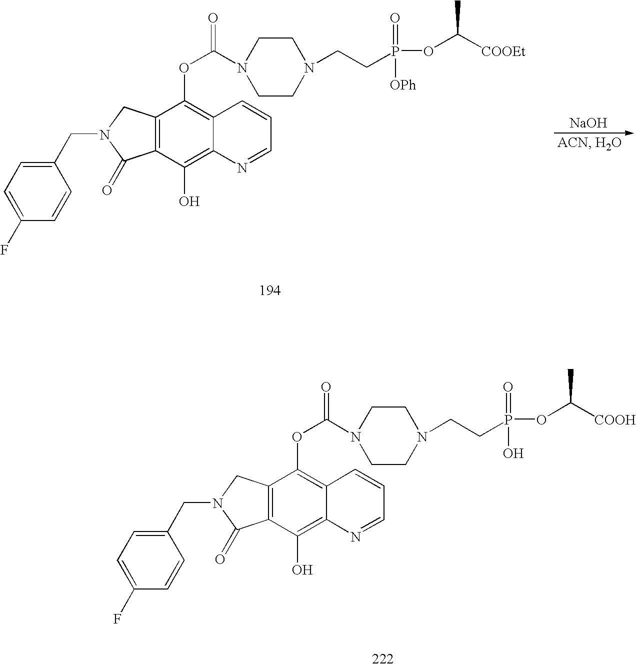

### Example 222

A solution of the trifluoroacetate salt of 4-{2-[(1-ethoxycarbonyl-ethoxy)-phenoxy-phosphoryl]-ethyl}-piperazine-1-carboxylic acid 7-(4-fluoro-benzyl)-9-hydroxy-8-oxo-7,8-dihydro-6H-pyrrolo[3,4-g]quinolin-5-yl ester 194 (0.045 g, 0.054 mmol) in acetonitrile (ACN, 0.68 mL) and water (0.68 mL) was treated with an aqueous solution of NaOH (0.162 mL, 1M). The reaction mixture was stirred at room temperature for 3 hours. The mixture was cooled to 0° C., then acidified with a 2N aqueous solution of HCl to pH=1. Acetonitrile was removed in vacuo then purified by reversed phase HPLC to afford the trifluoroacetate salt of 4-{2-[(1-carboxy-ethoxy)-hydroxy-phosphoryl]-ethyl}-piperazine-1-carboxylic acid 7-(4-fluoro-benzyl)-9-hydroxy-8-oxo-7,8-dihydro-6H-pyrrolo[3,4-g]quinolin-5-yl ester; compound with trifluoro-acetic acid 222 (0.032 g, 80%): ¹H NMR (CD₃OD) δ 9.0 (d, 1H), 8.5 (d, 1H), 7.75 (dd, 1H), 7.4 (dd, 2H), 7.1 (t, 2H), 4.8 (s, 2H), 4.45 (s, 2H), 4.3-3.7 (m, 4H), 3.7-3.35 (m, 6H), 2.2 (m, 2H), 1.55 (d, 3H); ³¹P NMR (CDCl₃) δ 19.8; MS: 617 (M+1).

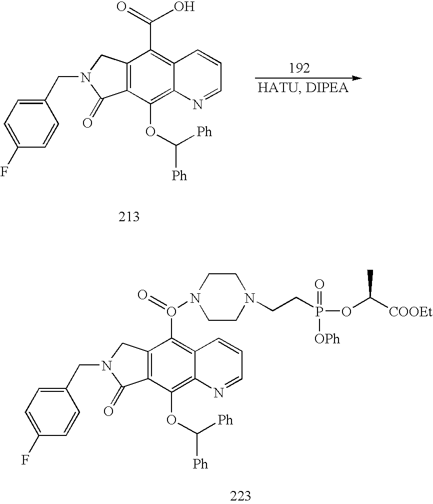

### Example 223

A solution of the 9-benzhydryloxy-7-(4-fluoro-benzyl)-8-oxo-7,8-dihydro-6H-pyrrolo[3,4-g]quinoline-5-carboxylic acid 213 (0.415 g, 0.80 mmol) and HATU (0.608 g, 1.60 mmol) in N,N-dimethylformamide (DMF) (2.5 mL) was stirred under an inert atmosphere at room temperature for 5 minutes. To the solution was added a premixed solution of 2-[phenoxy-(2-piperazin-1-yl-ethyl)-phosphinoyloxy]-(S)-propionic acid ethyl ester: compound with trifluoroacetic acid 192 (0.580 g, 1.20 mmol), N,N-Diisopropylethylamine (DIPEA) (0.700 mL, 4.0 mmol) in DMF (3.5 mL). The reaction mixture was stirred at room temperature for 5 hours. The mixture was diluted with ethyl acetate, washed with saturated NaHCO₃ (twice), water (twice) and brine (twice), dried (NaSO₄), and concentrated. The residue was purified by silica gel chromatography (5/95—methanol/methylene chloride) to afford 2-[(2-{4-[9-benzyhydryloxy-7-(4-fluoro-benzyl-8-oxo-7,8-dihydro-6H-pyrrolo[3,4-g]quinoline-5-carbonyl]-piperazin-1-yl}-ethyl)-phenoxy-phosphinoyloxy]-(S)-propionic acid ethyl ester 223 (0.625 g, 90%) as mixture of diastereomers: ¹H NMR (CDCl₃) δ 9.07 (dd, 1H), 8.15 (s, 1H), 8.05 (dd, 1H), 7.75 (d, 4H), 7.52 (dd, 1H), 7.4-7.1 (m, 13H), 7.05 (t, 2H), 5.02 (m, 1H), 5.0-4.6 (dd, 2H), 4.4-4.0 (dd, 2H), 4.17 (m, 2H), 4.0-3.5 (m, 3H), 3.0 (m, 2H), 2.7-2.5 (m, 3H), 2.4-2.1 (m, 4H), 1.6 & 1.4 (d, 3H), 1.25 (t, 3H); ³¹P NMR (CDCl₃) δ 28.3, 26.5; MS: 871 (M+1).

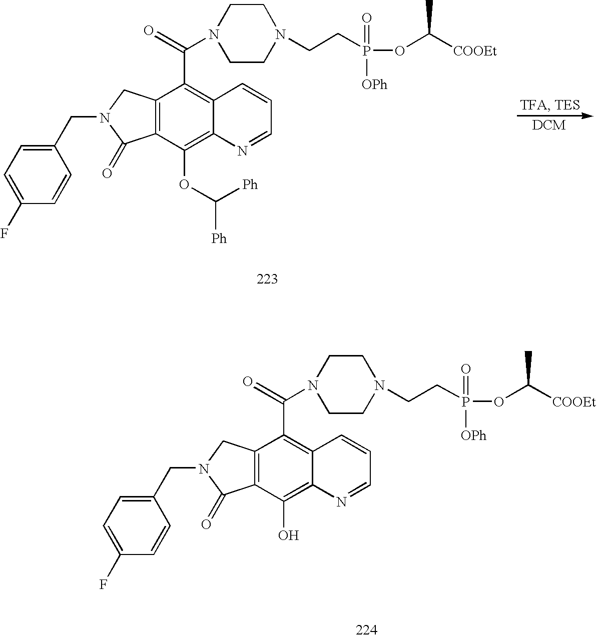

### Example 224

A solution of 2-[(2-{4-[9-benzyhydryloxy-7-(4-fluoro-benzyl-8-oxo-7,8-dihydro-6H-pyrrolo[3,4-g]quinoline-5-carbonyl]-piperazin-1-yl}-ethyl)-phenoxy-phosphinoyloxy]-propionic acid ethyl ester 223 (0.420 g, 0.483 mmol) in methylene chloride (2 mL) was treated with trifluoroacetic acid (0.4 mL) and triethylsilane (0.8 mL). The reaction mixture was stirred at room temperature under an inert atmosphere for 40 minutes. The volatiles were removed in vacuo with toluene. The product was triturated in diethyl ether/hexane with sonicaton to afford the trifluoroacetate salt of 2-{[2-4-2-[7-(4-fluoro-benzyl)-9-hydroxy-8-oxo-7,8-dihydro-6H-pyrrolo[3,4-g]quinolin-5-yl]-acetyl}-piperazin-1-yl)-ethyl]-phenoxy-phosphinoyloxy}-propionic acid ethyl ester 224 (0.370 g, 94%): ¹H NMR (CDCl₃) δ 9.0 (d, 1H), 8.15 (dd, 1H), 7.67 (dd, 1H), 7.35-7.1 (m, 7H), 7.05 (t, 2H), 5.0 (m, 1H), 5.0-4.6 (m, 2H), 4.6-4.25 (m, 2H), 4.25-3.95 (m, 5H), 3.7-2.8 (m, 8H), 2.7-2.5 (m, 2H), 1.6 & 1.4 (d, 3H), 1.25 (t, 3H); ³¹P NMR (CDCl₃) δ 23.0, 21.0; MS: 705 (M+1).

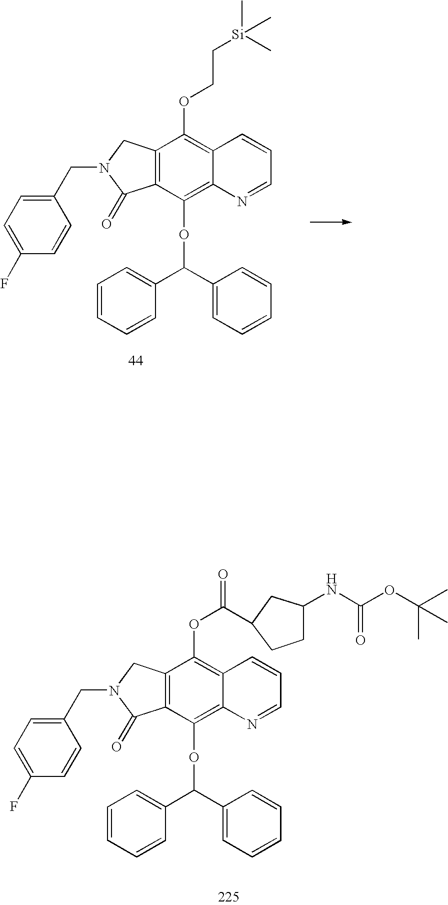

### Example 225

Trimethylsilylethyl ether 44 (0.03 g, 0.0508 mmol) was dissolved in 2 mL dry tetrahydrofuran. To this was added triethylamine (0.028 mL, 0.2032 mmol) and 1 M tetrabutylammonium fluoride solution in tetrahydrofuran (0.1016 mL, 0.1016 mmol.) Stirred at room temperature 10 minutes until starting material consumed. Diluted with dichloromethane, washed with washed with 1M HCl solution, saturated brine, concentrated to give crude. Dissolved in 1.5 mL dichloromethane, added catalytic dimethylaminopyridine, triethylamine (0.16 mL, 0.6 mmol) and cooled to 0° C. To this was added triphosgene (0.03 g, 0.1016 mmol) and stirred 40 minutes. BOC-aminopyrrolidine (0.038 g, 0.2032 mmol) was then added and stirred at room temperature for 10 minutes. The mixture was diluted with dichloromethane, washed with 1M HCl, brine, concentrated volatiles to give crude product. Chromatographed (10% to 30% acetone/toluene) to give 225 (0.0108 g, 0.0153 mmol, 30%.) ¹H NMR (CDCl₃) δ 9.03 (dd, 1H), 8.11 (d, 1H), 8.03 (s, 1H), 7.74 (d, 4H), 7.50 (dd, 1H), 7.27 (m, 8H), 7.07 (dd, 2H), 4.80 (s, 2H), 4.65 (br s, 1H), 4.30 (br s, 1H), 4.24 (s, 2H), 3.95 (br s, 1H), 3.74 (m, 2H), 3.58 (m, 2H), 1.48 (s, 9H) MS: 703 M+1)

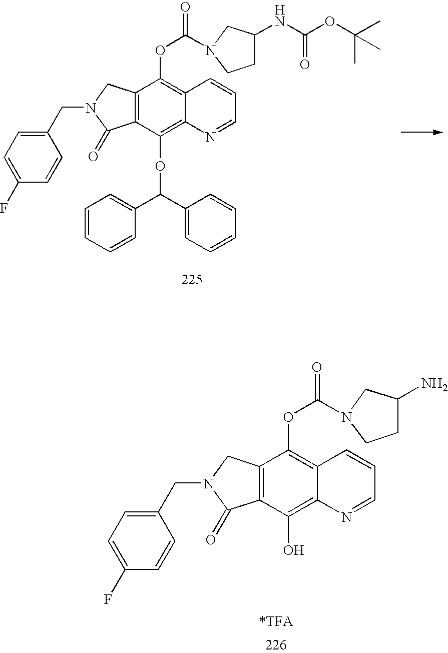

### Example 226

Carbamate 225 (0.0108 g, 0.0153 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Then dissolved in 0.3 mL dichloromethane, 0.3 ml trifluoroacetic acid. Stirred at room temperature for one hour. Concentrated off volatiles, azeotroped with toluene to give crude. Triturated twice with 1:1 diethyl ether/hexanes to give the trifluoroacetate salt of 3-amino-pyrrolidine-1-carboxylic acid 7-(4-fluoro-benzyl)-9-hydroxy-8-oxo-7,8-dihydro-6H-pyrrolo[3,4-g]quinolin-5-yl ester 226 (0.0057 g, 0.0104 mmol, 68%.) ¹H NMR (CD₃SOCD₃) δ 9.00 (s, 1H), 8.41 (s, 1H), 8.21 (s, 1H), 7.76 (dd, 1H), 7.36 (dd, 2H), 7.22 (dd, 2H), 4.72 (s, 2H), 4.36 (s, 2H), 3.93-3.35 (m, 7H) ¹⁹F NMR: −73.9 MS: 437 (M+1), 435 (M−1)

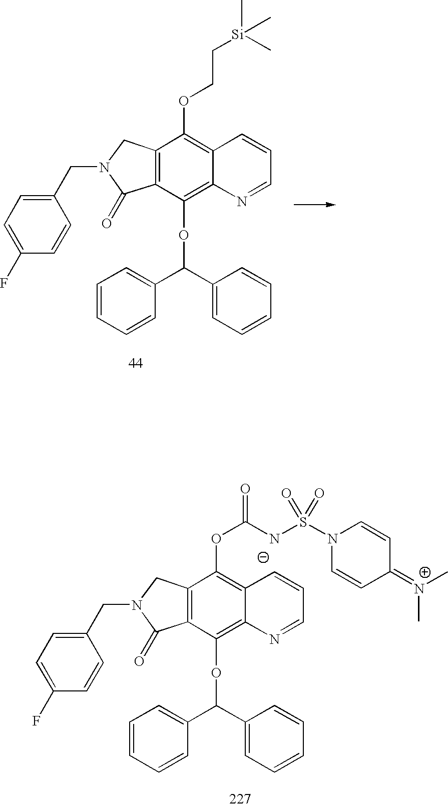

### Example 227

2-Amino-1,2,4 thiadiazole (0.006 g, 0.06 mmol) and triethylamine (0.0376 mL, 0.27 mmol) were added to 1 mL dichloromethane and cooled to 0° C. To this was slowly added chlorosulfonylisocyanate (0.007 mL, 0.08 mmol) at 0° C. Stirred thirty minutes until starting material consumed. Simultaneously, in a separate flask trimethylsilylethyl ether 44 was dissolved in 0.5 mL tetrahydrofuran. To this was added triethylamine (0.0376 mL, 0.27 mmol) and 1M tetrabutylammonium fluoride in tetrahydrofuran (0.135, 0.135 mmol) and stirred at room temperature. After 20 minutes, diluted with dichloromethane, washed with 1M HCl solution and brine, concentrated to give crude. At 0° C., dissolved in 0.5 mL dichloromethane and added to the solution prepared in situ above. Stirred at 0° C. for 5 minutes, catalytic DMAP added, then stirred for one hour at room temperature. Diluted with dichloromethane, washed with 1M HCl solution, brine, concentrated to give crude. Chromatographed (5 to 30% methanol/dichloromethane) to give dimethylaminopyridine adduct 227 (0.033 g, 0.046 mmol, 68%.) ¹H NMR (CDCl₃) δ 8.97 (dd, 1H), 8.54 (d, 2H), 8.19 (d, 1H), 8.00 (s, 1H), 7.72 (d, 4H), 7.42 (dd, 1H), 7.26-7.14 (m, 7H), 7.02 (dd, 2H), 6.52 (d, 2H), 4.74 (s, 2H), 4.17 (s, 2H), 3.22 (s, 6H.) MS: 718 (M+1).

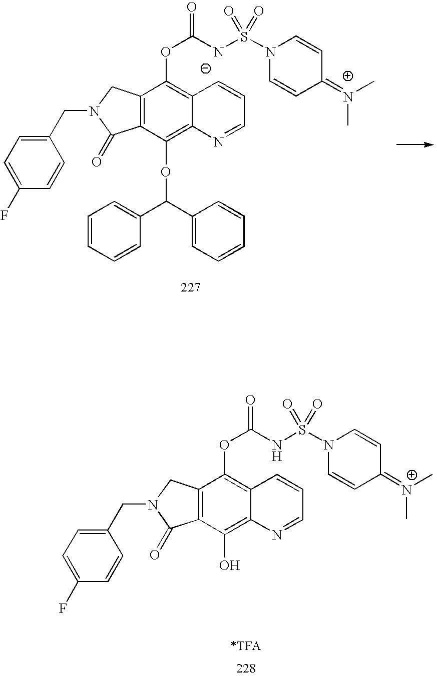

### Example 228

Carbamate 227 (0.007 gm, 0.0097 mmol) was dissolved in 0.25 mL of dichloromethane. To this was added 0.1 mL of triethylsilane and 0.05 mL of trifluoroacetic acid. Stirred at room temperature and after ten minutes complete by TLC. Concentrated off volatiles, azeotroped with toluene to give crude. Triturated twice with 1:1 diethyl ether/hexanes to give 228 (0.004 g, 0.0073 mmol, 75%.) ¹H NMR (CD₃SOCD₃) δ 9.22 (d, 1H), 9.09 (s, 1H), 8.47 (s, 1H), 8.19 (s, 1H), 8.01 (s, 1H), 7.37 (s, 2H), 7.19 (s, 1H), 6.96 (s, 2H), 4.76 (s, 2H), 4.45 (s, 2H), 3.21 (d, 6H.) ¹⁹F NMR: −75.95 MS: 552 (M+1), 550 (M−1)

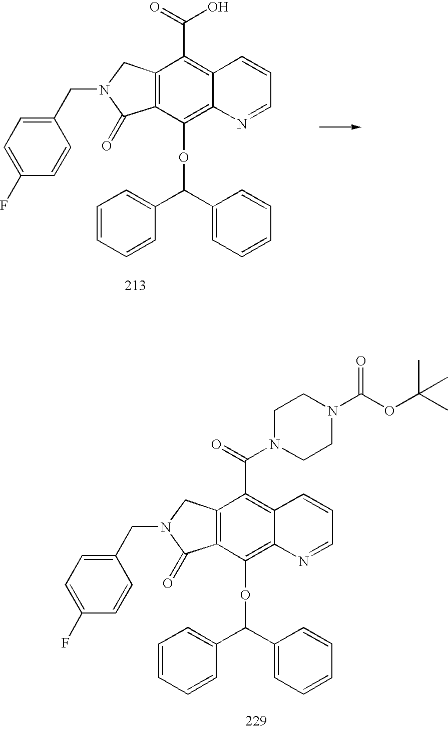

### Example 229

Carboxylic acid 213 (0.015 g, 0.029 mmol) was dissolved in 0.8 mL of dimethylformamide. To this was added BOC-piperazine (0.0116 g, 0.058 mmol), triethylamine (0.012 mL, 0.087 mmol), 1-(3-Dimethylaminopropyl)-3-ethylcarbodiimide hydrochloride (0.011 g, 0.058 mmol), 1-Hydroxybenzotriazole hydrate (0.0059 g, 0.0435 mmol) and stirred at room temperature. After 15 hours, starting material was consumed. Dilute with dichloromethane, washed with 1M HCl solution, saturated brine solution, dried (Na₂SO₄), concentrated to give crude product. Chromatographed (10 to 50% ethyl acetate/hexanes) to give 4-[9-benzhydryloxy-7-(4-fluoro-benzyl)-8-oxo-7,8-dihydro-6H-pyrrolo[3,4-g]quinoline-5-carbonyl]-piperazine-1-carboxylic acid tert-butyl ester 229 (0.009 g, 0.013 mmol, 45%.) ¹H NMR (CDCl₃) 9.075 (s, 1H), 8.15 (s, 1H), 8.03 (d, 1H), 7.74 (dd, 4H), 7.53 (dd, 1H), 7.27 (m, 8H), 7.04 (dd, 2H), 4.91 (d, J=17 Hz, 1H), 4.69 (d, J=17 Hz, 1H), 4.41 (d, J=17 Hz, 1H), 4.055 (d, J=17 Hz, 1H), 3.55-2.96 (br m, 8H), 1.44 (s, 9H.) MS: 687 (M+1).

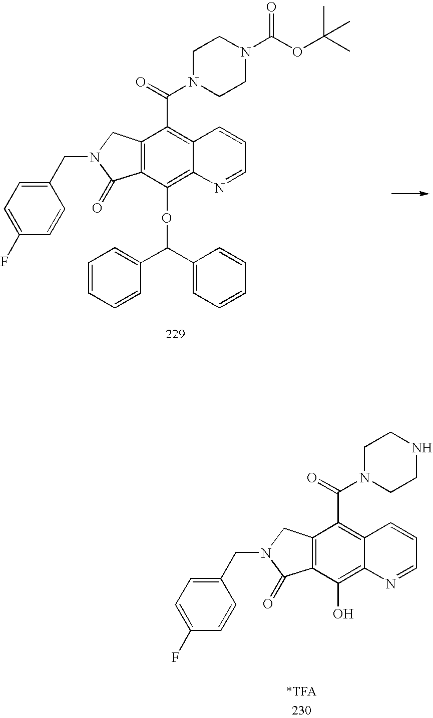

### Example 230

Carboxamide 229 (0.0108 g, 0.0153 mmol) was dissolved in 1 mL of dichloromethane. To this was added 0.4 mL of triethylsilane and 0.2 mL of trifluoroacetic acid. Stirred at room temperature and after ten minutes complete by TLC. Concentrated off volatiles, azeotroped with toluene to give crude. Then dissolved in 0.6 mL dichloromethane, 0.6 ml trifluoroacetic acid. Stirred at room temperature for one hour. Concentrated off volatiles, azeotroped with toluene to give crude. Triturated twice with 1:1 diethyl ether/hexanes to give 7-(4-fluoro-benzyl)-9-hydroxy-5-(piperazine-1-carbonyl)-6,7-dihydro-pyrrolo[3,4-g]quinolin-8-one 230 (0.039 g, 0.0682 mmol, 100%.) ¹H NMR (CD₃SOCD₃) δ 98.97 (s, 2H), 8.32 (d, 1H), 7.74 (s, 1H), 7.36 (dd, 2H), 7.19 (dd, 2H), 4.86 (d, 1H), 4.58 (d, 1H), 4.42 (d, 1H), 4.34 (d, 1H), 3.9-2.90 (m, 8H.) ¹⁹F NMR: −74.202 MS: 421 (M+1), 419 (M−1)

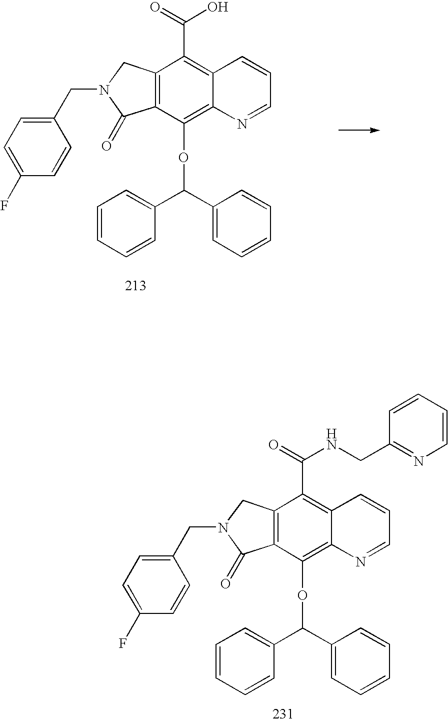

### Example 231

Carboxylic acid 213 (0.010 g, 0.0193 mmol) was dissolved in 0.3 mL of dimethylformamide. To this was added 2-aminomethylpyridine (0.004 g, 0.0386 mmol), triethylamine (0.008 mL, 0.058 mmol), 1-(3-Dimethylaminopropyl)-3-ethylcarbodiimide hydrochloride (0.074 g, 0.0386 mmol), 1-Hydroxybenzotriazole hydrate (0.0039 g, 0.029 mmol) and stirred at room temperature. After 15 hours, starting material was consumed. Dilute with dichloromethane, washed with 1M HCl solution, saturated brine solution, dried (Na₂SO₄), concentrated to give crude product. Chromatographed (O to 8% methanol/dichloromethane) to give 9-benzhydryloxy-7-(4-fluoro-benzyl)-8-oxo-7,8-dihydro-6H-pyrrolo[3,4-g]quinoline-5-carboxylic acid (pyridin-2-ylmethyl)-amide 231 (0.007 g, 0.011 mmol, 59%.) ¹H NMR (CDCl₃) 8.94 (s, 1H), 8.45 (d, 2H), 8.05 (s, 1H), 7.70 (d, 4H), 7.57-7.17 (m, 12H), 7.05 (d, 2H), 4.78 (s, 1H), 4.69 (d, J=5 Hz, 1H), 4.38 (s, 1H). MS: 609 (M+1).

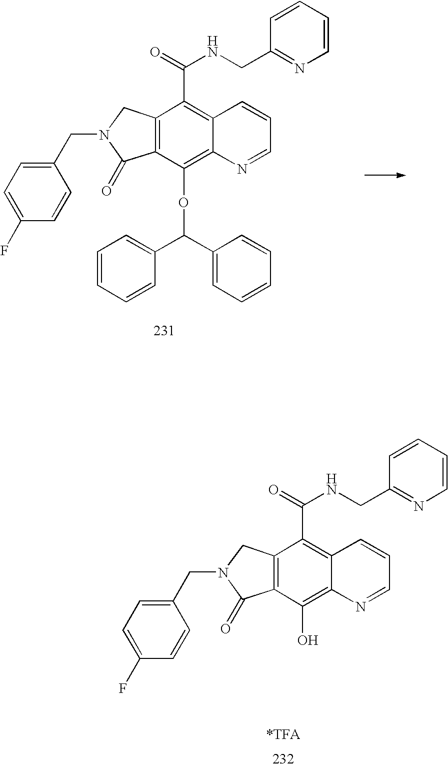

### Example 232

Carboxamide 231 (0.225 g, 0.355 mmol) was dissolved in 1 mL of dichloromethane. To this was added 0.5 mL of triethylsilane and 0.25 mL of trifluoroacetic acid. Stirred at room temperature and after ten minutes complete by TLC. Concentrated off volatiles, azeotroped with toluene to give crude. Triturated twice with 1:1 diethyl ether/hexanes to give 7-(4-fluoro-benzyl)-9-hydroxy-8-oxo-7,8-dihydro-6H-pyrrolo[3,4-g]quinoline-5-carboxylic acid (pyridin-2-ylmethyl)-amide 232 (0.11 g, 0.20 mmol, 56%.) ¹H NMR (CD₃SOCD₃) δ 9.18 (s, 1H), 8.96 (d, 1H), 8.65 (dd, 2H), 8.09 (dd, 1H), 7.76 (dd, 1H), 7.64 (dd, 1H), 7.36 (dd, 2H), 7.22 (dd, 2H), 4.70 (s, 4H), 4.54 (s, 2H). ¹⁹F NMR: −75.37 MS: 443 (M+1), 441 (M−1)

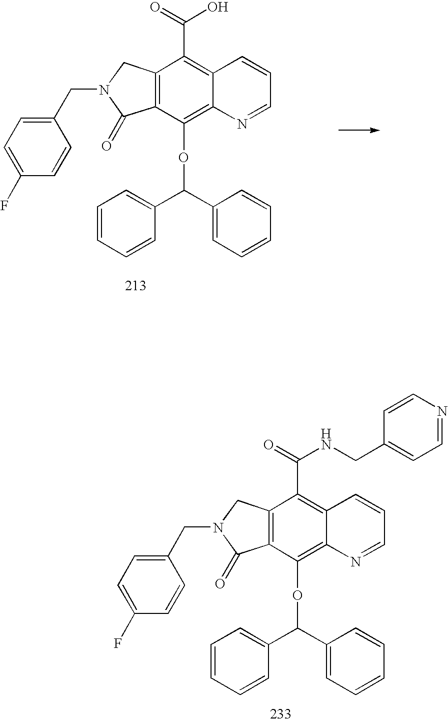

### Example 233

Carboxylic acid 213 (0.010 g, 0.0193 mmol) was dissolved in 0.3 mL of dimethylformamide. To this was added 4-aminomethylpyridine (0.004 mL, 0.0386 mmol), triethylamine (0.008 mL, 0.058 mmol), 1-(3-Dimethylaminopropyl)-3-ethylcarbodiimide hydrochloride (0.074 g, 0.0386 mmol), 1-Hydroxybenzotriazole hydrate (0.0039 g, 0.029 mmol) and stirred at room temperature. After 15 hours, starting material was consumed. Dilute with dichloromethane, washed with 1M HCl solution, saturated brine solution, dried (Na₂SO₄), concentrated to give crude product. Chromatographed (0 to 8% methanol/dichloromethane) to give 233 (0.0048 g, 0.008 mmol, 41%.) ¹H NMR (CDCl₃) δ 8.71 (s, 1H), 8.66 (d, 2H), 7.99 (dd, 2H), 7.65 (s, 1H), 7.51 (s, 4H), 7.34 (m, 9H), 7.05 (dd, 2H), 4.69 (s, 2H), 4.25 (d, 2H), 4.00 (s, 2H). MS: 609 (M+1).

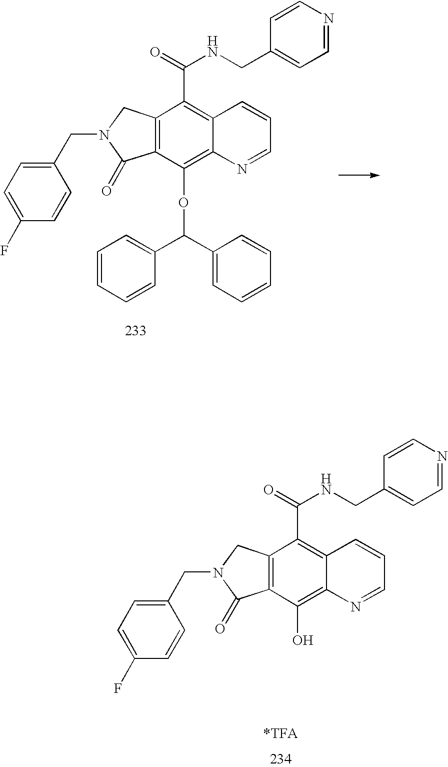

### Example 234

Carboxamide 233 (0.137 g, 0.225 mmol) was dissolved in 1 mL of dichloromethane. To this was added 0.5 mL of triethylsilane and 0.25 mL of trifluoroacetic acid. Stirred at room temperature and after ten minutes complete by TLC. Concentrated off volatiles, azeotroped with toluene to give crude. Triturated twice with 1:1 diethyl ether/hexanes to give 7-(4-fluoro-benzyl)-9-hydroxy-8-oxo-7,8-dihydro-6H-pyrrolo[3,4-g]quinoline-5-carboxylic acid (pyridin-4-ylmethyl)-amide 234 (0.114 g, 0.20 mmol, 91%.) ¹H NMR (CD₃SOCD₃) δ 9.24 (dd, 1H), 8.98 (d, 1H), 8.77 (dd, 2H), 8.53 (d, 1H), 7.79 (dd, 3H), 7.40 (dd, 2H), 7.23 (dd, 2H), 4.71 (s, 4H), 4.56 (s, 2H). ¹⁹F NMR: −74.906 MS: 443 (M+1), 441 (M−1)

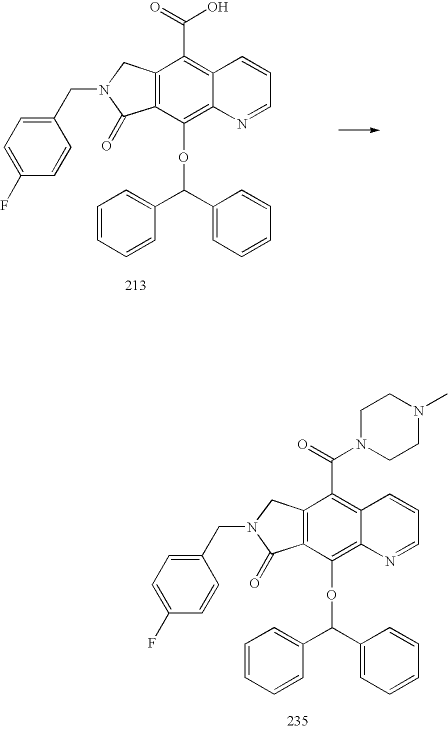

### Example 235

Carboxylic acid 213 (0.020 g, 0.0386 mmol) was dissolved in 0.4 mL of dimethylformamide. To this was added methyl piperazine (0.0085 mL, 0.077 mmol), diisopropylethylamine (0.027 mL, 0.154 mmol), O-(7-Azabenzotriazol-1-yl)-N,N,N′,N′-tetramethyluronium hexafluorophosphate (0.029 g, 0.0777 mmol) and stirred at room temperature. After 15 hours, starting material was consumed. Dilute with dichloromethane, washed with saturated brine solution, dried (Na₂SO₄), concentrated to give crude product. Chromatographed (0 to 8% methanol/dichloromethane) to give 235 (0.017 g, 0.028 mmol, 73%.) ¹H NMR (CDCl₃) δ 9.06 (dd, 1H), 8.13 (s, 1H), 8.05 (dd, 1H), 7.76 (dd, 4H), 7.53 (dd, 1H), 7.27 (m, 8H), 7.06 (dd, 2H), 4.93 (d, J=15 Hz, 1H), 4.72 (d, J=15 Hz, 1H), 4.36 (d, J=15 Hz, 1H), 4.066 (d, J=15 Hz, 1H), 3.88-2.97 (m, 8H), 2.28 (s, 3H.) MS: 601 (M+1).

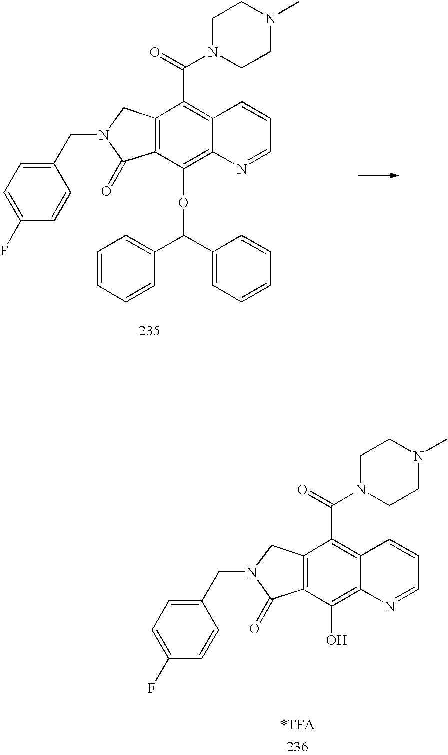

### Example 236

Carboxamide 235 (0.015 g, 0.025 mmol) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethyl ether/hexanes to give 7-(4-fluoro-benzyl)-9-hydroxy-5-(4-methyl-piperazine-1-carbonyl)-6,7-dihydro-pyrrolo[3,4-g]quinolin-8-one 236 (0.0135 g, 0.227 mmol, 91%.) ¹H NMR 90° C.(CD₃SOCD₃) δ 8.98 (dd, 1H), 8.28 (d, 1H), 7.74 (dd, 1H), 7.40 (dd, 2H), 7.21 (dd, 2H), 4.72 (s, 4H), 4.40 (s, 4H), 3.5 (br s, 4H), 2.81 (s, 3H.) ¹⁹F NMR: −74.688 MS: 436 (M+1), 434 (M−1)

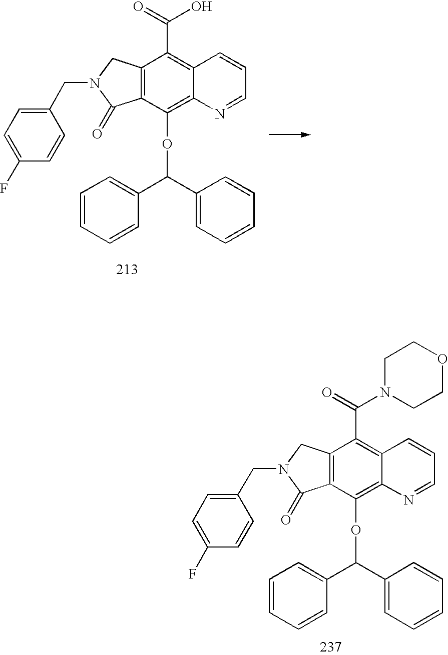

### Example 237

Carboxylic acid 213 (0.10 g, 0.193 mmol) was dissolved in 2 mL of dimethylformamide. To this was added morpholine (0.0337 mL, 0.386 mmol), diisopropylethylamine (0.135 mL, 0.772 mmol), O-(7-azabenzotriazol-1-yl)-N,N,N′,N′-tetramethyluronium hexafluorophosphate (HATU, 0.146 g, 0.386 mmol) and stirred at room temperature. After 15 hours, starting material was consumed. Dilute with dichloromethane, washed with 1M HCl solution, saturated brine solution, dried (Na₂SO₄), concentrated to give crude product. Chromatographed (O to 5% methanol/dichloromethane) to give pure product (0.06 g, 0.102 mmol, 53%.) ¹H NMR (CDCl₃) δ 9.08 (dd, 1H), 8.15 (s, 1H), 8.06 (dd, 1H), 7.76 (dd, 4H), 7.55 (dd, 1H), 7.30 (m, 8H), 7.07 (dd, 2H), 4.95 (d, J=15 Hz, 1H), 4.70 (d, J=15 Hz, 1H), 4.42 (d, J=15 Hz, 1H), 4.14 (d, J=15 Hz, 1H), 3.94-3.79 (m, 4H), 3.41 (m, 2H), 2.99 (m, 2H.) MS: 588 (M+1).

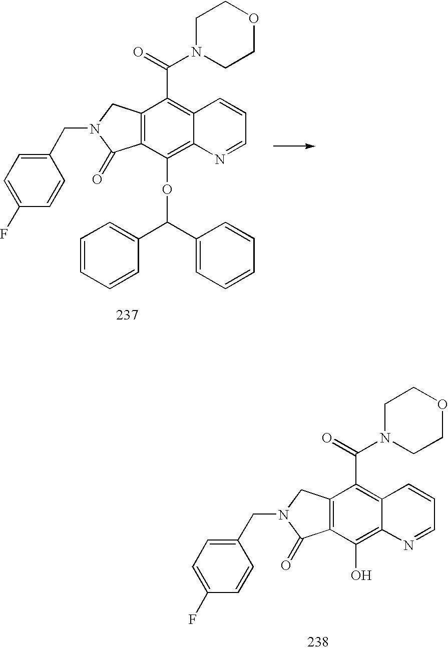

### Example 238

Carboxamide 237 (0.06 g, 0.102 mmol) was dissolved in 1 mL of dichloromethane. To this was added 0.4 mL of triethylsilane and 0.2 mL of trifluoroacetic acid. Stirred at room temperature and after ten minutes complete by TLC. Concentrated off volatiles, azeotroped with toluene to give crude. Triturated twice with 1:1 diethyl ether/hexanes to give 7-(4-fluoro-benzyl)-9-hydroxy-5-(morpholine-4-carbonyl)-6,7-dihydro-pyrrolo[3,4-g]quinolin-8-one 238 (0.0459 g, 0.09 mmol, 100%.) ¹H NMR (CDCl₃) δ 9.05 (dd, 1H), 8.20 (d, 1H), 7.64 (dd, 1H), 7.35 (m, 2H), 7.08 (dd, 2H), 4.91 (d, J=15 Hz, 1H), 4.68 (d, J=15 Hz, 1H), 4.59 (d, J=15 Hz, 1 Hz), 4.24 (d, J=15 Hz, 1H), 3.99 (m, 3H), 3.5 (s, 2H), 3.18 (s, 2H.) MS: 436 (M+1), 434 (M−1)

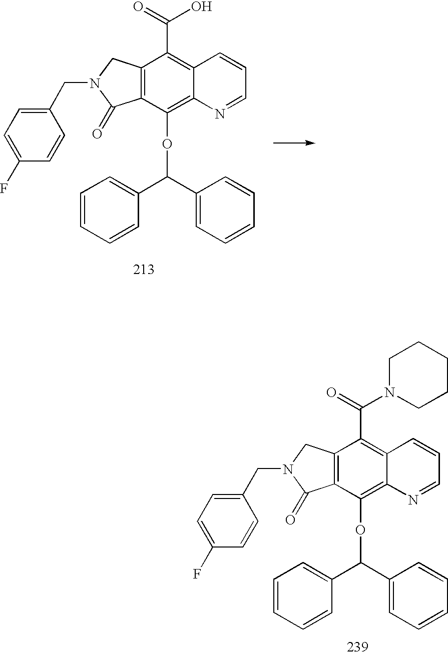

### Example 239

Carboxylic acid 213 (0.018 g, 0.0347 mmol) was dissolved in 0.5 mL of dimethylformamide. To this was added piperidine (0.0068 mL, 0.0695 mmol), diisopropylethylamine (0.024 mL, 0.139 mmol), HATU (0.027 g, 0.0695 mmol) and stirred at room temperature. After 2.5 hours, starting material was consumed. Dilute with ethyl acetate, washed with 2.5% LiCl solution, saturated brine solution, dried (Na₂SO₄), concentrated to give crude 239. ¹H NMR (CDCl₃) δ 9.04 (dd, 1H), 8.12 (s, 1H), 8.06 (d, 1H), 7.75 (dd, 4H), 7.52 (dd, 1H), 7.30 (m, 8H), 7.06 (dd, 2H), 4.94 (d, J=15 Hz, 1H), 4.69 (d, J=15 Hz, 1H), 4.40 (d, J=15 Hz, 1H), 4.07 (d, J=15 Hz, 1H), 3.91 (s, 1H), 3.71 (s, 1H), 3.28 (s, 1H), 3.18 (s, 1H), 2.0-1.28 (m, 6H.) MS: 586 (M+1).

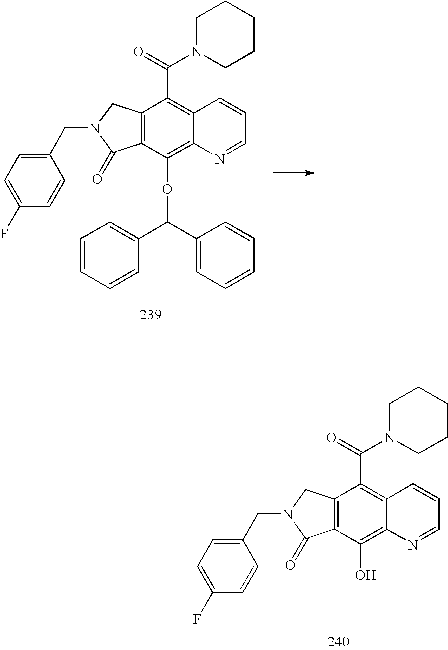

### Example 240

Carboxamide 239 (crude) was dissolved in 0.5 mL of dichloromethane. To this was added 0.2 mL of triethylsilane and 0.1 mL of trifluoroacetic acid. Stirred at room temperature and after ten minutes complete by TLC. Concentrated off volatiles, azeotroped with toluene to give crude. Triturated twice with 1:1 diethyl ether/hexanes to give 7-(4-fluoro-benzyl)-9-hydroxy-5-(piperidine-1-carbonyl)-6,7-dihydro-pyrrolo[3,4-g]quinolin-8-one 240 (0.0084 g, 0.02 mmol, 58% for 2 steps.) ¹H NMR (CDCl₃) δ 8.97 (dd, 1H), 8.17 (d, 1H), 7.60 (dd, 1H), 7.34 (dd, 2H), 7.07 (dd, 2H), 4.91 (d, J=15 Hz, 1H), 4.66 (d, J=15 Hz, 1H), 4.56 (d, J=15 Hz, 1 Hz), 4.22 (d, J=15 Hz, 1H), 3.91 (s, 1H), 3.75 (s, 1H), 3.11 (s, 2H), 1.7-1.3 (m, 6H.) MS: 420 (M+1), 418 (M−1)

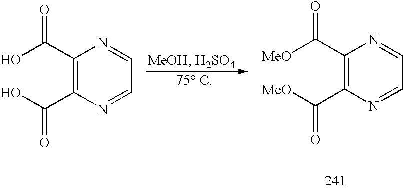

### Example 241

To a mixture of pyrazine-2,3-dicarboxylic acid (20 g, 119 mmol, 1 equiv.) was added MeOH (80 mL) followed by dropwise addition of concentrated H₂SO₄ (36 mL, 680 mmol, 5.7 equiv.) over 45 minutes. This method is similar to that that cited for a different substrate (J. Am. Chem. Soc., 73, 1951, 5614-5616). The reaction was heated at 75° C. for 16 hours and then cooled and quenched with water (200 mL). It was extracted with EtOAc (4×60 mL) and the organic layer washed several times with water (3×50 ml), saturated NaHCO₃ (50 ml), brine solution (50 mL). It was dried over Na₂SO₄, filtered and concentrated in vacuo to yield pyrazine-2,3-dicarboxylic acid methyl ester 241 as a brown solid (47%, 10.97 g, 55.9 mmol). ¹H NMR (300 MHz) CDCl₃ δ 8.79 (d, J=2.7 Hz, 2H), 4.05 (s, 3H), 4.04 (s, 3H). TLC R^{f}: 0.7 ethyl acetate/methanol (9/1)

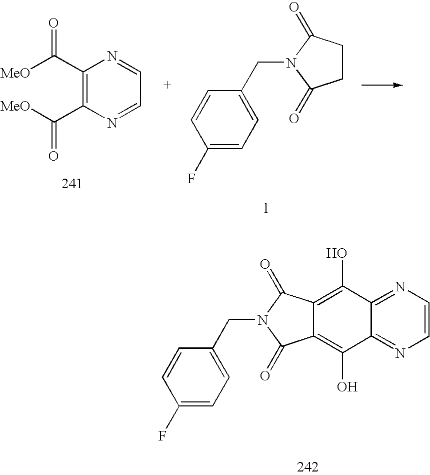

### Example 242

Into a flask containing pyrazine-2,3-dicarboxylic acid methyl ester 241 (10.70 g, 54.6 mmol, 1 equiv.) was added THF (150 mL) under a nitrogen atmosphere followed by 1-(4-Fluoro-benzyl)-pyrrolidine-2,5-dione 1 (11.30 g, 54.6 mmol, 1 equiv.). MeOH (1.8 mL) was then added and at 0° C. was added NaH (4.8 g, 120.1 mmol, 2.2 equiv.) carefully in four portions. Refluxing was carried out for 20 hours after which the reaction was cooled and placed in a 0° C. icebath. HCl (6 N, 30 mL, H₂O) was slowly added while vigorously stirring. The resulting solid was filtered, and washed thoroughly with water followed by ether. It was then dried in a vacuum oven (60° C., 12 hours) to realize 8.7 gm (47%, 25.66 mmol) of 7-(4-fluoro-benzyl)-5,9-dihydroxy-pyrrolo[3,4-g]quinoxaline-6,8-dione 242. ¹H NMR (300 MHz) CDCl₃ δ 7.15-7.33 (m, 5H), 5.91 (s, 2H), 3.96 (s, 3H), 3.88 (s, 3H). MS: 340.3 (M+1).

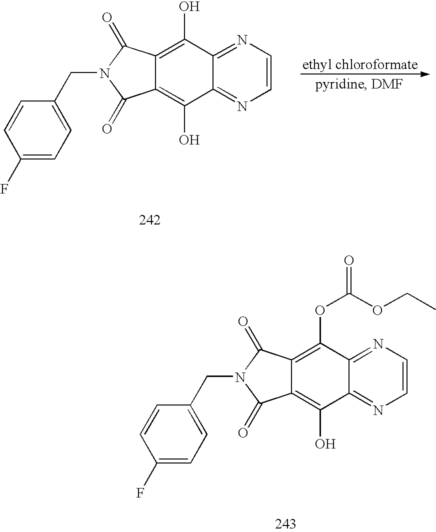

### Example 243

7-(4-Fluoro-benzyl)-5,9-dihydroxy-pyrrolo[3,4-g]quinoxaline-6,8-dione 242 (1 g, 2.95 mmol, 1 equiv.) was dissolved in DMF (30 ml, 0.1 M) and pyridine (477 μL, 5.89 mmol, 2 equiv.) before ethyl chloroformate was added (237 μL, 2.95 mmol, 1 equiv.). The reaction was stirred for 16 hours before being quenched with HCl (30 ml, 1 N) and extracted with ethyl acetate (2×30 mL). The organic layer washed several times with water (4×30 mL), saturated NaHCO₃ (50 mL), brine solution (50 mL). It was dried over Na₂SO₄, filtered and concentrated in vacuo. Recrystallization was carried out in ethyl acetate and Hexanes to yield carbonic acid ethyl ester 7-(4-fluoro-benzyl)-9-hydroxy-6,8-dioxo-7,8-dihydro-6H-pyrrolo[3,4-g]quinoxalin-5-yl ester 243 as a light brown solid (98%, 1.20 g, 2.89 mmol). ¹H NMR (300 MHz) CDCl₃ δ 9.09 (d, J=6 Hz, 1H), 8.97 (d, J=6 Hz, 1H), 8.65 (bs, 1H), 7.46 (d, J=4.8 Hz, 2H), 7.03 (d, J=4.8 Hz, 2H), 4.85 (s, 2H), 4.04 (q, J=2.8 Hz, 2H), 1.43 (q, J=2.8 Hz, 3H). MS: 412.6 (M+1).

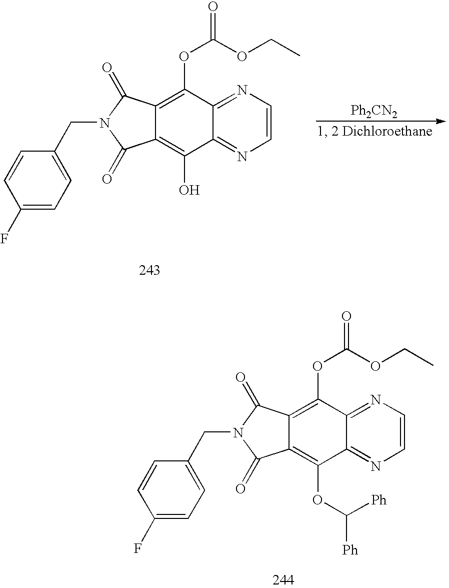

### Example 244

Carbonic acid mono-[1-(1-benzyl-4-methylene-2,5-dioxo-pyrrolidin-3-ylidene)-ethyl] ester 243 (1.1 g, 2.68 mmol, 1 equiv.) was dissolved in 1,2 dichloroethane (50 mL, 0.055 M) and to this was added diphenyldiazomethane (1.05 g, 5.35 mmol, 2 equiv.) and heated at 70° C. under a nitrogen atmosphere for 24 hours. The reaction was concentrated in vacuo and purified by silica gel chromatography using 4/1 Hexanes/Ethyl acetate to obtain carbonic acid 9-benzhydryloxy-7-(4-fluoro-benzyl)-6,8-dioxo-7,8-dihydro-6H-pyrrolo[3,4-g]quinoxalin-5-yl ethyl ester 244 (70%, 1085 mg, 1.87 mmol). ¹H NMR (300 MHz) CDCl₃ δ 9.09 (d, J=6 Hz, 1H), 8.97 (d, J=6 Hz, 1H), 8.65 (bs, 1H), 7.46 (d, J=4.8 Hz, 2H), 7.03 (d, J=4.8 Hz, 2H), 4.85 (s, 2H), 4.04 (q, J=2.8 Hz, 2H), 1.43 (q, J=2.8 Hz, 3H). MS: 600.2 (M+23). TLC R_{f}: 0.3 Hexanes/Ethyl acetate (7/3)

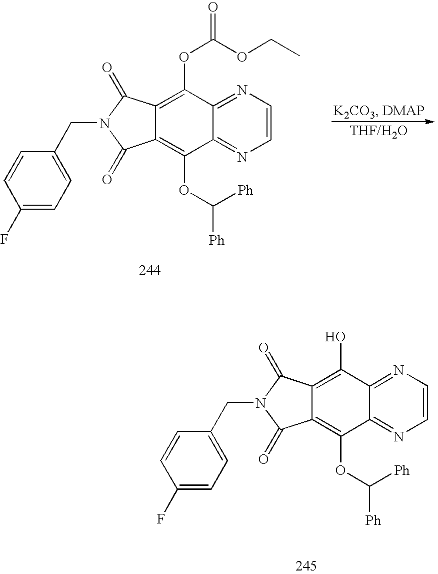

### Example 245

Carbonic acid 9-benzhydryloxy-7-(4-fluoro-benzyl)-6,8-dioxo-7,8-dihydro-6H-pyrrolo[3,4-g]quinoxalin-5-yl ester ethyl ester 244 (500 mg, 0.87 mmol) was dissolved in THF (9 mL, 0.1 M) along with DMAP (211 mg, 1.73 mmol, 2 equiv.). A solution of K₂CO₃ (1.20 g, 8.66 mmol, and 10 equiv.) was dissolved separately in H₂O (6 mL) before being transferred to the reaction mixture. The reaction was allowed to stir for 18 hours and quenched with HCl (20 mL, 1 N) and extracted with ethyl acetate (2×30 mL). The organic layer was washed with saturated NH₄Cl solution (25 mL), brine solution (25 mL) and dried over Na₂SO₄ and concentrated in vacuo to yield 5-benzhydryloxy-7-(4-fluoro-benzyl)-9-hydroxy-pyrrolo[3,4-g]quinoxaline-6,8-dione 245 (94%, 413 mg, 0.82 mmol).

¹H NMR (300 MHz) CDCl₃ δ 9.08 (d, J=1.5 Hz, 1H), 8.92 (d, J=1.5 Hz, 1H), 7.67 (s, 1H), 7.67-7.42 (dd, J=1.5 Hz, J₂=8.4 Hz, 4H), 7.43-7.48 (m, 2H), 7.19-7.27 (m, 7H), 7.03-7.20 (m, 1H), 4.86 (s, 2H). MS: 528.0 (M+23). TLC R_{f}: 0.2 Hexanes/Ethyl acetate (8/2)

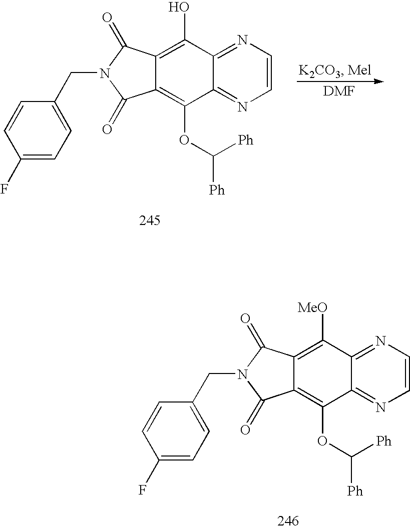

### Example 246

Into a flask containing 5-benzhydryloxy-7-(4-fluoro-benzyl)-9-hydroxy-pyrrolo[3,4-g]quinoxaline-6,8-dione 245 (350 mg, 0.69 mmol, 1 equiv.) was added DMF (20 mL) followed by K₂CO₃ (478 mg, 3.46 mmol, 5 equiv.). To this was added MeI (983 μL, 6.93 mmol, 10 equiv.) under a nitrogen atmosphere and stirred for 16 hours. To the reaction was then added water (50 mL) and extracted with ethyl acetate (2×40 mL). The organic layer was washed several times with water (3×30 mL), saturated NaHCO₃ (40 mL), brine solution (30 mL). It was dried over Na₂SO₄, filtered and concentrated in vacuo before being purified by silica gel chromatography using 3/2 Hexanes/ethyl acetate to obtain 5-benzhydryloxy-7-(4-fluoro-benzyl)-9-methoxy-pyrrolo[3,4-g]quinoxaline-6,8-dione 246 (78%, 280 mg, 0.54 mmol) as a yellow solid. ¹H NMR (300 MHz) CDCl₃ δ 9.03 (d, J=1.5 Hz, 1H), 8.97 (d, J=1.5 Hz, 1H), 7.75 (s, 1H), 7.60 (dd, J=1.5 Hz, J₂=8.4 Hz, 4H), 7.43-7.48 (m, 2H), 7.19-7.27 (m, 7H), 7.03-7.20 (m, 1H), 4.86 (s, 2H), 4.37 (s, 3H). MS: 542.0 (M+23). TLC R_{f}: 0.5 Hexanes/Ethyl acetate (1/1)

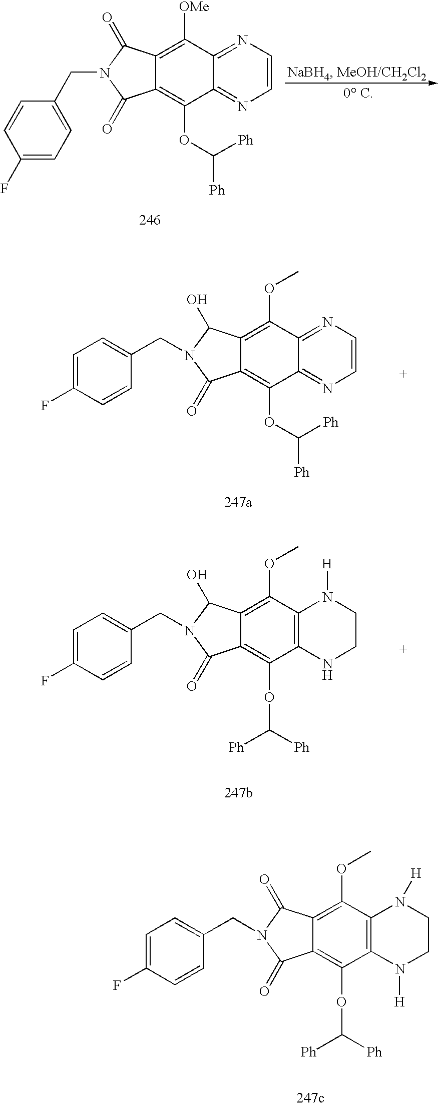

### Example 247

5-Benzhydryloxy-7-(4-fluoro-benzyl)-9-methoxy-pyrrolo[3,4-g]quinoxaline-6,8-dione 246 (10 mg, 0.019 mmol, 1 equiv.) was dissolved in CH₂Cl₂ (0.2 mL) and MeOH (0.5 mL) under a nitrogen atmosphere at 0° C. Sodium borohydride (NaBH₄) was added (115 μL, 0.057 mmol, 3 equiv., 0.5 M). The reaction was allowed to stir for 1 hour and then quenched with water (5 mL) and extracted with ethyl acetate (2×5 mL). The organic layer was washed several times with water (2×10 mL), brine solution (10 mL). It was dried over Na₂SO₄, filtered and concentrated in vacuo and purified by preparatory thin-layer chromatography (PTLC) using 3/2 Hexanes/Ethyl acetate to obtain 5-benzhydryloxy-7-(4-fluoro-benzyl)-8-hydroxy-9-methoxy-7,8-dihydro-pyrrolo[3,4-g]quinoxalin-6-one 247a (34%, 3 mg) and reduced species: 5-benzhydryloxy-7-(4-fluoro-benzyl)-8-hydroxy-9-methoxy-1,2,3,4,7,8-hexahydro-pyrrolo[3,4-g]quinoxalin-6-one 247b (21%, 2 mg) and 5-benzhydryloxy-7-(4-fluoro-benzyl)-9-methoxy-1,2,3,4-tetrahydro-pyrrolo[3,4-g]quinoxaline-6,8-dione 247c (34%, 3.4 mg).

247a: ¹H NMR (300 MHz) CDCl₃ δ 8.86 (d, J=1.8 Hz, 1H), 8.82 (d, J=1.8 Hz, 1H), 7.69 (s, 1H), 7.69-7.56 (m, 1H), 7.54-7.56 (m, 1H), 7.16-7.32 (m, 10H), 7.01-7.17 (s, 2H), 5.78 (bs, 1H), 5.18 (d, J=14.7 Hz, 1H), 4.38 (d, J=13.5 Hz, 1H), 4.18 (s, 3H), 3.83 (s, 2H). MS: 544.0 (M+23). TLC R_{f}: 0.3 Hexanes/Ethyl acetate (3/2)

247b: ¹H NMR (300 MHz) CDCl₃ δ 7.27-7.7.40 (m, 12H), 6.95-7.01 (m, 2H), 4.70 (s, 2H), 4.01 (s, 3H), 3.32 (t, J=3.9 Hz, 2H), 3.13 (t, J=5.1 Hz, 2H), 2.75 (s, 2H). MS: 545.9 (M+23). TLC R_{f}: 0.25 Hexanes/Ethyl acetate (1/1) 247c: ¹H NMR (300 MHz) CDCl₃ δ 7.27-7.7.40 (m, 12H), 5.58 (bs, 1H), 5.01 (d, J=14.1 Hz, 1H), 4.21 (d, J=9.6 Hz, 1H), 3.85 (s, 3H), 3.32-3.45 (m, 2H), 3.02-3.05 (t, J=5.1 Hz, 2H), 1.63 (bs, 2H). R_{f}: 0.2 Hexanes/Ethyl acetate (1/1)

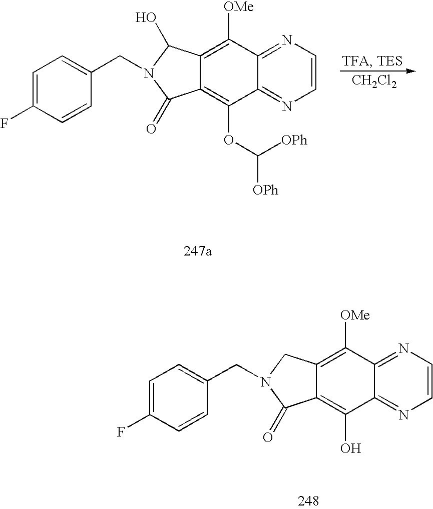

### Example 248

Into a flask containing 5-benzhydryloxy-7-(4-fluoro-benzyl)-8-hydroxy-9-methoxy-7,8-dihydro-pyrrolo[3,4-g]quinoxalin-6-one 247a (20 mg, 0.038 mmol, 1 equiv.) was added CH₂Cl₂ (1 mL) under a nitrogen atmosphere. Triethylsilane (200 μL) was added followed by trifluoroacetic acid (200 μL). The reaction was allowed to stir for 1 hour and then concentrated in vacuo until thoroughly dried. To the oil was Hexanes/Ethyl ether (15 mL, 1/1 ratio) and sonicated. The resulting solid was then filtered, washed in hexanes, and air dried to give 7-(4-fluoro-benzyl)-5-hydroxy-9-methoxy-7,8-dihydro-pyrrolo[3,4-g]quinoxalin-6-one 248 (38%, 7.2 mg, 0.0.14 mmol). ¹H NMR (300 MHz) CDCl₃ δ 8.95 (d, J=13.8 Hz, 2H), 7.23-7.27 (m, 2H), 6.96-7.05 (s, 2H), 4.79 (2H), 4.55 (s, 2H), 4.14 (s, 3H). ¹⁹F NMR (300 MHz) CDCl₃ δ 62.80. MS: 340.1 (M+1)

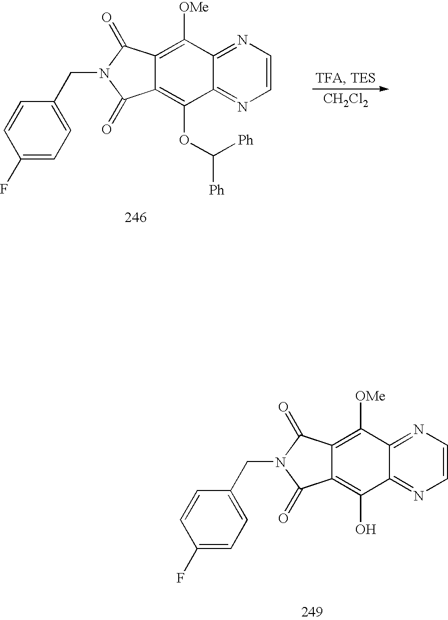

### Example 249

Into a flask containing 5-benzhydryloxy-7-(4-fluoro-benzyl)-9-methoxy-pyrrolo[3,4-g]quinoxaline-6,8-dione 246 (10 mg, 0.019 mmol, 1 equiv.) was added CH₂Cl₂ (1 mL) and under a nitrogen atmosphere was added triethylsilane (200 μL) followed by trifluoroacetic acid (200 μL). The reaction was allowed to stir for 1.5 hours and concentrated in vacuo until thoroughly dried. To the oil was added Hexanes/Ethyl ether (20 mL, 1/1 ratio) and sonicated. The resulting solid was filtered, washed in hexanes and air dried to give 7-(4-fluoro-benzyl)-5-hydroxy-9-methoxy-pyrrolo[3,4-g]quinoxaline-6,8-dione 249 (67%, 4.6 mg, 0.015 mmol). ¹H NMR (300 MHz) CDCl₃ δ 9.07 (d, J=1.8 Hz, 1H), 8.97 (d, J=1.8 Hz, 1H), 7.23-7.27 (m, 2H), 6.96-7.05 (s, 2H), 4.87 (s, 2H), 4.46 (s, 3H). ¹⁹F NMR (300 MHz) CDCl₃ δ 62.77 MS: 354.0 (M+1)

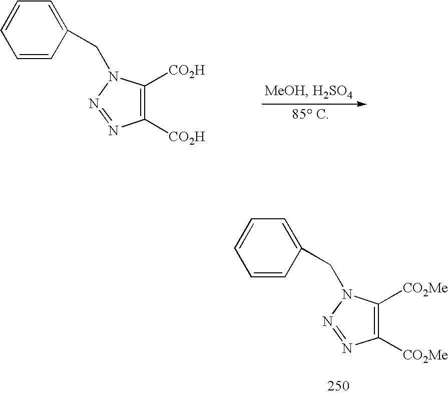

### Example 250

To commercially available, 1-benzyl-1H-[1,2,3]triazole-4,5-dicarboxylic acid (4.5 g, 18.2 mmol, 1 equiv.) was added MeOH (30 mL) followed by dropwise addition of H₂SO₄ (5.5 mL, 103.75 mmol, 5.7 equiv.) over 20 minutes by a method similar to J. Am. Chem. Soc., 73, 1951, 5614-5616. The reaction was heated at 85° C. for 2 h. The reaction was cooled and quenched with water (100 mL). It was extracted with ethyl acetate (4×40 mL) and the organic layer washed several times with water (3×50 mL), saturated NaHCO₃ (50 mL), brine solution (50 mL). It was dried over Na₂SO₄, filtered and concentrated in vacuo to yield 1-Benzyl-1H-[1,2,3]triazole-4,5-dicarboxylic acid dimethyl ester 250 as a brown solid (76%, 3.85 g, 55.9 mmol). ¹H NMR (300 MHz) CDCl₃ δ 7.15-7.33 (m, 5H), 5.41 (s, 2H), 3.92 (s, 3H), 3.84 (s, 3H).

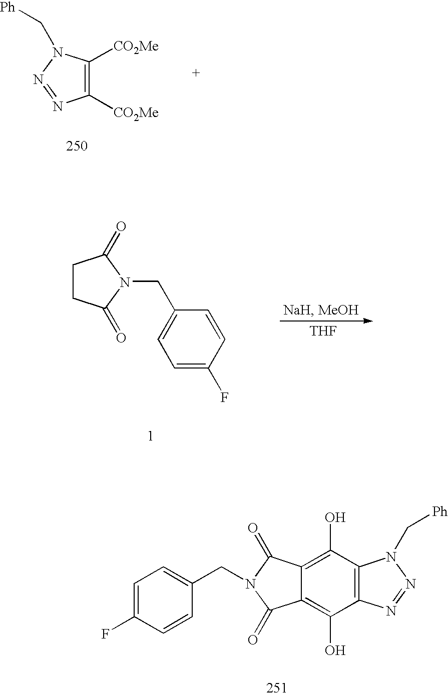

### Example 251

Into a flask containing 1-benzyl-1H-[1,2,3]triazole-4,5-dicarboxylic acid dimethyl ester 250 (3.75 g, 13.64 mmol, 1 equiv.) was added THF (150 mL) under a nitrogen followed by 1-(4-fluoro-benzyl)-pyrrolidine-2,5-dione 1 (2.82 g, 13.64 mmol, 1 equiv.). Methanol (MeOH, 1.1 mL) was added and at 0° C. was added NaH (1.20 g, 29.99 mmol, 2.2 equiv., 60% dispersion) carefully in four portions. Refluxing was carried out for 20 hours after which the reaction was cooled and placed in a 0° C. icebath. HCl (6 N, 20 mL, H₂O) was slowly added while vigorously stirring. The resulting solid was filtered, and washed thoroughly with water followed by ether. It was then dried in a vacuum oven (60° C., overnight) to realize 3.34 gm (60%, 8.18 mmol) of 1-benzyl-6-(4-fluoro-benzyl)-4,8-dihydroxy-1H-pyrrolo[3′,4′:4,5]benzo[1,2-d][1,2,3]triazole-5,7-dione 251. ¹H NMR (300 MHz) CD₃OD δ 9.51 (b, 1H), 7.45-7.35 (m, 8H), 7.15-7.33 (m, 2H), 5.92 (s, 2H), 4.78 (s, 2H).

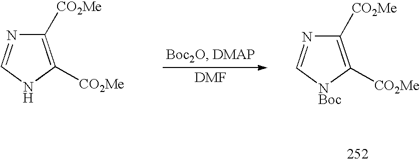

### Example 252

1H-Imidazole-4,5-dicarboxylic acid dimethyl ester (2 g, 10.87 mmol, 1 equiv.) was dissolved in THF (55 mL, 0.2 M) and DMAP (1.46 g, 11.95 mmol, 1.1 equiv.) before Di-tert-butyl dicarbonate (3.50 g, 16.29 mmol, 1.4 equiv.) was added. The reaction was stirred for 16 hours before being quenched with saturated NH₄Cl (30 mL) and extracted with ethyl acetate (2×30 mL) and the organic layer washed several times with water (4×30 mL), brine solution (50 mL). It was dried over Na₂SO₄, filtered and concentrated in vacuo. Imidazole-1,4,5-tricarboxylic acid 1-tert-butyl ester 4,5-dimethyl ester 252 (3.85 g, 100%, 10.87 mmol). ¹H NMR (300 MHz) CDCl₃ δ 8.02 (s, 1H), 3.99 (s, 3H), 3.92 (s, 3H). MS: 306.8 (M+23). TLC R_{f}: 0.6 Hexanes/Ethyl acetate (1/1)

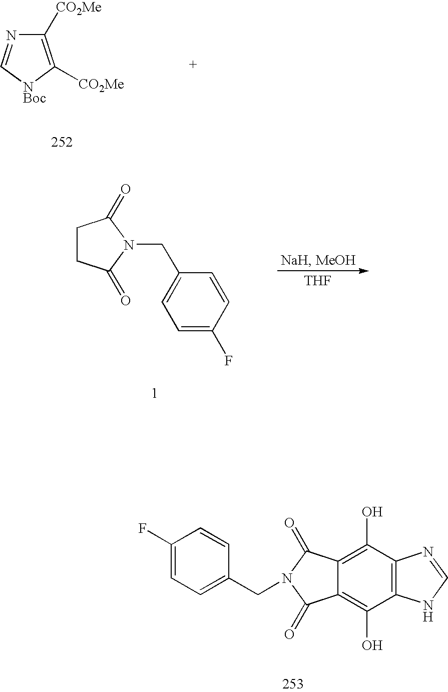

### Example 253

Into a flask containing imidazole-1,4,5-tricarboxylic acid 1-tert-butyl ester 4,5-dimethyl ester 252 (3.85 g, 13.55 mmol, 1 equiv.) was added THF (55 mL) under a nitrogen atmosphere followed by 1-(4-fluoro-benzyl)-pyrrolidine-2,5-dione 1 (2.80 g, 13.55 mmol, 1 equiv.). MeOH (0.4 mL) was added and at 0° C. was added NaH (1.20 g, 29.81 mmol, 2.2 equiv., 60% dispersion) carefully in four portions. Refluxing was carried out for 20 hours after which the reaction was cooled and placed in a 0° C. icebath. HCl (6 N, 30 mL, H₂O) was slowly added while vigorously stirring. The resulting solid was filtered, and washed thoroughly with water followed by ether. It was then dried in a vacuum oven (60° C., overnight) to realize 2.70 gm of a crude solid which was recrystallized with dioxane (650 mL). 6-(4-fluoro-benzyl)-4,8-dihydroxy-1H-1,3,6-triaza-s-indacene-5,7-dione 253 1.65 g, 5.01 mmol). ¹H NMR (300 MHz) DMSO d₆ δ 8.64 (s, 1H), 7.25-7.35 (m, 2H), 7.10-7.29 (m, 2H), 4.66 (s, 2H). ¹⁹F NMR (300 MHz) CDCl₃ δ 61.34. MS: 328.1 (M+1)

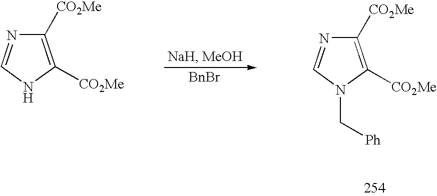

### Example 254

1H-Imidazole-4,5-dicarboxylic acid dimethyl ester (1.5 g, 8.15 mmol, 1 equiv.) was dissolved in MeOH (10 mL) and benzyl bromide (1.16 mL, 9.77 mmol, 1.1 equiv.) before sodium hydride (360 mg, 1.1 equiv., 60% dispersion) and sodium iodide (200 mg) was added. The reaction was stirred for 16 hours before being quenched with saturated NH₄Cl (30 mL) and extracted with ethyl acetate (2×30 mL) and the organic layer washed several times with water (4×30 mL), brine solution (50 mL). It was dried over Na₂SO₄, filtered and concentrated in vacuo. I-Benzyl-1H-imidazole-4,5-dicarboxylic acid dimethyl ester 254 (2.01 g, 90%, 7.33 mmol). ¹H NMR (300 MHz) CDCl₃ δ 7.58 (s, 1H), 7.33-7.42 (m, 3H), 7.14-7.18 (m, 2H), 5.41 (s, 2H), 3.92 (s, 3H), 3.84 (s, 3H). MS: 275.1 (M+1)

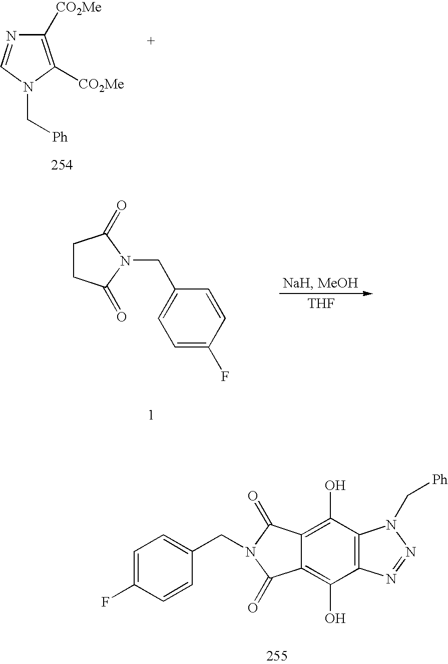

### Example 255

Into a flask containing 1-benzyl-1H-imidazole-4,5-dicarboxylic acid dimethyl ester 254 (2.80 g, 10.22 mmol, 1 equiv.) was added THF (35 mL) under a nitrogen atmosphere followed by 1-(4-Fluoro-benzyl)-pyrrolidine-2,5-dione 1 (2.2 g, 10.22 mmol, 1 equiv.). MeOH (0.5 mL) was then added and at 0° C. was added NaH (940 mg, 23.49 mmol, 2.2 equiv.) carefully in four portions. Refluxing was carried out for 20 hours after which the reaction was cooled and placed in a 0° C. icebath. HCl (6 N, 30 mL, H₂O) was slowly added while vigorously stirring. The resulting solid was filtered, and washed thoroughly with water followed by ether. It was then dried in a vacuum oven (60° C., 12 hours) to realize 4.20 gm of a crude solid. It was recrystallized with dioxane (700 ml) to realize 1-benzyl-6-(4-fluoro-benzyl)-4,8-dihydroxy-1H-1,3,6-triaza-s-indacene-5,7-dione 255 (1.74 g, 41%, 4.19 mmol). ¹H NMR (300 MHz) DMSO d₆ δ 10.40 (bs, 1H), 8.73 (s, 1H), 7.22-7.7.43 (m, 3H), 7.05-7.18 (m, 2H), 5.65 (s, 2H), 4.60 (s, 2H). MS: 418.1 (M+1).

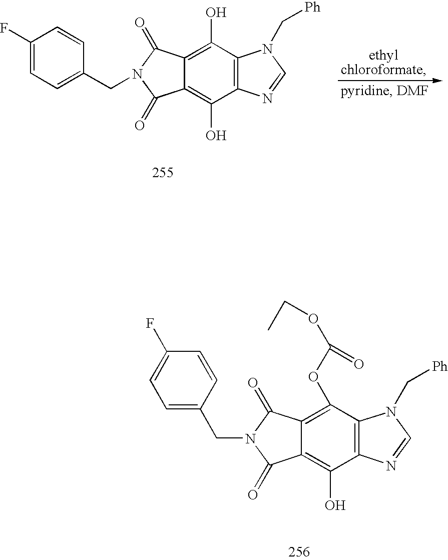

### Example 256

1-Benzyl-6-(4-fluoro-benzyl)-4,8-dihydroxy-1H-1,3,6-triaza-s-indacene-5,7-dione 255 (1 g, 2.39 mmol, 1 equiv.) was dissolved in a flask containing DMF (24 mL, 0.1 M) and pyridine (290 μL, 2.88 mmol, 1.5 equiv.). Ethyl chloroformate was added (231 μL, 2.88 mmol, 1.2 equiv.) under a nitrogen atmosphere. The reaction was stirred for 16 hours before being quenched with saturated NH₄Cl (30 mL) and extracted with ethyl acetate (2×30 mL) and the organic layer washed several times with water (4×30 mL), saturated NaHCO₃ (50 mL), brine solution (50 mL). It was dried over Na₂SO₄, filtered and concentrated in vacuo. Trituration was carried out with Hexanes/Ethyl acetate (1/4, 100 mL) to remove the corresponding biscarbonate to give carbonic acid 3-benzyl-6-(4-fluoro-benzyl)-8-hydroxy-5,7-dioxo-3,5,6,7-tetrahydro-1,3,6-triaza-s-indacen-4-yl ester ethyl ester 256 (13%, 145 mg, 0.296 mmol). ¹H NMR (300 MHz) DMSO d₆ δ 8.63 (s, 1H), 7.45-7.35 (m, 6H), 7.15-7.33 (m, 4H), 5.59 (s, 2H), 4.63 (s, 2H), 3.98 (q, J=6.9 Hz, 2H), 1.17 (t, J=6.9 Hz, 3H). MS: 490.2 (M+1). TLC R_{f}: 0.6 Ethyl acetate.

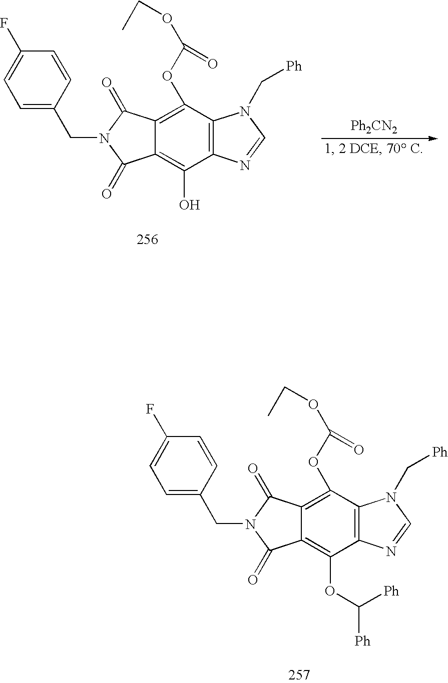

### Example 257

Carbonic acid 3-benzyl-6-(4-fluoro-benzyl)-8-hydroxy-5,7-dioxo-3,5,6,7-tetrahydro-1,3,6-triaza-s-indacen-4-yl ester ethyl ester 256 (140 mg, 0.28 mmol, 1 equiv.) was dissolved in 1,2 dichloroethane (20 mL) and to this was added diphenyldiazomethane (72 mg, 0.37 mmol, 1.3 equiv.) and heated at 70° C. under a nitrogen atmosphere for 24 hours. The reaction was then concentrated in vacuo and purified by silica gel chromatography using 7/3 Hexanes/Ethyl acetate to obtain carbonic acid 8-benzhydryloxy-3-benzyl-6-(4-fluoro-benzyl)-5,7-dioxo-3,5,6,7-tetrahydro-1,3,6-triaza-s-indacen-4-yl ester ethyl ester 257 (78%, 135 mg, 0.22 mmol). ¹H NMR (300 MHz) CDCl₃ δ 8.17 (s, 1H), 7.91 (s, 1H), 7.68 (d, J=7.2 Hz, 4H), 7.21-7.42 (m, 12H), 6.95-7.06 (s, 4H), 5.49 (s, 2H), 4.76 (s, 2H), 4.11 (q, J=6.9 Hz, 2H), 1.17 (t, J=6.9 Hz, 3H). MS: 678.1 (M+23). TLC R_{f}: 0.3 Hexanes/Ethyl acetate (7/3)

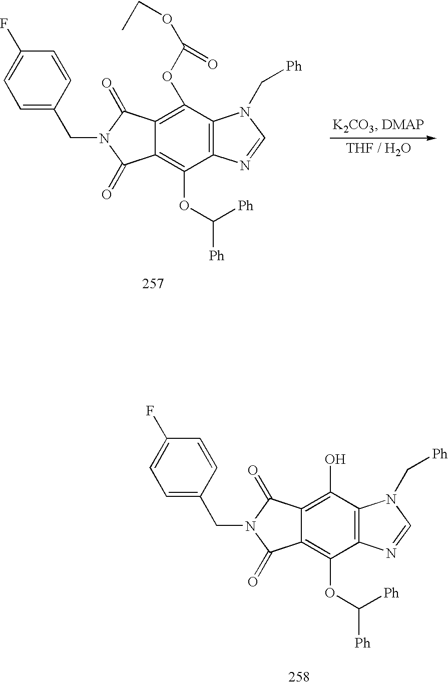

### Example 258

Carbonic acid 8-benzhydryloxy-3-benzyl-6-(4-fluoro-benzyl)-5,7-dioxo-3,5,6,7-tetrahydro-1,3,6-triaza-s-indacen-4-yl ester ethyl ester 257 (130 mg, 0.20 mmol) was dissolved in THF (5 mL, 0.1 M) along with DMAP (24 mg, 0.40 mmol, 2 equiv.). A solution of K₂CO₃ (276 mg, 1.99 mmol, 10 equiv.) was dissolved separately in H₂O (6 mL) before transferring to the reaction mixture. The reaction was allowed to stir for 18 hr and quenched with HCl (20 mL, 1 N) and extracted with ethyl acetate (2×30 ml). The organic layer was washed with saturated NH₄Cl solution (25 mL), brine solution (25 mL) and dried over Na₂SO₄ and concentrated in vacuo to yield 4-Benzhydryloxy-1-benzyl-6-(4-fluoro-benzyl)-8-hydroxy-1H-1,3,6-triaza-s-indacene-5,7-dione 258 (94%, 103 mg, 0.188 mmol) as an off white oil. ¹H NMR (300 MHz) CDCl₃ δ 8.28 (bs, 1H), 7.94 (s, 1H), 7.89 (s, 1H), 7.64-7.43 (m, 4H), 7.17-7.43 (m, 12H), 6.98-7.04 (s, 2H), 5.57 (s, 2H), 4.77 (s, 2H). MS: 584.1 (M+1).

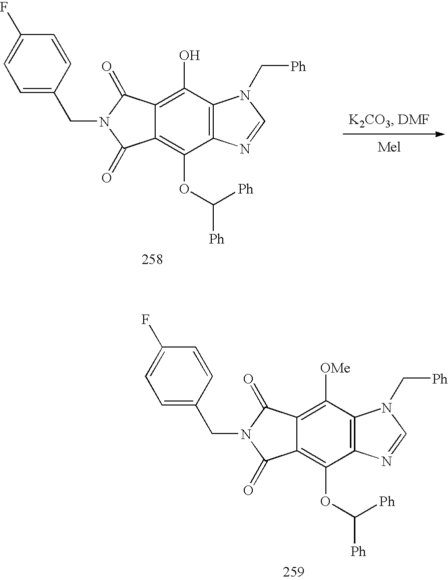

### Example 259

Benzhydryloxy-1-benzyl-6-(4-fluoro-benzyl)-8-hydroxy-1H-1,3,6-triaza-s-indacene-5,7-dione 258 (103 mg, 0.177 mmol, 1 equiv.) was added to a flask containing DMF (4 mL) followed by K₂CO₃ (122 mg, 0.88 mmol, 5 equiv.). To this was added methyl iodide (MeI, 109 μL, 1.76 mmol, 10 equiv.) under a nitrogen atmosphere and stirred for 16 hours. To the reaction was added water (50 mL) and extracted with ethyl acetate (2×40 mL). The organic layer was washed several times with water (3×30 mL), saturated NaHCO₃ (40 mL), brine solution (30 mL). It was dried over Na₂SO₄, filtered and concentrated in vacuo and purified by silica gel chromatography using 7/3 Hexanes/Ethyl acetate to obtain 4-benzhydryloxy-1-benzyl-6-(4-fluoro-benzyl)-8-methoxy-1H-1,3,6-triaza-s-indacene-5,7-dione 259 (73%, 75 mg, 0.125 mmol). ¹H NMR (300 MHz) CDCl₃ δ 8.09 (s, 1H), 7.94 (s, 1H), 7.88 (s, 1H), 7.64-7.43 (m, 4H), 7.41-7.46 (m, 2H), 7.17-7.43 (m, 10H), 6.98-7.04 (m, 3H), 5.56 (s, 2H), 4.80 (s, 2H), 3.84 (s, 3H). MS: 620.1 (M+23). TLC R_{f}: 0.6 Hexanes/Ethyl acetate (1/1).

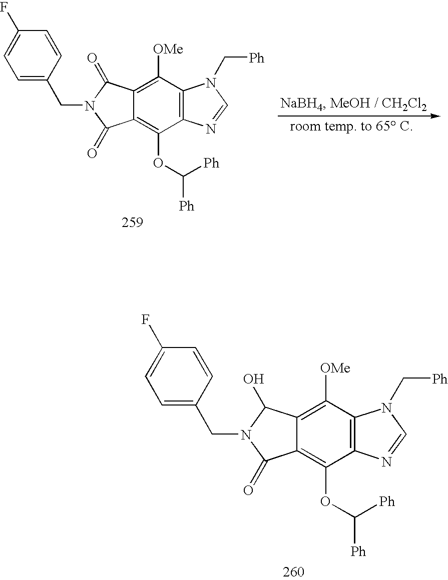

### Example 260

4-Benzhydryloxy-1-benzyl-6-(4-fluoro-benzyl)-8-methoxy-1H-1,3,6-triaza-s-indacene-5,7-dione 259 (54 mg, 0.092 mmol, 1 equiv.) was dissolved in CH₂Cl₂ (2 mL) and MeOH (0.5 mL) and under a nitrogen atmosphere. Sodium borohydride (NaBH₄, 736 μL, 0.37 mmol, 4 equiv., 0.5 M) was added. The reaction was allowed to stir for 1 hour at room temperature and heated to 65° C. for 2 hours before being quenched with water (5 mL) and extracted with ethyl acetate (2×5 mL). The organic layer was washed several times with water (2×10 mL), brine solution (10 mL). It was dried over Na₂SO₄, filtered and concentrated in vacuo and purified by preparatory thin-layer chromatography (PTLC) using 3/2 Hexanes/Ethyl acetate to obtain 260 (51%, 28 mg, 0.047 mmol).

¹H NMR (300 MHz) CDCl₃ δ 7.86 (d, J=7.2 Hz, 2H), 7.59 (d, J=7.2 Hz, 2H), 7.46-7.32 (m, 4H), 7.32-7.21 (m, 4H), 7.03-7.18 (m, 6H), 6.91-7.01 (m, 2H), 5.95 (bs, 1H), 5.56 (s, 2H), 5.62-5.52 (m, 1H), 5.28 (d, J=15.9 Hz, 1H), 5.14 (d, J=15.9 Hz, 1H), 4.49 (d, J=15.9 Hz, 1H), 3.37 (s, 3H). MS: 622.0 (M+23). TLC R_{f}: 0.25 Hexanes/Ethyl acetate (3/2)

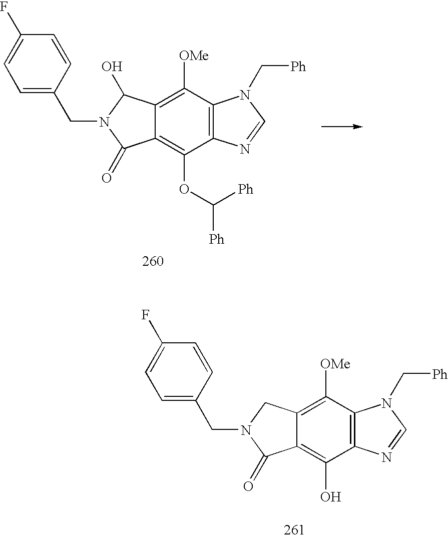

### Example 261

4-Benzhydryloxy-1-benzyl-6-(4-fluoro-benzyl)-7-hydroxy-8-methoxy-6,7-dihydro-1H-1,3,6-triaza-s-indacen-5-one 260 (28 mg, 0.047 mmol, 1 equiv.) was added to CH₂Cl₂ (1 mL) under a nitrogen atmosphere. Triethylsilane (200 μL) was added, followed by trifluoroacetic acid (200 μL). The reaction was allowed 1 hour and concentrated in vacuo until thoroughly dried. Hexanes/Ethyl ether (15 mL, 1/1 ratio) was added to the oil and sonicated. The resulting solid was then filtered and washed in Hexanes and air dried to give 7-(4-fluoro-benzyl)-5-hydroxy-9-methoxy-7,8-dihydro-pyrrolo[3,4-g]quinoxalin-6-one 261 (100%, 20 mg, 0.047 mmol) as a light gray powder.

¹H NMR (300 MHz) CDCl₃ δ 9.11 (bs 1H), 7.86 (s, 1H), 7.33-7.23 (m, 5H), 7.01 7.07 (s, 4H), 5.57 (s, 2H), 4.71 (s, 2H), 4.37 (s, 2H), 3.57 (s, 3H). ¹⁹F NMR (300 MHz) CDCl₃ δ 62.25. MS: 418.2 (M+1)

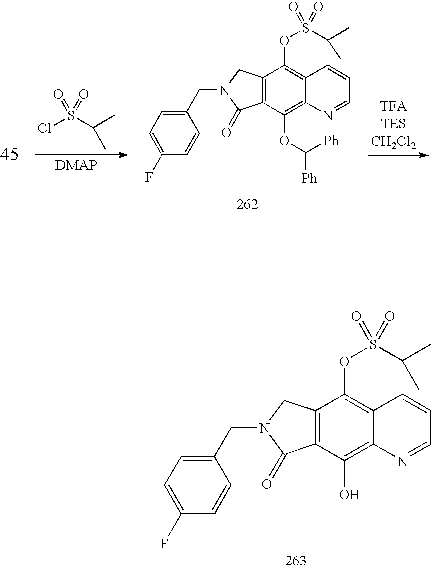

### Example 262

To 0.051 mmol crude 45 was added triethylamine (100 μl), DMAP (catalytic amount) and isopropylsulfonyl chloride (18 μl, 0.154 mmol). The reaction mixture was stirred at room temperature for 24 hours under an inert atmosphere. The reaction was monitored by TLC (EtOAc/hexane 3/7) (R_{f}44=0.5, R_{f}45=0, R_{f}262=0.2) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—3/7) to afford propane-2-sulfonic acid 9-benzhydryloxy-7-(4-fluoro-benzyl)-8-oxo-7,8-dihydro-6H-pyrrolo[3,4-g]quinolin-5-yl ester 262 (8.7 mg, 29%).

### Example 263

To a solution of 262 (8.7 mg, 0.015 mmol) dissolved in dichloromethane (1 mL) was added trifluoroacetic acid (100 μl) and triethylsilane (200 μl). The reaction mixture was stirred at room temperature for 30 min under an inert atmosphere then concentrated in vacuo. The residue was triturated with diethyl ether/hexane (1/1) to afford the trifluoroacetate salt of propane-2-sulfonic acid 7-(4-fluoro-benzyl)-9-hydroxy-8-oxo-7,8-dihydro-6H-pyrrolo[3,4-g]quinolin-5-yl ester 263 (5.3 mg, 0.010 mmol, 68%) as a yellow solid: ¹H NMR (CDCl₃) δ 9.0 (d, 1H), 8.4 (d, 1H), 7.6 (m, 1H), 7.3 (m, 2H), 7.0 (t, 2H), 4.8 (s, 2H), 4.6 (s, 2H), 3.7 (m, 1H), 1.7 (m, 6H); MS: 431 (M+1).

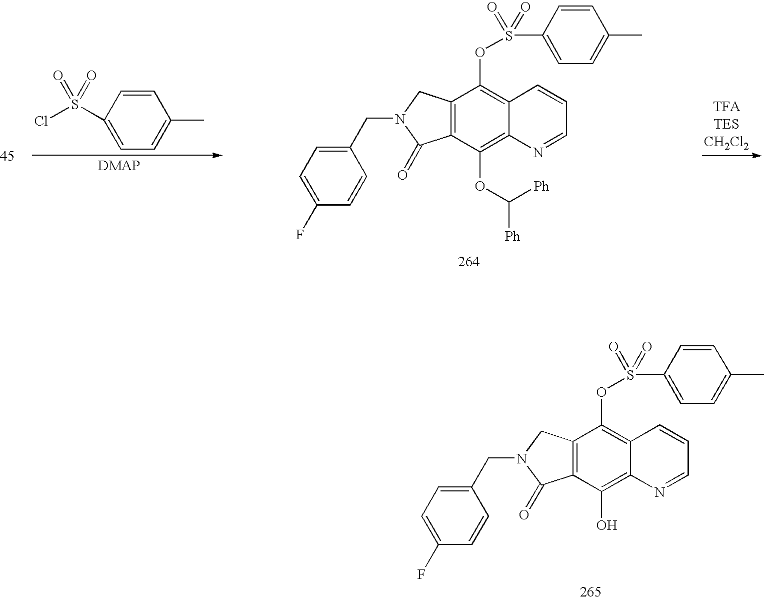

### Example 264

Triethylamine (100 μl), DMAP (catalytic amount) and p-tosyl-chloride (30 mg, 0.154 mmol) were added to 0.051 mmol 45. The reaction mixture was stirred at room temperature for 24 hours under an inert atmosphere. The reaction was monitored by TLC (EtOAc/hexane 3/7) (R_{f}44=0.5, R_{f}45=0, R_{f}264=0.3) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—3/7) to afford toluene-4-sulfonic acid 9-benzhydryloxy-7-(4-fluoro-benzyl)-8-oxo-7,8-dihydro-6H-pyrrolo[3,4-g]quinolin-5-yl ester 264 (15.3 mg, 47%).

### Example 265

To a solution of 264 (15.3 mg, 0.015 mmol) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford the trifluoroacetate salt of toluene-4-sulfonic acid 7-(4-fluoro-benzyl)-9-hydroxy-8-oxo-7,8-dihydro-6H-pyrrolo[3,4-g]quinolin-5-yl ester 265 (11.6 mg, 0.020 mmol, 83%) as a yellow solid: ¹H NMR (CDCl₃) δ 8.9 (d, 1H), 8.0 (d, 1H), 7.8 (m, 1H), 7.3 (m, 6H), 7.0 (t, 2H), 5.3 (s, 1H, OH), 4.7 (s, 2H), 4.4 (s, 2H), 2.4 (s, 3H); MS: 479 (M+1).

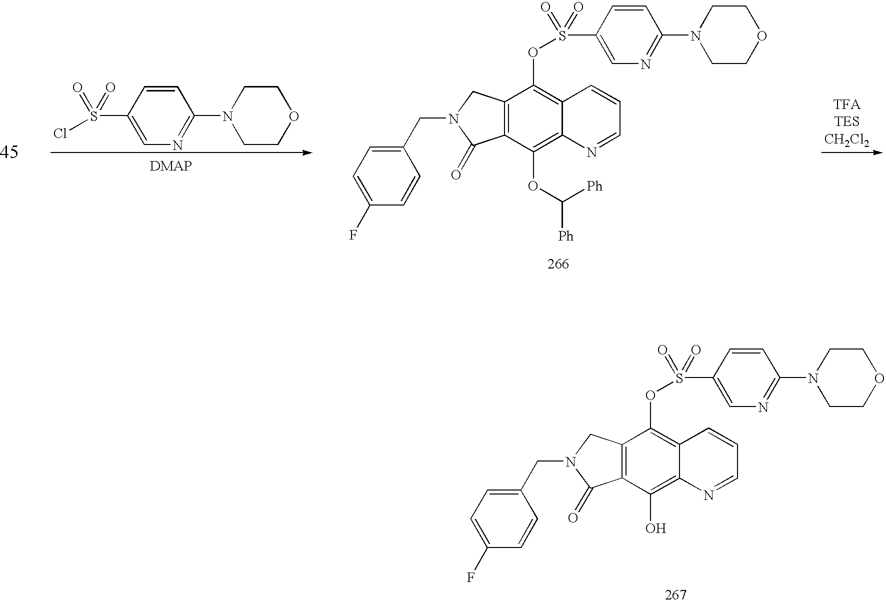

### Example 266

Triethylamine (50 μl), DMAP (catalytic amount) and 6-Morpholin-4-yl-pyridine-3-sulfonyl chloride (26.3 mg, 0.10 mmol) were added to 0.034 mmol 45. The reaction mixture was stirred at room temperature for 18 hours under an inert atmosphere. The reaction was monitored by TLC (EtOAc/hexane 3/7) (R_{f}44=0.5, R_{f} 45=0, R_{f}266=0.3) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—3/7) to afford 6-morpholin-4-yl-pyridine-3-sulfonic acid 9-benzhydryloxy-7-(4-fluoro-benzyl)-8-oxo-7,8-dihydro-6H-pyrrolo[3,4-g]quinolin-5-yl ester 266 (14.6 mg, 59%).

### Example 267

To a solution of 266 (14.6 mg, 0.020 mmol) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford the TFA salt of 6-morpholin-4-yl-pyridine-3-sulfonic acid 7-(4-fluoro-benzyl)-9-hydroxy-8-oxo-7,8-dihydro-6H-pyrrolo[3,4-g]quinolin-5-yl ester 267 (9.0 mg, 68%) as a yellow solid: ¹H NMR (CDCl₃) δ 8.9 (d, 1H), 8.6 (s, 1H), 8.0 (dd, 1H), 7.7 (dd, 1H), 7.5 (m, 1H), 7.3 (m, 2H), 7.0 (t, 2H), 6.5 (d, 2H), 4.8 (s, 2H), 4.6 (s, 2H), 3.7 (d, 4H), 3.6 (d, 4H); MS: 551 (M+1).

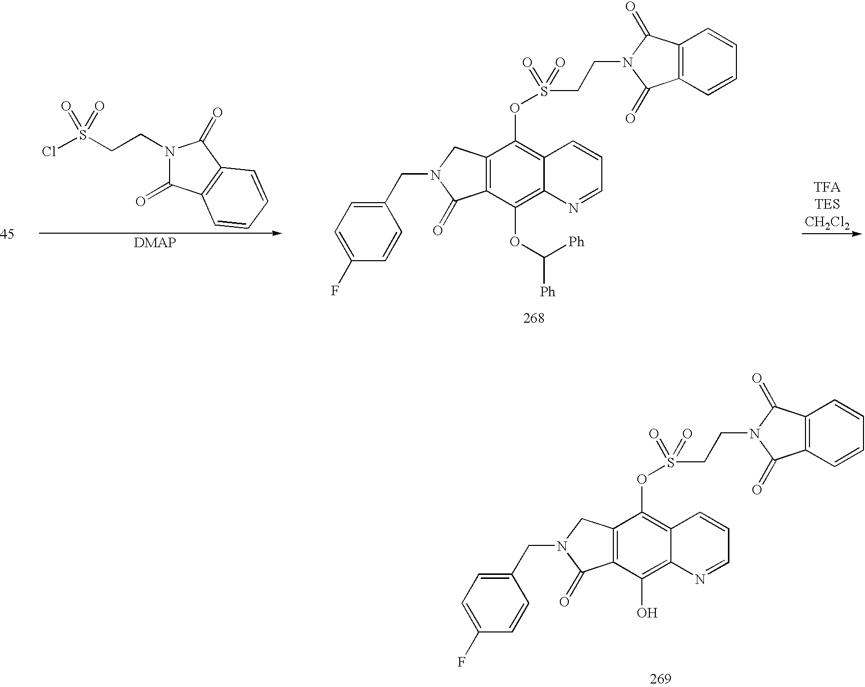

### Example 268

Triethylamine (50 μl), DMAP (catalytic amount) and 2-(1,3-dioxo-1,3-dihydro-isoindol-2-yl)-ethanesulfonyl chloride (27.4 mg, 0.10 mmol) were added to 0.034 mmol 45. The reaction mixture was stirred at room temperature for 18 hours under an inert atmosphere. The reaction was monitored by TLC (EtOAc/hexane 3/7) (R_{f}44=0.5, R_{f}45=0, R_{f}268=0.4) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—3/7) to afford 2-(1,3-dioxo-1,3-dihydro-isoindol-2-yl)-ethanesulfonic acid 9-benzhydryloxy-7-(4-fluoro-benzyl)-8-oxo-7,8-dihydro-6H-pyrrolo[3,4-g]quinolin-5-yl ester 268 (12.2 mg, 50%).

### Example 269

To a solution of 268 (12.2 mg, 0.017 mmol) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford 2-(1,3-dioxo-1,3-dihydro-isoindol-2-yl)-ethanesulfonic acid 7-(4-fluoro-benzyl)-9-hydroxy-8-oxo-7,8-dihydro-6H-pyrrolo[3,4-g]quinolin-5-yl ester 269, TFA salt, (9.0 mg, 76%) as a yellow solid: ¹H NMR (CDCl₃) δ 9.0 (d, 1H), 8.5 (dd, 1H), 7.9 (m, 2H), 7.8 (m, 2H), 7.7 (m, 1H), 7.3 (m, 2H), 7.0 (t, 2H), 4.8 (s, 2H), 4.6 (s, 2H), 4.4 (q, 2H), 3.9 (q, 2H); MS: 562 (M+1).

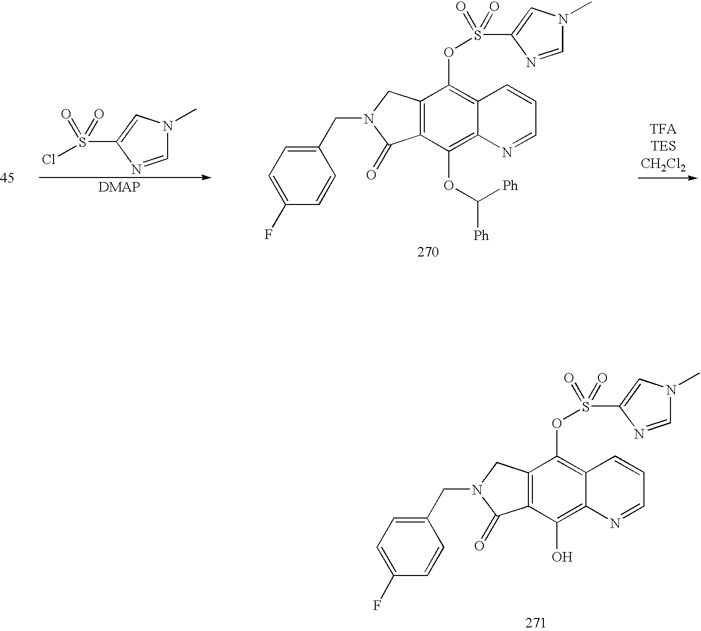

### Example 270

Triethylamine (50 μl), DMAP (catalytic amount) and 1-methyl-1H-imidazole-4-sulfonyl chloride (18.1 mg, 0.10 mmol) were added to 0.034 mmol crude 45. The reaction mixture was stirred at room temperature for 18 hours under an inert atmosphere. The reaction was monitored by TLC (EtOAc/hexane 3/7) (R_{f}44=0.5, R_{f} 45=0, R_{f}270=0.05) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo to give the crude mixture of 1-methyl-1H-imidazole-4-sulfonic acid 9-benzhydryloxy-7-(4-fluoro-benzyl)-8-oxo-7,8-dihydro-6H-pyrrolo[3,4-g]quinolin-5-yl ester 270.

### Example 271

To a solution of crude 270 dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was purified by HPLC to afford 1-methyl-1H-imidazole-4-sulfonic acid 7-(4-fluoro-benzyl)-9-hydroxy-8-oxo-7,8-dihydro-6H-pyrrolo[3,4-g]quinolin-5-yl ester 271, TFA salt, (2.5 mg) as a yellow solid: ¹H NMR (CD₃OD) δ 8.9 (d, 1H), 8.4 (d, 1H), 7.85 (s, 1H), 7.78 (s, 1H), 7.6 (m, 1H), 7.4 (m, 2H), 7.1 (t, 2H), 4.8 (s, 2H), 4.5 (s, 2H), 3.8 (s, 3H); MS: 469 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18(2), 150 mm×21.1 mm.

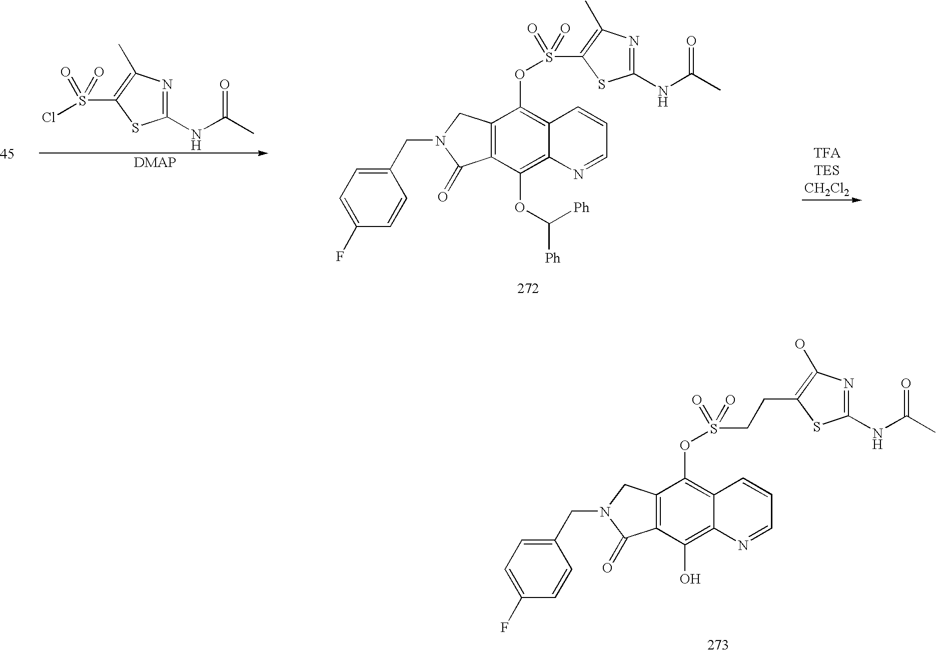

### Example 272

Triethylamine (50 μl), DMAP (catalytic amount) and 2-acetylamino-4-methyl-thiazole-5-sulfonyl chloride (25.5 mg, 0.10 mmol) were added to 0.034 mmol 45. The reaction mixture was stirred at room temperature for 18 hours under an inert atmosphere. The reaction was monitored by TLC (EtOAc/hexane 3/7) (R_{f}44=0.5, R_{f} 45=0, R_{f}272=0.2) and LC/MS. After completion of the reaction, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—3/7) to afford 2-acetylamino-4-methyl-thiazole-5-sulfonic acid 9-benzhydryloxy-7-(4-fluoro-benzyl)-8-oxo-7,8-dihydro-6H-pyrrolo[3,4-g]quinolin-5-yl ester 272 (18.9 mg, 79%).

### Example 273

To a solution of 272 (18.9 mg, 0.027 mmol) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford 2-acetylamino-4-methyl-thiazole-5-sulfonic acid 7-(4-fluoro-benzyl)-9-hydroxy-8-oxo-7,8-dihydro-6H-pyrrolo[3,4-g]quinolin-5-yl ester 273, TFA salt, (13.2 mg, 74%) as a yellow solid: ¹H NMR (CD₃OD) δ 8.9 (d, 1H), 8.2 (d, 1H), 7.6 (m, 1H), 7.4 (m, 2H), 7.1 (t, 2H), 4.7 (s, 2H), 4.4 (s, 2H), 2.23 (s, 3H), 2.21 (s, 3H); MS: 543 (M+1).

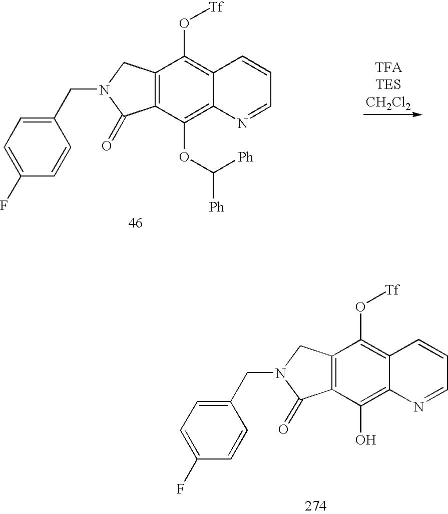

### Example 274

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (28 mg, 0.045 mmol) dissolved in dichloromethane (2 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford trifluoro-methanesulfonic acid 7-(4-fluoro-benzyl)-9-hydroxy-8-oxo-7,8-dihydro-6H-pyrrolo[3,4-g]quinolin-5-yl ester 274, (13.7 mg, 0.03 mmol, 67%) as a yellow solid: ¹H NMR (CDCl₃) δ 9.0 (d, 1H), 8.4 (d, 1H), 7.7 (dd, 1H), 7.3 (dd, 2H), 7.1 (t, 2H), 4.8 (s, 2H), 4.6 (s, 2H); MS: 457 (M+1).

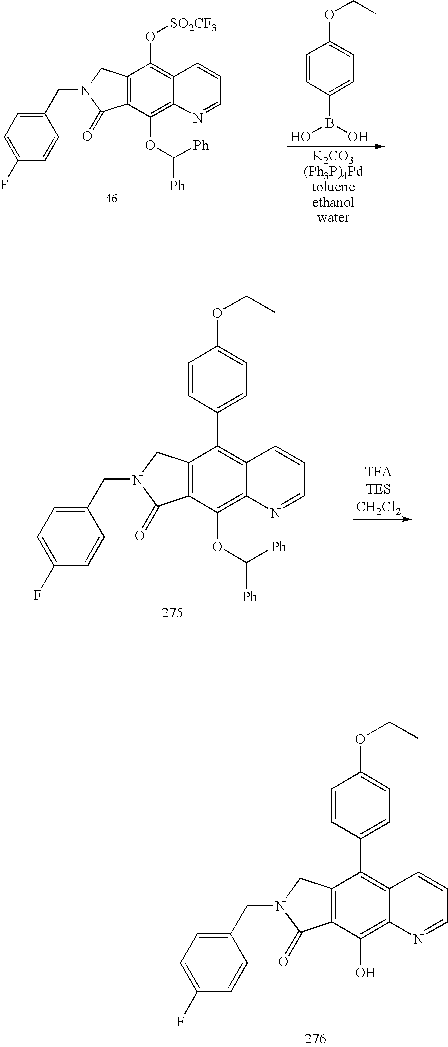

### Example 275

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (40 mg, 0.064 mmol) dissolved in toluene (3 mL)/ethanol (0.6 mL)/water (0.4 mL) was added K₂CO₃ (27 mg, 0.192 mmol), 4-ethoxyphenolboronic acid (22 mg, 0.128 mmol) and tetrakis-(triphenylphosphine)-palladium(0) (15 mg, 0.013 mmol). The reaction mixture in the flask was flashed with argon three times. It was then heated to 120° C. under argon 3 hours. The reaction was monitored by TLC (EtOAc/hexane 3/7) (R_{f}46=0.6, R_{f}275=0.4) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by silica gel chromatography (ethylacetate/hexane—1/3) to afford 9-benzhydryloxy-5-(4-ethoxy-phenyl)-7-(4-fluoro-benzyl)-6,7-dihydro-pyrrolo[3,4-g]quinolin-8-one 275 (8.0 mg, 21%) as a solid: ¹H NMR (CDCl₃) δ 9.0 (d, 1H), 8.1 (s, 1H), 7.9 (d, 1H), 7.8-7.5 (dd, 4H), 7.5 (s, 1H), 7.4 (dd, 2H), 7.3-7.1 (m, 10H), 7.0 (t, 2H), 4.8 (s, 2H), 4.1 (m, 2H), 4.0 (s, 1H), 1.4 (t, 3H); MS: 595 (M+1).

### Example 276

To a solution of 9-benzhydryloxy-5-(4-ethoxy-phenyl)-7-(4-fluoro-benzyl)-6,7-dihydro-pyrrolo[3,4-g]quinolin-8-one 275 (8 mg, 0.013 mmol) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford 5-(4-ethoxy-phenyl)-7-(4-fluoro-benzyl)-9-hydroxy-6,7-dihydro-pyrrolo[3,4-g]quinolin-8-one 276, TFA salt, (1.8 mg, 0.003 mmol, 25%) as a yellow solid: ¹H NMR (CDCl₃) δ 9.0 (d, 1H), 8.1 (d, 1H), 7.7 (m, 2H), 7.6 (dd, 1H), 7.5 (dd, 2H), 7.2 (dd, 2H), 7.1 (t, 2H), 4.7 (s, 2H), 4.2 (s, 2H), 4.1 (m, 2H), 1.5 (t, 3H); MS: 429 (M+1).

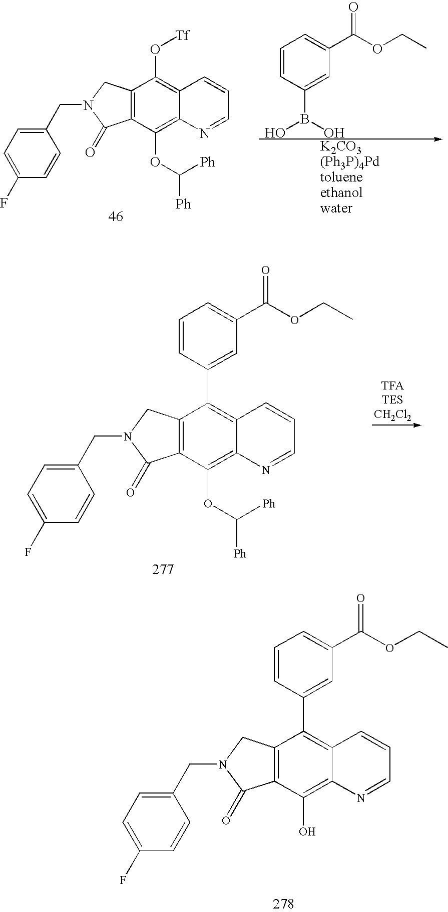

### Example 277

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (43 mg, 0.07 mmol) dissolved in toluene (3 mL)/ethanol (0.6 mL)/water (0.4 mL) was added K₂CO₃ (29 mg, 0.21 mmol), (3-ethoxycarbonylphenyl)boronic acid (28 mg, 0.14 mmol) and tetrakis-(triphenylphosphine)-palladium(0) (16 mg, 0.014 mmol). The reaction mixture in the flask was flashed with argon three times. It was then heated to 120° C. under argon 3 hours. The reaction was monitored by TLC (EtOAc/hexane 3/7) (R_{f}46=0.6, R_{f}277=0.3) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo to afford crude 3-[9-benzhydryloxy-7-(4-fluoro-benzyl)-8-oxo-7,8-dihydro-6H-pyrrolo[3,4-g]quinolin-5-yl]-benzoic acid ethyl ester 277.

### Example 278

To a solution of 277 dissolved in dichloromethane (2 mL) was added trifluoroacetic acid (200 μl) and triethylsilane (400 μl). The reaction mixture was stirred at room temperature for ½ hours under an inert atmosphere then concentrated in vacuo. The residue was redissolved in DMSO (1 mL) and purified by prep-HPLC to afford 3-[7-(4-fluoro-benzyl)-9-hydroxy-8-oxo-7,8-dihydro-6H-pyrrolo[3,4-g]quinolin-5-yl]-benzoic acid ethyl ester 278, TFA salt, (25 mg, 0.003 mmol, 44% in two steps) as a yellow solid: ¹H NMR (CDCl₃) δ 9.0 (d, 1H), 8.2 (d, 1H), 8.0 (s, 1H), 7.7 (m, 1H), 7.6 (dd, 1H), 7.5 (dd, 2H), 7.0 (m, 2H), 7.1 (t, 2H), 4.7 (dd, 2H), 4.4 (q, 2H), 4.3 (dd, 2H), 1.4 (t, 3H); MS: 457 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18 (2), 150 mm×21.1 mm.

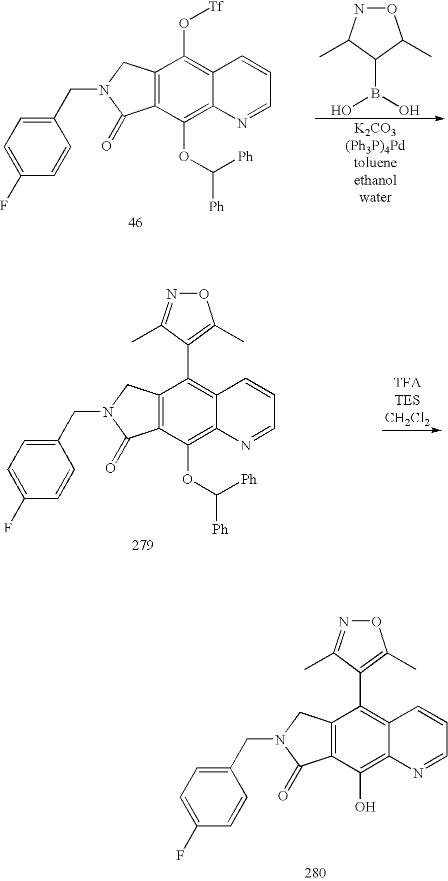

### Example 279

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (23.6 mg, 0.038 mmol) dissolved in toluene (3 mL)/ethanol (0.6 mL)/water (0.4 mL) was added K₂CO₃ (16 mg, 0.11 mmol), 3,5-dimethylisoxazole-4-boronic acid (11 mg, 0.076 mmol) and tetrakis-(triphenylphosphine)-palladium(0) (9 mg, 0.007 mmol). The reaction mixture in the flask was flashed with argon three times. It was then heated to 120° C. under argon 3 hours. The reaction was monitored by LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo to afford crude 9-benzhydryloxy-5-(3,5-dimethyl-isoxazol-4-yl)-7-(4-fluoro-benzyl)-6,7-dihydro-pyrrolo[3,4-g]quinolin-8-one 279.

### Example 280

To a solution of 279 dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was dissolved in DMSO (1 mL) and purified by prep-HPLC to remove Ph₃PO. The crude mixture was diluted with EtOAC and extracted with 1N HCl. The aqueous phase containing product 280 was re-purified by HPLC to afford 5-(3,5-dimethyl-isoxazol-4-yl)-7-(4-fluoro-benzyl)-9-hydroxy-6,7-dihydro-pyrrolo[3,4-g]quinolin-8-one 280, (0.4 mg) as a TFA salt solid: ¹H NMR (CD₃OD) δ 9.0 (d, 1H), 8.1 (d, 1H), 8.0 (s, 1H), 7.7 (m, 1H), 7.4 (dd, 1H), 7.1 (t, 2H), 4.8 (s, 2H), 4.2 (s, 2H), 2.0 (s, s, 2×3H); MS: 404 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18 (2), 150 mm×21.1 mm.

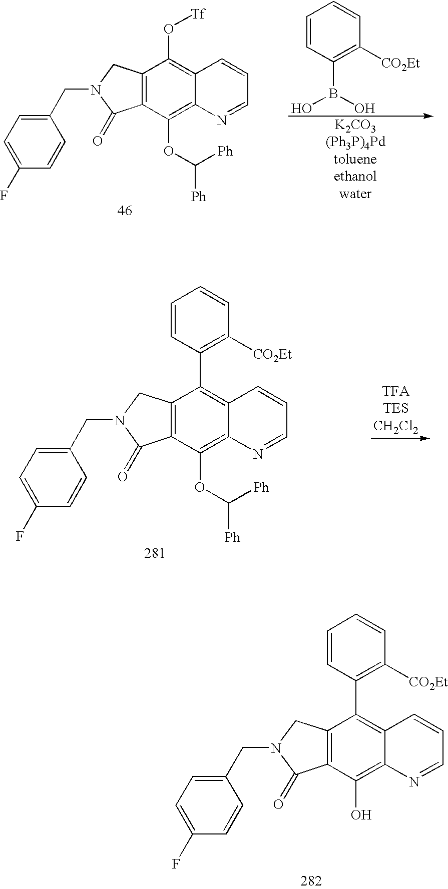

### Example 281

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (33.5 mg, 0.05 mmol) dissolved in toluene (3 mL)/ethanol (0.6 mL)/water (0.4 mL) was added K₂CO₃ (22 mg, 0.15 mmol), (2-ethoxycarbonylphenyl)boronic acid (22 mg, 0.10 mmol) and tetrakis-(triphenylphosphine)-palladium(0) (12.5 mg, 0.01 mmol). The reaction mixture in the flask was flashed with argon three times. It was then heated to 120° C. under argon 3 hours. The reaction was monitored by TLC (EtOAc/hexane 3/7) (R_{f}46=0.6, R_{f}281=0.5) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by flash chromatography on silica gel with EtOAc/Hexane (3/7) to afford pure 2-[9-benzhydryloxy-7-(4-fluoro-benzyl)-8-oxo-7,8-dihydro-6H-pyrrolo[3,4-g]quinolin-5-yl]-benzoic acid ethyl ester 281, 9 mg, 26.8%.

### Example 282

To a solution of 281 dissolved in dichloromethane (2 mL) was added trifluoroacetic acid (200 μl) and triethylsilane (400 μl). The reaction mixture was stirred at room temperature for ½ hours under an inert atmosphere then concentrated in vacuo. The residue was triturated with diethyl ether/hexane (1/1) to afford 2-[7-(4-fluoro-benzyl)-9-hydroxy-8-oxo-7,8-dihydro-6H-pyrrolo[3,4-g]quinolin-5-yl]-benzoic acid ethyl ester 282, TFA salt, (2.5 mg) as a yellow solid: ¹H NMR (CD₃OD) δ 8.9 (d, 1H), 8.0 (d, 1H), 8.0 (s, 1H), 7.8-7.6 (m, 3H), 7.5 (dd, 1H), 7.3 (m, 2H+1H), 7.0 (t, 2H), 4.7 (dd, 2H), 4.1 (dd, 2H), 3.7 (m, 2H), 0.6 (t, 3H); MS: 457 (M+1).

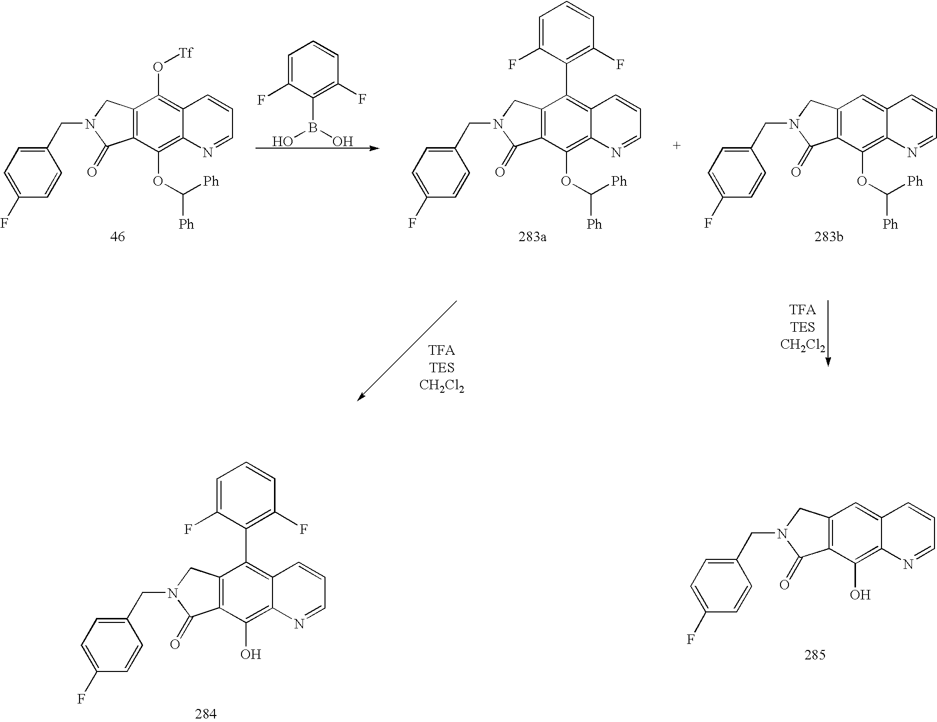

### Example 283

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (40 mg, 0.064 mmol) dissolved in toluene (3 mL)/ethanol (0.6 mL)/water (0.4 mL) was added K₂CO₃ (29 mg, 0.16 mmol), (2,6-difluorophenyl)boronic acid (20 mg, 0.128 mmol) and tetrakis-(triphenylphosphine)-palladium(0) (15 mg, 0.01 mmol). The reaction mixture in the flask was flashed with argon three times. It was then heated to 120° C. under argon for 3 hours. The reaction was monitored by TLC (EtOAc/hexane 3/7) (R_{f} 46=0.6, R_{f}283a=0.4, R_{f}283b=0.3) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by flash chromatography on silica gel with EtOAc/Hexane (3/7) to separate pure 9-benzhydryloxy-5-(2,6-difluoro-phenyl)-7-(4-fluoro-benzyl)-6,7-dihydro-pyrrolo[3,4-g]quinolin-8-one 283a, 6 mg, 17%; and pure 9-benzhydryloxy-7-(4-fluoro-benzyl)-6,7-dihydro-pyrrolo[3,4-g]quinolin-8-one 283b, 11.0 mg, 36%.

### Example 284

To a solution of 283a (9 mg) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford 5-(2,6-difluoro-phenyl)-7-(4-fluoro-benzyl)-9-hydroxy-6,7-dihydro-pyrrolo[3,4-g]quinolin-8-one 284, TFA salt, (3.2 mg) as a yellow solid: ¹H NMR (CDCl₃) δ 9.0 (d, 1H), 8.0 (d, 1H), 7.6 (m, 1H), 7.5 (dd, 1H), 7.2 (m, 2H), 7.1 (m, 4H), 4.7 (s, 2H), 4.2 (s, 2H); MS: 421 (M+1).

### Example 285

To a solution of 283b (1 mg) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was triturated with diethyl ether/hexane (1/1) to afford 7-(4-fluoro-benzyl)-9-hydroxy-6,7-dihydro-pyrrolo[3,4-g]quinolin-8-one 285, TFA salt, (3.9 mg) as a yellow solid: ¹H NMR (CDCl₃) δ 9.1 (d, 1H), 8.3 (d, 1H), 7.6 (m, 1H), 7.35 (s, 1H), 7.33 (m, 2H), 7.0 (t, 2H), 4.8 (s, 2H), 4.4 (s, 2H); MS: 309 (M+1).

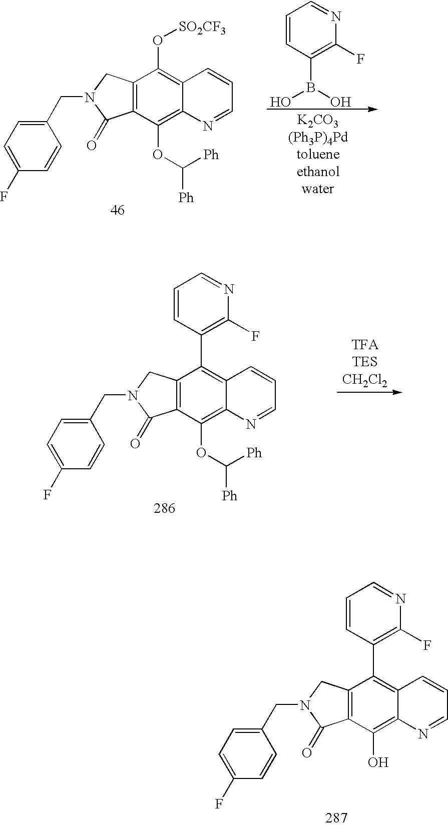

### Example 286

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (40 mg, 0.064 mmol) dissolved in toluene (3 mL)/ethanol (0.6 mL)/water (0.4 mL) was added K₂CO₃ (29 mg, 0.16 mmol), 2-fluoropyridine-3-boronic acid (18 mg, 0.128 mmol) and tetrakis-(triphenylphosphine)-palladium(0) (15 mg, 0.01 mmol). The reaction mixture in the flask was flashed with argon three times. It was then heated to 120° C. under argon 3 hours. The reaction was monitored by TLC (EtOAc/hexane 3/7) (R_{f}46=0.6, R_{f}286=0.1) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by flash chromatography on silica gel with EtOAc/Hexane (1/1) to afford pure 9-benzhydryloxy-7-(4-fluoro-benzyl)-5-(2-fluoro-pyridin-3-yl)-6,7-dihydro-pyrrolo[3,4-g]quinolin-8-one (15), 10.6 mg, 29%.

### Example 287

To a solution of 286 (10.6 mg) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was purified by HPLC to afford 7-(4-fluoro-benzyl)-5-(2-fluoro-pyridin-3-yl)-9-hydroxy-6,7-dihydro-pyrrolo[3,4-g]quinolin-8-one 287, TFA salt, (3.2 mg) as a yellow solid: ¹H NMR (CDCl₃) δ 9.0 (d, 1H), 8.4 (d, 1H), 7.9 (d, 1H), 7.8 (dd, 1H), 7.5 (m, 1H), 7.4 (m, 1H), 7.3 (m, 2H), 7.0 (t, 2H), 4.7 (dd, 2H), 4.2 (dd, 2H); MS: 404 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18(2), 150 mm×21.1 mm.

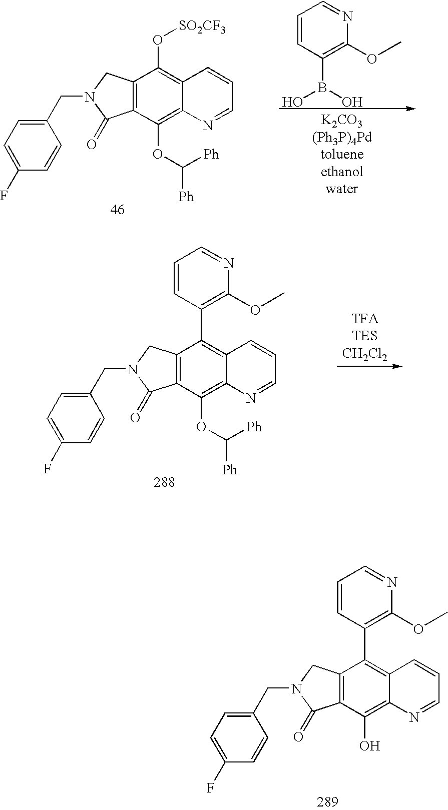

### Example 288

To a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (40 mg, 0.064 mmol) dissolved in toluene (3 mL)/ethanol (0.6 mL)/water (0.4 mL) was added K₂CO₃ (29 mg, 0.16 mmol), 2-methoxypyridine-3-boronic acid (20 mg, 0.128 mmol) and tetrakis-(triphenylphosphine)-palladium(0) (15 mg, 0.01 mmol). The reaction mixture in the flask was flashed with argon three times. It was then heated to 120° C. under argon 3 hours. The reaction was monitored by TLC (EtOAc/hexane 3/7) (R_{f}46=0.6, R_{f}288=0.1) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by flash chromatography on silica gel with EtOAc/Hexane (1/1) to afford pure 9-benzhydryloxy-7-(4-fluoro-benzyl)-5-(2-methoxy-pyridin-3-yl)-6,7-dihydro-pyrrolo[3,4-g]quinolin-8-one (17), 18.0 mg, 48%.

Alternatively, according to a modified Suzuki coupling method of C. H. Chen; Tetrahedron Letter; EN; 44; 5747-5750; 2003, to a solution of trifluoro-methanesulfonic acid 9-benzhydryloxy-7-(4-fluoro-benzyl)-8-oxo-7,8-dihydro-6H-pyrrolo[3,4-g]quinolin-5-yl ester 46 (266.2 mg, 0.428 mmol) dissolved in toluene (5 mL) was added Na₂CO₃ (2M in water, 500 μl), 2-methoxypyridine-3-boronic acid (164 mg, 1.07 mmol) and tetrakis-(triphenylphosphine)-palladium(0) (100 mg, 0.086 mmol). The reaction mixture in the flask was flashed with argon three times. It was then heated to 120° C. under argon 4 hours. The reaction was monitored by TLC (EtOAc/hexane 3/7) (R_{f}1=0.6, R_{f}17=0.1) and LC/MS. After cooling to room temperature, the mixture was diluted with EtOAc (20 mL) and washed with 1N HCl, saturated NaHCO₃ and brine. The organic phase was dried (MgSO₄), filtered and concentrated in vacuo. The residue was purified by flash chromatography on silica gel with EtOAc/Hexane (1/1) to afford pure 9-benzhydryloxy-7-(4-fluoro-benzyl)-5-(2-methoxy-pyridin-3-yl)-6,7-dihydro-pyrrolo[3,4-g]quinolin-8-one 288, 125 mg, 50%. ¹H NMR (CDCl₃) δ 9.0 (dd, 1H), 8.3 (dd, 1H), 8.2 (s, 1H), 7.8 (dd, 4H), 7.7 (dd, 1H), 7.4 (dd, 1H), 7.3-7.1 (m, 8H), 7.0 (m, 2H+1H), 4.7 (dd, 2H), 4.1 (dd, 2H), 3.8 (s, 1H); MS: 582 (M+1).

### Example 289

To a solution of 288 (18 mg) dissolved in dichloromethane (1 mL) was added trifluoroacetic acid (100 μl) and triethylsilane (200 μl). The reaction mixture was stirred at room temperature for ½ hours under an inert atmosphere then concentrated in vacuo. The residue was purified by HPLC to afford 7-(4-fluoro-benzyl)-5-(2-methoxy-pyridin-3-yl)-9-hydroxy-6,7-dihydro-pyrrolo[3,4-g]quinolin-8-one 289, TFA salt, (11.6 mg, 68%) as a yellow solid: ¹H NMR (CDCl₃) δ 9.0 (d, 1H), 8.3 (d, 1H), 7.9 (d, 1H), 7.5 (m, 2H), 7.2 (m, 1H+1H), 7.0 (m, 2H+1H), 4.7 (dd, 2H), 4.1 (dd, 2H), 3.8 (s, 1H); MS: 416 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18(2), 150 mm×21.1 mm.

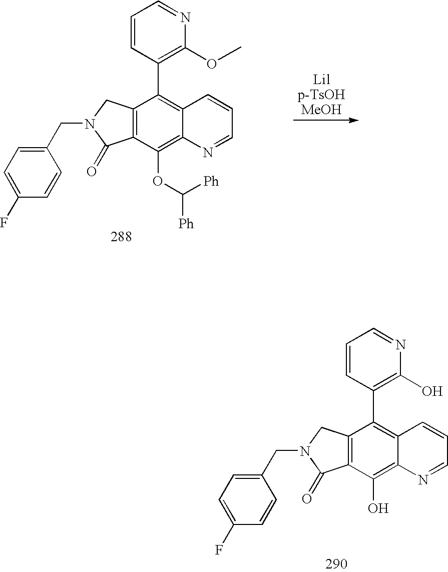

### Example 290

To a solution of 9-benzhydryloxy-7-(4-fluoro-benzyl)-5-(2-methoxy-pyridin-3-yl)-6,7-dihydro-pyrrolo[3,4-g]quinolin-8-one 288 (99 mg, 0.17 mmol) dissolved in methanol (20 mL) was added p-toluenesulfonic acid monohydrate (390 mg, 2.05 mmol) and lithium iodide (1.37 g, 10.26 mmol). The reaction mixture was heated to 120° C. under nitrogen for 10 hours. The reaction was monitored by LC/MS. After cooling to room temperature, the solvent was removed under reduced pressure. The residue was dissolved in 2 mL DMSO and 100 μl of TFA. It was purified by HPLC to afford 7-(4-fluoro-benzyl)-9-hydroxy-5-(2-hydroxy-pyridin-3-yl)-6,7-dihydro-pyrrolo[3,4-g]quinolin-8-one 290, TFA salt, (44.4 mg, 51%) as a yellow solid: ¹H NMR (CD₃OD) δ 8.9 (dd, 1H), 8.2 (dd, 1H), 7.7 (m, 1H+1H), 7.6 (d, 2H), 7.4 (m, 2H), 7.1 (m, 2H), 6.6 (t, 1H), 4.8 (dd, 2H), 4.3 (d, 2H); MS: 402 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18(2), 150 mm×21.1 mm.

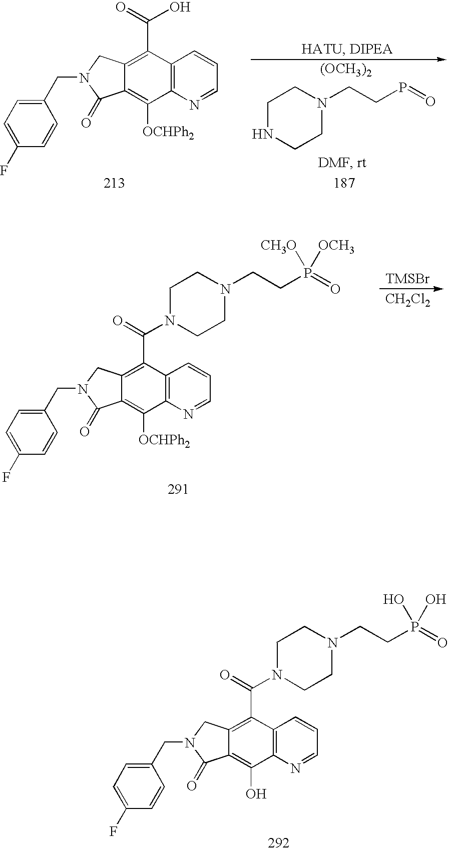

### Example 291

To a solution of the trifluoroacetate salt of (2-piperazin-1-yl-ethyl)-phosphonic acid dimethyl ester 187 (0.023 g, 0.077 mmol) in 1 ml DMF was added diisopropylethylamine (33 μL, 0.192 mmol). This mixture was added to a solution of 9-benzhydryloxy-7-(4-fluoro-benzyl)-8-oxo-7,8-dihydro-6H-pyrrolo[3,4-g]quinoline-5-carboxylic acid 213 (0.020 g, 0.038 mmol) that had been mixed with HATU (0.0293 g, 0.077 mmol) in 1 ml of DMF. The reaction was stirred at rt under inert atmosphere for 3 h, at which time TLC in 100% EtOAc showed complete consumption of starting material. The reaction mixture was introduced directly onto silica gel (99/1 EtOH/Et₃N) to give 20 mg of (2-{4-[9-benzhydryloxy-7-(4-fluoro-benzyl)-8-oxo-7,8-dihydro-6H-pyrrolo[3,4-g]quinoline-5-carbonyl]-piperazin-1-yl}-ethyl)-phosphonic acid dimethyl ester 291 after flash chromatography.

### Example 292

An excess of trimethylsilyl bromide (TMSBr, 0.015 g, 0.1 mmol) was added to (2-{4-[9-benzhydryloxy-7-(4-fluoro-benzyl)-8-oxo-7,8-dihydro-6H-pyrrolo[3,4-g]quinoline-5-carbonyl]-piperazin-1-yl}-ethyl)-phosphonic acid dimethyl ester 291 in 1 mL of CH₂Cl₂. After stirring at room temperature (rt) for 16 h, volatiles were removed under vacuum and the residue was triturated with Et₂O to provide pure the HBr salt of (2-{4-[7-(4-Fluoro-benzyl)-9-hydroxy-8-oxo-7,8-dihydro-6H-pyrrolo[3,4-g]quinoline-5-carbonyl]-piperazin-1-yl}-ethyl)-phosphonic acid 292 (12 mg, 95%) as a yellow solid. ¹H NMR (DMSO) δ: 8.95 (d, 1H), 8.75 (d, 1H), 8.54 (1H, d), 8.35 (bm, 1H), 7.78 (m, 2H), 7.52 (m, 2H), 7.4-7.32 (bm, 2H), 7.15 (t, 2H), 4.85 (bm, 1H) 4.45 (bm, 2H) 2.04 (bm, 2H); ³¹P NMR (DMSO) δ 19.9; MS: 529 (M+H).

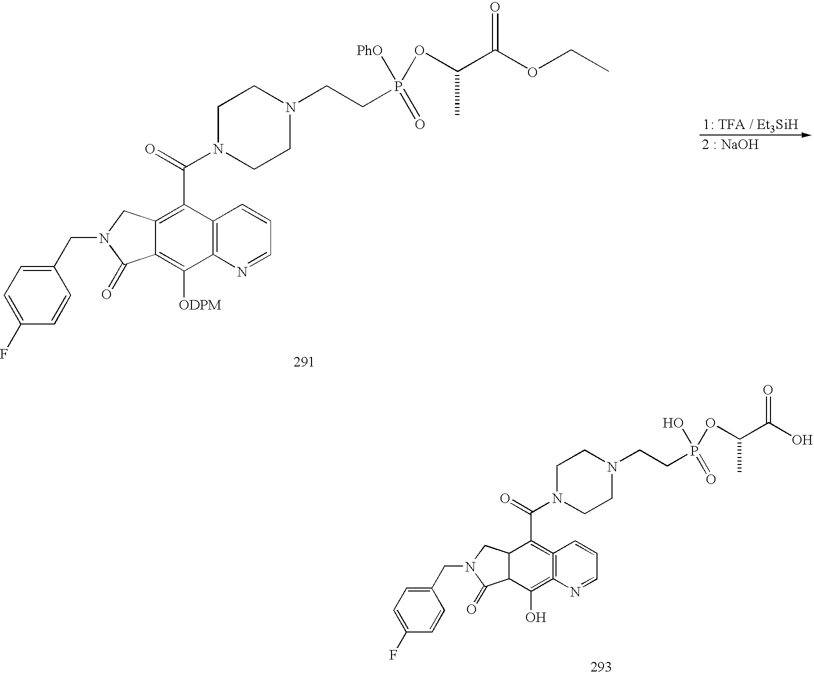

### Example 293

To a solution of 2-[(2-{4-[9-benzhydryloxy-7-(4-fluoro-benzyl)-8-oxo-7,8-dihydro-6H-pyrrolo[3,4-g]quinoline-5-carbonyl]-piperazin-1-yl}-ethyl)-phenoxy-phosphinoyloxy]-propionic acid ethyl ester 223 (15 mg, 0.017 mmol) in 1 ml CH₂Cl₂ at rt was added an excess of TFA (10 μL, 0.085 mmol) and triethylsilane (30 μL, 0.17 mmol). The reaction was stirred under N₂ with monitoring via LC/MS. After 8 h, the volatiles were removed by vacuum and the residue dissolved in 1 mL of a 1/1 mixture of acetonitrile/water. 50 μL of 1M NaOH was added and the reaction was stirred at rt overnight. At this time, the product was introduced directly onto reverse phase HPLC to afford, after lyophilization, 2-[(2-{4-[7-(4-fluoro-benzyl)-9-hydroxy-8-oxo-7,8-dihydro-6H-pyrrolo[3,4-g]quinoline-5-carbonyl]-piperazin-1-yl}-ethyl)-hydroxy-phosphinoyloxy]-propionic acid as the trifluoroacetate salt, 293 (5 mg, 39%). ¹H NMR (D₂O) δ: 9.10 (d, 1H), 8.95-8.72 (bm, 1H), 8.14 (bs, 1H), 7.20-7.3 (bm, 2H), 6.92-7.08 (bs, 2H), 4.65-4.25 (m, 4H), 3.78-3.65 (bs, 1H), 3.62-3.10 (bm, 9H), 2.75 (d, 2H), 1.95 (m, 2H), 1.35 (d, 3H); ³¹P NMR (D₂O) δ 19.5; MS: 629 (M+H).

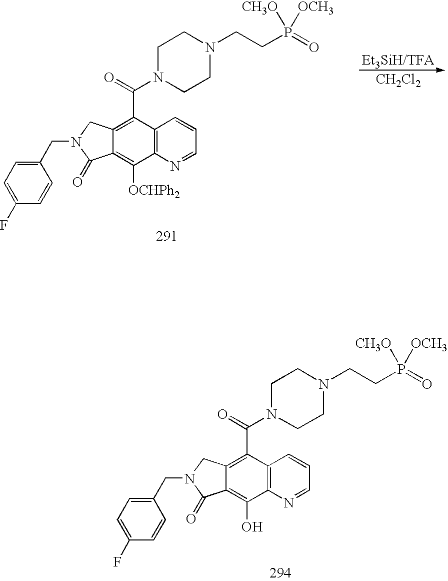

### Example 294

To (2-{4-[9-benzhydryloxy-7-(4-fluoro-benzyl)-8-oxo-7,8-dihydro-6H-pyrrolo[3,4-g]quinoline-5-carbonyl]-piperazin-1-yl}-ethyl)-phosphonic acid dimethyl ester 291 (5 mg, 0.0069 mmol) in 1 mL CH₂Cl₂ is added CF₃CO₂H (6 μL, 0.035 mmol) and triethylsilane (12 μL, 0.07 mmol). After 2 h, the volatile reaction components were removed by vacuum and the residue was washed with diethyl ether to give (2-{4-[7-(4-fluoro-benzyl)-9-hydroxy-8-oxo-7,8-dihydro-6H-pyrrolo[3,4-g]quinoline-5-carbonyl]-piperazin-1-yl}-ethyl)-phosphonic acid 294 as the trifluoroacetate salt (4.5 mg, 97%): ¹H NMR (CD₃OD) δ: 8.90 (d, 0.7H) 8.74 (d, 0.3H), 8.45 (d, 0.3H), 8.31 (d, 0.7H), 7.75 (dd, 0.7H), 7.55 (dd, 0.3H), 7.40 (m, 2H), 7.12 (m, 2H), 4.54 (s, 2H), 4.15 (bs, 1H), 3.85 (s, 3H), 3.75 (s, 3H), 3.62-3.40 (bs, 2H), 3.12 (bs, 2H), 2.45-2.30 (m, 2H); ¹⁹F NMR (CD₃OD) δ ⁻78, ⁻127; ³¹P NMR (CD₃OD) δ 29; MS: 556 (M+H).

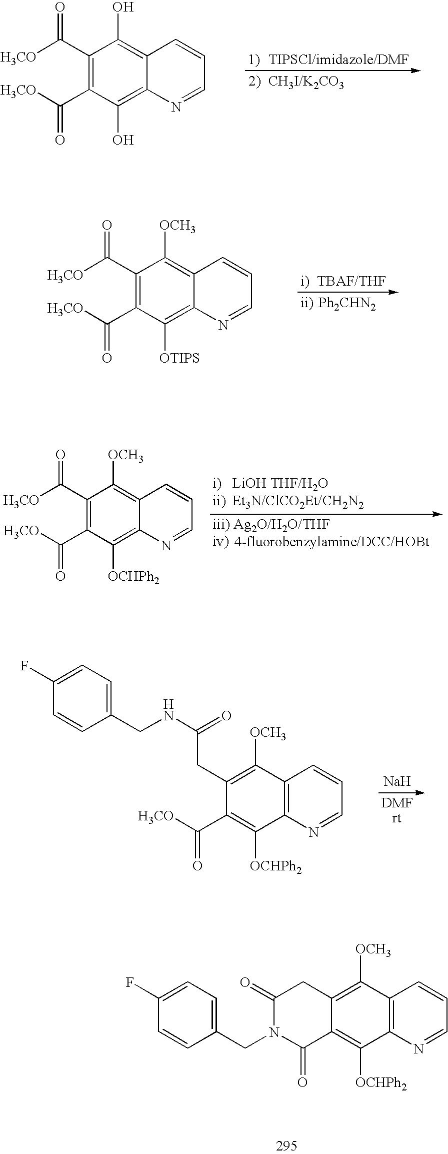

### Example 295

Imidazole (0.74 g, 10.8 mmol) and chlorotriisopropylsilane (TIPSCl, 1.15 g, 6.0 mmol) were added to 5,8-dihydroxy-quinoline-6,7-dicarboxylic acid dimethyl ester (prepared by the method in Oguchi, S. Bulletin of the Chemical Society of Japan 1974, 47, 1291, 1.5 g, 5.4 mmol) in 20 mL DMF. The reaction was stirred for 48 h at rt and then the reaction was partitioned between 0.5 L methyl t-butyl ether and 150 mL saturated aq. LiCl. The organic layer was dried over Na₂SO₄ and the solvent removed by rotary evaporation. The residue (1.4 g, 3.2 mmol) was redissolved in 25 mL DMF and treated with K₂CO₃ (0.66 g, 4.8 mmol) followed by methyl iodide (MeI, 0.6 g, 4.8 mL) at rt. After 2 h, the reaction mixture was concentrated and purified by introduction of the reaction mixture onto silica gel for flash chromatography (4/1 hexanes/ethyl acetate) to give 5-methoxy-8-triisopropylsilanyloxy-quinoline-6,7-dicarboxylic acid dimethyl ester (1.4 g, 59% overall yield): ¹H NMR (CDCl₃) δ 8.85 (d, 1H), 8.45 (d, 1H), 7.50 (dd, 1H), 4.05 (s, 3H), 3.95 (s, 3H), 3.90 (s, 3H), 1.45 (septet, 3H), 1.05 (d, 9H); MS: 448 (M+H).

A 1M solution of TBAF in THF (4 ml) was added to 5-methoxy-8-triisopropylsilanyloxy-quinoline-6,7-dicarboxylic acid dimethyl ester (0.85 g, 1.9 mmol) in 20 ml dry THF. The reaction was stirred at rt for 1 h, at which time the reaction mixture was concentrated and the residue dissolved in 100 mL diethyl ether and washed with 25 mL 1N HCl, followed by 25 mL of saturated aq. NaCl. The organic layer was concentrated and the residue was dissolved in 40 mL dichloroethane. Diphenyldiazomethane (0.7 g, 3.8 mmol) was added and the reaction temperature was raised to 70° C. for 24 h. The reaction mixture was concentrated and the residue chromatographed on silica gel (1/1 hexanes/EtOAc) to give 8-benzhydryloxy-5-methoxy-quinoline-6,7-dicarboxylic acid dimethyl ester (0.8 g, 61% yield overall). ¹H NMR (CDCl₃) δ 8.85 (d, 1H), 8.45 (d, 1H), 7.45 (dd, 1H), 3.98 (s, 3H), 3.85 (s, 3H), 3.74 (s, 3H); MS: 480 (M+Na).

Lithium hydroxide (LiOH, 0.07 g, 2.95 mmol) was added to 8-benzhydryloxy-5-methoxy-quinoline-6,7-dicarboxylic acid dimethyl ester (0.27 g, 0.59 mmol) in 1 mL 3/1 THF/H₂O. The reaction was heated at 45° C. and after 24 h, the reaction was diluted with 50 mL dichloromethane and acidified with 1 mL 0.1 M HCl. The organic layer was dried over Na₂SO₄ and concentrated to give 180 mg of an oil which was dissolved in 5 mL THF, triethylamine (0.168 g, 1.2 mmol) and ethyl chloroformate (0.064 g, 0.6 mmol). After 2 h, the reaction was diluted with diethyl ether and washed with brine. The organic layer was dried over Na₂SO₄ and the organic layer decanted from drying agent. The ether layer was cooled to 0° C. and a solution of ca. 0.3 M diazomethane in diethyl ether (4 mL, ca. 1.2 mmol) was added dropwise. After stirring for 24 h to effect diazotization, the ether layer was removed along with excess diazomethane via rotary evaporation. The resulting residue was dissolved in 4 mL of 1/1 THF/water, and silver(I) oxide (0.035 g, 0.15 mmol) was added. The reaction was heated to 60° C. for a period of 4 h, then the reaction mixture was diluted with 50 mL EtOAc and acidified with 10 ml 1N HCl. The organic layer was dried over Na₂SO₄ and concentrated. The resulting residue was then taken up in 2 mL THF, and treated with hydroxybenzotriazole (HOBt, 0.08 g, 0.6 mmol), dicyclohexylcarbodiimide (DCC, 0.12 g, 0.6 mmol) and 4-fluorobenzylamine (0.07 g, 0.6 mmol). After a period of 16 h, the reaction was introduced directly to chromatography on silica gel (100% diethyl ether) to give 8-benzhydryloxy-6-[(4-fluoro-benzylcarbamoyl)-methyl]-5-methoxy-quinoline-7-carboxylic acid methyl ester (0.12 g, 38% overall yield): ¹H NMR (CDCl₃) δ 8.85 (d, 1H), 8.35 (d, 1H), 7.60-6.8 (cm, 12H), 6.15 (s, 1H), 4.30 (m, 2H), 3.95 (s, 3H), 3.75 (s, 3H), 3.65 (s, 2H), 3.54 (t, 1H); MS: 587 (M+Na).

A 60% sodium hydride (NaH) mineral oil dispersion (0.002 g, 0.06 mmol was added to a solution of 8-benzhydryloxy-6-[(4-fluoro-benzylcarbamoyl)-methyl]-5-methoxy-quinoline-7-carboxylic acid methyl ester (0.020 g, 0.04 mmol) in 1 mL of anhydrous DMF. The resulting indigo-tinted solution was stirred at rt for a period of 30 min, and then diluted with diethyl ether (50 ml) and washed with sat. aq. NH₄Cl (25 mL). The organic layer was dried over Na₂SO₄ and solvent was removed by rotary evaporation. The residue was purified by silica gel chromatography (1/1 hexanes/diethyl ether and then 100% MeOH to elute product fractions) to give 9-benzhydryloxy-7-(4-fluoro-benzyl)-10-methoxy-5H-1,7-diaza-anthracene-6,8-dione 295 (9 mg, 48%).

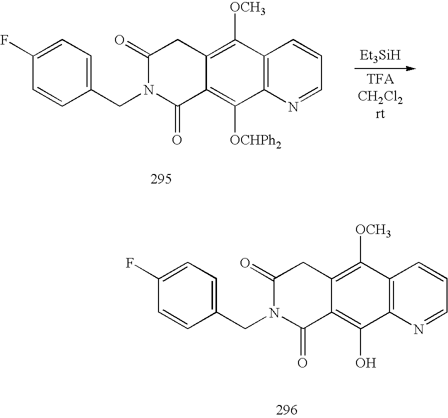

### Example 296

9-Benzhydryloxy-7-(4-fluoro-benzyl)-10-methoxy-5H-1,7-diaza-anthracene-6,8-dione 295 (6 mg, 0.01 mmol) in 1 mL CH₂Cl₂ was treated with 0.1 mL trifluoroacetic acid and 0.05 mL triethylsilane. After 1 h, volatiles were removed and the product was purified via trituration with diethyl ether to give the trifluoroacetate salt of 7-(4-Fluoro-benzyl)-9-hydroxy-10-methoxy-5H-1,7-diaza-anthracene-6,8-dione 296 (5 mg, 62%): ¹H NMR (CDCl₃) δ 12.98 (s, 1H), 9.10 (d, 1H), 8.35 (d, 1H), 7.65 (m, 1H), 7.55 (m, 2H), 7.04 (t, 2H), 5.2 (s, 2H), 4.75 (s, 1H), 4.20 (s, 1H), 3.95 (s, 3H); MS: 367 (M+Na).

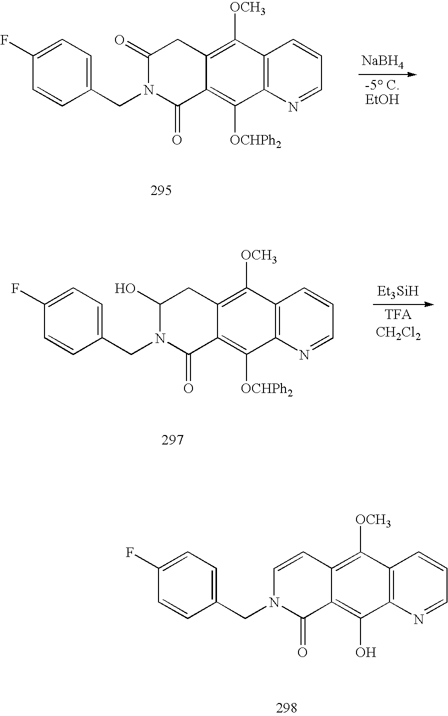

### Example 297

Sodium borohydride (NaBH₄, 0.021 g, 0.56 mmol) was added to 9-benzhydryloxy-7-(4-fluoro-benzyl)-10-methoxy-5H-1,7-diaza-anthracene-6,8-dione 295 (30 mg, 0.056 mmol) in 1 mL EtOH at −5° C. The reaction was stirred at low temperature for a period of 2 h, then the reaction was diluted with CH₂Cl₂ (25 mL) and washed with 10 mL sat. aq. sodium bicarbonate solution. The aqueous layer was then washed twice with 25 ml portions of CH₂Cl₂ and the combined organic layers washed with brine and dried over Na₂SO₄. The reduction product was purified on silica gel (100% Et₂O) to give 6 mg of 9-benzhydryloxy-7-(4-fluoro-benzyl)-10-methoxy-5H-1,7-diaza-anthracene-6-hydroxy, 8-one 297.

### Example 298

9-Benzhydryloxy-7-(4-fluoro-benzyl)-10-methoxy-5H-1,7-diaza-anthracene-6-hydroxy, 8-one 297 (6 mg, 0.01 mmol) was dissolved in 1 mL CH₂Cl₂ and treated with 0.1 mL trifluoroacetic acid and 0.1 mL triethylsilane. After 1 hr, volatiles were removed and the product was purified via trituration with diethyl ether to give the trifluoroacetate salt of 7-(4-Fluoro-benzyl)-9-hydroxy-10-methoxy-7H-1,7-diaza-anthracen-8-one 298 (2 mg, 38%). ¹H NMR (CD₃OD) δ 9.35 (d, 1H), 8.75 (d, 1H), 7.80 (dd, 1H), 7.33 (m, 2H), 7.08 (m, 3H), 6.85 (d, 1H), 5.15 (s, 2H), 3.95 (s, 3H). MS: 351 (M+H).

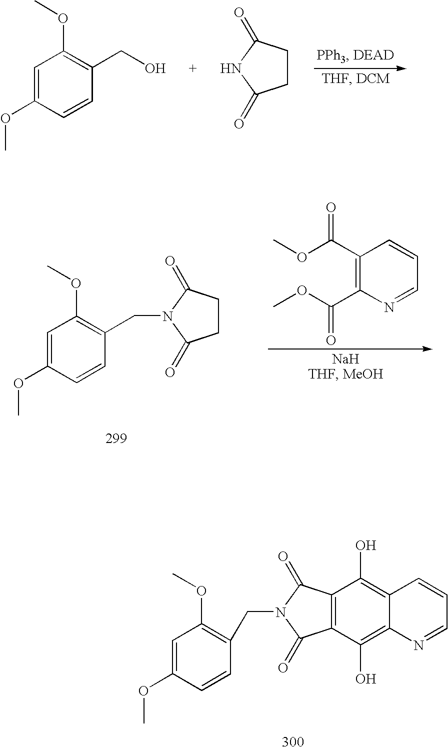

### Example 299

To 2,4-dimethoxybenzyl-alcohol (4.3 g, 25.6 mmol) and pyrrolidine-2,5-dione (succinimide, 1.2 g, 12.2 mmol) dissolved in tetrahydrofuran (25 ml) and dichloromethane (25 ml) was added triphenyphosphine (6.4 g, 24.4 mmol). After cooling to 0° C., diethylazidodicarboxylate (DEAD, 4.25 g, 24.4 mmol) was added dropwise to the reaction mixture. The reaction mixture was then allowed to warm to room temperature and kept at room temperature with stirring overnight. Following concentration in vacuo, 100 ml of a (1:1)hexane/ether solution was added and this mixture was stored at 0° C. overnight. The resulting solid precipitate was filtered off and the filtrate was concentrated in vacuo. The resulting residue was purified by silica gel chromatography (3/1—ethyl acetate/hexane) to afford 1-(2,4-dimethoxy-benzyl)-pyrrolidine-2,5-dione 299 (1.4 g, 5.6 mmol, 46%). ¹H NMR (CDCl₃) δ 7.07 (d, 1H), 6.38 (m, 2H), 4.60 (s, 2H), 3.76 (s, 3H), 2.62 (s, 4H).

### Example 300

To 1-(2,4-dimethoxy-benzyl)-pyrrolidine-2,5-dione 299 (1.4 g, 5.6 mmol) and pyridine-2,3-dicarboxylic acid dimethyl ester (1.13 g, 5.8 mmol) dissolved in tetrahydrofuran (60 ml) and methanol (7.0 ml) was added a 60% dispersion of sodium hydride in mineral oil (NaH, 492 mg, 12.3 mmol). The reaction mixture was warmed to 80° C. and kept at 80° C. with stirring overnight. The reaction mixture was placed in an ice bath and titrated to a pH of 4 with 1 M HCl. 200 ml of ether was added and the resulting yellow solid was collected by filtration. The solid was washed twice with ether, twice with water, and dried under high vacuum with heating to provide 7-(2,4-dimethoxy-benzyl)-5,9-dihydroxy-pyrrolo[3,4-g]quinoline-6,8-dione 300 (1.1 g, 52%). ¹H NMR (d-DMSO) δ 10.8 (broad, 2H), 9.0 (d, 1H), 8.67 (d, 1H), 7.72 (m, 1H), 6.90 (d, 1H), 6.5 (d, 1H), 6.38 (dd, 1H), 4.58 (s, 2H), 3.76 (s, 3H), 3.66 (s, 3H). MS: 382.1 (M+1)

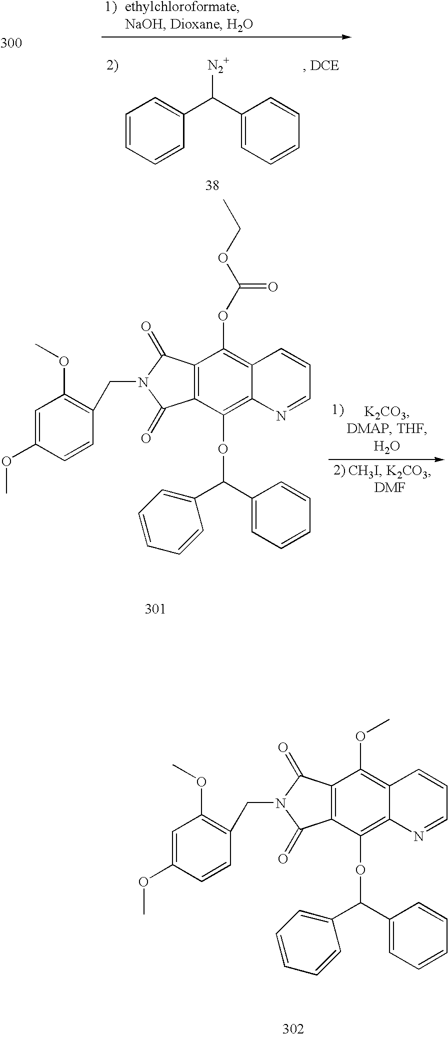

### Example 301

7-(2,4-Dimethoxy-benzyl)-5,9-dihydroxy-pyrrolo[3,4-g]quinoline-6,8-dione 300 (1.1 g, 2.9 mmol) was dissolved in dioxane (14.5 ml) and H₂O (9.7 ml) and cooled to 0° C. To this reaction mixture was added 1.0 M NaOH (5.8 ml, 5.8 mmol), followed by ethylchloroformate (347.3 mg, 3.2 mmol). After stirring at 0° C. for 30 minutes, dioxane (10 ml) and ethylchloroformate (51 mg, 0.5 mmol) were added and the reaction stirred for another 30 minutes at 0° C. The reaction mixture was quenched with the addition of acetic acid (0.6 ml) and concentrated in vacuo. The crude mixture was diluted with ethyl acetate and washed once with 5% citric Acid (aqueous), twice with water, once with brine, and dried over magnesium sulfate. The resulting residue was dissolved in 1,2-dichloroethane (30 ml) and diphenyl-methanediazonium 38 (diphenyldiazomethane, 1.1 g, 5.6 mmol) was added. The reaction mixture was then stirred overnight at room temperature. Following dilution with dichloromethane, the reaction mixture was washed with once with water, once with brine, dried over magnesium sulfate, and concentrated in vacuo. The residue was purified by silica gel chromatography (1/1—Hexanes/Ethyl Acetate) to afford carbonic acid 9-benzhydryloxy-7-(2,4-dimethoxy-benzyl)-6,8-dioxo-7,8-dihydro-6H-pyrrolo[3,4-g]quinolin-5-yl ester ethyl ester 301 (1.2 g, 1.9 mmol, 66%). ¹H NMR (CDCl₃) δ 9.10 (dd, 1H), 8.40 (dd, 1H), 7.95 (s, 1H), 7.68 (m, 1H), 7.60 (d, 4H), 7.15 (m, 6H), 7.0 (d, 1H), 6.40 (d, 1H), 6.36 (d, 1H), 4.80 (s, 2H), 4.35 (q, 2H), 3.75 (s, 3H), 3.73 (s, 3H), 1.31 (t, 3H). MS: 641.2 (M+23).

### Example 302

Potassium carbonate (2.6 g, 19.0 mmol) and N,N-dimethyl-aminopyridine (DMAP, 0.464 g, 3.8 mmol) were added to carbonic acid 9-benzhydryloxy-7-(2,4-dimethoxy-benzyl)-6,8-dioxo-7,8-dihydro-6H-pyrrolo[3,4-g]quinolin-5-yl ester ethyl ester 301 (1.2 g, 1.9 mmol) dissolved in tetrahydrofuran (40 ml) and water (20 ml) was added. After stirring overnight at room temperature, the reaction mixture was concentrated in vacuo and diluted with ethyl acetate. It was washed twice with 5% citric Acid (aqueous), twice with water, once with brine, dried over magnesium sulfate, and concentrated in vacuo. The resulting residue was dissolved in dimethylformamide (10 ml). To this reaction mixture was added potassium carbonate (1.24 g, 9.0 mmol) and iodomethane (methyl iodide, MeI, 2.55 g, 18.0 mmol). After stirring overnight at room temperature, the reaction mixture was diluted with ethyl acetate, washed twice with 5% citric acid, twice with water, once with brine, and concentrated in vacuo to afford 9-benzhydryloxy-7-(2,4-dimethoxy-benzyl)-5-methoxy-pyrrolo[3,4-g]quinoline-6,8-dione 302 (1.1 g, 1.9 mmol, 100%). ¹H NMR (d-DMSO) δ 9.16 (dd, 1H), 8.60 (dd, 1H), 7.82 (s, 1H), 7.75 (m, 1H), 7.54 (d, 4H), 7.16 (m, 6H), 6.82 (d, 1H), 6.56 (d, 1H), 6.44 (dd, 1H), 4.66 (s, 2H), 4.10 (s, 3H), 3.76 (s, 3H), 3.70 (s, 3H). MS: 583.2 (M+23).

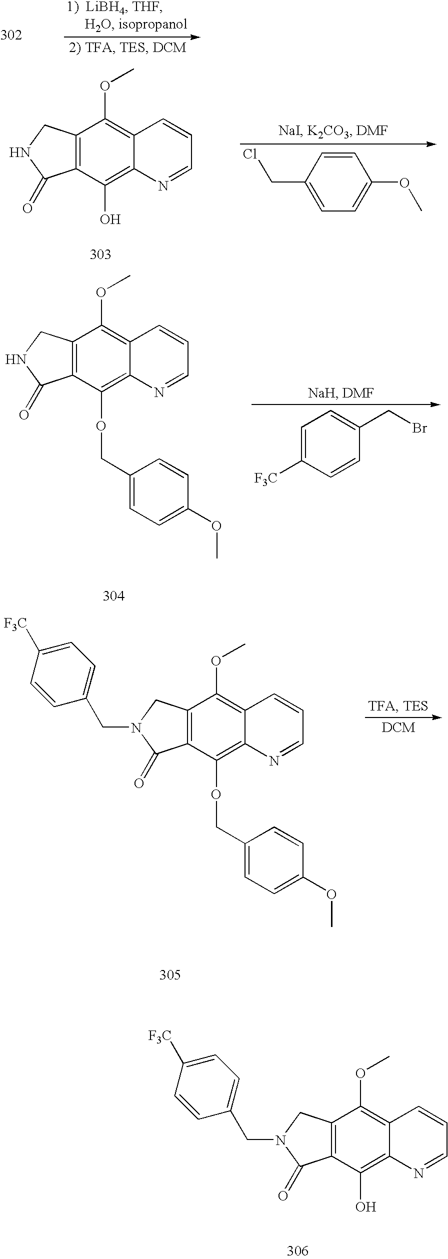

### Example 303

9-Benzhydryloxy-7-(2,4-dimethoxy-benzyl)-5-methoxy-pyrrolo[3,4-g]quinoline-6,8-dione 302 (500 mg, 0.89 mmol) was dissolved in tetrahydrofuran (6.0 ml), water (1.2 ml), and isopropanol (2.4 ml) and cooled to 0° C. Lithium borohydride (LiBH₄, 96.9 mg, 4.45 mmol) was then added and the reaction mixture was removed from the ice bath and stirred at room temperature for 2 hours. After quenching with acetic acid (0.5 ml), the reaction mixture was diluted with ethyl acetate, washed with twice with water, once with brine, and concentrated in vacuo. The resulting residue was dissolved in dichloromethane (9.2 ml) and triethylsilane (1.8 ml), and cooled to 0° C. After adding trifluoroacetic acid (3.6 ml), the reaction mixture was warmed to room temperature and stirred at room temperature for 1 hour. The mixture was concentrated in vacuo and the resulting residue was redissolved in trifluoroacetic acid (10 ml) and triethylsilane (2 ml). It was then warmed to 75° C. and stirred at 75° C. for 2 hours. The reaction mixture was concentrated in vacuo and azeotroped three times with a (1:1) toluene/tetrahydrofuran solution. The resulting residue was triturated three times with a (3:1) hexane/ether mixture. The remaining solid in the filter funnel and reaction flask was dissolved in methanol, combined, and concentrated in vacuo to afford 9-hydroxy-5-methoxy-6,7-dihydro-pyrrolo[3,4-g]quinolin-8-one 303 (240 mg, 113%). ¹H NMR (d-DMSO) δ 8.84 (dd, 1H), 8.58 (broad, 1H), 8.50 (dd, 1H), 7.60 (m, 1H), 4.60 (s, 2H), 3.94 (s, 3H). MS: 231.1 (M+1).

### Example 304

Potassium carbonate (60.1 mg, 0.435 mmol), 4-methoxybenzylchloride (41 mg, 0.26 mmol), and sodium iodide (6.3 mg, 0.043 mmol) were added to 9-hydroxy-5-methoxy-6,7-dihydro-pyrrolo[3,4-g]quinolin-8-one 303 (20 mg, 0.087 mmol) dissolved in dimethylformamide 8 (0.4 ml). The reaction mixture was warmed to 60° C. and stirred at 60° C. for one hour. After cooling the reaction mixture to 0° C., acetic acid (0.06 ml) was added and the mixture was concentrated in vacuo. The residue was diluted with ethyl acetate and washed once with 5% Citric Acid, twice with water, once with brine, and concentrated in vacuo. The residue was purified by silica gel chromatography (9/1—dichloromethane/methanol) to afford 5-methoxy-9-(4-methoxy-benzyloxy)-6,7-dihydro-pyrrolo[3,4-g]quinolin-8-one 304 (16 mg, 0.046 mmol, 53%). ¹H NMR (CDCl₃) δ 9.0 (dd, 1H), 8.64 (dd, 1H), 7.51 (d, 2H), 7.46 (m, 1H), 6.80 (d, 2H), 6.50 (broad, 1H), 5.60 (s, 2H), 3.98 (s, 3H), 3.72 (s, 3H). MS: 351.1 (M+1).

### Example 305

5-Methoxy-9-(4-methoxy-benzyloxy)-6,7-dihydro-pyrrolo[3,4-g]quinolin-8-one 304 (25.4 mg, 0.073 mmol,) was dissolved in dimethylformamide (0.4 ml) and cooled to 0° C. Sodium hydride (3.6 mg, 0.095 mmol) was added, followed by stirring at 0° C. for 5 minutes. 4-trifluoromethyl-benzylbromide (21.0 mg, 0.088 mmol) was added and the reaction mixture was allowed to warm to room temperature and kept at room temperature with stirring for 5 minutes. It was cooled to 0° C., quenched with acetic acid (0.030 ml), and concentrated in vacuo. The mixture was diluted with ethyl acetate, washed twice with water. once with brine, dried over magnesium sulfate, and concentrate in vacuo. The residue was purified by silica gel chromatography (99/1—ethyl acetate/acetic acid) to afford 5-Methoxy-9-(4-methoxy-benzyloxy)-7-(4-trifluoromethyl-benzyl)-6,7-dihydro-pyrrolo[3,4-g]quinolin-8-one 305 (13 mg, 0.026 mmol, 35%). ¹H NMR δ 9.15 (dd, 1H), 8.60 (dd, 1H), 7.60 (m, 4H), 7.40 (d, 2H), 6.80 (d, 2H), 5.85 (s, 2H), 4.80 (s, 2H), 4.42 (s, 2H), 3.98 (s, 3H), 3.86 (s, 3H). MS: 509.2 (M+1).

### Example 306

To 5-methoxy-9-(4-methoxy-benzyloxy)-7-(4-trifluoromethyl-benzyl)-6,7-dihydro-pyrrolo[3,4-g]quinolin-8-one 305 (13 mg, 0.026 mmol) dissolved in dichloromethane (0.200 ml) was added triethylsilane (TES, 0.05 ml) and trifluoroacetic acid (TFA, 0.100 ml). After stirring at room temperature for 15 minutes, the reaction mixture was concentrated in vacuo and azeotroped three times with a (1:1) tetrahydrofuran to toluene mixture. The resulting residue was then triturated three times with a (3:1) hexane to ether mixture to afford 9-hydroxy-5-methoxy-7-(4-trifluoromethyl-benzyl)-6,7-dihydro-pyrrolo[3,4-g]quinolin-8-one 306 (7 mg, 0.014 mmol, 54%). ¹H NMR (CD₃OD) δ 9.0 (dd, 1H), 8.58 (dd, 1H), 7.60 (d, 2H), 7.40 (d, 2H), 4.80 (s, 2H), 4.50 (s, 2H), 3.95 (s, 3H). ¹⁹F NMR δ −63, −76.2. MS: 389.1 (M+1).

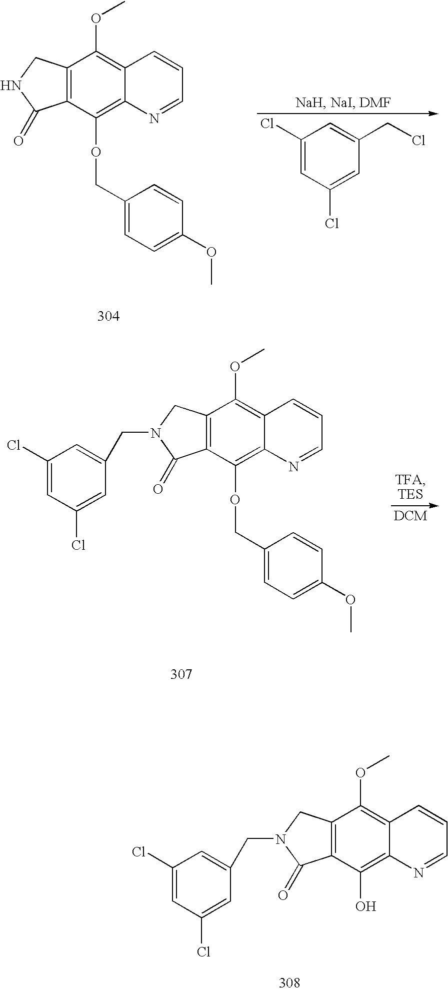

### Example 307

5-Methoxy-9-(4-methoxy-benzyloxy)-6,7-dihydro-pyrrolo[3,4-g]quinolin-8-one 304 (17 mg, 0.049 mmol) was dissolved in dimethylformamide (0.3 ml) and cooled to 0° C. After adding sodium hydride (2.5 mg, 0.064 mmol), the reaction was stirred for 5 minutes at 0° C. 3,5-Dichlorobenzylchloride (11.5 mg, 0.059 mmol) and a catalytic amount of sodium iodide were then added. The reaction mixture was warmed to room temperature and stirred at room temperature for 30 minutes. It was then cooled to 0° C., acidified with acetic acid (0.030 ml), and concentrated in vacuo. The resulting residue was diluted with ethyl acetate, washed twice with water, once with brine, and concentrated in vacuo. The residue was purified by silica gel chromatography (99/1—ethyl acetate/acetic acid) to afford 7-(3,5-dichloro-benzyl)-5-methoxy-9-(4-methoxy-benzyloxy)-6,7-dihydro-pyrrolo[3,4-g]quinolin-8-one 307 (7 mg, 40%). ¹H NMR (CDCl₃) δ 9.0 (dd, 1H), 8.40 (dd, 1H), 7.60 (d, 2H), 7.55 (m, 1H), 7.20 (m, 3H), 6.80 (d, 2H), 5.60 (s, 2H), 4.75 (s, 2H), 4.40 (s, 2H), 3.95 (s, 3H), 3.75 (s, 3H). MS: 509.1 (M+1).

### Example 308

In a manner similar to the protocol described in Example 306, 7-(3,5-dichloro-benzyl)-5-methoxy-9-(4-methoxy-benzyloxy)-6,7-dihydro-pyrrolo[3,4-g]quinolin-8-one 307 (17 mg, 0.049 mmol) was deprotected to provide 7-(3,5-dichloro-benzyl)-9-hydroxy-5-methoxy-6,7-dihydro-pyrrolo[3,4-g]quinolin-8-one 308 (10 mg, 0.020 mmol, 41%). ¹H NMR (CD₃OD) δ 9.0 (dd, 1H), 8.60 (d, 1H), 7.60 (m, 1H), 7.20 (m, 3H) 4.75 (s, 2H), 4.45 (s, 2H), 4.0 (s, 3H). ¹⁹F δ −76. MS: 390.1 (M+1).

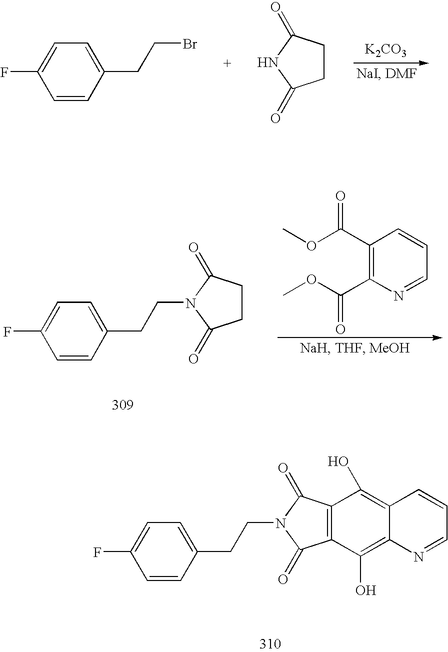

### Example 309

To a solution of 1-(2-bromo-ethyl)-4-fluoro-benzene (587 mg, 3.7 mmol) and pyrrolidine-2,5-dione (succinimide, 733.3 mg, 7.4 mmol) in dimethylformamide (15 ml) was added potassium carbonate (2.0 g, 14.8 mmol) and sodium iodide (277 mg, 1.9 mmol). The reaction mixture was warmed to 60° C. and kept at 60° C. overnight with stirring. The reaction mixture was cooled to room temperature and concentrated in vacuo. The concentrate was diluted with ethyl acetate and washed twice with a saturated sodium bicarbonate aqueous solution, twice with water, once with brine, and concentrated in vacuo. The residue was purified by silica gel chromatography (100% ethylacetate) to afford 1-[2-(4-fluoro-phenyl)-ethyl]-pyrrolidine-2,5-dione 309 (570 mg, 2.6 mmol, 70%) as a solid. ¹H NMR (CDCl₃) δ 7.14 (m, 2H), 6.94 (t, 2H), 3.68 (t, 2H), 2.84 (t, 2H), 2.63 (s, 4H).

### Example 310

To 1-[2-(4-Fluoro-phenyl)-ethyl]-pyrrolidine-2,5-dione 309 (270 mg, 1.22 mmol and pyridine-2,3-dicarboxylic acid dimethyl ester (261.6 mg, 1.34 mmol) dissolved in tetrahydrofuran (12.0 ml) and methanol (1.4 ml) was added a 60% dispersion of sodium hydride in mineral oil (108 mg, 2.7 mmol). The reaction mixture was warmed to 80° C. and kept at 80° C. with stirring overnight. The reaction mixture was then placed in an ice bath and titrated to a pH of 4 with 1 M HCl. Two hundred (200) ml of diethylether was then added and the resulting yellow solid was collected by filtration. The solid was washed twice with ether, twice with water, and dried under high vacuum with heating to provide 7-[2-(4-fluoro-phenyl)-ethyl]-5,9-dihydroxy-pyrrolo[3,4-g]quinoline-6,8-dione 310 (250 mg, 0.71 mmol, 58%). ¹H NMR (d-DMSO) δ 10.7 (broad, 1H), 8.98 (dd, 1H), 8.66 (dd, 1H), 7.73 (m, 1H), 7.18 (m, 2H), 7.04 (t, 2H), 3.72 (t, 2H), 2.86 (t, 2H). MS: 353.1 (M+1).

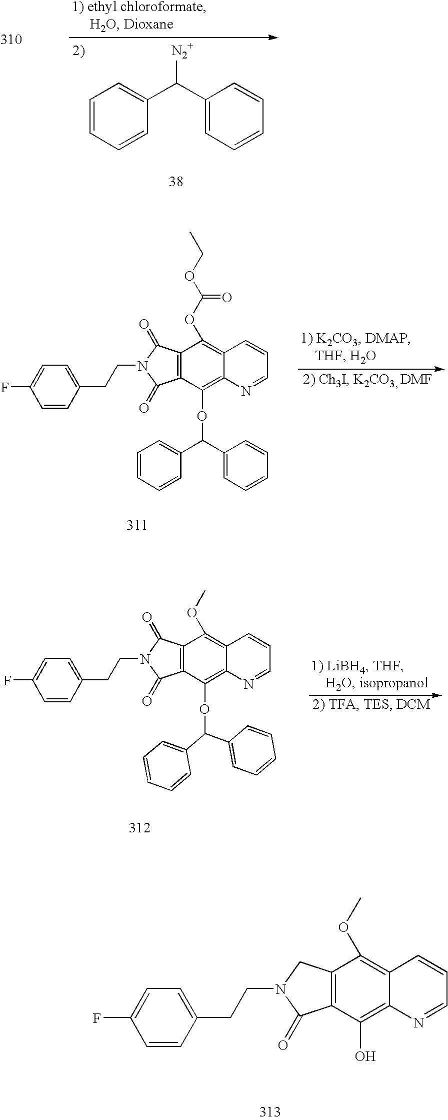

### Example 311

7-[2-(4-Fluoro-phenyl)-ethyl]-5,9-dihydroxy-pyrrolo[3,4-g]quinoline-6,8-dione 310 (250 mg, 0.71 mmol) was dissolved in dioxane (3.6 ml) and H₂O (2.4 ml) and cooled to 0° C. After 1.0 M NaOH (1.42 ml, 1.42 mmol) and ethylchloroformate (84.6 mg, 0.78 mmol) were added, the reaction was stirred at 0° C. for one hour. The reaction mixture was quenched with the addition of acetic acid (0.6 ml) and concentrated in vacuo. The crude mixture was diluted with ethyl acetate and washed once with 5% Citric Acid (aqueous), twice with water, once with brine, dried over magnesium sulfate, and concentrated in vacuo. The resulting residue was dissolved in 1,2-dichloroethane (4.0 ml) and to this was added diphenyl-methanediazonium 38 (252 mg, 1.3 mmol). The reaction mixture was then stirred overnight at room temperature. Following dilution with dichloromethane, the reaction mixture was washed with once with water, once with brine, dried over magnesium sulfate, and concentrated in vacuo. The residue was then purified by silica gel chromatography (1/1—hexane/ethyl acetate) to afford carbonic acid 9-benzhydryloxy-7-[2-(4-fluoro-phenyl)-ethyl]-6,8-dioxo-7,8-dihydro-6H-pyrrolo[3,4-g]quinolin-5-yl ester ethyl ester 311 (251 mg, 0.425 mmol, 65%). ¹H NMR (d-DMSO) δ 9.19 (dd, 1H), 8.52 (dd, 1H), 7.90 (s, 1H), 7.80 (m, 1H), 7.54 (m, 4H), 7.20 (m, 8H), 7.02 (t, 2H), 4.24 (q, 2H), 3.79 (t, 2H), 2.90 (t, 2H), 1.25 (t, 3H). MS: 599.2 (M+23).

### Example 312

To carbonic acid 9-benzhydryloxy-7-[2-(4-fluoro-phenyl)-ethyl]-6,8-dioxo-7,8-dihydro-6H-pyrrolo[3,4-g]quinolin-5-yl ester ethyl ester 311 (140 mg, 0.24 mmol) dissolved in tetrahydrofuran (0.50 ml) and water (0.25 ml) was added potassium carbonate (345.4 mg, 2.5 mmol) and N,N-dimethyl-aminopyridine (DMAP, 29.3 mg, 3.8 mmol). After stirring overnight at room temperature, the reaction mixture was concentrated in vacuo and diluted with ethyl acetate. It was then washed twice with 5% citric Acid (aqueous), twice with water, once with brine, dried over magnesium sulfate, and concentrated in vacuo. The resulting residue was dissolved in dimethylformamide (3.0 ml). To this solution was added potassium carbonate (179 mg, 1.3 mmol) and iodomethane (319 mg, 2.6 mmol). After stirring overnight at room temperature, the reaction mixture was diluted with ethyl acetate. It was then washed twice with 5% citric Acid, twice with water, once with brine, and concentrated in vacuo to afford 9-benzhydryloxy-7-[2-(4-fluoro-phenyl)-ethyl]-5-methoxy-pyrrolo[3,4-g]quinoline-6,8-dione 312 (130 mg, 0.24 mmol, 100%). ¹H NMR (CDCl₃) δ 9.16 (dd, 1H), 8.58 (dd, 1H), 7.82 (s, 1H), 7.74 (m, 1H), 7.55 (m, 4H), 7.20 (m, 8H), 7.0 (t, 2H), 4.04 (s, 3H), 3.87 (t, 2H), 2.91 (t, 2H). MS: 555.2 (M+23).

### Example 313

9-Benzhydryloxy-7-(2,4-dimethoxy-benzyl)-5-methoxy-pyrrolo[3,4-g]quinoline-6,8-dione 312 (130 mg, 0.24 mmol) was dissolved in tetrahydrofuran (1.6 ml), water (0.64 ml), and isopropanol (0.32 ml) and cooled to 0° C. Lithium borohydride (26.6 mg, 1.22 mmol) was then added and the reaction mixture was removed from the ice bath and stirred at room temperature for 2 hours. After quenching with acetic acid (0.12 ml), the reaction mixture was diluted with ethyl acetate. It was then washed with twice with water, once with brine, and concentrated in vacuo. The resulting residue was dissolved in dichloromethane (1.2 ml) and triethylsilane (0.6 ml) and trifluoroacetic acid (3.6 ml). The reaction mixture was then stirred at room temperature for 1 hour. The mixture was then concentrated in vacuo and azeotroped three times with a (1:1) toluene/tetrahydrofuran solution. The resulting residue was triturated three times with a (3:1) hexane/ether mixture and the remaining solid in the filter funnel and reaction flask was dissolved in methanol, combined, and concentrated in vacuo to afford 7-[2-(4-Fluoro-phenyl)-ethyl]-9-hydroxy-5-methoxy-6,7-dihydro-pyrrolo[3,4-g]quinolin-8-one 313 (40 mg, 0.086 mmol, 36%). ¹H NMR (d-DMSO) δ 8.85 (dd, 1H), 8.648 (dd, 1H), 7.65 (m, 1H), 7.28 (t, 2H), 7.06 (t, 2H), 4.60 (s, 2H), 3.95 (s, 3H), 3.68 (t, 2H), 2.95 (t, 2H). ¹⁹F NMR δ 60.0, −75.6. MS: 353.1 (M+1).

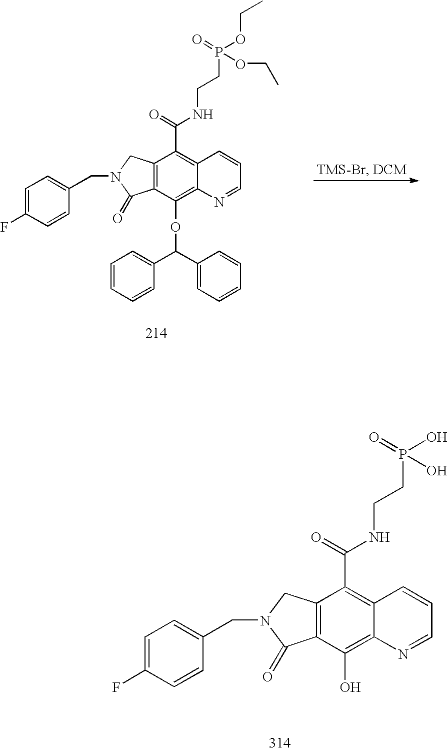

### Example 314

To (2-{[9-benzhydryloxy-7-(4-fluoro-benzyl)-8-oxo-7,8-dihydro-6H-pyrrolo[3,4-g]quinoline-5-carbonyl]-amino}-ethyl)-phosphonic acid diethyl ester 214 (16 mg, 0.023 mmol) dissolved in dichloromethane (0.30 ml) was added trimethylsilylbromide (TMS-Br, 39 mg, 0.25 mmol). After 4 hours of stirring at room temperature, more trimethylsilylbromide (24 mg, 0.16 mmol) was added and the reaction mixture stirred for another 2 hours. The reaction mixture was cooled to 0° C., quenched with methanol (1.0 ml), and concentrated in vacuo. It was then triturated three times (3/1—hexane/ether) and the remaining residue in the flask and filter was dissolved in methanol, combined, and concentrated in vacuo. The residue was dissolved in dimethysulfoxide (0.40 ml), filtered through a glass plug, and purified by reverse-phase preparatory HPLC to provide (2-{[7-(4-fluoro-benzyl)-9-hydroxy-8-oxo-7,8-dihydro-6H-pyrrolo[3,4-g]quinoline-5-carbonyl]-amino}-ethyl)-phosphonic acid 314 (7 mg, 0.012 mmol, 52%). ¹H NMR (CD₃OD) δ 8.96 (d, 1H), 8.77 (d, 1H), 7.78 (m, 1H), 7.42 (m, 2H), 7.10 (t, 2H), 4.80 (s, 2H), 4.63 (s, 2H), 3.72 (m, 2H), 2.16 (m, 2H). ³¹P δ 25.0. ¹⁹F δ −78.0, −116.0. MS: 460.1 (M+1).

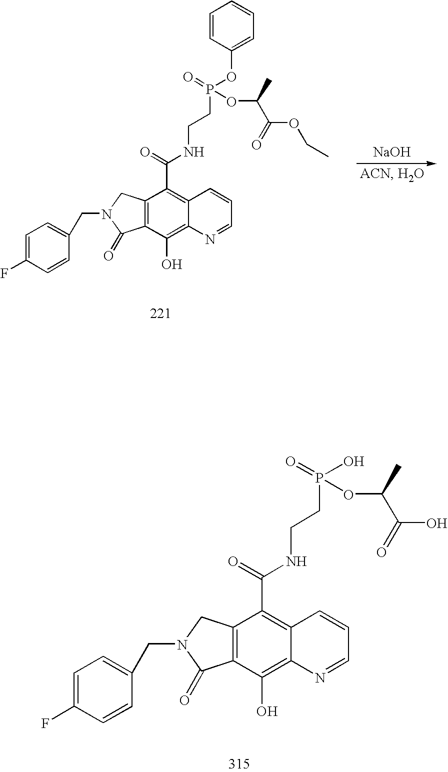

### Example 315

To 2-[(2-{[7-(4-fluoro-benzyl)-9-hydroxy-8-oxo-7,8-dihydro-6H-pyrrolo[3,4-g]quinoline-5-carbonyl]-amino}-ethyl)-phenoxy-phosphinoyloxy]-propionic acid ethyl ester 221 (15 mg, 0.024 mmol) dissolved in acetonitrile (0.10 ml) and water (0.05 ml) was added 1.0 M NaOH (0.072 ml). The reaction mixture was stirred at room temperature for 3 hours, cooled to 0° C., and quenched with 1.0 M HCl (0.1 ml). The mixture was concentrated in vacuo and the resulting residue was redissolved in dimethylsulfoxide, filtered through a glass plug, and purified by reverse phase preparatory HPLC to afford 2-[(2-{[7-(4-fluoro-benzyl)-9-hydroxy-8-oxo-7,8-dihydro-6H-pyrrolo[3,4-g]quinoline-5-carbonyl]-amino}-ethyl)-hydroxy-phosphinoyloxy]-propionic acid 315 (9 mg, 0.014 mmol, 60%). ¹H NMR (CD₃OD) δ 9.0 (d, 1H), 8.80 (d, 1H), 7.80 (m, 1H), 7.42 (M, 2H), 7.10 (t, 2H), 4.80 (d, 2H), 4.62 (s, 2H), 3.75 (m, 2H), 2.20 (m, 2H), 1.46 (d, 3H). ³¹P δ 27.8. ¹⁹F δ −78.0, −118.0. MS: 532.1 (M+1).

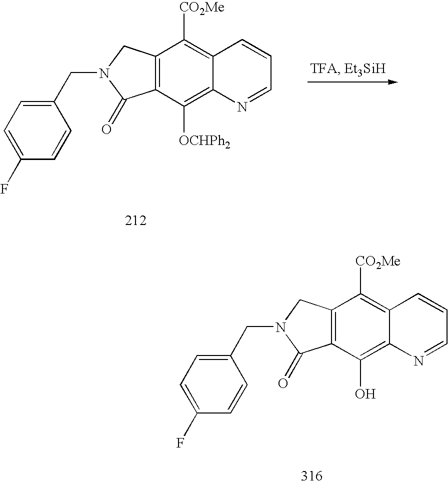

### Example 316

To a solution of 9-benzhydryloxy-7-(4-fluoro-benzyl)-8-oxo-7,8-dihydro-6H-pyrrolo[3,4-g]quinoline-5-carboxylic acid methyl ester 212 (3 mg, 0056 mmol) in dichloromethane (1 mL) were added TFA (0.1 mL) and triethylsilane (0.2 mL). Stirring was continued at the room temperature for 1 hour and the volatiles were evaporated in vacuo. The residue was triturated in Et₂O/hexane to afford 7-(4-fluoro-benzyl)-9-hydroxy-8-oxo-7,8-dihydro-6H-pyrrolo[3,4-g]quinoline-5-carboxylic acid methyl ester 316 (2.0 mg, 100%) as a yellow solid: ¹H NMR (CDCl₃) δ 9.5 (d, 1H), 9.0 (m, 1H), 7.66 (dd, 1H), 7.35 (dd, 2H), 7.0 (t, 2H), 4.8 (s, 2H), 4.7 (s, 2H), 4.0 (s, 3H); MS: 365 (M−1).

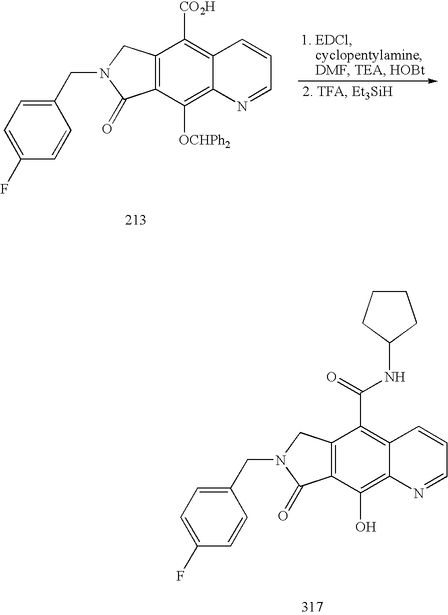

### Example 317

To a solution of 9-benzhydryloxy-7-(4-fluoro-benzyl)-8-oxo-7,8-dihydro-6H-pyrrolo[3,4-g]quinoline-5-carboxylic acid 213 (6 mg, 0.0116 mmol) in DMF (0.5 mL) at the room temperature were added triethylamine (TEA, 5 μL, 0.034 mmol), cyclohexylamine (2.3 μL, 0.022 mmol), 1-(3-dimethylaminopropyl)-3-ethylcarbodiimide hydrochloride (EDCl, 4.4 mg, 0.022 mmol) and 1-hydroxybenzotriazole (HOBt, 2.3 mg, 0.0174 mmol). The solution was stirred under a nitrogen atmosphere for 5 hours and diluted with EtOAc. The organic layer was washed with water, 1N aqueous HCl, saturated aqueous NaHCO₃ and brine, dried over MgSO₄ and concentrated in vacuo. The crude product was chromatographed on a silica gel column eluting with EtOAc/hexane to afford the protected final product, which was treated in dichloromethane (1 mL) with TFA (0.1 mL) and triethylsilane (0.2 mL) at the room temperature for 1 hour. The volatiles were evaporated in vacuo and the residue was triturated in Et₂O/hexane to afford 7-(4-fluoro-benzyl)-9-hydroxy-8-oxo-7,8-dihydro-6H-pyrrolo[3,4-g]quinoline-5-carboxylic acid cyclopentylamide 317 (2.6 mg, 54%) as yellow solid. ¹H NMR (CDCl₃) δ 8.96 (dd, 1H), 8.53 (d, 1H), 7.62 (dd, 1H), 7.27 (m, 2H), 7.04 (t, 2H), 6.34 (m, 1H), 4.63 (s, 2H), 4.48 (m, 3H), 2.2 (m, 2H), 1.50-1.90 (m, 6H); MS: 418 (M−1).

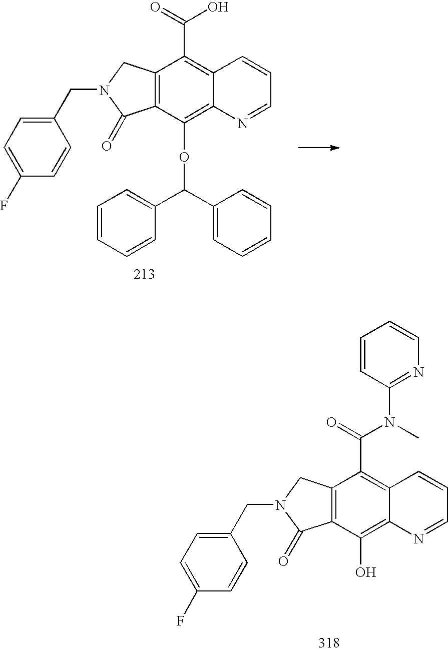

### Example 318

9-Benzhydryloxy-7-(4-fluoro-benzyl)-8-oxo-7,8-dihydro-6H-pyrrolo[3,4-g]quinoline-5-carboxylic acid 213 (0.02 g, 0.0386 mmol) was dissolved in 0.3 mL of dimethylformamide. To this was added 2-methylaminopyridine (0.0079 mL, 0.0772 mmol), diisopropylethylamine (0.027 mL, 0.1544 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Purified by reverse phase HPLC (0.1% TFA, H₂O/ACN) to give 7-(4-fluoro-benzyl)-9-hydroxy-8-oxo-7,8-dihydro-6H-pyrrolo[3,4-g]quinoline-5-carboxylic acid methyl-pyridin-2-yl-amide 318 (0.0017 g, 0.003 mmol, 8%.) ¹H NMR (CDCl₃) δ 9.02 (dd, 1H), 8.50 (d, 1H), 8.18 (d, 1H), 7.65 (dd, 1H), 7.38 (m, 5H), 7.08 (dd, 2H), 4.94 (dd, J=15 Hz, 11 Hz, 2H), 4.49 (d, J=17 Hz, 1H), 4.19 (d, J=17 Hz, 1H), 3.61 (s, 3H.) MS:

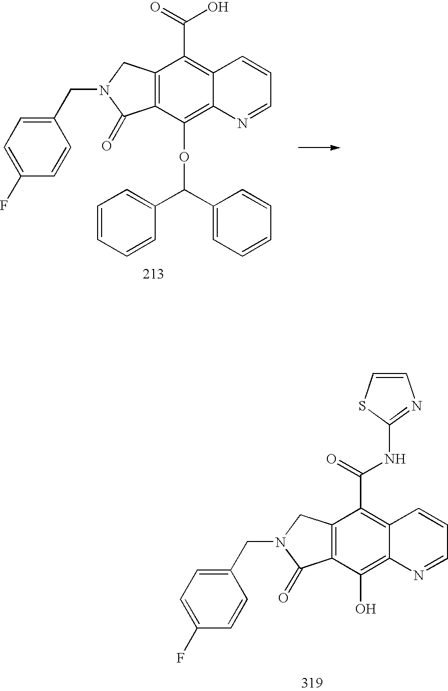

### Example 319

9-Benzhydryloxy-7-(4-fluoro-benzyl)-8-oxo-7,8-dihydro-6H-pyrrolo[3,4-g]quinoline-5-carboxylic acid 213 (0.02 g, 0.0386 mmol) was dissolved in 0.3 mL of dimethylformamide. To this was added 2-aminothiazole (0.0077 mL, 0.0772 mmol), diisopropylethylamine (0.027 mL, 0.1544 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Purified by reverse phase HPLC (0.1% TFA, H₂O/ACN) to give 7-(4-fluoro-benzyl)-9-hydroxy-8-oxo-7,8-dihydro-6H-pyrrolo[3,4-g]quinoline-5-carboxylic acid thiazol-2-ylamide 319 (0.01 g, 0.023 mmol, 60%.) ¹H NMR (CDCl₃) δ 9.02 (dd, 1H), 8.61 (d, 1H), 7.65 (dd, 1H), 7.55 (d, 1H), 7.38 (dd, 2H), 7.21 (d, 1H), 7.07 (dd, 2H), 4.78 (s, 2H), 4.67 (s, 2H.) MS: 435 (M+1).

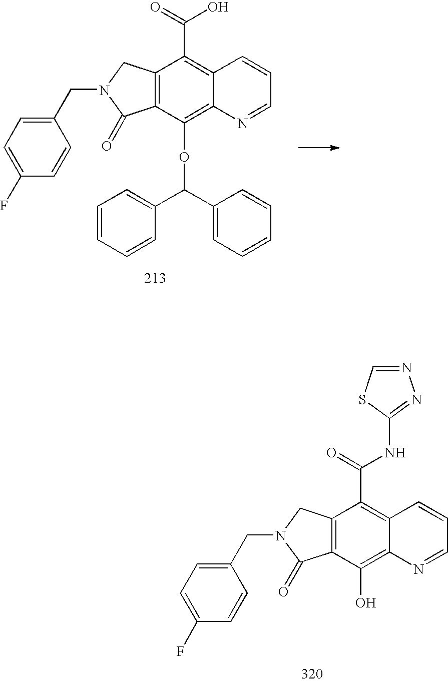

### Example 320

9-Benzhydryloxy-7-(4-fluoro-benzyl)-8-oxo-7,8-dihydro-6H-pyrrolo[3,4-g]quinoline-5-carboxylic acid 213 (0.02 g, 0.0386 mmol) was dissolved in 0.3 mL of dimethylformamide. To this was added 2-amino-1,3,4-thiadiazole (0.0078 mL, 0.0772 mmol), diisopropylethylamine (0.027 mL, 0.1544 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Purified by reverse phase HPLC (0.1% TFA, H₂O/ACN) to give 7-(4-fluoro-benzyl)-9-hydroxy-8-oxo-7,8-dihydro-6H-pyrrolo[3,4-g]quinoline-5-carboxylic acid [1,3,4]thiadiazol-2-ylamide 320 (0.0066 g, 0.015 mmol, 40%.) ¹H NMR (CDCl₃) δ 9.02 (dd, 1H), 8.81 (s, 1H), 8.65 (d, 1H), 7.65 (dd, 1H), 7.38 (dd, 2H), 7.05 (dd, 2H), 4.74 (s, 2H), 4.64 (s, 2H.) MS: 436 (M+1).

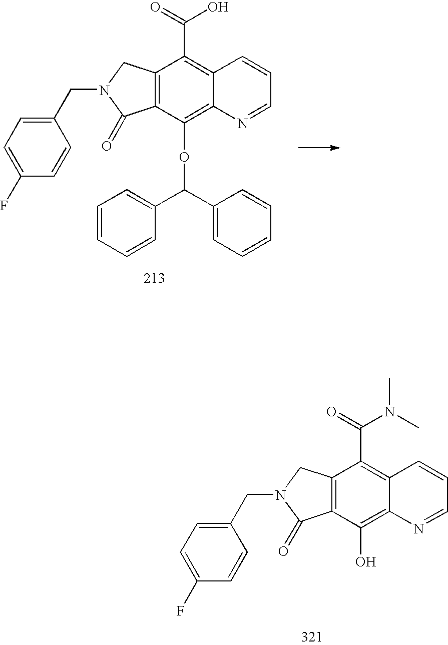

### Example 321

9-Benzhydryloxy-7-(4-fluoro-benzyl)-8-oxo-7,8-dihydro-6H-pyrrolo[3,4-g]quinoline-5-carboxylic acid 213 (0.02 g, 0.0386 mmol) was dissolved in 0.3 mL of dimethylformamide. To this was added dimethylamine (2M in THF) (0.0386 mL, 0.0772 mmol), diisopropylethylamine (0.027 mL, 0.1544 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Purified by reverse phase HPLC (0.1% TFA, H₂O/ACN) to give 7-(4-fluoro-benzyl)-9-hydroxy-8-oxo-7,8-dihydro-6H-pyrrolo[3,4-g]quinoline-5-carboxylic acid dimethylamide 321 (0.014 g, 0.037 mmol, 97%.) ¹H NMR (CDCl₃) δ 9.07 (dd, 1H), 8.18 (d, 1H), 7.65 (dd, 3H), 7.03 (dd, 2H), 4.79 (dd, 2H), 4.53 (d, J=17 Hz, 1H), 4.25 (d, J=17 Hz, 1H), 3.24 (s, 3H), 3.21 (s, 3H.) MS: 380 (M+1).

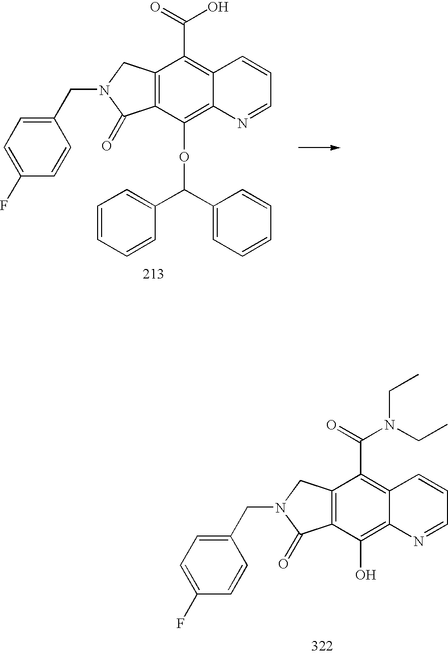

### Example 322

9-Benzhydryloxy-7-(4-fluoro-benzyl)-8-oxo-7,8-dihydro-6H-pyrrolo[3,4-g]quinoline-5-carboxylic acid 213 (0.02 g, 0.0386 mmol) was dissolved in 0.3 mL of dimethylformamide. To this was added diethylamine (0.0056 mL, 0.0772 mmol), diisopropylethylamine (0.027 mL, 0.1544 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Purified by reverse phase HPLC (0.1% TFA, H₂O/ACN) to give 7-(4-fluoro-benzyl)-9-hydroxy-8-oxo-7,8-dihydro-6H-pyrrolo[3,4-g]quinoline-5-carboxylic acid diethylamide 322 (0.0134 g, 0.033 mmol, 86%.) ¹H NMR (CDCl₃) δ 9.07 (dd, 1H), 8.18 (d, 1H), 7.65 (m, 3H), 7.07 (dd, 2H), 4.72 (dd, 2H), 4.56 (d, J=17 Hz, 1H), 4.23 (d, J=17 Hz, 1H), 3.66 (q, 2H), 3.11 (q, 2H), 1.35 (t, 3H), 0.965 (t, 3H.) MS: 408 (M+1).

### Example 323

### HIV Integrase Assay (IC₅₀ Determination)

IC50 is the inhibitory concentration that reduces the strand transfer activity of recombinant integrase by 50%.

HIV Integrase assay was carried out in Reacti-Bind High Binding Capacity Streptavidin coated plates (Pierce # 15502) in 100 μl reactions following the method of Hazuda et al Nucleic Acids Res. (1994) 22:1121-22. The wells of the plate are rinsed once with PBS. Each well is then coated at room temperature for 1 h with 100 μl of 0.14 μM double-stranded donor DNA of Hazuda et al.

After coating, the plate was washed twice with PBS. 3′processing of the donor DNA is started by adding 80 μl of Integrase/buffer mixture (25 mM HEPES, pH 7.3, 12.5 mM DTT, 93.75 mM NaCl, 12.5 mM MgCl₂, 1.25% Glycerol, 0.3125 μM integrase) to each well. 3′-Processing was allowed to proceed for 30 min at 37° C., after which, 10 μl of test compound and 10 μl of 2.5 μM digoxigenin (DIG)-labeled, double-stranded Target DNA, according to Hazuda et al, were added to each well to allow strand transfer to proceed for 30 min at 37° C. The plate was then washed three times with 2×SSC for 5 min and rinsed once with PBS. For detection of integrated product, 100 μl of a 1/2000 dilution of HRP-conjugated anti-DIG antibody (Pierce #31468) were added to each well and incubated for 1 hour. The plate was then washed three times for 5 min each, with 0.05% Tween-20 in PBS. For signal development and amplification, 100 μl of SuperSignal ELISA Femto Substrate (Pierce #37075) were added to each well. Chemiluminescence (in relative light units) was read immediately at 425 nm in the SPECTRAmax GEMINI Microplate Spectrophotometer using the end point mode at 5 sec per well. For IC₅₀ determinations, eight concentrations of test compounds in a 1/2.2 dilution series were used. Certain compounds of the invention had a strand transfer IC₅₀ less than about 10 μM.

### Example 324

### Anti-HIV Assay (EC₅₀ Determination)

EC50 (also commonly referred to as ED50 or IC50) is the effective concentration that inhibits 50% of viral production, 50% of viral infectivity, or 50% of the virus-induced cytopathic effect.

Anti-HIV assay was carried out in 96-well Clear Bottom Black Assay Plate (Costar # 3603) in 100 μl of culture medium, using the CellTiter-Glo™ Reagent (Promega # G7570) for signal detection. MT-2 cells (1.54×10⁴ cells) were infected with wild-type virus at an m.o.i. of about 0.025, and grown in the presence of various drug concentrations (serial 5-fold dilutions) in 100 μl of RPMI medium containing 10% FBS, 2% glutamine, 1% HEPES and 1% penicillin/streptomycin for 5 days. At the end of the incubation period, 100 μl of CellTiter-Glo™ Reagent was added to each well in the Assay Plate and the chemiluminescence (in relative light units) was measured after 10 mins of incubation with the Wallac Victor² 1420 MultiLabel Counter. Certain compounds of the invention had an anti-HIV MT2 EC₅₀ less than about 10 μM.

### Example 325

### Cytotoxicity Assay (CC₅₀ Determination)

For the determination of compound cytotoxicity, the plate and reagents are the same as those of anti-HIV assay. Uninfected MT-2 cells (1.54×10⁴ cells) were grown in the presence of various drug concentrations (serial 3-fold dilutions) in 100 μl of RPMI medium containing 10% FBS, 2% glutamine, 1% HEPES and 1% penicillin/streptomycin for 5 days. At the end of the incubation period, 100 μl of CellTiter-Glo™ Reagent was added to each well in the assay plate and the chemiluminescence (in relative light units) was measured after 10 mins of incubation with the Wallac Victor² 1420 MultiLabel Counter. Certain compounds of the invention had cytotoxicity MT2 CC₅₀ less than about 10 μM.

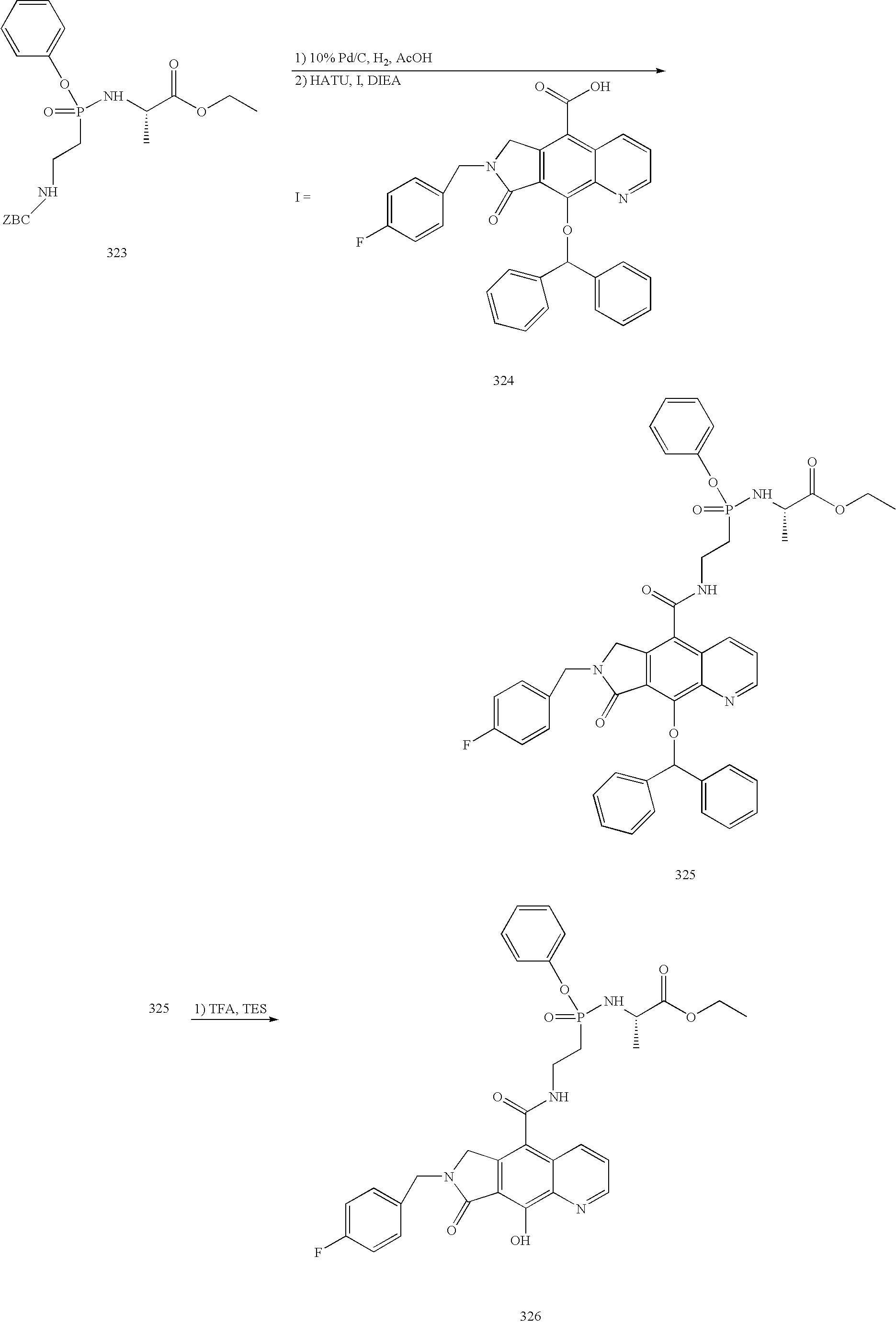

### Example 326

400 mg (0.9 mmol) of 323 dissolved in 3 ml of ethanol and 216 ul of acetic acid was allowed to react with 100 mg of 10% palladium on carbon and H₂ (1 atm) for 30 minutes. The reaction was then filtered through celite and concentrated in vacuo. The residue was then dissolved in DCM, washed once with saturated Na₂CO₃, and concentrated in vacuo. The residue was then added to I (155 mg, 0.3 mmol) which had been reacting with HATU (220 mg, 0.6 mmol) in DMF for 10 minutes. This mixture was then treated with DIEA (155.1 mg, 1.2 mmol) and stirred at room temperature for one hour. The mixture was diluted with ethyl acetate, washed with saturated NaHCO₃ (twice), water (twice) and brine (twice), dried (Na₂SO₄), and concentrated. The residue was purified by silica gel chromatography (2% methanol in ethyl acetate) to afford 2 (213 mg, 80% yield)

325 (0.1 g, 0.12 mmol) in methylene chloride (0.9 mL) was treated with trifluoracetic acid (0.045 mL) and triethylsilane (0.075 mL). The reaction mixture was stirred at room temperature under an inert atmosphere for 40 minutes. The volatiles were removed in vacuo with toluene. The product was triturated (1/3—diethyl ether/hexane with sonicaton to afford 326 (75 mg, 83% yield) as the mono-triflouroacetate salt. ¹H NMR (CD₃OD) δ 8.89 (bs, 1H), 8.70 (bs, 0.5H), 8.65 (d, 1.0), 7.67 (bs, 1H), 7.35 (m, 4H), 7.22 (t, 3H), 7.10 (t, 3H), 4.74 (bs, 2H), 4.59 (bs, 2H), 4.10 (m, 2H), 3.97 (m, 1H), 3.84 (bs, 2H), 2.39 (m, 2H). ³¹P NMR (CDCl₃) δ 30.10, 29.16. MS=635.1 (M+1).

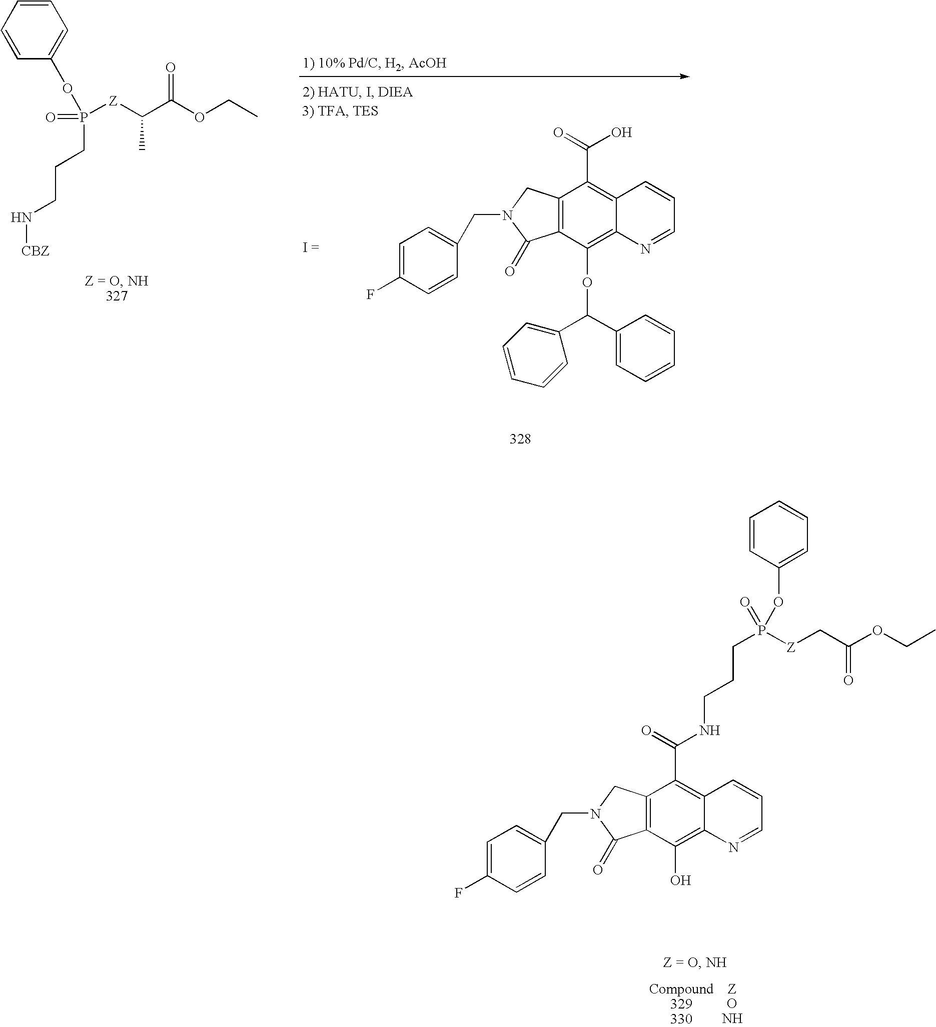

### Example 327

The syntheses of 329 and 330 from starting materials of type 327 were carried out in a manner similar to that stated previously. 329 ¹H NMR (CD₃OD) δ 8.90 (bs, 1H), 8.57 (b, 1H), 7.70 (b, 1H), 7.37 (m, 4H), 7.24 (t, 3H), 7.10 (t, 3H), 4.73 (b, 2H), 4.57 (b, 2H), 4.15 (q, 2H), 3.59 (b, 2H), 2.13 (m, 4H), 1.53 (d, 2H), 1.39 (d, 2H), 1.22 (q, 3H). ³¹P NMR (CDCl₃) δ 30.75, 29.47. MS: 651 (M+1). 330 ¹H NMR (CD₃OD) δ 8.90 (bs, 1H), 8.75 (bs, 0.5H), 8.60 (bs, 1H), 7.75 (m, 1H), 7.37 (m, 4H), 7.24 (t, 3H), 7.10 (t, 3H), 4.80 (bs, 2H), 4.60 (bs, 2H), 4.10 (q, 2H), 3.95 (m, 1H), 3.58 (bs, 2H), 2.10 (m, 2H), 1.28 (d, 2H), 1.20 (m, 4H). ³¹P NMR (CDCl₃) δ 34.18, 33.40. MS: 649 (M+1).

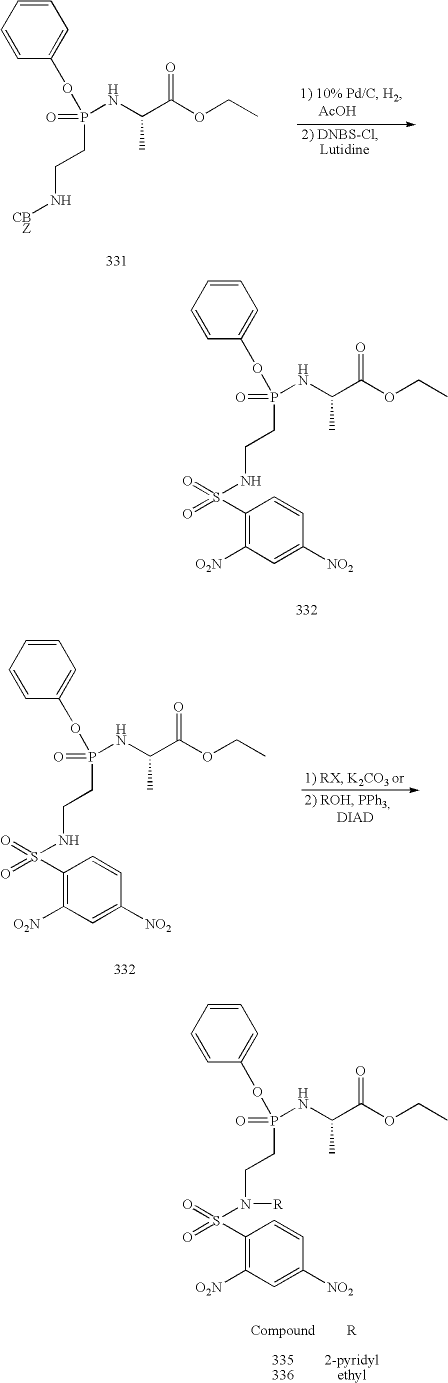

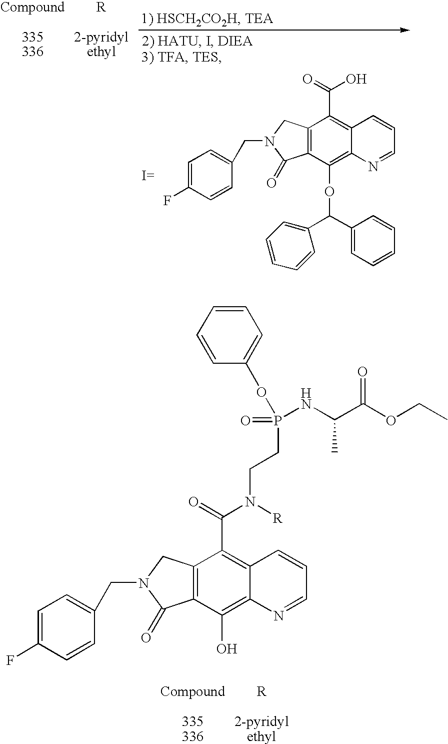

### Example 328

990 mg (2.3 mmol) of 331 dissolved in 10 ml of ethanol and 0.3 ml of acetic acid was treated with 300 mg of 10% palladium on carbon and placed under a hydrogen balloon. After 1 hour, the reaction was filtered through celite and concentrated in vacuo. The residue was then partitioned between DCM and saturated Na₂CO₃. The organic layer was then collected and concentrated in vacuo. The residue was then dissolved in DCM and lutidine (0.801 ml, 6.9 mmol). To this reaction mixture was added 1.8 g (6.9 mmol) of Dinitrobenzenesulfonyl chloride. After stirring for 30 minutes at room temperature, the reaction was quenched with 1.2 ml of acetic acid and concentrated in vacuo. The residue was then diluted with ethyl acetate, washed once with saturated NH₄Cl, once with water, dried over magnesium sulfate, and concentrated in vacuo. The residue was then purified by silica gel chromatography (neat ethyl acetate) to give 795 mg (52% yield) of 332.

332 was then alkylated either by treatment with an alkyl halide in the presence of K₂CO₃ or by treatment with an alkyl alcohol in the presence of PPh₃ and DIAD. For alkylation by treatment with and alkyl halide, the following procedure was followed: 2 (80 mg, 0.19 mmol) dissolved in DMF was allowed to react with iodoethane and K₂CO₃ for 1 hour. The reaction was then diluted with ethyl acetate and washed once with saturated ammonium chloride, once with 2.5% aqueous LiCl, once with water, once with brine, dried over Mg₂SO₄, and concentrated in vacuo. The residue was then purified by silica gel chromatography (neat ethyl acetate) to afford 60 mg (52% yield) of 333.

For alkylation by treatment with an alkyl alcohol, the following procedure was followed: 2 (100 mg, 0.19 mmol) dissolved in DCM was allowed to react with 2-pyridyl-carbinol (62.2 mg, 0.57 mmol), PPh₃ (150 mg, 0.57 mmol), and DIAD (115.2 mg, 0.57 mmol) for one hour at room temperature. The reaction was then concentrated and purified by silica gel chromatography (neat ethyl acetate) to afford 94 mg (80% yield) of 334.

Compounds 333 and 334 were then converted to compounds 336 and 335 respectively using the following representative procedure: 334 (47 mg, 0.08 mmol) dissolved in DCM was allowed to react with mercaptoacetic acid (14,7 mg, 0.16 mmol) and TEA (16.2 mg, 0.16 mmol) for 30 minutes. The reaction was then diluted with DCM, washed twice with saturated Na₂CO₃, and concentrated in vacuo. Amide bond formation between the crude residue and I was then performed in a similar manner as stated previously to give 335. 335 ¹H NMR (CD₃OD) δ 8.91 (m, 1H), 8.52 (m, 1H), 7.95 (m, 1H), 7.71 (m, 2H), 7.54 (m, 2H), 7.39 (m, 4H), 7.24 (m, 3H), 7.12 (m, 3H), 6.88 (m, 1H), 4.71 (m, 1H), 4.52 (m, 4H), 4.01 (m, 2H), 3.98 (m, 1H), 3.64 (m, 2H), 2.30 (m, 2H), 1.32 (m, 6H). ³¹P NMR (CDCl₃) δ 31.363, 31.132, 30.483, 30.237, 29.112, 29.091, 28.598, 28.428. MS: 726 (M+1). 336 ¹H NMR (CD₃OD) δ 8.96 (bs, 1H), 8.30 (m, 1H), 7.74 (b, 1H), 7.38 (m, 4H), 7.24 (m, 3H), 7.10 (m, 3H), 6.83 (m, 1H), 4.69 (t, 1H), 4.43 (b, 1H), 4.07 (m, 4H), 3.65 (m, 2H), 3.20 (m, 2H), 2.28 (m, 2H), 1.17 (m, 10H). ³¹P NMR (CDCl₃) δ 31.06, 30.10, 28.95, 28.14. MS: 663 (M+1).

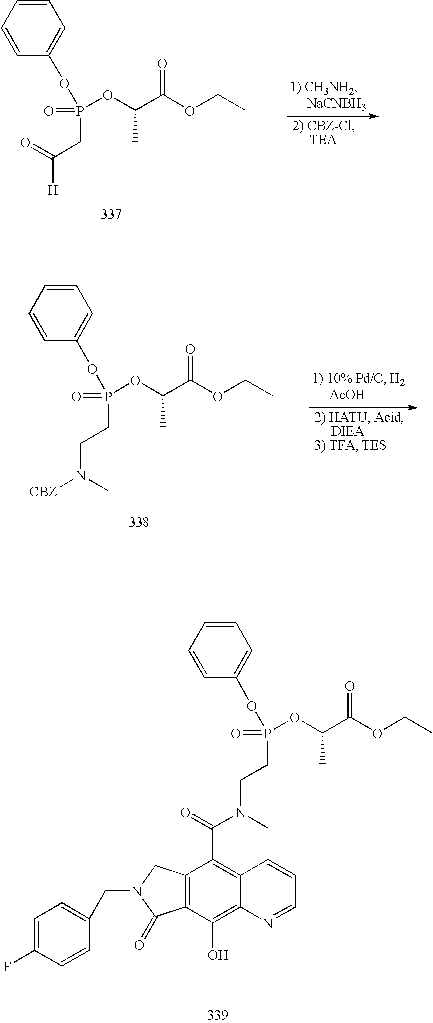

### Example 329

337 (200 mg, 0.67 mmol) dissolved in DMF was allowed to react with acetic acid (0.264 ml, 4.5 mmol) and 2.0 M CH₃NH₂ in THF (2.0 ml, 4.0 mmol) at room temperature for 2 hours. TFA (0.31 ml, 4.5 mmol) and NaCNBH₃ (140 mg, 2.2 mmol) were then added and the reaction was stirred for another hour. The reaction was then diluted with ethyl acetate, washed twice with saturated sodium carbonate, dried over magnesium sulfate, and concentrated in vacuo.

The crude residue was then diluted with DCM and allowed to react with TEA (0.6 ml, 4.3 mmol) and CBZ-Cl (0.366 ml, 2.6 mmol) for 12 hours at room temperature. The reaction was then concentrated in vacuo, diluted with ethyl acetate, washed once with 5% citric acid (aqueous), once with water, once with brine, dried over magnesium sulfate, and concentrated in vacuo. The residue was then purified by silica gel chromatography (neat ethyl acetate) to afford 7 (168.8 mg, 56% yield).

338 was then converted to 339 in a manner similar to that stated previously. ¹H NMR (CD₃OD) δ 8.91 (m, 1H), 8.31 (m, 1H), 7.68 (m, 1H), 7.36 (m, 4H), 7.23 (m, 3H), 7.07 (m, 3H), 6.81 (m, 1H), 4.94 (m, 1H), 4.79 (m, 1H), 4.63 (m, 3H), 4.41 (m, 2H), 4.17 (m, 2H), 3.96 (m, 1H), 3.55 (m, 1H), 3.17 (s, 1.5H), 2.84 (s, 1.5H), 2.59 (m, 1H), 1.53 (m, 1H), 1.37 (m, 1H), 1.2 (m, 4H), 1.03 (d, 1H). ³¹P NMR (CD₃OD) δ 27.70, 27.65, 25.90, 25.75. MS: 650 (M+1).

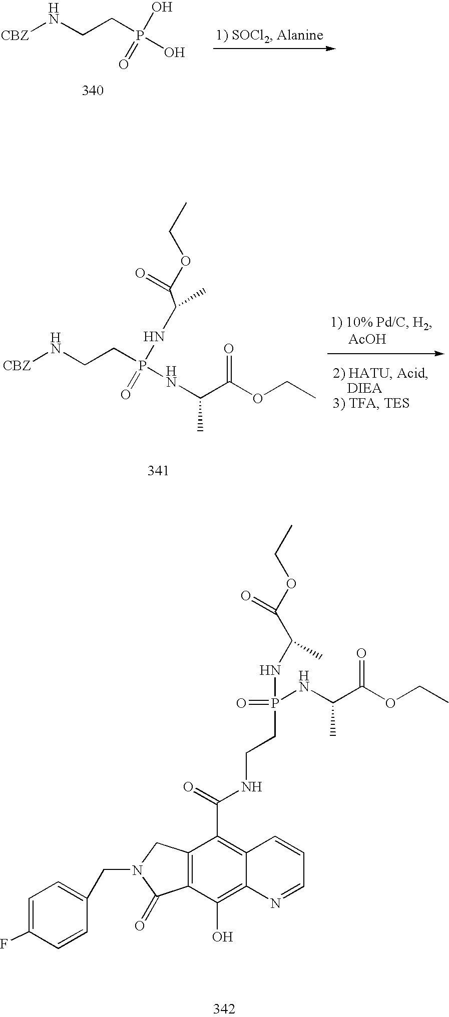

340 (200 mg, 0.73 mmol) dissolved in toluene was allowed to react with thionyl chloride (348 mg, 2.93 mmol) for one hour at 65 C. The mixture was then concentrated in vacuo and azeotroped three times with toluene. The residue was then dissolved in DCM, cooled to 0 C, and allowed to react with L-Alanine ethyl ester (432 mg, 3.66 mmol) for one hour. The reaction was then quenched by addition of acetic acid (0.6 ml) and concecntrated in vacuo. The residue was then diluted with ethyl acetate, washed once with water, once with brine, dried over magnesium sulfate, and concentrated in vacuo. The residue was then purified by silica gel chromatography (5% methanol in ethyl acetate) to afford 341 (103 mg, 30% yield). 341 was then converted into 342 in a manner similar to that stated previously. ¹H NMR (CD₃OD) δ 8.88 (d, 1H), 8.61 (t, 1H) 8.55 (d, 2H), 7.68 (m, 1H), 7.36 (m, 2H), 7.05 (t, 2H), 4.73 (s, 2H), 4.54 (s, 2H), 4.26 (q, 1H), 4.12 (q, 2H), 4.03 (m, 2H), 3.90 (m, 2H), 3.47 (bs, 2H), 1.84 b(m, 2H), 1.49 (d, 1H), 1.33 (t, 7H), 1.21 (m, 7H). ³¹P NMR (CD₃OD) δ 31.6. MS: 672 (M+1).

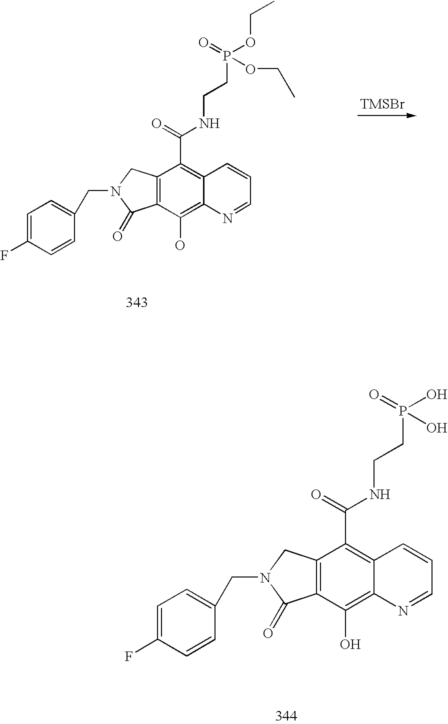

### Example 330

Following the procedure in Example 165, compound 343 was converted to compound 344. ¹H NMR (CD₃OD) δ 8.96 (d, 1H), 8.76 (d, 1H), 7.79 (m, 1H), 7.42 (q, 2H), 7.11 (t, 2H), 4.79 (s, 2H), 4.61 (s, 2H), 3.71 (dt, 2H), 2.13 (dt, 2H). ³¹P NMR (CD₃OD) δ 25.834. MS: 460 (M+1).

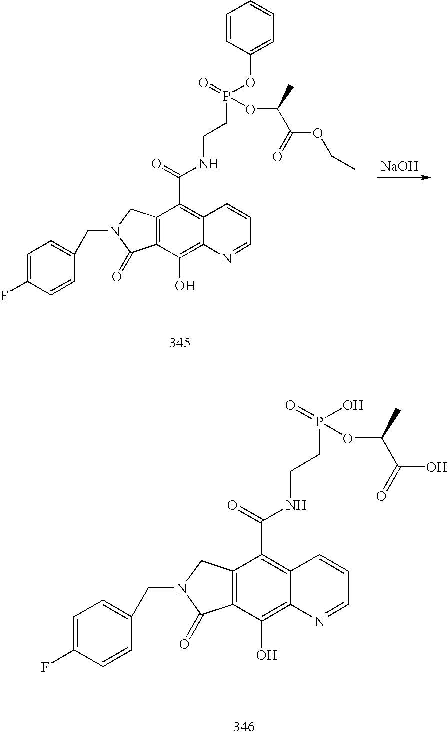

### Example 331

Following the procedure in Example 222, compound 345 was converted to compound 346. ¹H NMR (CD₃OD) δ 8.96 (d, 1H), 8.83 (d, 1H), 7.81 (m, 1H), 7.42 (q, 2H), 7.10 (t, 2H), 4.79 (d, 2H), 4.63 (s. 2H), 3.77 (m, 2H), 2.23 (dt, 2H), 1.49 (d, 3H), ³¹P NMR (CD₃OD) δ 27.588. MS: 532 (M+1).

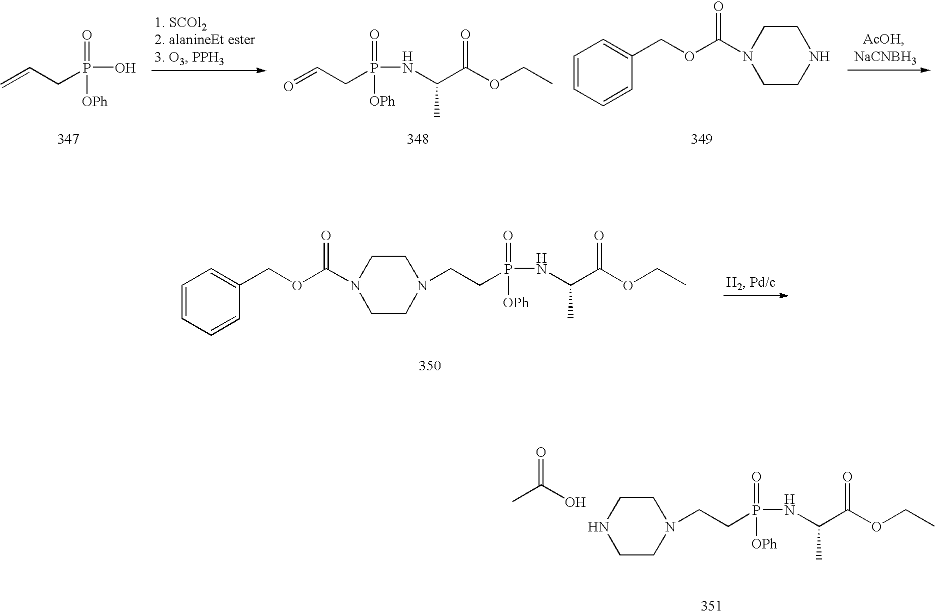

### Example 332

348: A solution of 347 (1.17 g, 5.9 mmol) in toluene (7.4 mL) was treated with thionyl chloride (1.29 mL, 17.7 mmol) and N,N-Dimethylformamide (DMF) (0.040 mL).

The reaction mixture was heated to 65° C. under nitrogen atmosphere and stirred for 2 hours at which point the reaction was complete as shown by ³¹P NMR (CDCl₃) δ 35.8. The reaction mixture was concentrated as such to afford the intermediate mono-chloridate as an oil which was immediately dissolved in methylene chloride (19.7 mL) and cooled to −20° C. L-Alanine ethyl ester hydrochloride was partitioned between methylene chloride and saturated Na₂CO₃. The organic phase was dried (NaSO₄), filtered then concentrated in vacuo to afford the freed base L-Alanine ethyl ester (2.0 g, 17.7 mmol) which was added to the reaction mixture. The mixture was stirred at −20° C. under nitrogen atmosphere for 1 hour and then was concentrated in vacuo. The residue was purified by chromatography on silica gel (2/1—ethyl acetate/hexane) to afford the desired allyl phosphonate mono-amidate intermediate (0.92 g, 52%) as an oil. To a solution of the allyl phosphonate mono-amidate (0.92 g, 3.1 mmol) dissolved in methylene chloride 10.2 mL) cooled to −78° C. was bubbled ozone. After the reaction was saturated and the solution turned a blue color, oxygen was bubbled to remove excess ozone, triphenyl phosphine (1.21 g, 4.61 mmol) was added and the reaction mixture stirred for 1 hour while warming to room temperature. The mixture was concentrated in vacuo without further purification to afford a mixture of the aldehyde product 348 and triphenyl phosphine oxide (2.5 g, 100%).

350: To a solution of the crude aldehyde 348 (0.91 g, 3.1 mmol) and Benzyl 1-piperazine-1-carboxylate 349 (0.740 g, 3.36 mmol) dissolved in ethanol (30.6 mL) was added 4 angstrom molecular sieves (0.300 g) and acetic acid (0.699 mL, 12.22 mmol). The reaction mixture was stirred at room temperature under nitrogen atmosphere for 1.5 hours then sodium cyanoborohydride (0.387 g, 6.15 mmol) was added. The reaction mixture stirred at room temperature for 1 hour and was concentrated in vacuo then redissolved in ethyl acetate. The mixture was washed with saturated NaHCO₃ and brine, dried (NaSO₄), filtered and concentrated. The residue was purified by chromatography on silica gel (2/98—methanol/ethyl acetate) to afford the desired product 350 (0.759 g, 49%) as an oil.

351: To a solution of the phosphonate 350 (0.759 g, 1.5 mmol) dissolved in ethanol (15.0 mL) was added palladium (on carbon). The reaction was purged under a vacuum then submitted to hydrogen gas (via balloon attached to the reaction vessel). After several purges between gas and vacuum the reaction mixture was stirred at room temperature for 2 hours. The mixture was filtered with celite and concentrated in vacuo to afford the amine 351 (0.700 g, 100%) as an acetate salt with a 1:1.3 mixture of diastereomers without further purification: ¹H NMR (CDCl₃) δ 7.4-7.1 (m, 10H), 4.2-4.0 (m, 3H), 3.3-3.1 (m, 4H), 2.9-2.6 (m, 6H), 2.3-2.0 (m, 2H), 1.4 & 1.3 (d, 3H), 1.25 (t, 3H); ³¹P NMR (CDCl₃) δ 30.82, 30.33; MS: 370 (M+1).

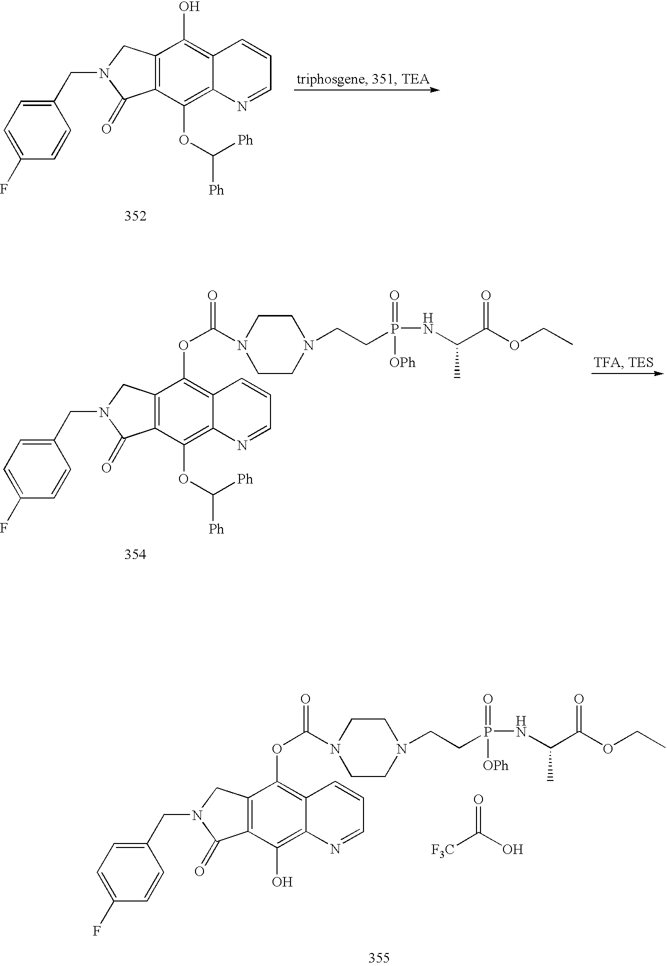

### Example 333

354: A solution of the phenol intermediate 352 (0.115 mmol) in methylene chloride (1.2 mL) was treated with triethylamine (0.065 mL, 0.461 mmol) and cat. 4-dimethylaminopyridine. The reaction mixture was cooled to 0° C. then triphosgene (0.068 g, 0.23 mmol) in a 1M solution of methylene chloride was added. The mixture stirred at room temperature under nitrogen atmosphere for 1 hour, then amine 351 (0.150 g, 0.346 mmol) in a 1M solution of methylene chloride treated with triethylamine (0.065 mL, 0.461 mmol) was added to the reaction, and the mixture was stirred overnight. The mixture was partitioned between methylene chloride and water. The organic phase was washed with brine (twice), dried (MgSO₄), filtered and concentrated in vacuo. The residue was purified by silica gel chromatography (2/98—methanol/ethyl acetate) to afford the product 354 (0.030 g, 30%).

355: A solution of the phosphonate 354 (0.020 g, 0.025 mmol) in methylene chloride (0.5 mL) was treated with trifluoracetic acid (0.1 mL) and triethylsilane (0.2 mL). The reaction mixture was stirred at room temperature under an inert atmosphere for 20 minutes. The volatiles were removed in vacuo with toluene. The solid was triturated in diethyl ether/hexane then purified by reversed phase HPLC to afford the desired product 355 (0.014 g, 80%) as a TFA salt with a 1:1.6 mixture of diastereomers: ¹H NMR (CDCl₃) δ 9.0 (dd, 1H), 8.15 (dd, 1H), 7.6 (dd, 1H), 7.35 (m, 2H), 7.4-7.1 (m, 5H), 7.1 (t, 2H), 4.75 (s, 2H), 4.35 (s, 2H), 4.3-3.2 (m, 15H), 2.7-2.4 (m, 2H), 1.4-1.2 (m, 6H); ³¹P NMR (CDCl₃) δ 26.01, 25.38; MS: 720 (M+1).

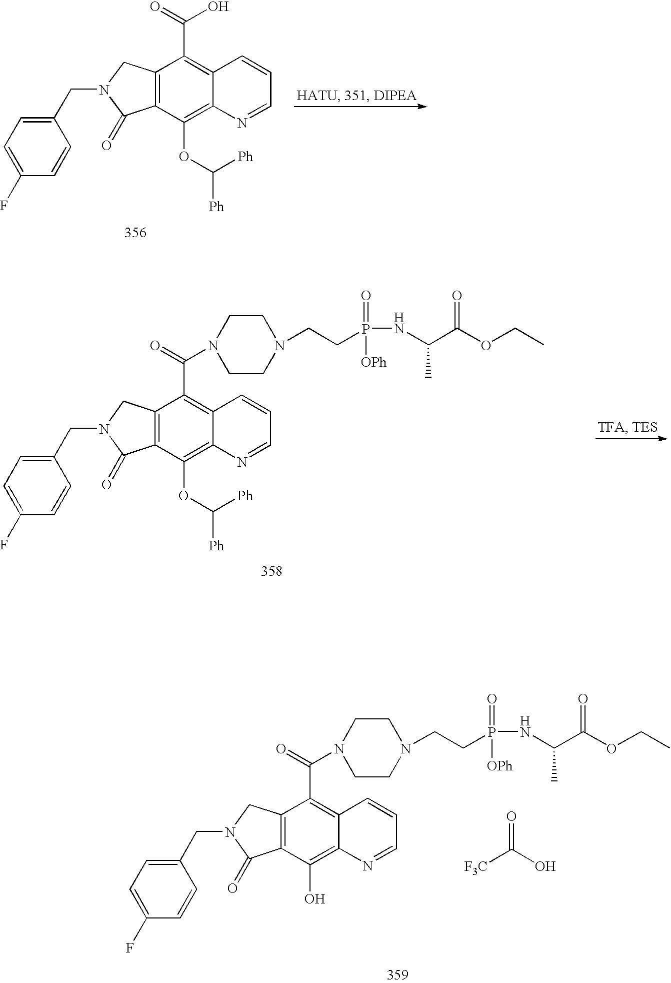

### Example 334

358: The acetate salt 351 was partitioned between methylene chloride and saturated Na₂CO₃. The organic phase was dried (NaSO₄), filtered then concentrated in vacuo to afford the freed base amine (0.260 g, 0.703 mmol) which was dissolved in DMF (1 mL) and treated with diisopropylethylamine (0.20 mL, 1.12 mmol). This mixture was added to a solution of carboxylic acid 356 (0.145 g, 0.281 mmol) in DMF (1.15 mL) that had been stirred with HATU (0.214 g, 0.562 mmol). The reaction mixture was stirred overnight then diluted with ethyl acetate, washed with saturated NH₄Cl, brine (twice), and aqueous LiCl (twice), dried (NaSO₄), filtered and concentrated. The residue was purified by chromatography on silica gel (5/95—methanol/ethyl acetate) to afford the desired product 358 (0.160 g, 65%) as a solid.

359: This compound is synthesized in a similar fashion as compound 355 without need for purification by reversed phase HPLC to afford the desired product 359 (100%) as a TFA salt with a 1:1.5 mixture of diastereomers: ¹H NMR (CDCl₃) δ 9.0 (dd, 1H), 8.15 (dd, 1H), 7.65 (dd, 1H), 7.35 (m, 2H), 7.4-7.1 (m, 5H), 7.05 (t, 2H), 4.9 (m, 1H), 4.5 (s, 2H), 4.3-3.2 (m, 12H), 3.0 (m, 2H), 2.6-2.3 (m, 2H), 1.4-1.2 (m, 6H); ³¹P NMR (CDCl₃) δ 26.21, 25.56; MS: 704 (M+1).

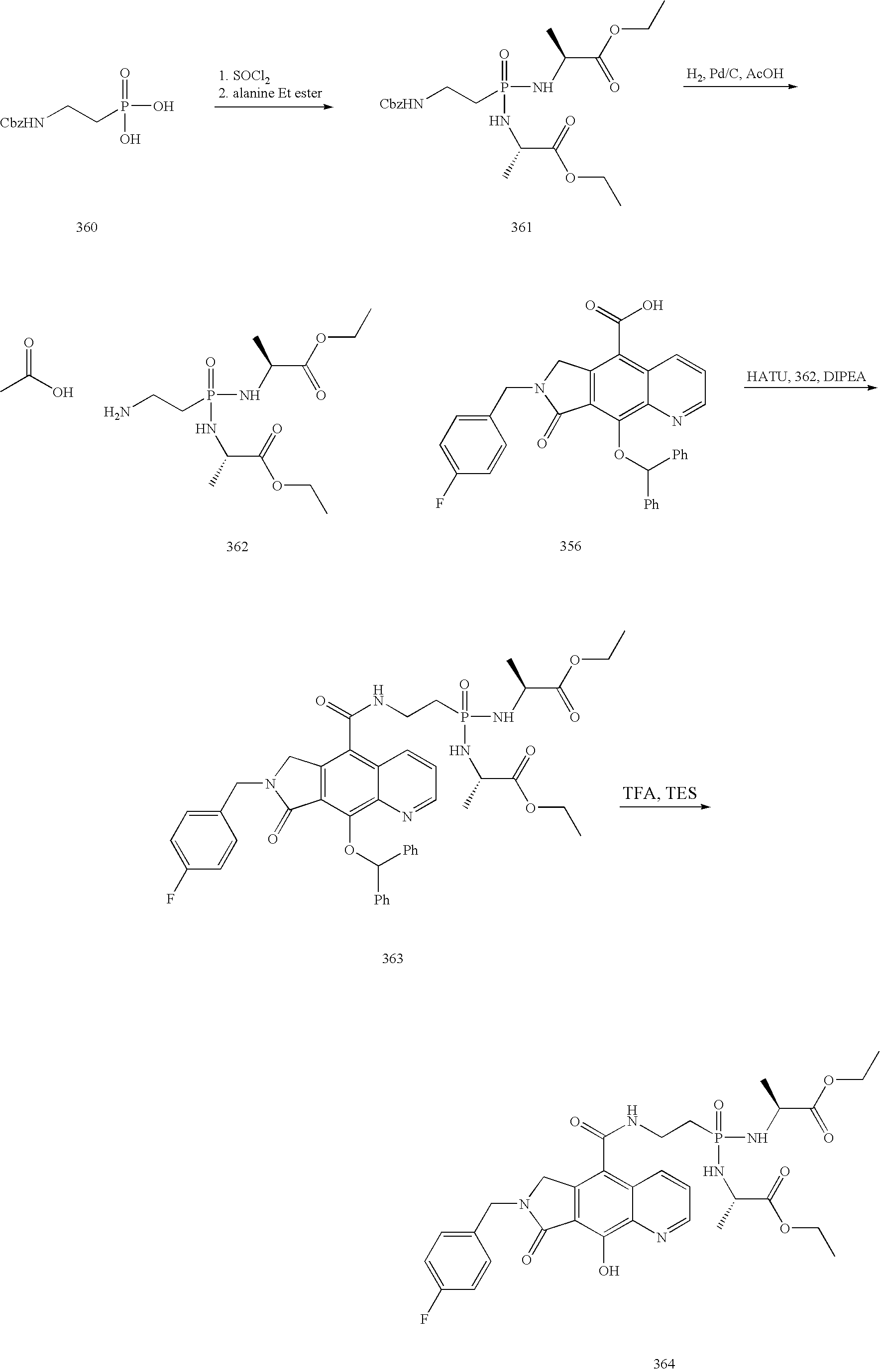

### Example 335

361: A solution of 360 (1.05 g, 4.05 mmol) in toluene (20.3 mL) was treated with thionyl chloride (1.18 mL, 16.2 mmol) and DMF (0.040 mL). The reaction mixture was heated to 65° C. under nitrogen atmosphere and stirred for 3 hours at which point the reaction was complete as shown by ³¹P NMR (CDCl₃) δ 46.5. The reaction mixture was concentrated as such to afford the intermediate mono-chloridate as an oil which was immediately dissolved in methylene chloride (13.5 mL) and cooled to −20° C. L-Alanine ethyl ester hydrochloride was partitioned between methylene chloride and saturated Na₂CO₃. The organic phase was dried (NaSO₄), filtered then concentrated in vacuo to afford the freed base L-Alanine ethyl ester (2.37 g, 20.3 mmol) which was added to the reaction mixture. The mixture was stirred at −20° C. under nitrogen atmosphere for 1 hour and then was concentrated in vacuo. The residue was purified by chromatography on silica gel (3/97—methanol/ethyl acetate) to afford the desired bis-amidate intermediate (1.03 g, 56%) as an oil.

362: The compound was synthesized in a similar fashion as amine 351 to afford the desired amine 362 (100%) as an acetate salt without further purification: ¹H NMR (CDCl₃) δ 4.2-4.0 (m, 3H), 4.0 (m, 2H), 3.3 (m, 2H), 2.4-1.9 (m, 2H), 1.4 (m, 6H), 1.3 (t, 6H); ³¹P NMR (CDCl₃) δ 27.30; MS: 324 (M+1).

363: The compound was made in a similar fashion as compound 358 to afford phosphonate 363.

364: A solution of the phosphonate 363 (0.135 g, 0.164 mmol) in methylene chloride (0.550 mL) was treated with trifluoracetic acid (0.063 mL, 0.82 mmol) and triethylsilane (0.052 mL, 0.328 mmol). The reaction mixture was stirred at room temperature under an inert atmosphere for 20 minutes. The volatiles were removed in vacuo with toluene. The solid was triturated in diethyl ether/hexane then purified by reversed phase HPLC in neutral conditions to afford the desired product 364 (0.014 g, 50%): ¹H NMR (CDCl₃) δ 8.8 (dd, 1H), 8.6 (dd, 1H), 7.9 (dd, 1H), 7.5 (m, 1H), 7.3 (m, 2H), 7.0 (t, 2H), 4.6 (s, 2H), 4.5 (s, 2H), 4.2-3.8 (m, 8H), 2.15 (m, 2H), 1.4 (m, 6H), 1.25 (m, 6H); ³¹P NMR (CDCl₃) δ 28.33; MS: 658 (M+1).

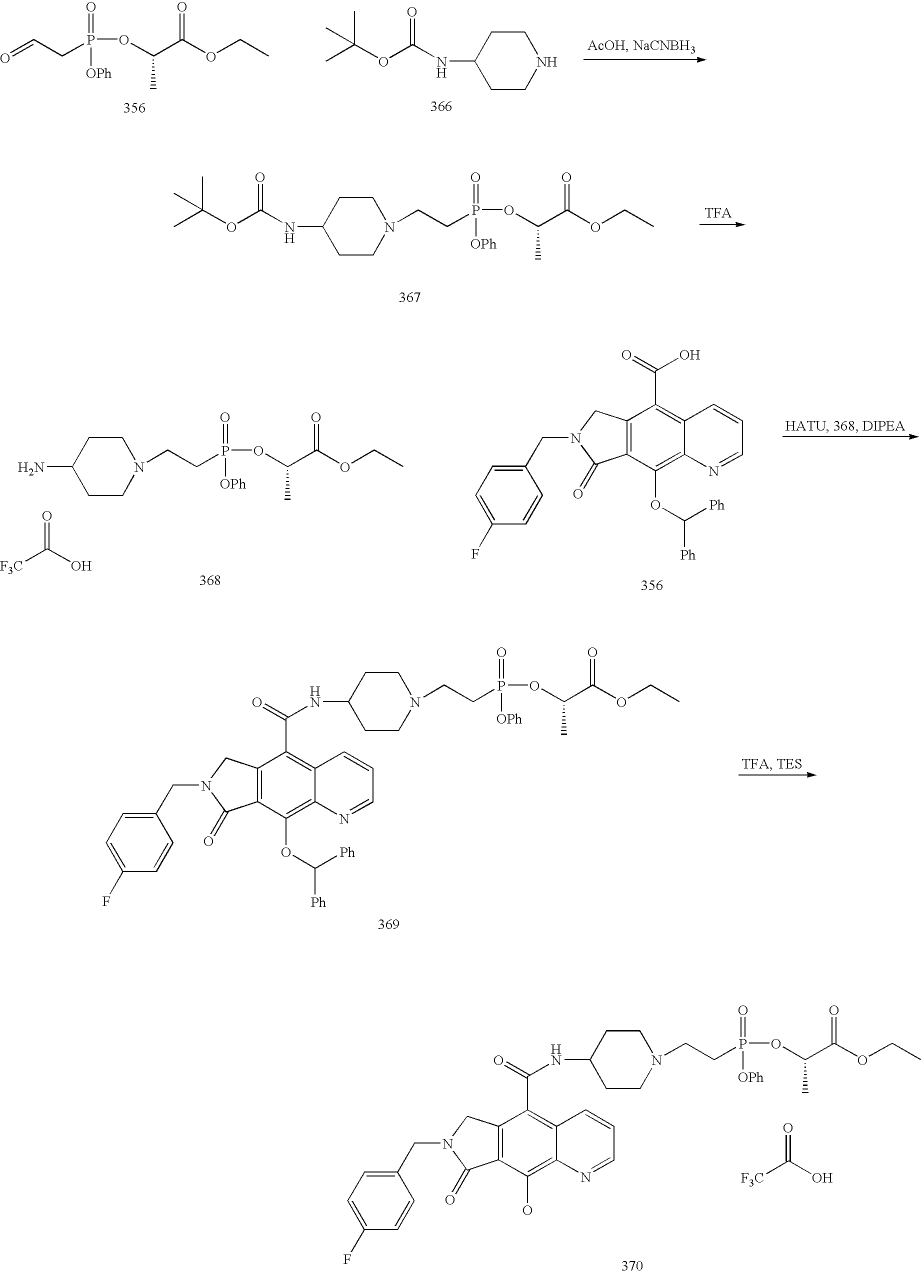

### Example 336

367: To a solution of aldehyde 365 as a 1:1 mixture of DMSO (0.250 g, 0.667 mmol) and 4-BOC aminopiperidine 366 (0.147 g, 0.733 mmol) dissolved in ethanol (6.67 mL) was added 4 angstrom molecular sieves (0.300 g) and acetic acid (0.699 mL, 12.22 mmol). The reaction mixture was stirred under nitrogen atmosphere at room temperature for 1.5 hours then sodium cyanoborohydride (0.387 g, 6.15 mmol) was added. The reaction mixture stirred at room temperature overnight then concentrated in vacuo. The residue was redissolved in ethyl acetate then washed with saturated NaHCO₃ and brine, dried (NaSO₄), filtered and concentrated. The residue was purified by chromatography on silica gel (2/98—methanol/ethyl acetate) to afford the desired product 367 (0.173 g, 54%) as an oil.

368: A solution of the phosphonate 367 (0.173 g, 0.357 mmol) in methylene chloride (2.4 mL) was treated with trifluoracetic acid (0.275 mL, 3.57 mmol). The reaction mixture was stirred at room temperature under an inert atmosphere overnight. The volatiles were removed in vacuo with toluene to afford the free piperazine linker phosphonate 368 (0.190 g, 100%) as a TFA salt.

369: A solution of the amine 368 (0.105 g, 0.213 mmol) in DMF (0.5 mL) was treated with diisopropylamine (0.075 mL, 0.426 mmol). This mixture was added to a solution of carboxylic acid 356 (0.0275 g, 0.053 mmol) in DMF (0.53 mL) that had been stirred with HATU (0.040 g, 0.106 mmol). The reaction mixture was stirred overnight then diluted with ethyl acetate, washed with saturated NH₄Cl, brine (twice), and aqueous LiCl (twice), dried (NaSO₄), filtered and concentrated. The residue was purified by chromatography on silica gel (5/95—methanol/methylene chloride) to afford the desired product 369 (0.044 g) approximately 75% pure.

370: This compound is synthesized in a similar fashion as compound 355 to afford the desired product (70%) as a TFA salt with a 1.2:1 mixture of diastereomers: ¹H NMR (CD₃OD) δ 8.9 (dd, 1H), 8.55 (dd, 1H), 7.7 (dd, 1H), 7.35 (m, 2H), 7.5-7.0 (m, 9H), 5.0 (m, 1H), 4.8-4.4 (m, 2H), 4.3-4.1 (m, 2H), 3.9-3.5 (m, 4H), 2.9-2.6 (m, 2H), 2.5-2.2 (m, 2H), 1.9 (m, 1H), 1.5 & 1.4 (m, 3H), 1.3 (t, 3H); ³¹P NMR (CD₃OD) δ 23.9, 25.37; MS: 719 (M+1).

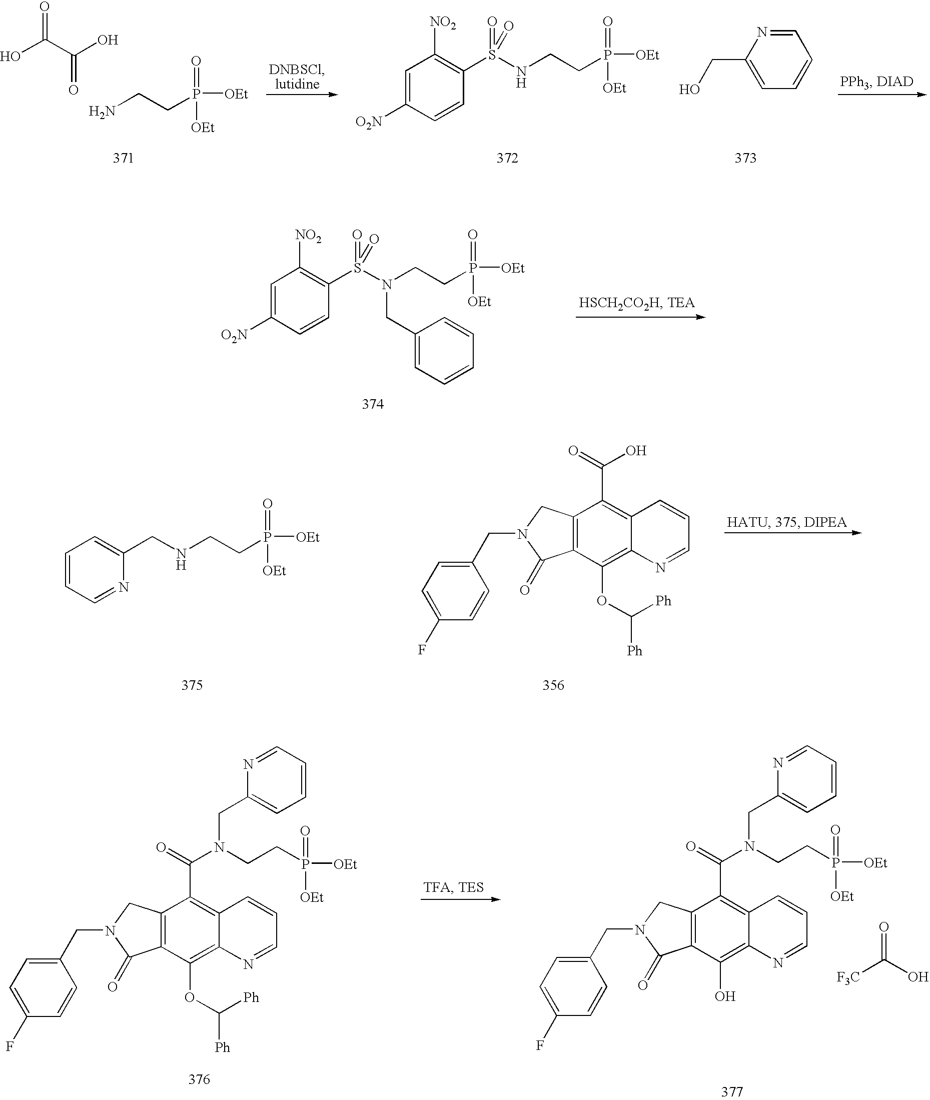

### Example 337

372: Commercially available Diethyl(aminoethyl)phosphonate oxalate 371 was partitioned between methylene chloride and saturated Na₂CO₃. The organic phase was dried (MgSO₄) then concentrated in vacuo to afford the freed amine (0.115 g, 0.635 mmol) which was dissolved in methylene chloride (1.3 mL). The solution was treated with 2,6-Lutidine (0.148 mL, 1.27 mmol) and 2,4-Dintrobenzene-sulfonyl chloride (0.254 g, 0.953 mmol) and stirred at room temperature for 1 hour. The reaction was quenched with acetic acid (0.3 mL), diluted with ethyl acetate then washed with saturated NH₄Cl and brine (twice), dried (NaSO₄), filtered and concentrated. The residue was purified by chromatography on silica gel (4/1—ethyl acetate/hexane) to afford the desired product 372 (0.200 g, 77%) as a solid.

374: To a solution of the protected amine 372 (0.99 g, 0.24 mmol) dissolved in methylene chloride (0.600 mL) was added triphenyl phosphine (0.190 g, 0.73 mmol) and 2-pyridyl carbinol 373 (0.079 g, 0.73 mmol). The reaction mixture was cooled to 0° C. under nitrogen atmosphere then Azodicarboxylic acid diisopropyl ester (DIAD) (0.155 mL, 0.73 mmol) was added. The reaction was stirred at room temperature for 2 hours then diluted with ethyl acetate, washed with saturated NH₄Cl (twice) and brine (twice), dried (NaSO₄), filtered and concentrated. The residue was purified by chromatography on silica gel (100%—ethyl acetate) to afford the desired product 374 (0.096 g, 60%) as a solid.

375: To a solution of compound 374 (0.095 g, 0.19 mmol) in methylene chloride (0473 mL) was added triethylamine (0.053 mL, 0.38 mmol) and mercaptoacetic acid (0.026 mL, 0.38 mmol). The reaction mixture was stirred under nitrogen atmosphere for 30 minutes then diluted with ethyl acetate, washed with saturated NaHCO₃ (twice), dried (NaSO₄), filtered and concentrated in vacuo without further purification to afford the product 375 (0.057 g, 100%).

376: This compound is synthesized in a similar fashion as compound 369 to afford the desired product 376 (69%).

377: This compound is synthesized in a similar fashion as compound 364 without need for purification by reversed phase HPLC to afford the desired product 377 (90%): ¹H NMR (CDCl₃) δ 8.95 (dd, 1H), 8.6-8.4 (m, 1H), 8.05 (dd, 1H), 7.6 (m, 1H), 7.4 (m, 1H), 7.3 (m, 2H), 7.05 (m, 3H), 6.75 (d, 1H), 5.1-4.3 (m, 6H), 4.15 (q, 4H), 3.6-3.4 (m, 2H), −2.5-1.6 (m, 2H), 1.35 (m, 6H); ³¹P NMR (CDCl₃) δ 28.68, 26.39; MS: 607 (M+1).

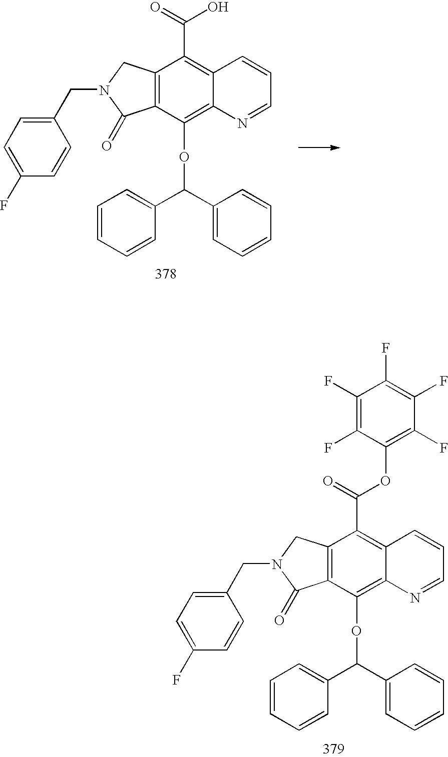

### Example 338

Carboxylic acid (0.2 g, 0.386 mmol) was dissolved in 2 mL of dimethylformamide. To this was added diisopropylamine (0.27 mL, 0.772 mmol), pentafluorophenol (0.142 g, 0.772 mmol), and O-(7-Azabenzotriazol-1-yl)-N,N,N′,N′-tetramethyluronium hexafluorophosphate (0.3 g, 0.772 mmol) and stirred at room temperature. After 1 hour, starting material consumed. Diluted crude with ethyl acetate, washed with saturated NH₄Cl solution, saturated NaHCO₃ and 2.5% LiCi solution, dried (Na₂SO₄), concentrated to give crude product (0.265 g, 100%.) ¹H NMR (CDCl₃) 9.24 (d, 1H), 9.10 (dd, 1H), 8.41 (s, 1H), 7.79 (d, 4H), 7.67 (dd, 1H), 7.43 (m, 8H), 7.21 (dd, 2H), 4.85 (s, 2H), 4.66 (s, 2H.) MS: 685 (M+1).

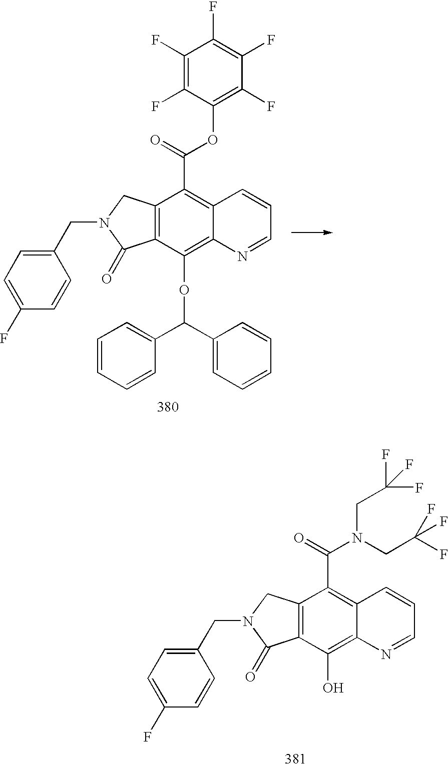

### Example 339

Pentafluorophenyl ester (0.063 mmol) in 0.5 mL of dimethylformamide. To this was added triethylamine (0.0056 mL, 0.0772 mmol), catalytic dimethylaminopyridine and bis-(2,2,2-trifluoroethyl)amine (0.023 g, 0.126 mmol) and then stirred at 70° C. After 15 hours, starting material was consumed and reaction quenched with 0.05 ml trifluoroacetic acid and 0.1 mL triethylsilance. Filtered crude through acrodisc filter and purified by reverse-phase HPLC with 1% TFA buffer. After lyophilization pure product was obtained (0.005 g, 0.0095 mmol, 15% for 2 steps.) ¹H NMR (CDCl₃) 9.63 (d, 1H), 9.0 (dd, 1H), 7.71 (dd, 1H), 7.38 (dd, 2H), 7.08 (dd, 2H), 4.80 (s, 2H), 4.76 (s, 2H), 4.65 (dd, 2H), 3.87 (dd, 2H.) MS: 516 (M+1).

Other compounds prepared in a similar fashion:

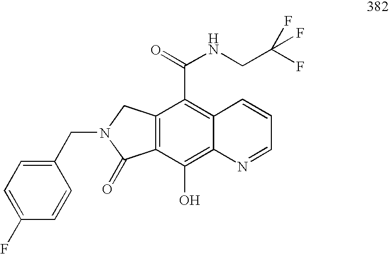

¹H NMR (CDCl₃) 9.55 (d, 1H), 9.06 (dd, 1H), 7.81 (dd, 1H), 7.38 (dd, 2H), 7.28 (dd, 2H), 4.79 (s, 2H), 4.77 (s, 2H), 4.44 (dd, 1H), 3.83 (dd, 1H.) MS: 434 (M+1). Yield: 26%

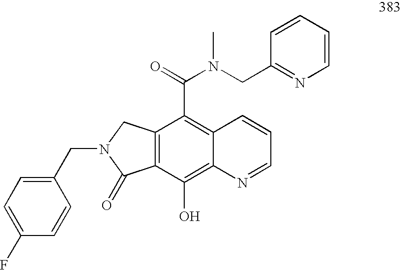

¹H NMR (CDCl₃) 9.05 (dd, 1H), 8.81 (dd, 1H), 8.25 (d, 1H), 8.13 (dd, 1H), 7.87 (d, 1H), 7.67 (m, 2H), 7.35 (m, 2H), 7.09 (dd, 2H) 5.18 (dd, 2H), 4.91 (d, J=14.1 Hz, 1H), 4.68 (d, J=14.7 Hz, 1H), 4.51 (d, J=17.7 Hz, 1H), 4.32 (d, J=17.4 Hz, 1H), 2.96 (s, 3H.) MS: 457 (M+1). Yield: 28%

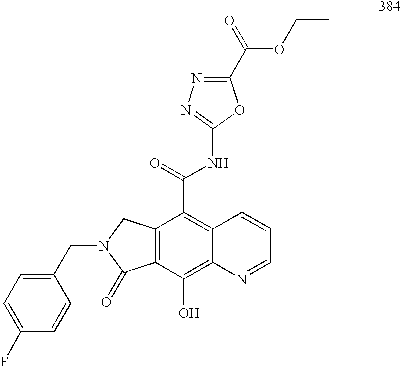

¹H NMR (CDCl₃) 9.44 (d, 1H), 9.02 (dd, 1H), 7.75 (dd, 1H), 7.35 (dd, 2H), 7.08 (dd, 2H), 4.80 (s, 2H), 4.78 (s, 2H), 4.56 (q, 2H), 1.48 (t, 3H.) MS: 492 (M+1). Yield: 32%

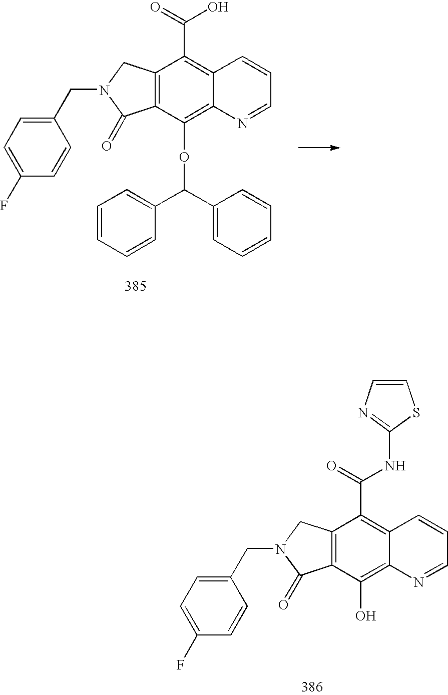

### Example 340

Carboxylic acid (0.02 g, 0.0386 mmol) was dissolved in 0.5 mL of dimethylformamide. To this was added 2-aminothiazole (0.0077 g, 0.0772 mmol), diisopropylethylamine (0.027 mL, 0.15 mmol), O-(7-Azabenzotriazol-1-yl)-N,N,N′,N′-tetramethyluronium hexafluorophosphate (0.03 g, 0.0772 mmol) and stirred at room temperature. After 15 hours, starting material was consumed. Filtered crude through acrodisc filter and purified by reverse-phase HPLC with 1% TFA buffer. After lyophilization pure product was obtained (0.01 g, 0.023 mmol, 60%.) ¹H NMR (CDCl₃) 9.02 (dd, 1H), 8.61 (d, 1H), 7.65 (dd, 1H), 7.55 (dd, 1H), 7.4 (m, 5H), 7.07 (dd, 2H), 4.78 (s, 2H), 4.66 (s, 2H.) MS: 435 (M+1).

Other compounds prepared in a similar fashion:

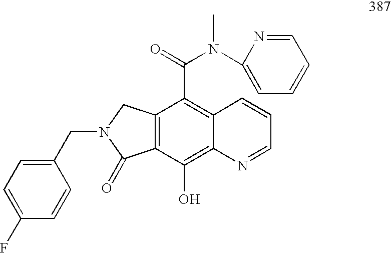

¹H NMR (CDCl₃) 9.02 (dd, 1H), 8.50 (d, 1H), 8.18 (dd, 1H), 7.65 (dd, 1H), 7.38 (m, 4H), 7.08 (dd, 2H), 7.00 (dd, 1H), 4.77 (dd, J=14.4 11.4 Hz, 2H), 4.49 (d, J=17.4 Hz, 1H), 4.19 (d, J=17.4, 1H), 3.61 (s, 3H.) MS: 443 (M+1). Yield: 8%

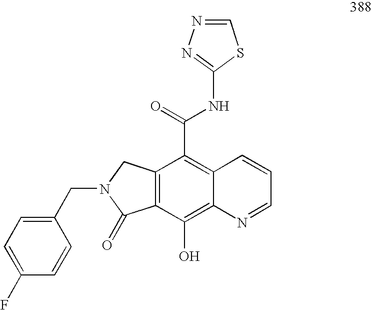

¹H NMR (CDCl₃) 9.02 (dd, 1H), 8.81 (s, 1H), 8.65 (d, 1H), 7.65 (dd, 1H), 7.4 (dd, 2H), 7.05 (dd, 2H), 4.74 (s, 2H), 4.64 (s, 2H.) MS: 436 (M+1). Yield: 40%

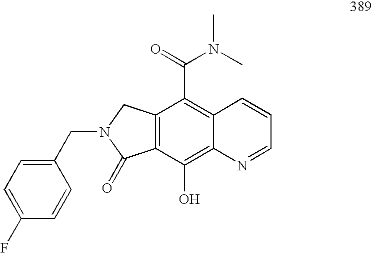

¹H NMR (CDCl₃) 9.07 (dd, 1H), 8.19 (d, 1H), 7.65 (dd, 1H), 7.4 (dd, 2H), 7.22 (dd, 2H), 4.83 (dd, 2H), 4.59 (d, J=8.7 Hz, 1H), 4.25 (d, J=8.7 Hz, 1H), 3.24 (s, 3H), 3.21 (s, 3H.) MS: 380 (M+1). Yield: 97%.

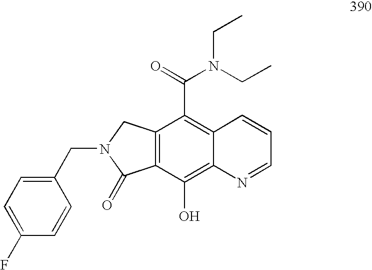

¹H NMR (CDCl₃) 9.20 (dd, 1H), 8.17 (d, 1H), 7.65 (dd, 1H), 7.4 (dd, 2H), 7.18 (dd, 2H), 4.89 (dd, 2H), 4.45 (d, J=17.1 Hz, 1H), 4.09 (d, J=16.8 Hz, 1H), 3.69 (q, 2H), 2.94 (q, 2H), 1.37 (t, 3H), 0.97 (t, 3H.) MS: 408 (M+1). Yield: 86%.

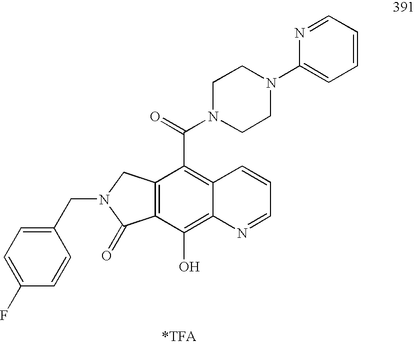

¹H NMR (CDCl₃) δ 9.02 (dd, 1H), 8.25 (dd, 1H), 8.17 (dd, 1H), 7.90 (dd, 1H), 7.64 (dd, 1H), 7.40 (dd, 2H), 7.07 (m, 4H), 4.86 (br m, 3H), 4.40 (br m, 3H), 3.90 (br s, 2H), 3.70 (br s, 2H), 3.46 (br s, 1H.) ¹⁹F NMR: −76.237 MS: 498 (M+1) Yield: 100%

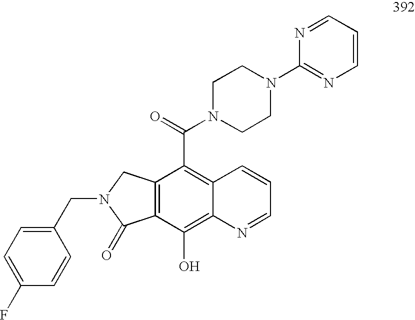

¹H NMR (CDCl₃) δ 8.99 (dd, 1H), 8.35 (d, 2H), 8.17 (d, 1H), 7.61 (dd, 1H), 7.40 (m, 3H), 7.07 (dd, 2H), 6.61 (dd, 1H), 4.86 (d, 1H), 4.67 (d, 1H), 4.53 (d, 1H), 4.25 (d, 1H), 4.03 (br s, 4H), 3.68 (br s, 2H), 3.27 (br s, 2H.) MS: 499 (M+1) Yield: 57%

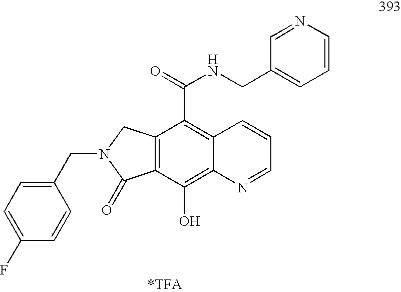

¹H NMR (CD₃SOCD₃) 9.14 (br s, 1H), 8.95 (dd, 1H), 8.15 (s, 1H), 8.06 (dd, 1H), 7.76 (dd, 4H), 7.55 (dd, 1H), 7.30 (m, 8H), 7.07 (dd, 2H), 4.95 (d, J=15 Hz, 1H), 4.70 (d, J=15 Hz, 1H), 4.42 (d, J=15 Hz, 1H), 4.14 (d, J=15 Hz, 1H), 3.94-3.79 (m, 4H), 3.41 (m, 2H), 2.99 (m, 2H.) MS: 588 (M+1). Yield: 51%

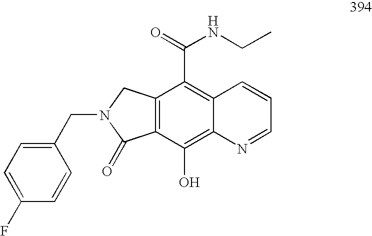

¹H NMR (CD₃SOCD₃) 8.95 (dd, 1H), 8.51 (s, 1H), 8.47 (d, 1H), 7.74 (dd, 1H), 7.39 (dd, 2H), 7.22 (dd, 2H), 4.70 (s, 2H), 4.48 (s, 2H), 3.33 (q, 2H), 1.15 (t, 3H.) MS: 380 (M+1) Yield: 100%

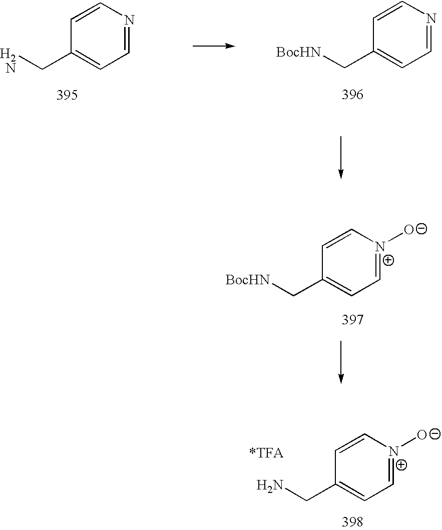

### Example 341

4-aminomethylpyridine was dissolved in tetrahydrofuran. To this was added catalytic DMAP and BOC anhydride. This was stirred at room temperature. After 15 hours, it was diluted with dichloromethane, washed with NH₄Cl, saturated NaHCO₃ solution, saturated brine, dried (NaSO₄), and concentrated to give crude product. The crude product was dissolved in 2:1 mixture dichloromethane and methanol. To this was added an excess of meta-chloroperbenzoic acid. After 20 hours, it was quenched with NaS₂CO₃, concentrated to oil, diluted with dichloromethane, washed with 1 N NaOH, and dried (Na₂SO₄), to give crude. It was then chromatographed to give pure product.

N-oxide dissolved in dichloromethane and trifluoroacetic acid. Stirred at room temperature. After one hour, azeotroped with toluene, diluted with dimethylformamide and purified by reverse-phase HPLC to give pure product (72% for 3 steps.) ¹H NMR (CD₃SOCD₃) 8.27 (d, 2H), 7.48 (d, 2H), 4.05 (s, 2H) ¹⁹F NMR: −74.171 MS: 125 (M+1)

Other pyridine N-oxide amino-methylamines prepared in a similar fashion.

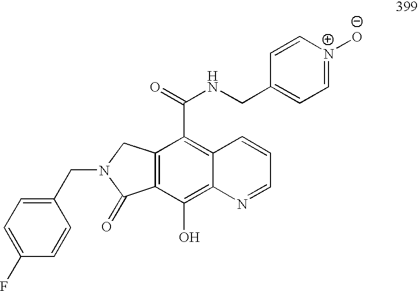

¹H NMR (CD₃SOCD₃) 9.11 (s, 1H), 8.59 (dd, 1H), 8.48 (d, 2H), 7.75 (dd, 1H), 7.39 (m, 4H), 7.22 (dd, 2H), 4.69 (s, 2H), 4.49 (s, 4H.) MS: 459 (M+1.) Yield: 44%

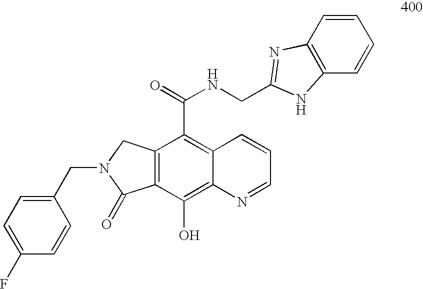

¹H NMR (CD₃SOCD₃) 9.28 (s, 1H), 8.97 (dd, 1H), 8.71 (d, 1H), 7.77 (dd, 1H), 7.73 (dd, 2H), 7.41 (dd, 2H), 7.36 (dd, 2H), 7.19 (dd, 2H), 4.88 (d, 2H), 4.69 (s, 2H), 4.58 (s, 2H.) MS: 482 (M+1.) Yield: 100%

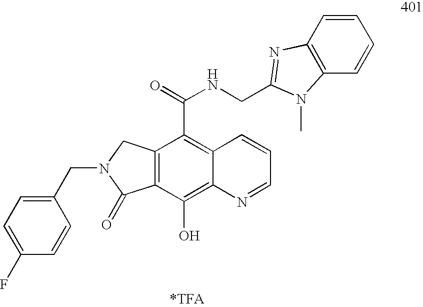

¹H NMR (CDCl₃) 8.87 (dd, 1H), 8.71 (d, 1H), 7.80 (dd, 1H), 7.53 (m, 4H), 7.27 (dd, 2H), 7.03 (dd, 2H), 5.12 (s, 2H), 4.73 (s, 2H), 4.61 (s, 2H), 4.14 (s, 3H.) ¹⁹F NMR: −76.215. MS: 496 (M+1.) Yield: 51%

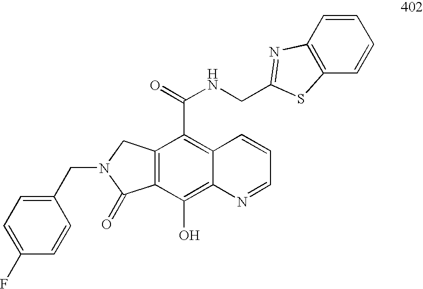

¹H NMR (CDCl₃) 8.91 (dd, 1H), 8.77 (d, 1H), 7.97 (dd, 2H), 7.55 (dd, 1H), 7.53 (m, 4H), 7.04 (dd, 2H), 5.18 (s, 2H), 4.68 (s, 2H), 4.58 (s, 2H.) MS: 499 (M+1.) Yield: 73%

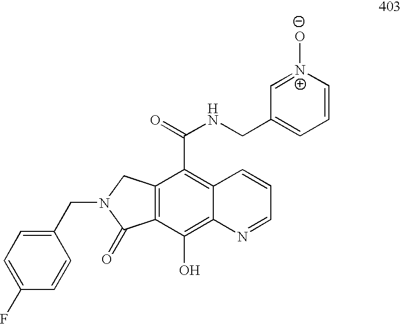

¹H NMR (CD₃SOCD₃) 9.06 (dd, 1H), 8.95 (dd, 1H), 8.47 (d, 1H), 8.23 (s, 1H), 8.17 (d, 1H), 7.75 (dd, 1H), 7.43 (m, 4H), 7.22 (dd, 2H), 4.68 (s, 2H), 4.48 (s, 4H.) MS: 459 (M+1.) Yield: 33%

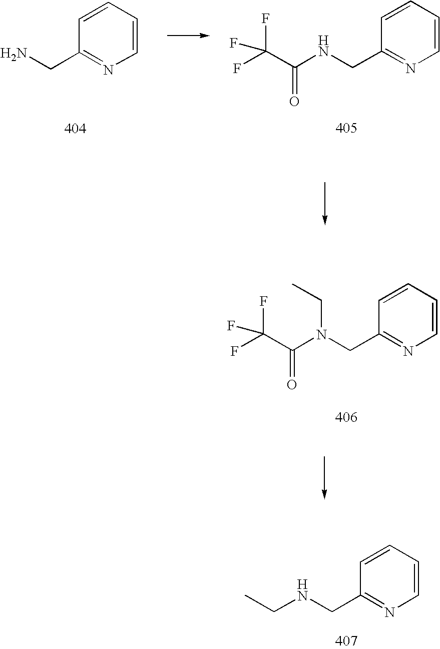

### Example 342

2-aminomethylpyridine was dissolved in dichloromethane and cooled to 0° C. To this was added diisopropylamine and trifluoroacetic anhydride. After two hours, diluted with dichloromethane, washed with saturated NaHCO₃, 1M HCl, dried (Na₂SO₄) and concentrated to give crude product.

Trifluoroacetamide was dissolved in dimethylformamide and cooled to 0° C. To this was added NaH and then iodoethane. After two hours, diluted with ethyl acetate, washed with saturated NH₄Cl, 2.5% LiCl solution, dried (Na₂SO₄) and concentrated to give crude product.

Crude product dissolved in tetrahydrofuran, methanol and H₂O. To this was added K₂CO₃, and then 1 N NaOH. Stirred at room temperature. After 15 hours, concentrated off organics, added saturated brine, extracted with ethyl acetate, dried (Na₂SO₄), concentrated to give crude product (30% for 3 steps.) ¹H NMR (CDCl₃) 8.64 (dd, 1H), 8.06 (dd, 1H), 7.75 (d, 1H), 7.65 (dd, 1H), 4.46 (s, 2H), 3.25 (q, 2H), 1.45 (t, 3H.) MS: 137 (M+1.)

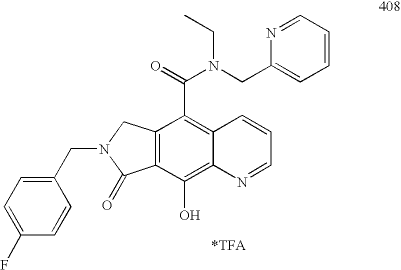

¹H NMR (CD₃SOCD₃) 8.59-6.99 (m, 11H), 4.87-4.12 (m, 4H), 3.4-3.1 (m, 2H), 1.19 (t, 3H.) ¹⁹F NMR: −74.620. MS: 471 (M+1). Yield: 29%.

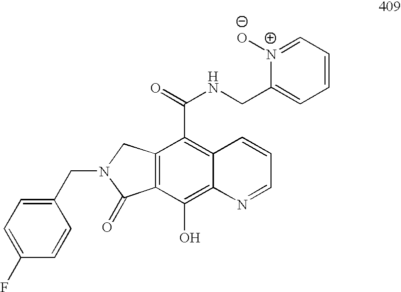

¹H NMR (CD₃SOCD₃) 9.05 (dd, 1H), 8.96 (dd, 1H), 8.55 (d, 1H), 8.34 (d, 1H), 7.75 (dd, 1H), 7.40 (m, 5H), 7.22 (dd, 2H), 4.72 (s, 2H), 4.61 (d, 2H), 4.55 (s, 2H.) MS: 459 (M+1.) Yield: 14%

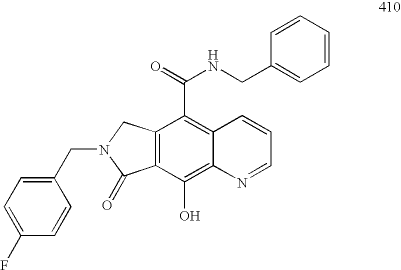

¹H NMR (CDCl₃) 8.87 (dd, 1H), 8.54 (d, 1H), 7.53 (dd, 1H), 7.40 (br s, 4H), 7.27 (br s, 2H), 7.07 (d, 2H), 6.66 (dd, 1H), 4.73 (d, 2H), 4.66 (s, 2H), 4.41 (s, 2H.) MS: 442 (M+1.) Yield: 64%

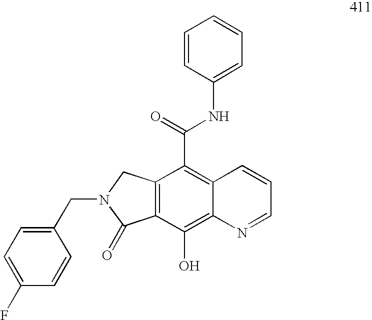

¹H NMR (CDCl₃) 9.25 (br s, 1H), 8.79 (dd, 1H), 8.56 (d, 1H), 7.94 (d, 2H), 7.50 (m, 3H), 7.20 (dd, 2H), 7.00 (dd, 2H), 4.35 (s, 2H), 4.27 (s, 2H.) MS: 428 (M+1.) Yield: 61%

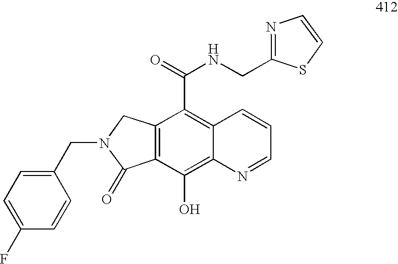

¹H NMR (CDCl₃) 8.83 (m, 2H), 8.08 (br s, 1H), 7.93 (s, 1H), 7.61 (dd, 1H), 7.46 (s, 1H), 7.27 (dd, 2H), 7.06 (dd, 2H), 5.14 (s, 2H), 4.67 (s, 2H), 4.53 (s, 2H.) MS: 449 (M+1.) Yield: 100%

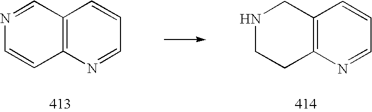

### Example 343

1,6-napthyridine was dissolved in methanol. To this was added catalytic 10% Pd/C and fitted with hydrogen atmosphere. After three hours, dilute with methanol, filtered through celite, concentrated to give pure product (59%.) ¹H NMR (CDCl₃) 7.96 (s, 2H), 6.34 (d, 1H), 4.56 (br s, 1H), 3.39 (m, 2H), 2.74 (dd, 2H), 1.97 (m, 2H.) MS: 135 (M+1.)

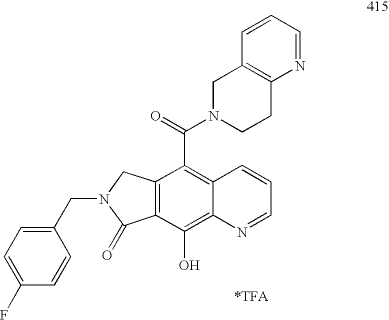

¹H NMR (CDCl₃) 9.04 (dd, 1H), 8.66 (s, 1H), 8.36 (d, 1H), 8.21 (dd, 2H), 7.66 (dd, 1H), 7.33 (dd, 2H), 7.08 (dd, 2H), 4.76 (s, 2H), 4.50 (d, 1H), 4.34 (d, 1H), 3.71 (m, 2H), 2.99 (dd, 2H), 2.04 (m, 2H.) MS: 469 (M+1.) Yield: 96%

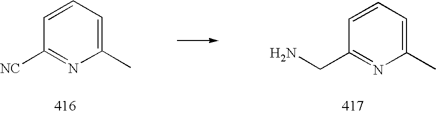

### Example 344

2-cyano-6-methylpyridine was dissolved in diethyl ether and cooled to 0° C. Slowly LiAlH₄ (1M in THF) was added. After five minutes, diluted with wet ether, quenched with 1M NaOH, filtered, concentrated to give crude product. ¹H NMR (CDCl₃) 7.55 (dd, 1H), 7.09 (d, 1H), 7.02 (d, 1H), 3.93 (s, 2H), 2.54 (s, 3H.) MS: 123 (M+1.)

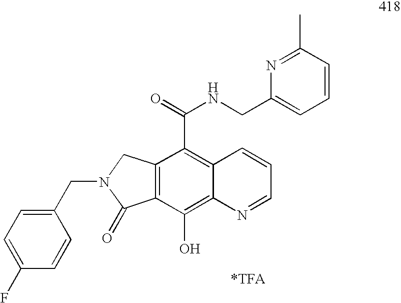

¹H NMR (CDCl₃) 9.24 (br s, 1H), 8.99 (dd, 1H), 8.71 (d, 1H), 8.28 (dd, 1H), 7.97 (d, 1H), 7.60 (dd, 2H), 7.35 (dd, 2H), 7.07 (dd, 2H), 4.97 (d, 2H), 4.78 (s, 2H), 4.65 (s, 2H), 2.84 (s, 3H.) ¹⁹F NMR: −76.318. MS: 457 (M+1.) Yield: 69%

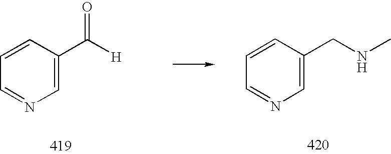

### Example 345

3-pyridine carboxyaldehyde was dissolved in ethanol. To this was added activated molecular sieves, methylamine and acetic acid. Stirred at room temperature for 1.5 hours. Then NaCNBH₃ was added. After another 1.5 hours, dilute with ethyl acetate, washed with saturated NaHCO₃ solution, dried (NaSO₄), concentrated to give crude. Chromatographed on silica to give pure product (14%.) ¹H NMR (CDCl₃) 8.57 (s, 1H), 8.52 (dd, 1H), 7.71 (d, 1H), 7.29 (dd, 1H), 3.79 (s, 2H), 2.48 (s, 3H.) MS: 123 (M+1.)

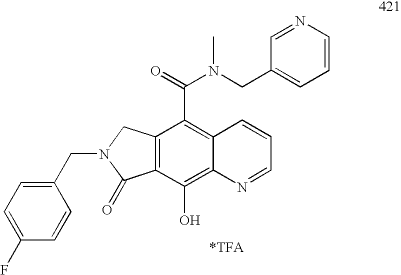

¹H NMR (CD₃SOCD₃) 8.96 (s, 1H), 8.74 (s, 1H), 8.67 (dd, 1H), 8.08 (dd, 2H), 7.72 (dd, 1H), 7.35 (m, 3H), 7.18 (dd, 2H), 4.88-4.29 (m, 4H), 2.69 (s, 3H.) ¹⁹F NMR: −74.890. MS: 457 (M+1.) Yield: 43%

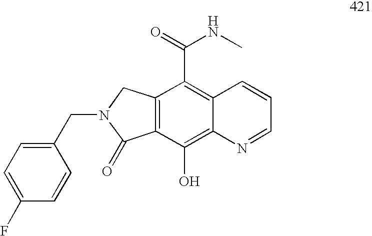

¹H NMR (CDCl₃) 8.93 (dd, 1H), 8.60 (d, 1H), 7.62 (dd, 1H), 7.32 (dd, 2H), 7.08 (dd, 2H), 6.40 (br s, 1H), 4.70 (s, 2H), 4.52 (s, 2H), 3.12 (s, 3H.) MS: 366 (M+1.) Yield: 39%

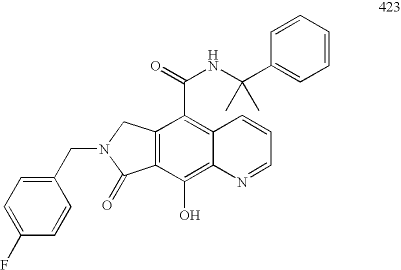

¹H NMR (CDCl₃) 8.91 (dd, 1H), 8.53 (d, 1H), 7.57 (dd, 1H), 7.50 (d, 2H), 7.36 (m, 5H), 7.07 (dd, 1H), 6.55 (s, 1H), 4.64 (s, 2H), 4.41 (s, 2H), 1.90 (s, 6H.) MS: 470 (M+1.) Yield: 17%

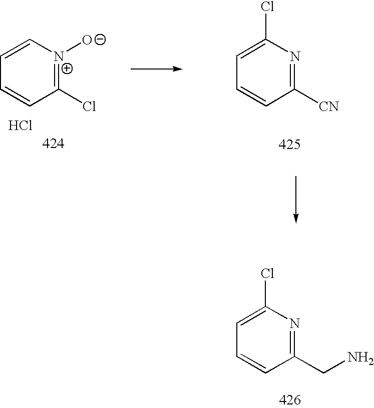

### Example 346

2-chloropyridine N-oxide HCl was dissolved in acetonitrile. To this was added triethylamine and trimethylsilyl cyanide and refluxed. After 15 hours, it was concentrated to oil, quenched with saturated Na₂CO₃ solution, extracted with dichloromethane, dried (Na₂SO₄), and concentrated to give crude. Chromatographed on silica to give pure product (37%.)

Cyanopyridine dissolved in diethyl ether and cooled to 0° C. To this was added, slowly, LiAlH₄ (1M in diethyl ether.) After 15 minutes, diluted with diethyl ether, washed with 1 M NaOH, dried (Na₂SO₄), concentrated to give crude. ¹H NMR (CDCl₃) 7.77 (dd, 1H), 7.42 (d, 1H), 7.25 (d, 1H), 3.96 (s, 2H.) MS: 143 (M+1.)

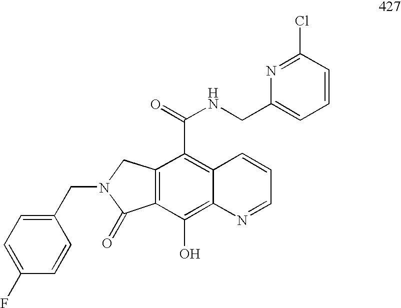

¹H NMR (CDCl₃) 8.91 (m, 2H), 7.73 (dd, 1H), 7.68 (dd, 1H), 7.32 (m, 4H), 7.05 (dd, 2H), 4.85 (d, 2H), 4.73 (s, 2H), 4.61 (s, 2H.) MS: 477 (M+1.) Yield: 65%.

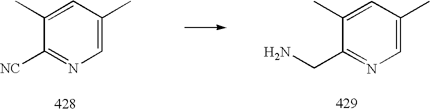

### Example 347

2-cyano-3.5-dimethylpyridine was dissolved in diethyl ether and cooled to 0° C. Slowly LiAlH₄ (1M in diethyl ether) was added. After five minutes, diluted with ether, quenched with 1M NaOH, filtered, concentrated to give crude product. ¹H NMR (CDCl₃) 8.23 (s, 1H), 7.25 (s, 1H), 3.92 (s, 2H), 2.28 (s, 3H), 2.26 (s, 3H.) MS: 137 (M+1.)

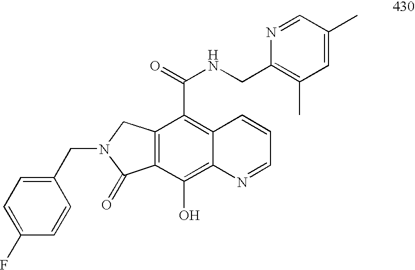

¹H NMR (CDCl₃) 9.20 (br s, 1H), 8.83 (dd, 1H), 8.72 (d, 1H), 8.43 (s, 1H), 7.84 (s, 1H), 7.55 (dd, 1H), 7.67 (dd, 2H), 7.05 (dd, 2H), 4.95 (d, 2H), 4.70 (s, 2H), 4.51 (s, 2H.) MS: 471 (M+1.) Yield: 7%

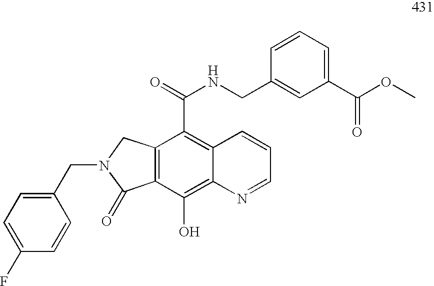

¹H NMR (CDCl₃) 8.83 (dd, 1H), 8.58 (d, 1H), 8.16 (s, 1H), 8.03 (d, 1H), 7.63 (dd, 1H), 7.55 (dd, 1H), 7.35 (dd, 2H), 7.05 (dd, 2H), 4.82 (d, 2H), 4.57 (s, 2H), 4.45 (s, 2H), 3.96 (s, 3H.) MS: 500 (M+1.) Yield: 97%

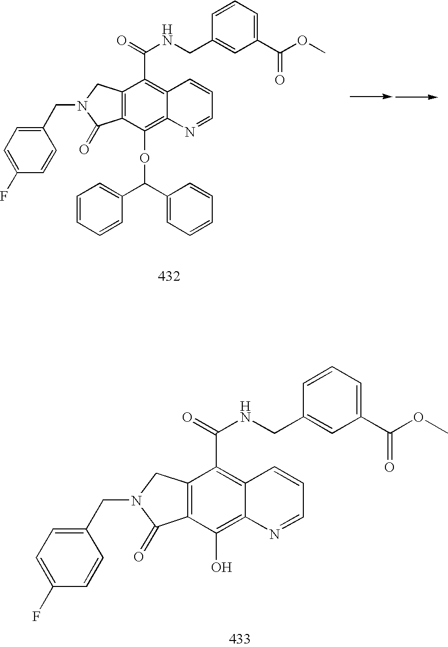

### Example 348

Methyl ester was dissolved in tetrahydrofuran and H₂O. To this was then added K₂CO₃ and then LiOH. After 15 hours, concentrated off solvent, diluted with dichloromethane, washed with 1 M HCl, driend (Na₂SO₄), concentrated to give crude. Chromatographed on silica to give pure product (31%.)

Carboxylic acid then dissolved in dichloromethane. To this was added triethylsilane and trifluoroacetic acid. Stirred at room temperature. After 10 minutes, concentrated off solvent, azeotroped with toluene, concentrated to give crude. Triturated with 1:1 ether/hexanes to give pure product. (94%.) ¹H NMR (CD₃SOCD₃) 9.15 (dd, 1H), 8.96 (dd, 1H), 8.47 (d, 1H), 7.98 (s, 1H), 7.87 (d, 1H), 7.71 (dd, 1H), 7.61 (d, 1H), 7.50 (dd, 1H), 7.37 (dd, 2H), 7.20 (dd, 2H.) 4.67 (s, 2H), 4.58 (d, 2H), 4.48 (s, 2H.) MS: 484 (M−1.)

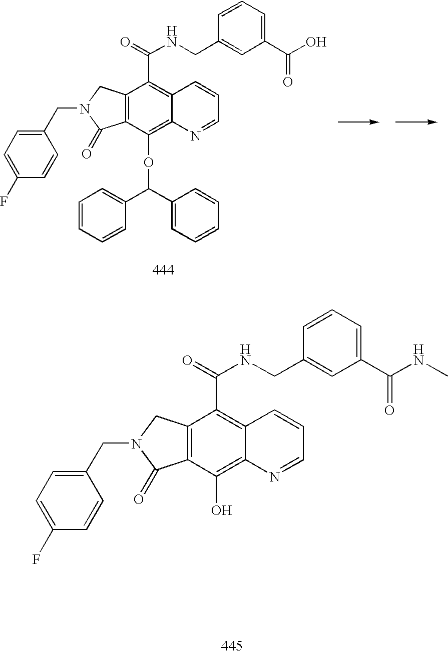

### Example 349

Carboxylic acid was dissolved in dimethylformamide. To this was added diisopropylethylamine, and O-(7-Azabenzotriazol-1-yl)-N,N,N′,N′-tetramethyluronium hexafluorophosphate and methylamine. Stirred at room temperature. After four hours, diluted with ethyl acetate, washed with saturated NH₄Cl, saturated NaHCO₃ solution, 2.5% LiCl solution, dried (Na₂SO₄), concentrated to give crude. Chromatographed on silica to give pure product (68%.).

Methyl amide was acid then dissolved in dichloromethane. To this was added triethylsilane and trifluoroacetic acid. Stirred at room temperature. After 10 minutes, concentrated off solvent, azeotroped with toluene, concentrated to give crude. Triturated with 1:1 ether/hexanes to give pure product. (74%.) ¹H NMR (CDCl₃) 8.74 (dd, 1H), 8.58 (d, 1H), 8.07 (s, 1H), 7.68 (d, 1H), 7.56 (m, 2H), 7.44 (dd, 1H), 7.23 (dd, 2H), 7.02 (dd, 2H), 4.82 (s, 2H), 4.45 (s, 2H), 4.41 (s, 2H), 2.96 (s, 3H.) MS: 499 (M+1.)

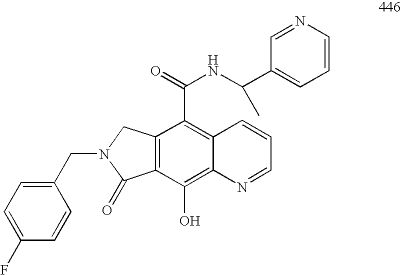

¹H NMR (CD₃SOCD₃) 9.13 (d, 1H), 8.96 (dd, 1H), 8.77 (s, 1H), 8.66 (d, 1H), 8.41 (d, 1H), 8.12 (d, 1H), 7.73 (dd, 1H), 7.67 (dd, 1H), 7.39 (dd, 2H), 7.23 (dd, 2H), 5.30 (ddd, 1H), 4.68 (s, 2H), 4.43 (s, 2H), 1.51 (d, 1H.) MS: 457 (M+1.) Yield: 53%.

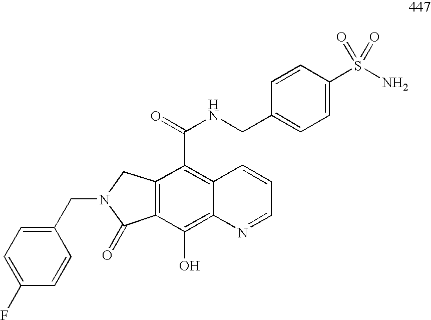

¹H NMR (CD₃SOCD₃) 9.11 (dd, 1H), 8.96 (dd, 1H), 8.48 (d, 1H), 7.83 (d, 2H), 7.73 (dd, 1H), 7.54 (d, 2H), 7.39 (dd, 2H), 7.22 (dd, 2H), 4.69 (s, 2H), 4.58 (d, 2H), 4.56 (s, 2H.) MS: 521 (M+1.) Yield: 41%.

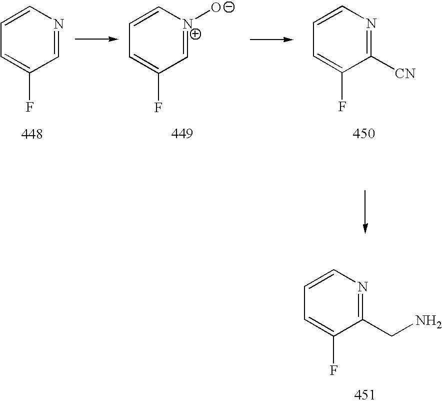

### Example 350

3-fluoropyridine was dissolved in a 2:1 mixture of dichloromethane and methanol. To this was added an excess of meta-chloroperbenzoic acid. Stirred at room temperature for 72 hours. Concentrated off solvent, diluted with dichloromethane, washed with 1M NaOH solution, dried (Na₂SO₄), concentrate to give crude.

Pyridine N-oxide was then dissolvd in acetonitrile. To this was added triethylamine and trimethylsilyl cyanide. Refluxed for 15 hours. Concentrated off solvent, diluted with dichloromethane, washed with saturated Na₂CO₃ solution, dried (Na₂SO₄), concentrated to give crude. Chromatographed on silica to give pure product (30%.)

Fluoro-cyanopyridine was dissolved in diethyl ether and cooled to 0° C. To this was added LiAlH₄ (1 M in ether.) After 5 minutes, diluted with diethyl ether, washed with 1 M NaOH, dried (Na₂SO₄), concentrated to give crude product. ¹H NMR (CDCl₃) 8.40 (dd, 1H), 7.38 (d, 1H), 7.28 (dd, 1H), 4.06 (s, 2H.) ¹⁹F NMR: −127.726. MS: 127 (M+1.)

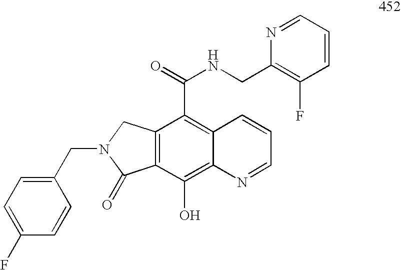

¹H NMR (CDCl₃) 8.93 (s, 1H), 8.77 (d, 1H), 8.32 (s, 1H), 7.59 (m, 2H), 7.49 (dd, 1H), 7.29 (m, 2H), 7.06 (dd, 2H), 4.96 (s, 2H), 4.70 (s, 2H), 4.57 (s, 2H.) MS: 461 (M+1.) Yield: 37%.

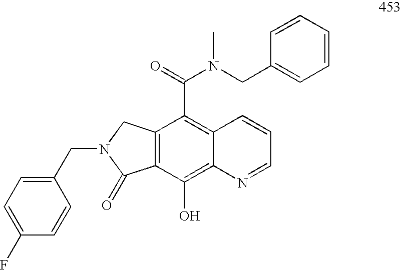

¹H NMR 90° C.(CD₃SOCD₃) δ 8.95 (dd, 1H), 8.08 (br s, 1H), 7.70 (dd, 1H), 7.37 (m, 7H), 7.17 (dd, 2H), 4.75 (br s, 4H), 4.17 (br s, 2H), 2.5 (s, 3H.) MS: 456 (M+1.) Yield: 63%

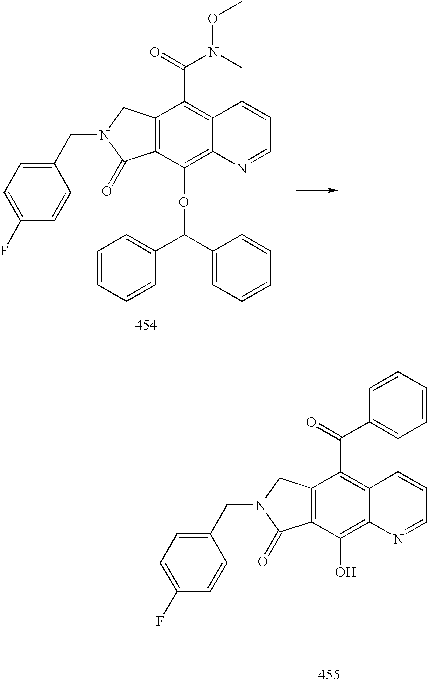

Weinreb amide was dissolved in tetrahydrofuran and cooled to 0 C. To this was addded an excess of phenyl magnesium bromide. After one hour, diluted with dichloromethane, washed with 1 M HCl, dried (Na2SO4), concentrated to give crude. Purified by reverse-phase HPLC with trifluoroacetic acid bufffer (2×) to give pure product. ¹H NMR (CDCl₃) 8.99 (s, 2H), 8.60 (d, 1H), 7.62 (m, 2H), 7.33 (m, 3H), 7.05 (m, 4H), 4.74 (s, 2H), 4.57 (s, 2H.) MS: 412 (M+1.) Yield: 2.3%

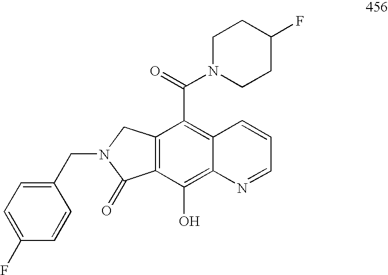

¹H NMR (CDCl₃) 8.99 (dd, 1H), 8.15 (dd, 1H), 7.61 (dd, 1H), 7.31 (dd, 2H), 7.08 (dd, 2H), 4.90-3.20 (m, 13H.) MS: 438 (M+1.) Yield: 26%

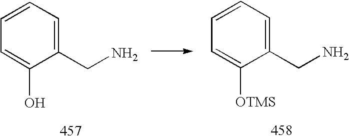

### Example 351

2-aminomethyl phenol was dissolved in acetonitrile. To this was added triethylamine and trimethylsilyl chloride. Stirred at 50° C. After 30 hours, diluted with dichloromethane, washed with NH₄Cl, dried (Na₂SO4), concentrate to give crude. Chromatographed on silica to give pure product (28%.) ¹H NMR (CDCl₃) 7.40 (d, 1H), 7.07 (dd, 1H), 6.98 (dd, 1H), 6.74 (d, 1H), 4.05 (s, 2H), 0.08 (s, 9H.) MS: 196 (M+1.)

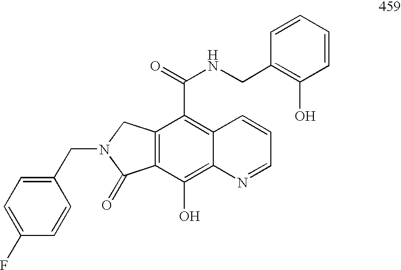

¹H NMR (CDCl₃) 8.75 (s, 1H), 8.51 (d, 1H), 7.51 (dd, 1H), 7.26 (m, 2H), 7.03 (m, 4H), 6.90 (dd, 2H), 4.68 (d, 2H), 4.59 (s, 2H), 4.42 (s, 2H.) MS: 458 (M+1.) Yield: 15%.

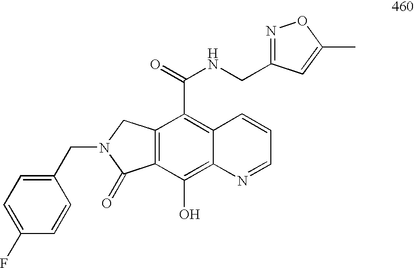

¹H NMR (CDCl₃) 8.85 (s, 1H), 8.78 (d, 1H), 7.57 (dd, 1H), 7.33 (dd, 2H), 7.08 (dd, 2H), 6.07 (s, 1H), 4.80 (d, 1H), 4.68 (s, 2H), 4.58 (s, 2H.) MS: 447 (M+1.) Yield: 65%.

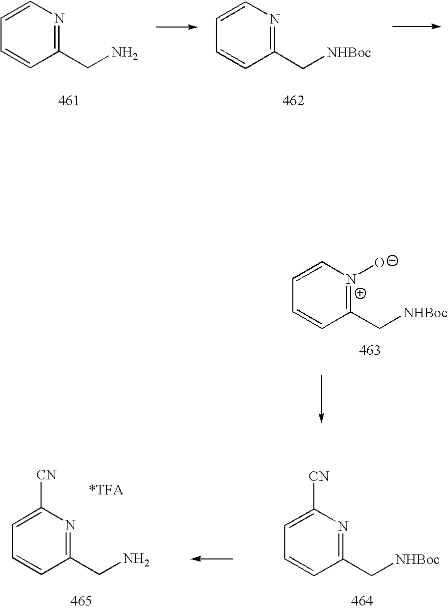

2-aminomethylpyridne was dissolved in tetrahydrofuran. To this was added catalytic dimethylaminopyridine and BOC₂₀. After 15 hours, diluted with dichloromethane, washed with saturated NH₄Cl, saturated NaHCO₃, dried (NaSO₄), concentrated to give crude product.

Protected amine was then dissolved in 2:1 dichloromethane and methanol. To this added meta-chloroperbenzoic acid. Stirred at room temperature. After four days, concentrate of solvent, diluted with dichloromethane, washed with 1 N NaOH, dried (Na₂SO₄), concentrated to give crude. Chromatographed on silica to give pure product.

Pyridine N-oxide was then dissolved in acetonitrile. To this was added triethylamine and trimethylsilyl cyanide. Refluxed. After 15 hours, dilute with dichloromethane, washed with saturated NaHCO₃ solution, dried (Na₂SO₄), concentrated to give crude product. Chromatographed on silica to give pure product.

2-cyanopyridine dissolved in a 2:1 mixture of dichloromethane and trifluoroacetic acid. Stirred at room temperature for one hour. Concentrated off solvent, azeotroped with toluene (2×), concentrate to give crude product. Triturate with hexanes to give pure product (20% for 4 steps.) ¹H NMR (CD₃SOCD₃) δ 8.14 (dd, 1H), 8.12 (dd, 1H), 7.82 (d, 1H), 4.31 (s, 2H.) MS: 134 (M+1.)

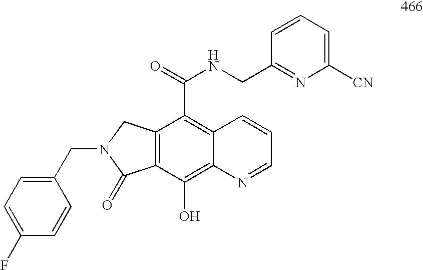

¹H NMR (CDCl₃) 8.98 (d, 1H), 8.90 (dd, 1H), 7.94 (dd, 1H), 7.70 (m, 3H), 7.34 (dd, 2H), 7.05 (dd, 2H), 4.98 (d, 2H), 4.69 (s, 2H), 4.61 (s, 2H.) MS: 468 (M+1.) Yield: 33%.

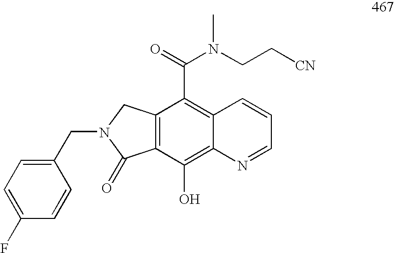

¹H NMR (CDCl₃) 9.06 (s, 1H), 8.33 (d, 1H), 7.71 (dd, 1H), 7.37 (dd, 2H), 7.08 (dd, 2H), 4.90 (d, 1H), 4.70 (d, 1H), 4.58 (d, 1H), 4.37 (d, 1H), 3.99 (m, 2H), 2.97 (br s, 2H.) MS: 419 (M+1.) Yield: 32%.

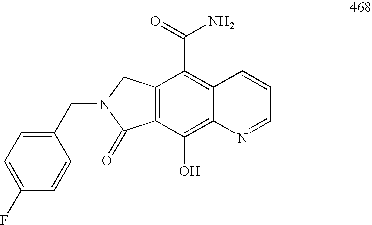

¹H NMR (CDCl₃ with 10 microliters of CF₃COOD) 9.49 (d, 1H), 9.25 (d, 1H), 8.23 (dd, 1H), 7.34 (dd, 2H), 7.13 (dd, 2H), 4.83 (s, 2H), 4.76 (s, 2H.) MS: 352 (M+1.) Yield: 35%

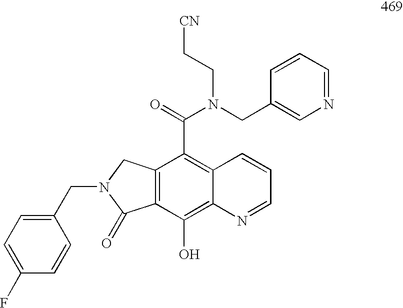

¹H NMR (CD₃SOCD₃) δ 8.95 (dd, 1H), 8.25 (d, 1H), 8.07 (s, 1H), 7.69 (m, 2H), 7.51 (dd, 1H), 7.32 (m, 2H), 7.21 (m, 3H), 4.94-3.4 (m, 6H), 3.03 (dd, 2H.) MS: 496 (M+1.) Yield: 34%.

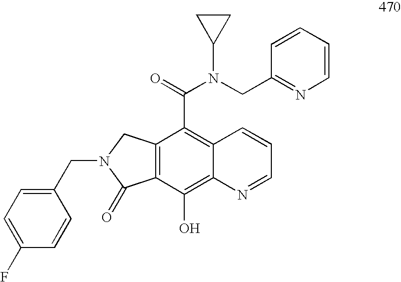

¹H NMR (CD₃SOCD₃) δ 8.96 (dd, 1H), 8.72 (d, 1H), 8.59 (d, 1H), 7.84 (dd, 1H), 7.78 (dd, 1H), 7.45 (d, 1H), 7.36 (dd, 2H), 7.20 (dd, 2H), 4.99-4.03 (m, 6H), 0.85 (br s, 1H), 0.40 (m, 1H), 0.33 (m, 1H), 0.21 (m, 1H), 0.067 (m, 1H.) MS: 483 (M+1.) Yield: 59%.

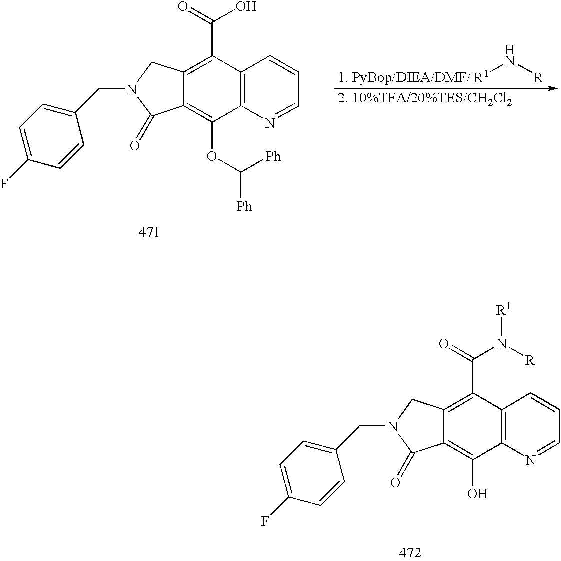

### Example 352

General Procedures for the Preparation of Compounds in Table A. Preparation of 9-Hydroxy-7-(4-fluoro-benzyl)-8-oxo-7,8-dihydro-6H-pyrrolo[3,4-g]quinoline-5-carboxamides by Array chemistry A stock solution of of 9-Benzhydryloxy-7-(4-fluoro-benzyl)-8-oxo-7,8-dihydro-6H-pyrrolo[3,4-g]quinoline-5-carboxylic acid was made from 680 mg of the acid dissolved in 17 ml of Dimethylformamide. To each of 34 vessels was added 500 ul of the acid stock solution. PyBop (819 mg=46.3 mg/r×n (1.2 eq) and diisopropylethylamine (457 ul=77.2 mmole/r×n (2.0 eq) were dissolved in DMF to give 8.5 ml total volume. To each vessel was added 250 ul of the PyBop/DIEA stock solution. Each of the 34 amines (amounts shown icharts below) were dissolved in DMF and added to the respect vial. The reactions were mixed by orbital shaking overnight at room temperature. A representative set of reactions were checked by LC/MS and shown to contain the desired product. The reaction mixtures were then concentrated in vacuuo overnight in a Genevac automated concentrator. After concentration, each reaction was treated with 10% trifluoroacetic acid/20% triethylsilane in methylene chloride for 30 minutes at room temperature. A representative set of reactions were checked by LC/MS and shown to contain the desired product. The reactions were concentrated in vacuuo in a Genevac automated concentrator for 3 hrs. The residues were dissolved in DMF and purified by reverse phase chromatography on a C-18 column using mass based collection. The fractions from each purification were analyzed by analytical HPLC/MS. Data for the analysis is shown below.

TABLE D

| Example | Structure | Compound | MW | Amount (mg) |
|---|---|---|---|---|
| 353 | 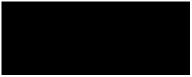 | 473 | 130.19 | 6.03 |
| 354 | 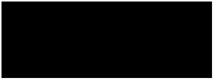 | 474 | 136.197 | 6.31 |
| 355 | 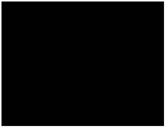 | 475 | 125.145 | 5.79 |
| 356 | 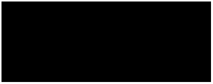 | 476 | 125.145 | 5.79 |
| 357 | 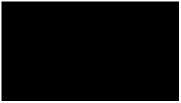 | 477 | 143.135 | 6.63 |
| 358 | 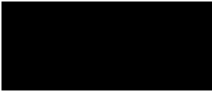 | 478 | 175.152 | 8.11 |
| 359 | 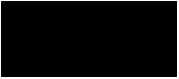 | 479 | 122.17 | 5.66 |
| 360 | 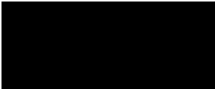 | 480 | 132.165 | 6.12 |
| 361 | 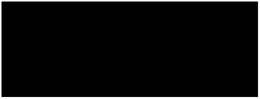 | 481 | 161.207 | 7.46 |
| 362 | 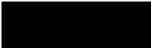 | 482 | 70.0944 | 3.25 |
| 363 | 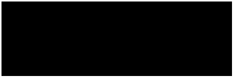 | 483 | 110.159 | 5.10 |
| 364 | 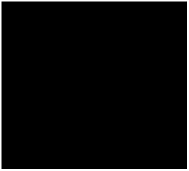 | 484 | 193.142 | 8.94 |
| 365 | 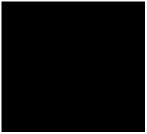 | 485 | 143.135 | 6.63 |
| 366 | 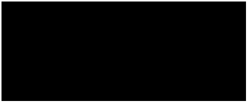 | 486 | 198.215 | 9.18 |
| 367 | 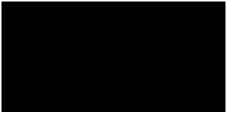 | 487 | 142.201 | 6.58 |
| 368 | 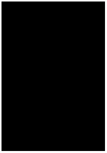 | 488 | 189.606 | 8.78 |
| 369 | 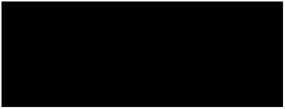 | 489 | 199.256 | 9.23 |
| 370 | 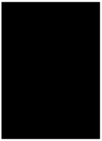 | 490 | 87.1211 | 4.03 |
| 371 | 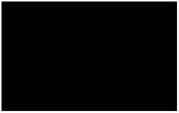 | 491 | 112.175 | 5.19 |
| 372 | 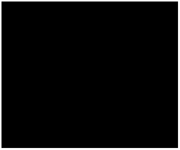 | 492 | 150.155 | 6.95 |
| 373 | 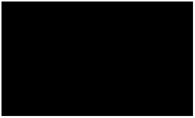 | 493 | 161.125 | 7.46 |
| 374 | 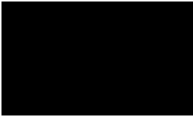 | 494 | 142.201 | 6.58 |
| 375 | 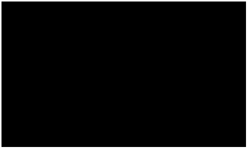 | 495 | 188.233 | 8.72 |
| 376 | 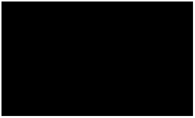 | 496 | 123.158 | 5.70 |
| 377 | 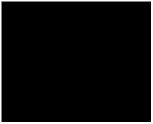 | 497 | 159.59 | 7.39 |
| 378 | 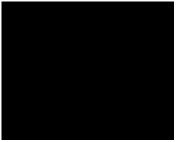 | 498 | 139.172 | 6.44 |
| 379 | 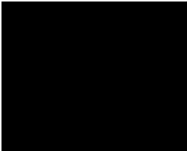 | 499 | 170.298 | 7.88 |
| 380 | 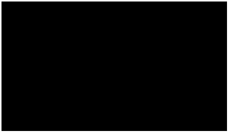 | 500 | 176.14 | 8.16 |
| 381 | 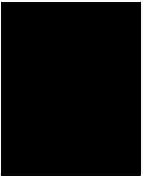 | 501 | 178.255 | 8.25 |
| 382 | 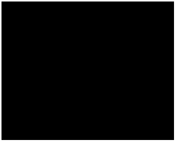 | 502 | 158.203 | 7.32 |
| 383 | 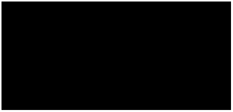 | 503 | 112.131 | 5.19 |
| 384 | 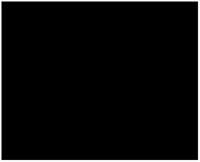 | 504 | 139.172 | 6.44 |
| 385 | 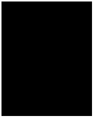 | 505 | 139.599 | 6.46 |
| 386 | 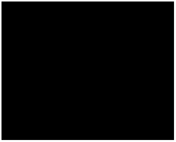 | 506 | 139.172 | 6.44 |

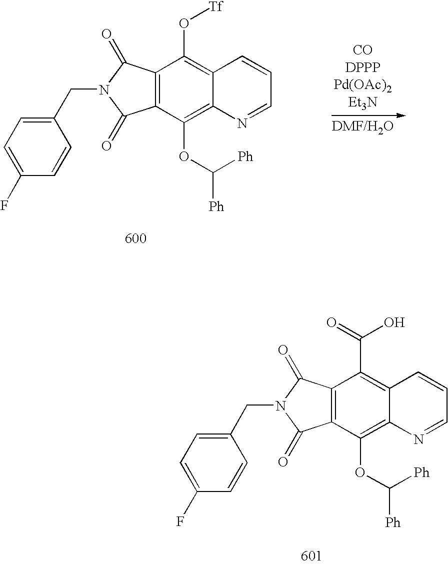

### Example 387

To a solution of trifluoro-methanesulfonic acid 9-benzhydryloxy-7-(4-fluoro-benzyl)-6,8-dioxo-7,8-dihydro-6H-pyrrolo[3,4-g]quinolin-5-yl ester 600 (1 g, 1.57 mmol) dissolved in DMF (10 mL) and water (0.5 mL) was added palladium (II) acetate (70 mg, 0.314 mmol, 0.2 eq)) and 1,3-bis(diphenylphosphine)-propane (192 mg, 0.471 mmol, 0.3 eq). The reaction mixture was stirred at room temperature and flashed with CO gas 3 to 6 times. It was then add triethylamine (480 μl, 3.45 mmol, 2.2 eq). The reaction mixture was heated at 70° C. for 3 hours under CO atmosphere. After cold to room temperature, the reaction was quenched by addition of 1N HCl (10 mL) and extracted with dichloromethane (2×30 mL). The organic phases were combined, dried over MgSO₄ and concentrated in vacuo. It gave 7 g of crude 9-benzhydryloxy-7-(4-fluoro-benzyl)-6,8-dioxo-7,8-dihydro-6H-pyrrolo[3,4-g]quinoline-5-carboxylic acid 601, which had concentration of 0.224 mmol per gram of crude mixture. MS (−): 531 (M−1).

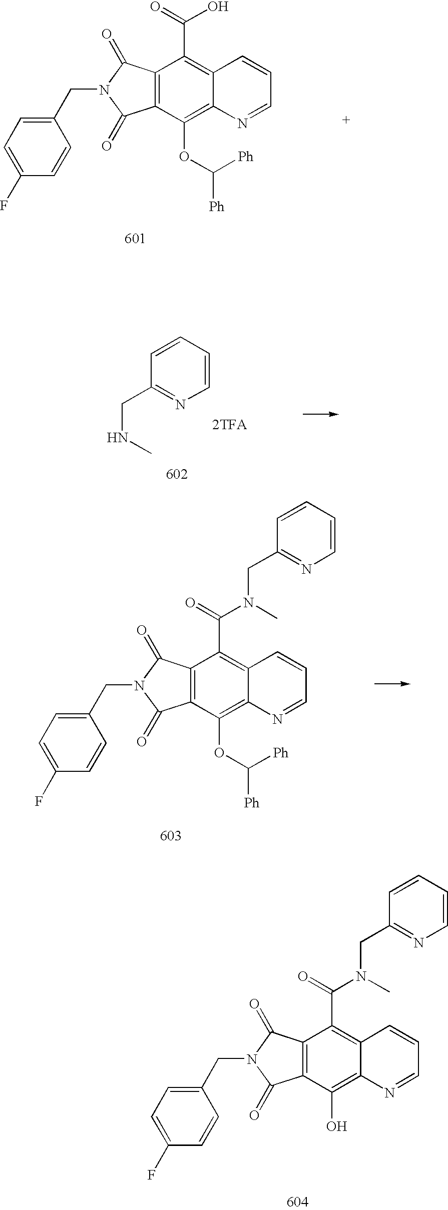

### Example 388

Step 1: To a solution of crude 9-benzhydryloxy-7-(4-fluoro-benzyl)-6,8-dioxo-7,8-dihydro-6H-pyrrolo[3,4-g]quinoline-5-carboxylic acid 601 (839 mg, 0.188 mmol) dissolved in DMF (4 mL) was added N,N-diisopropylethylamine (0.4 mL, 1.5 mmol), Q-(7-azabenzotriazol-1-yl)-N,N,N′,N′-tetramethyluronium hexafluorophosphate (HATU) (150 mg, 0.376 mmol). The reaction mixture was stirred at room temperature for 30 min. It was then added amine 602 (0.2 g, 0.564 mmol) and stirred overnight under nitrogen. The reaction was quenched by addition of 1N HCl (10 mL) and extracted with EtOAc (2×30 mL). The organic phases were combined, washed with brine, dried over MgSO₄ and concentrated in vacuo. The crude product was purified by HPLC to give the mixture of 603 and 604, 55 mg.

Step 2: This mixture was dissolved in dichloromethane (2 mL), and trifluoroacidic acid (200 μl) and triethylsilane (400 μl) were added. The reaction mixture was stirred at room temperature for ½ hours under an inert atmosphere then concentrated in vacuo. The residue was purified by HPLC to afford 7-(4-Fluoro-benzyl)-9-hydroxy-6,8-dioxo-7,8-dihydro-6H-pyrrolo[3,4-g]quinoline-5-carboxylic acid methyl-pyridin-2-ylmethyl-amide 604, TFA salt, (25.6 mg, 0.037 mmol, 20%) as a yellow solid: ¹H NMR (CDCl₃) δ 9.0 (d, 1H), 8.8 (d, 1H), 8.4 (d, 1H), 8.3 (d, 1H), 8.2 (t, 1H), 7.7 (m, 2H), 7.6 (m, 1H), 7.4 (dd, 2H), 7.0 (dd, 2H), 5.6 (d, 1H), 5.1 (d, 1H), 4.8 (s, s, 2H), 2.9 (s, 3H); MS: 471 (M+1). HPLC conditions: mobile phase A was 0.1% TFA in water, mobile phase b was 0.1% TFA in CH₃CN; gradient from 5% to 60% B in 20 min; flow rate was 20 mL/min; column was Phenomenex, luna 5μ, C18 (2), 150 mm×21.1 mm.

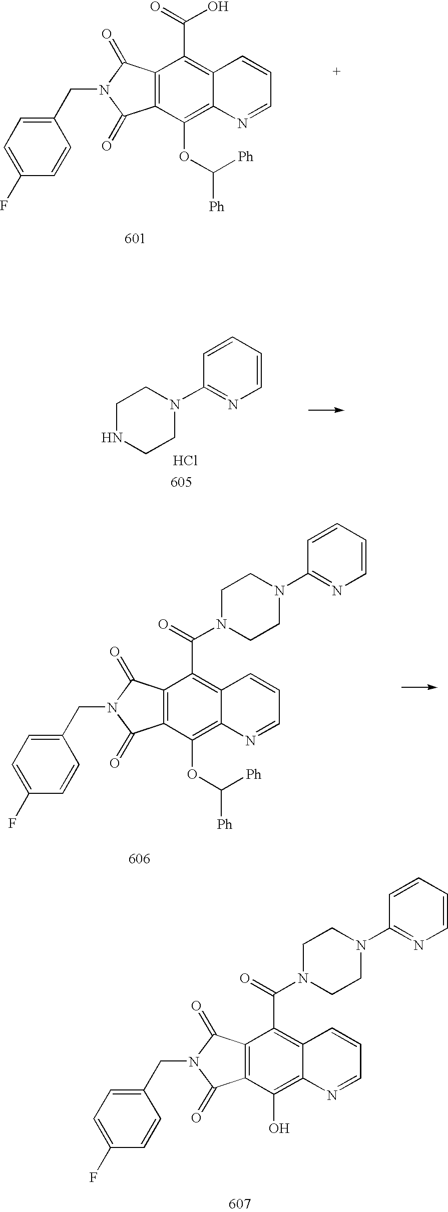

### Example 389

Compound 607 was made by the same manner as in Example 388. HPLC purification afforded 607 TFA salt, (39 mg, 0.076 mmol, 41%) as a yellow solid: ¹H NMR (CDCl₃) δ 9.0 (d, 1H), 8.3 (m, 2H), 7.8 (m, 2H), 7.7 (m, 1H), 7.4 (dd, 2H), 7.0 (m, 4H), 4.8 (s, s, 2H), 4.6-3.4 (m, 8H); MS: 512 (M+1).

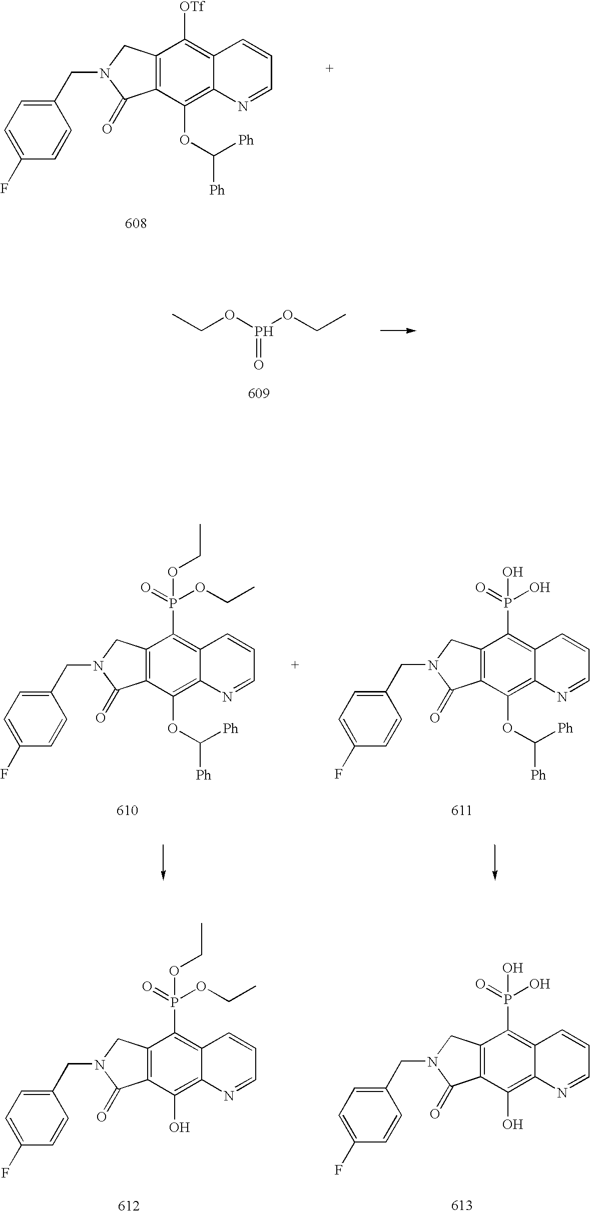

### Example 390

Step 1: To a solution of triflate 608 (50 mg, 0.08 mmol) dissolved in anhydrous CH₃CN (1 mL) was added N-methylmorpholine (11 μl, 0.104 mmol), diethylphosphite 609 (14 μl, 0.104). The mixture was flashed with argon three times. Tetrakis-(triphenylphosphine)-palladium (0) (3 mg, 0.024 mmol) was then added. The reaction mixture was heated to 75° C. under argon for 24 hours. No product was found by LC/MS. After cooling to room temperature, TEA (0.5 mL) and diethylphosphine (200 ul) were added. The mixture was degassed again and heated to 75° C. under argon for 24 hours. Cooling to room temperature, it was diluted with EtOAc (20 mL) and washed with 1N HCl, sat'd NaHCO₃ and brine. The organic phase was dried (MgSO₄). The crude product was purified by HPLC to afford intermediates 610 (2 mg) and 611 (4 mg).

Step 2: The experimental was carried out the same as in Example 388. After HPLC purification, it generated product 612, TFA salt, (0.4 mg), and product 613, TFA salt, (0.8 mg) separately.

¹H NMR (CDCl₃) for 612, δ 9.8 (d, 1H), 8.9 (d, 1H), 7.7 (m, 1H), 7.4 (m, 2H), 7.1 (t, 2H), 4.6 (s, 2H), 4.5 (s, 2H), 4.2 (m, 4H), 1.3 (m, 6H); MS: 445 (M+1).

¹H NMR (CDCl₃) for 613, δ 9.0 (d, 1H), 8.6 (d, 1H), 7.6 (m, 1H), 7.3 (m, 2H), 7.1 (t, 2H), 4.8 (s, 2H), 4.6 (s, 2H), 3.8 (s, 2H); MS: 411 (M+23).

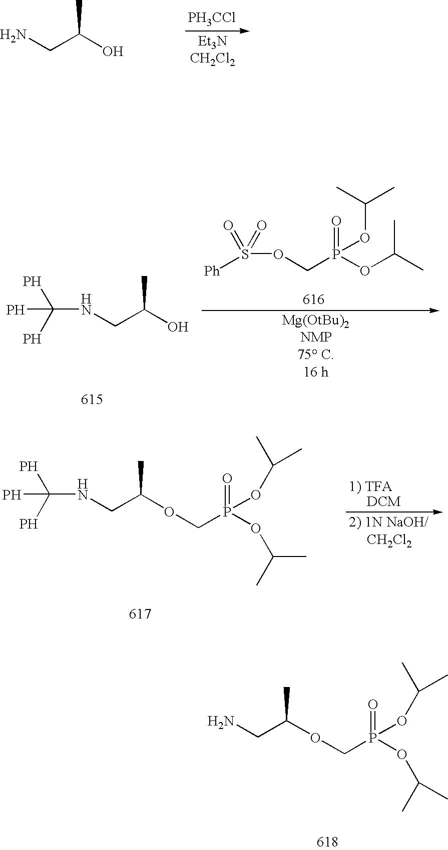

### Example 391

Step 1: To a solution of amino alcohol 614 (4 g, 0.053 mol) dissolved in dichloromethane (60 mL) was added TEA (15 mL) followed by trytl-Cl (14.84 g, 0.053 mol). The reaction mixture warmed up due to the reaction heat generating. The reaction was done in 2 h at room temperature. Filtered off the precipitation through a pile of Celite, the filtrate was concentrated and the residue was purified by flash chromatography on silica gel with EtOAc/Hexane (1/9 to 3/7). It yielded 16.8 g of compound 615, 99.4%.

Step 2: To a solution of 615 (16.8 g, 5.29 mmol) dissolved in NMP (80 mL) was added Mg(OtBu)₂ (18.1 g, 10.6 mmol), followed by 616 (21.4 g, 6.36 mmol). The mixture was heated to 75° C. for 16 h. After cooling to room temperature, water (about 150 mL) was added. The precipitate was collected by filtration (very slow). The sticky solid was dissolved in MeOH/CH₂Cl₂ (1/1) then concentrated. The crude mixture was purified by flash chromatography on silica gel with EtOAc/Hexane (1/9 to 1/1). It yielded 24 g of compound 617, 91%.

Step 3: Compound 617 (7.7 g, 15.5 mmol) was dissolved in 300 mL of 10% TFA/CH₂Cl₂ at room temperature. The reaction was done in 30 min. It was concentrated in vacuo and co-evaporated with CH₂Cl₂ two times which gave the TFA salt of 618. This salt was dissolved in 100 mL of CH₂Cl₂, then added 62 mL of 1N NaOH. The reaction mixture was stirred at room temperature for ½ hours. The layer was separated. The organic layer was concentrated to give the free amine 618.

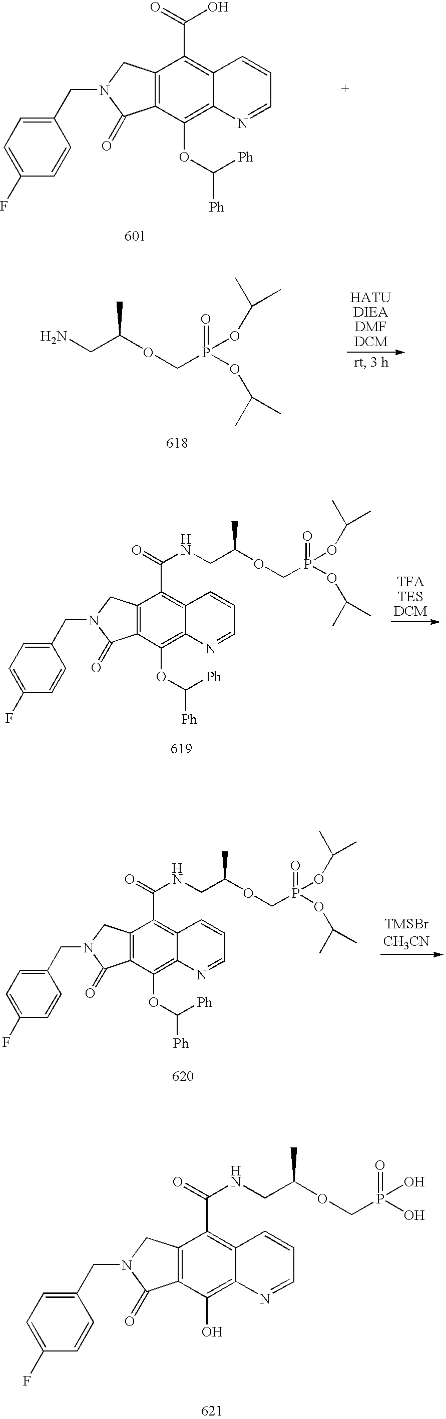

### Example 392

Compound 620 was made from coupling of acid 601 with amine 618 in the same manner as in Example 388. ¹H NMR (CD₃OD) δ 9.0 (d, 1H), 8.8 (d, 1H), 8.0 (s, 1H), 7.8 (m, 1H), 7.3 (m, 2H), 7.0 (t, 2H), 4.8 (dd, 2H), 4.7 (m, 2H), 4.6 (dd, 2H), 3.9 (m, 2H), 3.8 (m, 1H), 3.8 (m, 2H), 1.3-1.2 (m, 15H); ¹³P NMR: 20.7 ppm, s; MS: 588 (M+1).

### Example 393

Compound 620 (35 mg, 0.05 mmol) was dissolved in CH₃CN (1 mL) and cooled to 0° C. TMSBr (1 mL) was added slowly. The reaction was warmed to room temperature, and finished in 18 h. It was concentrated to give a crude residue, which was purified by HPLC (condition as in Example 388). Yield: 29 mg of compound 621, 94%. ¹H NMR (CD₃OD) δ 9.0 (d, 1H), 8.8 (d, 1H), 8.0 (s, 1H), 7.8 (m, 1H), 7.4 (m, 2H), 7.1 (t, 2H), 4.8 (s, 2H), 4.6 (s, 2H), 3.9-3.6 (m, 4H), 3.4 (m, 1H), 1.2 (s,s, 3H); ¹³P NMR: 19.9 ppm, s; MS: 504 (M+1).

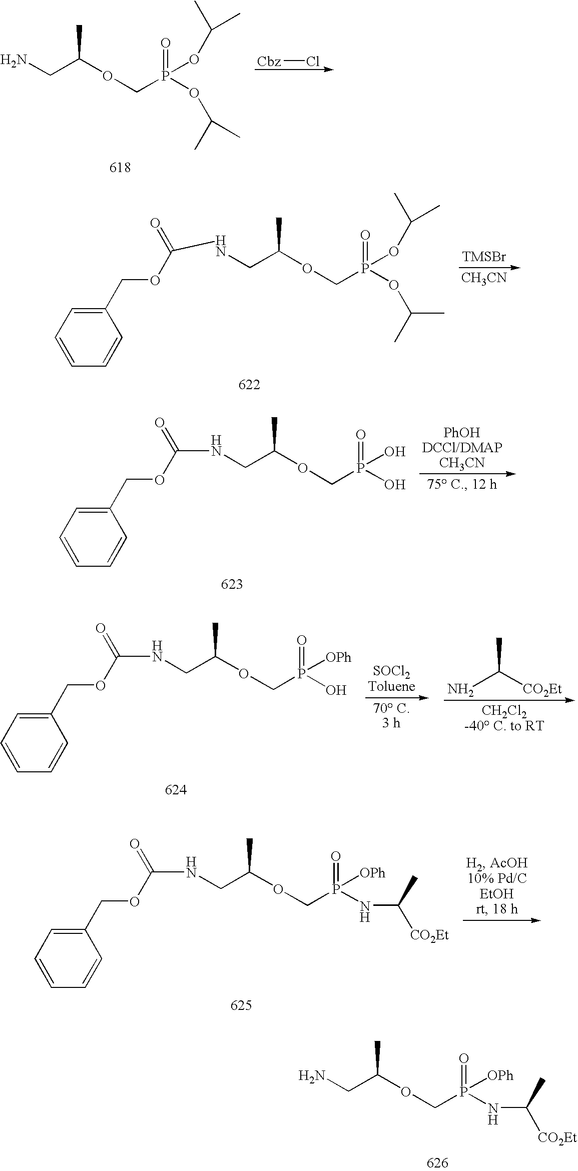

### Example 394

Step 1: To a solution of free amine 618 in the mixture of CH₂Cl₂ and 1N NaOH (from Example 391) was added Cbz-Cl (4.0 g, 23.25 mmol, 1.5 eq). The reaction was done in 18 h at room temperature. The layers were separated. The aqueous layer was extracted with CH₂Cl₂ twice. The organic layers were combined and dried (Na₂SO₄) and concentrated. The crude mixture was purified by flash chromatography on silica gel with EAOAc/Hexane (3/7) to afford pure 622, 2.6 g.

Step 2: To a solution of 622 (2.6 g, 6.7 mmol) dissolved in CH₃CN (30 mL) was added TMSBr (7 mL, 53.7 mmol) slowly at 0° C. The reaction mixture was stirred at 0° C. to room temperature over 3 hours under an inert atmosphere then concentrated in vacuo. The residue was dissolved in dichloromethane (100 mL) and 1N NaOH (150 mL). After stirring for 10 min, the layers were separated. The aqueous layer was added 1N HCl (175 mL), to pH=1. It was extracted with EtOAc three times. The organic layers were combined and dried (MgSO₄) and concentrated to give clean phosphonic acid 623, 2.1 g.

Step 3: To a solution of phosphonic acid 623 (1.8 g, 5.94 mmol) dissolved in CH₃CN (50 mL) was added PhOH (1.0 g, 10.7 mmol), DMAP (367 mg, 3 mmol) and DCC (1.5 g, 7.1 mmol). The reaction mixture was heated to 110° C. for 12 hours under an inert atmosphere then concentrated in vacuo. The residue was dissolved in EtOAc (50 mL)/1N NaOH (20 mL), and stirred for 10 min. The layers were separated. The aqueous layer which had sodium salt of 624 was acidified with 6N HCl slowly to pH=1. It was extracted with EtOAc three times. The organic layers were combined and dried (MgSO₄) and concentrated. The crude mixture was purified by flash chromatography on silica gel with MeOH/CH₂Cl₂ (10%, with 0.2% AcOH) to afford pure phosphonic acid monophenyl ester 624, 1.0 g, 44%.

Step 4: To a solution of phosphonic acid monophenyl ester 624 (1.0 g, 2.6 mmol) dissolved in toluene (20 mL) was added thionyl chloride (20 mL), DMF (4 drops). The reaction mixture was heated to 70° C. for 3 hours under an inert atmosphere then concentrated in vacuo. The residue was azeotropied with toluene twice to give the mono-chlorodate. This was redissolved in dichloromethane (20 mL) and cold to −40° C. The free base of alanine-ethylester dropwise. The mixture was kept at low temperature for 2 h, then room temperature overnight. After concentrated, it was purified by flash chromatography on silica gel with EtOAc/Hexane (1/1, with 0.2% TEA) to afford pure 625, 654 mg, 52%.

Step 5: To a solution of 624 (654 mg, 1.37 mmol) dissolved in EtOH (10 mL) was added AcOH (155 μl, 2.74 mmol) and 10% Pd/C (650 mg). The reaction mixture was stirred under an H₂ atmosphere for 18 h at room temperature. The solid was filtered off. The filtrate was concentrated in vacuo. The residue was dissolved in CH₂Cl₂ (50 mL)/sat'd Na₂CO₃ (50 mL), and stirred for 10 min. The layers were separated. The aqueous layer extracted with CH₂Cl₂ once more. The organic layers were combined and dried (Na₂SO₄) and concentrated. It afforded clean amine 625, 362 mg, 77%.

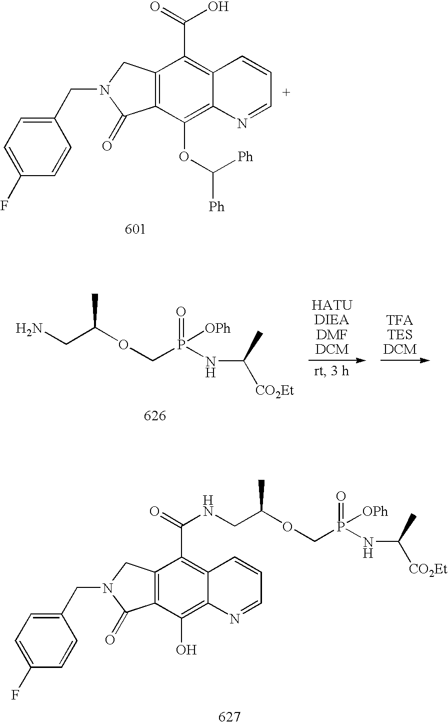

### Example 395

Compound 627 was synthesized by a method similar to Example 388. HPLC conditions: mobile phase A was water, mobile phase b was CH₃CN; gradient from 5% to 60% B in 20 min; flow rate was 20 mL/min; column was Phenomenex, luna 5μ, C18 (2), 150 mm×21.1 mm. ¹H NMR (CD₃OD) δ 8.8 (d, 1H), 8.6 (d, 1H), 7.6 (m, 1H), 7.4 (m, 2H), 7.3-7.0 (m, 7H), 5.6 (d, 1H), 4.8 (d, 2H), 4.6 (d, 2H), 4.1-3.4 (m, 8H), 1.3-1.1 (m, 9H); MS: 679 (M+1).

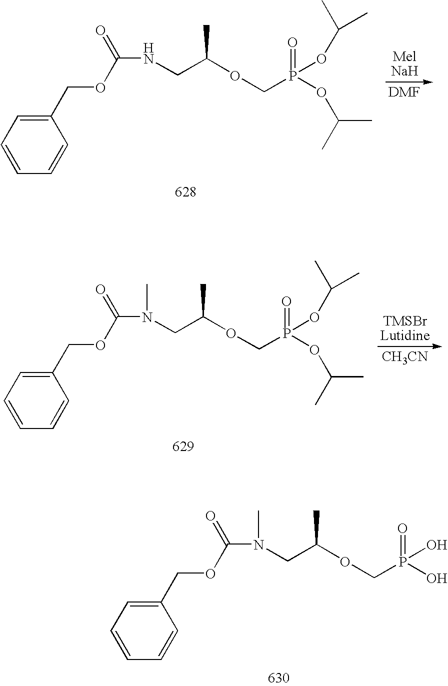

### Example 396

Step 1: To a solution of 628 (290 mg, 0.75 mmol) dissolved in DMF (1 mL) was added NaH (60%) (66 mg, 1.64 mmol) at 0° C. The reaction mixture was stirred for 20 min. MeI (102 μl, 1.64 mmol) was then added and kept at 0° C. under argon 1.5 hours. The mixture was diluted with EtOAc (20 mL) and washed with cold 1N HCl and brine. The organic phase was dried (MgSO₄), and concentrated in vacuo. It gave a clean compound 629, 330 mg, >100%.

Step 2: To a solution of 629 (0.75 mmol) dissolved in CH₃CN (30 mL) was added 2,6-lutidine (366 μl, 3.15 mmol) and cold to 0° C. TMSBr (3961, 3.0 mmol) was added slowly. The reaction mixture was stirred at 0° C. 2 h then room temperature 20 hours under an inert atmosphere. After completion of the reaction, it was cold to 0° C. again, then added 1N NaOH (10 mL) slowly. After stirring for 5 min, it was extracted with EtOAc. The aqueous layer was acidified with 1N HCl to pH=1. It was extracted with EtOAc/MeOH (9/1) three times. The organic layers were combined and dried (MgSO₄) and concentrated to give clean phosphonic acid 630, 214 mg, 89%

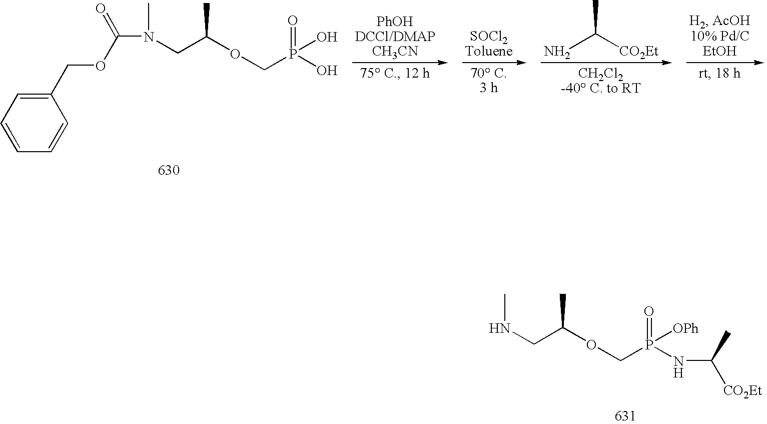

### Example 397

Conversion of compound 630 to 631 was done by the method as described in Example 394 (step 3, step 4, and step 5).

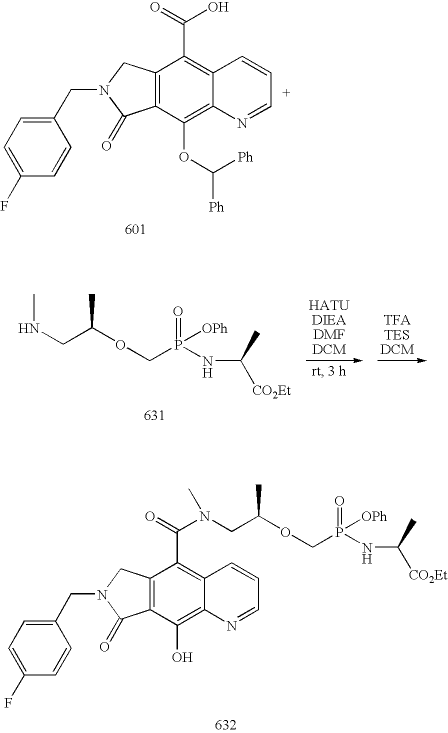

### Example 398

Compound 632 was synthesized by the method similar to Example 388. HPLC conditions: mobile phase A was water, mobile phase b was CH₃CN; gradient from 5% to 60% B in 20 min; flow rate was 20 mL/min; column was Phenomenex, luna 5μ, C18 (2), 150 mm×21.1 mm. ¹H NMR (CD₃OD) δ 8.8 (d, 1H), 8.6 (d, 1H), 7.6 (m, 1H), 7.4 (m, 2H), 7.3-7.0 (m, 7H), 5.6 (d, 1H), 4.8 (d, 2H), 4.6 (d, 2H), 4.1-3.4 (m, 8H), 2.8 (3H), 1.3-1.1 (m, 9H); MS: 693 (M+1).

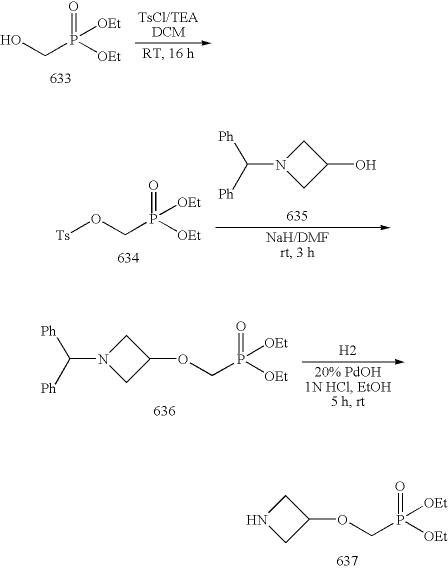

### Example 399

Step 1: To a solution of hydroxymethyl-phosphonic acid diethyl ester 633 (5 g, 29.7 mmol) dissolved in dichloromethane (20 mL) was added p-toluenesulfonyl chloride (5.66 g, 29.7 mmol), followed by slow addition of TEA (5.8 mL, 41.58 mmol) under nitrogen. The reaction mixture was stirred at room temperature for 16 hours. It was quenched with addition of water. The layers were separated. The organic layer was washed with 1N HCL, sat'd NaHCO₃ and dried (MgSO₄) and concentrated. The crude mixture was purified by flash chromatography on silica gel with Acetone/CH₂Cl₂ (5%) to afford pure toluene-4-sulfonic acid diethoxy-phosphorylmethyl ester 634, 7.5 g, 78%.

Step 2: To a solution of 1-benzhydryl-azetidin-3-ol 635 (1 g, 4.18 mmol) dissolved in anhydrous DMF (20 mL) was added sodium hydrate (552 mg, 13.8 mmol) and stirred for 30 min at room temperature under nitrogen. Tosylate 634 (2.02 g, 6.27 mmol) was then introduced by syringe. The reaction was done in 3 hours. It was quenched with cold 0.5 N HCl, extracted with EtOAc to give organic phase 1. The aqueous was treated with solid NaHCO₃ to pH 7-8, and extracted with EtOAc to give organic phase 2. The organic phases were combined and washed with brine, dried (MgSO₄) and concentrated. The crude mixture was purified by flash chromatography on silica gel with MeOH/CH₂Cl₂ (5%) to afford pure 636, 1.4 g, 85%.

Step 5: To a solution of 636 (941 mg, 2.4 mmol) dissolved in EtOH (20 mL) and 1N HCl (1 mL) was added 20% PdOH/C (1 g). The reaction mixture was stirred under an H₂ atmosphere for 6 h at room temperature. The solid was filtered off. The filtrate was concentrated in vacuo and lyophilized to afforded clean amine (HCl salt), 672 mg mg, 100%.

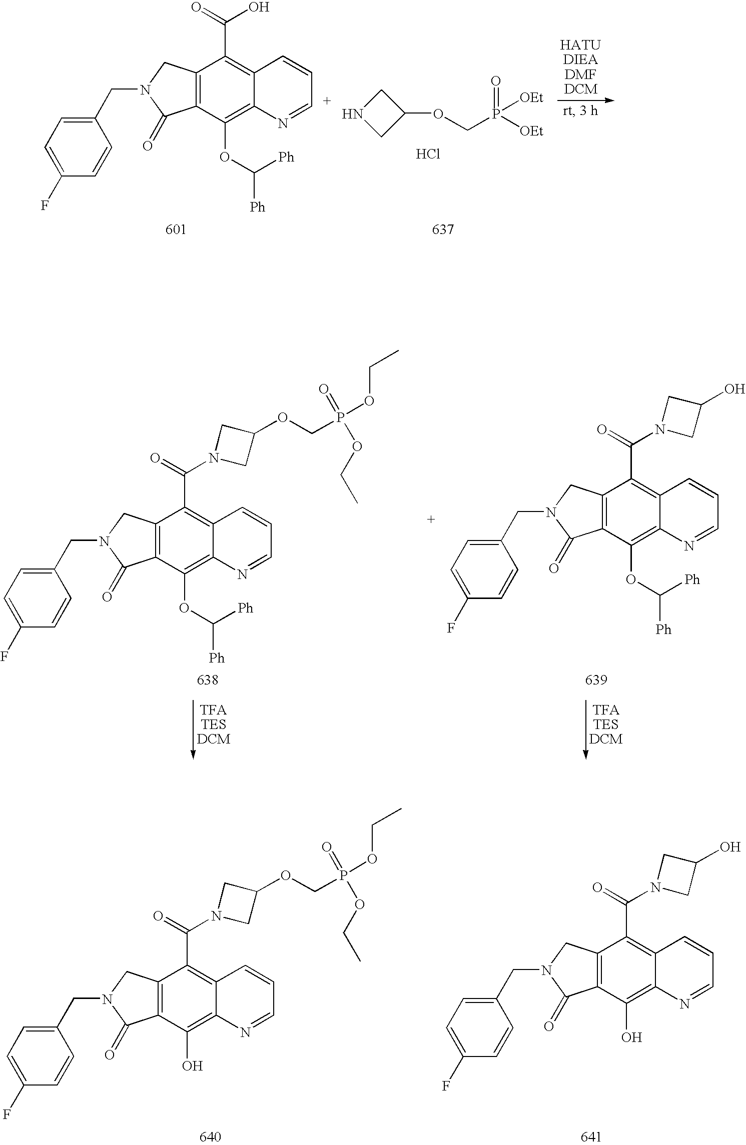

### Example 400

Compound 640 (13 mg) was synthesized by a method similar to Example 388.

Compound 641 (4 mg) was the by-product from this reaction.

The ratio of 638 to 639 was 24 mg to 12 mg from 42 mg of 601.

¹H NMR of compound 640 (CD₃OD) δ 9.0 (d, 1H), 8.5 (d, 1H), 7.8 (m, 1H), 7.4 (m, 2H), 7.1 (t, 2H), 4.8 (d, 2H), 4.5 (m, 2H), 4.4 (d, 2H), 4.1 (m, 4H), 4.0-3.6 (m, 4H), 3.3 (d, 2H), 1.3 (t, 6H); ³¹P NMR: 21.4 ppm; MS: 558 (M+1).

¹H NMR of compound 641 (CD₃OD) δ 9.0 (d, 1H), 8.5 (d, 1H), 7.8 (m, 1H), 7.4 (m, 2H), 7.1 (t, 2H), 4.8 (d, 2H), 4.7-4.4 (m, 4H), 4.0 (m, 2H), 3.6 (m, 1H); MS: 408 (M+1).

### Example 401

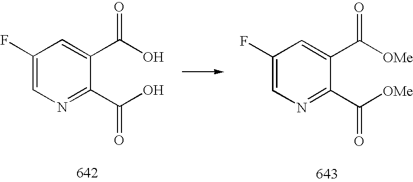

642 was prepared by methods similar to those described Roe and Hawkins (J. Am. Chem. Soc., 1949, 1785-1787 and Barfield and Water (Org. Magn. Res., 20, 2 1982, 92-101). A key oxidation to furnish 642 was described by Barre et al. (Synthesis, 2001, 16, 2495-2499) which was then esterified using standard conditions to furnish 643. ¹H NMR (300 MHz) CDCl₃ ∂: 3.98 (s, 3H), 3.85 (s, 3H) etc. ¹⁹F NMR (300 MHz) CDCl₃ ∂: −122.15. MS: (M+1) 214.1

### Example 402

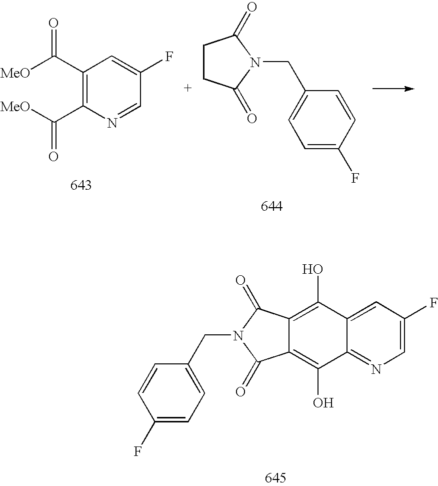

Following the procedure in Example 2, using NaHMDS as base at −78° C. compound 645 was prepared. ¹H NMR (300 MHz) CDCl₃ ∂: 4.72 (s, 2H) etc. ¹⁹F NMR (300 MHz) CDCl₃ ∂: −115.70, −115.71. MS (M+1) 357.1

### Example 403

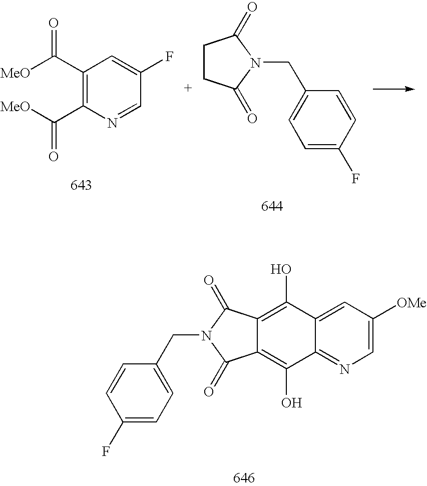

646 ¹H NMR (300 MHz) CDCl₃ ∂: 4.72 (s, 2H), 4.00 (s, 3H) etc. ¹⁹F NMR (300 MHz) CDCl₃ ∂: −115.77. MS (M+H).367.3

### Example 404

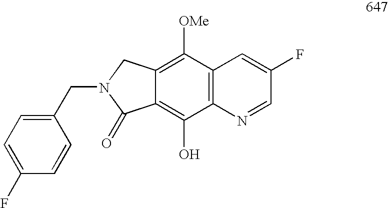

¹H NMR (300 MHz) CDCl₃ ∂: 8.84 (s, 1H), 8.09 (d, 1H), 7.33 (d, 2H), 7.09 (d, 2H), 4.78 (2H), 4.58 (s, 2H), 3.98 (s, 3H). ¹⁹F NMR (300 MHz) CDCl₃ ∂: −114.49, −125.02. MS: 379.23 (M+23)

### Example 405

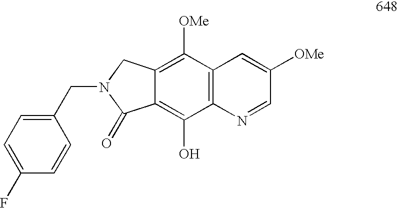

¹H NMR (300 MHz) CDCl₃ ∂: 8.72 (s, 1H), 7.68 (s, 1H), 7.36 (d, 2H), 7.04 (d, 2H), 4.77 (2H), 4.50 (s, 2H), 4.02 (s, 3H), 3.98 (s, 3H). ¹⁹F NMR (300 MHz) CDCl₃ ∂: −114.49. MS: 369.20 (M+1).

### Example 406

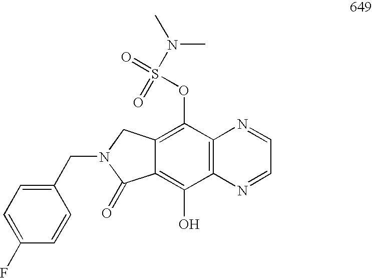

¹H NMR (300 MHz) CDCl₃ ∂: 8.98 (s, 1H), 8.95 (s, 1H), 7.34 (d, 2H), 7.04 (d, 2H), 4.78 (2H), 4.72 (s, 2H), 3.09 (s, 3H). ¹⁹F NMR (300 MHz) CDCl₃ ∂: −114.35. MS: 455.0 (M+1).

### Example 407

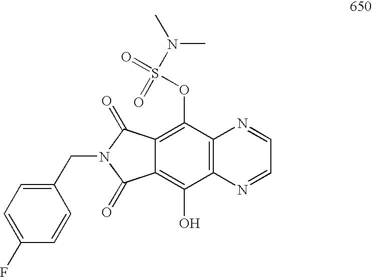

¹H NMR (300 MHz) CDCl₃ ∂: 9.16 (s, 1H), 8.96 (s, 1H), 7.50 (d, 2H), 7.04 (d, 2H), 4.87 (2H), 3.17 (s, 3H). ¹⁹F NMR (300 MHz) CDCl₃ ∂: −114.25. MS: 469.0 (M+23).

### Example 408

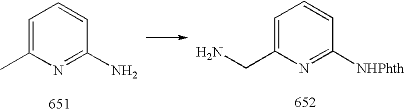

By standard procedures and those described in the literature, commercially available 651 was converted to 652 through several steps. ¹H NMR (300 MHz) CDCl₃ ∂: 6.58 (d, 1H), 6.37 (d, 1H). MS: 254.18 (M+1).

### Example 409

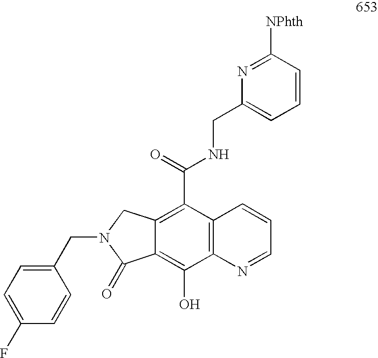

Amine 652 was coupled to a scaffold acid and the diphenylmethyl protecting group removed to furnish 653. ¹H NMR (300 MHz) CDCl₃ ∂: 9.39 (s, 1H), 9.05 (s, 1H), 8.71 (1, 1H), 8.52 (d, 1H), 7.92 (s, 1H), 7.75 (d, 2H), 7.50 (s, 1H), 7.34 (m, 3H), 7.04 (m, 3H), 5.13 (s, 2H), 5.02 (s, 2H), 4.84 (2H), 3.17 (s, 3H). ¹⁹F NMR (300 MHz) CDCl₃ ∂: −114.25, −76.11

### Example 410

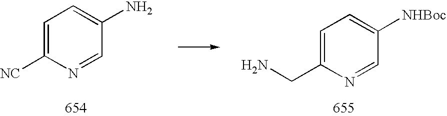

By standard procedures and those described in the literature, commercially available 654 was converted to 655 through several steps. ¹H NMR (300 MHz) CDCl₃ ∂: 8.48 (d, 1H), 8.12 (d, 1H), 7.85 (d, 1H), 6.77 (bs, 1H), 4.54 (AB, 2H), 1.48 (s, 9H). MS: 222.94 (M+H).

### Example 411

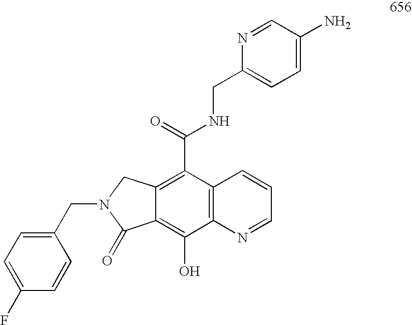

Amine 655 was coupled to the scaffold acid and the diphenylmethyl ether and Boc removed concomitantly to furnish 656. ¹H NMR (300 MHz) CD₃OD ∂: 8.92 (s, 1H), 8.59 (b s, 1H), 7.94 (bs, 1H), 7.69 (m, 3H), 7.39 (m, 2H), 7.23 (m, 1H), 7.11 (m, 3H), 4.76 (m, 4H), 4.52 (s, 2H). ¹⁹F NMR (300 MHz) CD₃OD ∂: −77.83.

### Example 412

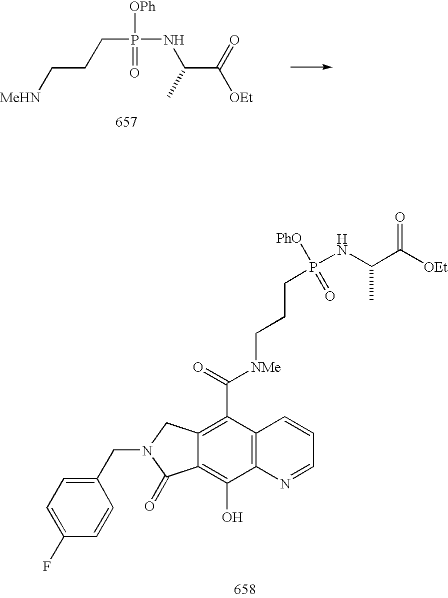

Amine 657 was obtained from the procedures similar to those described herein. Following its formation, it was then coupled to a carboxylic acid scaffold before the diphenylmethyl ether was removed to obtain 658, which was isolated as a mixture of diastereomers and rotamers. ¹H NMR (300 MHz) CD₃OD ∂: 8.95 (s, 1H), 8.11 (m, 1H), 7.62 (s, 1H), 7.27 (m, 7H), 7.06 (m, 2H), 7.94 (bs, 1H), 2.10 (m, 4H), etc. ¹⁹F NMR (300 MHz) CD₃OD ∂: −77.16, −114.71. ³¹P NMR (300 MHz) CD₃OD ∂:32.24, 32, 13, 32.03, 31.94, 30.96, 30.82. MS: 663.44 (M+H).

### Example 413

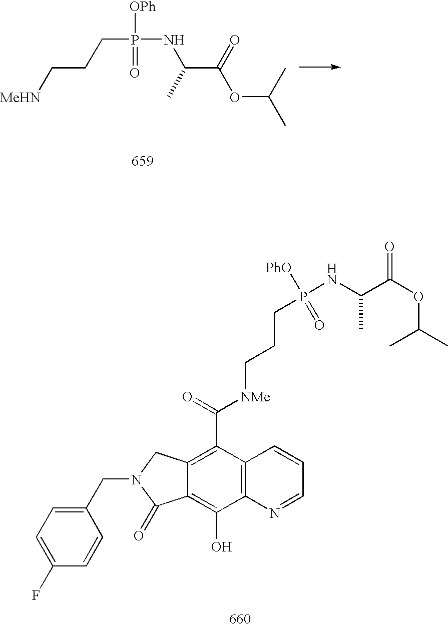

Amine 659 was obtained from the procedures similar to those described herein. Following its formation, it was then coupled to a carboxylic acid scaffold before the diphenylmethyl ether was removed to obtain 660, which was isolated as a mixture of diastereomers and rotamers. ¹H NMR (300 MHz) CD₃OD ∂: 8.97 (s, 1H), 8.10 (m, 1H), 7.57 (s, 1H), 7.27 (m, 7H), 7.06 (m, 3H), 3.21 (s, 3H, rotamer N-Me), 2.78 (s, 3H, rotamer N-Me). ¹⁹F NMR (300 MHz) CD₃OD ∂: −76.42, −114.62. ³¹P NMR (300 MHz) CD₃OD ∂:31.35, 31.23, 31.02, 30.93, 30.03, 29.92. MS: 677.25 (M+H).

### Example 414

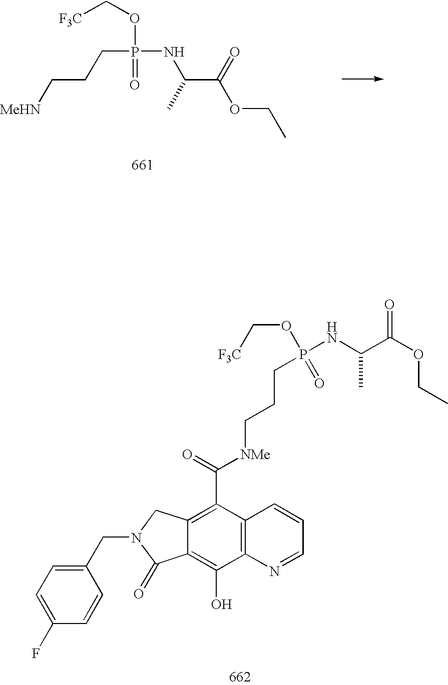

Amine 661 was obtained from the procedures similar to those described herein. Following its formation, it was then coupled to a carboxylic acid scaffold before the phenol was exposed to obtain 662, which was isolated as a mixture of diastereomers and rotamers.

¹H NMR (300 MHz) CDCl₃ ∂: 8.97 (s, 1H), 8.10 (m, 1H), 7.60 (s, 1H), 7.27 (m, 2H), 7.06 (m, 2H), 3.19 (s, 3H, rotamer N-Me), 2.78 (s, 3H, rotamer N-Me).

¹⁹F NMR (300 MHz) CDCl₃ ∂: −77.43, −114.66

³¹P NMR (300 MHz) CDCl₃ ∂:35.91, 35.85, 35.11, 34.73, 34.18

MS: 669.27 (M+H).

### Example 415

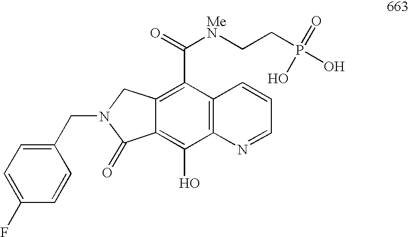

¹H NMR (300 MHz) CD₃OD ∂: 8.99 (d, 1H), 8.46 (d, 1H), 7.82 (d, 1H), 7.41 (s, 2H), 7.05 (m, 2H), 4.67 (d, 1H), 4.88 (d, 1H), 3.88 (m, 1H), 3.20 (s, N-Me rotamer, 3H), 2.86 (s, N-Me rotamer, 3H), 2.24 (m, 1H), 1.91 (m, 1H).

³¹P NMR (300 MHz) CDCl₃ ∂: 25.62, 23.88

MS: 474.27 (M+H).

### Example 416

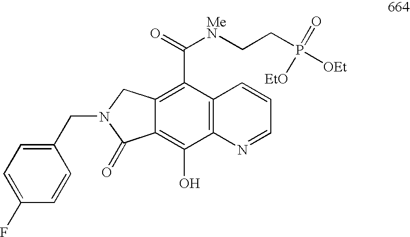

¹H NMR (300 MHz) CD₃OD ∂: 9.02 (d, 1H), 8.23 (d, 1H), 7.62 (d, 1H), 7.32 (s, 2H), 7.08 (m, 2H), 4.88 (d, 2H), 4.70 (d, 2H), 4.56 (d, 2H), 4.19 (m, 4H), 3.92 (m, 2H), 3.19 (s, N-Me rotamer, 3H), 2.83 (s, N-Me rotamer, 3H), 2.32 (m, 2H), 1.91 (m, 1H), 1.38 (m, 6H).

¹⁹F NMR (300 MHz) CDCl₃ ∂: −76.44, −114.56

³¹P NMR (300 MHz) CDCl₃ ∂: 28.372, 26.24

MS: 530.28 (M+H).

### Example 417

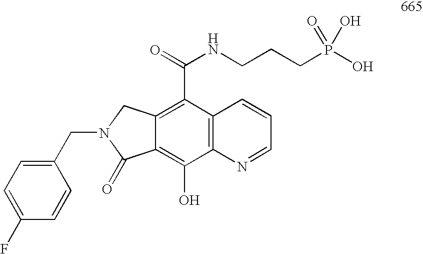

¹H NMR (300 MHz) CD₃OD ∂: 9.01 (d, 1H), 8.83 (d, 1H), 7.89 (d, 1H), 7.43 (s, 2H), 7.08 (m, 2H), 4.80 (d, 2H), 4.61 (d, 2H), 3.55 (t, 2H), 2.88 (s, 1H), 1.85 (m, 4H). ³¹P NMR (300 MHz) CD₃OD ∂: 29.05. MS: 474.27 (M+H).

### Example 418

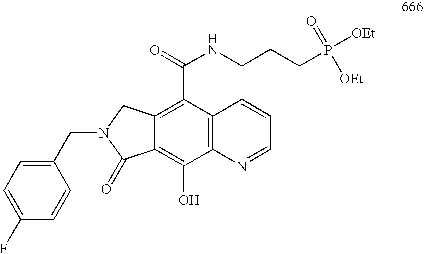

¹H NMR (300 MHz) CDCl₃ ∂: 8.86 (d, 1H), 8.58 (d, 1H), 7.60 (d, 1H), 7.24 (m, 2H), 7.01 (m, 2H), 4.47 (s, 2H), 4.42 (s, 2H), 4.15 (m, 4H), 3.69 (m, 2H), 2.01 (m, 4H), 1.29 (r, 6H). ¹⁹F NMR (300 MHz) CDCl₃ ∂: −76.35, −114.51. MS: 530.31 (M+H).

### Example 419

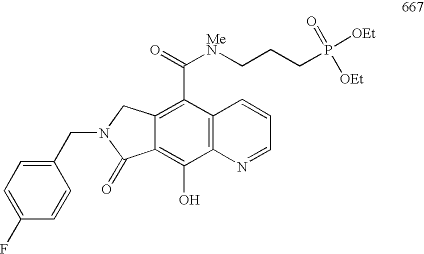

¹H NMR (300 MHz) CDCl₃ ∂: 8.98 (s, 1H), 8.10 (d, 1H), 7.62 (d, 1H), 7.23 (s, 2H), 7.09 (m, 2H), 4.88 (m, 2H), 4.70 (m, 2H), 4.23 (m, 4H), 3.92 (m, 1H), 3.73 (m, 1H) (m, 2H), 3.19 (s, N-Me rotamer, 3H), 2.78 (s, N-Me rotamer, 3H), 2.05 (m, 6H), 1.38 (m, 6H). ¹⁹F NMR (300 MHz) CDCl₃ ∂: −114.60. ³¹P NMR (300 MHz) CDCl₃ ∂: 30.95, 29.93. MS: 544.32 (M+H).

### Example 420

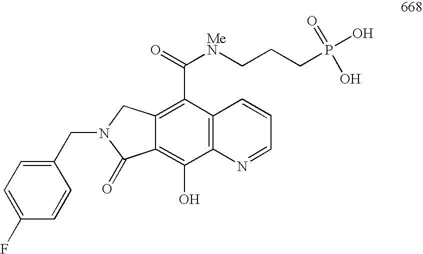

¹H NMR (300 MHz) CD₃OD ∂: 9.00 (d, 1H), 8.38 (m, 1H), 7.82 (m, 1H), 7.42 (m, 2H), 7.02 (m, 2H), 4.85 (m, 2H), 4.69 (m, 2H), 4.44 (m, 2H), 3.89 (m, 2H), 3.31 (s, N-Me rotamer, 3H), 2.66 (s, N-Me rotamer, 3H), 2.05 (m, 1H), 1.89 (m, 1H). ¹⁹F NMR (300 MHz) CD₃OD ∂: −117.28, −78.13. ³¹P NMR (300 MHz) CD₃OD ∂: 28.79, 28.19. MS: 488.20 (M+H).

### Example 421

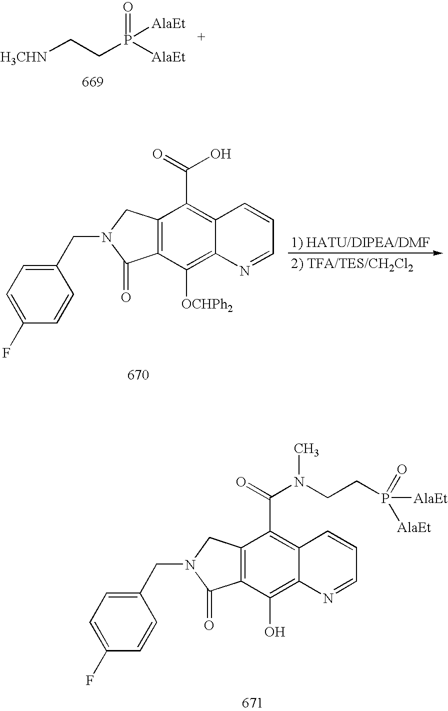

Amine 669 was prepared from procedures similar to those reported herein. Following its formation, it was then coupled to a carboxylic acid scaffold using an HATU promoted amide bond formation reaction. The diphenylmethyl ether protecting group was then removed to obtain product 671, which was isolated as a mixture of phosphorous diastereomers. ¹H NMR (300 MHz) CD₃OD Diagnostic peaks observed at δ: 8.92 (m, 1H), 8.26 (m, 1H), 2.95 (s, N-Me rotamers, 3H), 1.75 (d, 3H). ³¹P NMR (121 MHz) CD₃OD δ: 36.2, minor rotamers observed at 28.0, 26.2 ppm. MS: 672.1 (M+H).

### Example 422

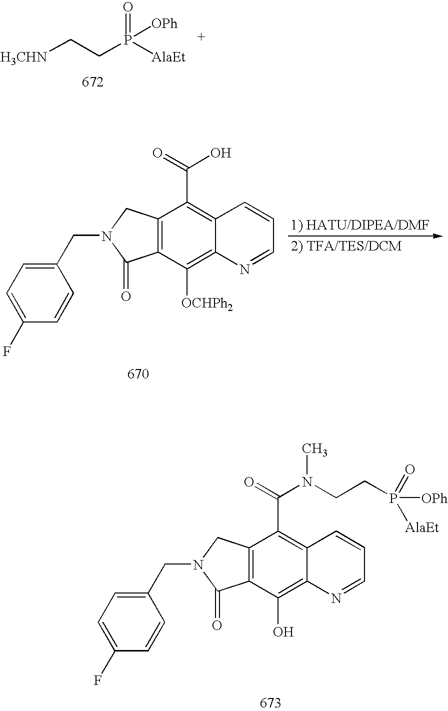

Amine 672 was prepared from procedures similar to those reported herein. Following its formation, this amine was it was then coupled to a carboxylic acid scaffold using an HATU promoted amide formation. The diphenylmethyl ether protecting group was then removed to obtain product 673, which was isolated as a mixture of phosphorous diastereomers. ¹H NMR (300 MHz) CD₃OD Diagnostic peaks at δ: 8.95 (m, 1H), 8.35 (m, 1H), 2.85-3.1 (s, N-Me rotamer, 3H), 1.42 (d, 3H). ³¹P NMR (121 MHz) CD₃OD δ 31.2, 30.0. MS: 649.4 (M+H).

### Example 423

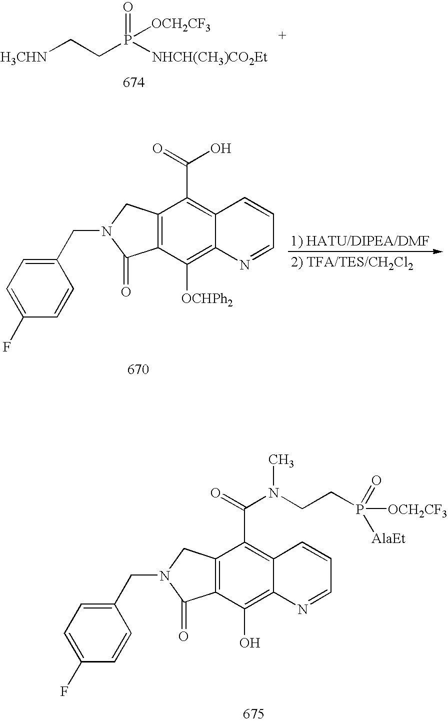

Amine 674 was prepared from procedures similar to those reported herein. Following its formation, this amine was it was then coupled to a carboxylic acid scaffold using an HATU promoted amide formation. The diphenylmethyl ether protecting group was then removed to obtain product 675, which was isolated as a mixture of phosphorous diastereomers. ¹H NMR (300 MHz) CD₃OD Diagnostic peaks at δ: 8.98 (d, 1H), 8.30-8.40 (m, 1H), 7.42 (m, 2H), 7.15 (m, 2H), 2.82 (s, N-Me rotamers). ³¹P NMR (121 MHz) CD₃OD δ 36.2, 35.0, minor rotamer observed at 34.5. MS: 655.2 (M+H).

### Example 424

### General Procedure for Synthesis of Mono-Substituted Sulfamates Using Cs₂CO₃ Promoted Alkylation

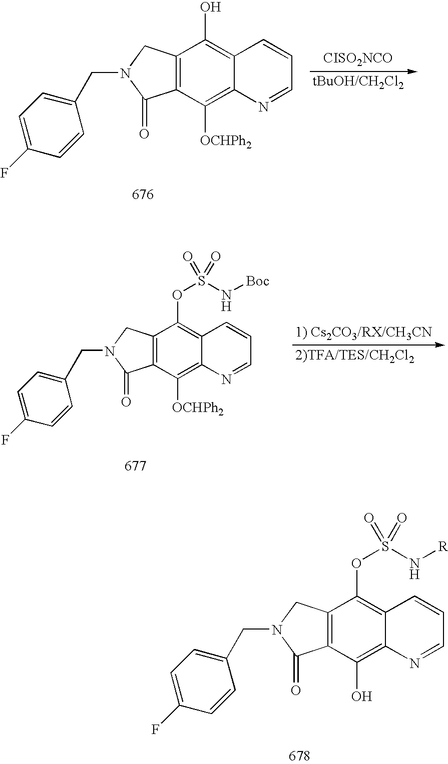

To 300 mg chlorosulfonyl isocyanate, in 10 ml CH₂Cl₂ at 0 degrees C., is added 0.3 ml of t-butanol. The reaction is then allowed to warm to room temperature. To a separate flask containing 1.2 g of phenol 676 in methylene chloride is added DIPEA (3 equiv), followed by 0.9 ml of the N-Boc sulfamyl chloride prepared above. The resulting N-Boc sulfamate 677 is used in subsequent alkylations without further purification.

To sulfamate 677 in acetonitrile is added Cs₂CO₃, followed by alkylating agent. The reaction is then heated to 80 degrees C. to effect alkylation. The resulting N-Boc N-alkyl sulfamate is subjected to TFA/TES treatment to remove both the diphenylmethyl ether and the t-butyl carbamate to furnish the final N-alkyl sulfamate products.

### Example 425

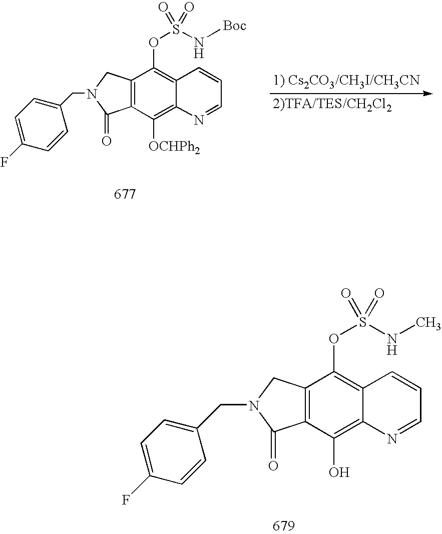

Sulfamate 674 was formed by the use of methyl iodide as alkylating agent in the general procedure described above to give N-methyl sulfamate product.

¹H NMR (300 MHz) CDCl₃ δ: 8.94 (m, 1H), 8.55 (m, 1H), 7.66 (m, 1H), 7.25-7.42 (m, 2H), 7.02-7.16 (m, 2H), 5.54 (bs, 1H), 4.85 (s, 2H), 4.62 (s, 2H), 3.08 (d, 3H). MS: 418.0 (M+H).

### Example 426

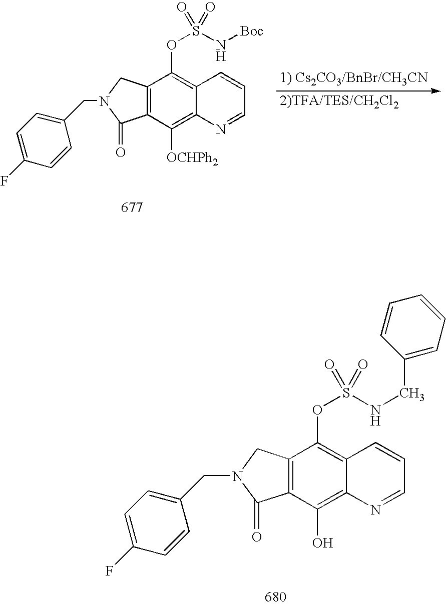

Sulfamate 680 was formed by the use of benzyl bromide as alkylating agent in the general procedure described above. ¹H NMR (300 MHz) CDCl₃ δ: 9.15 (m, 1H), 8.44 (m, 1H), 7.65 (m, 1H), 7.15-7.20 (m, 7H), 7.04 (m, 2H), 4.78 (s, 2H), 4.54 (s, 2H), 4.42 (d, 2H). MS 494.1 (M+H).

### Example 427

### General Procedure for the Synthesis of Mono-Substituted Sulfamates Using Mitsunobu Reaction

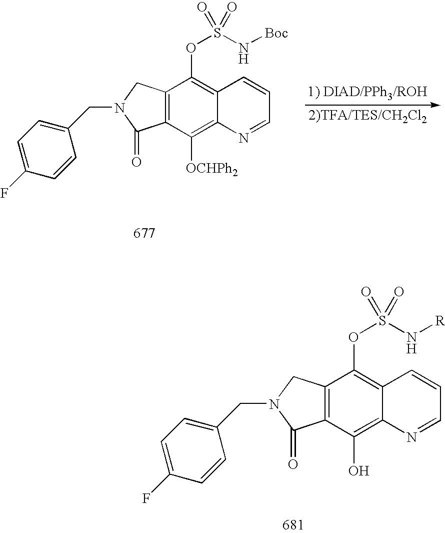

To sulfamate 677 in THF at 0 degrees C. is added PPh₃, the desired alcohol, and finally DIAD. The reaction is then allowed to warm to room temperature and the resulting product isolated by direct introduction of the reaction mixture onto a silica gel column after filtration to remove triphenylphosphine oxide. The resulting N-Boc N-alkyl sulfamate is subjected to TFA/TES treatment to remove both the diphenylmethyl ether and the t-butyl carbamate to furnish the final N-alkyl sulfamate products.

### Example 428

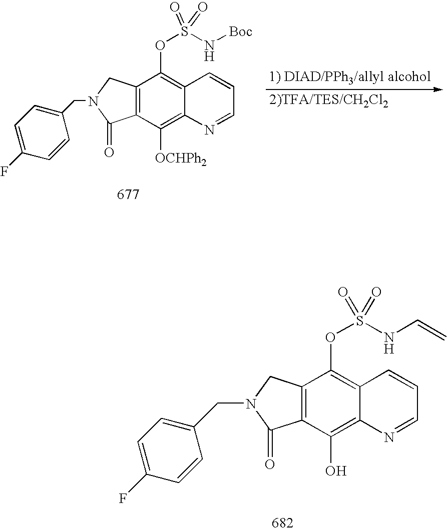

Sulfamate 682 was formed by the use of allyl alcohol as alkylating agent in the general procedure described above. ¹H NMR (300 MHz) CDCl₃ δ: 8.96 (m, 1H), 8.56 (m, 1H), 7.66 (m, 1H), 7.38 (m, 2H), 7.04 (m, 2H), 5.82-6.03 (m, 1H), 5.23-5.42 (m, 2H), 4.78 (s, 2H), 4.64 (s, 2H), 4.02 (bm, 2H). MS 444.1 (M+H).

### Example 429

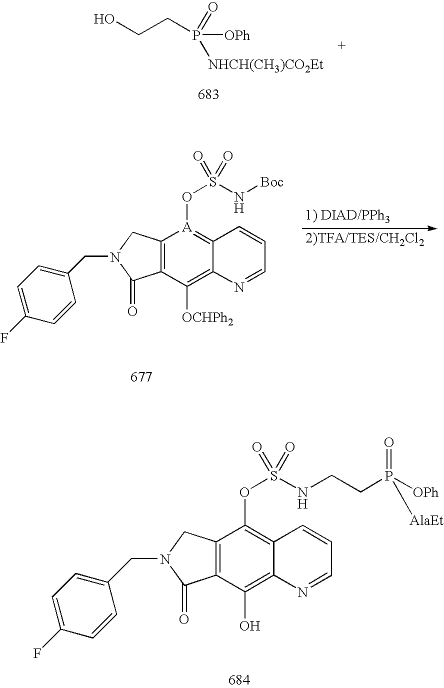

Phosphonate alcohol 683 was prepared by sodium cyanoborohydride reduction of a phosphonate aldehyde. Sulfamate 684 was formed by the use of phosphonate alcohol 683 as alkylating agent in the general Mitsunobu procedure described above to give N-methyl sulfamate product as a mixture of phosphorous diasteromers.

¹H NMR (300 MHz) CDCl₃: Diagnostic peaks at δ 9.12 (m, 1H), 8.62 (m, 1H), 7.6 (m, 1H), 4.8 (s, 2H), 4.62 (d, 2H), 4.10-4.26 (m, 2H), 3.85-3.92 (m, 2H), 1.72 (d, 3H). MS: 686.1 (M+H).

### Example 430

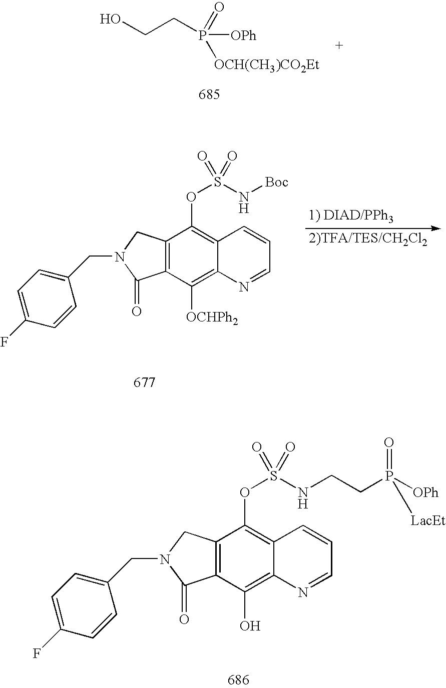

Phosphonate alcohol 685 was was prepared by sodium cyanoborohydride reduction of the corresponding phosphonate aldehyde. Sulfamate 686 was formed by the use of phosphonate alcohol 685 as alkylating agent in the general Mitsunobu procedure described above. ¹H NMR (300 MHz) CD₃OD:Diagnostic peaks observed at δ 9.25 (m, 1H), 8.64 (m, 1H), 3.62-3.75 (m, 2H), 1.82 (d, 3H), 1.20-1.43 (t, 3H). MS: 688.0 (M+H).

### Example 431

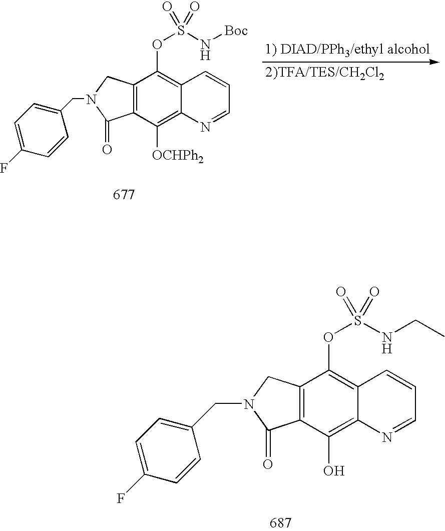

Sulfamate 687 was formed by the use of ethyl alcohol as alkylating agent in the general procedure described above. ¹H NMR (300 MHz) CDCl₃:δ 9.04 (m, 1H), 8.76 (m, 1H), 7.74 (m, 1H), 7.36 (m, 2H), 7.06 (m, 2H), 4.76 (s, 2H), 4.71 (s, 2H), 3.54 (q, 2H), 1.42 (t, 3H). MS 432.1 (M+H)

### Example 432

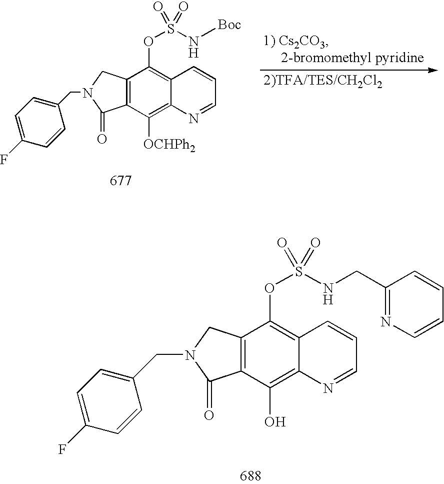

Sulfamate 688 was formed by the use of 2-bromomethylpyridine as alkylating agent in the general cesium carbonate mediated alkylation procedure described above. ¹H NMR (300 MHz) CDCl₃:δ 8.94 (m, 1H), 8.55 (m, 1H), 7.86 (m, 2H), 7.42 (m, 4H), 7.04 (m, 3H), 4.82 (s, 2H), 4.74 (s, 2H). MS 495.1 (M+H)

### Example 433

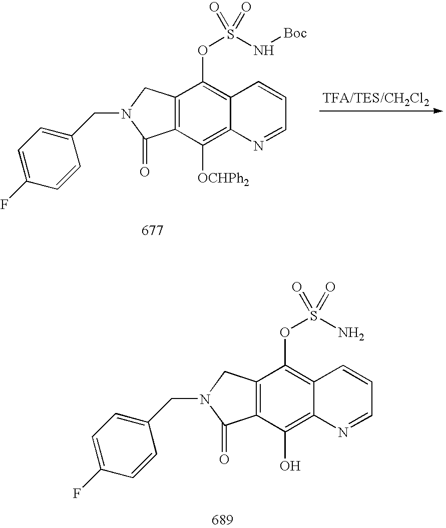

Sulfamate 689 was formed by the use of 2-bromomethylpyridine as alkylating agent in the general cesium carbonate mediated alkylation procedure described above. ¹H NMR (300 MHz) CD₃OD₃:δ 8.94 (m, 1H), 8.72 (m, 1H), 7.42 (m, 2H), 7.04 (m, 2H), 4.82 (s, 2H), 4.64 (s, 2H). MS 404.0 (M+H).

The foregoing specification teaches the principles of the present invention, with Examples provided for the purpose of illustration, and fully discloses how to make and use the present invention. The invention is not limited to the particular embodiments described herein but includes all modifications within the scope of the appended claims and their equivalents. Those skilled in the art will recognize through routine experimentation that various changes and modifications can be made without departing from the scope of this invention.

All publications, including, but not limited to, patents and patent applications cited in this specification, are herein incorporated by reference as if each individual publication were specifically and fully set forth.

We claim:

1. An HIV integrase inhibitor compound comprising a phosphonate group.

2. An HIV integrase inhibitor compound of claim 1 comprising one or more covalently attached A⁰ groups;
wherein:
A⁰ is A¹, A² or W³; 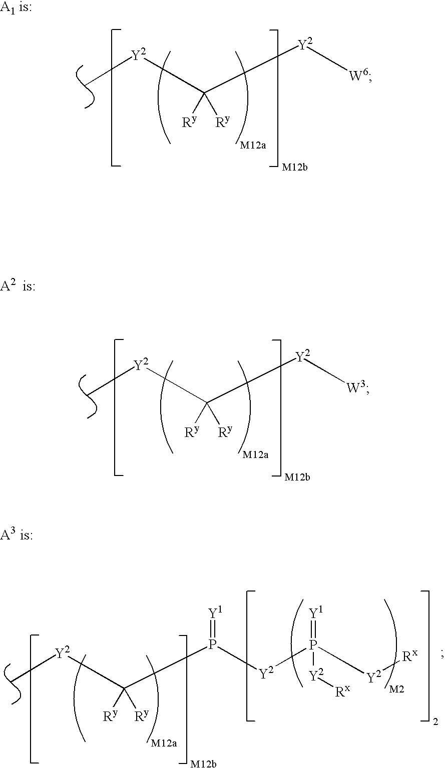
where:
Y¹ is independently O, S, NR^{x}, N(O)(R^{x}), N(OR^{x}), N(O)(OR^{x}), or N(N(R^{x})₂);
Y² is independently a bond, O, NR^{x}, N(O)(R^{x}), N(OR^{x}), N(O)(OR^{x}), N(N(R^{x})₂), S(O) (sulfoxide), S(O)₂ (sulfone), S (sulfide), or S—S (disulfide);
M2 is 0, 1 or 2;
M12a is 1, 2, 3, 4, 5, 6, 7, 8, 9, 10, 11, or 12; and
M12b is 0, 1, 2, 3, 4, 5, 6, 7, 8, 9, 10, 11, or 12;
R^{y} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or where taken together at a carbon atom, two vicinal R^{y} groups form a carbocycle or a heterocycle; or taken together at a carbon atom, two vicinal R^{y} groups form a ring; such as, cyclopropyl, cyclobutyl, cyclopentyl, or cyclohexyl; or the ring may contain one or more heteroatoms forming a heterocyclic ring such as, piperazinyl, piperidinyl, pyranyl, or tetrahydrofuryl;
R^{x} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or the formula: 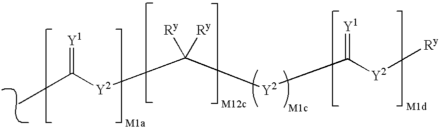
where M1a, M1c, and M1d are independently 0 or 1, and M12c is 0, 1, 2, 3, 4, 5, 6, 7, 8, 9, 10, 11 or 12;
W³ is W⁴ or W⁵;
W⁴ is R⁵, —C(Y¹)R⁵, —C(Y¹)W⁵, —SO₂R⁵, or —SO₂W⁵;
W⁵ is a carbocycle or a heterocycle wherein W⁵ is independently substituted with 0 to 3 R² groups;
W^{3a} is W^{4a} or W^{5a};
W^{4a} is R^{5a}, —C(Y¹)R^{5a}, C(Y¹)W^{5a}, —SO₂R^{2a}, or SO₂W^{5a};
W^{5a} is a multivalent substituted carbocycle or heterocycle wherein W^{5a} is independently substituted with 0 to 3 R¹ groups;
W⁶ is W^{3a} independently substituted with 1, 2, or 3 A³ groups;
R¹ is independently H or alkyl of 1 to 18 carbon atoms;
R² is independently H, R³ or R⁴ wherein each R⁴ is independently substituted with 0 to 3 R³ groups; or taken together at a carbon atom, two R² groups form a ring; such as, cyclopropyl, cyclobutyl, cyclopentyl, or cyclohexyl; optionally, the ring may be substituted with 0 to 3 R³ groups;
R³ is R^{3a}, R^{3b}, R^{3c} or R^{3d}, provided that when R³ is bound to a heteroatom, then R³ is R^{3c} or R^{3d};
R^{3a} is F, Cl, Br, I, —CN, N₃ or —NO₂;
R^{3b} is Y¹;
R^{3c} is —R^{x}, —N(R^{x})₂, —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)N(R^{x})₂, —SC(Y¹)R^{x}, —SC(Y¹)OR^{x}, —SC(Y¹)N(R^{x})₂, —N(R^{x})C(Y¹)R^{x}, —N(R^{x})C(Y¹)OR^{x}, or —N(R^{x})C(Y¹)N(R^{x})₂;
R^{3d} is —C(Y¹)R^{x}, —C(Y¹)OR^{x} or —C(Y¹)N(R^{x})₂;
R⁴ is an alkyl of 1 to 18 carbon atoms, alkenyl of 2 to 18 carbon atoms, or alkynyl of 2 to 18 carbon atoms;
R⁵ is R⁴ wherein each R⁴ is substituted with 0 to 3 R³ groups; and
R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;
R is independently selected from H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, C₂-C₂₀ substituted heterocycle, phosphonate, phosphate, polyethyleneoxy, a protecting group, L-A³, and a prodrug moiety;
Substituted alkyl, substituted alkenyl, substituted alkynyl, substituted aryl, and substituted heterocycle are independently substituted with one or more substituents selected from F, Cl, Br, I, OH, amino (—NH₂), ammonium (—NH₃⁺), alkylamino (—NHR), dialkylamino (—NR₂), trialkylammonium (—NR₃⁺), C₁-C₈ alkyl, C₁-C₈ alkylhalide, carboxylate, thiol (—SH), sulfate (—OSO₃R), sulfamate, sulfonate (—SO₃R), 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—COOR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, and a prodrug moiety; and
L is a bond, O, S, S—S (disulfide), S(═O) (sulfoxide), S(═O)₂ (sulfone), —S(═O)₂N(R)— (sulfonamide), NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, —(CR₂)ₙO(CR₂)ₙ—, —C(═O)NH—, —OC(═O)NH—, —NHC(═O)NH—, C(═O), —C(═O)NH(CH₂)ₙ—, or —(CH₂CH₂O)ₙ—, where n may be 1, 2, 3, 4, 5, or 6;
wherein at least one A⁰ group is an A¹ group.

3. An HIV integrase inhibitor compound of claim 1 comprising one or more covalently attached A¹ groups;
wherein: 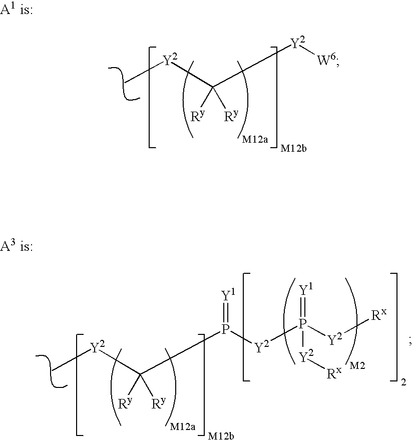
where:
Y¹ is independently O, S, NR^{x}, N(O)(R^{x}), N(OR^{x}), N(O)(OR^{x}), or N(N(R^{x})₂);
Y² is independently a bond, O, NR^{x}, N(O)(R^{x}), N(OR^{x}), N(O)(OR^{x}), N(N(R^{x})₂), S(O) (sulfoxide), S(O)₂ (sulfone), S (sulfide), or S—S (disulfide);
M2 is 0, 1 or 2;
M12a is 1, 2, 3, 4, 5, 6, 7, 8, 9, 10, 11, or 12; and
M12b is 0, 1, 2, 3, 4, 5, 6, 7, 8, 9, 10, 11, or 12;
R^{y} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or where taken together at a carbon atom, two vicinal R^{y} groups form a carbocycle or a heterocycle; or taken together at a carbon atom, two vicinal R^{y} groups form a ring; such as, cyclopropyl, cyclobutyl, cyclopentyl, or cyclohexyl; or the ring may contain one or more heteroatoms forming a heterocyclic ring such as, piperazinyl, piperidinyl, pyranyl, or tetrahydrofuryl;
R^{x} is independently H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, or a protecting group, or the formula: 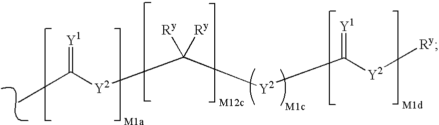
where M1a, M1c, and M1d are independently 0 or 1, and M12c is 0, 1, 2, 3, 4, 5, 6, 7, 8, 9, 10, 11 or 12;
W^{3a} is W^{4a} or W^{5a};
W^{4a} is R^{5a}, —C(Y¹)R^{5a}, —C(Y¹)W^{5a}, —SO₂R^{5a}, or —SO₂W^{5a};
W^{5a} is a multivalent substituted carbocycle or heterocycle wherein W^{5a} is independently substituted with 0 to 3 R² groups;
W⁶ is W^{3a} independently substituted with 1, 2, or 3 A³ groups;
R¹ is independently H or alkyl of 1 to 18 carbon atoms;
R² is independently H, R³ or R⁴ wherein each R⁴ is independently substituted with 0 to 3 R³ groups; or taken together at a carbon atom, two R² groups form a ring; such as, cyclopropyl, cyclobutyl, cyclopentyl, or cyclohexyl; optionally, the ring may be substituted with 0 to 3 R³ groups;
R³ is R^{3a}, R^{3b}, R^{3c} or R^{3d}, provided that when R³ is bound to a heteroatom, then R³ is R^{3c} or R^{3d};
R^{3a} is F, Cl, Br, I, —CN, N₃ or —NO₂;
R^{3b} is Y¹;
R^{3c} is —R^{x}, —N(R^{x})₂, —SR^{x}, —S(O)R^{x}, —S(O)₂R^{x}, —S(O)(OR^{x}), —S(O)₂(OR^{x}), —OC(Y¹)R^{x}, —OC(Y¹)OR^{x}, —OC(Y¹)N(R^{x})₂, —SC(Y¹)R^{x}, —SC(Y¹)OR^{x}, —SC(Y¹)N(R^{x})₂, —N(R^{x})C(Y¹)R^{x}, —N(R^{x})C(Y¹)OR^{x}, or —N(R^{x})C(Y¹)N(R^{x})₂;
R^{3d} is —C(Y¹)R^{x}, —C(Y¹)OR^{x} or —C(Y¹)N(R^{x})₂;
R⁴ is an alkyl of 1 to 18 carbon atoms, alkenyl of 2 to 18 carbon atoms, or alkynyl of 2 to 18 carbon atoms;
R⁵ is R⁴ wherein each R⁴ is substituted with 0 to 3 R³ groups; and
R^{5a} is independently alkylene of 1 to 18 carbon atoms, alkenylene of 2 to 18 carbon atoms, or alkynylene of 2-18 carbon atoms any one of which alkylene, alkenylene or alkynylene is substituted with 0-3 R³ groups;
R is independently selected from the group consisting of H, C₁-C₁₈ alkyl, C₁-C₁₈ substituted alkyl, C₂-C₁₈ alkenyl, C₂-C₁₈ substituted alkenyl, C₂-C₁₈ alkynyl, C₂-C₁₈ substituted alkynyl, C₆-C₂₀ aryl, C₆-C₂₀ substituted aryl, C₂-C₂₀ heterocycle, C₂-C₂₀ substituted heterocycle, phosphonate, phosphate, polyethyleneoxy, a protecting group, L-A³, and a prodrug moiety;
substituted alkyl, substituted alkenyl, substituted alkynyl, substituted aryl, and substituted heterocycle are independently substituted with one or more substituents selected from F, Cl, Br, I, OH, amino (—NH₂), ammonium (—NH₃⁺), alkylamino (—NHR), dialkylamino (—NR₂), trialkylammonium (—NR₃⁺), C₁-C₈ alkyl, C₁-C₈ alkylhalide, carboxylate, thiol (—SH), sulfate (—OSO₃R), sulfamate, sulfonate (—SO₃R), 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, alkylsulfone (—SO₂R), arylsulfone (—SO₂Ar), arylsulfoxide (—SOAr), arylthio (—SAr), sulfonamide (—SO₂NR₂), alkylsulfoxide (—SOR), ester (—COOR), amido (—C(═O)NR₂), 5-7 membered ring lactam, 5-7 membered ring lactone, nitrile (—CN), azido (—N₃), nitro (—NO₂), C₁-C₈ alkoxy (—OR), C₁-C₈ alkyl, C₁-C₈ substituted alkyl, C₆-C₂₀ aryl, C₆-C₂₀ substituted aryl, C₂-C₂₀ heterocycle, and C₂-C₂₀ substituted heterocycle, phosphonate, phosphate, polyethyleneoxy, and a prodrug moiety; and
L is a bond, O, S, S—S (disulfide), S(═O) (sulfoxide), S(═O)₂ (sulfone), —S(═O)₂N(R)— (sulfonamide), NR, N—OR, C₁-C₁₂ alkylene, C₁-C₁₂ substituted alkylene, C₂-C₁₂ alkenylene, C₂-C₁₂ substituted alkenylene, C₂-C₁₂ alkynylene, C₂-C₁₂ substituted alkynylene, —(CR2)ₙO(CR₂)ₙ—, —C(═O)NH—, —OC(═O)NH—, —NHC(═O)NH—, C(═O), —C(═O)NH(CH₂)ₙ—, or —(CH₂CH₂O)ₙ—, where n may be 1, 2, 3, 4, 5, or 6.

4. An HIV integrase inhibitor compound of claim 2 wherein a carbocycle and a heterocycle is independently selected from:

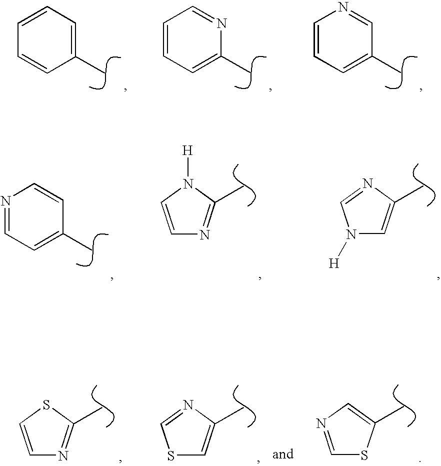

5. An HIV integrase inhibitor compound of claim 2 wherein a carbocycle is selected from:

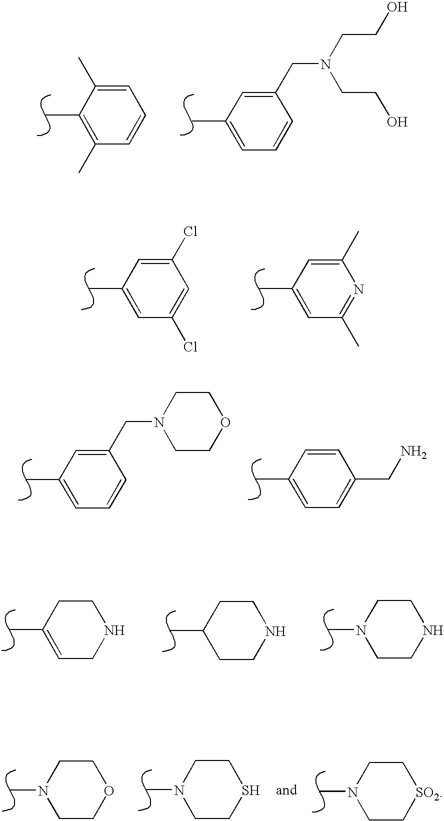

6. An HIV integrase inhibitor compound of claim 2 wherein a substituted aryl is selected from:

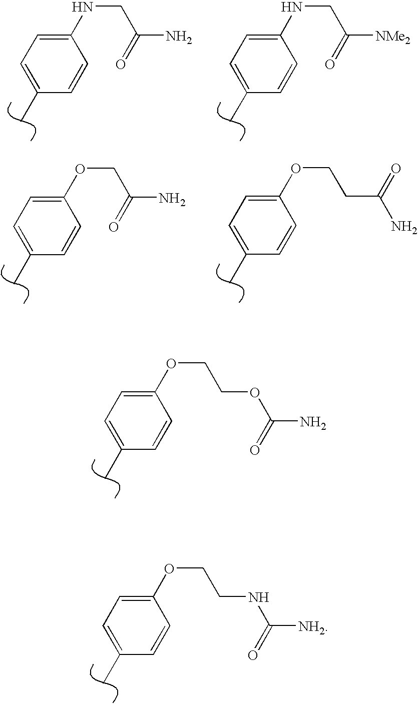

7. An HIV integrase inhibitor compound of claim 2 wherein a substituted aryl is selected from: 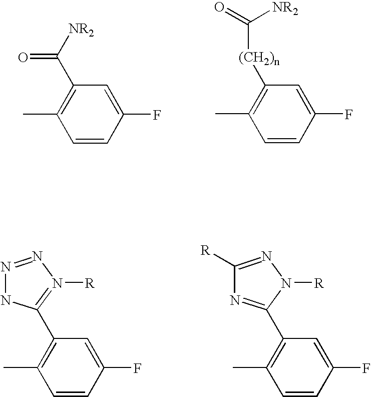
where n is 1 to 6.

8. An HIV integrase inhibitor compound of claim 2 wherein A¹ is of the formula:

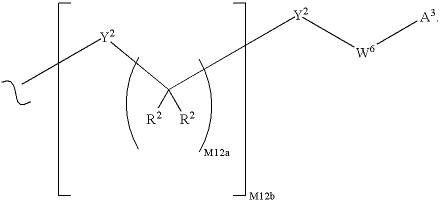

9. An HIV integrase inhibitor compound of claim 8 wherein A¹ is of the formula:

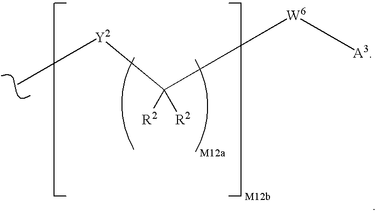

10. An HIV integrase inhibitor compound of claim 9 wherein A¹ is of the formula:

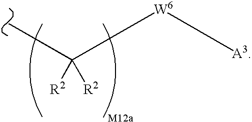

11. An HIV integrase inhibitor compound of claim 10 wherein A¹ is of the formula: 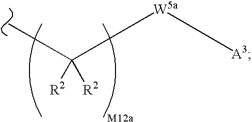
where W^{5a} is a carbocycle or a heterocycle and W^{5a} is independently substituted with 0 or 1 R² groups.

12. An HIV integrase inhibitor compound of claim 2 wherein A¹ is of the formula: 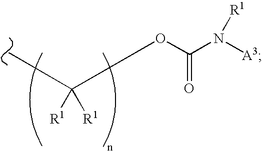
where n is an integer from 1 to 18.

13. An HIV integrase inhibitor compound of claim 2 wherein A² is of the formula:

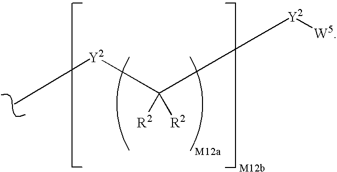

14. An HIV integrase inhibitor compound of claim 13 wherein A² is phenyl, substituted phenyl, benzyl, substituted benzyl, pyridyl or substituted pyridyl.

15. An HIV integrase inhibitor compound of claim 2 wherein A³ is of the formula:

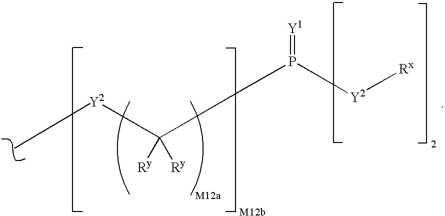

16. An HIV integrase inhibitor compound of claim 15 wherein A³ is of the formula: 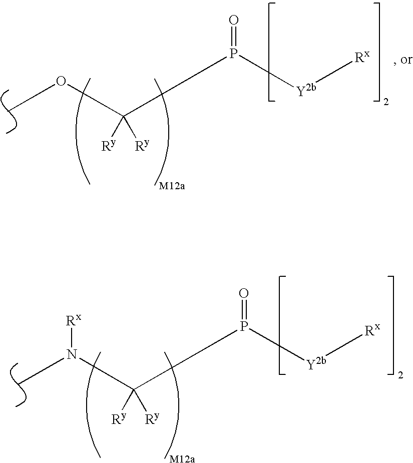
where Y^{2b} is independently oxygen (O) or nitrogen (N(R^{x})).

17. An HIV integrase inhibitor compound of claim 2 wherein A³ is of the formula: 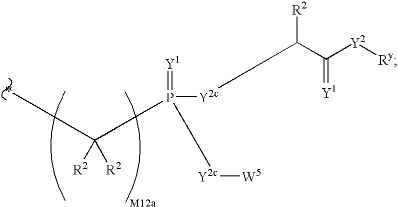
where W⁵ is phenyl or substituted phenyl, and Y^{2c} is independently O, N(R^{y}) or S.

18. An HIV integrase inhibitor compound of claim 17 where R² is H, M12a is 1, and Y^{2c} is independently O or N(R^{y}).

19. An HIV integrase inhibitor compound of claim 2 wherein A³ is of the formula: 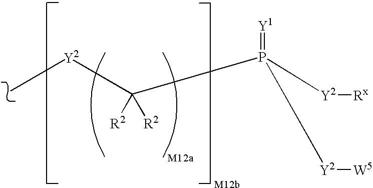
where W⁵ is phenyl or substituted phenyl.

20. An HIV integrase inhibitor compound of claim 19 where R^{x} is selected from the groups:

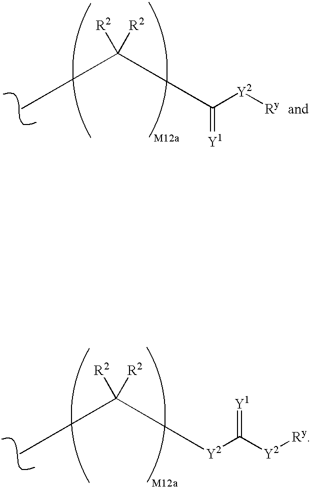

21. An HIV integrase inhibitor compound of claim 20 where A³ is of the formula: 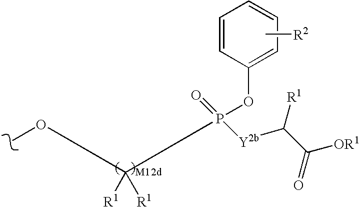
where Y^{2b} is O or N(R^{x}); M12d is 1, 2, 3, 4, 5, 6, 7 or 8; and the phenyl carbocycle is substituted with 0 to 3 R² groups.

22. An HIV integrase inhibitor compound of claim 21 where A³ is of the formula:

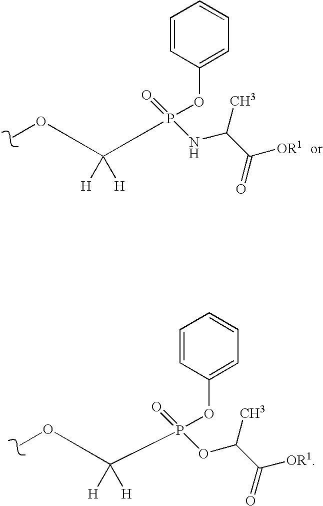

23. An HIV integrase inhibitor compound of claim 22 where A³ is of the formula:

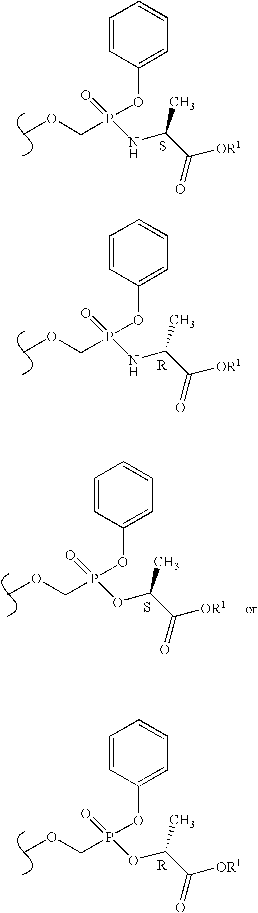

24. An HIV integrase inhibitor compound of claim 2 or 3 selected from Formula I: 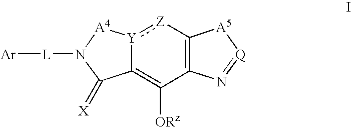
wherein:
A⁴ and A⁵ are each and independently a moiety forming a five, six, or seven membered ring; or A⁴ and A⁵ are independently selected from the group consisting of O, S, NR, C(R²)₂, CR²OR, CR²C(═O)R, C(═O), C(═S), CR²SR, C(═NR), C(R²)₂—C(R³)₂, C(R²)═C(R³), C(R²)₂—O, NR—C(R³)₂, N═C(R³), N═N, SO₂—NR, C(═O)C(R³)₂, C(═O)NR, C(R²)₂—C(R³)₂—C(R³)₂, C(R²)═C(R³)—C(R³)₂, C(R²)C(═O)NR, C(R²)C(═S)NR, C(R²)═N—C(R³)₂, C(R²)═N—NR, and N═C(R³)—NR;
Q is N, ⁺NR, or CR⁴; 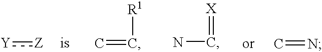
X may be O, S, NH, NR, N—OR, N—NR₂, N—CR₂OR or N—CR₂NR₂;
R^{z} is H; a protecting group selected from benzyhydryl (CHPh₂), trialkylsilyl (R₃Si), 2-trimethylsiloxyethyl, alkoxymethyl (CH₂OR), and ester (C(═O)R); or a prodrug moiety;
R¹, R², R³ and R⁴ are each independently selected from the group consisting of H, F, Cl, Br, I, OH, —NH₂, —NH₃⁺, —NHR, —NR₂, —NR₃⁺, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, —SO₂R, —SO₂Ar, —SOAr, —SR, —SAr, —SO₂NR₂ (sulfonamide), —SOR, —CO₂R, —C(═O)NR₂, 5-7 membered ring lactam, 5-7 membered ring lactone, —CN, —N₃, —NO₂, C₁-C₈ alkoxy, C₁-C₈ trifluoroalkyl, C₁-C₈ alkyl, C₁-C₈ substituted alkyl, C₃-C₁₂ carbocycle, C₃-C₁₂ substituted carbocycle, C₆-C₂₀ aryl, C₆-C₂₀ substituted aryl, C₂-C₂₀ heteroaryl, C₂-C₂₀ substituted heteroaryl, polyethyleneoxy, phosphonate, phosphate, a prodrug moiety, —OC(═O)OR, —OC(═O)NR₂, —OC(═S)NR₂, —OC(═O)NRNR₂, —OC(═O)R, —C(═O)NRNR₂, —C(═O)R, —OSO₂NR₂ (sulfamate), —NRSO₂R, —NRC(═S)NR₂, —OSO₂R (sulfonate), —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof; and
wherein when taken together on a single carbon, two R² or two R³ may form a spiro ring; and
Ar is C₃-C₁₂ carbocycle, C₃-C₁₂ substituted carbocycle, C₆-C₂₀ aryl, C₆-C₂₀ substituted aryl, C₂-C₂₀ heteroaryl, or C₂-C₂₀ substituted heteroaryl;
which is substituted with one or more covalently attached A⁰ groups.

25-38. (canceled)

39. An HIV integrase inhibitor compound of claim 2 or 3 selected from Formula II: 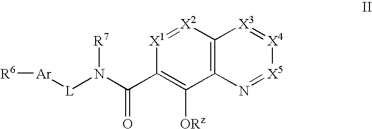
wherein:
X¹ is CR¹, NR, or N;
X² is CR², NR, or N;
X³ is CR³, NR, or N;
X⁴ is CR⁴, NR, or N;
X⁵ is CR⁵, NR, or N;
at least one of X¹, X², X³, X⁴, and X⁵ is NR or N;
R¹, R², R³, R⁴, R⁵, R⁶, and R⁷ are independently selected from the group consisting of H, F, Cl, Br, I, OH, —NH₂, —NH₃⁺, —NHR, —NR₂, —NR₃⁺, C₁-C₈ alkylhalide, carboxylate, sulfate, sulfamate, sulfonate, 5-7 membered ring sultam, C₁-C₈ alkylsulfonate, C₁-C₈ alkylamino, 4-dialkylaminopyridinium, C₁-C₈ alkylhydroxyl, C₁-C₈ alkylthiol, —SO₂R, —SO₂Ar, —SOAr, —SR, —SAr, —SO₂NR₂ (sulfonamide), —SOR, —CO₂R, —C(═O)NR₂, 5-7 membered ring lactam, 5-7 membered ring lactone, —CN, —N₃, —NO₂, C₁-C₈ alkoxy, C₁-C₈ trifluoroalkyl, C₁-C₈ alkyl, C₁-C₈ substituted alkyl, C₃-C₁₂ carbocycle, C₃-C₁₂ substituted carbocycle, C₆-C₂₀ aryl, C₆-C₂₀ substituted aryl, C₂-C₂₀ heteroaryl, C₂-C₂₀ substituted heteroaryl, polyethyleneoxy, phosphonate, phosphate, a prodrug moiety, —OC(═O)OR, —OC(═O)NR₂, —OC(═S)NR₂, —OC(═O)NRNR₂, —OC(═O)R, —C(═O)NRNR₂, —C(═O)R, —OSO₂NR₂ (sulfamate), —NRSO₂R, —NRC(═S)NR₂, —OSO₂R (sulfonate), —P(═O)(OR)₂, —P(═O)(OR)(NR₂), —P(═O)(NR₂)₂, —P(═S)(OR)₂, —P(═S)(OR)(NR₂), —P(═S)(NR₂)₂, and including prodrug substituted forms thereof;
at least one of R, R¹, R², R³, R⁴, R⁵, R⁶, and R⁷ comprises a phosphonate group; wherein the phosphonate group may be a prodrug moiety; or the phosphonate group is directly attached to a ring carbon (CR¹, CR², CR³, CR⁴ or CR⁵) of Formula II;
R^{z} is H, a protecting group, or a prodrug moiety;
Ar is C₃-C₁₂ carbocycle, C₃-C₁₂ substituted carbocycle, C₆-C₂₀ aryl, C₆-C₂₀ substituted aryl, C₂-C₂₀ heteroaryl, or C₂-C₂₀ substituted heteroaryl; and
Ar is covalently attached to L and to one or more R⁶; and
which is substituted with one or more covalently attached A⁰ groups.

40-156. (canceled)

157. A pharmaceutical composition comprising a therapeutically effective amount of an HIV integrase inhibitor compound of claim 1 and a pharmaceutically acceptable carrier.

158-162. (canceled)

163. A method of inhibiting HIV integrase, comprising the administration to a mammal in need of such treatment of a therapeutically effective amount of an HIV integrase inhibitor compound of claim 1.

164-172. (canceled)

173. A method for the treatment or prevention of the symptoms or effects of HIV infection in an animal which comprises administering to said animal a formulation comprising a therapeutically effective amount of a compound according to claim 1.

174-185. (canceled)
